(19) United States
(12) Patent Application Publication
(10) Pub. No.: US 2014/0079836 A1
(43) Pub. Date: Mar. 20, 2014

(54) METHODS AND COMPOSITIONS FOR ALTERING HEALTH, WELLBEING, AND LIFESPAN

(71) Applicant: LifeSpan Extension, LLC, Virginia Beach, VA (US)

(72) Inventor: David H. McDaniel, Virginia Beach, VA (US)

(73) Assignee: LifeSpan Extension, LLC

(21) Appl. No.: 14/084,553

(22) Filed: Nov. 19, 2013

Related U.S. Application Data
(63) Continuation of application No. 13/898,307, filed on May 20, 2013.
Application No. 13/898,307 is a division of application No. 12/629,040, filed on Dec. 1, 2009, now abandoned.
(60) Provisional application No. 61/118,945, filed on Dec. 1, 2008.

(51) Int. Cl. A61K 36/74; A61K 31/122

(52) U.S. Cl. CPC A61K 36/74; A61K 31/122
USPC 424/777; 514/690; 435/375; 506/16; 435/6.12

(57) ABSTRACT

Described herein are the results of comprehensive genetic expression and other molecular analysis of the effect of antioxidants on biological systems, including specifically different human cells. Based on these analyses, methods and compositions are described for modifying or influencing the lifespan of cells, tissues, organs, and organisms. In various embodiments, there are provided methods for modulating the activity of the gene maintenance process in order to influence the length and/or structural integrity of the telomere in living cells, as well as methods for modulating the rate/efficiency of the cellular respiration provided by the mitochondria, mitochondrial biogenesis, and maintenance of the mitochondrial membrane potential. Exemplary lifespan altering compounds include natural and synthetic antioxidants, such as plant antioxidant and polyphenol compounds derived from coffee cherry, tea, berry, and so forth, including but not limited to caffeic acid, chlorogenic acid, ferulic acid, quinic acid, proanthocyanidins, ubiquinone, idebenone, or a synthetic form or derivatives thereof.

### CROSS REFERENCE TO RELATED APPLICATION

This application is a continuation of co-pending U.S. application Ser. No. 13/898,307, filed May 20, 2013, which is a divisional of U.S. application Ser. No. 12/629,040, filed Dec. 1, 2009, and which claims the benefit of the earlier filing date of U.S. Provisional Application No. 61/118,945, filed Dec. 1, 2008. The entire content of these previously filed applications is incorporated herein by reference.

### FIELD

Described herein are methods and compositions for altering mitochondrial biogenesis and/or mitochondrial maintenance, respiratory efficiency, DNA maintenance, DNA repair, gene expression, and/or gene function, for instance in order to (in various embodiments) increase, extend, or shorten the lifespan and/or retard or increase rate of senescence of a cell, tissue, organ, and/or organism. In example embodiments, this involves altering the maintenance or function of telomeres and telomere structure, maintenance and control, cellular responses to oxidative stress and/or oxidative DNA damage, and cellular response to environmental damage or disease or immune response or genetic alteration of cells.

### BACKGROUND

All living cells and organisms have a finite lifespan. They live for a period of time and die. Cells and organisms have both a chronological age and a biological age. The former is measured in days, months or years while the latter may be measured by a host of complex testing of biological functions including but not limited to: gene expression, protein production or metabolic pathways. The rate of aging may also be measured, and an accelerated rate of aging may be considered ‘premature aging’, while a slower rate of aging may extend lifespan. It is desirable to maximize the healthy lifespan of cells and organisms and it is also desirable to extend the healthy lifespan by delaying the rate of aging and the onset of dysfunctional or disease states. Shortening the lifespan and/or accelerating apoptosis of unhealthy, diseased, damaged, or cancerous cells may also be desirable.

Oxidative stress is one of the primary causes of cell and organism dysfunction or disease and also accelerated or premature aging and death. The ability to enhance in a favorable manner the ability of cells and organisms to resist or repair damage due to oxidative stress produced by environmental injury, lifestyle choices as well as diseases and medical therapies may extend the healthy function and/or lifespan and/or retard aging and senescence. Antioxidants have the potential not only to neutralize reactive oxygen species, but also may provide vital anti-aging benefits by affecting various other key cellular mechanisms. One such example is the telomere (and/or telomere unit and associated proteins and structural configurations) which are special chromatin structures at the end of chromosomes. Telomeres are coated by DNA binding proteins, including TRF1 and TRF2 and associated proteins, TIN2, TPP1, POT1, Tankyrase 1, and Rap1 Premature or accelerated telomere shortening may produce premature aging and death. Telomerase is a DNA polymerase which plays an essential role in protecting these regions, but which may also be associated with cancer. Thus the ability to modulate telomerase activity provides the opportunity to alter health both positively and negatively.

One way to extend the lifespan of a living cell—and by extension possibly the organ, tissue or entire organism—is to repair damage in addition to preventing damage. The genes which control the cellular repair mechanisms, if activated or enhanced in the proper way, may effectively extend the lifespan of a cell. This may take several forms: extending the lifespan of a cell which is damaged or injured by properly repairing that damage and/or by causing the cell to live longer or replicate itself longer than it would have occurred naturally.

Mammalian mitochondria are organelles that produce more than 90% of cellular ATP under aerobic conditions through a process called oxidative phosphorylation. Mitochondria are also involved in fatty acid metabolism, hormone production, ketone body production, apoptosis, and Ca²⁺ homeostasis. Mitochondria contain, inter alia, the TCA cycle (also known as the Kreb cycle), enzymes involved in heme biosynthesis and the electron transport chain (OXPHOS system). Due to the large flux of redox reactions necessary to maintain oxidative phosphorylation, the organelle is the site of production of reactive oxygen species (ROS), which in controlled production have a signaling function, but in overproduction are toxic and are believed to be the cause of many human diseases including, for example, Parkinson's disease and other neurodegenerative conditions, diabetes, and the aging process itself.

The OXPHOS system is composed of five large multi-protein enzyme complexes, which collectively transform the reducing energy of NADH and FADH₂ to ATP. NADH ubiquinone oxidoreductase (Complex I) contains 45 different subunits, and succinate ubiquinone reductase (Complex II), ubiquinone-cytochrome c oxidoreductase (Complex III), cytochrome c oxidase (Complex IV) and the ATP synthase (Complex V) have 4, 11, 13 and 16 subunits respectively. Although composed of five individual enzyme complexes (each, an “OXPHOS complex” or “OXPHOS enzyme”) and containing a total of approximately 89 subunit proteins (each, an “OXPHOS protein”), the OXPHOS system has traditionally been considered to function as a single unit. This single-unit concept has been supported with evidence of structural associations between complexes, which associations are believed to enhance overall functional efficiency (Chen et al., J. Biol. Chem., 279:31761-31768, 2004; Ko et al., J. Biol. Chem., 278:12305-12309, 2003).

Four of the OXPHOS enzyme complexes (Complexes I, III, IV and V) have a dual genetic origin. That is, they are composed of both nuclear DNA-encoded proteins and mtDNA-encoded proteins. Thus, 7 subunits of Complex I, 1 subunit of Complex III, 3 subunits of Complex IV and 2 subunits of Complex V are encoded by mtDNA.

Mitochondria contain their own DNA (mtDNA) which is prokaryote-like. In mammals, this DNA is a 16 kb double-stranded circular DNA encoding 13 different polypeptides, all involved in oxidative phosphorylation, along with 2 rRNAs and 22 tRNAs. mtDNA lacks protective histones and has minimal repair mechanisms, which leads to a relatively high mutation rate that is further enhanced by the proximity of the DNA to the OXPHOS system, the site of production of ROS. Accumulation of mutations and deletions in mtDNA occurs throughout life in humans and becomes physiologically relevant where they affect sufficient number of copies of the mtDNA to alter oxidative phosphorylation.

Unlike the nuclear genome, which is present in two copies, mtDNA is present in thousands of copies in mammalian cells, all of which are used in translation of gene products made within the organelle on bacterial-like ribosomes. Thus, inheritance and penetrance of mtDNA mutations is not Mendelian, but rather depends on the relative amount (%) of wild-type and mutant mtDNA molecules per cell. The normal state is 100% wild-type mtDNA or wild-type homoplasmy. A mutation in mtDNA can also be homoplasmic (present in all mtDNA molecules of a cell) in which case it is likely to have a functional and possibly pathogenic effect. The presence of a mixture of mutant and wild-type mtDNA molecules in an individual cell is referred to as heteroplasmy. Because normal cells have an excess capacity of mtDNA and mtDNA-encoded proteins, heteroplasmic mutant mtDNA are believed to cause an altered functional (or pathogenic) phenotype if the mutant mtDNAs are present at levels exceeding some threshold value, usually 70-90%. An additional consequence of heteroplasmy is the development of altered functions of mitochondria within a single cell, between cells and between tissues (Wallace, Science, 283:1482-1488, 1999; Chinnery and Turnbull, Mol. Med. Today, 6:425-432, 2000).

Transient ischemia (anoxia) results in the local production of extremely high levels of ROS which can cause long term damage to mitochondria. Ironically, it is the sudden re-supply of oxygen to the ischemic tissue during reperfusion that is believed to be the proximate cause of elevated ROS production. In the initial phase of transient ischemia, oxygen is scarce but tissue demands for ATP remain high, resulting in continued functioning of the electron transport chain except for the terminal reduction of oxygen to water by Complex IV. Therefore, reduced electron acceptors “upstream” of Complex IV accumulate to abnormally high levels. Upon resupply of oxygen, these excess reduced carriers react directly (inappropriately) with oxygen to generate highly toxic partially reduced oxygen species (Pitkanen and Robinson, J. Clin. Invest., 98:345-351, 1996; Genova et al., FEBS Lett., 505:364-368, 2001), which are capable of protein, lipid and DNA modifying reactions. The resulting oxidative damage would be expected to occur mainly inside the mitochondrion, because such radicals are so reactive that they are short lived and cannot diffuse far before finding a target for reaction. Accordingly, OXPHOS proteins and mtDNA are likely to be the cellular molecules most affected by such oxidative stress. The resulting defects in mtDNA and OXPHOS proteins may result in continued increased production of ROS, which may also lead to a damaging positive feedback loop.

Oxidative stress is one of the primary causes of cell and organism dysfunction or disease and also accelerated or premature aging and death. Mitochondrial function or dysfunction, biogenesis, death and regenesis also play a vital role in the aging process. The ability to enhance in a favorable manner the ability of cells and organisms to resist or repair damage due to oxidative stress produced by environmental injury, lifestyle choices as well as diseases and medical therapies may extend the healthy function and/or lifespan and/or retard aging and senescence.

The ability to extend or prolong lifespan (both healthy and less healthy) lies in the ability to extend the lifespan of cells, both differentiated specialized cells and also undifferentiated stem and progenitor cells so that cell lifespan is longer or so that new cells replace senescent cells which lose their function or die. A cell normally has a finite lifespan determined by the number of cell divisions which are possible. The Hayflick Limit theory discusses one view of lifespan limitations. An organ may be repopulated with cells to regenerate itself from the stem cell population but the stem and progenitor cells themselves have a finite lifespan. The ability to extend the lifespan of differentiated cells and/or stem and progenitor cells lies at the heart of extending lifespan of an organism.

### SUMMARY

Provided herein are methods and compositions that can be employed to increase telomerase activity, and/or modulate the activity of other telomere maintenance genes so as to repair, maintain or lengthen telomere structure to lengthen the lifespan of healthy cells. Decreasing telomerase activity in cancer cells, thus making cancer cells mortal and healthy cells longer lasting if not immortal is another method to increase longevity. This disclosure describes methods of increasing or decreasing telomerase activity in healthy and stressed cells using antioxidant(s) that modulate gene activity and/or proteins which influence, regulate, and/or control telomerase activity, the maintenance of the telomere unit and associated components, or telomere length.

Exemplary compounds and compositions useful in the methods described herein include natural and synthetic antioxidants, such as plant antioxidant compounds derived from coffee cherry (e.g., including one or a mixture of caffeic acid, chlorogenic acid, ferulic acid, quinic acid and proanthocyanidins or derivatives thereof); plant antioxidant compounds derived from and plant antioxidant compounds derived from any of the plants listed herein. In another illustrative embodiment, the lifespan or health enhancing compound is synthetic/bioengineered idebenone or an ester or derivative thereof. In certain embodiments, if the modulating compound is a naturally occurring compound, it may not be in a form that is naturally occurring, for instance it may be a synthetic form or an analog or derivative of the naturally occurring form.

Importantly, embodiments of the methods and compositions described herein provide aspects of healthy longevity—that is, extended life span (of cells, tissues, organs, and/or organisms) that is healthy and of high relative quality.

Thus, in various embodiments there are provided methods for modulating: the rate/efficiency of cellular respiration provided by mitochondria, the total number of mitochondria per cell (mitochondrial biogenesis), and mitochondrial membrane potential. Also provided herein are methods for modulating the activity of the gene maintenance process, for instance for maintaining (or repairing) the length and/or structural integrity of the telomere in living cells.

Also provided herein are methods for extending the lifespan of living cells, tissues, organs or organisms. In another embodiment a method of shortening the lifespan of diseased, unhealthy or cancerous cells is described.

Presented herein are compositions and methods for administering the life-span and/or health modulating compound so that it contacts the living cells(s), thereby increasing (or in some embodiments, decreasing) the lifespan or health of the cell, the tissue in which the cell is present, and/or the organ or organism in which the cell is present.

A cell may be contacted with a modulating compound alone or in combination with other modulating compounds or synergistic non-modulating compounds which may enhance delivery to the contact cell or which may indirectly enhance the modulating effect by altering a related cellular process which then facilitates the activity of the modulating compound.

Embodiments described herein utilizes (conventional and novel) antioxidant compounds to directly modulate the gene expression of genes/proteins and complexes vital to the maintenance of telomere length.

The foregoing and other objects, features, and advantages of the invention will become more apparent from the following detailed description, which proceeds with reference to the accompanying figures.

### BRIEF DESCRIPTION OF THE DRAWINGS

FIG. 1 shows a pictorial representation of the telosome/shelterin complex and telomere structure (Multani et al., J Cell Sci 120:713-721, 2007). (A) The telomere folds back onto itself to form a double-stranded t-loop and a single-stranded D-loop. This complex protects telomeres at the G2 phase of the cell cycle from inappropriate NHEJ- and HR-mediated processing of telomeric DNA. The six-component telosome/shelterin is shown schematically on the t-loop, with POT1 interacting with the D-loop. (B) During DNA replication, the presence of WRN at the replication fork is postulated to enable the replication complex to efficiently replicate telomeric DNA. (C) The presence of WRN at telomeres may facilitate unwinding of the D-loop, enabling telomerase to extend telomeres. The linear 3′ overhang is probably protected by POT1.

FIG. 2A-2D shows biosynthetic relationships among stress-induced phenylpropanoids (derived from information in Dixon et al., The Plant Cell 7:1085-1097, 1995).

FIG. 3 shows a diagrammatic representation of a coffee cherry fruit.

FIG. 4 shows representative effects of environmental injury that lead to premature aging.

FIG. 5 shows a representation of various agents of premature aging and the multiple pathologies they can generate.

FIG. 6 shows a pictorial representation of representative mechanisms of action of premature aging in skin.

FIG. 7 is a graph showing the average expression values of three separate PCR primer assays for five longevity genes (TPP1, TERF1, TERF2, TINF2, and) in cultured human skin fibroblasts 24 hours post exposure to the listed antioxidant compounds (green tea, idebenone, or coffee cherry extract).

FIG. 8 is a graph showing the change in the number of mitochondria in human cardiac myocytes in response to COFFEEBERRY® treatment at 24 and 48 hours. As indicated, five serial dilutions of COFFEEBERRY® were used.

FIG. 9 is a graph illustrating the relative change in expression of VEGFA in human fibroblasts 24 hours after exposure to coffee cherry.

FIG. 10 is a graph illustrating the relative change in expression of HMOX1 in human fibroblasts 24 hours after exposure to coffee cherry.

For FIGS. 11-17, treatment 1=0.00001% CoffeeCherry, 0.0000005% Chlorogenic Acid; 2=0.0001% CoffeeCherry, 0.00005% Chlorogenic Acid; and 3=0.01% CoffeeCherry, 0.005% Chlorogenic Acid.

FIG. 11 is a graph illustrating the relative change in expression of CCL4L1 in human fibroblasts 24 hours after exposure to chlorogenic acid or coffee cherry.

FIG. 12 is a graph illustrating the relative change in expression of DDC in human fibroblasts 24 hours after exposure to chlorogenic acid or coffee cherry.

FIG. 13 is a graph illustrating the relative change in expression of NOS2A in human fibroblasts 24 hours after exposure to chlorogenic acid or coffee cherry.

FIG. 14 is a graph illustrating the relative change in expression of SIRT1 in human fibroblasts 24 hours after exposure to chlorogenic acid or coffee cherry.

FIG. 15 is a graph illustrating the relative change in expression of TERT in human fibroblasts 24 hours after exposure to chlorogenic acid or coffee cherry.

FIG. 16 is a graph illustrating the relative change in expression of PTGS2 in human fibroblasts 24 hours after exposure to chlorogenic acid or coffee cherry.

FIG. 17 is a graph illustrating the relative change in expression of IFI44 in human fibroblasts 24 hours after exposure to chlorogenic acid or coffee cherry.

FIG. 18 is a graph illustrating the relative change in expression of SIRT1, SIRT2, SIRT3, and SIRT4 in human fibroblasts 24 hours after exposure to different levels of coffee cherry.

FIGS. 19(a), 19(b), and 19(c) is a set of three graphs illustrating the relative change in relative expression of select genes (custom Array 2) in human skin fibroblasts 24 hours after exposure to chlorogenic acid.

FIG. 20(a) through 20(h) is a set of graphs illustrating the relative change in expression of select genes (custom Array 2) in human fibroblasts 24 hours after exposure to coffee cherry.

FIG. 21 is a pair of graphs illustrating the relative expression of genes in the mitochondrial pathway in skin fibroblasts 24 hours after exposure to (a) chlorogenic acid or (b) coffee cherry.

FIG. 22 is a pair of graphs illustrating the relative expression of select genes in the DNA repair pathway in skin fibroblasts 24 hours after exposure to (a) coffee cherry or (b) chlorogenic acid.

FIG. 23 is a graph illustrating the relative expression of select genes in the telomere maintenance pathway in skin fibroblasts 24 hours after exposure to coffee cherry.

FIG. 24 is a graph illustrating the relative expression of PARP genes in human skin fibroblasts 24 hours after exposure to coffee cherry.

FIG. 25 is a graph illustrating the relative expression of specific genes in human skin fibroblasts 24 hours after exposure to chlorogenic acid which demonstrate a classic bell shaped pattern for dose response that indicates a single directional change and then return to baseline after a peak expression level. As the doses increase, the gene response either increases or decreases until a peak expression level is reached. Beyond that dosage any increases in concentration of the compound gives “diminishing returns” or a lessening of the effect. This effect is either an upregulation or a downregulation, not bi directional.

FIG. 26 is a graph illustrating the relative expression of specific genes in human skin fibroblasts 24 hours after exposure to chlorogenic acid which demonstrate a classic bell shaped pattern for dose response that begins as a negative expression value and as the dosage increases it passes through the zero expression value and has an positive expression value until a threshold dose is reached and then returns to the other side of the axis similar to the starting dose. This is the first type of bi-directional dose response noted.

FIG. 27 is a graph illustrating the relative expression of specific genes in human skin fibroblasts 24 hours after exposure to chlorogenic acid which demonstrate a classic bell shaped pattern for dose response that begins as a positive expression value and as the dosage increases it passes through the zero expression value and has an negative expression value until a threshold dose is reached and then returns to the other side of the axis similar to the starting dose. This is the second type of bi-directional dose response noted.

### DETAILED DESCRIPTION

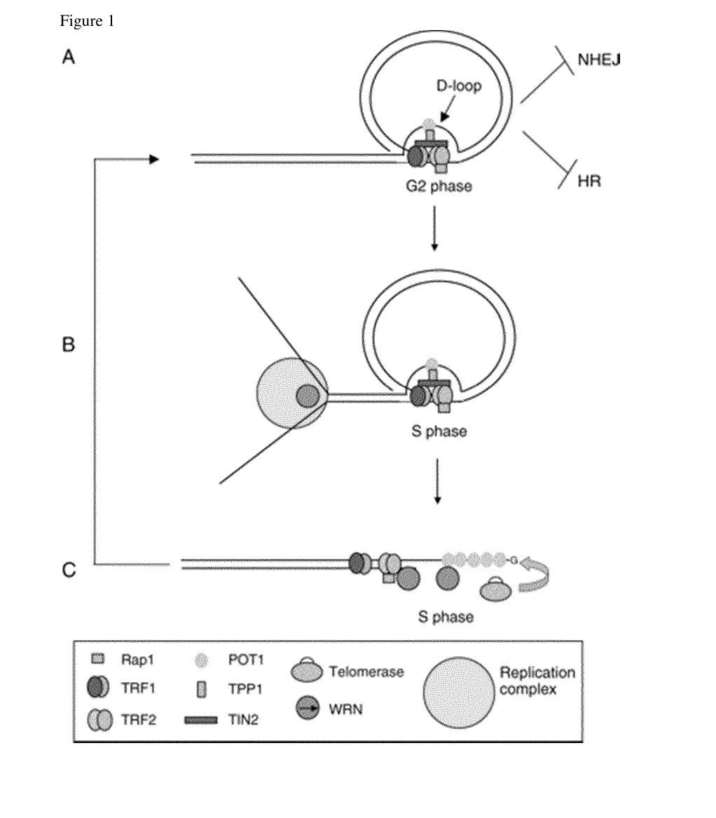

Telomeres are structures at the end of chromosomes that undergo shortening with cell division; they are consider a biological clock of sorts for how many cycles of cell replication may occur (FIG. 1). They are protective structures similar to the plastic cap on the end of shoelaces which prevent them from unraveling. With each cell division these telomere structures shorten, and this shortening accompanies aging. Eventually after the telomere shortens to a certain level the cell can no longer divide, its metabolism slows down, it ages and eventually dies.

After birth, telomerase activity is diminished; but in embryonic stem and progenitor cells, telomerase is activated and maintains telomere length and cellular immortality. However, the level of telomerase activity is low or absent in the majority of stem and progenitor cells regardless of their proliferative capacity.

Thus, even in stem and progenitor cells, except for embryonal stem and progenitor cells and cancer stem and progenitor cells, telomere shortening occurs during replicative ageing, possibly at a slower rate than that in normal somatic cells. This telomere limit prevents cell survival after extensive proliferation and thereby inhibits malignant transformation or survival, but in combination with certain other gene expression changes (such as deficient expression of the p53 tumor suppressor) then it may facilitate tumor formation or expansion.

Telomere shortening not only accompanies normal aging, but dysfunction of the telomere unit is associated with some premature aging syndromes and various diseases including aplastic anemia and many other diseases.

Telomerase is a reverse transcriptase repair enzyme which can replace lost telomere DNA structure. Typically the activity of telomerase is low, but it is a critical factor in maintaining telomere length. The activation of telomerase may rejuvenate cells and thus tissues, organs or organisms and the modulation of telomerase activity has many applications in medicine and for extending lifespan.

Alternate, telomerase independent, recombination based pathways are also a method by which cellular lifespan can be lengthened. In this method of telomere maintenance, originally discovered in telomerase defective yeast strain S. cerevisiae EST1, genetic recombination of break induced replication adds G rich telomeric repeats to the end of, or a break induced replication occurs between a critically (but still viable, i.e. retaining the repeat segments) short telomere and another portion of the telomere, essentially “lengthening” the telomere unit. The fact that these critically short telomeres are so recombinogenic, has caused speculation that the telomeres either: 1) become more recombinogenic in response to the absence of telomeres, 2) critically short telomeres trigger recombination events, or 3) critically short telomeres are preferred substrates for specific types of recombination. Recent evidence suggests that there are 2 recombination pathways and that they are characterized RAD50 and RAD51, genes that encode proteins essential for double stranded DNA break repair. Break Induced Replication (BIR) can then lengthen the telomere by the above described processes. This genomic instability, leads to breaks in the double stranded DNA which must be repaired (this repair mechanism has been shown to be inhibited by KU70). In human cells these specific genetic requirements are not known, however numerous studies demonstrated that human chromosome termini are subject to enhanced levels of recombination, as demonstrated in the yeast ALT pathway studies. A study in human cells demonstrated that the action of telomerase can effectively inhibit the alternate recombination pathway by maintaining genomic stability. Cells relying on the recombination based pathway for telomere maintenance were forced to express telomerase. These cells never demonstrated the shortened telomeres required for initiation of recombination, due to the expressed telomerase preventing such an occurrence. Other alternate pathways or mechanisms may also exist.

A recent study has also demonstrated that over expression of TERT, the catalytic subunit of telomerase protects fibroblasts against oxidative stress. When the cell is under oxidative stress, as supposed in the free radical theory of aging, the mitochondrial membrane loses potential, and mtDNA is damaged as the ion levels increase. TERT functions primarily to maintain the length of the telomere, but in cells under chronic oxidative stress, cells overexpressing TERT lose telomere length at only a slightly lesser rate than similarly stressed, non expressing cells. It has also been demonstrated in the same study that TERT is (reversibly) released from the nucleus in a dose/time dependant fashion, where it co localizes with the mitochondria. In these TERT overexpressing cells, mtDNA is protected, mitochondrial membrane potentials are higher, and concentrations of free radicals are lower which indicates better mitochondrial function/viability and decreased damage.

The activity of the enzyme telomerase is the best understood mechanism for maintaining the length of the telomere unit or structure. Modulating the activity of telomerase is one method for extending the lifespan of living cells. While normally repressed in human somatic cells it may be activated by certain repair mechanisms, certain agents and also in tumor progression or transformation.

Agents which can be utilized to modulate or alter the gene expression of this telomerase complex or its subunits can play a vital role in extending (or shortening) the lifespan of living cells. Such agents which extend the lifespan of living cells may be administered in many forms and may be used to treat disease as well as to maintain and promote health. These living cells may range from human or animal cells to plants and any other living cell. Understanding the telomerase complex is critical then to selecting agents to modulate the activity of telomerase.

One subunit of interest is Telomerase Reverse Transcriptase (TERT) which is a catalytic subunit; additional genes of interest are listed below. TERT gene expression then is controlled by Sp1 and c-myc transcription factors (genes which interestingly are frequently altered in human tumors).

The gene designated SP1 (or Transcription Factor SP1, Specificity Protein 1), when overexpressed in humans, has been shown to induce apoptosis. The apoptotic pathways involved required the binding of SP1 to the DNA (via a zinc finger domain) and were generally cell type specific. The SP1 regulated apoptosis involved alteration (downregulation) of BCLXL and BAX, no other caspases or BCL2 related genes were affected. It is involved in gene expression in the early development of an organism and when bound regulates transcription.

The gene designated as cMyc codes for a protein that binds to the DNA of other genes and modulates the activity or transcription. It is estimated that cMyc transcription factor regulates about 15% of all genes. Induction of cMyc promotes cell proliferation/transformation by binding/activating growth promoting genes. When cMyc is overexpressed or mutated the DNA binding doesn't occur correctly and cancer can result. cMyc is activated via many pathways (WNT, SHH, and EGF to name a few) and modifies the expression of many target genes resulting in a diverse number of biological effects. cMyc has also demonstrated direct activation of telomerase by inducing expression of TERT. It has been discovered that along with transcription, cMyc can affect cell growth, differentiation, stem cell self renewal and apoptosis. It is often found to be upregulated in many cancers.

The mitochondria function as the primary producer of chemical cellular energy through the production of adenosine triphosphate (ATP) via the electron transport system. By the transfer of electrons down a gradient ATP is formed for use in powering the cell. Additionally the mitochondria function in other aspects of cellular regulation, for example, the mitochondrial membrane potential (the amount of potential to move ions across the membrane to facilitate energy production) is a key regulator of apoptosis, or programmed cell death. The proton pump capacity of the membrane aids in reduction of compounds leading to energy production, and also helping regulate oxidative stress caused by free radicals. The mitochondria can also produce oxidative stress if, in the process of cellular respiration, the electrons are not transferred from Complex I to Complex III. In essence, a back log is formed and the generation of free radicals is the result. The ubiquinone, and synthetic idebenone compounds and derivatives serve to alleviate this potential backlog by serving as electron carriers/transfer agents facilitating the transition from Complex I and down the respiratory chain.

The free radical theory of aging postulates that aging and related degenerative conditions are caused by the negative effects of free radicals (highly reactive molecules or atoms that have an unpaired electron in an outer orbital that is not contributing to molecular bonding {“free”}) on cells and tissues. The free radicals are formed as byproducts of catalyzing molecular oxygen inside the cell with oxidative enzymes, and also in the connective tissues by traces of metals like iron, cobalt and manganese. The most common free radicals are the superoxide ion (O₂), the hydroxyl radical (OH), and lipid peroxyl radicals (LOO). The hydroxyl radical is highly reactive (short half life) and covalent cross linking is the most common effect. This cross linking can also perpetuate the cycle by creating more free radicals. Superoxide ions are even more reactive and found mainly in the cell cytoplasm (less often the nucleus). Hydrogen peroxide molecules are more stable and capable of passing through cellular and nuclear membranes and forming hydroxyl radicals with metals. Proteins in direct contact with hydrogen peroxide molecules can also be damaged and hydrogen peroxide is one of the most common methods for putting cells under oxidative stress. Lipid peroxidation of polyunsaturated fatty acids is a contributing factor to food becoming rancid. In living animal cells lipid membranes that have undergone peroxidation become rigid and more permeable eventually leading to cytoplasmic ion leakage and cell apoptosis.

The mitochondrial theory of aging states that the mitochondria (the organelles in the cell that create energy through the electron transport system and ATP synthase), lose function due to damage and changes to the mitochondrial DNA which codes for the proteins of the electron transport system. (This is similar to the Free radical theory of aging, but with a broader scope looking at the genetics, bioenergetics and membrane potential of the cell: not just the Free Radical chemistry). This decreased function allows for inability to process the free radicals created during the cellular respiration process and causes further oxidative damage (membrane permeability, loss of membrane potential and triggering of programmed cell death by leakage of cytochromes into the cytoplasm) and a shortening of the cells lifespan. The mitochondrial DNA is without protection from oxidative stress, so (the most common damaging agent is the 8OHdG {8-hydroxy-2′-deoxyguanosine} formed by oxidized guanine bases) and much more susceptible to deletions/damage. As biomechanically described above, these mutations accumulate during normal aging, the most frequent of which being the 4,977 base pair deletion known as the common deletion (significantly increased in photoaged skin). When human fibroblasts are chronically exposed to UVA irradiation, there was a time/dose dependent generation of the common deletion caused by singlet oxygen generation (free radical formation). This generation of the common deletion was diminished in the presence of singlet oxygen quenchers (antioxidants) and duplicated in non-irradiated cells by thermochemical means (deuterium oxide enhanced production of singlet oxygen) indicating a clear role in free radical modulated formation of mitochondrial deletions and prevention of same via antioxidants.

Mitochondrial deletions have been shown to be either causative agents or co-factors in many aging disorders. In Parkinson's disease clear evidence of a high burden of mitochondrial deletions in the neurons of the substantia nigra in aged individuals has been demonstrated. Mitochondrial deletions and defects have also been identified in heart disease, Alzheimer's disease, fatigue syndromes and many genetic disorders. Increased mtDNA deletions have been reported in multiple cell types, fibroblasts, retinal pigment epithelial cells, and neurons. Specifically of interest is the ratio of “clean” mtDNA to the “common deletion”, which has been shown to increase with the aging of the cell, the addition of oxidative stress and in cancerous cells. The ability of antioxidants to modulate the production of these deletions has been demonstrated to be dose dependant (lower dosages are effective in reducing the formation of the common deletion while conversely higher doses have been shown to be ineffective, and in fact are thought to act as electron donors and facilitate the production of ROS).

This theory has been supported through experimental evidence which suggests, among other things, that there are morphological differences between mitochondria in young and old cells, membrane potentials in older mitochondria are decreased, the activity level of cytochrome oxidase (COX) present in old muscle cells old cells is diminished, and that mtDNA deletions, point mutations and other changes to the structure of mitochondrial DNA increase with age.

The rate of mutation in mitochondrial DNA is as much as ten times higher than the rate of mutation in nuclear DNA. This may be due to the limited ability to repair DNA in mitochondria. Mitochondrial DNA is particularly susceptible to damage from free oxygen radicals generated during the production of ATP within the electron transport chain. ROS are produced in part when electrons get stalled on complex I or III and thus bypassing complex I with the electron transport is one mechanism to reduce ROS. Mitochondrial DNA is attached to the mitochondrial inner membrane which is a source of oxygen radicals and also it lacks protective histones making the innate repair ability more limited. This accumulated damage can make it difficult to copy the DNA, or produce deletions and mutations in the DNA. This damage over the years not only creates reduced function, but also premature aging and in some cases disease states. While the mitochondria have some ability to repair DNA, the importance of the ability to protect, defend or repair mitochondrial DNA and function can be appreciated.

Another pathway for increasing lifespan is to increase mitochondrial respiration either directly or by increasing the total overall number of mitochondria. Increasing the NAD/NADH ratio is produced with an increase in mitochondrial respiration which can be associated with the activation of PGC-1alpha which can induce mitochondrial biogenesis which increases mitochondrial numbers. Thus increasing the number of mitochondria by modulating the activity of PGC1-a is a target for lifespan altering/modulating compounds.

It is well known that oxidative stress produced by free radicals or reactive oxygen species (ROS) can produce a wide range of cellular damage which if not perfectly repaired results in cellular damage, injury, aging or apoptosis. Many small imperfectly repaired injuries accumulate over time to degrade the vital functions of cells, tissues, organs and ultimately the entire organism. This damage may occur in any of the cellular components, but of particular interest are the mitochondria which are the ‘power plants’ of living cells and which provide energy for cellular activities but which also control a process called apoptosis or programmed cell death. The mitochondria also possess their own unique DNA separate from the cell's nuclear DNA. Unlike nuclear DNA, the mitochondrial DNA has a more limited ability to repair DNA damage. Also mitochondria can actually create their own free radicals as part of normal cellular functioning. Thus mitochondria are particularly susceptible to damage from oxidative stress and the cellular damage can profoundly affect the function of the entire cell. This may result in decreased lifespan of the cells and ultimately the entire organism.

In general, increasing the chronological lifespan is related to protection from oxidative stress, minimizing DNA mutations within the mitochondria, and increasing resistance to heat shock. It has been demonstrated that increased large scale mitochondrial DNA mutations termed deletions produced by exposure to a toxic chemical (such as ethidium bromide) decreases various vital mitochondrial functions including oxygen consumption which is required to produce cellular energy or ATP. When an antioxidant was provided some of these changes were prevented or minimized suggesting that increased environmentally induced ROS production leads to altered mitochondrial gene or protein expression which may be diminished by the protective effects of certain antioxidants. Chronic oxidative stress leads to premature aging.

Some studies have shown that inducing changes in the mitochondrial DNA without oxidative stress also accelerates the aging process of cells. Chronic inflammation also may speed up the aging process. So while there are many theories of aging, it is probable that the processes involved in many of these theories play some role in the process of aging and also accelerated or premature aging. Foremost among these appear to be the role of oxidative stress and ROS and mitochondrial DNA changes (both secondary to oxidative stress as well as DNA changes independent of oxidative stress) as well as inflammation. All three of these processes may be impacted in a favorable way by various antioxidants which interact with these processes. Different antioxidants have different mechanisms of action and there are different pathways involved in ROS and oxidative stress damage. Thus the use of antioxidants to prevent premature aging and to act as an antidote to oxidative stress is well documented if not fully understood.

However, while preventing the premature aging of a cell, organ, tissue or organism generally speaking is one way to extend the lifespan, these processes are primarily methods to prevent premature shortening of the lifespan. To use humans as an example, humans are all exposed to various factors including but not limited to those just described which if we were to counteract those would effectively extend our lifespan, but this is primarily by preventing damage rather than repairing damage that shortens lifespan.

To truly extend the lifespan of a living cell—and by extension the organ, tissue or entire organism—it is beneficial to repair damage in addition to preventing damage. The genes which control the cellular repair mechanisms, if activated or enhanced in the proper way, may effectively extend the lifespan of a cell. This may take two forms: extending the lifespan of a cell which is damaged or injured by properly repairing that damage and also by causing the cell to live or replicate itself longer than it would have occurred naturally.

Cancer cells may accomplish the latter by a process termed immortalization and this may also be created in the laboratory in research conditions. Some view cancer as a form of aging as the cellular repair mechanisms have either not fully repaired damage or they have failed to kill a cell which is damaged beyond repair. One example is a type of skin cancer which is produced by injury from UVB light. UV light is known to produce changes in DNA termed thymine dimers whereby there is cross linking which occurs within the DNA and this defect or mutation produces basal cell carcinoma of the skin—a very common type of skin cancer caused by sunlight. There are also DNA repair enzymes which if they perform their job properly will repair this damage thus preventing the skin cancer from developing. In the case of basal cell skin cancer, sun light exposure leads to the formation of thymine dimers, a form of DNA damage and when the DNA repair does not remove this UV-induced damage, not all crosslinks are excised. There is, therefore, cumulative DNA damage leading to mutations. Apart from the mutagenesis, sunlight depresses the local immune system, possibly decreasing immune surveillance for new tumor cells. This risk is primarily based on UV exposure and the degree of protective pigment in the skin, but there is also a rare syndrome termed basal-cell nevus syndrome, or Gorlin's syndrome in which much less UV exposure is required because there is defective DNA repair. The cause of this syndrome is a mutation in the PTCH1 tumor-suppressor gene at chromosome 9q22.3, which inhibits the hedgehog signaling pathway ultimately leading to production of the cancer. While basal cell carcinoma of the skin is not a fatal form of cancer it does illustrate the role of environmental damage in producing cancer and also the role of genetic inheritance in making some individuals more or less likely to have this problem as well as affecting the age of onset and severity of the cancer.

If one thinks then of other cancers which destroy cellular or organ function thus limiting healthy lifespan or which lead to a fatal outcome producing a shortened lifespan, one may better appreciate that while there are many factors which lead to shortened lifespan there are also many means to diminish or avoid these factors, and that the ability to repair the damage is vital to achieving and optimal healthy lifespan.

The ability to extend or prolong lifespan (both healthy and less healthy) lies in the ability to extend the lifespan of cells, both differentiated specialized cells and also undifferentiated stem and progenitor cells so that cell lifespan is longer or so that new cells replace senescent cells which lose their function or die. A cell normally has a finite lifespan determined by the number of cell divisions which are possible. The Hayflick Limit theory discusses one view of lifespan limitations. An organ may be repopulated with cells to regenerate itself from the stem cell population but the stem and progenitor cells themselves have a finite lifespan. The ability to extend the lifespan of differentiated cells and/or stem and progenitor cells lies at the heart of extending lifespan of an organism.

The ability to repair cellular or DNA damage produced by environmental or genetic factors may extend the lifespan of a cell. The ability to extend the natural lifespan of such a cell will also extend the lifespan of a cell. Such a cell may be a differentiated cell or a stem cell. On a broader scale either or both of these events may lead to extending the lifespan of an entire organ or organism provided that some other intervening factor limits or shortens the life of the organ or organism. An exception is that extending the life of a cancerous cell or stem cell may instead shorten the life of the organ or organism and is thus undesirable. Making cancer cells mortal while making healthy cells if not immortal at least longer lasting is an important concept as longevity and tumor suppression are in some ways opposite goals. Telomerase is a critical enzyme in determining cell lifespan and its activation enables cells to overcome senescence, but also allows cancer cells to proliferate. Activity of telomerase then becomes a vital issue to consider in extending or shortening the lifespan of living cells.

The discovery of sirtuins (cellular enzymes that increase DNA repair and the production of antioxidants) and the SIRT pathway able to increase the lifespan of yeast cells with no decrease in the replicative capacity was another breakthrough in understanding aging. Sirtuins and the SIRT pathway are thought to be regulated via changes in the intracellular NAD/NADH ratio and the related energy metabolism of the mitochondria. The SIRT pathway is involved in the caloric restriction process, although the mechanism is poorly understood, it is thought primarily to revolve around the lowered instance of glycolysis that CR creates. Three of the seven mammalian sirtuins (SIRT3, 4 and 5) are targeted to mitochondria. SIRT1 itself also regulates mitochondrial activity. The activity of SIRT3 has been most clearly described and it functions in the mitochondria as an activator of special enzymes that spontaneously form NADPH the key component need for the regeneration of cellular anti stress systems (this alternate energy production pool explains why the stressful cellular event of caloric restriction seems to enhance cell longevity; by creating NADPH without the need for the primary energy metabolism of food). Resveratrol is the most potent activator of these sirtuin compounds.

The technology described herein is different from use of resveratrol and sirtuin modulation because the current technology utilizes antioxidant compounds to directly modulate the gene expression of genes/proteins and complexes vital to the maintenance of telomere length and/or mitochondrial membrane stability/free radical elimination, whereas the sirtuins and resveratrol modify the energy metabolism of the cell and boost the “anti-stress” response. Additionally, the antioxidant idebenone may help reduce ROS activity in mitochondria by helping electrons in the electron transport system bypass Complex I (where most of the ROS is formed) and donate the electrons into Complex III.

Emerging evidence suggests that microRNA (miRNA) may play a regulatory role in both aging and cancer. miRNAs appear to influence such systems as cell cycle, DNA repair, oxidative stress responses and apoptosis and have been shown to be abnormally expressed later in life. In view of this, also provided herein are methods of altering the expression of one or more of the life-span influencing genes identified herein.

There is value in extending the lifespan of a cell whether in vitro or in vivo. Protecting the cell against stress or oxidative stress responses or DNA damage and controlling the cell cycle or apoptosis or stem cell activity can potentially extend the life of the cell.

### I. Abbreviations

ANT ADP/ATP translocase

### CoA Coenzyme A

Complex I NADH ubiquinone oxidoreductase
Complex II succinate ubiquinone reductase
Complex III ubiquinone-cytochrome c oxidoreductase
Complex IV (or COX) cytochrome c oxidase
Complex V (F1/F0 ATPase) ATP synthase

### IF1 Inhibitor of F1/F0 ATPase

LC-MS/MS liquid chromatography mass spectrometry/mass spectrometry
M F1F0 mitochondrial F1/F0 ATPase
mAb monoclonal antibody
MALDI-TOF matrix assisted laser desorption/ionization time-of-flight
mtDNA mitochondrial DNA
NADH nicotinamide adenine dinucleotide
ORAC oxygen radical absorbance capacity
OD optical density

### OMIM Online Mendelian Inheritance in Man

OXPHOS oxidative phosphorylation
PDH pyruvate dehydrogenase complex
PMSF phenylmethylsulfonyl fluoride
ROS reactive oxygen species

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Addressable: Capable of being reliably and consistently located and identified, as in an addressable location on an array.

Antioxidant: A molecule or atom capable of slowing or preventing transfer of electrons from one molecule/atom to another (oxidizing agent).

Antisense, Sense, and Antigene: Double-stranded DNA (dsDNA) has two strands, a 5′->3′ strand, referred to as the plus strand, and a 3′->5′ strand (the reverse complement), referred to as the minus strand. Because RNA polymerase adds nucleic acids in a 5′->3′ direction, the minus strand of the DNA serves as the template for the RNA during transcription. Thus, the RNA formed will have a sequence complementary to the minus strand and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a dsDNA target.

Apoptosis: The process by which cells are programmed to die or lose viability. Commonly triggered by cytochrome leakage from the mitochondria and accompanied by signaling cascades (caspases and other proteins) resulting in: decreased mitochondrial and energy potential via the electron transport system, an build up of reactive oxygen species and free radical and loss of membrane integrity.

Array: An arrangement of molecules, particularly biological macromolecules (such as polypeptides or nucleic acids) or biological samples (such as tissue sections) in addressable locations on a substrate, usually a flat substrate such as a membrane, plate or slide. The array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized to such an extent that it benefits from microscopic examination for evaluation.

Within an array, each arrayed molecule (e.g., oligonucleotide) or sample (more generally, a “feature” of the array) is addressable, in that its location can be reliably and consistently determined within the at least two dimensions on the array surface. Thus, in ordered arrays the location of each feature is usually assigned to a sample at the time when it is spotted onto or otherwise applied to the array surface, and a key may be provided in order to correlate each location with the appropriate feature.

Often, ordered arrays are arranged in a symmetrical grid pattern, but samples could be arranged in other patterns (e.g., in radially distributed lines, spiral lines, or ordered clusters). Arrays are computer readable, in that a computer can be programmed to correlate a particular address on the array with information (such as identification of the arrayed sample and hybridization or binding data, including for instance signal intensity). In some examples of computer readable array formats, the individual spots on the array surface will be arranged regularly, for instance in a Cartesian grid pattern, that can be correlated to address information by a computer.

The sample application spot (or feature) on an array may assume many different shapes. Thus, though the term “spot” is used herein, it refers generally to a localized deposit of nucleic acid or other biomolecule, and is not limited to a round or substantially round region. For instance, substantially square regions of application can be used with arrays, as can be regions that are substantially rectangular (such as a slot blot-type application), or triangular, oval, irregular, and so forth. The shape of the array substrate itself is also immaterial, though it is usually substantially flat and may be rectangular or square in general shape.

Binding or interaction: An association between two substances or molecules, such as the hybridization of one nucleic acid molecule to another (or itself). Disclosed arrays are used to detect binding of, in some embodiments, a labeled nucleic acid molecule (target) to an immobilized nucleic acid molecule (probe) in one or more features of the array. A labeled target molecule “binds” to a nucleic acid molecule in a spot on an array if, after incubation of the (labeled) target molecule (usually in solution or suspension) with or on the array for a period of time (usually 5 minutes or more, for instance 10 minutes, 20 minutes, 30 minutes, 60 minutes, 90 minutes, 120 minutes or more, for instance over night or even 24 hours), a detectable amount of that molecule associates with a nucleic acid feature of the array to such an extent that it is not removed by being washed with a relatively low stringency buffer (e.g., higher salt (such as 3×SSC or higher), room temperature washes). Washing can be carried out, for instance, at room temperature, but other temperatures (either higher or lower) also can be used. Targets will bind probe nucleic acid molecules within different features on the array to different extents, based at least on sequence homology, and the term “bind” encompasses both relatively weak and relatively strong interactions. Thus, some binding will persist after the array is washed in a more stringent buffer (e.g., lower salt (such as about 0.5 to about 1.5×SSC), 55-65° C. washes).

Where the probe and target molecules are both nucleic acids, binding of the test or reference molecule to a feature on the array can be discussed in terms of the specific complementarity between the probe and the target nucleic acids. Also contemplated herein are protein-based arrays, where the probe molecules are or comprise proteins or peptides, and/or where the target molecules are or comprise proteins or peptides.

Biological Sample: Any sample that may be obtained directly or indirectly from an organism, including whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organelles (such as mitochondria), organs, and/or extracts of tissues, cells (such as, fibroblasts, peripheral blood mononuclear cells, or muscle cells), organelles (such as mitochondria) or organs. An “organism” includes, without limitation, plants, animals, or microbes. The term “animal” includes vertebrate or invertebrate animals, such as mammals (for example, humans), insects (for example, Drosophila melanogaster), nematodes (for example, Caenorhabditis elegans), and fish (for example, Danio rerio, aka, zebrafish). A biological sample may also be a laboratory research sample such as a cell culture supernatant. The sample is collected or obtained using methods well known to those skilled in the art.

Caffeic Acid (3-(3,4-Dihydroxyphenyl 3,4-Dihydroxy-cinnamic acid trans-Caffeate 3,4-Dihydroxy-trans-cinnamate) 2-propenoic acid (E)-3-(3,4-dihydroxyphenyl)-2-propenoic acid 3,4-Dihydroxybenzeneacrylicacid): Formally known as carbolic acid, this phenolic (crystalline acid compound derived from aromatic hydrocarbons) compound can be extracted from the coffee cherry and has been shown to be anti-carcinogenic, anti-inflammatory and have antioxidant properties with a chemical structure similar to cinnamic acid. It is soluble in water and alcohol. Methods for the isolation and characterization of caffeic acid are well known in the art; in addition, this compound is commercially available.

Carnosine: A natural amino acid with strong anti-oxidant properties (it helps bind and flush ionic metals from the system). Carnosine has been shown to extend the lifespan of fibroblast cells treated with the amino acid in culture up to 10 divisions past the Hayflick limit of non-treated cells. Carnosine also helps prevent the cross linking of protein and DNA molecules and preventing cell damage.

Catechin 3 gallate (CG): A minor polyphenolic constituent of green tea having antioxidant properties.

cDNA: A DNA molecule lacking internal, non-coding segments (e.g., introns) and regulatory sequences that determine transcription. By way of example, cDNA may be synthesized in the laboratory by reverse transcription from messenger RNA extracted from cells.

Cell Proliferation: The process by which there is an increase in the number of cells as a result of cell growth and division (mitotic cell division).

Cell Senescence: The process of cellular aging and loss of cell function and viability (death).

Chalcone: An aromatic ketone (chemical compound containing a carbonyl C═O group) intermediate in the biosynthesis of flavonoids that forms the central core of many biologically important compounds and has been shown to be able to block voltage dependant potassium channels.

Chlorogenic Acid (-[[3-(3,4-Dihydroxyphenyl)-1-oxo-2-propenyl]oxy]-1,4,5-trihydroxycyclohexanecarboxylic acid): A family of esters formed between certain trans cinnamic acids and quinic acid (most common individual chlorogenic acid formed from caffeic and quinic acids) and a major phenolic compound found in coffee and the cherry thereof. Chlorogenic acid has been shown to be effective in reducing free radicals (antioxidant ability) and inhibitory to the tumor formation process. Methods for the isolation and characterization of chlorogenic acid are well known in the art; in addition, this compound is commercially available.

Cocoa Bean: A fatty seed from the cacao tree; it contains substantial levels of polyphenols as well as levels of procyanidins. A cacao pod has a rough leathery rind about which varies in thickness dependent on species is filled with sweet, mucilaginous pulp that encases 30 to 50 large beans that are fairly soft and pinkish or purplish in color. It is these beans, containing cocoa butter and cocoa solids (the dried solids produce cocoa powder and the combination of the two creates chocolate in its many incarnations based on the amount of cocoa solids present. Inside the bean and pod itself are the polyphenolic and procyanidin compounds. These compounds have antioxidant anti cancer, nitric oxide (and more generally, free radical) modulatory capabilities, and can have non-steroidal anti-inflammatory effects as well. These polyphenols and procyanidins are commonly extracted from the bean by fermenting, drying and grinding the cocoa seeds.

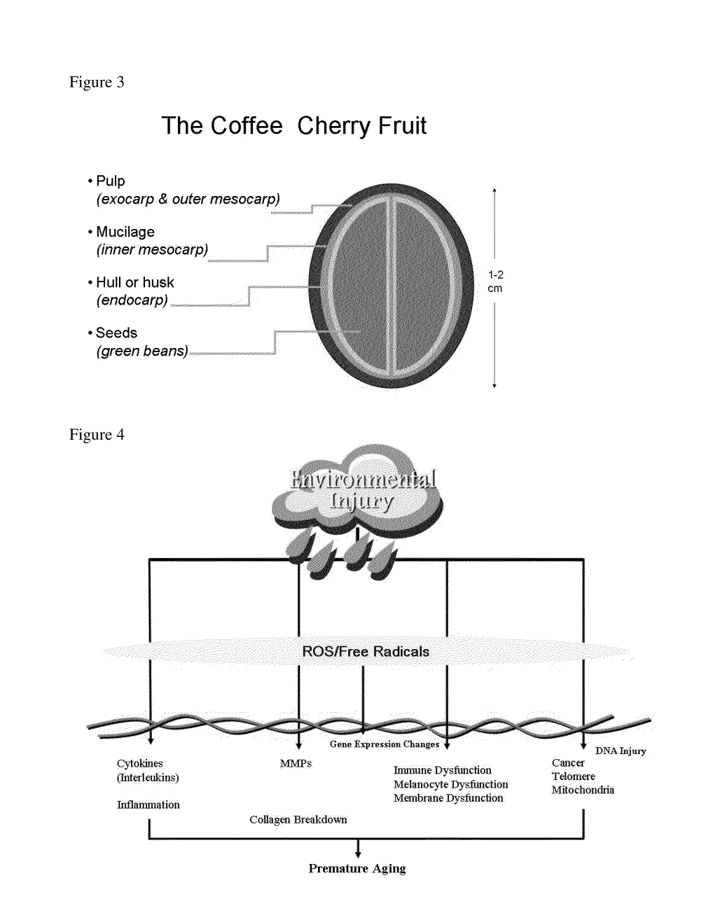

Coffee Cherry Fruit of the coffee tree Coffea rubiaceae. The pulp, husk (FIG. 3) (to a lesser degree) and mucilage of the whole coffee cherry contain high levels of polyphenols antioxidants if kept in a non fermented state and preserved. The extract of the coffee cherry is generally produced by being contacted with a solvent and will include the nutrients. Further processing of the extract (or “tea”) can allow for the purification of various aspects of the coffee cherry. One commercial producer of a coffee cherry extract is VDF FutureCeuticals, Inc. (Momence, Ill.; marketed as COFFEEBERRY®); a significant portion of their preparation is chlorogenic acid, with the other coffee acids, proanthocyanidins, etc. making up the remainder of active ingredients. By way of example, coffee cherry extract can be prepared as described previously (see, e.g., U.S. publication no. 2007/0281048 and other patent documents cited therein; U.S. Publications No. 2006/0210689, 2006/0263508, and 2009/0175973; and PCT publications no. WO 2004/098320, WO 2004/098303, WO 2006/022764 and WO 2004/098320).

Isolation of the coffee acids, including caffeic, chlorogenic, quinic and ferulic acids, as well as proanthocyanidins via (for instance) ion exchange columns and sodium acetate solutions will yield purified antioxidant components. The greatest amounts of antioxidants are found in the green coffee cherries with ripe coffee cherries having somewhat less. Polyphenols constitute a substantial portion of the active ingredients in coffee cherry extract; these polyphenols include chlorogenic acid, caffeine, caffeic acid, ferulic acid, quinic acid, and so forth. Representative analyses of different coffee cherry extracts are shown, for instance, in Table 2 of U.S. Publication Mo. 2007/0281048.

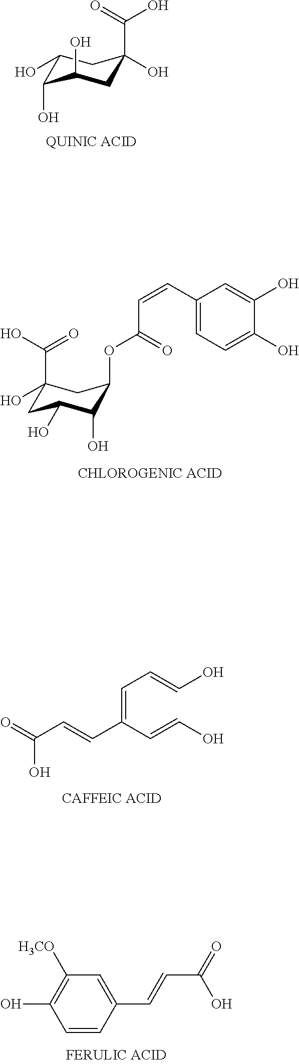

DNA (deoxyribonucleic acid): DNA is a long chain polymer that contains the genetic material of most living organisms (the genes of some viruses are made of ribonucleic acid (RNA)). The repeating units in DNA polymers are four different nucleotides, each of which includes one of the four bases (adenine, guanine, cytosine and thymine) bound to a deoxyribose sugar to which a phosphate group is attached. Triplets of nucleotides (referred to as codons) code for each amino acid in a polypeptide, or for a stop signal. The term “codon” is also used for the corresponding (and complementary) sequences of three nucleotides in the mRNA into which the DNA sequence is transcribed.

Enriched: The term “enriched” means that the concentration of a material is at least about 2, 5, 10, 100, or 1000 times its natural concentration (for example), advantageously at least 0.01% by weight. Enriched preparations of about 0.5%, 1%, 5%, 10%, and 20% by weight are also contemplated.

Enzymatic Activity: A detectable (and usually quantifiable) characteristic of at least one function of an enzyme (such as, an OXPHOS enzyme), often monitored over time or in comparison to a standard curve. Methods are well known to those of ordinary skill in the art, for detecting, determining, monitoring, and/or quantifying various enzymatic activities. Also well known are ways of using enzymatic activity assays to assess the ability of compounds (for instance, test compounds) to affect the function of the enzyme, for instance, as an inhibitor or enhancer.

For instance, “ATPase activity” is usually contemplated as the ability to detectably hydrolyze ATP. ATPase activity can be measured using various assays known to those of ordinary skill in the art, including those assays provided herein, for instance, in Example 2. In some examples, ATPase activity is measured in solution by detecting (quantitatively or qualitatively) free phosphate released by enzyme activity (such as, Complex V activity). Methods of detecting free phosphate are known and include, for example, both colorimetric and fluorescent techniques (see, e.g., Aggeler et al., J. Biol. Chem., 277:33906-33912, 2002). In other examples, ATPase activity of an immobilized enzyme (for instance, Complex V immunocaptured on a dipstick) is detected, for example, by fluorescent techniques (for example, fluorescence-based assays for free phosphate as provided by Molecular Probes, Inc., or by direct application of tissue based histochemical techniques (see, e.g., Bancroft and Stevens, Theory and Practice of Histological Techniques, 4th edition, London:Churchill-Livinstone, 1996) or slight modifications thereof, for example to account for the physical handling differences of tissue sections as compared to dipsticks.

“Oxidoreductase activity” is the ability of an enzyme to reversibly oxidize (remove protons and electrons, or reducing equivalents from) a first substrate molecule and contemporaneously reduce (add protons and electrons, or reducing equivalents to) a second substrate molecule. First and second substrate molecules typically are, but need not be, proteins, carbohydrates, lipids, or small co-factors.

Oxidation and/or reduction can be detected by any method known in the art. In some examples, a detectable change in a physical property of the oxidized and/or reduced substrate molecule(s) is measured; for example, a change in optical density (OD) at some defined wavelength. In particular examples, OD₃₄₀ can be used to monitor the ratio of NAD/NADH redox (such as, in assays of Complex I activity), or OD₆₀₀ can be used to monitor reduction of 2,6-dichlorophenolindophenol (such as, in assays for Complex II activity), or OD₅₅₀ can be used to monitor oxidation of cytochrome c (II) (such as, in assays for Complex IV activity) (see, e.g., Birch-Machin and Turnbull, Meth. Cell Biol., 65:97-117, 2001). In other examples, oxidation and/or reduction can be detected by monitoring a change in the properties of a prosthetic group in the oxidoreductase enzyme; for example, the ratio of OD₆₀₅/OD₆₃₀ can be used to monitor heme aa3 of Complex IV (see, e.g., Rickwood et al., in Mitochondria. A Practical Approach, ed. by Darley-Usmar et al., Oxford:IRL Press, 1987). In still other examples, oxidation and/or reduction can be detected by coupling the oxidation or reduction reaction of interest to another more easily monitored redox reaction, such as oxidation or reduction of a chromogenic (Birch-Machin and Turnbull, Meth. Cell Biol., 65:97-117, 2001) or fluorogenic (Molecular Probes, Inc.) substrate.

“Reductase activity” is the ability of an enzyme to reduce (add electrons to) a substrate molecule, which typically is, but need not be, a protein, a carbohydrate, a lipid or a small co-factor. The reducing equivalents are obtained by the enzyme from some other molecule which is thereby oxidized either contemporaneously with, or at some time prior to, the reductase enzyme/substrate reaction. Reductase activity can be measured using various assays known to those of ordinary skill in the art. For example, assays for activity of Complex II can follow reduction of the oxidized substrate 2,6-dichlorophenolindophenol by monitoring changes in OD600 (Birch-Machin & Turnbull, Meth. Cell Biology, 65:97-117, 2001).

“Oxidase activity” is the ability of an enzyme to oxidize (remove protons and electrons, or reducing equivalents, from) a substrate molecule, which typically is, but need not be, a carbohydrate, a lipid or a small co-factor. The reducing equivalents are typically transferred by the enzyme to some other molecule which is thereby reduced either contemporaneously with, or at some time after, the oxidase enzyme/substrate reaction. Oxidase activity can be measured using various assays known to those of ordinary skill in the art. For instance, Complex IV oxidase activity can be detected by observing the oxidation of cytochrome c by measuring OD₅₅₀ (Birch-Machin and Turnbull, Meth. Cell Biol., 65:97-117, 2001).

Epigallocatechin gallate (EGCG): The most abundant of the antioxidant catechins found in green tea. It is an ester of epigallocatechol and gallic acid.

Epicatechin gallate (ECG): A polyphenol found in green tea and having antioxidant properties.

Ester: A class of chemical compound that consists of an acid that has at least one —OH (hydroxyl) group replaced by an —O-alkyl (alkoxy) group.

Ferulic Acid ((E)-3-(4-hydroxy-3-methoxy-phenyl)prop-2-enoic acid): A compound serving as a precursor for other aromatic compounds, it is found most commonly in the plant cell walls where it associates with dihydroferulic acid, to facilitate the crosslinking of lignin and polysaccharides conferring rigidity to the cell wall. It can be found in coffee cherry, has antioxidant activity and is biologically synthesized by methylation of caffeic acid. Methods for the isolation and characterization of ferulic acid are well known in the art; in addition, this compound is commercially available.

Free Radical: Any atom or molecule having a single, unpaired electron in an outer shell.

FOXO1, 3 and 4 (Forkhead Box O1A, O3A, and O4A): Activation of serine threonine kinase which inactivates apoptotic machinery. Overexpression causes growth suppression in a of cell lines variety.

Gallocatechin gallate (GCG): A member of antioxidant polyphenols found in green tea.

Gnetin H: A stilbene (a hydrocarbon with a trans ethane double bond substituted with a phenyl group on both carbon atoms of the double bond) resveratrol derivative from peony seeds having antioxidant properties and mimicking the effects of resveratrol.

Golgi apparatus: A cell organelle involved in the processing and packaging of proteins and lipids produced by and/or moved through a cell.

Hayflick Limit: The number of times a cell can undergo mitosis before the telomeres are shortened to a critical length and the cell begins to senesce. Each mitosis event decreases the length of the telomere and pushes the “aging” cell towards senescence. This limit is thought to be a mechanism through which the body can control cancerous cell growth; since the more times a cell undergoes mitosis the more chances for a problematic mutation or transcription error to occur.

Healthy longevity: The concept of having entire organisms (as well as organs, tissues and individual cells) at optimal genetic and functional health. While not limited to these issues, this means for example that the DNA is not significantly damaged or mutated and is in a state comparable to the configuration that would occur in a natural healthy infant or fetus. In other embodiments, the DNA has been altered to be equivalent or better than that status through, e.g., repair or genetic engineering. Similarly, in some embodiments the mitochondrial number and/or function and/or respiratory efficiency are similarly optimal or supra optimal. Metabolic pathways and immune function also may be likewise optimized, and existing environmental damage may have been repaired. Intrinsic chronologic aging and/or oxidative stress damage from normal cellular processes such as free radical damage within mitochondria have also been mitigated or reversed or repaired or otherwise restored to a youthful optimally functional status or a close approximation of the same. Unhealthy cells, including even cancerous cells, which have not been repaired, are eliminated via apoptosis or the death of these cells has been modulated to be accelerated. Significantly gene expression patterns and pathways have been reregulated, or reset or resignalled in such a fashion as to optimize the function and health of the cells and by extension the tissues, organs and organisms that these cells comprise. One end result of at least one or perhaps more of these processes is that the cells achieve maximal longevity or lifespan and/or function optimally or at maximal efficiency and effectiveness for the duration of their lifespan. Understanding that such a process may not be undertaken until substantial damage from aging, disease, diet, injury, environmental exposure, medication or medical therapy side effects, etc. it is understood that even a partial achievement of one or more of these goals would improve the length of the lifespan or make the remaining lifespan duration healthier. Modulating cell function to achieve one or more of these goals is then a means of producing a state termed healthy longevity. The modulation of cell activity to accomplish this may involve in some instances modulating to kill cells prematurely and in a manner diminish the cells health to the point of cell death in order to remove unhealthy cells which may harm the tissue, organ or organism or even which may stimulate the creation and replacement of the unhealthy or sub-optimally healthy cell(s) with new cells via cell division of healthy cells, biogenesis of new cells or replacement of cells via stem cells or autologous transplant or allograft or other types of transplanted cells including genetically engineered cells for transplantation. The treatment of such cells with the process of this invention prior to or after transplantation is also envisioned as a means to produce healthy longevity in these ‘new’ cells.

High throughput genomics: Application of genomic or genetic data or analysis techniques that use arrays, microarrays or other genomic technologies to rapidly identify large numbers of genes or proteins, or distinguish their structure, expression or function from normal or abnormal cells or tissues, or from cells or tissues of subjects with known or unknown phenotype and/or genotype.

Histone(s): Lysine and Arginine rich, basic proteins associated with DNA in eukaryotic chromosomes resembling “beads on a string”. These proteins form the scaffold which the DNA wraps around to form the chromatin structure.

HPGD (Hydroxyprostaglandin Dehydrogenase): Involved in many cellular processes specifically inflammation. As an NAD dependant dehydrogenase, HPGD is the primary prostaglandin degrading enzyme.

HSPA1A (Heat Shock 70-KD Protein 1A): Ubiquitous highly conserved protein involved in many functions. HSPA1A is thought to be proliferative, when expressed, for cancer cells, involved in apoptosis and regulation of acute stress.

HSPA1B (Heat Shock 70-KD Protein 1B): A “stress” protein expressed in response to heat, oxidative damage, free radicals and toxic metal ions. Structurally and functionally comparable to other heat shock proteins (varying in their inducibility due to stress) and involved in the cell response to damage/stress.

HSPA1L (Heat Shock 70-KD Protein 1 L): A “stress” protein expressed in response to heat, oxidative damage, free radicals and toxic metal ions. Structurally and functionally comparable to other heat shock proteins (varying in their inducibility due to stress) and involved in the cell response to damage/stress.

Human Cells: Cells obtained from a member of the species Homo sapiens. The cells can be obtained from any source, for example peripheral blood, urine, saliva, tissue biopsy, skin scrape, surgical specimen, amniocentesis samples and autopsy material. From these cells, biological components such as genomic or mitochondrial DNA, mRNA (from which one can make cDNA), RNA, and/or protein can be isolated.

Hybridization: Nucleic acid molecules that are complementary to each other hybridize by hydrogen bonding, which includes Watson-Crick, Hoogsteen or reversed Hoogsteen hydrogen bonding between complementary nucleotide units. For example, adenine and thymine are complementary nucleobases that pair through formation of hydrogen bonds. “Complementary” refers to sequence complementarity between two nucleotide units. For example, if a nucleotide unit at a certain position of an oligonucleotide is capable of hydrogen bonding with a nucleotide unit at the same position of a DNA or RNA molecule, then the oligonucleotides are complementary to each other at that position. The oligonucleotide and the DNA or RNA are complementary to each other when a sufficient number of corresponding positions in each molecule are occupied by nucleotide units which can hydrogen bond with each other.

“Specifically hybridizable” and “complementary” are terms that indicate a sufficient degree of complementarity such that stable and specific binding occurs between the oligonucleotide and the DNA or RNA or PNA target. An oligonucleotide need not be 100% complementary to its target nucleic acid sequence to be specifically hybridizable. An oligonucleotide is specifically hybridizable when binding of the oligonucleotide to the target DNA or RNA molecule interferes with the normal function of the target DNA or RNA, and there is a sufficient degree of complementarity to avoid non-specific binding of the oligonucleotide to non-target sequences under conditions in which specific binding is desired, for example under physiological conditions in the case of in vivo assays, or under conditions in which the assays are performed.

Hybridization conditions resulting in particular degrees of stringency will vary depending upon the nature of the hybridization method of choice and the composition and length of the hybridizing DNA used. Generally, the temperature of hybridization and the ionic strength (especially the Na concentration) of the hybridization buffer will determine the stringency of hybridization. Calculations regarding hybridization conditions required for attaining particular degrees of stringency are discussed by Sambrook et al. in Molecular Cloning: A Laboratory Manual, Cold Spring Harbor Laboratory Press (1989), chapters 9 and 11, herein incorporated by reference.

Idebenone (6-(10-hydroxydecyl)-2,3-dimethoxy-5-methyl-1,4-benzoquin-one): Reference German patent document DE3049039, European patent 788793, and U.S. Pat. Nos. 4,436,753, 5,059,627, 5916925, application 20050152857 and WIPO 9907355 to describe the use of oral, parenteral or percutaneous preparations of idebenone or derivatives for the treatment of dementia, circulatory disease induction of neural growth factors and resistance to sunburn cell formation. Methods for the isolation and characterization of idebenone are well known in the art; in addition, this compound is broadly commercially available. Idebenone is a synthetic molecule that does not occur in nature and mimics the structure and function of ubiquinone and ubiquinol with similar results for Redox potential and free radical quenching capabilities.

Idebenone has also been shown via chemiluminescence to intercept the pro-oxidative effect of tocopherol oxidation products occurring after 24 hours. In the measurements of the lipid hydroperoxides generated as a result of the oxidation of lipids due to, for example, UV radiation or free radical damage, idebenone was shown to have the highest reduction of said products of the tested antioxidants (U.S. Pat. No. 6,756,045).

Idebenone (chemical) derivative: Derivatives of idebenone may also be suitable for use methods described herein, including the maintenance of telomere length and increase in the longevity of cellular lifespan. Such derivatives may include the salts and/or esters of idebenone, protein bound forms or other derivatives. Examples of idebenone derivatives include esters of idebenone where idebenone is esterified using glycosaminoglycans (GAGS), and/or their salts, for example HA (hyaluronic acid) having a molecular weight of 1 to 1,000,000 and its salts of hyaluronidase inhibitors like inter-alpha-trypsin inhibitor. An example of a hydrophilic idebenone ester is idebenone sulphonic acid.

IDH2 (Isocitrate Dehydrogenase 2): NADP dependant isocitrate dehydrogenase that is responsible for playing a major role in mitochondrial redox balance and mitigating damage by oxidative stress by providing NADPH for NADPH dependent antioxidant enzymes.

IFI44 (Interferon Induced Protein 44): Interferon stimulated response element induced by interferon alpha and bets, but not gamma possibly produced in response to viral induction.

In vitro amplification: Techniques that increase the number of copies of a nucleic acid molecule in a sample or specimen. An example of in vitro amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid.

The product of in vitro amplification may be characterized by electrophoresis, restriction endonuclease cleavage patterns, oligonucleotide hybridization or ligation, and/or nucleic acid sequencing, using standard techniques.

Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Keratinocyte: A cell type comprising 95% of the epidermis and producer of the structural protein(s) keratin(s).

KL (Klotho): Reduced expression of KL is thought to be a causative factor in many degenerative processes including, arteriosclerosis, osteoporosis, skin atrophy and general aging. KL functions by converting members of the Fibroblast Growth Factor/FGFR family and mediating trans-epithelial calcium transport and metabolism. This effect on cellular calcium levels may tie in to apoptosis and membrane potentials.

KU70 (Thyroid Autoantigen, 70 kD; G22P1): Part of a cell cycle dependant (associated with the chromosomes in interphase and dissociated in prophase) dsDNA binding complex with a proposed role in DNA repair or transposition.

Label: Any molecule or composition bound to an analyte, analyte, detector reagent, analog or binding partner that is detectable by spectroscopic, photochemical, biochemical, immunochemical, electrical, optical or chemical means. Non-limiting examples of labels include enzymes, colloidal gold particles, colored latex particles, radioactive isotopes, enzyme substrates, co-factors, ligands, chemiluminescent or fluorescent agents, haptens, protein-adsorbed silver particles, protein-adsorbed iron particles, protein-adsorbed copper particles, protein-adsorbed selenium particles, protein-adsorbed sulphur particles, protein-adsorbed tellurium particles, protein-adsorbed carbon particles, and protein-coupled dye sacs. Methods for labeling and guidance in the choice of labels appropriate for various purposes are discussed, e.g., in Sambrook et al., Molecular Cloning: A Laboratory Manual, CSHL, New York, 1989 and Ausubel et al., Current Protocols in Molecular Biology, Greene Publ. Assoc. and Wiley-Intersciences, 1998. The attachment of a compound (e.g., an antibody) to a label can be through covalent bonds, adsorption processes, hydrophobic and/or electrostatic bonds, as in chelates and the like, or combinations of these bonds and interactions and/or may involve a linking group.

Specific example detectable labels suitable for conjugating to antibodies, including antibodies used in high throughput screening systems, include radiolabels and other detectable molecules linked to the antibodies using various chemical linking groups or bifunctional peptide linkers. A terminal hydroxyl can be esterified with inorganic acids, e.g., ³²P phosphate, or ¹⁴C organic acids, or else esterified to provide linking groups to the label. Enzymes of interest as detectable labels will primarily be hydrolases, particularly esterases and glycosidases, or oxidoreductases, particularly peroxidases. Fluorescent compounds include fluorescein and its derivatives, rhodamine and its derivatives, dansyl, umbelliferone, and so forth. Chemiluminescers include luciferin, and 2,3-dihydrophthalazinediones (e.g., luminol), and the like.

Langerhans cell: (Dendritic) cells found in the epidermis and lymph nodes responsible for the capture, uptake and processing of antigens and foreign particles in the skin.

Lifespan: The length of time a cell, tissue or organism remains viable. There are 2 components to this the Potential (or Inherent) Lifespan defined as the unaltered lifespan of the cell or organism based solely on genetic factors and the Observed Lifespan defined as the length of time the cell or organism will remain viable when all damaging (Oxidative Stress, Poor Nutrition) stimuli are factored in.

Liposome or liposomal: An aqueous compartment or pocket, often microscopic, enclosed by a bimolecular phospholipid membrane; a lipid vesicle. Liposomes have been exploited to deliver compounds and compositions, for instance cells; when the liposome comes in contact with another membrane (e.g., a cell membrane), the two membranes fuse and the encapsulated liposomal contents are released into the cell. This effectively transports the aqueous contents trapped in the liposome across and into the contacted membrane-bound compartment (e.g., cell). Means of preparing liposomes are well known to those of skill in the art. See, e.g., Betageri et al., Liposome Drug Delivery Systems, Technomic Publishing Co., Inc., Lancaster, Pa. (1993).

LMNA (Lamin A/C): Gene that codes for structural components of the protein network that determines the size and shape of the nucleus. An intermediate filament thought to be involved in Hutchinson-Guilford progeria syndrome.

Melanocyte: A pigment producing cell that provides color to skin, hair and eyes. It is most commonly found in the bottom layer of the skins epidermis and mid-layer of the eye.

MDH1 (Soluble Malate Dehydrogenase 1): Catalyzes a reversible reaction in the citric acid cycle (L-malate+NAD to form NADH+oxaloacetate). MDH1 is located on the same chromosome as IDH.

MDH2 (Mitochondrial Malate Dehydrogenase): Mitochondrial bound dehydrogenase. MDH2 catalyzes a reversible reaction in the citric acid cycle (L-malate+NAD to form NADH+oxaloacetate).

ME1 (Malic Enzyme 1): NADP+dependent enzyme link between the citric acid cycle and glycolytic pathway that catalyzes the reversible oxidative decarboxylation of malate to form pyruvate, CO₂ and NADPH.

ME2 (Malic Enzyme 2): Mitochondrial enzyme determined by nuclear genes, similar to ME1.

ME3 (Malic Enzyme 3): NADP+dependent mitochondrial enzymes, catalyzes the oxidative decarboxylation of malate to pyruvate using NAD+ or NADP+ as cofactors.

Meristematic: The quality of being undifferentiated or progenitor cell like, and can apply to both cells and tissues.

Merkel cell: Large oval cells found in the epidermis of vertebrates and associated with the sense of touch.

Mitochondrion (mitochondria): The small, membrane lined organelle providing most of the cells chemical energy through the electron transport systems production of adenosine triphosphate. The mitochondria are also involved in cell growth, cellular signaling, cell cycle regulation, apoptosis, and cellular differentiation. The loss of mitochondrial membrane potentials/functions and deletions of the mtDNA are also thought to be key events in the aging of cells.

Mitochondrial Biogenesis: The process by which new mitochondria are formed during the lifespan of the cell.

Mitochondrial Damage: any physical alteration in mitochondrial components, including mtDNA, proteins (such as, one or more OXPHOS proteins), or lipids, that alters mitochondrial function in a way that is detrimental to cell physiology, growth or faithful replication.

Mitochondrial Disorder: A disease resulting from altered mitochondrial function, caused by any alteration or combination of alterations of mitochondrial components (for instance, mitochondrial protein (such as, one or more OXPHOS proteins), mtDNA, or lipid) caused by genetic and/or environmental factors, including autotoxicity caused by normal cellular metabolic processes. “Late onset mitochondrial disorder” or “late onset disease” means such diseases as late onset diabetes (Diabetes Type I), Huntington's, Parkinson's and Alzheimer's diseases, ALS (amyotrophic lateral sclerosis), Schizophrenia and the like, wherein the subject is free of the disease in early life, but develops the disease during puberty or thereafter, sometimes as late as age 70 or 80.

MTND5 (NADH Dehydrogenase Subunit 5): 1 of 7 of the mitochondrial subunits of the respiratory complex. Complex I (of which subunit 5 is a part) accepts electrons from NADH and transfers them to ubiquinone and uses the energy released to drive protons across the inner mitochondrial membrane.

MTHD1 (Methylenetetrahydrofolate Dehydrogenase 1): Encodes trifunctional protein in eukaryotes that is involved in the conversion of 1-carbon derivatives into substrates for methionine and purine synthesis.

MTHFD1L (Methylenetetrahydrofolate Dehydrogenase, NADP+Dependent 1 Like): Localized to the mitochondria and involved in THF (tetrahydrofolate) synthesis therein. Involved in synthesis of purines and thymidylate; supporting cellular methylation reactions.

MTHFR (5-10, Methylenetetrahydrofolate Reductase): Catalyzes the formation of a substrate for remethylating methionine.

NADK (NAD Kinase): Catalyzes formation of NADP which is reduced to act as an electron donor for various biochemical reactions.

NADSYN1 (NAD Synthetase 1): Responsible for the final step in the formation of NAD, a coenzyme in redox reactions, a substrate for posttranslational modifications and a common cell signaling mechanism.

NDUFA2, 3, 4, 4L2, 5, 6, 7, 9, 10 and 12 (NADH-Ubiquinone Oxidoreductase 1 alpha, subcomplexes 2, 3, 4, 4L2, 5, 6, 7, 9, 10 and 12): Genes coding for the various subcomplexes that compose the first and largest complex in the respiratory chain (Complex I). Complex I is responsible for NADH oxidation, ubiquinone reduction and proton ejection from the mitochondria.

NDUFB2, 3, 5, 6, 7, 8, and 9 (NADH-Ubiquinone Oxidoreductase 1 beta, subcomplexes 2, 3, 5, 6, 7, 8 and 9): Genes coding for the various subcomplexes in the hydrophilic region of the first and largest complex in the respiratory chain (Complex I). Complex I is responsible for NADH oxidation, ubiquinone reduction and proton ejection from the mitochondria.

NDUFC2 (NADH-Ubiquinone Oxidoreductase 1 subunit c2): Gene coding for the subunit C2 of the first and largest complex in the respiratory chain (Complex I). Complex I is responsible for NADH oxidation, ubiquinone reduction and proton ejection from the mitochondria.

NDUFS2, 4, 5, 7, and 8 (NADH-Ubiquinone Oxidoreductase Fe—S Proteins 2, 4, 5, 7, and 8): Genes coding for the iron sulfur protein (IP) fraction of the first and largest complex in the respiratory chain (Complex I). Complex I is responsible for NADH oxidation, ubiquinone reduction and proton ejection from the mitochondria.

NDUFV2 and 3 (NADH-Ubiquinone Oxidoreductase Flavoprotein 2 and 3): Genes coding for 24 kD fraction of the first and largest complex in the respiratory chain (Complex I). Complex I is responsible for NADH oxidation, ubiquinone reduction and proton ejection from the mitochondria.

NFKB1 (Nuclear Factor Kappa B; Subunit 1): Gene that encodes for protein involved in the inflammatory process and responsible for the induction of many inflammatory proteins. Inhibition of NFKB1 has been shown to lead to delayed cell growth, apoptosis and inappropriate immune cell development.

NHP2L1 (Non-Histone Chromosome Protein 2, S. Cerevisiae, Homolog Like 1): Component of the spliceosome complex required for activation of the complex. The spliceosome is involved in removing introns from a transcribed pre-RNA segment.

NOX1, 3, 4 and 5 (NADPH Oxidase 1, 3, 4 and 5): Associated with the plasma membrane of multiple cell types, these NADPH oxidases aid the production of superoxide by a 1-electron reduction of oxygen with NADPH as the electron donor.

NOXA1 (NADPH Oxidase Activator 1): Activates (more effective with NOX) the various NADPH oxidases that generate reactive oxygen species (ROS).

NOXO1 (NADPH Oxidase Organizer 1): Responsible for targeting NOX activators to NOX and relocating NOX to subcellular compartments.

NRF1 (Nuclear Respiratory Factor 1): A transcription factor that codes for respiratory subunits and components of the mitochondrial transcription and replication machinery, which allows for the transcription of mitochondrial DNA.

NRF2 (Nuclear Factor Erythroid 2 Like 2) A gene coding for a family of leucine zipper transcription factors with some highly conserved regions with FOS and JUN. Regulates transcription of SSAT gene and aids in protein interactions.

NQO1 (NAD{P}H Dehydrogenase Quinone 1): A two-electron reductase involved in the detoxification of quinones and protection against benzene metabolites.

Nucleic acid: A deoxyribonucleotide or ribonucleotide polymer in either single or double stranded form, and unless otherwise limited, encompassing known analogues of natural nucleotides that hybridize to nucleic acids in a manner similar to naturally occurring nucleotides.

Nucleic acid array: An arrangement of nucleic acids (such as DNA or RNA) in assigned locations on a matrix, such as that found in cDNA arrays, or oligonucleotide arrays.

Nucleic acid molecules representing genes: Any nucleic acid, for example DNA (intron or exon or both), cDNA or RNA, of any length suitable for use as a primer (e.g., for in vitro amplification), probe or other indicator molecule, and that is informative about the corresponding gene.

Nucleotide: A grouping of a phosphate, a sugar and a nitrogenous base that form the structures of RNA and DNA (the RNA or DNA is determined by which sugar, ribose or deoxyribose, is involved in the grouping). “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: A linear single-stranded polynucleotide sequence ranging in length from 2 to about 5,000 bases, for example a polynucleotide (such as DNA or RNA) which is at least 6 nucleotides, for example at least 10, 12, 15, 18, 20, 25, 50, 100, 200, 1,000, or even 5,000 nucleotides long. Oligonucleotides are often synthetic but can also be produced from naturally occurring polynucleotides.

An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include PNA molecules. Such analog molecules may also bind to or interact with polypeptides or proteins.

Oxidative Stress: An imbalance within the cell, tissue or organism which results in a diminished ability to: reduce (or detoxify) biological reactive chemical intermediates, repair the damage caused by reactive chemical intermediates, or maintain the cellular reduction potential most often resulting in apoptosis.

PARP1 (Poly ADP Ribose Polymerase 1): Chromatin associated enzyme that may signal altered metabolic conditions to the chromatin. NAD dependent, post translational, ADP ribosylation plays a role in DNA repair (strand breaks, etc.) and recovery of cells from DNA damage. PARP1 activation is required for translation of apoptosis inducing factor from the mitochondria to the nucleus (required in PARP1 dependant cell death). PARP1 possibly plays a role in many other cellular types and functions (spindle cell formation, neurons, and gene targeting to list a few).

PARP2 (Poly ADP Ribose Polymerase 2): An ADP ribosyltransferase that is activated as an early cellular response to DNA strand breaks. This class of enzymes modifies nuclear proteins by ADP-ribosylation which is required for DNA repair, regulation of apoptosis and maintaining genome stability.

PGC-1 Alpha (Peroxisome Proliferator-Activated Receptor-Gamma, Coactivator 1, Alpha; PPARGC1A): A transcription coactivator of nuclear receptors which greatly increases the transcriptional activity of PPAR gamma, thyroid hormone receptor, activates the expression of key enzymes in the respiratory chain, increases the cellular content of mitochondrial DNA and stimulates mitochondrial biogenesis.

POLB (DNA Polymerase Beta): Another DNA polymerase which is primarily responsible for the base excision repair required for DNA maintenance, replication, recombination and drug resistance in eukaryotic cells.

POLD3 (DNA Directed Polymerase Delta 3): A portion of the DNA polymerase delta complex (along with PCNA, POLD1, 2, and 4) involved in replication and repair.

POLE (DNA Polymerase Epsilon): Nuclear polymerase (1 of 4) in eukaryotic cells responsible for DNA repair and replication of chromosomal DNA.

POLG (DNA Polymerase Gamma): An enzyme present in both the nucleus and mitochondria and plating a role in mitochondrial replication. A “proof reading” enzyme that increases the fidelity of mitochondrial replication and transcription.

POLI (DNA Polymerase Iota): Crystal structured human DNA polymerase that binds to an incoming nucleotide and template primer. POLI assists in bypassing DNA damage by incorporating deoxynucleotides directly across from DNA lesions.

POLL (DNA Polymerase Lambda): On of the many DNA polymerases in humans that contributes to both replication of the entire genome and the DNA repair process (telomere mediated).

Polymorphism(s): The difference in DNA sequences among a population for the same gene. Generally there are two or more alternative forms of a gene (which has changes in the nucleotide sequence) that may be harmless or associated with a diseases state. This correlation to possible disease states has made tracking and identifying polymorphisms a possible method to determine causative mutations for said disease states.

Polyphenols (some of which may be referred to as Tea Derived Antioxidants): Ester bond containing polyphenols like EGCG (epigallocatechin-3-gallate), EGC (epigallocatechin), ECG (epicatechin-3-gallate), EC (epicatechin), GCG (gallocatechin gallate), GC (gallocatechin), C (catechin) and/or CG (catechin gallate) can be used to extend the lifespan of living cells through direct influence over the gene expression of the telomere length maintenance unit and related proteins. This extension or preservation of the length of the telomere will increase the replicative capacity and time until apoptosis in living cells resulting in a prolonged duration of cell “health” and viability. Methods for the isolation and characterization of polyphenols are well known in the art; in addition, various purified polyphenols are commercially available.

POT1 (Protection of Telomeres 1): Codes for a widely conserved protein (across eukaryotes) which binds a G rich strand of telomeric DNA and protects chromosome ends.

PPARG (Peroxisome Proliferator Activated Protein Gamma): Member of the nuclear hormone receptor subfamily of transcription factors. PPARs form heterodimers with members of the retinoid receptor family and these structures regulate transcription/activation of a variety of genes. Specifically, PPARG is thought to be involved in adipocyte differentiation, proinsulin biosynthesis, insulin release and activation of inflammatory response genes.

Proanthocyanidins (Oligomeric Proanthocyanidin; OPC): A class of flavonoids (plant secondary metabolic products including catechins) most commonly found in many plants, with the extracting into wine being the most common occurrence. They area also found in coffee cherry, and extracts made therefrom, and have been shown to be able to absorb many oxygen free radicals. Methods for the isolation and characterization of proanthocyanidins are well known in the art; in addition, specific proanthocyanidin compounds are commercially available.

Probes and primers: Nucleic acid probes and primers can be readily prepared based on the nucleic acid molecules provided as indicators of taste reception or likely taste reception. It is also appropriate to generate probes and primers based on fragments or portions of these nucleic acid molecules, particularly in order to distinguish between and among different alleles and haplotypes within a single gene. Also appropriate are probes and primers specific for the reverse complement of these sequences, as well as probes and primers to 5′ or 3′ regions.

A probe comprises an isolated nucleic acid attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, e.g., in Sambrook et al. (In Molecular Cloning: A Laboratory Manual, CSHL, New York, 1989) and Ausubel et al. (In Current Protocols in Molecular Biology, John Wiley & Sons, New York, 1998).

Primers are short nucleic acid molecules, for instance DNA oligonucleotides 10 nucleotides or more in length. Longer DNA oligonucleotides may be about 15, 20, 25, 30 or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the polymerase chain reaction (PCR) or other in vitro nucleic-acid amplification methods known in the art.

Methods for preparing and using nucleic acid probes and primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). Amplification primer pairs (for instance, for use with polymerase chain reaction amplification) can be derived from a known sequence such as any of the bitter taste receptor sequences and specific alleles thereof described herein, for example, by using computer programs intended for that purpose such as PRIMER (Version 0.5, © 1991, Whitehead Institute for Biomedical Research, Cambridge, Mass.).

One of ordinary skill in the art will appreciate that the specificity of a particular probe or primer increases with its length. Thus, for example, a primer comprising 30 consecutive nucleotides of a bitter taste receptor protein encoding nucleotide will anneal to a target sequence, such as homolog of a designated taste receptor protein, with a higher specificity than a corresponding primer of only 15 nucleotides. Thus, in order to obtain greater specificity, probes and primers can be selected that comprise at least 20, 23, 25, 30, 35, 40, 45, 50 or more consecutive nucleotides of a taste receptor gene.

Also provided are isolated nucleic acid molecules that comprise specified lengths of bitter taste receptor-encoding nucleotide sequences. Such molecules may comprise at least 10, 15, 20, 23, 25, 30, 35, 40, 45 or 50 or more (e.g., at least 100, 150, 200, 250, 300 and so forth) consecutive nucleotides of these sequences or more. These molecules may be obtained from any region of the disclosed sequences (e.g., a specified nucleic acid may be apportioned into halves or quarters based on sequence length, and isolated nucleic acid molecules may be derived from the first or second halves of the molecules, or any of the four quarters, etc.). A cDNA or other encoding sequence also can be divided into smaller regions, e.g. about eighths, sixteenths, twentieths, fiftieths, and so forth, with similar effect.

Another mode of division, provided by way of example, is to divide a bitter taste receptor sequence based on the regions of the sequence that are relatively more or less homologous to other bitter taste receptor sequences.

Nucleic acid molecules may be selected that comprise at least 10, 15, 20, 25, 30, 35, 40, 50, 100, 150, 200, 250, 300 or more consecutive nucleotides of any of these or other portions of a bitter taste receptor nucleic acid molecule or a specific allele thereof, such as those disclosed herein. Thus, representative nucleic acid molecules might comprise at least 10 consecutive nucleotides of a sequence listed in DATA TABLE 7 or Array 2.

Procyanidins: Tannic (polyphenols compounds that bind or shrink proteins) compounds found in plants and especially tea and grape seed. Procyanidins are commonly associated with the bitter, astringent taste of wine. The compounds also have a very high antioxidant capacity. Methods for the isolation and characterization of procyanidins are well known in the art; in addition, certain procyanidins are commercially available.

PTOP (ACD, Mouse Homolog of; ACD): Gene responsible for targeting POT1 to telomeres permitting telomere extension. PTOP binds both POT1 and TIN2 to the TRF1 complex.

Purified: The term purified does not require absolute purity; rather, it is intended as a relative term. Thus, for example, a purified nucleic acid preparation is one in which the specified protein is more enriched than the nucleic acid is in its generative environment, for instance within a cell or in a biochemical reaction chamber. A preparation of substantially pure nucleic acid may be purified such that the desired nucleic acid represents at least 50% of the total nucleic acid content of the preparation. In certain embodiments, a substantially pure nucleic acid will represent at least 60%, at least 70%, at least 80%, at least 85%, at least 90%, or at least 95% or more of the total nucleic acid content of the preparation.

Quinic Acid (1S,3R,4S,5R)-1,3,4,5-tetrahydroxy-cyclohexanecarboxylic acid: Discovered in the 1800s, this crystalline acid compound is formed synthetically by hydrolysis of chlorogenic acid, but is found naturally in the coffee cherry. Thought to provide the “acidity” of coffee, this compound, aside from the usual antioxidant capabilities of the other coffee cherry acids, is a versatile starting compound for the synthesis of new synthetic compounds as well. Methods for the isolation and characterization of quinic acid are well known in the art; in addition, this compound is commercially available.

RAD50 (S. cerevisiae; homolog of; RAD50): In yeast this gene aids in the repair of double stranded DNA breaks by end repair and chromosomal integration (ALT pathway of telomere maintenance). It is thought to have much the same function in humans as it has been found to associate with the TRF2 complex previously described.

RAD51 (S. cerevisiae; homolog of; RAD51): In prokaryotic cells this gene encodes for proteins responsible for promoting strand exchange between homologous sections of double stranded DNA (this is also part of the ALT pathway of telomere maintenance). In eukaryotic cells, the function is also thought to be similar and involved in replication and strand exchange.

RAP1 (GTPase Activating Protein 1): Encodes for a member of a family of p21 proteins whose activities are related to binding and hydrolysis of GTP and function in cell differentiation and growth.

Reactive Oxygen Species (ROS): Very small, organic or inorganic, highly reactive ions or molecules having unpaired electrons in a valence shell including but not limited to free radicals, peroxides and oxygen ions.

Recloning: The process in which a genetically identical organism is made from the genetic material of a previously cloned (creating genetically identical organisms from the genetic material of a single “parent” organism through the use of genetic recombination and in vitro fertilization) organism.

Resveratrol (3,5,4′-trihydroxystilbene) belongs to a family of compounds known as phytoalexins. These compounds are synthesized by various plants including grapes, knotweed, blueberries, some pine trees and other plants as part of their natural defense mechanisms in response to stress, injury, invasion by fungi or UV damage. In grapes they are concentrated in the grape skin where they protect from UV damage and function as anti bacterial and anti viral agents. Resveratrol activates the sirtuins which are enzymes which produce at least part of the effects of caloric restriction in living organisms and caloric restriction has been shown in a very wide range of species tested to extend the lifespan of those organisms.

The activation of a sirtuin deacetylase protein family member may be used to produce lifespan extension by mimicking caloric restriction in contrast to extending lifespan by protecting or repairing telomeric structure in cells. Activating compounds may be polyphenol(s) from plants such as chalcone, stilbene, flavone or other sirtuin modulating compounds derived from plants or created by other synthetic processes described herein. Methods for the isolation and characterization of resveratrol are well known in the art; in addition, this compound is commercially available.

Ribosome: A structure of protein and RNA involved in translation, or the expression of genetic code from nucleic acid into protein.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of nucleic acid or amino acid sequences will possess a relatively high degree of sequence identity when aligned using standard methods. This homology will be more significant when the orthologous proteins or nucleic acids are derived from species which are more closely related (e.g., human and chimpanzee sequences), compared to species more distantly related (e.g., human and C. elegans sequences). Typically, orthologs are at least 50% identical at the nucleotide level and at least 50% identical at the amino acid level when comparing human orthologous sequences.

Methods of alignment of sequences for comparison are well known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. Biosci. 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. Each of these sources also provides a description of how to determine sequence identity using this program.

Homologous sequences are typically characterized by possession of at least 60%, 70%, 75%, 80%, 90%, 95% or at least 98% sequence identity counted over the full length alignment with a sequence using the NCBI Blast 2.0, gapped blastp set to default parameters. Queries searched with the blastn program are filtered with DUST (Hancock and Armstrong, Comput. Appl. Biosci. 10:67-70, 1994). It will be appreciated that these sequence identity ranges are provided for guidance only; it is entirely possible that strongly significant homologs could be obtained that fall outside of the ranges provided.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that all encode substantially the same protein.

An alternative indication that two nucleic acid molecules are closely related is that the two molecules hybridize to each other under stringent conditions, as described under “specific hybridization.”

SHC1 (SHC Transforming Protein 1): By coupling growth factor receptors to members of the RAS pathway, mitogenic signal transduction is regulated. The protein encoded by SHC1 acts as an adaptor in many signaling pathways specifically translating reactive oxygen damage into cell death.

Small interfering RNAs (siRNAs): Synthetic or naturally-produced small double stranded RNAs (dsRNAs) that can induce gene-specific inhibition of expression in invertebrate and vertebrate species are provided. These RNAs are suitable for interference or inhibition of expression of a target gene and comprise double stranded RNAs of about 15 to about 40 nucleotides (for instance, 20-25 nucleotides), often containing a 3′ and/or 5′ overhang on each strand having a length of 0- to about 5-nucleotides, wherein the sequence of the double stranded RNAs is substantially identical to a portion of a coding region of the target gene for which interference or inhibition of expression is desired. The double stranded RNAs can be formed from complementary ssRNAs or from a single stranded RNA that forms a hairpin or from expression from a DNA vector. These molecules function in RNA silencing a method in which sequence specific gene expression is reduced/eliminated by the incorporation of the siRNA in to the RNA induced silencing complex that facilitates the degradation of the targeted mRNA.

Specific hybridization: Specific hybridization refers to the binding, duplexing, or hybridizing of a molecule only or substantially only to a particular nucleotide sequence when that sequence is present in a complex mixture (e.g. total cellular DNA or RNA). Specific hybridization may also occur under conditions of varying stringency.

Hybridization conditions resulting in particular degrees of stringency will vary depending upon the nature of the hybridization method of choice and the composition and length of the hybridizing DNA used. Generally, the temperature of hybridization and the ionic strength (especially the Na+ concentration) of the hybridization buffer will determine the stringency of hybridization. Calculations regarding hybridization conditions required for attaining particular degrees of stringency are discussed by Sambrook et al. (In: Molecular Cloning: A Laboratory Manual, Cold Spring Harbor, N.Y., 1989 ch. 9 and 11). By way of illustration only, a hybridization experiment may be performed by hybridization of a DNA molecule to a target DNA molecule which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989).

Traditional hybridization with a target nucleic acid molecule labeled with [³²P]-dCTP is generally carried out in a solution of high ionic strength such as 6×SSC at a temperature that is 20-25° C. below the melting temperature, Tₘ, described below. For Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal.

The term Tₘ represents the temperature (under defined ionic strength, pH and nucleic acid concentration) at which 50% of the probes complementary to the target sequence hybridize to the target sequence at equilibrium. Because the target sequences are generally present in excess, at Tₘ 50% of the probes are occupied at equilibrium. The Tₘ of such a hybrid molecule may be estimated from the following equation (Bolton and McCarthy, Proc. Natl. Acad. Sci. USA 48:1390, 1962):

Tₘ=81.5° C.−16.6(log₁₀Na⁺)+0.41(% G+C)−0.63(% formamide)−(600/1)

where 1=the length of the hybrid in base pairs.

This equation is valid for concentrations of Na⁺ in the range of 0.01 M to 0.4 M, and it is less accurate for calculations of Tm in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al. (Molecular Cloning: A Laboratory Manual, Cold Spring Harbor, N.Y., 1989).

Thus, by way of example, for a 150 base pair DNA probe derived from a cDNA (with a hypothetical % GC of 45%), a calculation of hybridization conditions required to give particular stringencies may be made as follows: For this example, it is assumed that the filter will be washed in 0.3×SSC solution following hybridization, thereby: [Na⁺]=0.045 M; % GC=45%; Formamide concentration=0; 1=150 base pairs; Tm=81.5−16.6(log₁₀ [Na⁺])+(0.41×45)−(600/150); and so Tₘ=74.4° C.

The Tₘ of double-stranded DNA decreases by 1-1.5° C. with every 1% decrease in homology (Bonner et al., J. Mol. Biol. 81:123, 1973). Therefore, for this given example, washing the filter in 0.3×SSC at 59.4-64.4° C. will produce a stringency of hybridization equivalent to 90%; that is, DNA molecules with more than 10% sequence variation relative to the target cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target cDNA molecule will not hybridize. The above example is given entirely by way of theoretical illustration. It will be appreciated that other hybridization techniques may be utilized and that variations in experimental conditions will necessitate alternative calculations for stringency.

Stringent conditions may be defined as those under which DNA molecules with more than 25%, 15%, 10%, 6% or 2% sequence variation (also termed “mismatch”) will not hybridize. Stringent conditions are sequence dependent and are different in different circumstances. Longer sequences hybridize specifically at higher temperatures. Generally, stringent conditions are selected to be about 5° C. lower than the thermal melting point Tₘ for the specific sequence at a defined ionic strength and pH. An example of stringent conditions is a salt concentration of at least about 0.01 to 1.0 M Na ion concentration (or other salts) at pH 7.0 to 8.3 and a temperature of at least about 30° C. for short probes (e.g. 10 to 50 nucleotides). Stringent conditions can also be achieved with the addition of destabilizing agents such as formamide. For example, conditions of 5×SSPE (750 mM NaCl, 50 mM Na phosphate, 5 mM EDTA, pH 7.4) and a temperature of 25-30° C. are suitable for allele-specific probe hybridizations.

A perfectly matched probe has a sequence perfectly complementary to a particular target sequence. The test probe is typically perfectly complementary to a portion (subsequence) of the target sequence. The term “mismatch probe” refers to probes whose sequence is deliberately selected not to be perfectly complementary to a particular target sequence.

Transcription levels can be quantitated absolutely or relatively. Absolute quantitation can be accomplished by inclusion of known concentrations of one or more target nucleic acids (for example control nucleic acids or with a known amount the target nucleic acids themselves) and referencing the hybridization intensity of unknowns with the known target nucleic acids (for example by generation of a standard curve).

Solid support (or substrate): Any material which is insoluble, or can be made insoluble by a subsequent reaction. Numerous and varied solid supports are known to those in the art and include, without limitation, nitrocellulose, the walls of wells of a reaction tray, test tubes, polystyrene beads, magnetic beads, membranes, microparticles (such as latex particles), and sheep (or other animal) red blood cells. Any suitable porous material with sufficient porosity to allow access by detector reagents and a suitable surface affinity to immobilize capture reagents (e.g., monoclonal antibodies) is contemplated by this term. For example, the porous structure of nitrocellulose has excellent absorption and adsorption qualities for a wide variety of reagents, for instance, capture reagents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

It is contemplated that porous solid supports, such as nitrocellulose, described hereinabove are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

The surface of a solid support may be activated by chemical processes that cause covalent linkage of an agent (e.g., a capture reagent) to the support. However, any other suitable method may be used for immobilizing an agent (e.g., a capture reagent) to a solid support including, without limitation, ionic interactions, hydrophobic interactions, covalent interactions and the like. The particular forces that result in immobilization of an agent on a solid phase are not important for the methods and devices described herein.

A solid phase can be chosen for its intrinsic ability to attract and immobilize an agent, such as a capture reagent. Alternatively, the solid phase can possess a factor that has the ability to attract and immobilize an agent, such as a capture reagent. The factor can include a charged substance that is oppositely charged with respect to, for example, the capture reagent itself or to a charged substance conjugated to the capture reagent. In another embodiment, a specific binding member may be immobilized upon the solid phase to immobilize its binding partner (e.g., a capture reagent). In this example, therefore, the specific binding member enables the indirect binding of the capture reagent to a solid phase material.

Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

Stressed Cells: Cells not able to function fully in their expected capacity either through chemical, biological, or mechanical interference by an outside agent including but not limited to: free radicals, ROS, Toxins, UV radiation and genetic inhibitors like siRNAs.

Suffruticosol A and B: Stilbenes (a hydrocarbon with a trans ethane double bond substituted with a phenyl group on both carbon atoms of the double bond), resveratrol derivatives from peony seeds having antioxidant properties and mimicking the effects of resveratrol.

Tea: An aqueous extract of plant material, usually temperature modulated (hot or cold); often the extracted material is leaves (commonly, but not limited to dried and/or fermented leaves of Camellia sinensis green or black tea; including white tea), though teas can be made from other plant material including bark, flowers, seeds, seed hulls, and so forth. EGCG a primary element of the tea extract (as well as all ester-bond containing polyphenols) has been able to show a pronounced inhibitory effect on certain types of cancer cells thought to be through a proteosome inhibition mechanism, while the non ester bond containing polyphenols have shown diminished or no such effect. In the case of lifespan extension through telomere length maintenance mechanisms no such distinction is made with the belief that all forms of polyphenols have an effect.

Telomerase: The enzyme (DNA polymerase) primarily responsible for repairing damage to the special chromatin structures at the end of chromosomes known as telomeres. Telomerase adds specific DNA sequence repeats (TTAGGG in all vertebrates) to the 3′ end of DNA strands in telomeres, which are found at the ends of eukaryotic chromosomes. Telomerase functions as a reverse transcriptase, and is associated with a RNA molecule that acts as a template for elongating telomeres that have been shortened after replication.

Telomere Unit: The telomere (repetitive sequence at the end of most eukaryotic chromosomes composed of chromatin) and all associated proteins, enzymes and genetic sequences including, but not limited to: TERT, TRF1, TERF2, TERF21P, DNA Polymerase, POLG, POLB, POLD3, POLE, POLI, POLL, PARP1, PARP2, PPARG, SHC1, HSPA1A, HSPA1B, and HSPA1L.

TERC (Telomerase RNA Component): A human gene that serves as the template for the telomeric repeat known as the telomerase RNA component.

TERF2 (Telomeric Repeat Binding Factor 2): Plays a vital role in the protective activity of telomeres. TERF2 may convert the telomeres into large duplex loops (called t loops) that may provide a general mechanism for the protection and replication of telomeres.

TERF2IP (TERF2 Interacting Protein): A ubiquitously expressed protein recruited to telomeres by TERF2 to regulate telomere length. This protein is involved in activation of RNA polymerase II and central to cellular function and efficiency during rapid growth events.

TFAM (Transcription Factor A; mitochondrial): Activates mitochondrial transcription by binding to nucleotides present in both light and heavy promoters. Also plays a promoter role in the mitochondrial replication process through formation of an RNA primer.

TIN2 (TRF1 Interacting Nuclear Factor 2): Telomere length in humans is partly controlled by a feedback mechanism in which telomere elongation by telomerase is limited by the accumulation of the TRF1 complex at chromosome ends. TRF1 itself can be inhibited by PARP activity of its interacting partner tankyrase-1 which disables binding capabilities and removes TRF1 complex from telomeres. TIN2 is ma mediator of tankyrase 1 and the TRF1 complex. Transient inhibition of TIN2 with small interfering RNA led to diminished telomeric TRF1 signals. These and other data identified TIN2 as a PARP modulator in the TRF1 complex and explained how TIN2 contributes to the regulation of telomere length.

Transcription: The process by which DNA directs the synthesis of RNA by serving as the nucleotide sequence template for the formation of the RNA nucleotide sequence.

TPP1 (Tripeptidyl Peptidase 1): Lysosomal protein that catalyzes the removal of an amino acid from a polypeptide chain, specifically it sequentially removes tripeptides from the N termini of proteins.

TRF1 (Telomeric Repeat Binding Factor 1): Regulates telomere length via binding to the TTAGGG sites and tankyrase, TIN2 and PINX1. The TRF1 complex interacts with POT1 (protection of telomeres-1; a single stranded telomeric binding protein) which controls telomerase mediated telomere elongation.

Ubiquinone (also known as Coenzyme Q10): A key component of the electron transport/cellular respiration/energy production mechanism, ubiquinone is found in the mitochondria of most eukaryotic cells and in great abundance in cells that have high energy requirements (heart, liver, etc.). Through the process of aerobic cellular respiration ATP is created for use by the cell (95% of all energy in the human body is created in this fashion). Ubiquinone has an affinity for electron transfer and is intimately involved in mitochondrial cellular respiration specifically between Complex II and III where it acts as a transfer agent. Since ubiquinone is a Redox (oxidative reduction) agent, it demonstrates free radical quenching capabilities. The fully oxidized form of the compound is known as ubiquinone, when absorbed into the body 90% of it converts to the “active” antioxidant form of ubiquinol. Methods for the isolation and characterization of ubiquinone are well known in the art; in addition, this compound is commercially available.

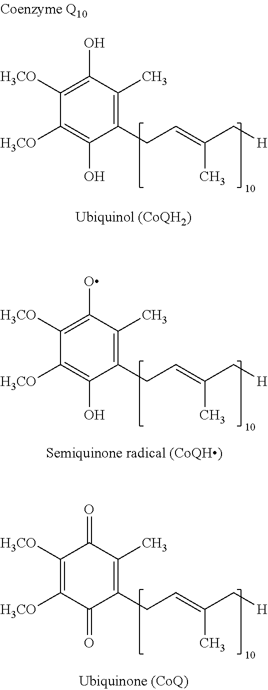

UVA1: A subset of wavelengths in one of the three “bands” of solar lights Ultraviolet Radiation (UVA, UVB and UVC) in the relatively higher power, longer wavelength range of 340 nm-400 nm. UVA2: Solar radiation wavelength range of 320 nm-340 nm. UVB: Solar radiation between the wavelengths of 280 nm-315 nm, capable of causing direct damage to the DNA of cells. UVC: The short, highest energy wavelength radiation (100 nm-280 nm) that is generally filtered by the atmosphere.

Viniferin: A stilbene (a hydrocarbon with a trans ethane double bond substituted with a phenyl group on both carbon atoms of the double bond), resveratrol derivative from peony seeds having antioxidant properties and mimicking the effects of resveratrol.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Provided herein in a first embodiment is a method for modulating the lifespan of a cell, tissue, organ or organism, comprising contacting the cell, tissue, organ or organism with one (or more) of the compounds or compositions discussed herein, such as idebenone, or an analog or derivative thereof; a cocoa extract; a coffee cherry extract; quinic acid, or an analog or derivative thereof; ferulic acid, or an analog or derivative thereof; a proanthocyanidin, anthocyanidin, procyanidin, or cyanidin; chlorogenic acid, or an analog or derivative thereof; a tea extract; or resveratrol or a composition derived from or chemically related to resveratrol. By way of example, the coffee cherry extract in some instances comprises one or more of chlorogenic acid, quinic acid, ferulic acid, caffeic acid or proanthocyanidins. In another example, tea extract comprises one or more polyphenols selected from EGCG (epigallocatechin-3-gallate), EGC (epigallocatechin), ECG (epicatechin-3-gallate), EC (epicatechin), GCG (gallocatechin gallate), GC (gallocatechin), C (catechin) and CG (catechin gallate). In yet another example, the composition derived from or chemically related to resveratrol is selected from the group consisting of viniferin, gnetin H, and suffruticosol B. In another example, the cocoa extract comprises a polyphenol and/or procyanidin selected from (+) catechin, (−) epicatechin, procyanidin oligomers 2 through 18, procyanidin B-5, procyanidin B-2, procyanidin A-2 and/or procyanidin C-1.

In various of the provided embodiments, modulating the lifespan comprises modulating the level and/or activity of at least one gene selected from the group consisting of those listed in Data Table 7 and those listed as part of Array 2. For instance, modulating comprises (in some cases) increasing the level of activity of the at least one listed gene. In other cases, there is provided a method wherein modulating comprises decreasing the level of activity of the at least one listed gene.

Also provided are methods for modulating the lifespan of a cell, tissue, organ or organism, wherein modulating comprises modulating the level and/or activity of: ten or more of the genes listed as part of Array 2; the genes listed as part of Array 1; VEGFA, HMOX1, CCL4L1, DDC, NOS2A, SIRT1, TERT, PTGS2, or IFI44; four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70; BCL2, SOD1, TP53, and SOD2; BCL2, SOD1, TP53, SOD2, BCL2L1, TIMM22, TOMM40, IMMP1L, CDKN2A, GADPH, ACTB, HRP1, and HGDC; PARP1, PARP2, TERT, TEP1, TPS3, JUN, PARP3, PARP4, TERF2, TINF2, and CDKN2A; PARP1, PARP2, TERT, TEP1, and TP53; TERF2, POT1, TERT, and TPP1; PAPR1, PARP2, PARP3, and PARP4; PARP2, CYP19A1, TEP1, BCL2, HSPA1A, ACE, TP53, and NFKB1; IGF1, IGF2, PPARG, IL10, APOE, TERT, TNF, HLA-DRA, DDC, CCL4L1, NOS2A, and GH1; PARP1, IL6, SIRTT1, KRAS, and HSPA1L; IGF1, IL6, PPARG, IL10, TERT, TNF, TEP1, HSPA1A, SIRT1, TP53, GH1, NOS2A, and PPC; or another list of genes described herein; or a combination of two or more of these specific lists.

In one embodiment, modulating the lifespan comprises modulating the activity or level of at least one of the telomere length maintenance genes, for instance increasing the level or activity of at least one telomere length maintenance gene or decreasing the level or activity of at least one telomere length maintenance gene.

In another embodiment, modulating the lifespan comprises modulating the activity or level of f telomerase, for instance increasing the level or activity of telomerase or decreasing the level or activity of telomerase. Also provided are methods that involve differentially modulating the activity of one or more telomere length maintenance genes so that the lifespan of healthy cells is increased and/or the lifespan of unhealthy, diseased, damaged or cancerous cells is decreased.

In any of the provided methods, the method can take place in a cell that is in vitro. Optionally, the cell is a mammalian cell (e.g., cells are selected from keratinocytes, fibroblasts, melanocytes, endothelial cells, langerhans cells, merkel cells, adipocytes, nerve cells, hair, sweat, oil, stem cells and/or muscle cells), a plant cell, a microbial cell, a stem cell, an autologous cell or an allograft cell, an embryo or in vitro fertilization cell. In different embodiments, the cell is a eukaryotic cell or a a prokaryotic cell.

Provided in yet another embodiment is a method for modulating response or resistance to stress of a cell, tissue, organ or organism, comprising modulating the level and/or activity of at least one gene selected from the group consisting of those listed in Data Table 7 and those listed as part of Array 2. Also provided are methods for modulating response or resistance to stress, wherein modulating comprises modulating the level and/or activity of: ten or more of the genes listed as part of Array 2; the genes listed as part of Array 1; VEGFA, HMOX1, CCL4L1, DDC, NOS2A, SIRT1, TERT, PTGS2, or IFI44; four or more of TERT, TERC, NRF2, POT1, TRF1, TRF2, TIN2, TPP1, RAPT, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70; BCL2, SOD1, TP53, and SOD2; BCL2, SOD1, TP53, SOD2, BCL2L1, TIMM22, TOMM40, IMMP1L, CDKN2A, GADPH, ACTB, HRP1, and HGDC; PARP1, PARP2, TERT, TEP1, TPS3, JUN, PARP3, PARP4, TERF2, TINF2, and CDKN2A; PARP1, PARP2, TERT, TEP1, and TP53; TERF2, POT1, TERT, and TPP1; PAPR1, PARP2, PARP3, and PARP4; PARP2, CYP19A1, TEP1, BCL2, HSPA1A, ACE, TP53, and NFKB1; IGF1, IGF2, PPARG, IL10, APOE, TERT, TNF, HLA-DRA, DDC, CCL4L1, NOS2A, and GH1; PARP1, IL6, SIRTT1, KRAS, and HSPA1L; IGF1, IL6, PPARG, IL10, TERT, TNF, TEP1, HSPA1A, SIRT1, TP53, GH1, NOS2A, and PPC; or another list of genes described herein; or a combination of two or more of these specific lists. By way of example, modulating in such methods may involve increasing the level of activity of the at least one listed gene, or decreasing the level of activity of the at least one listed gene, or increasing some while decreasing others.

Yet another embodiment is a method of increasing or decreasing cellular respiration and/or capacity and/or biogenesis of mitochondria in a cell, by contacting the cell with at least one lifespan modulating agent discussed herein, such as idebenone, or an analog or derivative thereof; a cocoa extract; a coffee cherry extract; quinic acid, or an analog or derivative thereof; ferulic acid, or an analog or derivative thereof; a proanthocyanidin, anthocyanidin, procyanidin, or cyanidin; chlorogenic acid, or an analog or derivative thereof; a tea extract; or resveratrol or a composition derived from or chemically related to resveratrol. By way of example, the coffee cherry extract in some instances comprises one or more of chlorogenic acid, quinic acid, ferulic acid, caffeic acid or proanthocyanidins. In another example, tea extract comprises one or more polyphenols selected from EGCG (epigallocatechin-3-gallate), EGC (epigallocatechin), ECG (epicatechin-3-gallate), EC (epicatechin), GCG (gallocatechin gallate), GC (gallocatechin), C (catechin) and CG (catechin gallate). In yet another example, the composition derived from or chemically related to resveratrol is selected from the group consisting of viniferin, gnetin H, and suffruticosol B. In another example, the cocoa extract comprises a polyphenol and/or procyanidin selected from (+) catechin, (−) epicatechin, procyanidin oligomers 2 through 18, procyanidin B-5, procyanidin B-2, procyanidin A-2 and/or procyanidin C-1.

In another example of such methods, the method comprises increasing the lifespan of a cell through modulating biogenesis of, or respiratory efficiency of mitochondria, lengthening telomeres, and/or modulating at least one gene affecting the same.

Also provided are such methods, comprising increasing or decreasing proliferation or biogenesis of mitochondria through modulation of at least one of PGC 1α, SIRT1, SIRT3, SIRT4, SIRT5, NRF1 and/or Tfam.

Any of the provided methods optionally also includes inducing mitochondrial regeneration, or new mitochondrial biosynthesis in at least one cell.

Yet another embodiment is a method for modulating, preventing, delaying, or reversing acute cell death or apoptosis, or prolonging the survival of a cell, tissue, organ or organism comprising modulating the level and/or activity of at least one gene selected from the group consisting of those listed in Data Table 7 and those listed as part of Array 2. For instance, modulating acute cell death or apoptosis comprises increasing or upregulating acute cell death or apoptosis.

Another provided method is for modulating, enhancing, maintaining or producing a more youthful or function of the skin and/or associated tissues, comprising modulating the level and/or activity of at least one gene selected from the group consisting of those listed in Data Table 7 and those listed as part of Array 2.

The methods provided herein may involve modulating the level or activity of the at least one gene comprising contacting a cell with an antisense or siRNA molecule.

Also provided are collections of lifespan-influencing nucleic acid molecules, which collection comprises a plurality of nucleic acid molecules selected from those listed in Data Table 7 or Array 2, or fragments of those listed in Data Table 7 or Array 2. Optionally, such collections are affixed to solid surface in an array such as for instance a microarray.

Also provided are methods of screening compounds useful for modulating lifespan, the methods involving contacting a test compound with a host cell expresses a lifespan-influencing protein encoded by an isolated nucleic acid molecule listed in Data Table 7 or listed as part of Array 2 and detecting a change in the expression of the nucleotide sequence or a change in activity of encoded protein, wherein such a change indicates the test compound is useful for modulating lifespan. Optionally, such methods are high throughput methods (ror instance, in an array format), involving contacting in parallel a test compound with a collection of host cells each of which expresses a different lifespan-influencing protein encoded by an isolated nucleic acid molecule in listed in Data Table 7 or listed as part of Array 2; and detecting a change in the expression of at least one of the nucleotide sequences or a change in activity of at least one of the encoding proteins, wherein such a change indicates the test compound(s) are useful for modulating lifespan.

Another method described herein is a method for identifying an agent with potential to reverse or inhibit mitochondrial damage, comprising: contacting an cell with an agent; and detecting the level of a nucleic acid molecule corresponding to ACTB, BCL2, BCL2L1, CDKN2A, COX10, COX18, CPT1B, CPT2, DNAJC19, EGF, EGR2, FIST, GAPDH, GRPEL1, HSP90AA1, LRPPRC, MFN1, MFN2, NOS3, OPA1, PARP3, PARP4, PPARGC1A, SIRT2, SIRT4, SLC25A1, SLC25A1, SLC24A2, SLC25A3, SLC25A4, SCL25A5, SLC25A10, SLC25A12, SLC25A13, SLC25A14, SLC25A15, SLC25A16, SLC25A17, SLC25A19, SLC25A2, SLC25A20, SLC25A21, SLC25A22, SLC25A_{—}23, SLC25A24, SLC25A25, SLC25A27, SLC25A3, SLC25A30, SLC25A31, SLC25A37, SLC25A4, SLC25A5, TIMM10, TIMM17A, TIMM17B, TIMM22, TIMM23, TIMM44, TIMM50, TIMM8A, TIMM8B, TIMM9, TOMM20, TOMM22, TOMM34, TOMM40, TOMM40L, TOMM70A, UCP1, UCP2, UCP3 or another gene indicated herein as beneficial for mitochondrial health or maintenance when increased, or the level or activity of a protein encoded thereby, in the presence and absence of the agent, wherein an increase in the level or activity in the presence of the agent as compared to in the absence of the agent indicates that the agent has potential to reverse or inhibit mitochondrial damage.

Yet another method described herein is a method for identifying an agent with potential to reverse or inhibit mitochondrial damage, comprising: contacting an cell with an agent; and detecting the level of a nucleic acid molecule corresponding to AIFM2, AIP, BAK1, BBC3, BID, BNIP3, CLK1, HSPA1A, HSPA1B, HSPA1L, IMMP1L, IMMP2L, MIPEP, PARP1, PARP2, PMAIP1, RPL13A, SOD1, SOD2, SFN, SH3GLB1, UXT or another gene indicated herein as beneficial for mitochondrial health or maintenance when decreased, or the level or activity of a protein encoded thereby, in the presence and absence of the agent, wherein a decrease in the level or activity in the presence of the agent as compared to in the absence of the agent indicates that the agent has potential to reverse or inhibit mitochondrial damage.

Still another method described herein is a method for identifying an agent with potential to increase or accelerate mitochondrial damage, comprising: contacting an cell with an agent; and detecting the level of a nucleic acid molecule corresponding to ACTB, BCL2, BCL2L1, CDKN2A, COX10, COX18, CPT1B, CPT2, DNAJC19, EGF, EGR2, FIS1, GAPDH, GRPEL1, HSP90AA1, LRPPRC, MFN1, MFN2, NOS3, OPA1, PARP3, PARP4, PPARGC1A, SIRT2, SIRT4, SLC25A1, SLC25A1, SLC24A2, SLC25A3, SLC25A4, SCL25A5, SLC25A10, SLC25A12, SLC25A13, SLC25A14, SLC25A15, SLC25A16, SLC25A17, SLC25A19, SLC25A2, SLC25A20, SLC25A21, SLC25A22, SLC25A_{—}23, SLC25A24, SLC25A25, SLC25A27, SLC25A3, SLC25A30, SLC25A31, SLC25A37, SLC25A4, SLC25A5, TIMM10, TIMM17A, TIMM17B, TIMM22, TIMM23, TIMM44, TIMM50, TIMM8A, TIMM8B, TIMM9, TOMM20, TOMM22, TOMM34, TOMM40, TOMM40L, TOMM70A, UCP1, UCP2, UCP3 or another gene indicated herein as beneficial for mitochondrial health or maintenance when increased, or the level or activity of a protein encoded thereby, in the presence and absence of the agent, wherein a decrease in the level or activity in the presence of the agent as compared to in the absence of the agent indicates that the agent has potential to increase or accelerate mitochondrial damage.

Another method described herein is a method for identifying an agent with potential to increase or accelerate mitochondrial damage, comprising: contacting an cell with an agent; and detecting the level of a nucleic acid molecule corresponding to AIFM2, AIP, BAK1, BBC3, BID, BNIP3, CLK1, HSPA1A, HSPA1B, HSPA1L, IMMP1L, IMMP2L, MIPEP, PARP1, PARP2, PMAIP1, RPL13A, SOD1, SOD2, SFN, SH3GLB 1, UXT or another gene indicated herein as beneficial for mitochondrial health or maintenance when decreased, or the level or activity of a protein encoded thereby, in the presence and absence of the agent, wherein an increase in the level or activity in the presence of the agent as compared to in the absence of the agent indicates that the agent has potential to increase or accelerate mitochondrial damage.

In another embodiment, there is provided a method for identifying an agent with potential to reverse or inhibit DNA damage or telomere shortening, comprising: contacting an cell with an agent; and detecting the level of a nucleic acid molecule corresponding to AK3, APEX1, APEX2, ATF2, ATM, ATR, ATRX, BARD1, BLM, BRIP1, CCNH, CDK7, CDKN2A, CHEK1, CHEK2, CSF2, CTPS, DDB1, DDB2, DHFR, DMC1, ERCC1, ERCC2, ERCC3, ERCC4, ERCC5, ERCC6, ERCC8, EXO1, FANCA, FANCC, FANCF, FANCG, FEN1, GADD45A, GADD45G, GTF2H1, GTF2H2, GTF2H3, GTF2H4, JUN, LIG1, LIG3, LIG4, MAP2K6, MAPKAPK2, MLH1, MLH3, MRE11A, MSH2, MSH3, MSH4, MSH5, MSH6, NBN, NEIL1, NEIL2, NEIL3, NFKB1, NFKBIA, HK1, NUDT1, NUDT2, ODC1, PAPSS1, PAPSS2, PARP1, PARP3, PCNA, PMS1, PMS2, PNKP, POLB, POLD3, POLE, POLI, POLL, PRKDC, RAD1, RAD18, RAD21, RAD23A, RAD50, RAD51C, RAD51L1, RAD51L3, RAD52, RAD54B, RAD54L, RBBP8, SESN1, SLC23A2, TDG, TYMS, UBE2V2, UNG2, WRN, XAB2, XPA, XPC, XRCC1, XRCC2, XRCC3, XRCC4, XRCC5, XRCC6, ZNRD1 or another gene indicated herein as beneficial for DNA or telomere maintenance when increased, or the level or activity of a protein encoded thereby, in the presence and absence of the agent, wherein an increase in the level or activity in the presence of the agent as compared to in the absence of the agent indicates that the agent has potential to reverse or inhibit DNA damage or telomere shortening.

Another embodiment is a method for identifying an agent with potential to reverse or inhibit DNA damage or telomere shortening, comprising: contacting an cell with an agent; and detecting the level of a nucleic acid molecule corresponding to B2M, BRCA1, BRCA2, BTG2, CIDEA, CIDEB, DDIT3, DKC1, GTSE1, MDM2, PCBP4, PDCD8, PINX1, PPP1R15A, RAD17, RELA, TELO2, TEP1 or another gene indicated herein as beneficial for DNA or telomere maintenance when decreased, or the level or activity of a protein encoded thereby, in the presence and absence of the agent, wherein a decrease in the level or activity in the presence of the agent as compared to in the absence of the agent indicates that the agent has potential to reverse or inhibit DNA damage or telomere shortening.

Also provided is a method for identifying an agent with potential to accelerate or cause or enhance DNA damage or telomere shortening, comprising: contacting an cell with an agent; and detecting the level of a nucleic acid molecule corresponding to AK3, APEX1, APEX2, ATF2, ATM, ATR, ATRX, BARD1, BLM, BRIP1, CCNH, CDK7, CDKN2A, CHEK1, CHEK2, CSF2, CTPS, DDB1, DDB2, DHFR, DMC1, ERCC1, ERCC2, ERCC3, ERCC4, ERCC5, ERCC6, ERCC8, EXO1, FANCA, FANCC, FANCF, FANCG, FEN1, GADD45A, GADD45G, GTF2H1, GTF2H2, GTF2H3, GTF2H4, JUN, LIG1, LIG3, LIG4, MAP2K6, MAPKAPK2, MLH1, MLH3, MRE11A, MSH2, MSH3, MSH4, MSH5, MSH6, NBN, NEIL1, NEIL2, NEIL3, NFKB1, NFKBIA, HK1, NUDT1, NUDT2, ODC1, PAPSS1, PAPSS2, PARP1, PARP3, PCNA, PMS1, PMS2, PNKP, POLB, POLD3, POLE, POLI, POLL, PRKDC, RAD1, RAD18, RAD21, RAD23A, RAD50, RAD51C, RAD51L1, RAD51L3, RAD52, RAD54B, RAD54L, RBBP8, SESN1, SLC23A2, TDG, TYMS, UBE2V2, UNG2, WRN, XAB2, XPA, XPC, XRCC1, XRCC2, XRCC3, XRCC4, XRCC5, XRCC6, ZNRD1 or another gene indicated herein as beneficial for DNA or telomere maintenance when increased, or the level or activity of a protein encoded thereby, in the presence and absence of the agent, wherein a decrease in the level or activity in the presence of the agent as compared to in the absence of the agent indicates that the agent has potential to accelerate or cause or enhance DNA damage or telomere shortening.

Another provided method is a method for identifying an agent with potential to accelerate or cause or enhance DNA damage or telomere shortening, comprising: contacting an cell with an agent; and detecting the level of a nucleic acid molecule corresponding to B2M, BRCA1, BRCA2, BTG2, CIDEA, CIDEB, DDIT3, DKC1, GTSE1, MDM2, PCBP4, PDCD8, PINX1, PPP1R15A, RAD17, RELA, TELO2, TEP1 or another gene indicated herein as beneficial for DNA or telomere maintenance when decreased, or the level or activity of a protein encoded thereby, in the presence and absence of the agent, wherein an increase in the level or activity in the presence of the agent as compared to in the absence of the agent indicates that the agent has potential to accelerate or cause or enhance DNA damage or telomere shortening.

Further embodiments exploit the discovery made herein that the dosage of compounds applied has a profound effect on the up and/or down regulation of various genes. Thus, there is provided a first method for inducing expression of TERT, POT1, TPP1 and TERF2 in a cell, by applying to the cell or an organism comprising the cell a composition comprising between about 0.000001% and about 10% (by weight) coffee cherry extract. In certain embodiments of this method the composition comprises no more than about 0.01% (by weight) coffee cherry extract. Alternatively, the composition further comprises green tea extract, a component of green tea extract, or idebenone, such as for instance one or more of about 0.001% (by weight) green tea extract or about 0.00004% (by weight) idebenone.

Also provided is a method inducing expression of PARP1, BCL2 and p53 in a cell, by applying to the cell or an organism comprising the cell a composition comprising between about 0.000001% and about 10% (by weight) coffee cherry extract. In certain examples of this method, the composition comprises no more than about 0.000005% (by weight) chlorogenic acid.

Yet another embodiment is a method of inducing expression of NOS2A, NOS1, and NOS3 in a cell, by applying to the cell or an organism comprising the cell a composition comprising between about 0.000001% and about 10% (by weight) coffee cherry extract, or wherein the composition comprises no more than about 0.01% coffee cherry extract.

In another embodiment, there is provide method of inducing expression of CCL4L1 in a cell, by applying to the cell or an organism comprising the cell a composition comprising between about 0.000001% and about 10% (by weight) coffee cherry extract. Examples of this method involve using a composition that comprises no more than about 0.01% (by weight) coffee cherry extract.

Still other embodiments are described herein, and this list is not intended to be exhaustive.

### IV. Lifespan Extension

Many factors have been shown to diminish lifespan in living creatures, but few have clearly demonstrated extension of lifespan.

One example of lifespan extension however, is a dietary program termed caloric restriction which is well documented to extend the lifespan in a variety of living organisms. Agents which can mimic some of the effects of caloric restriction or increase a cell's resistance to stress have been described with the NAD+salvage pathway. Members of a family of genes termed Silent Information Regulators (SIR) are involved in various processes of gene silencing and DNA repair. It has been shown that yeasts which are lacking the SIR2 gene do not live longer when calorically restricted and thus it is believed that the SIR2 gene mediates some of the beneficial lifespan extending effects of caloric restriction. Other genes in this family include SIRT1 which may have a similar effect.

Sirtuin modulating compounds then have use in extending the lifespan of certain living cells as well as having the potential to treat and/or prevent various diseases related to aging. One such example is resveratrol which is a naturally occurring substance in red wine which has been shown to increase lifespan in mice and which appears to affect in some genes of the sirtuin family (although it also alters the gene expression and/or protein production by many other genes).

Relatively minor changes at a genetic level have been shown to significantly alter the aging process as have various environmental factors. The rate of aging, the health of the organism as the aging process progresses as well as the total lifespan are complexly controlled and are the subject of various theories of aging.

### V. Modification of Lifespan

Provided herein are the results of comprehensive analyses of gene expression changes in the presence of antioxidant compounds, with and without pre-stimulation with a stressor (e.g., an environmental stress such as ultraviolet radiation exposure). Also provided are dosage response analyses, illustrating the changes in gene responses with changes in the amount of antioxidant compounds. Based on the results provided herein, methods are now enabled for affecting such expression changes in order to influence (increase or decrease) the health or lifespan of cells, tissues, organs and organisms, by intentionally altering the expression of one or more of the identified genes.

Methods herein apply to extending the health and lifespan of human cells (and tissues, organs, and organisms), as well as cells (and tissues, organism, and organisms) of non-human animals, unicellular and multicellular organisms, plants, and so forth. Thus, it will be understood when a gene is referred to, that reference includes the orthologous sequence(s) from other species etc.

Healthy longevity includes causing cells to ‘offset’ age or environmental damage or disease, for instance related to decline in function (e.g., when mitochondria do not make as much ATP any longer, improving mitochondria respiration or increasing number of mitochondria or both addresses this), or reducing or eliminating expression/activity of an ‘unhealthy’ factor (e.g., MMP1 collagenase can be considered an unhealthy factor, as it degrades collagen which in turn damages the structural integrity of skin, joints, etc.). In this latter example, changing the ‘programming’ of gene expression improves health, for instance reducing or reversing the chronic response to injury (e.g., environmental or otherwise—UV light exposure, smoking, inflammation, etc.) that had caused/induced overproduction of MMP1, which prematurely ages cells and organs and organism.

The methods provided herein are useful also to modulate gene activity/expression in order to shorten lifespan of unhealthy cells, for instance in order to kill cancer or other unwanted cells or to eliminate cells that are sending ‘wrong’ genetic or molecular signals. When that is accomplished, you can replace the eliminated or down-modulated cells with cells that are healthy (e.g., through biogenesis or using stem cells). Alternatively, cells can be programmed to offset the negative signals—for instance, responding to overproduction of MMP1 by modulating the expression of a cell in order to produce collagen to replace that which the MMP1 is degrading.

The discoveries herein regarding gene expression in response to antioxidant induction provides a system that enables balancing of healthy and unhealthy influences to yield healthier longevity. The identified genes can be reprogrammed (either up or down, depending on the gene and the circumstance); where they are not amenable to reprogramming directly, the cell expressing them can be removed, incapacitated, killed (e.g., through apoptosis) or disabled; and where that is not readily feasible, other genes that counteract or balance the unhealthy influence(s) through offsetting expression of healthy factors, either in the same or another cell. Likewise, the counteracting influence may be biogenesis of new cells, repair of DNA damage, and/or prevention of DNA damage. All of these in different ways may be exploited to influence lifespan of cells, tissues, organs and organism—whether to extend lifespan or reduce it by causing lethal damage or triggering apoptosis is another way to get rid of cells.

The extracts, compounds or combination of compounds derived therefrom are prepared by methods commonly known and many naturally derived compounds are commercially available. Since naturally derived compounds are not the only way to achieve the concentrations of active compounds, the invention comprehends synthetic forms can be prepared from isolation from other plant species as well as from synthetic routes, which are all covered in these claims. Also the skilled artisan will be able to envision additional routes of synthesis, based on knowledge in the art, without undue experimentation. For instance, given the phenolic character of the compounds, variable methods of selective protection, coupled with organometallic additions, phenolic couplings and photochemical reactions, e.g., in a convergent, linear or biomimetic approach, together with standard well known reactions for synthetic organic chemists could produce synthetic derivatives that perform the desired telomeric length maintenance alterations.

Data from telomerase activity experiments show that green tea increases measurable activity of telomerase in most tested circumstances. Cells given green tea before UVB exposure show a decrease in telomerase activity. That is when the cells are stressed with UVB and given green tea, the measurable telomerase activity increases and when the cells are not stressed and contacted with green tea the telomerase activity also increases (younger cells are more responsive to the increase than older cells). Conversely, cells both stressed and unstressed, which receive treatment with idebenone show a decreased level of telomerase activity. These results indicate that by administering green tea to the cells, before stress, or even to normal unstressed cells, there is an increase in telomerase activity that is higher in the younger cell lines, indicating an increase in the ability to maintain the length of the telomere and to better protect against/combat oxidative stress based damage to the DNA, thereby reducing and/or preventing cellular damage and apoptosis signaling events.

In custom microarray experiments, the data illustrate that the chosen antioxidant compounds (green tea, coffee cherry and idebenone) are biologically active, showing statistically significant changes in expression levels for some longevity related genes in all the compounds tested. The 36 year old cells seem to be active with large statistical changes in the expression of PARP1, NADSYN1, IFI44, TERT, and NFKB1. All of these genes were downregulated when exposed to UVB stress, but upregulated when exposed to cells given the tested antioxidant compounds (for various time intervals) and then stressed. The increase in TERT (responsible for telomerase enzyme activity) compared to a decrease in the UVB alone (stressed) cells indicates very strongly that the antioxidant compounds are enabling more enzyme activity to keep telomere length intact (and thus extend the cellular lifespan) via the mediation of UV damage to the telomere. A second interesting finding is in older (presumably more environmentally damaged and less efficient at telomere repair and total telomere length) cells the idebenone and coffee cherry treated cells, and not green tea, were able to reverse the expression levels of UVB stressed aged cells. In this case, the TERT reduction from UVB stress was even greater, considering the age of the cell relative to the younger cell, but even more interestingly, the antioxidant response was even stronger than in the younger cell implying that, at least for idebenone and coffee cherry, the older/more damaged/less efficient the cellular mechanism of telomere length maintenance is, the greater the ability for certain antioxidants to effect changes towards longevity.

Green tea shows, through significant downregulation of TNF {18 fold}, a role in combating the damage caused by this pro-inflammatory cytokine, which could also lead to extending the lifespan of the cell by preventing apoptosis caused by inflammatory signaling cascades.

The RT-PCR primer assays examined genes specifically related to the telomere complex itself, and show that coffee cherry was able to downregulate the expression of TINF2 which, when expressed is a negative regulator of telomere length maintenance, and indicates a directional change toward the lengthening of telomeres and cellular longevity.

In Human Genome arrays the experiments show the effect of antioxidant activity (idebenone and coffee cherry) in stressed and unstressed cells on the entire genetic expression profile. The data showed both compounds to be biologically active and affecting many genes with a variety of functions focused on the aging/longevity groups and indicate the ability for the application of antioxidants to not only effect the free radical metabolism, but also directly affect gene expression profiles responsible for telomere length maintenance, cellular metabolism, mitochondrial function and inflammation all of which when modulated properly would lead to the potential extension of, and improvement in the quality of, cellular lifespan.

The application of the desired antioxidants, before UV stress or without UV stress, can directly affect the expression levels of genes responsible for telomere length maintenance and modulate the cellular lifespan. Telomere length maintenance is not the only factor involved in cell longevity, and antioxidants have shown the ability to modulate those expression levels as well, with effects on energy production and inflammation responses indicating multiple methods for extension of lifespan through a single antioxidant compound.

Based on the work presented herein, it is now recognized that that at least all of the genes listed in Table 1 and in Array 2 (described below) are involved in lifespan, longevity, mitochondrial biogenesis or health, cellular respiratory health, and/or DNA or telomere maintenance. Thus, it is contemplated that modification of the level or expression of any one or more of these genes may be useful in modulating such process. At least the following genes are therefore recognized as lifespan-influencing genes and useful in one or more of the methods and/or compositions described herein: 1553575_at, 1554007_at, 1554948_at, 1555846_a_at, 1555875_at, 1556097_at, 1556216_s_at, 1556242_a_at, 1556332_at, 1556545_at, 1556936_at, 1557118_a_at, 1557286_at, 1557287_at, 1557302_at, 1557341_x_at, 1557348_at, 1557383_a_at, 1557667_at, 1557740_a_at, 1558105_a_at, 1558236_at, 1558237_x_at, 1558250_s_at, 1558401_at, 1558445_at, 1558515_at, 1558604_a_at, 1558605_at, 1558750_a_at, 1558801_at, 1558836_at, 1558837_a_at, 1558890_at, 1558906_a_at, 1558920_at, 1559229_at, 1559867_at, 1560071_a_at, 1560208_at, 1560579_s_at, 1561064_a_at, 1562012_at, 1562013_a_at, 1562056_at, 1562098_at, 1562777_at, 1563012_x_at, 1563414_at, 1565495_at, 1565577_s_at, 1565783_at, 1568633_a_at, 1569129_s_at, 1569765_at, 1570061_at, 1570100_at, 182-FIP, 208187_s_at, 210230_at, 213156_at, 213158_at, 213567_at, 213817_at, 213832_at, 214202_at, 214808_at, 214862_x_at, 214967_at, 215128_at, 215287_at, 217166_at, 217536_x_at, 217540_at, 217554_at, 217604_at, 220494_s_at, 220726_at, 221200_at, 222184_at, 222284_at, 224769_at, 224778_s_at, 224811_at, 225256_at, 225356_at, 225725_at, 225893_at, 225917_at, 226203_at, 226250_at, 226282_at, 226316_at, 226362_at, 226365_at, 226392_at, 226457_at, 226458_at, 226520_at, 226532_at, 226542_at, 226546_at, 226550_at, 226773_at, 226885_at, 226964_at, 227041_at, 227044_at, 227051_at, 227061_at, 227082_at, 227121_at, 227126_at, 227184_at, 227193_at, 227221_at, 227252_at, 227283_at, 227306_at, 227422_at, 227503_at, 227531_at, 227533_at, 227565_at, 227623_at, 227655_at, 227663_at, 227682_at, 227929_at, 227955_s_at, 228032_s_at, 228045_at, 228049_x_at, 228084_at, 228156_at, 228159_at, 228216_at, 228242_at, 228304_at, 228315_at, 228333_at, 228346_at, 228390_at, 228478_at, 228528_at, 228694_at, 228740_at, 228742_at, 228750_at, 228773_at, 228781_at, 228811_at, 228812_at, 228850_s_at, 228955_at, 228963_at, 229024_at, 229072_at, 229121_at, 229189_s_at, 229190_at, 229281_at, 229297 at, 229315_at, 229319_at, 229333_at, 229359_at, 229384_at, 229460_at, 229479_at, 229512_at, 229569_at, 229572_at, 229602_at, 229615_at, 229641_at, 229699_at, 229705_at, 229710_at, 229756_at, 229757 at, 229795_at, 229810_at, 229815_at, 229948_at, 229994_at, 230003_at, 230090_at, 230127 at, 230183_at, 230211_at, 230227 at, 230240_at, 230304_at, 230345_at, 230383_x_at, 230406_at, 230407 at, 230431_at, 230446_at, 230449_x_at, 230483_at, 230503_at, 230683_at, 230741_at, 230766_at, 230773_at, 230860_at, 230927 at, 230968_at, 231055_at, 231069_at, 231193_s_at, 231238_at, 231576_at, 231597_x_at, 231890_at, 231963_at, 231993_at, 232088_x_at, 232125_at, 232156_at, 232484_at, 232535_at, 232538_at, 232656_at, 232795_at, 232903_at, 233105_at, 233335_at, 233354_at, 233376_at, 233485_at, 233518_at, 233723_at, 233814_at, 234340_at, 234578_at, 234723_x_at, 234983_at, 235000_at, 235028_at, 235046_at, 235072_s_at, 235078_at, 235123_at, 235124_at, 235171_at, 235207 at, 235224_s_at, 235227 at, 235264_at, 235279_at, 235299_at, 235302_at, 235304_at, 235352_at, 235407 at, 235427 at, 235428_at, 235434_at, 235438_at, 235459_at, 235532_at, 235556_at, 235571_at, 235581_at, 235585_at, 235612_at, 235655_at, 235658_at, 235696_at, 235733_at, 235761_at, 235764_at, 235830_at, 235831_at, 235889_at, 235890_at, 235919_at, 235931_at, 235938_at, 236004_at, 236038_at, 236089_at, 236097 at, 236105_at, 236174_at, 236180_at, 236194_at, 236196_at, 236335_at, 236344_at, 236350_at, 236364_at, 236423_at, 236433_at, 236452_at, 236619_at, 236685_at, 236787 at, 236856_x_at, 236875_at, 236898_at, 236922_at, 236996_at, 237071_at, 237212_at, 237290_at, 237315_at, 237416_at, 237435_at, 237622_at, 237768_x_at, 238030_at, 238050_at, 238109_at, 238178_at, 238250_at, 238308_at, 238360_s_at, 238389_s_at, 238431_at, 238456_at, 238463_at, 238483_at, 238501_at, 238557 at, 238559_at, 238565_at, 238573_at, 238576_at, 238604_at, 238617 at, 238620_at, 238673_at, 238684_at, 238712_at, 238733_at, 238766_at, 238782_at, 238824_at, 238861_at, 238890_at, 238932_at, 238934_at, 239066_at, 239218_at, 239231_at, 239266_at, 239278_at, 239331_at, 239370_at, 239503_at, 239543_s_at, 239669_at, 239708_at, 239710_at, 239842_x_at, 239845_at, 239847 at, 239866_at, 239951_at, 239973_at, 240020_at, 240095_at, 240128_at, 240165_at, 240190_at, 240219_at, 240233_at, 240366_at, 240418_at, 240523_at, 240549_at, 240557 at, 240885_at, 241114_s_at, 241263_at, 241359_at, 241484_x_at, 241689_at, 241721_at, 241722_x_at, 241815_at, 241823_at, 241838_at, 241863_x_at, 241879_at, 241887 at, 241925_x_at, 241936_x_at, 242005_at, 242051_at, 242107_x_at, 242134_at, 242289_at, 242312_x_at, 242321_at, 242323_at, 242358_at, 242366_at, 242376_at, 242421_at, 242471_at, 242486_at, 242523_at, 242606_at, 242719_at, 242818_x_at, 242845_at, 242979_at, 243115_at, 243179_at, 243278_at, 243302_at, 243366_s_at, 243404_at, 243417 at, 243489_at, 243551_at, 243564_at, 243606_at, 243641_at, 243671_at, 243680_at, 243697 at, 243707 at, 243844_at, 243907 at, 243925_at, 243934_at, 243947_s_at, 243976_at, 244007 at, 244025_at, 244032_at, 244242_at, 244271_at, 244350_at, 244354_at, 244533_at, 244663_at, 244677 at, 244701_at, 244779_at, 244853_at, 244855_at, 244864_at, 76P, A_{—}23_P10605, A_{—}23_P113263, A_{—}23_P113762, A_{—}23_P13202, A_{—}23_P134405, A_{—}23_P170719, A_{—}23_P205500, A_{—}23_P44053, A_{—}24_P136155, A_{—}24_P144054, A_{—}24_P195454, A_{—}24_P195621, A_{—}24_P229766, A_{—}24_P247303, A_{—}24_P289973, A_{—}24_P315674, A_{—}24_P3627, A_{—}24_P375360, A_{—}24_P384379, A_{—}24_P399341, A_{—}24_P41483, A_{—}24_P524164, A_{—}24_P607107, A_{—}24_P622375, A_{—}24_P626812, A_{—}24_P67268, A_{—}24_P682550, A_{—}24_P752362, A_{—}24_P7820, A_{—}24_P794833, A_{—}24_P799680, A_{—}24_P835943, A_{—}24_P84719, A_{—}24_P84738, A_{—}24_P913855, A_{—}24_P919931, A_{—}24_P922430, A_{—}24_P928031, A_{—}24_P942151, A_{—}32_P111919, A_{—}32_P135790, A_{—}32_P138933, A_{—}32_P149404, A_{—}32_P157622, A_{—}32_P190944, A_{—}32_P205522, A_{—}32_P220567, A_{—}32_P45087, A_{—}32_P71456, A_{—}32_P9931, A1BG, AA019203, AA043564, AA085955, AA344632, AA451708, AA581414, AA586832, AA631975, AA725860, AA918648, AADACL1, AARSD1, AASS, AB011149, ABCA1, ABCA5, ABCA6, ABCB10, ABCC3, ABCC6, ABCE1, ABCF2, ABHD11, ABHD13, ABHD2, ABHD3, ABHD5, ABHD8, ABI2, ABL2, ABTB2, ACACA, ACAD10, ACAD11, ACADS, ACADSB, ACBD4, ACE, ACLY, ACOT11, ACOT7, ACOT8, ACOX1, ACOX2, ACOX3, ACP2, ACRC, ACSBG1, ACSL3, ACSS3, ACTA2, ACTC1, ACTN1, ACTN4, ACTR2, ACTR3B, ACVR2A, ACVRL1, ADA, ADAL, ADAM10, ADAM19, ADAM32, ADAM33, ADAMS, ADAMTS1, ADAMTS2, ADAMTS5, ADAMTS6, ADAMTSL1, ADARB1, ADAT1, ADAT3, ADCY3, ADD1, ADDS, ADFP, ADH1B, ADH5, ADHFE1, ADK, ADM, ADORAL, ADORA2B, ADORA3, ADRA2A, ADRA2B, ADRA2C, ADRM1, AF034187, AF086017, AF086125, AF086187, AF086205, AF086329, AF146694, AF212044, AF334588, AFF3, AFG3L2, AFP, AFTPH, AGBL5, AGL, AGPAT3, AGPAT5, AGPAT6, AGRN, AGXT, AGXT2L2, AHCTF1, AHNAK, AHNAK2, AHR, AHRR, AHSA2, AI051172, AI161396, AI192327, AI263083, AI446524, AI457687, AI559980, AI652920, AI709405, AI873070, AI925475, AIFM2, AIM1, AJ295984, AK000053, AK001164, AK021467, AK021606, AK022044, AK022268, AK022339, AK022479, AK023328, AK023647, AK026418, AK026984, AK055302, AK074181, AK075186, AK092379, AK092668, AK093508, AK094296, AK094571, AK124806, AK126245, AK131472, AKAP1, AKAP11, AKAP12, AKAP8, AKNA, AKR1B10, AKR1C1, AKR1C2, AKR1C3, AKR1C4, AKR1CL2, AKT1, AKT2, AKT3, AKTIP, AL040873, AL041007, AL547361, AL566369, AL571926, AL833114, ALAS1, ALCAM, ALDH1B1, ALDH1L2, ALDH3A1, ALDH3A2, ALDH3B1, ALDH6A1, ALDH7A1, ALG10B, ALG11, ALG3, ALMS1, ALOX12, ALPK1, ALPK2, ALS2CR3, ALS2CR7, ALS2CR8, ALX3, AMAC1L2, AMDHD2, AMIGO2, AMOT, AMOTL1, AMPH, AMT, AMZ2, ANAPC5, ANAPC7, ANG, ANGEL2, ANGPTL2, ANGPTL4, ANK1, ANKDD1A, ANKFN1, ANKH, ANKIB1, ANKRA2, ANKRD10, ANKRD11, ANKRD12, ANKRD13C, ANKRD13D, ANKRD17, ANKRD26, ANKRD28, ANKRD29, ANKRD37, ANKRD38, ANKRD42, ANKRD44, ANKRD50, ANKRD57, ANKRD9, ANLN, ANP32D, ANP32E, ANTXR2, ANXA11, ANXA2P1, ANXA3, ANXA4, ANXA8L2, AOC2, AOF2, AOX1, AP1GBP1, AP1S1, AP2B1, AP4S1, APBB2, APCDD1, APCDD1L, APIS, APOBEC3B, APOBEC3C, APOBEC3F, APOBEC3G, APOD, APOE, APOL2, APOL3, APOL6, APOLD1, APOOL, APP, APPBP2, APPL1, AQP1, AQP10, ARC, ARD1A, ARF1, ARF3, ARF5, ARFGAP1, ARFRP1, ARG2, ARHGAP12, ARHGAP18, ARHGAP20, ARHGAP22, ARHGAP23, ARHGAP26, ARHGAP27, ARHGAP28, ARHGAP29, ARHGAP5, ARHGDIA, ARHGEF10L, ARHGEF12, ARHGEF17, ARHGEF3, ARHGEF6, ARHGEF7, ARID4A, ARID5B, ARIH1, ARL17, ARL5A, ARL61P1, ARL8B, ARMC7, ARMC9, ARMCX2, ARMCX3, ARMCX4, ARMET, ARNT, ARNT2, ARNTL, ARNTL2, ARPC4, ARPC5L, ARPP-19, ARRDC4, ARSD, ARSI, ARTS-1, ARVCF, ASAH2B, ASB6, ASF1B, ASPA, ASPH, ASPHD1, ASPM, ASPN, ASS1, ASXL1, ATAD2, ATAD2B, ATAD3A, ATAD3B, ATF3, ATF6, ATF71P, ATM, ATOH8, ATP10A, ATP11B, ATP1A2, ATP1B3, ATP2A1, ATP2B1, ATP2B3, ATP2C1, ATP5C1, ATP5S, ATP6V0B, ATP6V0D2, ATP6V0E, ATP6V0E1, ATP6V0E2, ATP6V1C1, ATP6V1C2, ATP6V1G2, ATP6V1H, ATP7A, ATP8A2, ATP8B1, ATPBD1C, ATRIP, ATRX, ATXN1, ATXN1L, ATXN3, ATXN7L1, AUP1, AURKA, AURKB, AV702101, AW167080, AW191706, AW858928, AW885990, AW901755, AXIN2, AXUD1, AZI2, AZIN1, B3GALNT1, B3GALT2, B3GALT4, B3GALT6, B3GAT3, B4GALNT1, B4GALT1, B9D1, BAALC, BACE1, BAG1, BAG2, BAG4, BAIAP2, BAIAP2L1, BAK1, BAMBI, BANK1, BAP1, BATT, BAT2D1, BAX, BBS1, BBS2, BBS9, BC008476, BC015334, BC015449, BC019907, BCO32901, BCO36599, BCO36928, BC047110, BCAP31, BCAR3, BCAR4, BCAS3, BCHE, BCKDHB, BCL11A, BCL2, BCL2L1, BCL2L11, BCL2L12, BCL6, BCL7A, BCLAF1, BCR, BDH2, BDKRB1, BDKRB2, BDNF, BE379389, BE644757, BE719776, BE766438, BEX1, BEXL1, BF195626, BHLHB2, BHLHB3, BI836739, BICC1, BICD1, BIRC4, BIRC5, BIVM, BLCAP, BLM, BLOC1S1, BLOC1S3, BMO23, BM455859, BM986990, BMP2, BMP2K, BMP6, BMPER, BMPR1B, BMPR2, BNC1, BNC2, BNIP2, BOC, BOK, BOLA2, BOP1, BP872463, BPGM, BPI, BPTF, BQ000605, BQ772270, BRAP, BRD2, BRD4, BRD8, BRF2, BRIP1, BRMS1L, BRP44, BRWD1, BTBD14A, BTBD2, BTBD7, BTD, BTG1, BTG3, BTN3A1, BTN3A2, BTN3A3, BU160948, BU561469, BUB1, BUB1B, BYES, BX098411, BX100298, BX412469, BX433326, BX448200, BXDC2, BYSL, C10orf10, C10orf104, C10orf107, C10orf11, C10orf118, C10orf119, C10orf125, C10orf140, C10orf18, C10orf27, C10orf32, C10orf33, C10orf39, C10orf46, C10orf54, C10orf56, C10orf57, C10orf59, C10orf72, C10orf90, C10orf91, C11orf17, C11orf21, C11orf30, C11orf31, C11orf32, C11orf34, C11orf41, C11orf46, C11orf48, C11orf54, C11orf57, C11orf70, C11orf73, C11orf82, C12orf30, C12orf41, C12orf42, C12orf48, C12orf49, C12orf56, C13orf1, C13orf15, C13orf18, C13orf3, C13orf31, C13orf33, C14orf1, C14orf101, C14orf115, C14orf122, C14orf132, C14orf133, C14orf138, C14orf139, C14orf145, C14orf159, C14orf167, C14orf172, C14orf173, C14orf43, C14orf49, C14orf80, C15orf23, C15orf33, C15orf38, C15orf40, C15orf41, C15orf48, C15orf5, C15orf52, C16orf44, C16orf45, C16orf53, C16orf55, C16orf57, C16orf59, C16orf61, C16orf63, C16orf72, C16orf80, C17orf32, C17orf39, C17orf44, C17orf63, C17orf67, C18orf17, C18orf19, C18orf24, C18orf25, C18orf37, C18orf50, C18orf54, C18orf56, C19orf24, C19orf25, C19orf28, C19orf48, C19orf6, C19orf61, C1GALT1, C1orf104, C1orf107, C1orf108, C1orf112, C1orf116, C1orf128, C1orf133, C1orf135, C1orf144, C1orf163, C1orf165, C1orf175, C1orf192, C1orf198, C1orf201, C1orf21, C1orf213, C1orf216, C1orf217, C1orf25, C1orf43, C1orf46, C1orf51, C1orf55, C1orf56, C1orf58, C1orf63, C1orf69, C1orf71, C1orf75, C1orf77, C1orf9, C1orf93, C1orf96, C1QTNF4, C1RL, C1S, C20orf108, C20orf111, C20orf165, C20orf177, C20orf19, C20orf198, C20orf23, C20orf3, C20orf59, C20orf7, C20orf74, C21orf114, C21orf2, C21orf34, C21orf45, C21orf51, C21orf74, C21orf86, C21orf91, C22orf24, C22orf9, C2orf18, C2orf3, C2orf34, C2orf49, C2orf60, C2orf7, C3, C3orf26, C3orf33, C3orf34, C3orf52, C3orf63, C4orf12, C4orf15, C4orf23, C4orf32, C4orf34, C5orf13, C5orf16, C5orf23, C5orf24, C5orf34, C5orf4, C6orf105, C6orf107, C6orf117, C6orf128, C6orf129, C6orf130, C6orf151, C6orf155, C6orf166, C6orf170, C6orf173, C6orf199, C6orf204, C6orf206, C6orf32, C6orf5, C6orf62, C6orf65, C6orf66, C6orf85, C6orf89, C7orf10, C7orf19, C7orf20, C7orf24, C7orf29, C7orf38, C7orf40, C7orf41, C7orf51, C7orf53, C7orf55, C8orf31, C8orf4, C8orf47, C8orf61, C8orf66, C9orf100, C9orf127, C9orf130, C9orf139, C9orf140, C9orf151, C9orf152, C9orf32, C9orf39, C9orf44, C9orf5, C9orf52, C9orf53, C9orf72, C9orf85, C9orf89, CA13, CA503034, CA5B, CA772440, CA843452, CA866957, CABC1, CABYR, CACHD1, CACNA1A, CACNA1C, CACNA2D1, CADM1, CADPS2, CALB2, CALD1, CALML4, CAMK1, CAMK2D, CAMK2G, CAMKK1, CAND2, CANT1, CAPG, CAPS, CAPS2, CAPZA1, CAPZ9, CARD10, CARD8, CARD9, CARKL, CASC2, CASC4, CASC5, CASD1, CASP1, CASP2, CASP9, CAST, CAT, CAV2, CB984746, CBFA2T2, CBFB, CBLL1, CBLN3, CBR4, CBS, CBX4, CBX5, CBX6, CC2D2A, CCBE1, CCDC102A, CCDC113, CCDC115, CCDC123, CCDC124, CCDC13, CCDC131, CCDC134, CCDC136, CCDC137, CCDC15, CCDC16, CCDC18, CCDC19, CCDC32, CCDC34, CCDC50, CCDC69, CCDC74B, CCDC75, CCDC77, CCDC85B, CCDC86, CCDC88A, CCDC89, CCDC91, CCDC93, CCDC98, CCDC99, CCK, CCL2, CCL26, CCL4L1, CCNA2, CCNB1, CCNB2, CCND1, CCND3, CCNE1, CCNE2, CCNF, CCNG1, CCNG2, CCNL2, CCPG1, CCR2, CCRL1, CCRN4L, CCT5, CD109, CD1C, CD24, CD274, CD276, CD28, CD302, CD34, CD3EAP, CD44, CD47, CD55, CD69, CD9, CDA, CDA08, CDC14B, CDC2, CDC20, CDC23, CDC25A, CDC25C, CDC2L1, CDC2L5, CDC2L6, CDC42BPA, CDC42EP1, CDC42EP2, CDC42EP3, CDC45L, CDC6, CDCA2, CDCA3, CDCA4, CDCA5, CDCA7, CDCA8, CDCP1, CDH11, CDH4, CDH7, CDIPT, CDK2, CDK2AP2, CDK3, CDK5R1, CDK5RAP2, CDK5RAP3, CDK6, CDKL1, CDKN1B, CDKN1C, CDKN2A, CDKN2C, CDKN3, CDON, CDR2L, CDRT15, CDT1, CDYL, CEBPD, CECR1, CECR2, CELSR1, CENPA, CENPE, CENPF, CENPI, CENPJ, CENPK, CENPL, CENPM, CENPN, CENPO, CENPQ, CENPT, CENTD2, CENTG3, CEP152, CEP170, CEP290, CEP350, CEP55, CEP57, CEP68, CEP70, CEP76, CEP78, CERK, CES2, CFB, CFD, CFDP1, CFH, CFL1, CGB, CGGBP1, CGNL1, CH25H, CHAC2, CHAF1A, CHAF1B, CHD6, CHDH, CHKA, CHM, CHMP1B, CHN1, CHP, CHRNA9, CHST11, CHST2, CHST3, CHST6, CHSY-2, CHTF18, CILP, CINP, CIP29, CIR, CIRBP, CITED2, CITED4, CK818527, CKAP2, CKAP2L, CKLF, CKS1B, CKS2, CLCC1, CLCF1, CLCN5, CLDN11, CLDND1, CLEC2B, CLEC3B, CLIP1, CLIPS, CLIP4, CLK1, CLK4, CLN5, CLN6, CLN8, CLP1, CLPB, CLSPN, CLTC, CLU, CLUAP1, CMAH, CMBL, CMTM7, CNDP2, CNIH3, CNKSR2, CNKSR3, CNN1, CNNM2, CNOT3, CNOT6L, CNPY3, CNTN3, COCH, COG6, COIL, COL12A1, COL14A1, COL15A1, COL1A1, COL1A2, COL27A1, COL3A1, COL4A3BP, COL5A2, COL6A1, COL6A6, COL8A1, COL9A1, COLEC12, COMMD10, COMMD6, COP1, COPG, COPS7B, COQ10B, COTL1, COX1, COX17, CPD, CPEB1, CPEB2, CPEB3, CPEB4, CPNE4, CPSF3L, CPSF6, CPT1C, CR598370, CR605947, CR616772, CR617865, CR740121, CRABP2, CRBN, CREB1, CREB3L2, CREBBP, CREBZF, CREG2, CREM, CRIM1, CRIPAK, CRISPLD2, CRKRS, CROCCL2, CRP, CRSP8, CRTC2, CRY1, CRY2, CRYZ, CRYZL1, CSE1L, CSF2RB, CSNK1E, CSPG4, CSRP1, CSRP2, CST6, CSTA, CTA-126B4.3, CTB-1048E9.5, CTBS, CTDSP2, CTDSPL, CTDSPL2, CTGF, CTNS, CTPS, CTSC, CTSD, CTSF, CTSO, CTSS, CTTN, CUEDC2, CUGBP1, CUGBP2, CUL4A, CUL5, CULT, CUTC, CV326037, CXCL2, CXCL3, CXCL5, CXCL6, CXorf23, CXorf38, CXorf39, CXorf45, CXorf6, CXXC6, CYB561D2, CYB5D2, CYB5R3, CYBRD1, CYCS, CYFIP2, CYLD, CYP19A1, CYP1B1, CYP20A1, CYP26B1, CYP2C18, CYP2U1, CYP4V2, CYR61, CYST, CYYR1, D15Wsu75e, D31825, D90075, DAAM1, DAAM2, DAB2, DACT1, DAGLA, DAGLB, DALRD3, DAP, DAPK2, DB318193, DB318210, DB352368, DBF4, DBNL, DBP, DBT, DCBLD1, DCBLD2, DCHS1, DCK, DCLK1, DCLK3, DCLRE1B, DCN, DCP_{—}1_{—}7, DCP_{—}22_{—}0, DCP_{—}22_{—}2, DCP22_{—}4, DCP22_{—}6, DCP22_{—}7, DCP_{—}22_{—}9, DCP1A, DCP1B, DCTN5, DDA1, DDAH1, DDAH2, DDB2, DDC, DDEF1IT1, DDEF2, DDEFL1, DDHD1, DDHD2, DDI2, DDIT4L, DDOST, DDR1, DDR2, DDX17, DDX19A, DDX21, DDX24, DDX39, DDX3X, DDX46, DDX50, DDX51, DDX54, DDX55, DEDD2, DENND1A, DENND1B, DENND2A, DENND2D, DENND4A, DENND4C, DEPDC1, DEPDC1B, DEPDC2, DFFA, DFNB59, DGKA, DGKE, DHCR24, DHDH, DHFR, DHFRL1, DHRS1, DHRS12, DHRS2, DHRS7B, DHTKD1, DHX30, DHX37, DHX40, DIAPH1, DIAPH2, DIAPH3, DICER1, DIP2A, DIRAS3, DIS3L, DISP1, DKFZp434A0530, DKFZP434B0335, DKFZp434C198, DKFZp434D193, DKFZp434G0514, DKFZp434H0350, DKFZp434H1419, DKFZp434H152, DKFZp4341062, DKFZP43410714, DKFZp434J1521, DKFZp434L1123, DKFZp434N1010, DKFZp434N2435, DKFZp434P1735, DKFZP434P211, DKFZp451A211, DKFZp547E087, DKFZp564A063, DKFZp564CO482, DKFZp564F1862, DKFZP564J0863, DKFZp564J1864, DKFZp564K2364, DKFZp564L2362, DKFZP56400523, DKFZp566K1946, DKFZp586H1322, DKFZp586J1119, DKFZP586K1520, DKFZp667E0512, DKFZp667G2110, DKFZp761C121, DKFZp761D221, DKFZp761F0123, DKFZp761I172, DKFZp761J17121, DKFZp761M0423, DKFZP761M1511, DKFZp761P0423, DKFZp762B2310, DKK2, DLC1, DLEU2, DLEU2L, DLG2, DLG5, DLG7, DLX1, DLX2, DMD, DMXL1, DNAH2, DNAJA4, DNAJA5, DNAJB1, DNAJB14, DNAJB2, DNAJB5, DNAJB9, DNAJC18, DNAJC3, DNAJC5, DNAJC9, DNAL1, DNALI1, DNASE2, DNM1, DNM1L, DNM3, DNPEP, DOCK1, DOCK11, DOCKS, DOCK4, DOCK5, DOHH, DOT1L, DPF3, DPH2, DPH3, DPH5, DPM2, DPP4, DPP8, DPT, DPY19L1, DPY19L1P1, DPY19L4, DPYD, DPYSL2, DPYSL3, DSEL, DSN1, DTL, DTNA, DTWD1, DTWD2, DTX3L, DTYMK, DUSP1, DUSP10, DUSP13, DUSP14, DUSP16, DUSP18, DUSP2, DUSP23, DUSP3, DUSP4, DUSP5, DUSP6, DUSP8, DYM, DYNC1H1, DYNC2H1, DYNC2L11, DYNLL2, DYRK1A, DYRK3, DYSF, DZIP1, DZIP3, E2F1, E2F7, E2F8, E4F1, EBF, EBF1, EBF2, EBI2, ECE2, ECH1, ECHDC2, ECM2, EDC3, EDEM1, EDG1, EDG2, EDG5, EDNRA, EEA1, EEF2K, EFCAB2, EFCAB4B, EFCAB6, EFHA2, EFHC1, EFHD2, EFNA4, EFNB2, EFNB3, EGF, EGFR, EGR1, EGR2, EGR3, EHBP1, EHD1, EHD3, EHD4, EHF, EID3, EIF1, EIF2C1, EIF2C4, EIF2S1, EIF2S2, EIF2S3, EIF3A, EIF3S9, EIF4A2, EIF4A3, EIF4B, EIF4E, EIF4E3, EIF4EBP2, EIF4G1, EIF5A2, ELK4, ELL2, ELL3, ELMO2, ELMOD1, ELMOD2, ELOVL6, EMG1, EML2, EML4, EMP1, EMX2, EMX20S, ENAH, ENC1, ENDOD1, ENDOG, ENG, ENO1B, ENO2, ENOX1, ENPP2, ENST00000256861, ENST00000302942, ENST00000306515, ENST00000327781, ENST00000342829, ENST00000354343, ENST00000356104, ENST00000366930, ENST00000366971, ENST00000371408, ENST00000379108, ENST00000379131, ENST00000380357, ENTPD7, EP300, EPAS1, EPB41, EPB41L1, EPB41L2, EPB41L3, EPB41L4B, EPB41L5, EPHA2, EPHA4, EPHB1, EPHB4, EPOR, EPPB9, EPR1, EPRS, EPSTI1, ERBB2IP, ERBB3, ERCC1, ERCC2, ERCC4, ERF, ERGIC2, ERMAP, ERMP1, ERN1, ERRFI1, ESCO2, ESM1, ESPL1, ETAA1, ETNK1, ETS1, ETS2, ETV1, ETV4, ETV5, EVC, EVC2, EVIL, EVI2A, EVI2B, EVI5, EVL, EWSR1, EXO1, EXOC3, EXOC4, EXOSC2, EXOSC4, EXOSC5, EXOSC6, EXT1, EYA2, EZH1, EZH2, F10, F2RL1, F2RL3, F3, FABP5, FABP6, FAM100A, FAM100B, FAM102B, FAM105B, FAM108C1, FAM110B, FAM112A, FAM113A, FAM114A1, FAM115A, FAM120B, FAM122A, FAM122C, FAM126A, FAM126B, FAM129A, FAM130A1, FAM133A, FAM134B, FAM135A, FAM13A1, FAM13C1, FAM20A, FAM21C, FAM27A, FAM29A, FAM3A, FAM40B, FAM44A, FAM46A, FAM54A, FAM55C, FAM58A, FAM60A, FAM62B, FAM63A, FAM63B, FAM64A, FAM65A, FAM72A, FAM73A, FAM73B, FAM76A, FAM83D, FAM83G, FAM83H, FAM84A, FAM84B, FAM87A, FAM8A1, FAM92A1, FANCA, FANCF, FANCG, FANCI, FAP, FARP1, FARP2, FARSA, FASN, FASTKD5, FAT4, FBLN1, FBLN2, FBLN7, FBN2, FBXL14, FBXL17, FBXL20, FBXL4, FBXL6, FBXL7, FBXO16, FBXO30, FBXO31, FBXO32, FBXO33, FBXO45, FBXO9, FBXW2, FBXW7, FCMD, FDPSL2A, FDXR, FECH, FEN1, FER1L3, FGF1, FGF18, FGF5, FGFR1OP, FGFR3, FGFRL1, FHL1, FHOD1, FIBCD1, FIBIN, FIG4, FIGNL1, FILIP1L, FIP1L1, FIST, FJX1, FKBP1A, FKBP4, FKBP5, FKBPL, FKSG12, FKSG24, FLJ10292, FLJ10357, FLJ10769, FLJ10815, FLJ10986, FLJ10996, FLJ11000, FLJ11151, FLJ11286, FLJ11736, FLJ11806, FLJ11996, FLJ13231, FLJ14213, FLJ20030, FLJ20035, FLJ20309, FLJ20433, FLJ21616, FLJ21777, FLJ21986, FLJ22639, FLJ22659, FLJ22662, FLJ23556, FLJ23754, FLJ23861, FLJ23867, FLJ25006, FLJ25328, FLJ25778, FLJ27365, FLJ30851, FLJ31306, FLJ31401, FLJ32679, FLJ33674, FLJ33996, FLJ34208, FLJ35348, FLJ35379, FLJ36701, FLJ38348, FLJ38717, FLJ38973, FLJ39051, FLJ39653, FLJ39660, FLJ40113, FLJ40142, FLJ40330, FLJ42393, FLJ42709, FLJ43276, FLJ43663, FLJ43692, FLJ44342, FLJ44635, FLJ90757, FLNA, FLNB, FLNC, FLOT1, FLRT3, FLT3LG, FLYWCH2, FMNL1, FMNL2, FMNL3, FN1, FNBP1, FNDC3B, FNIP1, FOLR3, FOS, FOSB, FOSL1, FOSL2, FOXC1, FOXC2, FOXL2, FOXO1, FOXO3, FOXO3A, FOXO4, FOXP1, FOXQ1, FOXRED2, FPGT, FREQ, FRMD3, FRMD4A, FRMPD4, FRY, FSCN1, FSHPRH1, FST, FSTL3, FTHL16, FUBP1, FURIN, FUS, FUT1, FUT4, FVT1, FXR1, FXR2, FYCO₁, FYN, FZD5, FZD8, GOS2, G3BP1, G6PC3, GAB1, GABBR1, GABBR2, GABPA, GABPB2, GABRA2, GABRB3, GABRE, GADD45A, GADD45B, GAFA1, GAK, GAL, GAL3ST4, GALE, GALM, GALNT12, GALNT4, GALNT5, GALNTL2, GALT, GANAB, GART, GAS1, GAS2L3, GAS7, GATA2, GATA6, GATAD1, GATAD2B, GBA2, GBP2, GBP3, GCC2, GCH1, GCL, GCLC, GCLM, GCN1L1, GCNT1, Gcoml, GCS1, GDF15, GDF5, GDF6, GDPD1, GEM, GEMIN4, GEMIN8, GFOD1, GFPT2, GGA2, GGCX, GGT1, GH1, GHR, GIMAP2, GINS1, GINS2, GINS3, GINS4, GIPC2, GIPR, GIYD2, GJA1, GJA7, GJC1, GK, GK3P, GK5, GKAP1, GLA, GLCCl1, GLG1, GLI3, GLIPR1, GLIPR1L2, GLIS2, GLIS3, GLS, GLT8D2, GLT8D4, GM2A, GMEB1, GMFB, GMNN, GMPPB, GMPS, GNAI1, GNAQ, GNAT1, GNB4, GNG2, GNPAT, GNPDA1, GNS, GOLGA1, GOLGA2, GOLGA2LY1, GOLGA3, GOLGA4, GOLGA8A, GOLGA8B, GOLGB1, GOLT1B, GOSR2, GPATCH4, GPC4, GPC6, GPER, GPR1, GPR120, GPR124, GPR132, GPR133, GPR137c, GPR153, GPR157, GPR161, GPR172A, GPR177, GPR56, GPRASP1, GPRC5A, GPRC5C, GPSM2, GPT2, GRAMD1B, GRAMD3, GRB10, GRB14, GRB2, GRIA1, GRIA3, GRIK2, GRIPAP1, GRK5, GRLF1, GRM4, GRM5, GRPEL1, GRTP1, GSC2, GSDMDC1, GSK3B, GSN, GSPT1, GSR, GSTM1, GSTM2, GSTM4, GSTM5, GSTO1, GTDC1, GTF3C4, GTPBP10, GTPBP2, GTPBP5, GTSE1, GULP1, GUSBL2, GYPC, GYS1, H2AFV, H2AFX, H₂BFS, H40632, H43551, H6PD, HAB1, HABP4, HACE1, HADHA, HAL, HAPLN1, HAPLN3, HARS, HAS2, HAT1, HBEGF, HCCS, HCFC1R1, hCG_{—}1730474, hCG_{—}1776047, hCG_{—}1806964, hCG_{—}1985469, hCG_{—}20426, HCG11, HCP5, HDAC11, HDAC4, HDAC9, HEATR1, HEATR5A, HECW2, HEG1, HELLS, HELZ, HEPH, HERC3, HERPUD2, HEST, HES4, HESX1, HEXIM2, HEY1, HFE, HFL@, HGSNAT, HHLA3, HIBADH, HIC2, HIP1, HIP2, HIPK2, HIRIP3, HIST1H1A, HIST1H₁C, HIST1H1D, HIST1H1E, HIST1H2AB, HIST1H2AD, HIST1H2AE, HIST1H2AG, HIST1H2AI, HIST1H2AM, HIST1H₂BB, HIST1H₂BC, HIST1H₂BD, HIST1H₂BE, HIST1H₂BF, HIST1H₂BG, HIST1H₂BH, HIST1H₂BI, HIST1H₂BJ, HIST1H₂BK, HIST1H₂BM, HIST1H₂BO, HIST1H3A, HIST1H₃B, HIST1H3D, HIST1H₃F, HIST1H3G, HIST1H3H, HIST1H₄B, HIST1H₄C, HIST1H4D, HIST1H4E, HIST1H4H, HIST1H4J, HIST1H4K, H1ST2H2AA, H1ST2H2AA3, HIST2H₂BE, HIST2H4, H1ST3H2A, HK2, HLA-DRA, HLCS, HLX, HM13, HMBOX1, HMCN1, HMG20B, HMGA2, HMGB1, HMGB2, HMGB3, HMGCR, HMMR, HMOX1, HN1, HNMT, HNRNPA2B1, HNRNPL, HNRNPR, HNRNPU, HNRPAB, HNRPD, HNRPH3, HNRPLL, HNT, HOMER1, HOMER2, HOOKS, HOXA1, HOXA10, HOXA11, HOXA13, HOXA2, HOXA3, HOXB3, HOXB5, HOXB6, HOXB7, HOXC6, HOXC9, HOXD4, HOXD9, HP1BP3, HPCAL1, hqp0376 protein, HRAS, HRASLS, HS2ST1, HS3ST2, HS3ST3B1, HSDL2, HSP90AB1, HSP90AB3P, HSPA12A, HSPA14, HSPA1A, HSPA1B, HSPA2, HSPA5, HSPA6, HSPB3, HSPB7, HSPB8, HSPC047, HSPC111, HSPC171, HSPC173, HSPC180, HSPC252, HSPD1, HSPG2, HSPH1, HTATIP2, HTR2A, HTR7, HUNK, HUS1, HUWE1, HYLS1, HYOU1, HYPE, IAH1, IARS, IBRDC3, ICA1L, ICK, ID1, ID2, ID3, ID4, IDH1, IDH3A, IDH3B, IDS, IER2, IER3, IFI27, IFI44, IFIT1, IFIT2, IFIT3, IFNAR1, IFRD2, IFT52, IFT57, IFT74, IFT80, IFT81, IGF1, IGF2, IGF1R, IGFBP5, IGHG1, IGJ, IGLL1, IGSF9, 1HPK2, IKZF2, IKZF4, IKZF5, IL10, IL11, IL15, IL17RB, IL17RC, IL17RD, IL1A, IL1R1, IL1RAP, IL1RN, IL2, IL20RB, IL21R, IL24, IL27RA, IL32, IL33, IL4R, IL6, IL6R, IL6ST, IL7, IL7R, IL8, ILF3, IMMP1L, IMMT, IMP4, IMPACT, IMPAD1, INADL, ING1, ING4, INHBA, INHBB, INPP4B, INSIG1, INTS10, INTS6, IPO4, IPO9, IQCE, IQGAP3, IQSEC1, IRAK1BP1, IRAK2, IREB2, IRF2, IRF2BP2, IRS1, ISG20, ISG20L1, ITCH, ITGA2, ITGA3, ITGA4, ITGA5, ITGA6, ITGAV, ITGB2, ITGB3, ITGB8, ITGBL1, ITIH5, ITPR2, ITPR3, ITSN1, ITSN2, JARID1A, JARID1C, JARID2, JAZF1, JHDM1D, JMJD2C, JMJD3, JMJD4, JMJD6, JMY, JRK, JUB, JUN, JUNB, JUP, KATNAL2, KBTBD2, KBTBD3, KBTBD7, KBTBD8, KCNAB1, KCNC4, KCNE1, KCNE3, KCNE4, KCNG1, KCNH2, KCNJ15, KCNJ2, KCNK1, KCNK2, KCNMB2, KCNN4, KCNQ5, KCNS2, KCNS3, KCTD11, KCTD12, KCTD14, KCTD17, KCTD4, KCTD5, KGFLP1, KIAA0020, KIAA0090, KIAA0101, KIAA0182, KIAA0194, KIAA0226, KIAA0232, KIAA0241, KIAA0247, KIAA0256, KIAA0265, KIAA0280, KIAA0372, KIAA0409, KIAA0427, KIAA0513, KIAA0528, KIAA0556, KIAA0664, KIAA0802, KIAA0892, KIAA0913, KIAA0922, KIAA0974, KIAA0999, KIAA1107, KIAA1109, KIAA1143, KIAA1199, KIAA1217, KIAA1267, KIAA1276, KIAA1305, KIAA1333, KIAA1370, KIAA1377, KIAA1407, KIAA1430, KIAA1432, KIAA1450, KIAA1462, KIAA1467, KIAA1524, KIAA1545, KIAA1546, KIAA1549, KIAA1598, KIAA1609, KIAA1632, KIAA1641, KIAA1648, KIAA1683, KIAA1704, KIAA1715, KIAA1729, KIAA1731, KIAA1751, KIAA1754, KIAA1799, KIAA1833, KIAA1908, KIAA1913, KIAA1919, KIAA1946, KIAA1958, KIAA1967, KIAA2018, KIF11, KIF14, KIF15, KIF18A, KIF20A, KIF22, KIF23, KIF24, KIF26A, KIF2A, KIF2C, KIF3A, KIF3B, KIF4A, KIF5A, KIF6, KIFC1, KIFC3, KIR3DL1, KIRREL3, KIT, KITLG, KL, KLC1, KLC3, KLC4, KLF1, KLF10, KLF12, KLF16, KLF2, KLF7, KLF8, KLHDC1, KLHDC4, KLHDC5, KLHDC8B, KLHDC9, KLHL11, KLHL17, KLHL18, KLHL20, KLHL21, KLHL24, KLHL28, KLHL5, KLHL7, KPNA4, KPNA5, KRAS, KREMEN1, KRIT1, KRT14, KRT15, KRT16, KRT18, KRT19, KRT33A, KRT33B, KRT34, KRT7, KRT73, KRT78, KRT80, KRT81, KRTAP1-5, KRTAP4-10, Kua, KY, L3 MBTL, LAMA2, LAMA4, LAMB1, LAMC2, LARP2, LARP5, LARS, LASS6, LBA1, LBH, LCAT, LCE3D, LCORL, LDB1, LDB2, LDLR, LENG8, LEPR, LEPREL1, LETM1, LETM2, LETMD1, LFNG, LGALS8, LGALS9, LGI2, LGI4, LGR4, LHB, LHCGR, LHX9, LIFR, LIG1, LIMA1, LIMCH1, LIMK1, LIMS1, LIMS2, LIMS3, LIN28B, LIN7B, LIN9, LIPC, LIPT1, LIX1L, LMCD1, LMNA, LMO4, LMOD1, LNPEP, LNX1, LOC100049076, LOC113179, LOC128977, LOC130074, LOC137886, LOC144874, LOC146346, LOC146909, LOC147343, LOC147650, LOC147727, LOC149478, LOC149773, LOC151162, LOC152217, LOC152485, LOC152742, LOC153222, LOC153457, LOC153682, LOC158257, LOC158402, LOC201164, LOC201175, LOC201229, LOC201895, LOC203107, LOC220077, LOC220594, LOC220729, LOC221091, LOC221710, LOC222070, LOC222159, LOC23117, LOC253039, LOC254128, LOC255480, LOC255512, LOC257396, LOC25845, LOC283075, LOC283357, LOC283378, LOC283508, LOC283551, LOC283658, LOC283666, LOC283788, LOC283871, LOC283874, LOC283951, LOC284058, LOC284072, LOC284323, LOC284356, LOC284371, LOC284801, LOC285086, LOC285535, LOC285550, LOC285831, LOC285835, LOC285923, LOC286044, LOC286052, LOC286144, LOC286161, LOC286167, LOC286170, LOC286437, LOC338328, LOC338758, LOC339483, LOC339692, LOC344887, LOC346887, LOC348174, LOC348801, LOC374491, LOC387647, LOC387763, LOC388180, LOC388237, LOC388388, LOC388480, LOC388526, LOC388620, LOC388727, LOC388890, LOC388969, LOC389025, LOC389072, LOC389102, LOC389129, LOC389440, LOC389517, LOC389831, LOC389834, LOC390533, LOC390861, LOC391426, LOC392271, LOC392454, LOC399786, LOC399818, LOC399947, LOC399959, LOC400047, LOC400464, LOC400581, LOC400642, LOC400752, LOC401020, LOC401022, LOC401074, LOC401216, LOC401317, LOC401384, LOC401394, LOC401504, LOC401537, LOC402778, LOC439911, LOC439962, LOC440061, LOC440104, LOC440135, LOC440248, LOC440354, LOC440426, LOC440434, LOC440472, LOC440731, LOC440836, LOC440853, LOC440900, LOC440993, LOC440995, LOC441108, LOC441190, LOC441207, LOC441208, LOC441455, LOC441468, LOC441778, LOC442013, LOC442240, LOC442245, LOC442367, LOC442370, LOC492311, L0051152, L0051581, L00541471, L00550643, L00554202, L00554203, L0056757, L00595101, LOC63920, LOC641298, LOC641999, LOC642398, LOC642580, LOC642852, LOC643072, LOC643517, LOC643641, LOC643650, LOC643668, LOC643837, LOC644053, LOC644192, LOC644215, LOC644353, LOC645233, LOC645238, LOC645431, LOC645561, LOC645634, LOC645676, LOC646371, LOC646450, LOC646561, LOC646590, LOC646626, LOC646762, LOC647087, LOC647190, LOC647305, LOC647859, LOC647946, LOC647979, LOC648269, LOC648498, LOC650766, LOC650794, LOC652968, LOC653256, LOC653562, LOC653877, LOC654779, LOC727773, LOC727820, LOC727893, LOC727942, LOC728198, LOC728264, LOC728285, LOC728448, LOC728499, LOC728555, LOC728661, LOC728730, LOC729013, LOC729082, LOC729124, LOC729222, LOC729392, LOC729436, LOC729446, LOC729570, LOC729678, LOC729839, LOC730057, LOC730101, LOC730102, LOC730202, LOC730259, LOC730421, LOC731059, LOC731484, LOC731848, LOC731884, LOC90586, LOC91137, LOC91461, LOC92017, LOC92482, LOC93349, LOH11CR2A, LOH3CR2A, LONP2, LOXL4, LPIN1, LPP, LPPR2, LPXN, LRAP, LRBA, LRCH3, LRFN4, LRIG1, LRIG2, LRIG3, LRIT1, LRP1, LRP11, LRP12, LRP5L, LRP6, LRP8, LRPPRC, LRRC16, LRRC17, LRRC2, LRRC23, LRRC27, LRRC28, LRRC34, LRRC37A, LRRC37A2, LRRC37B2, LRRC44, LRRC51, LRRC58, LRRC61, LRRC8A, LRRC8C, LRRC8E, LRRFIP1, LRRFIP2, LRRIQ2, LRRK1, LRRK2, LRWD1, LSR, LSS, LTA4H, LTB4DH, LTB4R, LTB4R2, LTBP4, LUM, LY6E, LY6G5C, LY6K, LYAR, LYN, LYPD1, LYPD3, LYPD6, LYPLA2, LYPLA3, LYPLAL1, LYRM7, LYSMD3, LYSMD4, LYST, LZTFL1, M74720, MAB21L1, MACF1, MAD2L1, MADCAM1, MAFB, MAFF, MAFG, MAG1, MAGED2, MAGED4, MAGI1, MAGI2, MAGIX, Magmas, MAK3, MALL, MALT1, MAML2, MAML3, MAN1A2, MAN1B1, MAN1C1, MAN2A1, MAP1LC3C, MAP2K3, MAP2K5, MAP2K6, MAP2K7, MAP3K1, MAP3K2, MAP3K4, MAP3K5, MAP3K7, MAP4K3, MAP4K4, MAP6, MAP7D1, MAP7D3, MAPS, MAPK12, MAPK13, MAPK14, MAPK8, MAPKBP1, MARCKS, MARCKSL1, MARS, MASP1, MASP2, MAST4, MASTL, MATN2, MATR3, MBD3, MBD5, MBNL1, MBNL2, MBOAT2, MBP, MC5R, MCAM, MCART1, MCART6, MCC, MCM10, MCM2, MCM3, MCM3APAS, MCM4, MCM5, MCM6, MCM7, MCM8, MCOLN1, MCTP2, MDFI, MDFIC, MDM2, MDM4, MDN1, ME3, MECP2, MED13, MED13L, MED14, MED18, MED2O, MED26, MEF2B, MEG3, MEG8, MEGF8, MEGF9, MEI1, MEIS1, MEIS2, MEIS3, MEIS3P1, MELK, MEOX2, MESDC1, MEST, MET, METTL1, METTL3, METTLE, METTL7A, MEX3B, MEX3D, MFAP3L, MFAP4, MFSD2, MFSD7, MGA, MGC102966, MGC11102, MGC12916, MGC12935, MGC12965, MGC16121, MGC16169, MGC17403, MGC21874, MGC23270, MGC23985, MGC24103, MGC29891, MGC3260, MGC34034, MGC34646, MGC39584, MGC39900, MGC52110, MGC5370, MGC5566, MGC87042, MGLL, MIA3, MIAT, MIB1, MICAL2, MICALL1, MICB, MID1, MIER1, MINA, MIS12, MKI67, MKKS, MKL2, MKLN1, MKX, MLF1, MLF1IP, MLH3, MLL, MLL5, MLLT10, MLLT11, MLLT3, MLLT6, MLPH, MMD, MME, MMPI, MMP10, MMP14, MMP27, MN1, MND1, MON1A, MON1B, MOSC2, MOSPD2, MOSPD3, MOXD1, MPDZ, MPHOSPH1, MPHOSPH9, MPP4, MPPED2, MPZL1, MRAS, MRCL3, MREG, MRPL14, MRPL45, MRPL52, MRPS10, MRPS11, MRPS12, MRPS14, MRPS25, MRPS30, MRTO4, MSH4, MSI2, MSN, MSR1, MST150, MSTO1, MSTP9, MT1A, MT1F, MT1G, MT1H, MT1JP, MT1M, MT1X, MTA3, MTAP, MTBP, MTCH1, MTDH, MTF2, MTHFD1, MTHFD1L, MTHFD2L, MTHFR, MTL5, MTMR11, MTMR3, MTP18, MTRF1, MTRR, MTSS1, MTX3, MUC12, MUM1, MUPCDH, MVK, MX2, MXD1, MXD4, MXRA5, MYADM, MYBBP1A, MYBL2, MYC, MYCBP2, MYCL1, MYEOV, MYH10, MYH11, MYH8, MYL]P, MYLK, MYNN, MYO10, MYO15B, MYO19, MYO1B, MYO1D, MYO1E, MYO9B, MYST3, MYST4, N4BP2, NAALADL1, NAB2, NACA, NADK, NADSYN1, NAG13, NAGS, NAIP, NANOS1, NAP1L4, NAPB, NAPE-PLD, NAT10, NAV1, NAV2, NAV3, NBEA, NBL1, NBLA00301, NBPF1, NBPF10, NBPF3, NBR2, NCALD, NCAPD3, NCAPG, NCAPG2, NCAPH, NCAPH2, NCF2, NCOA1, NCOA3, NCR2, NDC80, NDE1, NDFIP2, NDP, NDUFA5, NDUFB2, NDUFC1, NEDD4L, NEDD9, NEFM, NEGR1, NEIL1, NEK1, NEK11, NEK2, NEK9, NELF, NETT, NETO2, NEU1, NEXN, NF1, NF2, NFAT5, NFATC2, NFATC21P, NFATC3, NFATC4, NFE2L2, NFIA, NFIB, NFIL3, NFIX, NFKB, NFKBIA, NFKBIB, NFKBIE, NFS1, NFYA, NFYB, NGLY1, NHEDC2, NHLRC2, NICN1, NIN, NINE, NIP7, NIPA2, NIPBL, NIPSNAP1, NIPSNAP3B, NISCH, NIT1, NKD2, NKPD1, NKX3-1, NLGN1, NLN, NLRC5, NME1, NME5, NMNAT2, NMT2, NNMT, NNT, NOC2L, NOC3L, NOC4L, NOG, NOL1, NOL12, NOL14, NOL3, NOL5A, NOL6, NOLC1, NOPE, NOS1, NOS2, NOS3, NOTCH2NL, NOV, NOVA1, NOX4, NP, N-PAC, NPAL2, NPAL3, NPAS2, NPB, NPC1, NPEPL1, NPEPPS, NPFFR1, NPHP1, NPHP3, NPL, NPR3, NPTX2, NR1D1, NR1D2, NR2Cl, NR2F2, NR3Cl, NR4A1, NR4A2, NR6A1, NRBP2, NRG1, NRIP1, NRIP3, NRP1, NRP2, NSBP1, NSMCE4A, NSUN2, NSUN6, NT5DC1, NT5E, NTN4, NTRK3, NUBPL, NUDCD1, NUDT10, NUDT13, NUDT14, NUDT15, NUDT16, NUDT3, NUDT6, NUDT7, NUF2, NDFIP2, NUMA1, NUMB, NUP107, NUP155, NUP35, NUP50, NUP62, NUP85, NUP93, NUP98, NUPL1, NUSAP1, NXF1, NXT1, NY-SAR-48, OAZ2, OAZ3, OBFC2A, OBSCN, OBSL1, ODC1, ODZ3, OGDH, OGFOD2, OGN, OGT, OIP5, OLFML1, OLFML2A, OLFML2B, OMA1, OMD, OPA1, OPA3, OPCML, OPLAH, OPRD1, OPRL1, OPTN, OR10A5, ORAI1, ORAOV1, ORC1L, ORC6L, ORMDL1, OSAP, OSBPL10, OSBPL1A, OSBPL2, OSBPL3, OSBPL6, OSBPL7, OSGIN1, OSGIN2, OSMR, OSR1, OSR2, OSTM1, OTUD6A, OTUD7A, OTUD7B, Pl8SRP, P2RX5, P2RY4, P2RY5, P4HA3, PA2G4, PABPC1, PABPC5, PABPN1, PACS2, PACSIN1, PACSIN3, PAF1, PAFAH1B1, PAFAH1B3, PAFAH2, PAG1, PAK1, PALB2, PALLD, PANS, PANK2, PAPD1, PAPOLA, PAPOLG, PAPPA, PAPPA2, PAQR3, PAQR4, PARD3, PARD3B, PARP1, PARP14, PARP2, PARP3, PARP4, PARP9, PATZ1, PAWR, PAXIP1, PBEF1, PBK, PBLD, PBX1, PBXIP1, PCAF, PCBD1, PCDH18, PCDH7, PCDH9, PCDHB16, PCF11, PCGF5, PCM1, PCMTD1, PCMTD2, PCNA, PCNX, PCNXL2, PCOTH, PCSK1, PCSK9, PCTK2, PCYOX1, PDCD11, PDCD1LG2, PDCD4, PDCD61P, PDE11A, PDE1A, PDE4A, PDE4B, PDE4DIP, PDE5A, PDE6D, PDE7B, PDGFD, PDGFRA, PDGFRB, PDGFRL, PDK2, PDK4, PDLIM4, PDLIM5, PDLIM7, PDPR, PDRG1, PDS5B, PDSS1, PDXK, PDXP, PDZRN3, PEAR1, PECR, PELI1, PELI2, PEO1, PER1, PER2, PER3, PERLD1, PEX13, PF4, PF4V1, PFAAP5, PFDN2, PFKFB2, PFKFB3, PFKP, PFN2, PGA3, PGAM5, PGAP1, PGBD3, PGCP, PGF, PGGT1B, PGM2L1, PGPEP1, PGRMC1, PGRMC2, PGS1, PHACS, PHACTR2, PHACTR3, PHCl, PHC2, PHC3, PHEX, PHF10, PHF17, PHF19, PHF2, PHF20L1, PHGDH, PHIP, PHKB, PHKG1, PHLDA1, PHLDA2, PHTF2, PHYHD1, PIAS2, PIAS3, PICALM, PICK1, PID1, PIFI1, PIGB, PIGG, PIGH, PIGK, PIGL, PIGN, PIGV, PIGW, PIH1D2, PIK3C2A, PIK3CA, PIK3CD, PIK31P1, PIK3R1, PIK3R3, PINX1, PIP5K3, PIR, PITPNC1, PITX2, PKD2, PKIA, PKMYT1, PKNOX1, PKP4, PLA2G12A, PLA2G3, PLA2R1, PLAC1, PLAC2, PLAC7, PLAG1, PLAT, PLAU, PLAUR, PLCB1, PLCD3, PLCL2, PLCXD1, PLD1, PLEC1, PLEK2, PLEKHA2, PLEKHA5, PLEKHA8, PLEKHA9, PLEKHG5, PLEKHH2, PLEKHK1, PLEKHO1, PLGLB2, PLK1, PLK3, PLK4, PLOD2, PLSCR4, PLXNA1, PLXNA2, PMAIP1, PML, PMVK, PNO1, PNPLA4, PODXL, POGZ, POLA1, POLA2, POLD1, POLE, POLE2, POLE3, POLH, POLI, POLK, POLQ, POLR2D, POLR2H, POLR3H, POLR3K, POLRMT, POPS, POP7, POPDC3, POT1, PPA2, PPAN, PPAP2A, PPAP2B, PPAPDC1A, PPAPDC3, PPARA, PPARGC1A, PPARG, PPAT, PPCDC, PPFIBP1, PPFIBP2, PPHLN1, PPID, PPIF, PPIG, PPIH, PPIL1, PPIL2, PPIL5, PPL, PPM1A, PPM1D, PPM1G, PPM1M, PPME1, PPDX, PPP1CB, PPP1R10, PPP1R13L, PPP1R14A, PPP1R14c, PPP1R15A, PPP1R3C, PPP2R2D, PPP2R3A, PPP2R3B, PPP2R5E, PPP3CA, PPP3R1, PPP4R2, PPRC1, PPTC7, PQLC2, PQLC3, PRAGMIN, PRC1, PRDM1, PRDM2, PRDX3, PRELID1, PREPL, PRICKLE1, PRIM1, PRKAA1, PRKAR1A, PRKCA, PRKCE, PRKCSH, PRKD1, PRKD3, PRKG1, PRKRA, PRLR, PRMT2, PRO0149, PRO1051, PRO1146, PRO1843, PRO2158, PRO2221, PRO2550, PRO3098, PRO3121, PRO51, PRPF4, PRPH, PRPS1, PRR11, PRR14, PRR15, PRR16, PRR3, PRR6, PRR7, PRRT1, PRRT2, PRRX1, PRRX2, PRSS1, PRSS12, PRSS2, PRSS23, PRSS3, PRTFDC1, PRUNE2, PS1TP4, PSAP, PSD3, PSG1, PSG2, PSG4, PSG9, PSMB7, PSMB9, PSMC3, PSMC31P, PSMD10, PSMD12, PSMD2, PSMD3, PSME3, PSME4, PSPH, PSRC1, PTAR1, PTBP1, PTCD1, PTCD3, PTDSS2, PTEN, PTGER2, PTGER3, PTGES, PTGFR, PTGFRN, PTGIR, PTGIS, PTGS2, PTHLH, PTP4A1, PTPLAD2, PTPN11, PTPN13, PTPN21, PTPN22, PTPRB, PTPRE, PTPRF, PTPRM, PTPRO, PTPRR, PTPRS, PTPRU, PTRH2, PTTG1, PTTG3, PTX3, PUS1, PUS7, PUSL1, PVR, PVRL2, PWP2, PXMP3, PXMP4, PXN, PYCARD, PYGL, PYGM, PYROXD1, QKI, QPRT, QSOX1, QSOX2, R3HDM2, RAB11A, RAB18, RAB23, RAB26, RAB27A, RAB27B, RAB30, RAB35, RAB38, RAB3D, RAB3GAP2, RAB42, RAB7, RAB7A, RAB7B, RAB8B, RAB9B, RABGAP1, RABGEF1, RABIF, RACGAP1, RAD18, RAD21, RAD23B, RAD51, RAD51AP1, RAD51L3, RAD52, RAD54B, RAD54L, RAD9A, RAGE, RAI1, RALA, RALBP1, RALGPS2, RANBP1, RANBP9, RANGAP1, RAP2B, RAP2C, RAPGEF1, RAPGEF6, RAPH1, RARA, RASA3, RASAL2, RASD1, RASD2, RASEF, RASGEF1A, RASL10B, RASL11B, RASSF2, RASSF4, RAVER2, RB1CC1, RBBP6, RBCK1, RBJ, RBL2, RBM12, RBM14, RBM17, RBM23, RBM24, RBM30, RBM33, RBM35B, RBM41, RBM43, RBM4B, RBM6, RBM7, RBM9, RBMS3, RBPMS, RC3H1, RC3H2, RCAN1, RCAN2, RCL1, RCN3, RCP9, RECK, RECQL4, REEP3, REEP4, RELB, RELT, RETN, REV1, REV3L, RFC2, RFC3, RFC4, RFC5, RFK, RFTN1, RFTN2, RFWD3, RFX3, RGN, RGS12, RGS16, RGS17, RGS2, RGS20, RGS3, RHBDF2, RHEB, RHEBL1, RHOB, RHOBTB1, RHOBTB3, RHOJ, RHOQ, RICS, RICTOR, RIFT, RIG, RIN2, RIN3, R10K3, RIPK3, RIPK4, RIPK5, RITZ, RLTPR, RNASEH2A, RNASEL, RND1, RND3, RNFLJ1, RNF12, RNF122, RNF126, RNF128, RNF144B, RNF149, RNF150, RNF157, RNF170, RNF207, RNF34, RNF5, RNMTL1, ROBOT, ROBO2, ROCK2, ROGDI, ROPN1L, ROR1, ROR2, RORA, RP11-125A7.3, RP11-298P3.3, RP1-21018.1, RP13-15M17.2, RP3-473B4.1, RP5-1022P6.2, RP5-886K2.1, RPA2, RPL13, RPL21, RPL31, RPL35, RPL37A, RPL39L, RPP25, RPPH1, RPS15A, RPS24, RPS6KA2, RQCD1, RRAD, RRAGB, RRAGD, RRBP1, RREB1, RRM2, RRN3, RRP1, RRP12, RRP15, RRP9, RRS1, RSHL2, RSL1D1, RSRC1, RTN2, RTP4, RUNX1, RUNX1T1, RUTBC1, RWDD2B, RXFP3, RXRA, RYBP, S100A10, S100A2, S100A6, S100A7, S100P, SAC3D1, SACS, SAFB2, SALL1, SALL2, SAMD1, SAMD11, SAMD4A, SAMD9L, SAMHD1, SAP30, SAPS2, SARM1, SASH1, SAT, SAT1, SATB1, SATB2, SATL1, SAV1, SBF2, SC4MOL, SC5DL, SCCPDH, SCD, SCFV, SCG5, SCLT1, SCLY, SCRG1, SCRN3, SCUBE3, SCYE1, SDC2, SDC3, SDCBP2, SDCCAG3, SDCCAG8, SDF2L1, SDHAL2, SDHALP2, SDHC, SDPR, SEC11C, SEC14L1, SEC14L2, SEC22C, SEC23B, SEC24A, SEC24C, SEC31L1, SECTM1, SEH1L, SEL1L, SELENBP1, SELI, SELO, SELPLG, SEMA3A, SEMA3B, SEMA3C, SEMA3D, SEMA4A, SEMA4C, SEMA5A, SEMA6D, SENP5, SENP6, SENP7, SENP8, SEPHS1, SEPP1, SEPSECS, SERAC1, SERGEF, SERHL, SERHL2, SERPINB1, SERPINB2, SERPINB6, SERPINB7, SERPINB8, SERPINE1, SERPINE2, SERPINF1, SERTAD1, SERTAD4, SESN3, SETBP1, SETDB2, SF3B1, SF3B2, SFN, SFPQ, SFRP2, SFRP4, SFRS1, SFRS11, SFRS12, SFRS15, SFRS18, SFRS21P, SFRS3, SFRS5, SFRS6, SFRS7, SFXN3, SFXN5, SGCD, SGCE, SGCG, SGK, SGK269, SGMS2, SGOL2, SGSH, SGSM2, SGTA, SH2B3, SH2D2A, SH3BGRL, SH3BP2, SH3BP4, SH3BP5, SH3D19, SH3GLB1, SH3GLB2, SH3GLP3, SH3MD4, SH3RF1, SHCl, SHC2, SHC4, SHCBP1, SHOX2, SHPRH, SHROOM3, SIAE, SIGIRR, SIKE, SIM1, SIMP, SIPA1L1, SIPA1L2, SIPA1L3, SIRPA, SIRT1, SIRT2, SIRT3, SIRT4, SIRT7, SIX4, SIX5, SKAP2, SKI, SKP2, SLA/LP, SLAIN2, SLC12A2, SLC12A4, SLC14A1, SLC15A3, SLC15A4, SLC16A1, SLC16A12, SLC16A14, SLC16A3, SLC16A6, SLC16A7, SLC19A1, SLC19A2, SLC1A2, SLC1A3, SLC1A4, SLC20A1, SLC20A2, SLC22A4, SLC24A1, SLC25A13, SLC25A22, SLC25A_{—}23, SLC25A27, SLC25A28, SLC25A33, SLC25A36, SLC25A37, SLC25A44, SLC27A3, SLC27A4, SLC29A1, SLC2A1, SLC2A10, SLC2A14, SLC2A3, SLC2A6, SLC30A1, SLC30A9, SLC31A2, SLC35A2, SLC35A3, SLC35B1, SLC35C2, SLC35E2, SLC35E4, SLC35F2, SLC36A1, SLC38A1, SLC38A4, SLC38A5, SLC39A11, SLC3A2, SLC43A2, SLC44A1, SLC46A3, SLC4A2, SLC4A4, SLC4A7, SLC4A8, SLC5A3, SLC5A6, SLC6A19, SLC6A6, SLC6A8, SLC7A1, SLC7A11, SLC7A5, SLC7A6, SLC8A1, SLC9A3, SLC9A5, SLC9A9, SLFN11, SLIT2, SLITS, SLK, SLN, SLTM, SMAD2, SMAD3, SMAD4, SMAD5, SMAD7, SMARCA1, SMARCA2, SMARCA4, SMARCB1, SMARCC2, SMARCD2, SMC2, SMC4, SMCHD1, SMCR7L, SMG6, SMOX, SMPD4, SMTN, SMURF2, SMYD3, SMYD4, SNAG1, SNAIL SNAP23, SND1, SNED1, SNF1LK2, SNHG10, SNHG5, SNHG7, SNHG9, SNORA28, SNORD114-3, SNRP70, SNRPA1, SNRPG, SNRPN, SNTB2, SNX1, SNX11, SNX12, SNX13, SNX17, SNX5, SNX7, SNX8, SOAT1, SOCS1, SOCS3, SOCS5, SOCS7, SOD1, SOD2, SOLH, SORBS2, SORD, SOS1, SOST, SOX4, SOX9, SP110, SPA17, SPAG16, SPAG5, SPANXA2, SPAST, SPATA13, SPATA17, SPATA18, SPATA20, SPATA6, SPATA7, SPC24, SPC25, SPCS3, SPEF2, SPG11, SPG3A, SPG7, SPHK1, SPIN2B, SPINS, SPINK1, SPNS1, SPOCD1, SPP1, SPRED1, SPRY1, SPRY2, SPRY4, SPSB1, SPTBN1, SPTLC2, SPTY2D1, SQLE, SQSTM1, SR140, SRGAP2P1, SRGN, SRM, SRPK2, SRPRB, SRXN1, SS18, SSBP2, SSH1, SSPN, SSR3, SSSCA1, ST3GAL1, ST6GALNAC2, ST6GALNAC5, ST6GALNAC6, ST7L, ST8SIA1, STAC, STAM2, STAMBPL1, STARD13, STARD3, STARD4, STARD5, STARD8, STAT2, STATS, STAT4, STATIP1, STC1, STC2, STEAP1, STIL, STIP1, STK10, STK1HP, STK17A, STK17B, STK32B, STK32C, STK36, STK38, STK4, STMN1, STOML1, STON1, STRA13, STRA6, STS-1, STX17, STX1A, STX3, STX6, STXBP5, STXBP6, STYX, SUDS3, SUGT1L1, SUOX, SUPT4H1, SURF-4, SUV39H1, SUV420H1, SUZ12P, SVEP1, SWAP70, SYDE2, SYNCRIP, SYNE1, SYNGR1, SYNGR2, SYNJ2, SYNJ2BP, SYNPO2, SYT11, SYT15, SYTL2, SYTL3, SYTL4, T62549, T70285, TAC1, TACC3, TAF13, TAF1B, TAF3, TAF4, TAF4B, TAF6L, TAGLN, TAGLN3, TAP1, TAP2, TAPBPL, TAS2R44, TAT, TATDN2, TAX1BP1, TBC1D12, TBC1D16, TBC1D17, TBC1D2, TBC1D24, TBC1D2B, TBC1D3F, TBC1D5, TBC1D8, TBC1D8B, TBCA, TBCD, TBL1XR1, TBRG1, TBX15, TBX18, TBX2, TBX3, TBX5, TCEA1, TCEA2, TCEA3, TCEB3, TCF12, TCF15, TCF19, TCF25, TCF3, TCF4, TCF7L1, TCF7L2, TCF8, TCIRG1, TCTEX1D1, TDG, TDO2, TDP1, TEAD1, TEAD4, TEF, TELO2, TENC1, TEP1, TERF2, TERT, TES, TESK1, TEX10, TFDP1, TFDP2, TFPI2, TGFA, TGFB1, TGFBR3, TGM2, TH, THADA, THAP2, THAP5, THBD, THBS1, THBS2, THC2235542, THC2266906, THC2274697, THC2278725, THC2279825, THC2279910, THC2280343, THC2280741, THC2281350, THC2282972, THC2284350, THC2290002, THC2308340, THC2311764, THC2312756, THC2312785, THC2312955, THC2314215, THC2315330, THC2316649, THC2316768, THC2316936, THC2317182, THC2319152, THC2320257, THC2322443, THC2324430, THC2337372, THC2337493, THC2338537, THC2339241, THC2339455, THC2340757, THC2342473, THC2343350, THC2345075, THC2356023, THC2358845, THC2360810, THC2360912, THC2361491, THC2361914, THC2368209, THC2369020, THC2374442, THC2375512, THC2375853, THC2376418, THC2376586, THC2378378, THC2378839, THC2378865, THC2378994, THC2381061, THC2381707, THC2382717, THC2397757, THC2400593, THC2404671, THC2405319, THC2405710, THC2405842, THC2405936, THC2406017, THC2406779, THC2406786, THC2406944, THC2407334, THC2407737, THC2408033, THC2408757, THC2408828, THC2409451, THC2411515, THC2419011, THC2437177, THC2439773, THC2440027, THC2441367, THC2448178, THC2449905, THC2453866, THC2455353, THEM4, THOC4, THOC6, THOP1, THRAP2, THRAP3, THRB, THSD1, THSD4, THYN1, TIAL, TIAM2, TIGD1L, TIGD3, TIMELESS, TIMM10, TIMM13, TIMM22, TIMM50, TIMM8A, TIMP3, TIMP4, TINF2, TIPIN, TJP2, TK2, TLCD1, TLE3, TLE4, TLK1, TLN1, TLN2, TLOC1, TM2D1, TM4SF1, TM4SF4, TM7SF3, TMBIM1, TMC8, TMCC1, TMCO3, TMCO7, TMED4, TMEM100, TMEM103, TMEM106B, TMEM107, TMEM109, TMEM110, TMEM112, TMEM117, TMEM119, TMEM132D, TMEM140, TMEM150, TMEM154, TMEM158, TMEM162, TMEM166, TMEM168, TMEM170, TMEM171, TMEM19, TMEM22, TMEM29, TMEM30A, TMEM30B, TMEM33, TMEM35, TMEM37, TMEM38B, TMEM39A, TMEM42, TMEM46, TMEM47, TMEM48, TMEM57, TMEM63A, TMEM67, TMEM81, TMEPAI, TMF1, TMLHE, TMPO, TMTC1, TMTC2, TMTC3, TMTC4, TNC, TncRNA, TNFAIP3, TNFAIP6, TNFAIP8L1, TNFRSF10A, TNFRSF10B, TNFRSF10D, TNFRSF11B, TNFRSF12A, TNFRSF19, TNFRSF1B, TNFRSF21, TNFRSF25, TNFRSF6B, TNFSF12, TNFSF4, TNFSF9, TNIP2, TNKS, TNKS1BP1, TNNC1, TNNC2, TNRC15, TNRC4, TNRC6A, TNRC6B, TNRC6C, TNRC8, TNS3, TNXB, TOB1, TOE1, TOLLIP, TOM1, TOMM34, TOMM40, TOP1, TOP2A, TOX, TOX2, TP53, TP53AP1, TP531NP1, TP531NP2, TPARL, TPCN1, TPCN2, TPD52L1, TPI1, TPM1, TPM2, TPM4, TPP1, TPR, TPX2, TRA16, TRA2A, TRABD, TRAF3, TRAF31P1, TRAF31P2, TRAF5, TRAFD1, TRAIP, TRAK1, TRAM2, TRAPPC2, TRAPPC6A, TRERF1, TRIB1, TRIB2, TRIM13, TRIM16, TRIM2, TRIM21, TRIM22, TRIM23, TRIM33, TRIM4, TRIM44, TRIM45, TRIMS, TRIM56, TRIM59, TRIM69, TRIM73, TR10, TRIOBP, TRIP11, TRIP12, TRIP13, TRMT1, TRMT6, TRO, TROAP, TROVE2, TRPC1, TRPM7, TRPS1, TRPV2, TRY6, TSC22D3, TSC22D4, TSEN54, TSGA10, TSHZ1, TSHZ2, TSHZ3, TSPAN13, TSPAN14, TSPAN18, TSPAN2, TSPAN9, TSR1, TSR2, TSTA3, TTBK2, TTC12, TTC28, TTC3, TTC30B, TTC32, TTK, TTL, TTLL12, TTLL3, TTRAP, TTTY14, TUB, TUBA4A, TUBB2A, TUBB3, TUBB4, TUBG1, TUG1, TUSC3, TWIST2, TXLNA, TXN2, TXNDC4, TXNIP, TXNL4B, TXNRD1, TYRO3, U87972, UACA, UAP1, UAP1L1, UBE1L, UBE1L2, UBE2C, UBE2E1, UBE2H, UBE2M, UBE2NL, UBE2S, UBE2T, UBE2V2, UBE4B, UBIAD1, UBL3, UBN1, UBQLN1, UBQLN4, UBQLNL, UBR2, UBR4, UBXD1, UBXD5, UBXD7, UCHL5, UCK2, UEV3, UGCG, UHRF1, ULBP2, ULK2, UNC119, UNC84A, UNC84B, UNG, UNKL, UNQ338, UPP1, URLC9, USF2, USP10, USP21, USP25, USP3, USP30, USP32, USP34, USP36, USP45, USP47, USP52, USP53, USP54, USP6NL, USP7, USP9X, UST, UTP15, UTS2D, VAC14, VAMP4, VAPA, VAPB, VARS, VASH1, VASP, VCAN, VCP, VCPIP1, VDP, VDR, VEGFA, VEGFC, VEPH1, VGLL3, VIL2, VIM, VISA, VIT, VMD2, VPRBP, VPS13A, VPS13B, VPS13C, VPS13D, VPS24, VPS36, VPS41, VPS4A, VPS53, VRK1, VSIG8, VTI1A, WO5707, WAPAL, WASF2, WBP1, WDFY2, WDFY3, WDHD1, WDR13, WDR19, WDR31, WDR32, WDR4, WDR45, WDR5, WDR51A, WDR60, WDR68, WDR76, WDR77, WDR79, WDR82, WFDC1, WFDC3, WFS1, WHSC1L1, WIPF1, WIPF2, WISP1, WISP2, WNK1, WNK4, WNT10A, WNT11, WNT16, WNT2, WNT5A, WNT5B, WRNIP1, WSB1, WSB2, WTAP, WWC1, WWC2, WWOX, WWTR1, XAF1, XDH, XG, XIST, XPA, XPNPEP3, XPO1, XPO5, XPOT, XRCC4, XRCC6BP1, XRN1, XRRA1, XYLT1, YAP1, YIPF6, YKT6, YOD1, YPEL1, YPEL2, YPEL3, YPEL4, YRDC, YTHDC2, YTHDF3, Z28739, ZAK, ZBED1, ZBED3, ZBED5, ZBTB10, ZBTB20, ZBTB24, ZBTB26, ZBTB3, ZBTB34, ZBTB41, ZBTB43, ZBTB44, ZC3H13, ZC3H6, ZC3HAV1L, ZCCHC10, ZCCHC11, ZCCHC3, ZDBF2, ZDHHC14, ZDHHC17, ZDHHC2, ZDHHC21, ZDHHC22, ZDHHC3, ZDHHC5, ZEB1, ZEB2, ZFAND2A, ZFAND2B, ZFAND5, ZFAND6, ZFHX3, ZFHX4, ZFP1, ZFP106, ZFP3, ZFP36L2, ZFP90, ZFPL1, ZFPM2, ZFX, ZFY, ZFYVE16, ZGPAT, ZHX1, ZHX2, ZHX3, ZIK1, ZKSCAN1, ZMAT3, ZMIZ1, ZMYM2, ZMYM4, ZMYM5, ZMYND10, ZMYND11, ZNF101, ZNF107, ZNFLJ7, ZNF12, ZNF131, ZNF14, ZNF148, ZNF160, ZNF165, ZNF167, ZNF174, ZNF182, ZNF189, ZNF19, ZNF192, ZNF20, ZNF207, ZNF217, ZNF223, ZNF224, ZNF225, ZNF226, ZNF228, ZNF230, ZNF232, ZNF236, ZNF24, ZNF248, ZNF252, ZNF253, ZNF259, ZNF264, ZNF267, ZNF273, ZNF277, ZNF280D, ZNF282, ZNF286A, ZNF289, ZNF292, ZNF294, ZNF297B, ZNF302, ZNF313, ZNF317, ZNF323, ZNF326, ZNF331, ZNF333, ZNF334, ZNF33A, ZNF345, ZNF347, ZNF350, ZNF354C, ZNF367, ZNF37B, ZNF395, ZNF397, ZNF404, ZNF408, ZNF415, ZNF420, ZNF423, ZNF43, ZNF430, ZNF432, ZNF438, ZNF439, ZNF440, ZNF441, ZNF446, ZNF449, ZNF462, ZNF468, ZNF473, ZNF512, ZNF512B, ZNF516, ZNF517, ZNF521, ZNF529, ZNF532, ZNF551, ZNF555, ZNF557, ZNF560, ZNF564, ZNF567, ZNF569, ZNF57, ZNF573, ZNF585A, ZNF587, ZNF597, ZNF599, ZNF605, ZNF606, ZNF615, ZNF618, ZNF621, ZNF622, ZNF623, ZNF627, ZNF630, ZNF652, ZNF655, ZNF658, ZNF662, ZNF672, ZNF675, ZNF677, ZNF680, ZNF681, ZNF684, ZNF688, ZNF691, ZNF697, ZNF70, ZNF702, ZNF708, ZNF709, ZNF710, ZNF713, ZNF717, ZNF75, ZNF768, ZNF770, ZNF783, ZNF785, ZNF792, ZNF805, ZNF81, ZNF814, ZNF818, ZNF84, ZNF85, ZNF91, ZNF92, ZNF93, ZRF1, ZSCAN29, ZSWIM6, ZWILCH, ZWINT, ZXDB, ZYG11B, and ZYX.

It is also recognized that the change in expression is directional, in that for some genes it is beneficial to increase expression in order to enhance (or reduce) longevity, health, or biological wellbeing—while the expression of other genes needs to be decreased for the same purpose. For instance, with regard to telomeres, it is beneficial (for maintenance of the telomere and therefore increased longevity/health/wellbeing) to upregulate (increase the expression of) TNKS and POT1, while it is beneficial to downregulated (decrease the expression of) TNKS2, TRF1, TIN2, and/or TRF2. Likewise, the following four tables provide beneficial up- and down-regulation indications for genes found on two specific arrays provided herein (Array 1 and Array 2), as well as the genes involved in mitochondrial maintenance and DNA repair. In each table, the shaded genes are beneficially downregulated for longevity/health/etc., while the unshaded genes are beneficially upregulated. Methods and compositions are provided herein that can accomplish up- and down-regulation of these genes.

Array #1

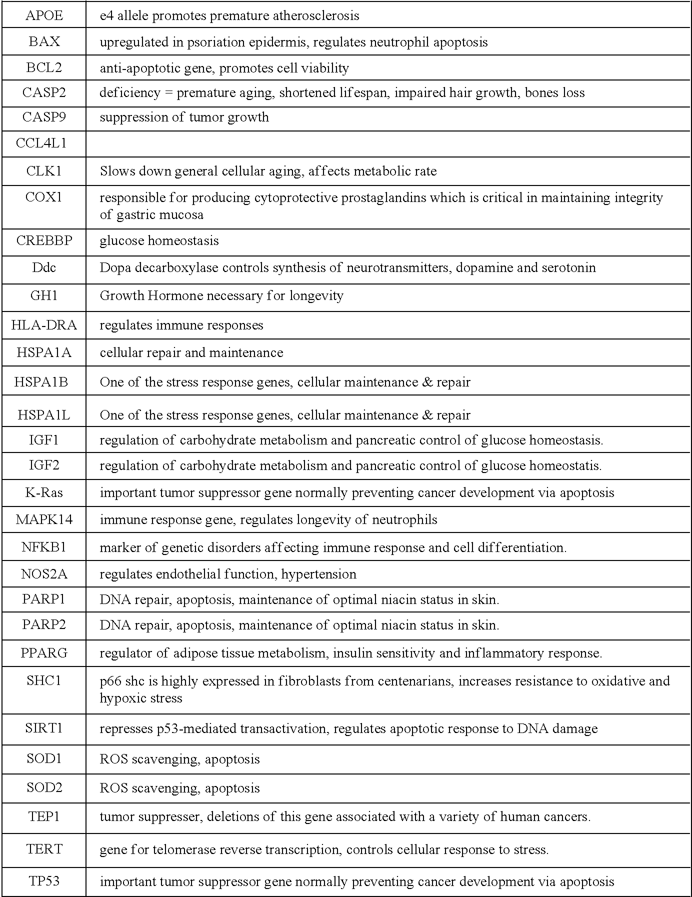

Array 2.0

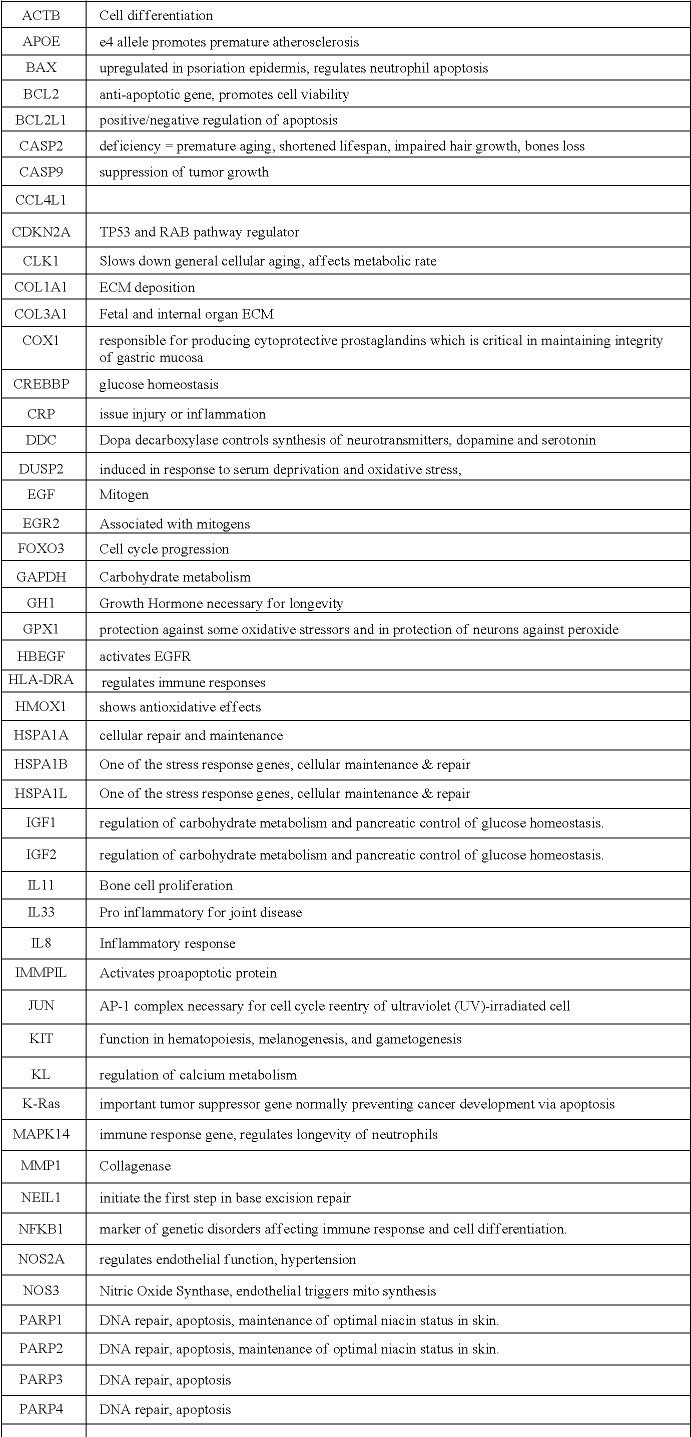

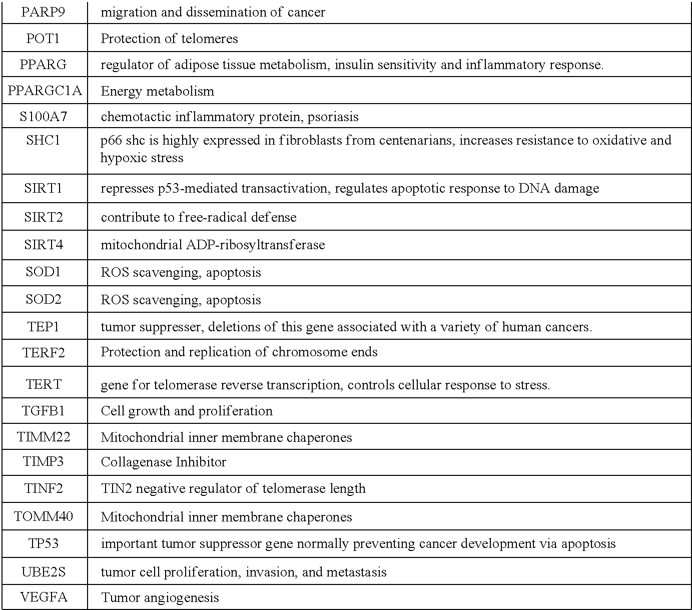

Mitochondria biogenesis, maintenance, etc.

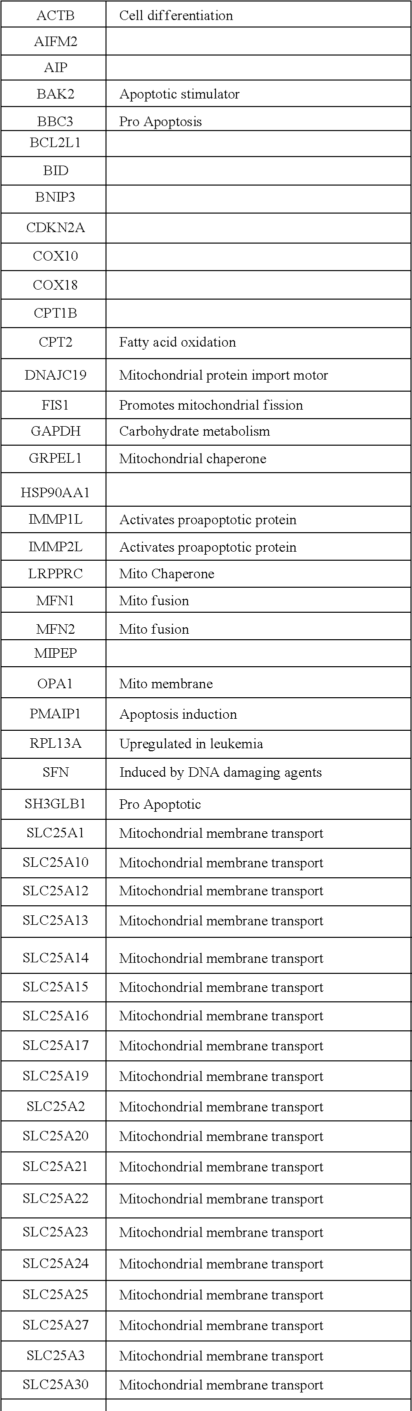

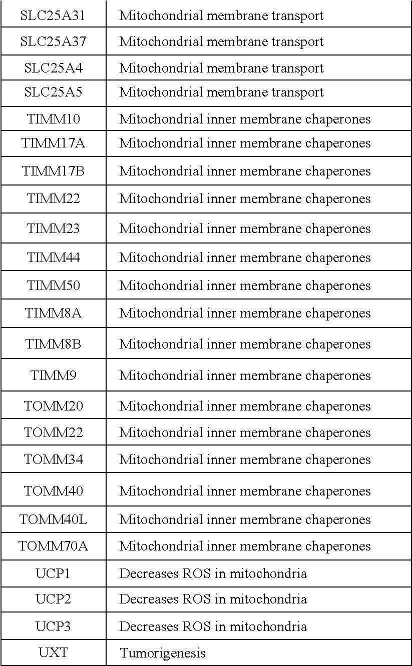

DNA Repair, maintenance, etc.

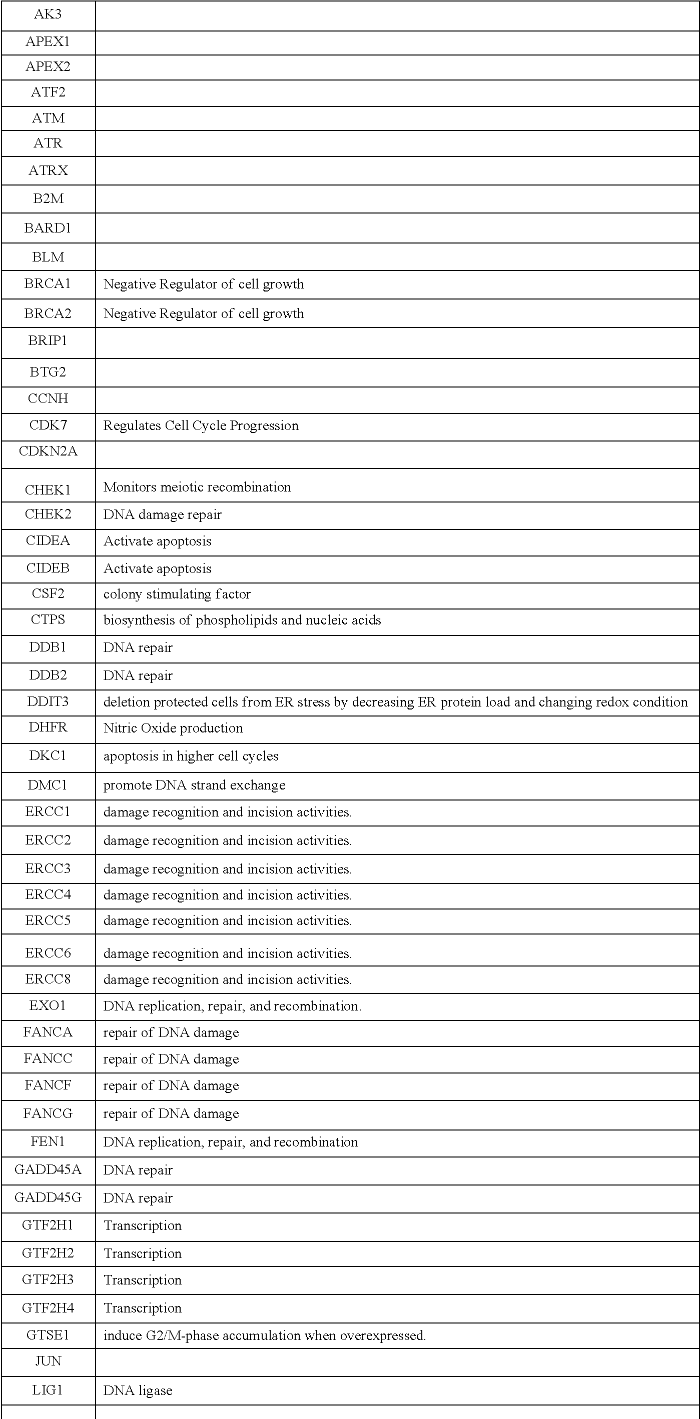

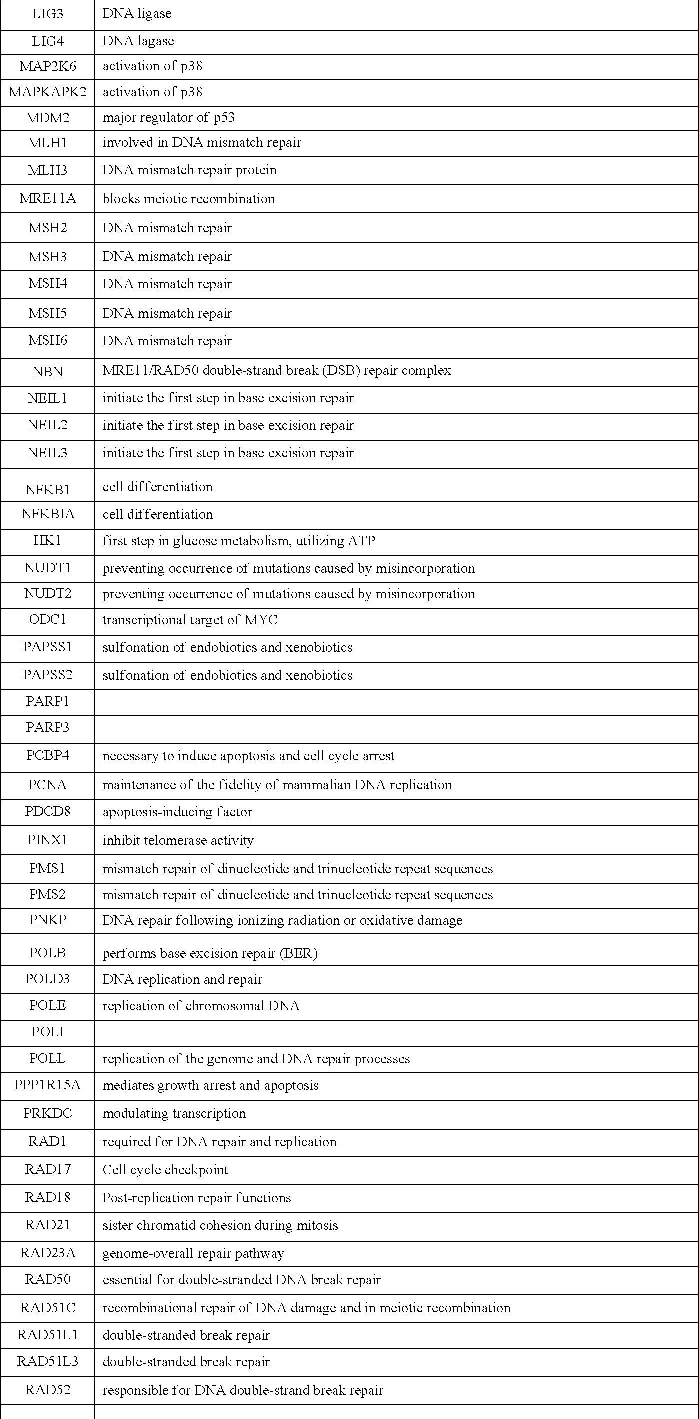

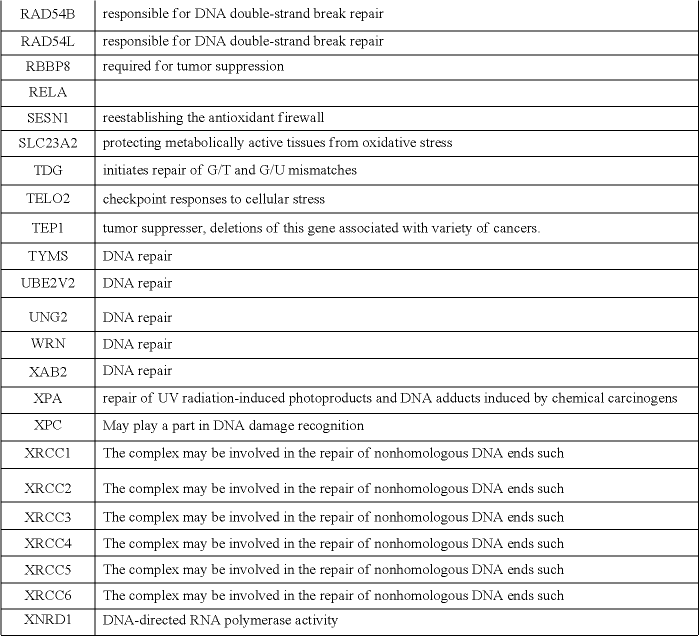

Additional contemplated sets of lifespan or longevity responsive genes include (without limitation): TERT, TERC, NRF2, POT1, TRF1, TRF2, TIN2, TPP1, RAPT, TNKS, and TNKS 2; TERF2, TERF2IP, POLG, POLB, POLD3, POLE, POLI, POLL, PARP2, PPARG, SHCl, PTOP, IFI44 and NFKB1; HSPA1A, HSPA1B, and HSPA1L; MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70; TERT, TERC, NRF2, PARP1, POT1, TRF1, TRF2, TIN2, TPP1, RAP1, Tankyrase 1, Tankyrase 2, TERF2, TERF2IP, POLG, POLB, POLD3, POLE, POLI, POLL, PARP2, PPARG, SHCl, PTOP, IFI44, NFKB1, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70; TERF2, TERF2IP, POLG, POLB, POLD3, POLE, POLI, POLL, PARP2, PPARG, SHCl, HSPA1A, HSPA1B, and HSPA1L; PARP1, POT1, TRF1, TRF2, TIN2, TPP1, RAP1, Tankyrase 1, Tankyrase 2, TERF2, TERF2IP, POLG, POLB, POLD3, POLE, POLI, POLL, PARP2, PPARG, SHCl, PTOP, IFI44, NFKB1, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL, KU70, HSPA1A, HSPA1B, and HSPA1L; TERT, TERC, NRF2, PARP1, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHCl, PTOP, IFI44, NFKB1, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL, KU70, HSPA1A, HSPA1B, and HSPA; PGC1α, SIRT1, SIRT3, SIRT4, SIRT5, NRF1 and Tfam; and TNKS, TNKS2, TRF1, TIN2, TPP1, POT1, RAP1, TRF2, and TERT. Also contemplated are subsets of any of these lists.

### Exemplary Methods and Compositions

Provided herein are various methods and compositions for modulating gene expression or protein production or cell signaling which controls the maintenance of the telomere and/or which controls the biogenesis or respiratory activity of mitochondria and/or which control the lifespan, rate of aging, senescence, onset of disease states, or response to stress including apoptosis and cell death for a living cell, tissue, organ or organism.

The methods comprise contacting at least one cell with a sufficient amount of a modulating compound, or combination of compounds either simultaneously exposed or sequentially exposed. These compositions include the described modulating agents as well as their analogs, derivatives from naturally occurring, biosynthetic or bioengineered sources. Exemplary routes of achieving contact with such modulating agent or agents may involve any known method of delivery or contact for at least one cell, tissue, organ or organism in vivo or ex vivo or in vitro.

It is believed that plants from any plant Division, including Bryophyta, Psilophyta, Lycophyta, Equisetophyta, Filicophyta, Coniferophyta, Ginkgophyta, Cycadophyta, Gnetophyta, and Angiospermophyta.

Without intending to be limited to compounds or compositions derived from particular plants, the following specific plants are contemplated for preparing lifespan influencing compositions: coffee (e.g., coffee cherry extract), green tea (e.g., green tea extract), blueberries (Alaskan, for instance), cranberries, huckleberries, acai berries, goji berries, blackberries, raspberries, grapes (scupernog), strawberries, persimmon, pomegranate, lingonberry, bearberry, mulberry, bilberry, choke cherry, sea buckthorn berries, goji berry, tart cherry, kiwi, plum, apricot, apple, banana, berry, blackberry, blueberry, cherry, cranberry, currant, greengage, grape, grapefruit, gooseberry, lemon, mandarin, melon, orange, pear, peach, pineapple, plum, raspberry, strawberry, sweet cherry, watermelon, and wild strawberry. In addition, extracts from trees and bushes are also contemplated, including for instance sequoia, coastal redwood, bristlecone pine, birch, cedar of Lebanon, frankincense, and so forth.

By way of additional examples, compositions may be from leafy or salad vegetables [e.g., Amaranth (Amaranthus cruentus), Arugula (Eruca sativa), Beet greens (Beta vulgaris subsp. vulgaris), Bitterleaf (Vernonia calvoana), Bok choy (Brassica rapa Chinensis group), Broccoli Rabe (Brassica rapa subsp. rapa), Brussels sprout (Brassica oleracea Gemmifera group), Cabbage (Brassica oleracea Capitata group), Catsear (Hypochaeris radicata), Celery (Apium graveolens), Celtuce (Lactuca sativa var. asparagina), Ceylon spinach (Basella alba), Chard (Beta vulgaris var. cicla), Chaya (Cnidoscolus aconitifolius subsp. aconitifolius), Chickweed (Stellaria), Chicory (Cichorium intybus), Chinese cabbage (Brassica rapa Pekinensis group), Chinese Mallow (Malva verticillata), Chrysanthemum leaves (Chrysanthemum coronarium), Collard greens (Brassica oleracea), Corn salad (Valerianella locusta), Cress (Lepidium sativum), Dandelion (Taraxacum officinale), Endive (Cichorium endivia), Epazote (Chenopodium ambrosioides), Fat hen (Chenopodium album), Fiddlehead (Pteridium aquilinum, Athyrium esculentum), Fluted pumpkin (Telfairia occidentalis), Garden Rocket (Eruca sativa), Golden samphire (Inula crithmoides), Good King Henry (Chenopodium bonus-henricus), Greater Plantain (Plantago major), Kai-lan (Brassica rapa Alboglabra group), Kale (Brassica oleracea Acephala group), Komatsuna (Brassica rapa Pervidis or Komatsuna group), Kuka (Adansonia spp.), Lagos bologi (Talinum fruticosum), Land cress (Barbarea verna), Lettuce (Lactuca sativa), Lizard's tail (Houttuynia cordata), Melokhia (Corchorus olitorius, Corchorus capsularis), Mizuna greens (Brassica rapa Nipposinica group), Mustard (Sinapis alba), New Zealand Spinach (Tetragonia tetragonioides), Orache (Atriplex hortensis), Paracress (Acmella oleracea), Pea sprouts/leaves (Pisum sativum), Polk (Phytolacca americana), Radicchio (Cichorium intybus), Samphire (Crithmum maritimum), Sea beet (Beta vulgaris subsp. maritima), Seakale (Crambe maritima), Siena Leone bologi (Crassocephalum spp.), Soko (Celosia argentea), Sorrel (Rumex acetosa), Spinach (Spinacia oleracea), Summer purslane (Portulaca oleracea), Swiss chard (Beta vulgaris subsp. cicla var. flavescens), Tatsoi (Brassica rapa Rosularis group), Turnip greens (Brassica rapa Rapifera group), Watercress (Nasturtium officinale), Water spinach (Ipomoea aquatica), Winter purslane (Claytonia perfoliata), Yarrow (Achillea millefolium)]; fruiting and flowering vegetables, such as those from trees [e.g., Avocado (Persea americana), Breadfruit (Artocarpus altilis)]; or from annual or perennial plants [e.g., Acorn squash (Cucurbita pepo), Armenian cucumber (Cucumis melo Flexuosus group), Aubergine (Solanum melongena), Bell pepper (Capsicum annuum), Bitter melon (Momordica charantia), Caigua (Cyclanthera pedata), Cape Gooseberry (Physalis peruviana), Capsicum (Capsicum annuum), Cayenne pepper (Capsicum frutescens), Chayote (Sechium edule), Chili pepper (Capsicum annuum Longum group), Courgette (Cucurbita pepo), Cucumber (Cucumis sativus), Eggplant (Solanum melongena), Luffa (Luffa acutangula, Luffa aegyptiaca), Malabar gourd (Cucurbita ficifolia), Parwal (Trichosanthes dioica), Pattypan squash (Cucurbita pepo), Perennial cucumber (Coccinia grandis), Pumpkin (Cucurbita maxima, Cucurbita pepo), Snake gourd (Trichosanthes cucumerina), Squash aka marrow (Cucurbita pepo), Sweet corn aka corn; aka maize (Zea mays), Sweet pepper (Capsicum annuum Grossum group), Tinda (Praecitrullus fistulosus), Tomatillo (Physalis philadelphica), Tomato (Lycopersicon esculentum var), Winter melon (Benincasa hispida), West Indian gherkin (Cucumis anguria), Zucchini (Cucurbita pepo)]; the flower buds of perennial or annual plants [e.g., Artichoke (Cynara cardunculus, C. scolymus), Broccoli (Brassica oleracea), Cauliflower (Brassica oleracea), Squash blossoms (Cucurbita spp.); podded vegetables [e.g., American groundnut (Apios americana), Azuki bean (Vigna angularis), Black-eyed pea (Vigna unguiculata subsp. unguiculata), Chickpea (Cicer arietinum), Common bean (Phaseolus vulgaris), Drumstick (Moringa oleifera), Dolichos bean (Lablab purpureus), Fava bean (Vicia faba), Green bean (Phaseolus vulgaris), Guar (Cyamopsis tetragonoloba), Horse gram (Macrotyloma uniflorum), Indian pea (Lathyrus sativus), Lentil (Lens culinaris), Lima Bean (Phaseolus lunatus), Moth bean (Vigna acontifolia), Mung bean (Vigna radiata), Okra (Abelmoschus esculentus), Pea (Pisum sativum), Peanut (Arachis hypogaea), Pigeon pea (Cajanus cajan), Ricebean (Vigna umbellata), Runner bean (Phaseolus coccineus), Soybean (Glycine max), Tarwi (tarhui, chocho; Lupinus mutabilis), Tepary bean (Phaseolus acutifolius), Urad bean (Vigna mungo), Velvet bean (Mucuna pruriens), Winged bean (Psophocarpus tetragonolobus), Yardlong bean (Vigna unguiculata subsp. sesquipedalis)]; bulb and stem vegetables [e.g., Asparagus (Asparagus officinalis), Cardoon (Cynara cardunculus), Celeriac (Apium graveolens var. rapaceum), Celery (Apium graveolens), Elephant Garlic (Allium ampeloprasum var. ampeloprasum), Florence fennel (Foeniculum vulgare var. dulce), Garlic (Allium sativum), Kohlrabi (Brassica oleracea Gongylodes group), Kurrat (Allium ampeloprasum var. kurrat), Leek (Allium porrum), Lotus root (Nelumbo nucifera), Nopal (Opuntia ficus-indica), Onion (Allium cepa), Prussian asparagus (Ornithogalum pyrenaicum), Shallot (Allium cepa Aggregatum group), Welsh onion (Allium fistulosum), Wild leek (Allium tricoccum)]; root and tuberous vegetables [e.g., Ahipa (Pachyrhizus ahipa), Arracacha (Arracacia xanthorrhiza), Bamboo shoot (Bambusa vulgaris and Phyllostachys edulis), Beetroot (Beta vulgaris subsp. vulgaris), Black cumin (Bunium persicum), Burdock (Arctium lappa), Broadleaf arrowhead (Sagittaria latifolia), Camas (Camassia), Canna (Canna spp.), Carrot (Daucus carota), Cassaya (Manihot esculenta), Chinese artichoke (Stachys affinis), Daikon (Raphanus sativus Longipinnatus group), Earthnut pea (Lathyrus tuberosus), Elephant Foot yam (Amorphophallus paeoniifolius), Ensete (Ensete ventricosum), Ginger (Zingiber officinale), Gobo (Arctium lappa), Hamburg parsley (Petroselinum crispum var. tuberosum), Jerusalem artichoke (Helianthus tuberosus), Jicama (Pachyrhizus erosus), Parsnip (Pastinaca sativa), Pignut (Conopodium majus), Plectranthus (Plectranthus spp.), Potato (Solanum tuberosum), Prairie turnip (Psoralea esculenta), Radish (Raphanus sativus), Rutabaga (Brassica napus Napobrassica group), Salsify (Tragopogon porrifolius), Scorzonera (Scorzonera hispanica), Skirret (Sium sisarum), Sweet Potato or Kumara (Ipomoea batatas), Taro (Colocasia esculenta), Ti (Cordyline fruticosa), Tigernut (Cyperus esculentus), Turnip (Brassica rapa Rapifera group), Ulluco (Ullucus tuberosus), Wasabi (Wasabia japonica), Water chestnut (Eleocharis dulcis), Yacon (Smallanthus sonchifolius), Yam (Dioscorea spp.)]; and even sea vegetables [e.g., Aonori (Monostroma spp., Enteromorpha spp.), Carola (Callophyllis variegata), Dabberlocks aka badderlocks (Alaria esculenta), Dulse (Palmaria palmata), Gim (Porphyra spp.), Hijiki (Hizikia fusiformis), Kombu (Laminaria japonica), layer (Porphyra spp.), Mozuku (Cladosiphon okamuranus), Nori (Porphyra spp.), Ogonori (Gracilaria spp.), Sea grape (Caulerpa spp.), Seakale (Crambe maritima), Sea lettuce (Ulva lactuca), Wakame (Undaria pinnatifida)], some of which are not even plants in the taxonomic sense.

Of particular interest for the method described herein are compounds and compositions derived from berry fruits, which are recognized as producing a wide array of (beneficial) phytochemicals. The botanical definition of a berry is a simple fruit produced from a single ovary, such as a grape. The berry is the most common type of fleshy fruit in which the entire ovary wall ripens into an edible pericarp. The flowers of these plants have a superior ovary formed by the fusion of two or more carpels. The seeds are embedded in the flesh of the ovary. However, the term “berry” as used herein is broader than the botanical definition and encompasses, for instance, false berries (e.g., blueberries), aggregate fruits (e.g., blackberries and raspberries), drupes (e.g., hackberries and Açaí palm), and accessory fruits (e.g., strawberries).

Examples of true berries include: grape (Vitis vinifera), tomato (Lycopersicon esculentum and other species of the family Solanaceae, many of which are commercial importance, such as Capsicum, and aubergine/eggplant (Solanum melongena), wolfberry or Goji berries (Lycium barbarum, Lycium spp.; Solanaceae), garberry (Berberis; Berberidaceae), red, black, and white currant (Ribes spp.; Grossulariaceae), elderberry (Sambucus niger; Caprifoliaceae), gooseberry (Ribes spp.; Grossulariaceae), honeysuckle (Lonicera spp.; Caprifoliaceae) (the berries of some species (e.g., honeyberries) are edible, and even though others are poisonous they may provide useful phytochemicals if properly purified), mayapple (Podophyllum spp.; Berberidaceae), nannyberry or sheepberry (Viburnum spp.; Caprifoliaceae), Oregon-grape (Mahonia aquifolium; Berberidaceae), and sea-buckthorn (Hippophae rhamnoides; Elaeagnaceae). Also contemplated herein within the term “berries” are the modified, juicy berries, such as the fruit of citrus. Such fruits, including orange, kumquat, grapefruit, lime, and lemon, are modified berries referred to botanically as hesperidium.

Also specifically contemplated herein is the chokeberry (Aronia melanocarpa; commonly called black chokeberry), which has attracted scientific interest due to its deep purple, almost black pigmentation that arises from dense contents of phenolic phytochemicals, and especially anthocyanins. Total anthocyanin content in chokeberries is 1480 mg per 100 g of fresh berries, and proanthocyanidin concentration is 664 mg per 100 g (Wu et al., J Agric Food Chem. 52: 7846-7856, 2004; Wu et al., J Agric Food Chem. 54: 4069-4075, 2006). Both values are among the highest measured in plants to date. Chokecherry produces these pigments mainly in the skin of the berries to protect the pulp and seeds from constant exposure to ultraviolet radiation (Simon, HortScience 32(1):12-13, 1997). By absorbing UV rays in the blue-purple spectrum, pigments filter intense sunlight. Scientific measurement of ORAC antioxidant strength demonstrates chokeberry with one of the highest values yet recorded—16,062 micromoles of Trolox equivalents per 100 g (Nutrient Data Laboratory, Agriculture Research Service, US Department of Agriculture, 2007 publication entitled “Oxygen Radical Absorbance Capacity (ORAC) of Selected Foods,” available on-line; see this ORAC reference also provides antioxidant scores for 277 common foods). Analysis of anthocyanins in chokeberries has identified the following individual chemicals (among hundreds known to exist in the plant kingdom): cyanidin-3-galactoside, epicatechin, caffeic acid, quercetin, delphinidin, petunidin, pelargonidin, peonidin, and malvidin. All these are members of the flavonoid category of antioxidant phenolics, and they are found in myriad other plants in differing concentrations.

Many “berries” as referenced herein are not true berries by the scientific definition, but are in fact drupes, epigynous fruits, or compound fruits. Drupes are fruits produced from a single-seeded ovary or achene; example drupes are hackberry (Celtis spp.; Cannabaceae) and Açaí (Euterpe), a palm fruit native to the Amazon region. Epigynous fruits are berry-like fruits formed from inferior ovaries, in which the receptacle is included. Notable examples are the fruits of the Ericaceae, including blueberry, huckleberry, and cranberry. Other epigynous fruits include: bearberry (Arctostaphylos spp.), crowberry (Empetrum spp.), lingonberry (Vaccinum vitis-idaea), strawberry tree (Arbutus unedo), and sea grape (Coccoloba uvifera; Polygonaceae). The fruit of cucumbers, melons and their relatives are modified berries called “pepoes.” Compound fruits are groups or aggregates of multiple individual fruits with seeds from different ovaries of a single flower, and include: blackberry, dewberry, boysenberry, olallieberry, and tayberry (genus Rubus), cloudberry (Rubus chamaemorus), loganberry (Rubus loganobaccus), raspberry, Rubus idaeus and other species of Rubus, salmonberry (Rubus spectabilis), thimbleberry (Rubus parviflorus), wineberry (Rubus phoenicolasius), bayberry, and boysenberry. Multiple fruit are the fruits of separate flowers, packed closely together, such as the mulberry. Others are accessory fruit, where the edible portion is not generated by the ovary, such as the strawberry.

Berry colors are due to natural plant pigments. Many are polyphenols such as the flavonoids, anthocyanins, and tannins localized mainly in berry skins and seeds. Berry pigments are usually antioxidants and thus have oxygen radical absorbance capacity (“ORAC”) that is high among plant foods (Wu et al., J. Agric. Food Chem. 52(12):4026-4037, 2004). Together with good nutrient content, ORAC distinguishes several berries within a new category of functional foods called “superfruits” and is identified by DataMonitor as one of the top 10 food categories for growth in 2008 (Food Navigator-USA.com, “Fresh, super and organic top trends for 2008”, Nov. 28, 2007).

Additional sources for modulating compounds, and methods for preparing compositions containing such, can be found in the literature. See, for instance, European published application EP 1,985,280; Schmid et al., “Plant Stem Cell Extract for Longevity of Skin and Hair” SOFW-Journal, 134:30-35, 2008; U.S. Pat. Nos. 7,544,497, 7,582,674; and International Patent Publication No. WO/2007/084861.

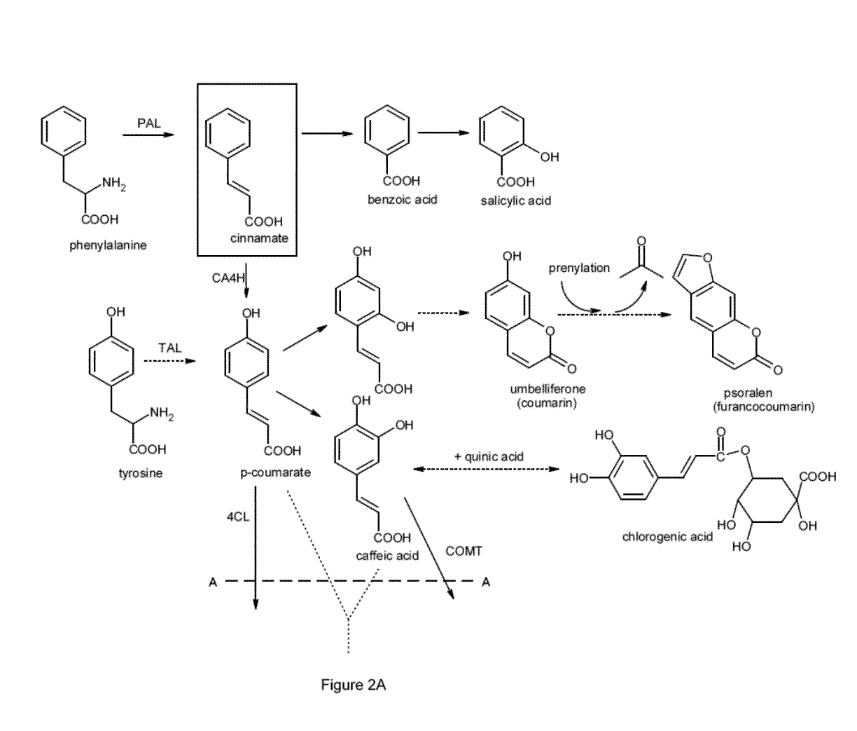

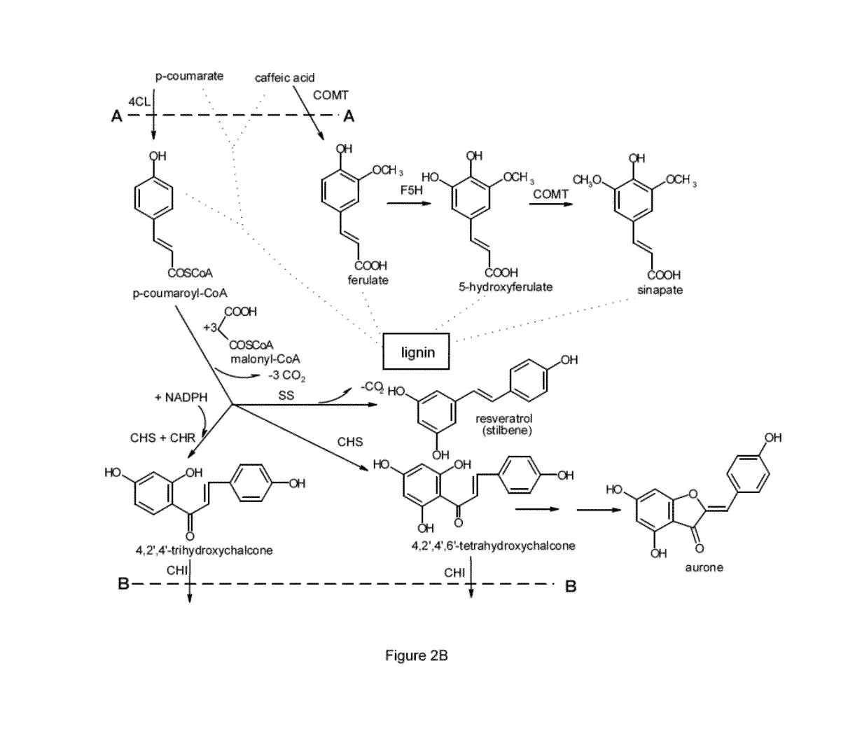

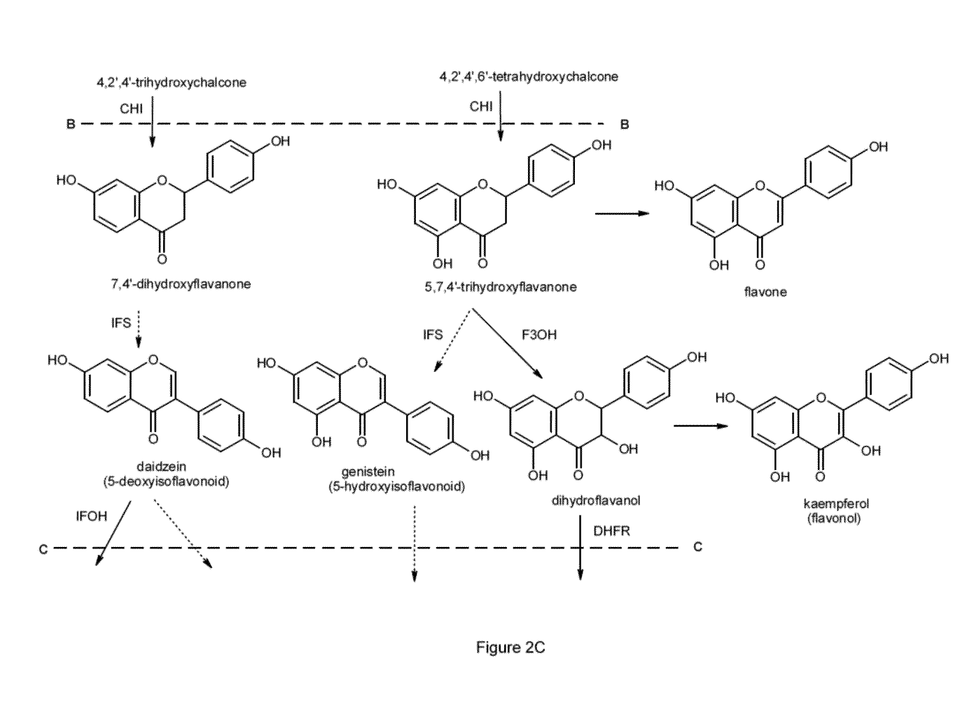

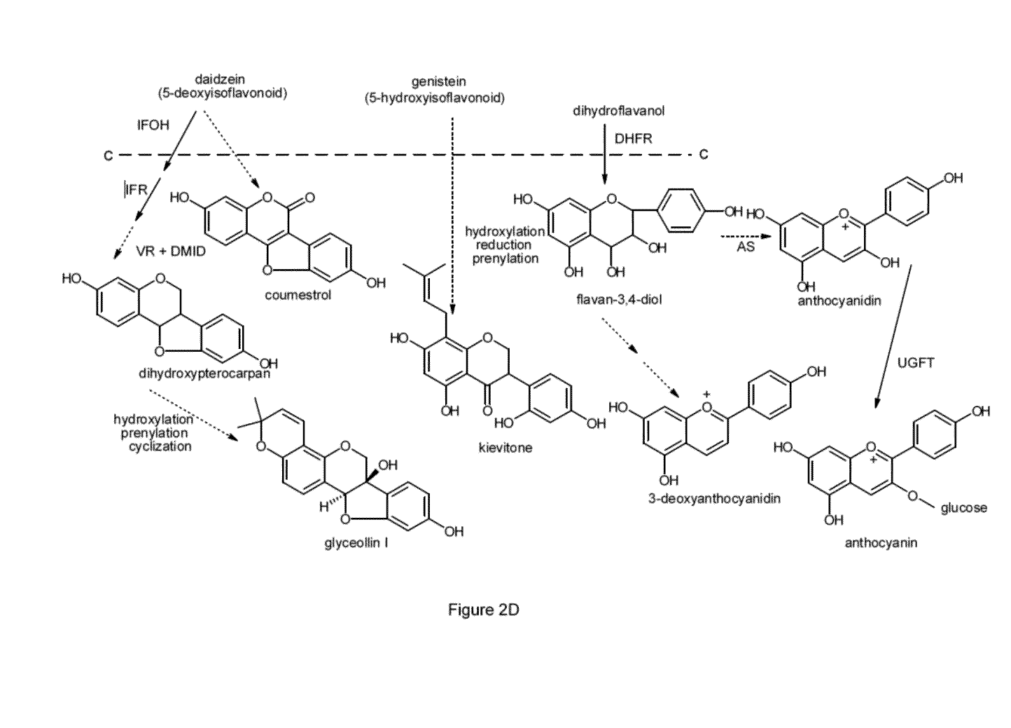

Further exemplary modulating compounds include for instance stress-induced phenylpropanoids (see, e.g., FIG. 2 and Dixon et al., The Plant Cell 7:1085-1097, 1995).

Exemplary modulating compounds or agents include those selected from the group of compounds contained in coffee cherry acids or extracts including the antioxidant compounds chlorogenic acid, quinic acid, caffeic acid, ferulic acid and proanthocyanidins.

Exemplary modulating compounds or agents include ubiquinone, idebenone and the analogs and derivatives thereof including various esters and conjugated compounds.

Exemplary modulating compounds or agents include extracts and the analogs and derivatives obtained from cocoa. The extracts, compounds or combinations of compounds derived from the cocoa beans from various isolation or purification processes are derived from any species of Theobroma, Herrania or inter- or intra-species hybrid crosses thereof. It is also understood that similarly such extracts or compounds are included if derived from genetically engineered versions of these species or hybrids. Furthermore synthetic formulations, analogs or derivatives of these compounds are similarly included as well as compounds derived from natural or synthetic fermentation processes. These extracts or compounds preferably comprise polyphenol(s) such as cocoa procyanidin(s), such as at least one cocoa procyanidin selected from (+) catechin, (−) epicatechin, procyanidin oligomers 2 through 18, procyanidin B-5, procyanidin B-2, procyanidin A-2 and procyanidin C-1.

Exemplary modulating compounds or agents include extracts and the analogs and derivatives obtained from Camellia sinensis, Camellia sinensis sinensis, Camellia sinensis assamica or Camellia oleifera either naturally or synthetically derived.

Exemplary modulating compounds or agents include resveratrol and the analogs and derivatives thereof, including viniferin, gnetin H, and suffruticosol B.

For methods of preparing (green) tea extracts, see, for instance, Perva-Uzunalic et al., Food Chemistry 96(4):597-605, 2006; Koiway & Masuzawa, Jpn. J. Appl. Phys 46:4936-4938, 2007; U.S. Pat. Nos. 4,668,525 and 3,080,237. Tea extracts containing polyphenols, as well as individual tea-derived polyphenols, are commercially available from many sources. By way of example only, one source is Pharma Cosmetix Research, LLC (Richmond, Va.), the supplier of Premier Green Tea Extract Lot#10783 that was used in various examples described herein.

Idebenone (CAS no. 58186-27-9) is commercially available from myriad suppliers, including for instance Pharma Cosmetix Research, LLC (Richmond, Va.), the supplier of idebenone Lot #27816 that was used in various examples described herein.

Coffee cherry extract can be prepared using art recognized methods; see, for instance U.S. Patent Publication No. 2007/0281048 (published Dec. 6, 2007). In addition, the coffee cherry extract referred to as COFFEEBERRY® can be purchased from VDF FutureCeuticals, Inc. (Momence, Ill.); for several of the experiments described herein, COFFEEBERRY® Beauty Lot#02480000×5729 from VDF was used.

In one embodiment a modulating compound or combination of modulating compounds may be used to extend the lifespan of one or more types of cells in the skin or subcutaneous tissue under the skin including fat, fascia, muscle and blood vessels. Such a treatment may be topical or systemic and may be delivered, with or without penetration enhancing agents or therapies, in many forms well known to one skilled in the art of skin medications.

Topical delivery of modulating agents may uniquely extend the lifespan of contacted cells in the skin or subcutaneous tissues without necessarily modulating the lifespan of the entire organism. Systemic delivery of modulating agents may also reach the skin to produce a lifespan modulating effect.

Topical formulations may include but are not limited to creams, emollients, gels, lotions, solutions, micro-emulsions, suspensions, ointments, spray mists, delayed or time release formulations, patches, injectable, implantable, depot, mask or other formulations.

Various methods may be utilized to enhance penetration including liposomal or polymer or other matrix delivery systems, agents which enhance delivery or disrupt skin barrier function, ultrasound or acoustic assisted delivery, laser or mechanical disruption of the skin or other energy based devices which enhance delivery or disrupt skin barrier function thus indirectly enhancing delivery into the skin or through the skin into the subcutaneous tissues.

Other embodiments may include delivery combined with skin care products such as cosmetic foundations, makeup, lipstick, shampoo, cleansers, sunscreen, and body lotions. Systemic delivery may include but not limited to oral, parenteral, intravenous, intradermal, intramuscular, rectal, buccal, sublingual, vaginal, ophthalmic, otic, intranasal, nebulizer, injectable, depot, catheter, endoscopic or incorporated onto or into implantable devices or agents.

In one embodiment the modulating agent is used to reduce one or more factors which create the appearance of aging or prematurely aging skin such as fine lines, wrinkles, uneven pigment, skin radiance, skin elasticity, skin thickness, pore size, skin sagging, loss of subcutaneous fat or volume in the skin collagen and abnormal vascularity.

In yet another embodiment the modulating agent may be used in combination with agents or methods which protect the skin from UV ultraviolet or IR infrared damage from any light source to enhance, facilitate or produce DNA repair or telomere structure protection or repair or to prevent, diminish or avoid apoptosis. The concomitant use either simultaneously in the same formulation or serially within 24 hours of compounds which function as antioxidants may be utilized.

A preferred embodiment may include the use of an agent which modulates the maintenance of telomere in combination with an agent which mimics caloric restriction, such as resveratrol or a sirtuin pathway modulating agent.

Other topical embodiments may utilize modulation of lifespan to decrease or shorten the lifespan of cancerous cells either alone or in combination with various anticancer agents or therapies to improve or enhance or increase the destruction of the cancer and thus improve the cure rate of such a therapy.

Yet another topical embodiment may target other structures in the skin such as hair or nails. One such application is to prevent, delay or reverse hair loss or other disorders of aging such as graying of the hair. The modulating agent may be used to contact the hair directly or it may modulate the aging or damage to the skin in which the hair follicle is located thus indirectly reducing hair loss.

One embodiment utilizes modulation to protect or repair the telomere structure so as to extend the lifespan of at least one cell. Alternatively modulation may be directed to shorten the lifespan of at least one cell. While extending the lifespan of living cells is one very important function of the invention, in certain cases such as diseased, damaged or cancerous cells it may be desirable to accelerate the death of such cells or to turn immortalized cells back into mortal cells so that they may be killed or be more responsive to other therapies.

There are various skin cells which may be targeted alone or in various combinations to contact a modulating compound. These include but are not limited to keratinocytes, fibroblasts, melanocytes, Langerhans cells, merkel cells, nerve cells, endothelial cells, adipocyte or fat cells, muscle cells and the various specialized cells of sweat and oil glands, hair structure cells, nail and other skin appendage cells. It may also be desirable to modulate the lifespan of stem and progenitor cells. Subcellular organelles including mitochondria, ribosomes, and Golgi apparatus may also be indirect targets for the modulating agent as well as nuclear and mitochondrial DNA.

Another embodiment involves contacting at least one cell of the organism with a sufficient amount of modulating compound to protect, defend, reverse, rescue, revive, resuscitate, or repair acute stress from the environment, from oxidative stress, from acute or chronic injury or disease including acutely injured and dying cells. These cells may be skin cells or they may be cells from any or all parts of the tissue, organ or organism.

In yet another embodiment the modulating compound may be used to contact one or more cells when they are not present in the living organism but rather they are ex vivo such as an organ being prepared for transplant, or in vitro. For example a donor organ being transported is subjected to various cellular stresses and has a finite time span of viability and the modulating agent may be utilized to extend this time span and/or to increase the number of healthy functioning cells present during the same time span. Another application with transplanted cells, tissues or organs is to repair the telomere structure so that the lifespan is extended before or after transplantation. For example a donor kidney from an older donor might be treated with a modulating agent prior to transplantation in order to extend the lifespan of the kidney for transplant into a younger transplant recipient or to make the transplanted kidney less vulnerable to apoptosis or damage from either the procedure itself or to the immunosuppressive therapies given after the transplantation.

In vitro fertilization and embryo and stem cell research are yet other embodiments wherein the modulating agent may be used to extend the lifespan of cells.

In another embodiment the modulating agent may be used to extend the lifespan of the progeny or a cloned derivative of an organism. It is known that somatic and embryonic cloning may produce cloned organisms with shorter lifespan than the original organism that was cloned. The modulating agent may be used to extend the lifespan of a cloned organism directly. Another option is to use the modulating agent to repair the telomere structure in a recloning event either of the original organism or of the clone itself.

In one embodiment the modulating agent may contact plant cells which are being cloned or expanded by a meristematic process in which the cell line has become senescent and the agent may help restore viability to extend the lifespan of the plant cell culture allowing continued commercial production of copies of the plant.

A useful embodiment may utilize the modulating agent to treat autoimmune disease where autoimmune or inflammatory processes shorten the lifespan of cells thus producing disease, disability, premature aging or even death.

One preferred embodiment incorporates other lifespan modulating agents with the modulating agent or agents described in this invention for the purpose of extending the lifespan or shortening the lifespan of at least one cell. For example a modulating agent to shorten lifespan might be included with an anti cancer therapeutic agent or treatment with the purpose of making the cancerous target cell more vulnerable to the therapy. Another example would be to differentially modulate lifespan so that the lifespan of the cancerous cells was shortened but the lifespan of the non cancerous normal cells was extended or at least protected.

An embodiment to extend the lifespan of cardiac muscle cells could be used to extend the lifespan of the entire organism such as a human or an animal such as a horse or companion animal such as a cat or dog. The use of a modulating agent to prevent, diminish or reverse apoptosis in cardiac muscle cells during an acute injury such as a myocardial infarction or heart attack or ischemic episode not only preserves these cells but also may prevent disability or death of the entire organism.

Other embodiments include diverse and novel methods of producing contact of the modulating agent with a cell including aerosolizing into a steam sauna or humidifier for inhalation of the agent, impregnating clothing for contact with the agent, impregnating implantable devices such as vascular stents or joint replacements or ocular lens implants. Intraocular injections of a modulating agent might be used to extend the lifespan of retinal cells. Injectable filling agents are commonly used for the skin and subcutaneous tissue and a modulating agent may be incorporated into the implant or agent in a time or delayed release formulation. Transdermal patches for hormonal therapy might incorporate a modulating agent for systemic delivery as part of an anti aging hormone replacement therapy. Novel oral delivery may include incorporation into toothpaste, mouthwash, oral lozenges, chewable items, or dental floss.

An embodiment for oral delivery may include diverse forms known in the art including but not limited to nutritional supplements or vitamins, additives for food or beverages, in combination with various drugs. The incorporation of a modulating agent via genetically engineered plant or animal or other food products is another route to administer a modulating agent.

One key embodiment is the use of a modulating agent or compound in association with (either combined, co-administered, or sequentially administered) other lifespan modulating compounds. A telomere structure maintenance modulating compound may be combined in such a manner with an agent or compound which mimics or produces directly or indirectly caloric restriction biochemical and/or cellular processes in living organisms.

Another similar embodiment is the use of a modulating agent or compound in association with (either combined, co-administered, or sequentially administered) other lifespan modulating compounds which modulate the biogenesis and/or respiratory capacity of mitochondria.

Premature or accelerated aging as a result of direct or indirect interaction of cells with environmental factors which injure at least one cell or which produce cellular stress and/or cellular inflammatory processes and/or oxidative stress and/or DNA or telomere structure damage and/or cellular apoptosis may be delayed, retarded, diminished, prevented, or even repaired or reversed by use of effective combinations and concentrations of at least one of a telomere structure modulating compound, a caloric restriction mimicking compound and a compound which stimulates more efficient mitochondrial respiratory activity and/or an increase in the number of mitochondria.

In a further embodiment a telomere structure modulating compound may be utilized in combination with at least one mitochondrial biogenesis modulating compound so that not only is the telomere structure of either the mitochondrial DNA and/or the nuclear DNA protected but also the number of mitochondrial organelles is also modulated. To further the goal of lifespan extension the maintenance of the telomere structure would be stimulated, activated or enhanced as well as stimulating an increase in the actual number of mitochondria.

In a further embodiment, in the case of diseased or cancerous cells the opposite goal would be desirable in that accelerating the death of these abnormal cells would be the goal and thus impairing the telomere structure maintenance and decreasing the number or the respiratory efficiency of the diseased or cancerous cells would be desirable.

In a further embodiment it may be desirable to extend the lifespan of healthy cells and shorten the lifespan of diseased or cancerous cells in order to maximize the healthy lifespan of the tissue, organ or entire organism.

In one embodiment a modulating agent such as idebenone, or its derivatives or analogs which transfer electrons rather than terminate electron transfer, may be used to reduce oxidative stress on mitochondria by transferring electrons down the electron transport system within the mitochondria bypassing complex I and instead transferring the electron to complex III. Complex I creates much of the ROS and oxidative stress within the mitochondria that is internally generated (in contrast to ROS created by exposure to outside environmental stress or other injuries) and mitochondria have limited ability to neutralize oxidative stress thus mitochondria respiratory efficiency declines over time and is responsible for part of the premature aging or senescence or dysfunction or disease states in cells so affected. This bypass of electrons then may contribute to lifespan extension of the cell as well as contribute to a healthier lifespan.

In a further embodiment a modulating agent may improve or protect the function or even produce repair of damage to the ribosomes. Ribosomes are responsible for the translation of instructions from the DNA during the synthesis of proteins. Thus, maintaining the accuracy of ribosome translational activity may prolong lifespan. It has recently been reported that UVB radiation induces persistent activation of ribosome and oxidative phosphorylation pathways (Tsai et al., Radiat. Res. 171(6):716-724, 2009. Those authors noted that ultraviolet B (UVB) radiation has strong biological effects and modulates the expression of many genes. Though the major biological pathways affected by UVB radiation remain controversial, Tsai et al. used a loop-design microarray approach and applied rigorous statistical analyses to identify differentially regulated genes at 4, 8, 16 or 24 hours after UVB irradiation. The most prominent biological categories in lists of differentially regulated gene sets were extracted by functional enrichment analysis. With this approach, the authors determined that genes participating in two prime cellular processes, the ribosome pathway and the oxidative phosphorylation pathway, were persistently activated after UVB irradiation. Mitochondrial activity assays confirmed increased activity for up to 24 h after UVB irradiation. These results suggest that the persistent activation of ribosome and oxidative phosphorylation pathways may have a key role in UVB-radiation-induced cellular responses.

Also contemplated are methods that improve immune function, for instance by modifying the expression of one or more genes involved in a nitric oxide pathway. Synthesis of nitric oxide (NO) is one of the important effector functions of innate immune cells. Although several reports have indicated mistletoe lectins induce immune cells to produce cytokines, studies regarding the activities of the lectins in the production of NO have been very limited. It has recently been reported (Bong-Kang et al., J. Biomed. Sci., 197-204, 2007), for instance, that Korean mistletoe (e.g., Viscum album coloratum) lectin (KML-IIU) induces NO synthesis in a murine macrophage cell line. When the macrophage cells were treated with KML-IIU in the presence of a suboptimal concentration of IFN-gamma, NO production was induced in a concentration-dependent manner (Id.).

In a further embodiment modulating the rate of protein synthesis through ribosomal activity modulation may be utilized to increase the lifespan of a cell.

In a further embodiment a nucleic acid may be introduced into a cell to modulate the level of a modulating agent that is at least about 70%, 80%, 90%, 99% identical to the sequence of a modulating agent target such as telomerase or sirtuin or electron transport protein.

In another embodiment various methods of diagnosing the level of a telomere structure maintenance protein are utilized such methods which are well known to those skilled in the art of these diagnostics. Using such diagnosis one may determine if an organism is likely to have accelerated aging or shortened lifespan. After such a diagnosis is made, then a therapeutically effective amount of a modulating agent may be used to treat that organism. The efficacy of this treatment may then be measured again at periodic appropriate intervals to assess the progress of the treatment. Such diagnostic methods may also be used in screening compounds and formulations of compounds and efficacy of delivery methods and optimal concentrations of modulating agents.

In another embodiment such diagnostic information may be combined with various other data obtained from the organism in order to create a profile or index that gives a relative value scale for aging or lifespan for benchmarking an individual organism relative to a larger population of the same organism or to a historical database of the same organisms or any other subset of data which might be of interest. This index might be viewed as an aging index or an aging ageing index or a longevity or lifespan index.

In another embodiment this data from the index could be used to assess both the need for treatment intervention with a modulating agent, but also to guide the therapeutic treatment doses and routes of administration and protocols. It could also be used for risk assessment or for predictive applications. A lifespan extension factor or age protection or protective factor or an anti aging protection or protective factor could also be created to guide therapy or to assign a value to the efficacy of a modulating agent.

In an illustrative embodiment a human or animal is tested diagnostically for the level of a lifespan modulating protein or factor and then rated or graded relative to other human or like animal populations and how they compare relative to this group provides a relative risk factor for greater or shorter lifespan than the comparison group. A lifespan modulating compound or group of compounds might then be selected to treat the human or animal based on the lifespan extension factor. This could be used in an attempt to repair or correct existing damage or it could be used as a lifespan protective factor in a preventive way. Diagnostic testing could then be utilized to assess the efficacy of the treatment and guide ongoing therapeutic efforts using the modulating factor(s).

In another embodiment a buccal swab, punch or shave biopsy from the skin or any internal organ or system, for the purpose of assessment of anti aging gene expression profiles through the use of human genome, or specialized custom cDNA microarrays is collected and compared to a control sample, which can be from an age matched subject, a pooled collection of subjects or the same subject taken years earlier or later. The comparison of this profile would enable a determination to be made on the relative effects of aging on longevity/mitochondrial related genetic factors.

In a further embodiment a buccal swab, punch or shave biopsy from the skin or any internal organ or system, for the purpose of assessment of anti-aging gene expression profiles through the use of human genome, or specialized cDNA microarray, is collected from a treated and untreated location on the same subject. Comparison of this genome expression profile can be used to assess the ability of the treatment modality to alter/extend the longevity or mitochondrial function. This embodiment will allow for the testing of formulation levels, combinations of, and sequential application of modulating compounds as viable interventions. One example of this would be the inclusion of a modulating agent in a sunscreen that is applied to a subject and then tested and through the aforementioned genomic data a relative level of efficacy can be determined.

### Compositions, Including Pharmaceutical Compositions

Compositions for use in accordance with the present methods may be formulated in conventional manner using one or more physiologically acceptable carriers. Methods and formats for cosmetic and cosmeceutical compositions are well known. For non-limiting examples, see for instance US publication no. 2009/0208433, Japan publications no. JP08092057, JP2000319154; and United Kingdom publication no. GB2445265A.

Compounds and their physiologically acceptable salts and solvates may be formulated for administration by, for example, injection, inhalation or insufflation (either through the mouth or the nose) or oral, buccal, parenteral or rectal administration. The compound is administered locally, at the site where the target cells, e.g., diseased or aged cells, are present.

Compounds can be formulated for a variety of dispensation methods, including systemic (injectable, pill form, suppository, inhalant) and topical (creams, lotions, gel, wrap, coated bandage or adhesive strip) or localized administration. For systemic administration, injection is preferred, including intramuscular, intravenous, intraperitoneal, and subcutaneous. The injectable can be formulated in liquid solutions, preferably in physiologically compatible buffers such as Ringer's solution. In addition, the compounds may be formulated in solid form and redissolved or suspended immediately prior to use. Lyophilized forms are also included.

For oral administration, compositions may take the form of, for example, tablets, lozenges, or capsules prepared by conventional means with pharmaceutically acceptable excipients. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds may be conveniently delivered in the form of an aerosol spray presentation from pressurized packs or a nebulizer. In the case of a pressurized aerosol the dosage unit may be determined by providing a valve to deliver a metered amount. The compound can be prepped for use in an inhaler or insufflator and may be formulated containing a powder mix of the compound.

The compounds may be formulated for parenteral administration by injection, e.g., by bolus injection or continuous infusion. The compositions may contain formulatory agents such as suspending, stabilizing and/or dispersing agents. Alternatively, the active ingredient may be in powder form for constitution with a suitable vehicle, e.g., sterile pyrogen-free water, before use.

Slow release implantable formulations may include coated devices such as vascular stents or grafts, dermal or subcutaneous implants, cervical rings, dental implants or other implant or infusion pump delivery methods.

The compounds may also be formulated in rectal compositions such as suppositories or retention enemas.

In addition to the formulations described previously, the compounds may also be formulated as a depot preparation. Such long acting formulations may be administered by implantation (for example subcutaneously or intramuscularly) or by intramuscular injection.

The compound(s) may also be formulated so that subcutaneous delivery through application or addition of ultrasound, iontophoresis, occlusion, sonication and/or other mechanisms that enlarge the pore size, disrupt the epidermal barrier, alter the chemical structure or otherwise drive the compound(s) further through, or enhance the absorption of, the skin than could be expected from application of the formulation alone. These processes may also enhance the effect of the compound(s) through increased absorption or chemical/physical change making the compound(s) more reactive or effective.

Pharmaceutical compositions (including cosmetic preparations) may comprise from about 0.00001 to 100%, such as from 0.001 to 10% or from 0.1% to 5% by weight or volume of one or more compounds described herein, such as for instance coffee cherry, idebenone, carnosine, green tea extract, or another plant extract or component thereof.

In one embodiment, a compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin.

Formulations may be colorless, odorless ointments, lotions, creams, micro-emulsions and gels.

Compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight.

Compounds may be incorporated into lotions, which generally are preparations to be applied to the skin surface without friction, and are typically liquid or semi liquid preparations in which solid particles, including the active agent, are present in a water or alcohol base. Lotions are usually suspensions of solids, and may comprise a liquid oily emulsion of the oil-in-water type.

Compounds may be incorporated into creams, which generally are viscous liquid or semisolid emulsions, either oil-in-water or water-in-oil. Cream bases are water-washable, and contain an oil phase, an emulsifier and an aqueous phase.

Compounds may be incorporated into micro-emulsions, which generally are thermodynamically stable, isotropically clear dispersions of two immiscible liquids, such as oil and water, stabilized by an interfacial film of surfactant molecules.

Compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent together and mixing until a characteristic semisolid product is produced. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives.

Other active agents may also be included in formulations, e.g., other anti-inflammatory agents, analgesics, antimicrobial agents, antifungal agents, antibiotics, vitamins, antioxidants, and sun block agents commonly found in sunscreen formulations.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. Novel pumps or dispensers which mix products from separate chambers at the time of dispensing may be used. When the composition is a cream, it can simply be stored in a non-deformable bottle or squeeze container, such as a tube or a lidded jar. The composition may also be included in capsules such as those described in U.S. Pat. No. 5,063,507.

In an alternative embodiment, a pharmaceutical formulation is provided for oral or parenteral administration, in which case the formulation may comprises an activating compound-containing microemulsion as described above, but may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Conditions can be treated or prevented by, e.g., systemic, topical, intraocular injection of a compound described herein, or by insertion of a sustained release device that releases a compound described herein. Polymers can be used for controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of lipid-capsulated compounds (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

Compounds described herein may be stored in oxygen free environment according to methods in the art. For example, resveratrol or analog thereof can be prepared in an airtight capsule for oral administration.

Cells, e.g., treated ex vivo with a compound described herein, can be administered according to methods for administering a graft to a subject.

It is also contemplated that the compositions described herein can be used in combination with other compounds or drugs, for instance other recognized antioxidant compounds, sunscreens, anticancer and anti-infective agents, anti-inflammatory substances, and so forth.

### Arrays

Also provided herein are collections of genes that have been found to be influenced by antioxidant(s), and/or that are now recognized as being involved in lifespan extension, cell longevity or health, mitochondrial biogenesis or function, telomere maintenance or DNA fidelity or repair, and so forth. The identification of sets of genes that are responsive to antioxidant treatment and that act in a concerted manner (e.g., in a recognized pathway, in a similar manner as to magnitude and/or direction of change in gene expression, etc.) enables the production of tailored arrays. Such arrays can be used in myriad ways, including but not limited to characterizing the activities of known antioxidants, studying and identifying potential new antioxidant compositions, tracking the biological effect (e.g., on an experimental system or a subject) of an antioxidant treatment regimen, and analysis of, e.g., skin biopsy, blood, and other various body components.

The specific arrays described herein were constructed at the inventor's specifications by SABiosciences (Fredrick, Mass.) (information relevant to their procedures is available on-line at sabiosciences.com/customarray_biomarker.php#hiw). The genes in the first custom microarray (“Array 1”) were selected based on an exhaustive literature search for previously recognized longevity genes and lifespan altering genes. The second microarray (Array 2) includes the genes from the first array, plus genes related to mitochondrial biogenesis, respiration efficiency, telomere maintenance, and genes that had a large significant response the Agilent and/or Affymetrix Human Genome array analyses described herein. This customization of the array permits focused genetic analysis that is significantly faster than analyzing the entire human genome. The array style selected was a 96 well plate suited for a BioRad iCycler. The initial array was a 48 gene set (including all required controls and QC checks recommended by the manufacturer) and allowed two samples to be run on each plate. The second array had 91 genes of interest (the remaining spaces were controls and QC checks). The genes were selected using the SABioscience custom array online design tool, which gave a RefSeq number once the gene symbol of interest was entered.

By way of example, the following lists provide two different arrays of genes identified herein as being associated with or linked to some aspect of lifespan extension. Design and use of these exemplified arrays are described in the Examples.

Array 1 (Gene symbols)

| | | | | | | |
|---|---|---|---|---|---|---|
| ACE | CCL4L1 | GH1 | IFI44 | MAPK14 | PTGS2 | TNF |
| ACTB | CLK1 | HIGX1A | IGF1 | NADSYN1 | SHC1 | TP53 |
| APOE | COX1 | HLA-DRA | IGF2 | NFKB1 | SIRT1 | |
| BAX | CREBBP | HPRT1 | IL10 | NOS2A | SOD1 | |
| BCL2 | CYP19A1 | HSPA1A | IL1A | PARP1 | SOD2 | |
| CASP2 | DDC | HSPA1B | IL6 | PARP2 | TEP1 | |
| CASP9 | GAPDH | HSPA1L | KRAS | PPARG | TERT | |

Array 2

| Gene Symbol | Alias | Refseq # | Official Full Name |
|---|---|---|---|
| ACE | ACE1/CD143/DCP/DCP1/MGC26566/MVC | NM_000789 | Angiotensin I converting enzyme (peptidyl- |
| | D3 | | dipeptidase A) 1 |
| ACTB | PS1TP5BP1 | NM_001101 | Actin, beta |
| APOE | AD2/LPG/MGC1571 | NM_000041 | Apolipoprotein E |
| BAX | BCL2L4 | NM_004324 | BCL2-associated X protein |
| BCL2 | Bcl-2 | NM_000633 | B-cell CLL/lymphoma 2 |
| BCL2L1 | BCL-XL/S/BCL2L/BCLX/Bcl- | NM_138578 | BCL2-like 1 |
| | X/DKFZp781P2092/bcl-xL/bcl-xS | | |
| BMP2 | BMP2A | NM_001200 | Bone morphogenetic protein 2 |
| CASP2 | CASP-2/ICH-1L/ICH-1L/1S/ICH1/NEDD2 | NM_032982 | Caspase 2, apoptosis-related cysteine peptidase |
| CASP9 | APAF-3/APAF3/CASPASE-9c/ICE- | NM_001229 | Caspase 9, apoptosis-related cysteine peptidase |
| | LAP6/MCH6 | | |
| CCL4L1 | AT744.2/CCL4L/LAG-1/LAG1/SCYA4L | NM_001001435 | Chemokine (C-C motif) ligand 4-like 1 |
| CDKN2A | ARF/CDK4I/CDKN2/CMM2/INK4/INK4a/ | NM_000077 | Cyclin-dependent kinase inhibitor 2A |
| | MLM/MTS1/TP16/p14/p14ARF/p16/p16IN | | (melanoma, p16, inhibits CDK4) |
| | K4/p16INK4a/p19 | | |
| CLK1 | CLK/CLK/STY/STY | NM_004071 | CDC-like kinase 1 |
| COL1A1 | OI4 | NM_000088 | Collagen, type I, alpha 1 |
| COL3A1 | EDS4A/FLJ34534 | NM_000090 | Collagen, type III, alpha 1 |
| COX1 | MTCO1 | NP_536845 | Cytochrome c oxidase I |
| CREBBP | CBP/KAT3A/RSTS | NM_004380 | CREB binding protein |
| CRP | MGC149895/MGC88244/PTX1 | NM_000567 | C-reactive protein, pentraxin-related |
| CYP19A1 | ARO/ARO1/CPV1/CYAR/CYP19/MGC104 | NM_000103 | Cytochrome P450, family 19, subfamily A, |
| | 309/P-450AROM | | polypeptide 1 |
| DDC | AADC | NM_000790 | Dopa decarboxylase (aromatic L-amino acid |
| | | | decarboxylase) |
| DUSP2 | PAC-1/PAC1 | NM_004418 | Dual specificity phosphatase 2 |
| EGF | HOMG4/URG | NM_001963 | Epidermal growth factor (beta-urogastrone) |
| EGR2 | AT591/CMT1D/CMT4E/DKFZp686J1957/F | NM_000399 | Early growth response 2 |
| | LJ14547/KROX20 | | |
| FOS | AP-1/C-FOS | NM_005252 | V-fos FBJ murine osteosarcoma viral oncogene |
| | | | homolog |
| FOXO3 | AF6q21/DKFZp781A0677/FKHRL1/FKHR | NM_001455 | Forkhead box O3 |
| | L1P2/FOXO2/FOXO3A/MGC12739/MGC3 | | |
| | 1925 | | |
| GAPDH | G3PD/GAPD/MGC88685 | NM_002046 | Glyceraldehyde-3-phosphate dehydrogenase |
| GCH1 | DYT14/DYT5/GCH/GTP-CH-1/GTPCH1 | NM_000161 | GTP cyclohydrolase 1 |
| GH1 | GH/GH-N/GHN/hGH-N | NM_000515 | Growth hormone 1 |
| GPX1 | GSHPX1/MGC14399/MGC88245 | NM_000581 | Glutathione peroxidase 1 |
| HBEGF | DTR/DTS/DTSF/HEGFL | NM_001945 | Heparin-binding EGF-like growth factor |
| HGDC | HIGX1A | SA_00105 | Human Genomic DNA Contamination |
| HLA- | HLA-DRA1 | NM_019111 | Major histocompatibility complex, class II, DR |
| DRA | | | alpha |
| HMOX1 | HO-1/HSP32/bK286B10 | NM_002133 | Heme oxygenase (decycling) 1 |
| HPRT1 | HGPRT/HPRT | NM_000194 | Hypoxanthine phosphoribosyltransferase 1 |
| HSPA1A | FLJ54303/FLJ54370/FLJ54392/FLJ54408/F | NM_005345 | Heat shock 70 kDa protein 1A |
| | LJ75127/HSP70-1/HSP70- | | |
| | 1A/HSP70I/HSP72/HSPA1/HSPA1B | | |
| HSPA1B | FLJ54328/HSP70-1B/HSP70-2/HSPA1A | NM_005346 | Heat shock 70 kDa protein 1B |
| HSPA1L | HSP70-1L/HSP70-HOM/HSP70T/hum70t | NM_005527 | Heat shock 70 kDa protein 1-like |
| HSPA6 | — | NM_002155 | Heat shock 70 kDa protein 6 (HSP70B′) |
| IFI44 | MTAP44/p44 | NM_006417 | Interferon-induced protein 44 |
| IGF1 | IGF1A/IGFI | NM_000618 | Insulin-like growth factor 1 (somatomedin C) |
| IGF2 | C11orf43/FLJ22066/FLJ44734/INSIGF/pp99 | NM_000612 | Insulin-like growth factor 2 (somatomedin A) |
| | 74 | | |
| IL10 | CSIF/IL- | NM_000572 | Interleukin 10 |
| | 10/IL10A/MGC126450/MGC126451/TGIF | | |
| IL11 | AGIF/IL-11 | NM_000641 | Interleukin 11 |
| IL1A | IL-1A/IL1/IL1-ALPHA/IL1F1 | NM_000575 | Interleukin 1, alpha |
| IL33 | C9orf26/DKFZp586H0523/DVS27/NF- | NM_033439 | Interleukin 33 |
| | HEV/NFEHEV/RP11-575C20.2 | | |
| IL6 | BSF2/HGF/HSF/IFNB2/IL-6 | NM_000600 | Interleukin 6 (interferon, beta 2) |
| IL8 | CXCL8/GCP- | NM_000584 | Interleukin 8 |
| | 1/GCP1/LECT/LUCT/LYNAP/MDNCF/MO | | |
| | NAP/NAF/NAP-1/NAP1 | | |
| IMMP1L | FLJ25059/IMP1/IMP1-LIKE | NM_144981 | IMP1 inner mitochondrial membrane peptidase- |
| | | | like (S. cerevisiae) |
| JUN | AP-1/AP1/c-Jun | NM_002228 | Jun oncogene |
| KIT | C-Kit/CD117/PBT/SCFR | NM_000222 | V-kit Hardy-Zuckerman 4 feline sarcoma viral |
| | | | oncogene homolog |
| KL | — | NM_004795 | Klotho |
| KRAS | C-K-RAS/K-RAS2A/K-RAS2B/K- | NM_004985 | V-Ki-ras2 Kirsten rat sarcoma viral oncogene |
| | RAS4A/K-RAS4B/KI- | | homolog |
| | RAS/KRAS1/KRAS2/NS3/RASK2 | | |
| MAPK14 | CSBP1/CSBP2/CSPB1/EXIP/Mxi2/PRKM1 | NM_001315 | Mitogen-activated protein kinase 14 |
| | 4/PRKM15/RK/SAPK2A/p38/p38ALPHA | | |
| MMP1 | CLG/CLGN | NM_002421 | Matrix metallopeptidase 1 (interstitial |
| | | | collagenase) |
| NADSYN | FLJ10631/FLJ36703/FLJ40627 | NM_018161 | NAD synthetase 1 |
| 1 | | | |
| NEIL1 | FLJ22402/FPG1/NEI1/hFPG1 | NM_024608 | Nei endonuclease VIII-like 1 (E. coli) |
| NFKB1 | DKFZp686C01211/EBP- | NM_003998 | Nuclear factor of kappa light polypeptide gene |
| | 1/KBF1/MGC54151/NF-kappa-B/NFKB- | | enhancer in B-cells 1 |
| | p105/NFKB-p50/p105/p50 | | |
| NOS1 | IHPS1/NOS/nNOS | NM_000620 | Nitric oxide synthase 1 (neuronal) |
| NOS2 | HEP-NOS/INOS/NOS/NOS2A | NM_000625 | Nitric oxide synthase 2, inducible |
| NOS3 | ECNOS/eNOS | NM_000603 | Nitric oxide synthase 3 (endothelial cell) |
| PARP1 | ADPRT/ADPRT1/PARP/PARP- | NM_001618 | Poly (ADP-ribose) polymerase 1 |
| | 1/PPOL/pADPRT-1 | | |
| PARP2 | ADPRT2/ADPRTL2/ADPRTL3/PARP- | NM_005484 | Poly (ADP-ribose) polymerase 2 |
| | 2/pADPRT-2 | | |
| PARP3 | ADPRT3/ADPRTL2/ADPRTL3/IRT1/PAD | NM_005485 | Poly (ADP-ribose) polymerase family, member 3 |
| | PRT-3 | | |
| PARP4 | ADPRTL1/PARPL/PH5P/VAULT3/VPARP/ | NM_006437 | Poly (ADP-ribose) polymerase family, member 4 |
| | VWA5C/p193 | | |
| PARP9 | BAL/BAL1/DKFZp666B0810/DKFZp686M | NM_031458 | Poly (ADP-ribose) polymerase family, member 9 |
| | 15238/FLJ26637/FLJ35310/FLJ41418/FLJ4 | | |
| | 3593/MGC: 7868 | | |
| PDGFRL | PDGRL/PRLTS | NM_006207 | Platelet-derived growth factor receptor-like |
| POT1 | DKFZp586D211/hPot1 | NM_015450 | POT1 protection of telomeres 1 homolog |
| | | | (S. pombe) |
| PPARG | CIMT1/NR1C3/PPARG1/PPARG2/ | NM_015869 | Peroxisome proliferator-activated receptor |
| | PPARgamma | | gamma |
| PPARGC1 | LEM6/PGC-1(alpha)/PGC- | NM_013261 | Peroxisome proliferator-activated receptor |
| A | 1v/PGC1/PGC1A/PPARGC1 | | gamma, coactivator 1 alpha |
| PPC | PPC | SA_00103 | Positive PCR Control |
| PPC | PPC | SA_00103 | Positive PCR Control |
| PTGS2 | COX-2/COX2/GRIPGHS/PGG/HS/PGHS- | NM_000963 | Prostaglandin-endoperoxide synthase 2 |
| | 2/PHS-2/hCox-2 | | (prostaglandin G/H synthase and |
| | | | cyclooxygenase) |
| RAP1A | KREV-1/KREV1/RAP1/SMGP21 | NM_002884 | RAP1A, member of RAS oncogene family |
| RTC | RTC | SA_00104 | Reverse Transcription Control |
| RTC | RTC | SA_00104 | Reverse Transcription Control |
| S100A7 | PSOR1/S100A7c | NM_002963 | S100 calcium binding protein A7 |
| SERPINB | HsT1201/PAI/PAI-2/PAI2/PLANH2 | NM_002575 | Serpin peptidase inhibitor, clade B (ovalbumin), |
| 2 | | | member 2 |
| SHC1 | FLJ26504/SHC/SHCA | NM_003029 | SHC (Src homology 2 domain containing) |
| | | | transforming protein 1 |
| SIRT1 | SIR2L1 | NM_012238 | Sirtuin (silent mating type information regulation |
| | | | 2 homolog) 1 (S. cerevisiae) |
| SIRT2 | SIR2/SIR2L/SIR2L2 | NM_012237 | Sirtuin (silent mating type information regulation |
| | | | 2 homolog) 2 (S. cerevisiae) |
| SIRT3 | SIR2L3 | NM_012239 | Sirtuin (silent mating type information regulation |
| | | | 2 homolog) 3 (S. cerevisiae) |
| SIRT4 | MGC130046/MGC130047/MGC57437/SIR2 | NM_012240 | Sirtuin (silent mating type information regulation |
| | L4 | | 2 homolog) 4 (S. cerevisiae) |
| SOD1 | ALS/ALS1/IPOA/SOD/homodimer | NM_000454 | Superoxide dismutase 1, soluble |
| SOD2 | IPO-B/MNSOD/Mn-SOD | NM_000636 | Superoxide dismutase 2, mitochondrial |
| TEP1 | TLP1/TP1/TROVE1/VAULT2/p240 | NM_007110 | Telomerase-associated protein 1 |
| TERF2 | TRBF2/TRF2 | NM_005652 | Telomeric repeat binding factor 2 |
| TERT | EST2/TCS1/TP2/TRT/hEST2 | NM_198255 | Telomerase reverse transcriptase |
| TGFB1 | CED/DPD1/TGFB/TGFbeta | NM_000660 | Transforming growth factor, beta 1 |
| TIMM22 | TEX4/TIM22 | NM_013337 | Translocase of inner mitochondrial membrane 22 |
| | | | homolog (yeast) |
| TIMP3 | HSMRK222/K222/K222TA2/SFD | NM_000362 | TIMP metallopeptidase inhibitor 3 |
| TINF2 | TIN2/TIN2L | NM_012461 | TERF1 (TRF1)-interacting nuclear factor 2 |
| TNF | DIF/TNF-alpha/TNFA/TNFSF2 | NM_000594 | Tumor necrosis factor (TNF superfamily, |
| | | | member 2) |
| TOMM40 | C19orf1/D19S1177E/PER- | NM_006114 | Translocase of outer mitochondrial membrane 40 |
| | EC1/PEREC1/TOM40 | | homolog (yeast) |
| TP53 | FLJ92943/LFS1/TRP53/p53 | NM_000546 | Tumor protein p53 |
| TPP1 | CLN2/GIG1/LPIC/MGC21297 | NM_000391 | Tripeptidyl peptidase I |
| UBE2S | E2-EPF/E2EPF/EPF5 | NM_014501 | Ubiquitin-conjugating enzyme E2S |
| VEGFA | MGC70609/MVCD1/VEGF/VEGF-A/VPF | NM_003376 | Vascular endothelial growth factor A |

### Kits

Also provided herein are kits, e.g., kits for therapeutic purposes or kits for modulating the lifespan of cells or modulating apoptosis. A kit may comprise one or more activating or inhibitory compounds described herein, e.g., in premeasured doses. A kit may optionally comprise devices for contacting cells with the compounds and instructions for use. Devices include syringes, and other devices for introducing a compound into a subject or applying it to the skin of a subject.

Kits are provided which contain the necessary reagents for determining the level of expression of one or more genes (or the proteins encoded thereby) associated with longevity, mitochondrial biogenesis or health, and/or telomere or DNA repair or maintenance. Provided herein are lists and sets of genes the detection (and/or quantitation) of expression of which can be accomplished using kits. Instructions provided in the kits can include calibration curves, diagrams, illustrations, or charts or the like to compare with the determined (e.g., experimentally measured) values or other results.

A. Kits for Detection of mRNA Expression

Kits can be used to detect mRNA expression levels. Such kits may include an appropriate amount of one or more of the oligonucleotide primers for use in reverse transcription amplification reactions, similarly to those provided above, with art-obvious modifications for use with RNA.

In some embodiments, kits for detection of mRNA expression levels may also include the reagents necessary to carry out RT-PCR in vitro amplification reactions, including, for instance, RNA sample preparation reagents (including e.g., an RNAse inhibitor), appropriate buffers (e.g., polymerase buffer), salts (e.g., magnesium chloride), and deoxyribonucleotides (dNTPs). Written instructions may also be included.

Kits in addition may include either labeled or unlabeled oligonucleotide probes for use in detection of the in vitro amplified target sequences. The appropriate sequences for such a probe will be any sequence that falls between the annealing sites of the two provided oligonucleotide primers, such that the sequence the probe is complementary to is amplified during the PCR reaction.

It also may be advantageous to provided in the kit one or more control sequences for use in the RT-PCR reactions. The design of appropriate positive control sequences is well known to one of ordinary skill in the appropriate art.

Alternatively, kits may be provided with the necessary reagents to carry out quantitative or semi-quantitative Northern analysis of mRNA. Such kits include, for instance, at least one target sequence-specific oligonucleotide for use as a probe. This oligonucleotide may be labeled in any conventional way, including with a selected radioactive isotope, enzyme substrate, co-factor, ligand, chemiluminescent or fluorescent agent, hapten, or enzyme.

Also contemplated are kits containing an array, where the feature(s) of the array correspond to genes identified herein as associated with lifespan, longevity, mitochondrial health/maintenance/biogenesis, and/or telomere or DNA health or maintenance.

B. Kits for Detection of Protein or Peptide Expression

Kits for the detection of protein expression, include for instance at least one target protein specific binding agent (e.g., a polyclonal or monoclonal antibody or antibody fragment) for each protein target to be detected, and may include at least one control. The protein specific binding agent and control may be contained in separate containers. The kits may also include means for detecting target:agent complexes, for instance the agent may be detectably labeled. If the detectable agent is not labeled, it may be detected by second antibodies or protein A for example which may also be provided in some kits in one or more separate containers. Such techniques are well known.

Additional components in some kits include instructions for carrying out the assay. Instructions will allow the tester to determine whether protein expression levels are altered, for instance in comparison to a control sample. Reaction vessels and auxiliary reagents such as chromogens, buffers, enzymes, etc. may also be included in the kits.

By way of example only, an effective and convenient immunoassay kit such as an enzyme-linked immunosorbent assay can be constructed to test anti-target protein antibody in human serum. Expression vectors can be constructed using a human target cDNA to produce the recombinant human target protein in either bacteria or baculovirus. By affinity purification, unlimited amounts of pure recombinant protein can be produced.

Assay kits in some embodiments provide the recombinant protein(s) as an antigen and enzyme-conjugated e.g., goat anti-human IgG as a second antibody as well as enzymatic substrates. Such kits can be used to test if a subject's serum contains antibodies against a target lifespan extension associated protein (or a collection of them).

The present description is further illustrated by the following examples, which should not be construed as limiting in any way. The contents of all cited references (including literature references, issued patents, and published patent applications as cited throughout this application) are hereby expressly incorporated by reference. Any publicly available sequences referenced herein are incorporated by reference from the public database as they were available on Dec. 1, 2008 (the date of filing of the first priority application).

### EXAMPLES

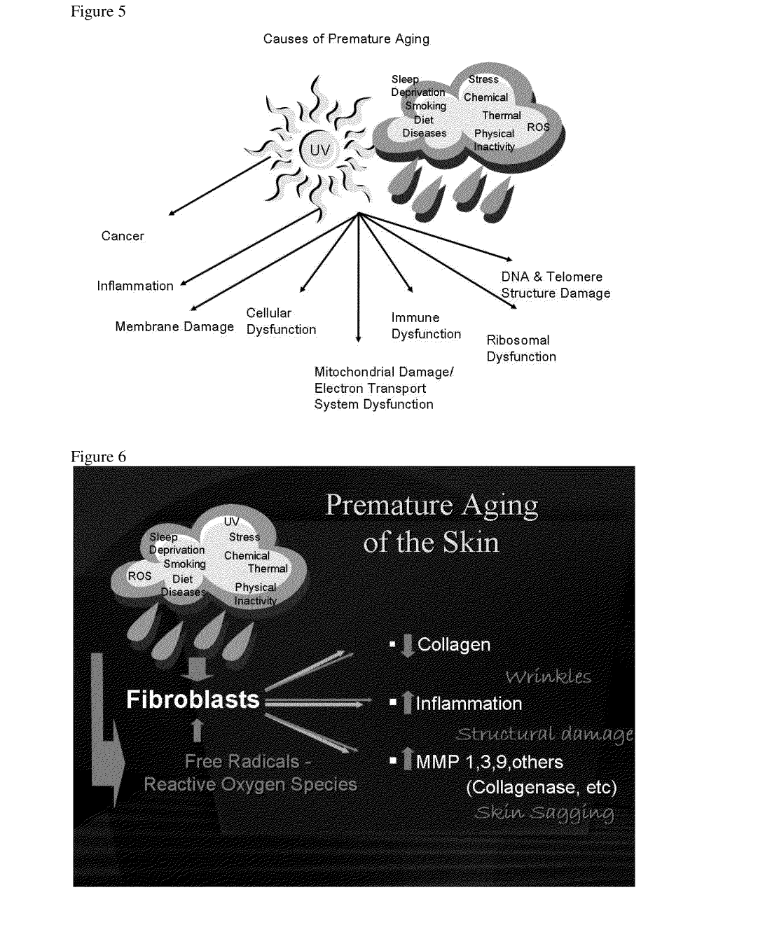

The effects of oxidative stress, environmental damage and premature aging are almost as diverse as their causative agents (FIGS. 4, 5, and 6). The mechanistic pathways most commonly affecting premature aging and anti-Longevity effects involve the AP1 matrix regulation pathway, mitochondrial DNA damage/deletions, telomere shortening, inflammation, and cancer cell creation. These pathways are affected through environmental damage in the form(s) of UV radiation (all types and full spectrum), thermal injury, chronic or acute disease states/conditions, smoking, chemicals, dietary habits, and oxidative stress/free radical formation. All of these effects can modulate the cells gene responses, mitochondrial numbers and/or efficiency, and elimination of ROS from the cellular environment in a negative fashion, much like antioxidants and the other described compounds can modulate the same responses in the direction of increased lifespan.

The following experimental examples illustrate the use of UV radiation to affect the negative or lifespan shortening affects previously described. UV radiation was selected as an injury producing agent for several reasons: 1) it is a classic model used in the literature to injure cells in culture, 2) its dosage is easily controlled and directed, and 3) it is one of the most ubiquitous sources of environmental injury that cells and tissues will face on a daily basis in real world settings. The use of UV and H₂O₂ is by no means meant to limit the application or interpretation of these results, and are meant to serve as examples of the type of pro-longevity modulation that can be achieved through proper application of the described compounds. Any method of the previously recognized environmental agents (oxidative stress, thermal injury, smoking, hypoxia, and so forth) could have been and may be used in the future expansion of these experimental examples that follow.

### Example 1

This example illustrates protection of telomere length maintenance and/or lifespan extension through application of modulating compounds (exemplified by green tea polyphenols).

Cell cultures: Two human skin fibroblast cell cultures were obtained through the Coriell Cell Repository from the National Institute on Aging Cell Repository. The cultures were established from biopsies of a Caucasian female, at 36 years and again at 50 years of age (AG7308 and AG14271 respectively).

Culture media: Cells were grown in Minimal Essential Medium supplemented with 10% fetal bovine serum, 2 mM L-glutamine, 2 mM Glutamax I, and 1× MEM non-essential amino acids solution. Cells were washed in the same medium, but without the fetal bovine serum. During the 24 hour experimental phase, cells were maintained in the same medium, but with only 1% fetal bovine serum. All cultures were incubated at 37° C. with 5% CO₂ in a humidified chamber.

Seeding of cells: On Day 1, each cell culture was seeded into 6 well cluster dishes at ˜1.5×10⁵ cells in 4 mls medium per well. Three wells were seeded per test condition or control.

Experimental phase: On Day 3, wells were washed 1× with 2 mls medium, fed back 2 mls medium without fetal bovine serum and preincubated for 30-60 minutes before challenge with test or control conditions. After the preincubation, medium was aspirated from the wells and 2.5 mls of test or control condition in medium with 1% fetal bovine serum was pipetted into the appropriate wells.

The green tea polyphenols used were Premier Green Tea Extract Lot #10783, obtained from Pharma Cosmetix Research, LLC (Richmond, Va.). The Green Tea extract was measured into a stock solution of the above described Minimal Essential Media+1% Fetal Bovine Serum in a w/v ratio and then serially diluted into the testing concentrations with MEM+1% Fetal Bovine Serum.

Three conditions were tested: (1) cells were exposed to the test condition 4 hours before UVB exposure, but not during or after UVB exposure (2) cells were exposed to the test condition after UVB exposure, but not before or during (3) cells were exposed to the test condition 4 hours before, then during and after UVB exposure. UVB exposed and unexposed cells without test condition were used as controls. The experimental phase ended at 24 hours post UVB exposure. Cells from each test and control parameter were then trypsinized, collected by centrifugation, washed 3 times in PBS, and a cell count made using a hemacytometer. The final pellet was stored at −80° C. until processed further.

UVB exposure: Cells exposed to UVB received 200 mJ/cm² UVB using ThermoOriel solar simulator model SP66923-3056. Cells were exposed from the bottom of the culture dish. The UVB dose delivered to the cells was adjusted for interference from the plastic in the culture dish.

Preparation of cell extract: Extract from the cell pellets stored at −80 C post experimental phase was prepared for PCR according to the instructions in the Allied Biotech, Inc, Quantitative Telomerase Detection Kit. Briefly, pellets were thawed and immediately resuspended in 200 ul 1× Lysis Buffer per 10⁻⁵ to 10⁻⁶ cells. The suspension was incubated on ice for 30 minutes, then microcentrifuged at 12,000×g for 30 minutes at 4° C. The supernatant was aliquoted and stored at −80° C.

Detection of Telomerase: The extract from the cells allows for determination of telomerase activity by coupling the extract's ability to form telomeric repeats onto an oligonucleotide substrate and the resultant extended product are amplified using Polymerase Chain Reaction. These products are then visualized with SYBR green a fluorescent detection agent that emits green fluorescence when bound to the double stranded DNA product. Each 25 μl PCR assay included 12.5 μl of 2×QTD Premix, 1.0 μl of Cell Extract, and 11.5 μl of Molecular Grade™ H₂O (distilled, deionized, sterile-filtered water, which is ultrapure and DNase, RNase and protease-free).

The samples were run in a BioRad iCycler for 20 minutes at 25° C. to complete the telomerase reaction. The PCR initial activation step followed immediately and was of 10 minute duration at 60° C. The iCycler then ran the denaturing, annealing, and extension (30 seconds at 95, 60 and 72° C. respectively) series for 40 cycles. The SYBR green detection occurred during the extension phase.

Telomerase Detection Results: The results for N=3 were downloaded from the iCycler into a modified array analysis program and the results examined for statistical significance. The raw analysis is provided in DATA TABLE 1.

DATA TABLE 1
(Level of telomerase -- ability to form telomeric repeats onto
an oligonucleotide substrate; treated with Green Tea Extracts)

| | p | fold |
|---|---|---|
| | value | change |
| Compared to UnTx Control (36 yo) | | |
| 36 yo UVB Alone | 0.1515 | −1.43 |
| 36 yo UVB + gren tea Continuous Exposure | 0.1770 | −1.39 |
| 36 yo UVB + green tea After | 0.1550 | 1.29 |
| 36 yo UVB + green tea Before | 0.0634 | −1.49 |
| 36 yo Untreated | 1.0000 | −1.00 |
| 50 yo UVB Alone | 0.4305 | −1.78 |
| 50 yo UVB + green tea Continuous Exposure | 0.5156 | −1.15 |
| 50 yo UVB + green tea After | 0.8192 | 1.04 |
| 50 yo UVB + green tea Before | 0.3690 | −6.22 |
| 50 yo Untreated | 0.2718 | −1.30 |
| Compared to UnTx Control (50 yo) | | |
| 36 yo UVB Alone | 0.6966 | −1.10 |
| 36 yo UVB + green tea Continuous Exposure | 0.7937 | −1.06 |
| 36 yo UVB + green tea After | 0.0411 | 1.68 |
| 36 yo UVB + green tea Before | 0.5086 | −1.14 |
| 36 yo Untreated | 0.2718 | 1.30 |
| Compared too UnTx Control (50 yo) | | |
| 50 yo UVB Alone | 0.6642 | −1.37 |
| 50 yo UVB + green tea Continuous Exposure | 0.5909 | 1.14 |
| 50 yo UVB + green tea After | 0.1686 | 1.35 |
| 50 yo UVB + green tea Before | 0.4367 | −4.77 |
| 50 yo Untreated | 1.0000 | −1.00 |
| Compared to UVB Tx Control (36 yo) | | |
| 36 yo UVB Alone | 1.0000 | −1.00 |
| 36 yo UVB + green tea Continuous Exposure | 0.8894 | 1.03 |
| 36 yo UVB + green tea After | 0.0220 | 1.84 |
| 36 yo UVB + green tea Before | 0.8372 | −1.04 |
| 36 yo Untreated | 0.1515 | 1.43 |
| 50 yo UVB Alone | 0.7604 | −1.24 |
| 50 yo UVB + green tea Continuous Exposure | 0.3566 | 1.25 |
| 50 yo UVB + green tea After | 0.0851 | 1.49 |
| 50 yo UVB + green tea Before | 0.4628 | −4.34 |
| 50 yo Untreated | 0.6966 | 1.10 |
| Compared to UVB Tx Control (50 yo) | | |
| 36 yo UVB Alone | 0.7604 | 1.24 |
| 36 yo UVB + green tea Continuous Exposure | 0.7262 | 1.28 |
| 36 yo UVB + green tea After | 0.2705 | 2.29 |
| 36 yo UVB + green tea Before | 0.7985 | 1.19 |
| 36 yo Untreated | 0.4305 | 1.78 |
| 50 yo UVB Alone | 1.0000 | −1.00 |
| 50 yo UVB + green tea Continuous Exposure | 0.5437 | 1.55 |
| 50 yo UVB + green tea After | 0.3986 | 1.85 |
| 50 yo UVB + green tea Before | 0.5495 | −3.49 |
| 50 yo Untreated | 0.6642 | 1.37 |
| 36 yo Vs. 50 yo Untreated | 0.2718 | 1.30 |
| 36 yo green tea Before Vs. green tea After | 0.0037 | −1.92 |
| 50 yo green tea Before Vs. green tea After | 0.3597 | −6.45 |
| 36 yo UVB + green tea Cont Vs. 36 yo green tea After | 0.0243 | −1.79 |
| 50 yo UVB + green tea Cont Vs. 50 yo green tea After | 0.3443 | −1.19 |
| 36 yo UVB + green tea Cont Vs. 36 yo green tea | 0.7042 | 1.07 |
| Before | | |
| 50 yo UVB + green tea Cont Vs. 50 yo green tea | 0.4031 | 5.41 |
| Before | | |
| 36 yo UVB + green tea Cont Vs. 50 yo green tea | 0.4537 | 4.48 |
| Before | | |
| 50 yo UVB + green tea Cont Vs. 36 yo green tea | 0.1969 | 1.30 |
| Before | | |
| 36 yo UVB + green tea Cont Vs. 50 yo green tea After | 0.0995 | −1.44 |
| 50 yo UVB + green tea Cont Vs. 36 yo green tea After | 0.0678 | −1.48 |
| 36 yo green tea Before Vs. 50 yo green tea After | 0.4730 | 4.17 |
| 50 yo green tea Before Vs. 36 yo green tea After | 0.3131 | −8.00 |

### Example 2

This example illustrates protection of telomere length maintenance and/or lifespan extension through application of modulating compounds (idebenone and coffee cherry).

Cell cultures: Two human skin fibroblast cell cultures were obtained through the Coriell Cell Repository from the National Institute on Aging Cell Repository. The cultures were established from biopsies of a Caucasian female, at 36 years and again at 50 years of age, AG7308 and AG14271 respectively.

Culture media: Cells were grown in Minimal Essential Medium supplemented with 10% fetal bovine serum, 2 mM L-glutamine, 2 mM Glutamax I, and 1×MEM non-essential amino acids solution. Cells were washed in the same medium, but without the fetal bovine serum. During the 24 hour experimental phase, cells were maintained in the same medium, but with only 1% fetal bovine serum. All cultures were incubated at 37° C. with 5% CO₂ in a humidified chamber.

Seeding of cells: On Day 1, each cell culture was seeded into 6 well cluster dishes at ˜1.5×10⁵ cells in 4 mls medium per well. Three wells were seeded per test condition or control.

Experimental phase: On Day 3, wells were washed 1× with 2 mls medium, fed back 2 mls medium without fetal bovine serum and preincubated for 30-60 minutes before challenge with test or control conditions. After the preincubation, medium was aspirated from the wells and 2.5 mls of test or control condition in medium with 1% fetal bovine serum was pipetted into the appropriate wells.

The coffee cherry Beauty extract (COFFEEBERRY®; VDF FutureCeuticals, Inc., Momence, Ill.) was placed into a stock solution of 1% coffee cherry (w/v) in the previously described Minimal Essential Medium+1% Fetal Bovine Serum and DMSO. This stock solution was diluted in a 1:10 ratio with the MEM+1% FBS previously described until the testing concentrations were reached. At the testing concentrations, the DMSO content was less than 0.01%— well within safe limits for tissue culture. Idebenone dilutions were prepared in a similar fashion with the stock 1% solution being dissolved in sterile alcohol. The 1% stock solution was also diluted with MEM+1% FBS until the testing concentrations were reached.

Three conditions were tested: (1) cells were exposed to the test condition 4 hours before UVB exposure, but not during or after UVB exposure (2) cells were exposed to the test condition after UVB exposure, but not before or during (3) cells were exposed to the test condition 4 hours before, then during and after UVB exposure. UVB exposed and unexposed cells without test condition were used as controls. The experimental phase ended at 24 hours post UVB exposure. Cells from each test and control parameter were then trypsinized, collected by centrifugation, washed 3 times in PBS, and a cell count made using a hemacytometer. The final pellet was stored at −80° C. until processed further.

UVB exposure: Cells exposed to UVB received 200 mJ/cm² UVB using ThermoOriel solar simulator model SP66923-3056. Cells were exposed from the bottom of the culture dish. The UVB dose delivered to the cells was adjusted for interference from the plastic in the culture dish.

Preparation of cell extract: Extract from the cell pellets stored at −80 C post experimental phase was prepared for PCR according to the instructions in the Allied Biotech, Inc, Quantitative Telomerase Detection Kit. Briefly, pellets were thawed and immediately resuspended in 200 ul 1× Lysis Buffer per 10⁻⁵ to 10⁻⁶ cells. The suspension was incubated on ice for 30 minutes, then microcentrifuged at 12,000×g for 30 minutes at 4° C. The supernatant was aliquoted and stored at −80° C.

Detection of Telomerase: The extract from the cells allows for determination of telomerase activity by coupling the extract's ability to form telomeric repeats onto an oligonucleotide substrate and the resultant extended product are amplified using Polymerase Chain Reaction. These products are then visualized with SYBR green a fluorescent detection agent that emits green fluorescence when bound to the double stranded DNA product. Each 25 μl PCR assay included 12.5 μl of 2×QTD Premix, 1.0 μl of Cell Extract, and 11.5 μl of Molecular Grade H2O.

The samples were run in a BioRad iCycler for 20 minutes at 25° C. to complete the telomerase reaction. The PCR initial activation step followed immediately and was of 10 minute duration at 60° C. The iCycler then ran the denaturing, annealing, and extension (30 seconds at 95, 60 and 72° C. respectively) series for 40 cycles. The SYBR green detection occurred during the extension phase.

Telomerase Detection Results: The results for N=3 were downloaded from the iCycler into a modified array analysis program and the results examined for statistical significance. The raw analysis is provided in DATA TABLE 2.

DATA TABLE 2
(Level of telomerase -- ability to form telomeric
repeats onto an oligonucleotide substrate; treated
with idebenone & Coffee cherry extracts)

| Compared to UnTx Control | | |
|---|---|---|
| 36 yo UVB Alone | 0.0001 | 97.01 |
| 36 yo UVB + idebenone 4 hr | 0.0001 | 90.51 |
| 36 yo UVB + idebenone Continuous | 0.0001 | 97.01 |
| 36 yo UVB + coffee cherry 4 hr | 0.0001 | 78.79 |
| 36 yo UVB + coffee cherry Continuous | 0.0002 | 101.59 |
| Compared to UVB Tx Control | | |
| 36 yo UVB Alone | 1.0000 | −1.00 |
| 36 yo UVB + idebenone 4 hr | 0.7625 | −1.07 |
| 36 yo UVB + idebenone Continuous | 1.0000 | −1.00 |
| 36 yo UVB + coffee cherry 4 hr | 0.3712 | −1.23 |
| 36 yo UVB + coffee cherry Continuous | 0.8971 | 1.05 |
| 36 yo idebenone Vs. 50 yo idebenone 4 hr | 0.2102 | 1.46 |
| 36 yo UVA Vs. 36 yo UVB | 0.5631 | −1.19 |
| 50 yo UVA Vs. 50 yo UVB | N/A | −111.43 |
| 36 yo Untx Vs. 36 yo UVA | 0.6440 | −1.20 |
| 36 yo UVA Vs. 50 yo UVA | 0.0532 | 1.68 |

Conclusions (with Good p Values/Statistical Significance):

36 year old cells (that is, cells from a 36 year old person) given green tea have a higher level of telomerase activity than 50 year old cells given the same dose of green tea (+1.33 fold or 33% increase in telomerase activity)

36 year old cells given green tea before being stressed with 1 MED of UVB have a +2.8 fold (roughly 180%) increase in telomerase activity when compared to the same cells given Idebenone.

50 year old cells given green tea before being stressed with 1 MED of UVB have a +3.36 fold (roughly 236%) increase in telomerase activity when compared to the same cells given Idebenone.

36 year old cells given Idebenone have roughly 53% less activity than untreated cells.

36 year old cells given Idebenone and stressed with 1 MED UVB have −2.4 fold less activity than untreated cells, and −1.7 fold less activity than UVB stressed cells alone.

50 year old cells given Idebenone and stressed with 1 MED of UVB have roughly −2.61 fold decrease in telomerase activity when compared to untreated controls, and −2.65 fold less when compared to UVB treated age matched controls.

There is a slight increase in telomerase activity in 36 y.o. cells treated with green tea alone and UVB stressed+green tea when compared to UVB treated cells. (+1.55 and +1.65 respectively)

When UVB unstressed 36 y.o. cells are given green tea there is an increase of telomerase activity (+1.69 fold or 69%) compared to age matched unstressed cells receiving Idebenone.

A final trend with lower p value and statistical significance shows that telomerase activity is lower in 36 year old untreated cells than in 50 year old untreated cells.

### Example 3

This example describes examination of the gene expression profile related to aging, lifespan and telomerase length maintenance of cells contacted with coffee cherry extract and idebenone demonstrate lifespan and/or telomere length extension characteristics.

Cell cultures: Two human skin fibroblast cell cultures were obtained through the Coriell Cell Repository from the National Institute on Aging Cell Repository. The cultures were established from biopsies of a Caucasian female, at 36 years and again at 50 years of age, AG7308 and AG14271 respectively.

Culture media: Cells were grown in Minimal Essential Medium supplemented with 10% fetal bovine serum, 2 mM L-glutamine, 2 mM Glutamax I, and 1×MEM non-essential amino acids solution. Cells were washed in the same medium, but without the fetal bovine serum. During the 24 hour experimental phase, cells were maintained in the same medium, but with only 1% fetal bovine serum. All cultures were incubated at 37° C. with 5% CO₂ in a humidified chamber.

Seeding of cells: On Day 1, each cell culture was seeded into 6 well cluster dishes at ˜1.5×10⁵ cells in 4 mls medium per well. Three wells were seeded per test condition or control.

Experimental phase: On Day 3, wells were washed 1× with 2 mls medium, fed back 2 mls medium without fetal bovine serum and preincubated for 30-60 minutes before challenge with test or control conditions. After the preincubation, medium was aspirated from the wells and 2.5 mls of test or control condition in medium with 1% fetal bovine serum was pipetted into the appropriate wells.

Nine conditions were tested: (1) cells were exposed to one of the test conditions (1 μM Idebenone, 0.001% coffee cherry, or 0.001% green tea extract; sourced and prepared as described above) 4 hours before UV exposure, but not during or after UV exposure (2) cells were exposed to one of the test conditions 4 hours before, then during and after UV exposure. (3) UVA1, UVB exposed and unexposed cells without test condition were used as controls. The experimental phase ended at 24 hours post UV exposure. Cells from each test and control parameter were then prepped for RNA isolation as described below.

UVB/UVA1 exposure: Cells exposed to UV received 200 mJ/cm² UVB/1 MED UVA1 using Thermo Oriel solar simulator model SP66923-3056. Cells were exposed from the bottom of the culture dish. The UV dose delivered to the cells was adjusted for interference from the plastic in the culture dish.

Preparation of RNA: The RNA was isolated using the Qiagen RNEasy Plus Mini Kit according to the manufacturer's protocol which can be found on the World Wilde Web at qiagen.com/literature/Defaultaspx?Term=&Language=EN&LiteratureType=4& ProductCategory=10162. The RNA was quantified and examined for purity using the 260/280 nm read method in a μ Quant spectrophotometer before use in the array.

Creation and Performance of a Custom Microarray (“Array 1”): Through the Custom Array design service of Superarray Bioscience Corporation, a 96 well RT-PCR microarray (“Array 1”) was developed to illustrate genetic responses in cells treated as above for specific genes involved in longevity as well as the relevant quality controls. The array was performed in compliance with manufacturer's guidelines and by recommended manufacturer's protocol as can be found on the World Wilde Web at superarray.com/Manual/perarrayplate.pdf

Custom Microarray Results: The results from the microarrays were determined using the delta CT method which uses comparisons of control genes and threshold cycles of genes of interest to generate relative expression values. The full set of experimental conditions was cross referenced and the data is provided herewith in DATA TABLE 3.

### Example 4

This example describes examination of the gene expression profile related to aging, lifespan and telomerase length maintenance of cells contacted with green tea polyphenols and idebenone demonstrate lifespan and/or telomere length extension characteristics.

Cell cultures: Two human skin fibroblast cell cultures were obtained through the Coriell Cell Repository from the National Institute on Aging Cell Repository. The cultures were established from biopsies of a Caucasian female, at 36 years and again at 50 years of age, AG7308 and AG14271 respectively.

Culture media: Cells were grown in Minimal Essential Medium supplemented with 10% fetal bovine serum, 2 mM L-glutamine, 2 mM Glutamax I, and 1×MEM non-essential amino acids solution. Cells were washed in the same medium, but without the fetal bovine serum. During the 24 hour experimental phase, cells were maintained in the same medium, but with only 1% fetal bovine serum. All cultures were incubated at 37 C with 5% CO₂ in a humidified chamber.

Seeding of cells: On Day 1, each cell culture was seeded into 6 well cluster dishes at ˜1.5×10⁵ cells in 4 mls medium per well. Three wells were seeded per test condition or control.

Experimental phase: On Day 3, wells were washed 1× with 2 mls medium, fed back 2 mls medium without fetal bovine serum and preincubated for 30-60 minutes before challenge with test or control conditions. After the preincubation, medium was aspirated from the wells and 2.5 mls of test or control condition in medium with 1% fetal bovine serum was pipetted into the appropriate wells.

Nine conditions were tested: (1) cells were exposed to one of the test conditions (1 μM Idebenone, 0.001% coffee cherry, or 0.001% Green tea) 4 hours before UV exposure, but not during or after UV exposure (2) cells were exposed to one of the test conditions 4 hours before, then during and after UV exposure. (3) UVA1, UVB exposed and unexposed cells without test condition were used as controls. The experimental phase ended at 24 hours post UV exposure. Cells from each test and control parameter were then prepped for RNA isolation as described below.

UVB/UVA1 exposure: Cells exposed to UV received 200 mJ/cm² UVB/1 MED UVA1 using Thermo Oriel solar simulator model SP66923-3056. Cells were exposed from the bottom of the culture dish. The UV dose delivered to the cells was adjusted for interference from the plastic in the culture dish.

Preparation of RNA: The RNA was isolated using the Qiagen RNEasy Plus Mini Kit according to the manufacturer's protocol which can be found on the World Wide Web at qiagen.com/literature/Default. aspx?Term=&Language=EN&LiteratureType=4&ProductCategory=10162.

The RNA was quantified and examined for purity using the 260/280 nm read method in a μ Quant spectrophotometer before use in the array.

Creation and Performance of the Custom Microarray: Through the Custom Array design service of Superarray Bioscience Corporation, a 96 well RT-PCR Microarray was developed to illustrate genetic responses in cells treated as above for specific genes involved in longevity as well as the relevant quality controls. The array was performed in compliance with manufacturer's guidelines and by recommended manufacturer's protocol as can be found on the World Wide Web at superarray.com/Manual/perarrayplate.pdf.

Custom Microarray Results: The results from the microarrays were determined using the delta CT method which uses comparisons of control genes and threshold cycles of genes of interest to generate relative expression values. The full set of experimental conditions was cross referenced and the data is provided herewith in DATA TABLE 4.

### Example 5

This example describes examination of the gene expression profile related to aging, lifespan and telomerase length maintenance of cells contacted with green tea polyphenols and coffee cherry extract demonstrate lifespan and/or telomere length extension characteristics.

Cell cultures: Two human skin fibroblast cell cultures were obtained through the Coriell Cell Repository from the National Institute on Aging Cell Repository. The cultures were established from biopsies of a Caucasian female, at 36 years and again at 50 years of age, AG7308 and AG14271 respectively.

Culture media: Cells were grown in Minimal Essential Medium supplemented with 10% fetal bovine serum, 2 mM L-glutamine, 2 mM Glutamax I, and 1×MEM non-essential amino acids solution. Cells were washed in the same medium, but without the fetal bovine serum. During the 24 hour experimental phase, cells were maintained in the same medium, but with only 1% fetal bovine serum. All cultures were incubated at 37° C. with 5% CO₂ in a humidified chamber.

Seeding of cells: On Day 1, each cell culture was seeded into 6 well cluster dishes at ˜1.5×10⁵ cells in 4 mls medium per well. Three wells were seeded per test condition or control.

Experimental phase: On Day 3, wells were washed 1× with 2 mls medium, fed back 2 mls medium without fetal bovine serum and preincubated for 30-60 minutes before challenge with test or control conditions. After the preincubation, medium was aspirated from the wells and 2.5 mls of test or control condition in medium with 1% fetal bovine serum was pipetted into the appropriate wells.

Nine conditions were tested: (1) cells were exposed to one of the test conditions (1 μM Idebenone, 0.001% coffee cherry, or 0.001% Green tea) 4 hours before UV exposure, but not during or after UV exposure (2) cells were exposed to one of the test conditions 4 hours before, then during and after UV exposure. (3) UVA1, UVB exposed and unexposed cells without test condition were used as controls. The experimental phase ended at 24 hours post UV exposure. Cells from each test and control parameter were then prepped for RNA isolation as described below.

UVB/UVA1 exposure: Cells exposed to UV received 200 mJ/cm² UVB/1 MED UVA1 using Thermo Oriel solar simulator model SP66923-3056. Cells were exposed from the bottom of the culture dish. The UV dose delivered to the cells was adjusted for interference from the plastic in the culture dish.

Preparation of RNA: The RNA was isolated using the Qiagen RNEasy Plus Mini Kit according to the manufacturer's protocol which can be found on the World Wide Web at qiagen.com/literature/Default. aspx?Term=&Language=EN&LiteratureType=4&ProductCategory=10162. The RNA was quantified and examined for purity using the 260/280 nm read method in a μQuant spectrophotometer before use in the array.

Creation and Performance of the Custom Microarray: Through the Custom Array design service of Superarray Bioscience Corporation, a 96 well RT-PCR Microarray was developed to illustrate genetic responses in cells treated as above for specific genes involved in longevity as well as the relevant quality controls. The array was performed in compliance with manufacturer's guidelines and by recommended manufacturer's protocol as can be found on the World Wide Web at superarray.com/Manual/perarrayplate.pdf.

Custom Microarray Results: The results from the microarrays were determined using the delta CT method which uses comparisons of control genes and threshold cycles of genes of interest to generate relative expression values. The full set of experimental conditions was cross referenced and the data is provided herewith in DATA TABLE 5.

### Example 6

This example describes application of UVA/UVB injury to cells, which demonstrates a decrease in lifespan or telomere length maintenance gene expression profiles.

Cell cultures: Two human skin fibroblast cell cultures were obtained through the Coriell Cell Repository from the National Institute on Aging Cell Repository. The cultures were established from biopsies of a Caucasian female, at 36 years and again at 50 years of age, AG7308 and AG14271 respectively.

Culture media: Cells were grown in Minimal Essential Medium supplemented with 10% fetal bovine serum, 2 mM L-glutamine, 2 mM Glutamax I, and 1×MEM non-essential amino acids solution. Cells were washed in the same medium, but without the fetal bovine serum. During the 24 hour experimental phase, cells were maintained in the same medium, but with only 1% fetal bovine serum. All cultures were incubated at 37° C. with 5% CO₂ in a humidified chamber.

Seeding of cells: On Day 1, each cell culture was seeded into 6 well cluster dishes at ˜1.5×10⁵ cells in 4 mls medium per well. Three wells were seeded per test condition or control.

Experimental phase: On Day 3, wells were washed 1× with 2 mls medium, fed back 2 mls medium without fetal bovine serum and preincubated for 30-60 minutes before challenge with test or control conditions. After the preincubation, medium was aspirated from the wells and 2.5 mls of test or control condition in medium with 1% fetal bovine serum was pipetted into the appropriate wells.

Nine conditions were tested: (1) cells were exposed to one of the test conditions (1 μM Idebenone, 0.001% coffee cherry, or 0.001% Green tea) 4 hours before UV exposure, but not during or after UV exposure (2) cells were exposed to one of the test conditions 4 hours before, then during and after UV exposure. (3) UVA1, UVB exposed and unexposed cells without test condition were used as controls. The experimental phase ended at 24 hours post UV exposure. Cells from each test and control parameter were then prepped for RNA isolation as described below.

UVB/UVA1 exposure: Cells exposed to UV received 200 mJ/cm² UVB/1 MED UVA1 using Thermo Oriel solar simulator model SP66923-3056. Cells were exposed from the bottom of the culture dish. The UV dose delivered to the cells was adjusted for interference from the plastic in the culture dish.

Preparation of RNA: The RNA was isolated using the Qiagen RNEasy Plus Mini Kit according to the manufacturer's protocol which can be found on the World Wide Web at qiagen.com/literature/Default. aspx?Term=&Language=EN&LiteratureType=4&ProductCategory=10162. The RNA was quantified and examined for purity using the 260/280 nm read method in a μQuant spectrophotometer before use in the array.

Creation and Performance of the Custom Microarray: Through the Custom Array design service of Superarray Bioscience Corporation, a 96 well RT-PCR Microarray was developed to illustrate genetic responses in cells treated as above for specific genes involved in longevity as well as the relevant quality controls. The array was performed in compliance with manufacturer's guidelines and by recommended manufacturer's protocol as can be found on the World Wide Web at superarray.com/Manual/perarrayplate.pdf.

Custom Microarray Results: The results from the microarrays were determined using the delta CT method which uses comparisons of control genes and threshold cycles of genes of interest to generate relative expression values. The full set of experimental conditions was cross referenced and the data is provided herewith in DATA TABLE 6.

Summary of Results (Examples 3-6): An analysis of the results indicates:

The condition with the most statistically significant genes affected is the 50 year old cells that were incubated in coffee cherry continuously and exposed to UVB, with seven genes altered. The next highest significant gene total (with six genes altered) is a tie with 36 year old cells given Green tea before UVB exposure both continuously as well as 4 hr beforehand, and the 4 hr incubation of 36 year old cells and Idebenone. Both cell lines, when exposed only to the radiation types tested, showed a significant and minimum of 4 fold reduction in the TERT gene.

In the 36 year old cell line cells exposed to UVB demonstrated a significant reduction in PARP1, NADSYN1, IFI44, TERT and NFKB1. In the same 36 year old cells treated with the anti-oxidant compounds, those same genes demonstrate either no significant up or down regulation or are significantly up regulated. This altered gene expression pattern is encouraging that the anti-oxidant compounds provide some ability to “reverse or improve” the gene expression triggered by the UVB exposure.

| 36 year old | | Green tea | Green tea | Idebenone | Idebenone | Coffee cherry | Coffee cherry |
|---|---|---|---|---|---|---|---|
| cell line | UVB | (4 hr) | (Continuous) | (4 hr) | (Continuous) | (4 hr inc.) | (Cont. inc.) |
| PARP1 | −5.14 | 6.73 | 4.42 | 4.27 | N/A | N/A | 4.92 |
| NADSYN1 | −3.06 | 3.61 | 3.35 | 2.21 | N/A | 2.61 | 2.93 |
| IFI44 | −3.39 | 3.36 | 2.72 | 2.63 | 2.98 | N/A | 3.86 |
| TERT | −4.63 | N/A | 1.23 | 8.54 | 4.36 | 8.20 | N/A |
| NFKB1 | −2.32 | 3.36 | N/A | 3.24 | N/A | N/A | 2.73 |

In the 50 year old cell line, the above statement also holds true. Interestingly, the only gene that showed statistical significance in UVB exposed cells was TERT. It is downregulated and to a larger degree than in the 36 year old cells. Again treatment with anti-oxidants is either not significant or up regulates the TERT gene.

| | | Green tea | Green tea | | | | |
|---|---|---|---|---|---|---|---|
| 50 year old | | (4 hr | (Continuous | Idebenone | Idebenone | Coffee cherry | Coffee cherry |
| cell line | UVB | incubation) | inc.) | (4 hr inc.) | (Cont. inc.) | (4 hr inc.) | (Cont. inc.) |
| TERT | −7.01 | N/A | N/A | N/A | 8.50 | 10.14 | 8.97 |

PARP1, a gene significantly downregulated in UVB exposed 36 year old cells and upregulated in cells exposed to antioxidant compounds, may play an intriguing role and be worth further study. The gene itself is involved in DNA repair, apoptosis and maintenance of optimal niacin status in the skin.

Green tea continuous incubation+UVB treated 36 year old cells demonstrate a downregulation of TNF of −18.44. TNF is a pro-inflammatory cytokine that plays a pathogenic role in age related diseases.

50 year old cells treated with UVA1 alone show a significant downregulation in CYP19A1 of −10.5 fold. CYP19A1 is a variant of cytochrome P-450 and is involved in xenobiotic metabolism and detoxification.

### Example 7

This example illustrates that application of modulating compounds (green tea polyphenols, coffee cherry extract and idebenone) to cells demonstrates ability to alter the gene expression of key components of the telomere length maintenance complex.

Cell cultures: A human skin fibroblast cell culture was obtained through the Coriell Cell Repository from the National Institute on Aging Cell Repository. The culture, AG07999, was established from a biopsy of a 32 year old Caucasian female.

Culture media: Cells were grown in Minimal Essential Medium supplemented with 10% fetal bovine serum, 2 mM L-glutamine, 2 mM Glutamax I. During the 24 hour experimental phase, cells were maintained in the same medium, but with only 1% fetal bovine serum. All cultures were incubated at 37° C. with 5% CO₂ in a humidified chamber.

Seeding of cells: On Day 1, 1 ml of 5.0−6.5×10⁵ cells/ml was seeded into each of four 75 cm² flasks containing 20 ml of culture medium.

Experimental phase: On Day 4, the medium was removed by aspiration and replaced with the test condition in 20 ml culture medium but with only 1% fetal bovine serum. Test conditions (prepared and sourced as above) were 1) 1 uM idebenone 2) 0.001% coffee cherry 3) 0.001% green tea and 4) vehicle control. All four conditions had a final vehicle concentration of 0.01% DMSO. On Day 5 after 24 hours of exposure to test conditions, the cells were isolated and the RNA isolated using the RT²q PCR-grade RNA isolation kit (Superarray Bioscience Corporation).

Preparation of RNA: The RNA was isolated using the RT²q PCR-grade RNA isolation kit according to the manufacturer's protocol which can be found on the World Wide Web at superarray.com/Manual/qpcgrade.pdf.

The RNA was quantified and examined for purity using the 260/280 nm read method in a μQuant spectrophotometer before use in the array.

Performance of the qPCR Primer Assays: Assays for six exemplary genes of interest (TERT, TPP1, TERF1, TERF2, TINF2, POT1) as well as 18s ribosomal RNA as the housekeeping gene were purchased. The assay was performed in compliance with manufacturer's guidelines and by recommended manufacturer's protocol as can be found on the World Wide Web at superarray.com/Manual/realtimePCR.pdf. The assays were run for N=3 and analyzed using the BioRad GeneX software package. Any values out of range of the threshold cycles or inconsistent within the assay were discarded from analysis.

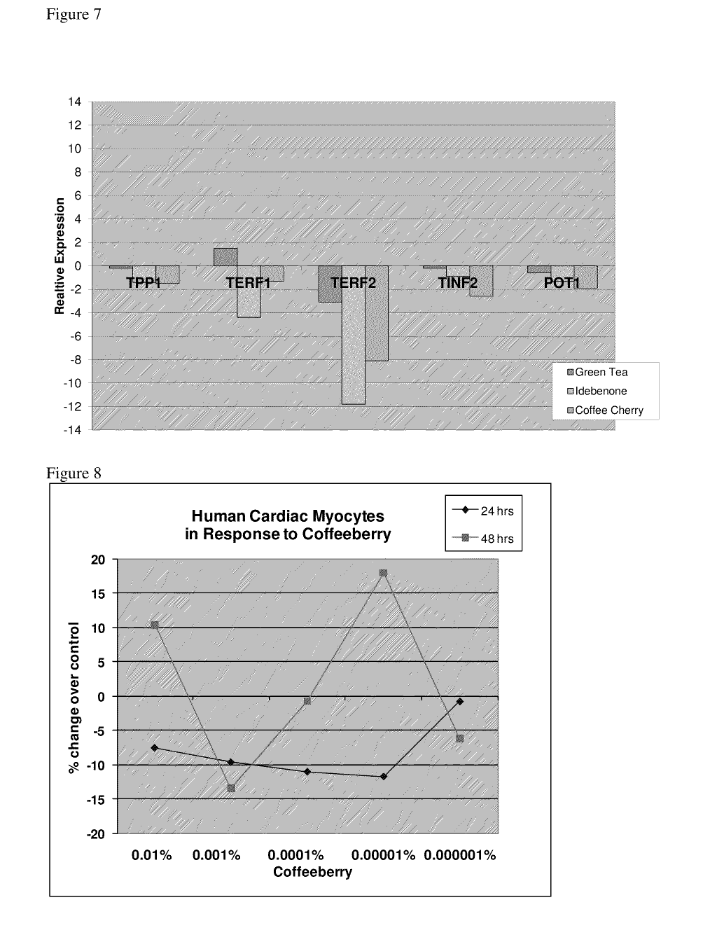

qPCR Primer Assay results: The results from assays were determined using the delta CT method, which uses comparisons of control genes and threshold cycles of genes of interest to generate relative expression values (this process is handled by the GeneX package). Results (average of three experiments) are shown in FIG. 7. The result with the lowest percent error is the TERF2 gene.

As illustrated in FIG. 7, coffee cherry and idebenone affect the expression levels of two genes each (with coffee cherry coming close to effecting a third). Idebenone effected TERF1 and TERF2, while coffee cherry effected TERF2 and TINF2 (a negative regulator of telomere length). This may indicate slightly different mechanisms of action, or different timing/efficiency in the same mechanism, but further study is required.

The largest expression value for all anti-oxidant compounds was seen in the TERF2 assay. TERF2 is said to play a role in the protective activity of telomerase.

Coffee cherry down regulates TINF2, which itself is a negative regulator of telomere length, indicating the potential that coffee cherry in the right concentration can aid in maintaining the length of, or possibly increasing the length of telomeres in cells.

### Example 8

This example illustrates that analysis of the whole human genome of cells exposed to the lifespan modulating agents (such as green tea polyphenols, idebenone and coffee cherry extract, sourced as above) demonstrate longevity/lifespan extension effects in alternate pathways other than telomere length extension.

Cell cultures: A human skin fibroblast cell culture was obtained through the Coriell Cell Repository from the National Institute on Aging Cell Repository. The culture, AG07999, was established from a biopsy of a 32 year old Caucasian female.

Culture media: Cells were grown in Minimal Essential Medium supplemented with 10% fetal bovine serum, 2 mM L-glutamine, 2 mM Glutamax I. During the 24 hour experimental phase, cells were maintained in the same medium, but with only 1% fetal bovine serum. All cultures were incubated at 37° C. with 5% CO₂ in a humidified chamber.

Seeding of cells: On Day 1, 1 ml of 5.0−6.5×10⁵ cells/ml was seeded into each of four 75 cm² flasks containing 20 ml of culture medium.

Experimental phase: On Day 4, the medium was removed by aspiration and replaced with the test condition in 20 ml culture medium but with only 1% fetal bovine serum. Test conditions were 1) 1 μM idebenone and 2) vehicle control. All four conditions had a final vehicle concentration of 0.01% DMSO. On Day 5 after 24 hours of exposure to test conditions, the cells were isolated and the RNA isolated using the RT²q PCR-grade RNA isolation kit (Superarray Bioscience Corporation).

Preparation of RNA: The RNA was isolated using the Qiagen RNEasy Plus Mini Kit according to the manufacturer's protocol which can be found on the World Wide Web at qiagen.com/literature/Default. aspx?Term=&Language=EN&LiteratureType=4&ProductCategory=10162. The RNA was quantified and examined for purity using the 260/280 nm read method in a μQuant spectrophotometer before use in the array.

Analysis of Agilent Whole Human Genome Arrays: RNA taken from both samples was sent to Cogenics, Inc. Morrisville, N.C. Cogenics, Inc. is a leading, state-of-the-art, microarray service provider that facilitates and accelerates transcriptome profiling and gene discovery processes for industrial and academic researchers. Cogenics' procedure for processing samples is as follows: RNA samples are received and analyzed by Cogenics, Inc. using rigorous standardized procedures that are designed to ensure quality and chain of custody. Each sample undergoes a thorough quality analysis using an Agilent Bioanalyzer microfluidics device, and is precisely quantified using a Nanodrop-1000 spectro-photometer. When the Agilent Oligonucleotide Microarray Platform is utilized, samples are fluorescently labeled using the Agilent Low-Input Linear Amplification Kit. Upon completion of this process, the labeled cRNA products are assessed using the same processes described above. These labeled samples are then fragmented and hybridized to oligonucleotide microarrays. The microarrays are washed and then scanned using an Agilent DNA Microarray Scanner with Sure Scan technology. Data is extracted from the images produced by the scanner using Agilent's Feature Extraction software. At this point, the scanned image is visually inspected for defects, and the extracted data is statistically analyzed to ensure quality of the assay. Extracted data and images can be loaded into Rosetta Resolver Gene Expression Data Analysis System for in-depth analysis, both from a quality standpoint, as well as to develop biological understanding.

Agilent Human Genome Array results: The results from the array show the gene expression profile of the entire human genome for a sample treated with 1 μM Idebenone relative to untreated control cells.

Objective 1—Combine the gene expression data from the two fluorophore reversal hybridization replicates to create a single data table representing the biological comparison of interest (Tx compared to UnTx). A table was generated and provided as a tab-delimited text file. This file will contain the log ratio, fold-change, log ratio p-value, etc. for every transcript measured by the microarray.

Using Rosetta Resolver, a single ratio comparison was compiled based on the results of the two fluorophore reversal hybridization performed in the context of this project. The ratios were calculated such that the Tx sample was in the numerator and the UnTx sample was in the denominator. A table was generated, saved as tab-delimited text files, and is provided on the DVD that accompanied this report in the “Objective_{—}1” subdirectory or the “Data_Analysis” directory. These files contain the log ratio, fold-change, log ratio p-value, etc. for every transcript present on the microarray.

Objective 2—Identify differentially expressed transcripts for the comparison generated in Objective 1 using standard criteria (specifically, an absolute fold change value >1.5, a log ratio p-value <0.001). A table for will be generated and provided as a tab-delimited text file. The file will contain the log ratio, fold-change, log ratio p-value, etc. for only the differentially expressed transcripts within the context of the comparison.

The criteria for identification of differentially expressed transcripts were an absolute fold change value >1.5 and a log ratio p-value <0.001. These criteria were applied to the comparison made in Objective 1. A table was generated, saved as tab-delimited text files, and is provided on the DVD that accompanied this report in the “Objective_{—}1” subdirectory or the “Data_Analysis” directory. These files contain the log ratio, fold-change, log ratio p-value, etc. for every transcript that was identified as differentially expressed using the criteria detailed above

The results of this experiment are shown in DATA TABLE 7; this table lists all genes that show a statistically significant change across the human genome. The genes showing statistically significant expression changes can be broken down into subsets, for instance based on the directionality of expression change under treatment, relationships between the genes (e.g., pathway involvement), and so forth.

The following genes show a statistically significant increase in expression after treatment with idebenone and UVB: ARHGAP27, MGC34034, AI446524, LIN28B, PSG9, MPPED2, DCP_{—}22_{—}6, DCP_{—}22_{—}4, DCP_{—}22_{—}0, DCP_{—}22_{—}2, DCP_{—}22_{—}7, LOC440061, THC2319152, HRASLS, BPI, LOC348174, CD1C, ZNF224, TTBK2, C12orf42, ABHD13, AW901755, A_{—}24_P799680, THC2378994, APOBEC3G, CDH7, A_{—}24_P84738, EHF, PARP4, C7orf29, THC2369020, A_{—}24_P289973, THC2378839, MFSD2, FAM40B, A_{—}24_P7820, BC015334, KCNE3, THC2312756, C6orf89, AQP10, AA918648, TUBB4, AK021467, L0051581, MSR1, THC2339455, LOC389025, A_{—}24_P622375, THC2407737, C10orf59, A_{—}24_P942151, A_{—}32_P157622, FAM84B, WWC2, ZNF597, TMEM162, THAP5, CR605947, OTUD6A, THC2440027, WRNIP1, PLA2G3, SOLH, MADCAM1, CPSF6, ENST00000356104, A_{—}23_P10605, THC2378378, A_{—}24_P524164, DCP_{—}22_{—}9, C6orf5, AI652920, KCNMB2, CD34, THC2397757, TXNDC4, THC2448178, CV326037, DAGLA, BANK1, KATNAL2, THC2382717, ITGA2, LOC645561, AA581414, SLC7A11, AL547361, AB011149, THC2343350, PCSK9, KCNH2, C17orf67, CNOT6L, THC2375853, THC2342473, THC2368209, AI709405, THC2322443, AA344632, WNT16, CNPY3, AI161396, DKFZP434P211, BE719776, TIGD3, THC2280741, SCFV, AI873070, THC2408828, THC2235542, SC4MOL, PTPRO, DHCR24, AF086205, STK4, DOCKS, CACNA2D1, THC2316768, SLC16A14, OR10A5, THC2437177, AK094571, A_{—}24_P399341, CELSR1, AK026984, PTPN11, AF086329, THC2400593, IL2, CECR2, KLF8, CRY1, THC2397757, ZNF585A, ENST00000371408, NUDCD1, PGGT1B, DKFZp667E0512, CA13, AK094296, AI051172, ZNF347, THC2324430, GPR132, BCHE, ZNF785, THC2279910, CR598370, LOC344887, ENST00000354343, SEC24A, C8orf61, A_{—}32_P205522, CINP, LRRIQ2, FLJ38973, AF086125, AI192327, ITGB2, BP872463, IL1RAP, TAS2R44, LOC153457, CDCA7, RNF111, A_{—}32_P45087, BC008476, AK023647, AK074181, C14orf49, ZNF516, IL27RA, AA631975, BX448200, THC2419011, IGSF9, PIGL, E2F8, HNRPLL, C10orf27, NACA, TMEM154, TLK1, H43551, ANK1, A_{—}23_P13202, AA725860, ENST00000366971, WO5707, KIAA1432, SCD, KIAA1377, THC2411515, THC2339241, TRIM23, GRIA1, LOC374491, THC2266906, C4orf32, C11orf17, AL571926, A_{—}32_P135790, AI263083, AI925475, THC2360810, NUPL1, FAM60A, AK001164, DCP_{—}1_{—}7, CSTA, PNPLA4, UTS2D, ATP8A2, and C13orf1.

The following genes show a statically significant decrease in expression after treatment with idebenone and UVB: ZNF289, SDHC, HIST1H1A, A_{—}23_P113762, GOLGA2LY1, FLJ43692, EVL, PSAP, KLHDC8B, AKAP12, NFAT5, SPATA13, A_{—}23_P113263, A_{—}32_P220567, SORD, LOC643668, ITSN1, HSP90AB1, LTB4R2, WNT10A, FAM3A, AF212044, DENND4A, MDFI, THC2360912, FOXO4, F1P1L1, THC2290002, HSP90AB3P, STARD3, NOL14, FAM73B, ZC3H13, VCP, DYNC2H1, EHBP1, C6orf204, FABP6, LOC285923, PHKG1, MYO15B, GRLF1, HAB1, ZNF792, PLEKHA2, NLRC5, NIPBL, OTUD7B, NPB, LARS, RASL10B, SAFB2, MALL, GRAMD1B, CDC2L1, UBE2E1, TMEM109, CGNL1, AK000053, DENND1B, ETV4, PKD2, BM455859, BQ772270, HNRNPU, A_{—}24_P84719, RCP9, TNRC15, ACTR2, KIAA0372, DDHD1, FER1L3, RIFT, KLHL17, MCAM, NPFFR1, C1orf144, PPFIBP1, ARFGAP1, WDR31, KLC3, CEP290, TJP2, TCF15, ANGPTL4, LRRC61, CLIP1, SKI, CCDC69, LOC650766, PIFI1, AMAC1L2, RALBP1, NTRK3, ATF71P, KIF14, FLJ22659, ZBED1, TNRC4, EP300, C1orf96, LRIT1, ZDHHC22, A_{—}23_P170719, SMYD4, NOTCH2NL, GNAT1, PIK3R3, LOC729392, AHCTF1, RETN, C7orf51, LOC440836, MIA3, A_{—}23_P44053, TUB, PRPH, TTTY14, FLJ35379, TMC8, DIAPH3, LOC641999, SHC2, THC2278725, KIAA1751, ZNF560, ZNF517, GSC2, and D31825.

The following genes show a statically significant increase in expression after treatment with coffee cherry and UVB: ENST00000302942, ZNF224, DCP_{—}22_{—}0, DCP_{—}22_{—}4, DCP_{—}22_{—}6, DCP_{—}22_{—}7, DCP_{—}22_{—}2, A_{—}24_P799680, THC2319152, LOC348174, DKFZP434P211, AK093508, C12orf42, CDRT15, WNT16, LOC389102, MGC39584, ENST00000356104, MTL5, WNK1, CA503034, LGI2, KRIT1, DCP_{—}22_{—}9, AF334588, H40632, ADAM32, LOC645561, KCNE1, SLC16A12, CDC2L6, DUSP13, PCSK1, BE766438, KIAA1333, TDO2, AF146694, FLJ39653, LOC90586, THC2408033, ZNF516, FLJ40330, THC2437177, T70285, FLJ22662, A_{—}24_P524164, THC2407334, CR605947, THC2375512, SEC24A, THC2406779, AA586832, STYX, BX433326, RAB9B, CA843452, PLEKHK1, LOC728499, BQ000605, ZNF681, AF086125, GDF6, ENST00000306515, MYH8, T62549, AL040873, A_{—}24_P622375, THC2397757, CELSR1, AB011149, DAGLA, THC2440027, FAM133A, AA019203, AW885990, TRIM59, THC2337493, ENST00000379108, AI559980, FLJ21777, LOC646371, GIPR, AA725860, CK818527, AK124806, TBC1D8B, LOC731884, BC015449, N4BP2, A1BG, BC015334, CASC5, EPR1, THC2455353, BU160948, LOC727820, ZNF347, PDE11A, DNAH2, MUPCDH, KIAA0226, AK126245, ZMIZ1, ENST00000380357, BE719776, KRT80, SEPSECS, ROPN1L, AK074181, LOC645238, C8orf61, BCHE, THC2397757, AI263083, LRRC2, POLE, THC2405842, THC2408828, H43551, ATP1A2, TXNDC4, PYROXD1, AF086329, BE644757, THC2266906, ZNF585A, FLJ38973, LOC644053, LOC338328, CR740121, AW167080, BX098411, NACA, CPNE4, AA043564, C22orf24, LOC441208, E2F8, Z28739, LOC128977, THC2343350, PGAP1, ZNF702, BC047110, AL566369, CA866957, AI457687, AW858928, A_{—}24_P144054, A_{—}32_P190944, USF2, C17orf67, FAM133A, BC036599, CYP20A1, GALNT4, AW191706, THC2279825, TMEM154, CA772440, GAS2L3, BC019907, DEPDC1B, LOC401022, ZDBF2, AL571926, ZNF236, PRDX3, MYEOV, AK055302, SHC4, THC2404671, AK092668, AK021606, KIAA1524, ENST00000379131, PRR15, USP6NL, CENPK, MAP4K3, BF195626, TM4SF4, CRY1, DB318210, AI925475, PHTF2, OPCML, RXFP3, AK075186, KIAA1217, HNRPLL, AF086017, and AK131472.

The following genes show a statically significant decrease in expression after treatment with coffee cherry and UVB: A_{—}24_P136155, HSP90AB3P, A_{—}24_P752362, MUM1, HERPUD2, PRO1051, GPRCSC, A_{—}24_P84719, PICK1, SEC14L1, TMEM81, A_{—}24_P229766, FER1L3, RIFT, FLJ10769, MAP4K4, ANAPC7, ARVCF, FLJ23754, TRIB2, TXNRD1, THC2340757, CRY2, PDESA, SIAE, LOC442245, VCP, BM455859, LTB4R, SELENBP1, NOC2L, SKI, GBA2, SAFB2, KLHL17, ZDHHC17, PPFIBP1, RAIL ZC3H13, PSAP, TOM1, PATZ1, ANKRD13D, TSPAN18, THC2360912, RALBP1, A_{—}24_P835943, BU561469, NFATC21P, M74720, CDC2L1, GGT1, WNT10A, CLIP1, HNRNPU, PLEKHA2, C1orf144, THC2356023, TMEM109, KIAA1715, MIA3, COL3A1, HAPLN3, A_{—}32_P149404, PSAP, AKAP12, MAN1C1, MAP1LC3C, EMP1, LGI4, A_{—}24_P922430, C12orf41, THC2361914, HAB1, AJ295984, A_{—}32_P138933, ATP2B3, TAF4B, LOC729392, ANGPTL4, AHCTF1, A_{—}24_P919931, EP300, C10orf39, KLF1, LOC648498, PIK3R3, LOC93349, KLC4, THC2290002, THC2282972, ENST00000342829, ALDH3A1, RASL10B, MGC23270, LYPLA2, TNFRSF21, L0051152, MALL, A_{—}24_P682550, C1QTNF4, ZBED1, NOTCH2NL, SPATA13, A_{—}23_P44053, C14orf115, MKL2, FLJ31401, CCDC69, A_{—}23_P170719, ABCB10, CROCCL2, FMNL1, C3, NFATC2, ELMO2, C9orf139, IGLL1, THC2361491, GIPC2, ZDHHC22, LOC389517, A_{—}24_P3627, ADORA3, LOC644353, LOC440836, DIAPH3, TUB, BX100298, TH, OPRD1, LOC641999, ZNF814, FLJ25328, C11orf21, ENST00000327781, DLG2, PERLD1, THC2408757, THC2317182, KRTAP4-10, BC032901, FLJ35379, CCR2, MAGIX, THC2406786, MTHFD1L, CITED4, and KIAA1751.

The following genes show a statically significant increase in expression after treatment with coffee cherry: GABRA2, THC2319152, A_{—}24_P384379, DCP_{—}22_{—}4, DCP_{—}22_{—}0, DCP_{—}22_{—}7, DCP_{—}22_{—}6, DCP_{—}22_{—}2, A_{—}24_P799680, ZNF224, AK093508, HAL, LOC389102, CREG2, A_{—}23_P134405, C12orf42, AF334588, CDH7, THC2406017, FAM83H, LOC348174, THC2407334, TMEM162, THC2378839, THC2378994, FAM122C, AF034187, CXXC6, OTUD7A, LOC401317, CD28, NUDT10, MFSD2, CASC2, BI836739, THC2342473, MGC39584, LOC400752, TSGA10, AA451708, THC2316936, AA581414, C15orf5, WWC2, KCNE3, A_{—}24_P622375, ZNF587, THAP5, DCP_{—}22_{—}9, AK022479, LOC152217, ZNF585A, KIR3DL1, THC2316768, THC2316649, LGI2, THC2338537, C10orf91, LOC147343, THC2312756, LOC642580, A_{—}32_P71456, CA843452, SUZ12P, LOC727820, UBQLNL, FLJ11996, LOC730057, AK022268, BC008476, HESX1, C20orf74, THC2339455, GDF6, T62549, THC2337372, THC2368209, EPB41L4B, AK021467, CR617865, DKFZP434P211, AA019203, EGR2, THC2437177, C8orf66, SMCHD1, C17orf67, DTWD2, PRR15, THC2281350, L00550643, A_{—}24_P399341, A_{—}24_P289973, NPL, hCG_{—}1776047, THC2339241, NACA, BQ000605, AK023328, THC2397757, LCORL, THC2419011, GATA6, C11orf73, BX412469, COMMD6, GLIPR1L2, CILP, TBC1D8B, OBSCN, A_{—}32_P9931, ZNF516, THC2279910, BE719776, AK074181, DAGLA, LOC645238, THC2381707, ATP1A2, PDE11A, THC2449905, THC2284350, UTS2D, THC2405936, BC047110, PDE11A, PACSIN1, SEMA6D, THC2453866, AK075186, CELSR1, ANK1, AI652920, THC2358845, THC2314215, ZFPM2, THC2378865, DMD, CDCA7, AL833114, LOC645561, ARHGAP20, NANOS1, THC2397757, PRDX3, GIPR, SCRG1, LYSMD4, ENST00000366930, BM986990, GPR132, AK055302, KATNAL2, FLJ11736, MGC24103, AA631975, AI263083, THC2376586, THC2280343, AK092379, KIAA1377, ENST00000256861, LRPSL, DB352368, A_{—}32_P135790, P2RY4, THC2405319, MCTP2, THC2439773, H43551, C8orf61, BC015449, THC2312785, PDE4DIP, HEST, USP6NL, AF086187, PYROXD1, THC2345075, AV702101, DPY19L1P1, IKZF4, BCHE, AF086329, DB318193, CR740121, AK022339, C9orf53, THC2405842, PCDH7, THC2315330, AI192327, THC2381061, TRIM23, THC2409451, USP34, AK092668, AL041007, CB984746, THC2320257, SFRP4, THC2235542, THC2308340, THC2274697, PCDHB16, MAGI2, THC2405710, THC2343350, THC2441367, A_{—}32_P45087, BC036599, LRRC2, TAF4, THC2376418, AK022044, AK026418, OSBPL10, and THC2406944.

The following genes show a statically significant decrease in expression after treatment with coffee cherry: PGS1, A_{—}24_P67268, TBCD, EIF4B, RCN3, SERPINB6, HABP4, A_{—}24_P195454, LOC442013, MAPK13, ADAT1, C21orf2, C4orf23, CR616772, EWSR1, LOC339692, DYRK1A, TEF, ARF3, TPCN2, A_{—}24_P607107, FOXO4, SF3B2, RGS12, DDX54, TP531NP2, STARD3, SNX12, CSRP1, FDPSL2A, AKT1, STATS, TOMM34, PIGG, APOBEC3C, NFATC21P, KIF4A, THC2360912, SAFB2, ANAPC7, TGM2, ATP6V0E2, SMYD4, PMVK, TPCN1, LOC220729, FAM113A, NIPSNAP1, ANP32D, A_{—}24_P375360, MAP7D1, ZNF282, A_{—}23_P205500, HCP5, NBL1, BAX, FLJ23867, EIF4G1, KCTD17, M74720, THC2312955, A_{—}24_P626812, MBD3, CDK5RAP3, COL9A1, CENTG3, KRT16, CAMKK1, FAM63A, TMBIM1, BAT1, NPB, CFL1, CDIPT, ARFRP1, HDAC11, STOML1, STIP1, CNDP2, A_{—}24_P794833, FKBPL, SLC35C2, SMARCD2, BAP1, GRIPAP1, SYNGR2, SEMA4C, CUEDC2, THC2311764, MTCH1, MGC102966, SELENBP1, AP1S1, ATAD3B, NELF, EWSR1, PPIL2, LOC285923, PGS1, LOC442245, MAN1B1, DGKA, LTB4R2, PTPRU, RRBP1, LOC643668, IDH3B, ENO2, CAMK1, ADORA1, PPM1G, DBNL, TJP2, BCAP31, HARS, WDR82, MVK, EWSR1, SDHAL2, GAL3ST4, NFATC3, KRT16, RANGAP1, ZCCHC3, C12orf41, ANXA2P1, GBA2, NDE1, AGPAT6, PIK3IP1, TRAFD1, BAP1, RBM23, FLJ40113, P2RX5, MMP14, PAK1, AURKB, LOC220077, ACADS, LOC441455, PAFAH2, EWSR1, FUS, C1orf216, APOL2, BE379389, SERPINB6, PIAS3, UNC84B, ACP2, C20orf3, YKT6, WDR31, PVR, BRD4, NOC2L, ORC1L, COIL, A_{—}24_P41483, PSMC3, DHTKD1, PPDX, HADHA, ST6GALNAC6, RAPGEF1, ZNF768, PSMD2, FKBP4, PQLC2, U87972, IFIT3, SELO, RFC3, KLHL17, PACSIN3, MAGI1, MAPK12, SMARCA4, OPRL1, TCF25, HSPA6, A_{—}24_P195621, KIAA0913, KLHDC8B, GBA2, C1orf144, NFKBIB, ACAD10, TNIP2, PHKG1, SNX17, SNRP70, CCDC69, A_{—}23_P44053, ETV4, CCNF, LGALS9, SEC24C, FLOT1, VCP, ST6GALNAC2, F2RL3, MALL, PAF1, PSAP, PSAP, TMEM109, SUV39H1, ENO₁B, DNAJB2, TOM1, GRM4, ARFGAP1, VPRBP, POLE, C10orf10, AGPAT6, A_{—}24_P315674, C20orf165, KLHDC4, A_{—}24_P229766, TSR2, SFXN5, KIAA1715, DYSF, NBPF10, ZNF289, A_{—}24_P928031, AA085955, FLJ35379, TUB, MAP2K7, ACE, TTTY14, CCDC19, IGHG1, E4F1, PPME1, KIAA1751, A_{—}24_P913855, A_{—}24_P247303, SMC4, MSH4, GPR120, NKPD1, GRM5, DCLK3, D90075, ADRA2B, THC2374442, A_{—}32_P111919, ANKRD44, and MYCL1.

The data from this experiment is further broken down into additional subsets of genes, provided herewith in DATA TABLE 8 (Telomere complex genes); DATA TABLE 9 (DNA Damage and Repair genes); DATA TABLE 10 (Custom Array I Genes); DATA TABLE 11 (Custom Array II Genes); DATA TABLE 12 (Anti Aging Genes); DATA TABLE 13 (Inflammation Genes); DATA TABLE 14 (Mitochondrial/Cellular respiration/Mitochondrial biogenesis genes); and DATA TABLE 15 (Nitric Oxide Synthase genes).

The genes showing a statistically significant change in expression treatment with coffee cherry extract can also be grouped by art recognized pathways, using for instance Rosetta Resolver Gene Expression Data Analysis System. Genes can also be divided by Rosetta Resolver into “pathways” as they are defined by the Gene Ontology (see, for instance, AmiGO, available on-line at amigo.genontology.org, and particularly amigo.geneontology.org/cgi-bin/amigo/go.cgi). Definitions of the pathways that correspond to the following “pathway” designations also can be found on-line, for instance at the Gene Ontology website.

For the sample analyzed 8 hours after treatment with coffee cherry, statistically significant expression changes were seen genes in the following Primary G0 (Gene Ontology) Term Name “pathways”: ATP catabolic process (2 genes); DNA metabolic process (5 genes); DNA repair (29 genes); DNA replication (33 genes); DNA replication checkpoint (4 genes); DNA replication initiation (11 genes); G1 phase of mitotic cell cycle (6 genes); JAK-STAT cascade (7 genes); RNA elongation from RNA polymerase II promoter (4 genes); UDP-N-acetylglucosamine biosynthetic process (1 genes); actin cytoskeleton organization (24 genes); actin filament bundle formation (3 genes); activation of MAPKKK activity (4 genes); activation of NF-kappaB-inducing kinase activity (5 genes); androgen receptor signaling pathway (11 genes); angiogenesis (24 genes); anion transport (5 genes); anti-apoptosis (23 genes); antigen processing and presentation of endogenous antigen (6 genes); antigen processing and presentation of endogenous peptide antigen via MHC class I (7 genes); apoptosis (49 genes); cellular aromatic compound metabolic process (9 genes); bile acid and bile salt transport (2 genes); blood coagulation (18 genes); cAMP metabolic process (4 genes); calcium-mediated signaling (7 genes); canalicular bile acid transport (2 genes); carbohydrate transport (6 genes); cell cycle (67 genes); cell cycle arrest (32 genes); cell cycle checkpoint (6 genes); cell differentiation (54 genes); cell division (38 genes); cell motion (29 genes); cellular component organization (6 genes); cell proliferation (57 genes); cell-cell signaling (50 genes); cellular response to starvation (2 genes); citrate metabolic process (2 genes); coenzyme A metabolic process (2 genes); cyclooxygenase pathway (2 genes); cytokine-mediated signaling pathway (5 genes); cytoskeletal anchoring at plasma membrane (6 genes); determination of left/right symmetry (2 genes); multicellular organismal development (72 genes); double-strand break repair (5 genes); epidermis development (13 genes); erythrocyte differentiation (3 genes); folic acid transport (4 genes); glucose transport (6 genes); glutamine metabolic process (4 genes); glycerol-3-phosphate metabolic process (4 genes); glycosaminoglycan biosynthetic process (9 genes); glyoxylate cycle (2 genes); growth (6 genes); hemoglobin biosynthetic process (3 genes); heparan sulfate proteoglycan biosynthetic process (5 genes); hindbrain development (3 genes); histone acetylation (4 genes); cellular iron ion homeostasis (7 genes); isocitrate metabolic process (2 genes); lactation (5 genes); megakaryocyte differentiation (2 genes); mevalonate transport (3 genes); mitosis (24 genes); mitotic chromosome movement towards spindle pole (2 genes); monocarboxylic acid transport (3 genes); muscle organ development (23 genes); negative regulation of B cell differentiation (3 genes); negative regulation of DNA replication (2 genes); negative regulation of JAK-STAT cascade (2 genes); negative regulation of Ras protein signal transduction (4 genes); negative regulation of cell differentiation (1 genes); negative regulation of cell proliferation (37 genes); negative regulation of follicle-stimulating hormone secretion (5 genes); negative regulation of interferon-gamma biosynthetic process (3 genes); negative regulation of lipoprotein lipase activity (2 genes); negative regulation of macrophage differentiation (3 genes); negative regulation of phosphorylation (3 genes); negative regulation of programmed cell death (1 genes); negative regulation of transcription (16 genes); negative regulation of transcription from RNA polymerase II promoter (19 genes); nervous system development (47 genes); neural tube closure (2 genes); neutrophil activation (2 genes); nitric oxide mediated signal transduction (3 genes); nucleosome assembly (14 genes); nucleotide catabolic process (2 genes); ovarian follicle development (3 genes); parturition (4 genes); peptide cross-linking (3 genes); activation of phospholipase C activity (6 genes); positive regulation of 1-kappaB kinase/NF-kappaB cascade (17 genes); positive regulation of angiogenesis (5 genes); positive regulation of cell adhesion (4 genes); positive regulation of cell growth (2 genes); positive regulation of cell proliferation (23 genes); positive regulation of fibroblast proliferation (2 genes); positive regulation of follicle-stimulating hormone secretion (3 genes); positive regulation of lipid metabolic process (2 genes); positive regulation of neurogenesis (1 genes); positive regulation of mitotic cell cycle (1 genes); positive regulation of protein kinase activity (3 genes); protein amino acid dephosphorylation (35 genes); protein complex assembly (27 genes); purine base biosynthetic process (4 genes); purine nucleotide biosynthetic process (4 genes); pyrimidine nucleotide metabolic process (3 genes); rRNA processing (13 genes); receptor-mediated endocytosis (13 genes); regulation of MAP kinase activity (2 genes); regulation of bone mineralization (3 genes); regulation of cholesterol biosynthetic process (2 genes); regulation of cyclin-dependent protein kinase activity (19 genes); regulation of inflammatory response (2 genes); regulation of lipid metabolic process (2 genes); regulation of mitosis (5 genes); regulation of ossification (1 genes); regulation of retroviral genome replication (2 genes); regulation of transcription, DNA-dependent (291 genes); regulation of transcriptional preinitiation complex assembly (3 genes); regulation of transforming growth factor beta receptor signaling pathway (3 genes); response to drug (5 genes); response to external stimulus (3 genes); response to stress (16 genes); skeletal system development (22 genes); sphingoid catabolic process (1 genes); sphingosine metabolic process (1 genes); tRNA processing (8 genes); thiamin transport (2 genes); tissue development (2 genes); transcription (226 genes); transcription from RNA polymerase II promoter (36 genes); traversing start control point of mitotic cell cycle (4 genes); and valine metabolic process (3 genes).

For the sample analyzed 24 hours after treatment with coffee cherry, statistically significant expression changes were seen in genes in the following Primary G0 Term Name pathways: DNA damage checkpoint (8 genes); DNA damage response, signal transduction by p53 class mediator resulting in cell cycle arrest (3 genes); DNA metabolic process (11 genes); DNA recombination (18 genes); DNA repair (61 genes); genes DNA replication (66 genes); DNA replication checkpoint (6 genes); DNA replication initiation (12 genes); DNA replication-dependent nucleosome assembly (3 genes); DNA strand elongation during DNA replication (4 genes); G0 to G1 transition (3 genes); G2/M transition of mitotic cell cycle (9 genes); L-glutamate transport (5 genes); MAPK export from nucleus (3 genes); MAPK phosphatase export from nucleus, leptomycin B sensitive (3 genes); NAD biosynthetic process (5 genes); Rho protein signal transduction (10 genes); UDP-N-acetylglucosamine transport (2 genes); UV protection (2 genes); actin modification (3 genes); activation of MAPKK activity (6 genes); activation of MAPKKK activity (4 genes); activation of NF-kappaB-inducing kinase activity (6 genes); age-dependent response to reactive oxygen species (3 genes); cellular aldehyde metabolic process (5 genes); angiogenesis (18 genes); apoptosis (99 genes); arginine catabolic process (6 genes); blood coagulation (29 genes); cAMP metabolic process (4 genes); calcium-mediated signaling (8 genes); canalicular bile acid transport (3 genes); cardiac cell differentiation (2 genes); cell cycle (133 genes); cell cycle arrest (30 genes); cell cycle checkpoint (9 genes); cell death (12 genes); cell differentiation (81 genes); cell division (68 genes); cell growth (15 genes); cell migration (11 genes); cell proliferation (105 genes); cell recognition (6 genes); cell-cell signaling (72 genes); cell-matrix adhesion (28 genes); cell-substrate junction assembly (2 genes); chemotaxis (28 genes); collagen fibril organization (4 genes); complement activation (4 genes); cortical actin cytoskeleton organization (6 genes); cyclin catabolic process (2 genes); cytokine-mediated signaling pathway (8 genes); cytokinesis (10 genes); dTDP biosynthetic process (3 genes); dTTP biosynthetic process (3 genes); deoxyribonucleoside diphosphate metabolic process (2 genes); in utero embryonic development (9 genes); entrainment of circadian clock (3 genes); epithelial to mesenchymal transition (2 genes); erythrocyte differentiation (3 genes); establishment or maintenance of chromatin architecture (13 genes); establishment of mitotic spindle localization (2 genes); ether lipid biosynthetic process (2 genes); germ cell migration (7 genes); glial cell migration (2 genes); glycerol-3-phosphate metabolic process (4 genes); glycoprotein biosynthetic process (3 genes); glycosaminoglycan biosynthetic process (8 genes); gonad development (3 genes); growth (7 genes); growth hormone secretion (3 genes); hemoglobin biosynthetic process (3 genes); histone acetylation (4 genes); hydrogen peroxide catabolic process (3 genes); induction of apoptosis by intracellular signals (9 genes); induction of apoptosis via death domain receptors (6 genes); induction of negative chemotaxis (2 genes); induction of positive chemotaxis (4 genes); insulin secretion (3 genes); internal protein amino acid acetylation (2 genes); intra-S DNA damage checkpoint (2 genes); lactation (4 genes); lysine catabolic process (2 genes); megakaryocyte differentiation (2 genes); mesoderm migration (2 genes); mevalonate transport (4 genes); mitosis (52 genes); mitotic chromosome condensation (5 genes); mitotic chromosome movement towards spindle pole (2 genes); mitotic metaphase plate congression (2 genes); mitotic recombination (4 genes); mitotic sister chromatid segregation (4 genes); mitotic spindle elongation (2 genes); monocarboxylic acid transport (4 genes); motor axon guidance (2 genes); muscle organ development (43 genes); negative regulation of B cell differentiation (3 genes); negative regulation of DNA replication (3 genes); negative regulation of T-helper 2 cell differentiation (3 genes); negative regulation of follicle-stimulating hormone secretion (7 genes); negative regulation of helicase activity (2 genes); negative regulation of interferon-gamma biosynthetic process (3 genes); negative regulation of lipoprotein lipase activity (2 genes); negative regulation of macrophage differentiation (3 genes); negative regulation of phosphorylation (3 genes); negative regulation of cell cycle (37 genes); negative regulation of protein catabolic process (3 genes); negative regulation of sister chromatid cohesion (2 genes); negative regulation of transcription (25 genes); negative regulation of transcription from RNA polymerase II promoter (30 genes); neuron recognition (5 genes); neurotransmitter uptake (4 genes); nitric oxide biosynthetic process (5 genes); nitric oxide mediated signal transduction (5 genes); nucleoside metabolic process (6 genes); nucleosome assembly (32 genes); nucleotide biosynthetic process (7 genes); nucleotide catabolic process (3 genes); nucleotide-excision repair (7 genes); organ morphogenesis (31 genes); organic anion transport (9 genes); ovarian follicle development (4 genes); parturition (4 genes); pentose-phosphate shunt (5 genes); peptidyl-amino acid modification (3 genes); phosphoinositide-mediated signaling (15 genes); positive regulation of JNK cascade (6 genes); positive regulation of T-helper 1 cell differentiation (3 genes); positive regulation of angiogenesis (5 genes); positive regulation of axonogenesis (4 genes); positive regulation of cell adhesion (3 genes); positive regulation of fibroblast proliferation (2 genes); positive regulation of follicle-stimulating hormone secretion (4 genes); positive regulation of glucose import (3 genes); positive regulation of mitotic metaphase/anaphase transition (3 genes); positive regulation of transforming growth factor beta receptor signaling pathway (6 genes); protein amino acid acetylation (4 genes); protein amino acid dephosphorylation (52 genes); protein folding (64 genes); protein hetero-oligomerization (6 genes); protein import into mitochondrial inner membrane (4 genes); protein import into mitochondrial matrix (3 genes); protein localization (7 genes); protein tetramerization (4 genes); proteolysis (109 genes); purine ribonucleoside monophosphate biosynthetic process (4 genes); pyrimidine nucleotide metabolic process (5 genes); pyruvate transport (3 genes); rRNA processing (19 genes); regulation of 1-kappaB kinase/NF-kappaB cascade (3 genes); regulation of MAP kinase activity (3 genes); regulation of Wnt receptor signaling pathway (7 genes); regulation of cyclin-dependent protein kinase activity (18 genes); regulation of mitochondrial membrane permeability (2 genes); regulation of neuron differentiation (5 genes); regulation of protein stability (5 genes); regulation of proteolysis (6 genes); regulation of retroviral genome replication (2 genes); regulation of transcription from RNA polymerase II promoter (71 genes); regulation of transcription, DNA-dependent (447 genes); regulation of transforming growth factor beta receptor signaling pathway (4 genes); response to drug (7 genes); response to external stimulus (4 genes); response to nutrient (10 genes); response to radiation (7 genes); response to superoxide (3 genes); response to unfolded protein (16 genes); sensory organ development (2 genes); skeletal system development (35 genes); spindle organization (8 genes); superoxide metabolic process (5 genes); synapse organization (2 genes); tRNA processing (12 genes); transcription (355 genes); transforming growth factor beta receptor signaling pathway (13 genes); transport (126 genes); traversing start control point of mitotic cell cycle (10 genes); ureteric bud development (3 genes); vacuolar transport (2 genes); valine metabolic process (6 genes); and virus-host interaction (6 genes).

### Example 9

This example describes a whole animal analysis of survival/longevity in fruit flies, after treatment with various antioxidants.

Drosophila cultures: A wingless variant of Drosophila Melanogaster was purchased from Carolina Biological Supply. These flies were fed nutrient media (Formula 4-24) and experimental flies were collected within 18 hours after hatching to ensure the females were virgin.

Culture media: Flies were given a combination of the following:

1) Formula 4-24 without blue coloring for breeding and post exposure to antioxidants.

2) Formula 4-24 with the addition of 1% idebenone or coffee cherry extract for 12 days following hatching. The preparation of the 4-24 media was either w/w ratio with all the dry components (for example 1% idebenone and 99% 4-24) hydrated with sterile water, or the full amount of 4-24 medium was hydrated with an appropriately diluted (coffeeberry was diluted in sterile H₂O, and idebenone was diluted initially in sterile alcohol, and then serially diluted using H₂O) testing compound.

3) Formula 4-24 with the addition of 3% H₂O₂ to oxidatively stress the flies (and decrease the lifespan) post AOX incubation.

4) 20% sucrose solution on filter paper following AOX incubation to serve as a low nutrient media and known lifespan shortening agent.

The flies are hatched in normal media and sexed and transferred to vials containing varying levels (1%, 0.1% or 0.01% of either Idebenone or Coffeecherry extract) or normal media to serve as a control group. The flies remain in the antioxidant media for 12 days. The flies are then transferred to a stressor media (either 3% H2O₂ or 20% sucrose solution) which has been shown to shorten lifespan. All the flies are examined daily and media changed as required until all the flies have died. The date (post 12 day antioxidant incubation) is recorded.

Lifespan results: The cumulative average lifespan of all flies for each group are then computed and compared against the cumulative average of the control (untreated) flies to determine if the Antioxidants increase lifespan. The average lifespan increase is separated by sex as well as combined for both sexes. This lifespan extension is expressed as a percent increase or decrease over control values.

Drosophila cultures: A wingless variant of Drosophila Melanogaster was purchased from Carolina Biological Supply. These flies were fed nutrient media (Formula 4-24) and experimental flies were collected within 18 hours after hatching to ensure the females were virgin.

Culture media: Flies were given a combination of the following: 1) Formula 4-24 without blue coloring for breeding and post exposure to antioxidants, 2) Formula 4-24 with the addition of 1% idebenone or Coffee Cherry extract for 12 days following hatching, 3) Formula 4-24 with the addition of 3% H2O2 to oxidatively stress the flies (and decrease the lifespan) post AOX incubation, and 4) 20% sucrose solution on filter paper following AOX incubation to serve as a low nutrient media and known lifespan shortening agent.

Experimental phase: The experiments performed on the flies generally followed the following pattern: 1) The flies are hatched in normal media and sexed and transferred to vials containing varying levels (1%, 0.1% or 0.01% of either Idebenone or Coffee Cherry extract) or normal media to serve as a control group. The flies remain in the antioxidant media for 12 days. 2) The flies are then transferred to a stressor media (either 3% H2O2 or 20% sucrose solution) which has been shown to shorten lifespan. 3) All the flies are examined daily and media changed as required until all the flies have died. 4) The date (post 12 day antioxidant incubation) is recorded.

Lifespan results: The cumulative average lifespan of all flies for each group are then computed and compared against the cumulative average of the control (untreated) flies to determine if the antioxidants increase lifespan. The average lifespan increase is separated by sex as well as combined for both sexes. This lifespan extension is expressed as a percent increase or decrease over control values and is displayed below.

| PARAMETER | MALE | FEMALE | Average of Both | |
|---|---|---|---|---|
| H₂O₂ | 5.67 days | 5.11 days | 5.32 | days |
| 1% Idebenone + H₂O₂ | 5.32 days | 5.50 days | 5.75 | days |
| 1% CoffeeCherry + | 6.53 days | 7.71 days | 7.10 | days |
| H₂O₂ | | | | |
| % Change Idebenone | +8% | −7% | +8% | |
| % Change Coffeecherry | +15% | +51% | +33% | |
| 20% Sucrose | 9.73 days | 12.2 days | 11.32 | days |
| 1% Idebenone + Sucrose | 12.11 days | 11.7 days | 11.89 | days |
| 1% CoffeeCherry + | 9.25 days | 13.1 days | 11.09 | days |
| Sucrose | | | | |

| | | | |
|---|---|---|---|
| % Change Idebenone | +24% | −4% | +5% |
| % Change CoffeeCherry | −5% | +7% | −2% |

The above table shows the change in lifespan for either coffee cherry or idebenone for Drosophila placed on known longevity decreasing media following 12 days incubation with the antioxidant. Statistical significance was reached on the coffee cherry pretreatment before H₂O₂.

| PARAMETER | MALE | | FEMALE | | Average of Both | |
|---|---|---|---|---|---|---|
| 0.01% Idebenone | Not Completed | | 23.4 | days | 23.4 | days |
| 0.01% CoffeeCherry | Not Completed | | 11 | days | 11 | days |
| 0.1% Idebenone | Not Completed | | 10.2 | Days | 10.2 | days |
| 0.1% CoffeeCherry | 4.1 | days | Not Completed | | 4.1 | days |
| 1% Idebenone | 8.1 | days | 12.4 | days | 10 | days |
| 1% CoffeeCherry | 10.25 | days | 14.7 | days | 12.6 | days |
| Untreated Control | 26.5 | days | 38.4 | days | 33.4 | days |

The above table shows change in average lifespan when Drosophila flies are first hatched and placed directly onto media containing the antioxidant alone.

### Example 10

This example provides a system to capture a representation of the gene expression profiles of cultured human fibroblasts following antioxidant supplementation and exposure to oxidative stress (in the form of UV radiation). This provides a sampling of genes that are significantly altered when given antioxidants and treated with UV when compared to cells only damaged with UV radiation. These genes are indicative of the pathways of repair or protection that are involved with antioxidant supplementation and UV damage.

Cell cultures: A human skin fibroblast cell culture (or cultures) will be obtained through the Coriell Cell Repository from the National Institute on Aging Cell Repository. The initial culture, AG07999, was established from a biopsy of a 32 year old Caucasian female.

Culture media: Cells will be grown in Minimal Essential Medium supplemented with 10% fetal bovine serum, 2 mM L-glutamine, 2 mM Glutamax I. During the 24 hour experimental phase, cells will be maintained in the same medium, but with only 1% fetal bovine serum. All cultures will be incubated at 37° C. with 5% CO₂ in a humidified chamber.

Experimental phase: On Day 1, cells will be seeded into each of four 75 cm² flasks containing 20 ml of culture medium. On Day 4, the medium will be removed by aspiration and replaced with the test condition (Antioxidant supplementation) in 20 ml culture medium but with only 1% fetal bovine serum. Test conditions will be: 1) 1 μM idebenone; 2) Green Tea; 3) Coffeecherry extract; and 4) untreated control.

These conditions will also be duplicated for the same conditions but after stress with UVA, UVB or a combination of UVA and UVB light from a solar simulator/monochrometer. After the determined time points (e.g., 24 hours, 8 hours, and longer—e.g., 144 hours), the cells will be lysed and the RNA will be extracted. The RNA will then be run on Agilent whole human genome microarrays (Kronick, Expert Rev. Proteomics 1(1):19-28, 2004) and the results compiled and analyzed.

During the analysis two objectives will be examined:

Objective 1—Combine the gene expression data from the two fluorophore reversal hybridization replicates to create a single data table representing the biological comparison of interest (Tx compared to UnTx). A table will be generated and provided as a tab-delimited text file. This file will contain the log ratio, fold-change, log ratio p-value, etc. for every transcript measured by the microarray.

Objective 2—Identify differentially expressed transcripts for the comparison generated in Objective 1 using standard criteria (specifically, an absolute fold change value >1.5, a log ratio p-value <0.001). A table for will be generated and provided as a tab-delimited text file. The file will contain the log ratio, fold-change, log ratio p-value, etc. for only the differentially expressed transcripts within the context of the comparison.

The criteria for identification of differentially expressed transcripts will be an absolute fold change value>1.5 and a log ratio p-value<0.005.

### Example 11

This example provides a system to capture a representation of the gene expression profiles of cultured human fibroblasts following antioxidant supplementation and exposure to a second form of oxidative stress (in the form of hydrogen peroxide). This provides a sampling of genes that are significantly altered when given antioxidants as a method for protection against H₂O₂ induced oxidative stress. These genes are indicative of any protective or harmful effects antioxidants have on cells oxidatively stressed with H₂O₂. It will also demonstrate any differences between the mechanisms of action of H₂O₂ induced oxidative stress when compared to UV induced oxidative stress describing possible targets for restorative agents for both types of stress.

Cell cultures: A human skin fibroblast cell culture (or cultures) will be obtained through the Coriell

Cell Repository from the National Institute on Aging Cell Repository. The initial culture, AG07999, was established from a biopsy of a 32 year old Caucasian female.

Culture media: Cells will be grown in Minimal Essential Medium supplemented with 10% fetal bovine serum, 2 mM L-glutamine, 2 mM Glutamax I. During the 24 hour experimental phase, cells will be maintained in the same medium, but with only 1% fetal bovine serum. All cultures will be incubated at 37° C. with 5% CO₂ in a humidified chamber.

Experimental phase: On Day 1, cells will be seeded into 6 well dishes in equivalent cell numbers. On Day 2, the medium will be removed by aspiration and replaced with 600 μM H2O2 in medium. Test conditions will be: 1) 0.0001% coffee cherry extract and 2) H2O2 alone controls.

These conditions may be expanded at a later date to include new extracts/compounds. After the determined time points (e.g., 24 hours, 8 hours, and longer—e.g., 144 hours), the cells will be lysed and the RNA will be extracted. The RNA will then be run on Affymetrix whole human genome microarrays and the results compiled and analyzed.

Affymetrix Human Genome Array results: Basic analysis of the data will involve determining significant fold changes, p-values and basic statistics. Data will be sorted by significance, statistical accuracy and delta value.

### Example 12

This experiment was designed to determine if the tested antioxidant compounds had any effect on mitochondrial biogenesis in cultured human fibroblasts when given antioxidant compounds or stressed with H₂O₂, or a combination of both. Any increase in staining should correlate to an increase in the numbers of mitochondria present since the cells were all seeded at the same confluency and cell numbers.

Cell cultures: A human skin fibroblast cell culture obtained through the Coriell Cell Repository from the National Institute on Aging Cell Repository was used. The culture, AG07999, was established from a biopsy of a 32 year old Caucasian female.

Culture media: Fibroblast cells were grown in Minimal Essential Medium (MEM) supplemented with 10% fetal bovine serum (FBS), 2 mM L-glutamine, 2 mM Glutamax I. During the experimental phase, cells were maintained in the same medium, but with only 1% FBS. All cultures were incubated at 37° C. with 5% CO₂ in a humidified chamber.

Experimental phase: On Day 1, cells were seeded at near confluency into 24 well cluster dishes in 500 μl appropriate medium. On Day 2, all wells were aspirated. Test wells received 500 μl of an antioxidant supplement (coffee cherry extract or idebenone) in medium. Test control wells received 500 μl of medium only. After 24 hours, 48 hours, or 72 hours, wells were aspirated and 300 μl MitoTracker Green in medium was added to each well and incubated for 1 hour. After 1 hour, wells were aspirated and washed 2× with 300 μl appropriate medium and read using a fluorometer.

The percent change (in RFU's or Relative Fluorescence Units) of test wells over controls indicated an increase or decrease in the numbers of mitochondria present in the cells following the antioxidant supplement treatment.

Coffee cherry extract dilutions tested were: 0.01%, 0.001%, 0.0001%, 0.00001%, and 0.000001%. Idebenone dilutions tested were: 10 uM, 1 uM, 0.1 uM, 0.01 uM, 0.001 uM

Results: Using 600 uM H₂0₂ to induce cellular stress for 30 or 60 minutes and coffeeberry (CB) to induce cellular recovery, the following results were found at 24 and 48 hours:

Results: calculate % over control

| | |
|---|---|
| H₂O₂ TX: | H₂O₂ TX: |

| | | | |
|---|---|---|---|
| 30 min | 60 min | 30 min | 60 min |

| | |
|---|---|
| at 24 hours | at 48 hours |

| | | | | |
|---|---|---|---|---|
| +0.001% CB | −13% | −19% | 0.4% | −0.9% |
| +0.0001% CB | −8% | −13% | 0.2% | 1.6% |

### Example 13

This experiment was designed to determine if the tested antioxidant compounds had any effect on mitochondrial biogenesis in cultured human cardiac myocytes when given antioxidant compounds or stressed with H₂O₂, (or a combination of both). An increase in staining should correlate to an increase in the numbers of mitochondria present since the cells were all seeded at the same confluency and cell numbers.

Cell culture: Human cardiac myocytes were obtained through Promocell (Germany). The myocytes were established from a 52 year old Caucasian female.

Culture media: Cardiac myocytes were grown in myocete cell growth medium with supplements recommended by and purchased from Promocell. This medium was used for all phases of growth and experimentation using the myocytes.

Experimental phase: On Day 1, myocytes were seeded at near confluency into 24 well cluster dishes in 500 μl appropriate medium. On Day 2, all wells were aspirated. Test wells received 500 μl of an antioxidant supplement (coffee cherry extract—COFFEEBERRY®) in medium. Test control wells received 500 μl of medium only. After 24 hours or 48 hours, wells were aspirated and 300 μl MitoTracker Green in medium added to each well and incubated for 1 hour. After 1 hour, wells were aspirated and washed 2× with 300 μl medium and read using a fluorometer.

The percent change (in RFU's or Relative Fluorescence Units) of test wells over controls indicated an increase or decrease in the numbers of mitochondria present in the cells following the antioxidant supplement treatment.

Coffee cherry extract dilutions tested were: 0.01%, 0.001%, 0.0001%, 0.00001% and 0.000001%.

MYOCYTES with COFFEEBERRY ® results
Results: calculate % RFU over control

| | 24 hours | 48 hours |
|---|---|---|
| +0.01% CB | −7.6 | 10.3 |
| +0.001% CB | −9.7 | −13.5 |
| +0.0001% CB | −11.1 | −0.8 |
| +0.00001% CB | −11.8 | 17.9 |
| +0.000001% CB | −0.8 | −6.2 |

| | 24 hr Raw data: | | | 24 hr Mean: | | 48 hr Raw data: | | | 48 hr Mean: |
|---|---|---|---|---|---|---|---|---|---|
| +0.01% CB | 955 | 1022 | 968 | 982 | | 886 | 907 | 1029 | 941 |
| +0.001% CB | 874 | 1024 | 981 | 960 | | 554 | 569 | 1089 | 737 |
| +0.0001% CB | 776 | 1051 | 1009 | 945 | | 964 | 535 | 1039 | 846 |
| +0.00001% CB | 834 | 1045 | 935 | 938 | | 946 | 952 | 1120 | 1006 |
| +0.000001% CB | 957 | 1177 | 1028 | 1054 | | 1091 | 770 | 538 | 800 |
| 987 | 864 | 1184 | 1217 | 1063 | Controls | 939 | 1035 | 586 | 853 |

This data is also shown in FIG. 8.

It appears there is biphasic response of the mitochondria to coffee cherry extract. Without wishing to be limited to any particular mechanism, depending on the dosage coffee cherry can function to either increase or decrease the number of mitochondria.

### Example 14

This example provides a method that can be used to determine the rate of mitochondrial respiration or efficiency following antioxidant supplementation.

Cell culture: Human cardiac myocytes were obtained through Promocell (Germany). The myocytes were established from a 52 year old Caucasian female.

Culture media: Cardiac myocytes will be grown in myocete cell growth medium with supplements recommended by and purchased from Promocell. This medium will be used for all phases of growth and experimentation using the myocytes.

Experimental phase: On Day 1, myocytes will be seeded at near confluency into 24 well cluster dishes in 500 μl appropriate medium. On Day 2, all wells will be aspirated. Test wells will receive 500 μl of an antioxidant supplement (coffee cherry extract) in medium. Test control wells will receive 500 μl of medium only. After the determined time points (using the same time points as previous experimentation 24 hours, 8 hours, and if desired at 144 hours to mimic apple stem cell paper/patent of exposure to test conditions), the cells will have mitochondrial efficiency measured using the Clark electrode or Seahorse XF24 Flux Analyzer according to the recommended protocols

Coffee cherry extract dilutions to be tested: 0.01%, 0.001%, 0.0001%, 0.00001% and 0.000001%.

Mitochondrial Efficiency results: Basic analysis of the data will involve determining significant changes, p-values and basic statistics.

### Example 15

Chlorogenic acid challenge experiment for RNA isolation for microarray analysis

Human dermal fibroblasts (ag07999) were seeded at near confluency in 6-well dishes in 4 ml MEM, 10% FBS/well. 24 hours after seeding, wells were aspirated and 3 ml chlorogenic acid dilution in MEM, 1% FBS added, or MEM, 1% FBS only for control wells. Chlorogenic acid (Acros Organics, Geel, Belgium) dilutions used were: 0.005%, 0.0005%, 0.00005%, 0.000005% and 0.0000005%. 24 hours after chlorogenic acid challenge, wells were aspirated, 1 ml trypsin-EDTA added to each well, swirled and aspirated. Trypsin-EDTA was again added to each well and cells retrieved with MEM 10% FBS when released from the substratum and centrifuged to a pellet.

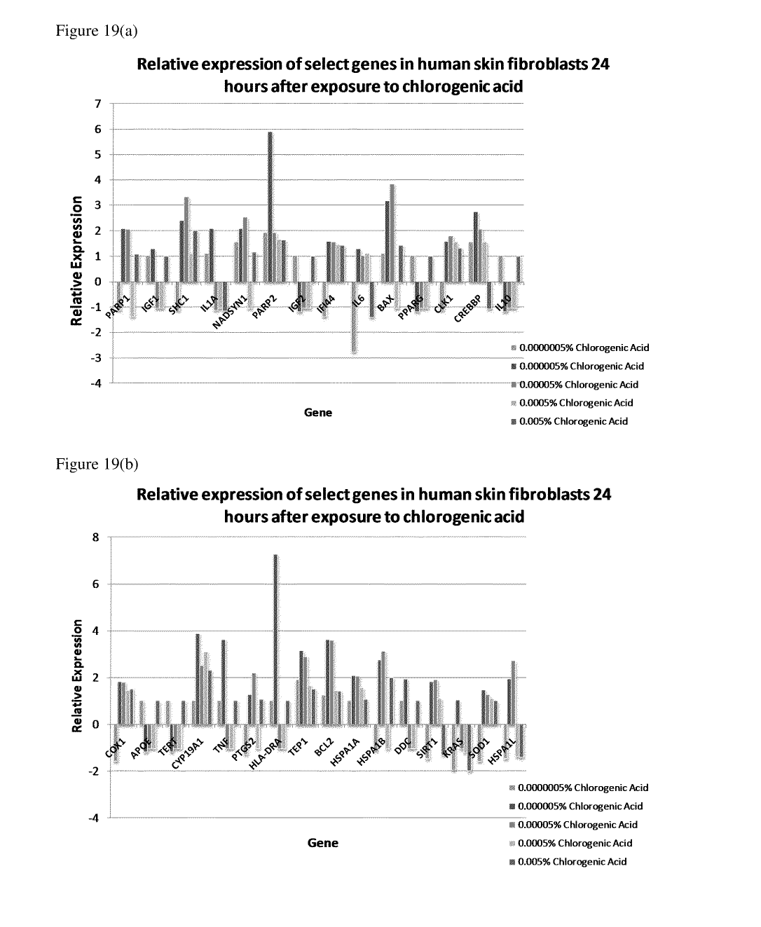

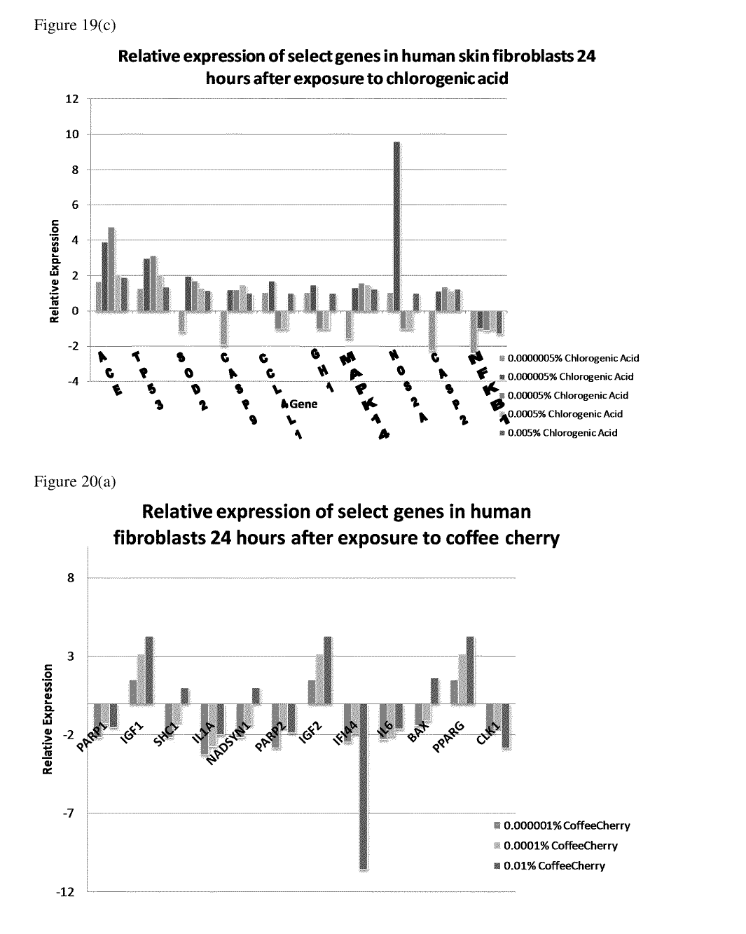

RNA was collected from the harvested cells according to protocol using RT2 qPCR-Grade RNA Isolation Kit from SABiosciences Co. The RNA was then used to create cDNA (using a First Strand Synthesis Kit, SABiosciences, and following the manufacturer's instructions) and run on the BioRad iCycler RT-PCR machine using Custom RT-PCR Microarray (CAPH09464A, SABiosciences) (Array 1) and SYBR Green Reaction Mix (SABiosciences). Data from Example 16 are shown in FIG. 19; additional results are described and analyzed below.

### Example 16

### COFFEEBERRY® Challenge Experiment for RNA Isolation for Microarray Analysis

Human dermal fibroblasts (ag07999) were seeded at near confluency in 6-well dishes in 4 ml MEM, 10% FBS/well. 24 hours after seeding, wells were aspirated and 3 ml coffeeberry dilution in MEM, 1% FBS added, or MEM, 1% FBS only for control wells. COFFEEBERRY® dilutions used were: 0.1%, 0.01%, 0.005%, 0.001%, 0.0005%, 0.0001%, 0.00005%, 0.00001%, and 0.000001%. 24 hours after coffeeberry challenge, wells were aspirated, 1 ml trypsin-EDTA added to each well, swirled and aspirated. Trypsin-EDTA was again added to each well and cells retrieved with MEM 10% FBS when released from the substratum and centrifuged to a pellet.

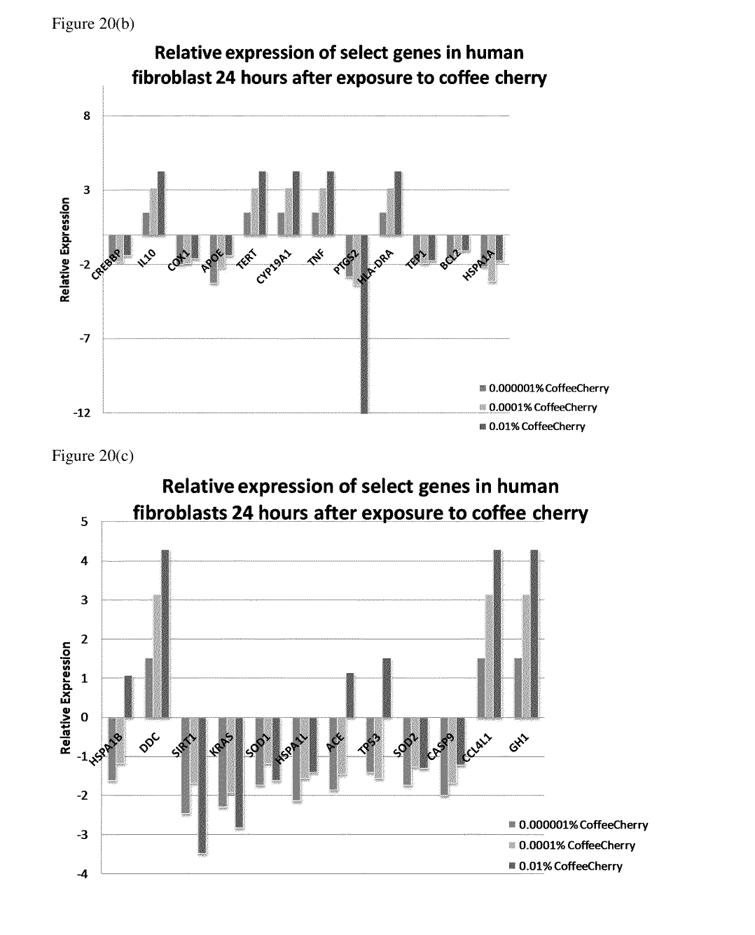

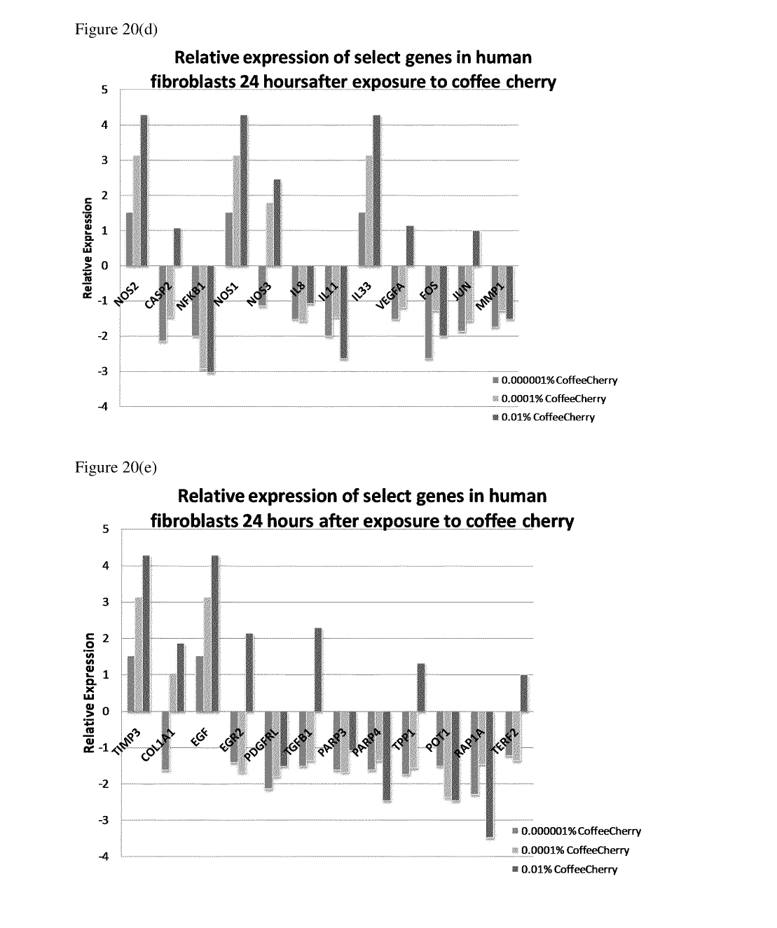

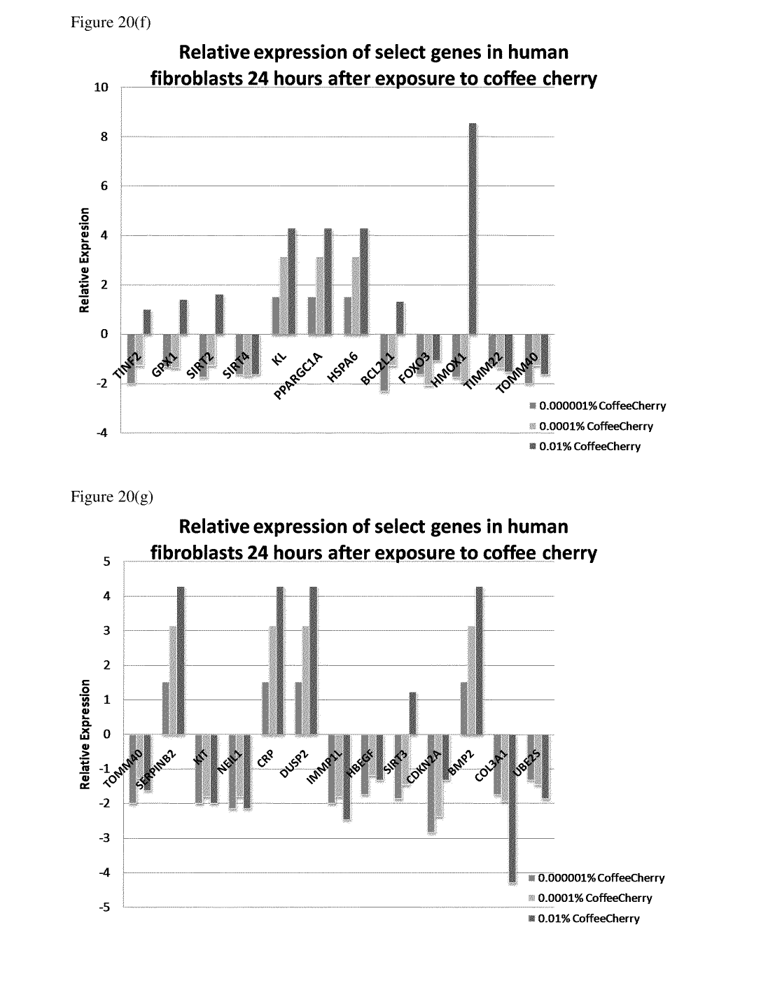

RNA was collected from the harvested cells according to protocol using RT2 qPCR-Grade RNA Isolation Kit from SABiosciences Co. The RNA was then used to create cDNA (using a First Strand Synthesis Kit, SABiosciences) and run on the BioRad iCycler RT-PCR machine using Custom RT-PCR Microarray (CAPH09464A, SABiosciences) (Array 2) and SYBR Green Reaction Mix (SABiosciences). Data from Example 15 are shown in FIG. 20; additional results are described and analyzed below.

### Results and Discussion for Examples 15 and 16

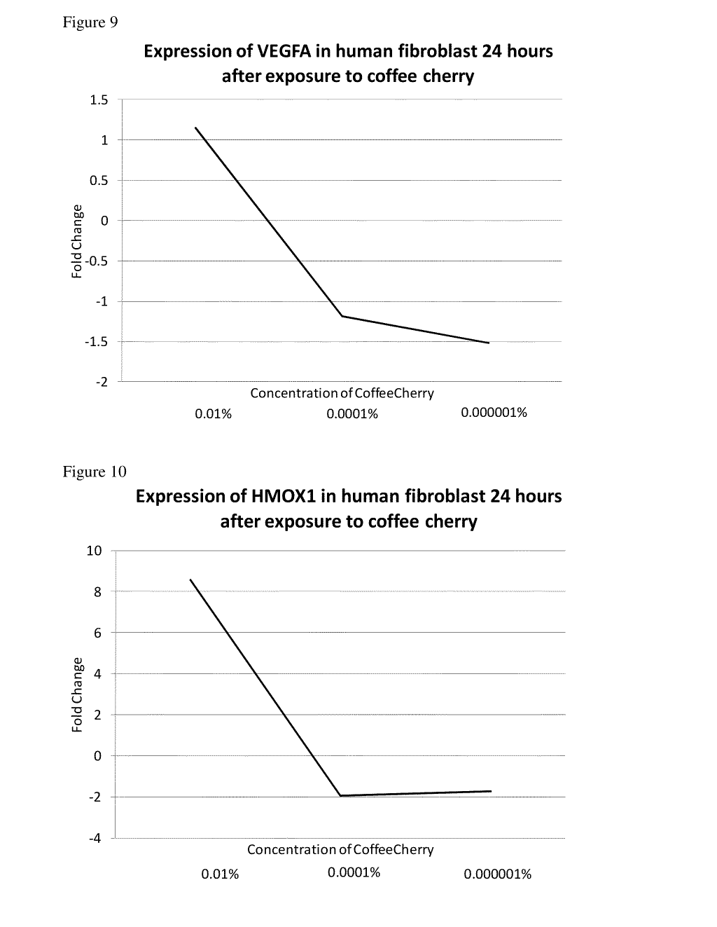

FIGS. 9-18 show changes in the expression of specific genes (VEGFA, HMOX1, CCL4L1, DDC, NOS2A, SIRT1, SIRT2, SIRT3, SIRT4, TERT, PTGS2, and IFI44) in human fibroblasts treated with coffee cherry extract (0.000001%, 0.0001% or 0.01%) or chlorogenic acid (0.000005%, 0.00005%, or 0.005%).

FIG. 9 (VEGFA; VEGF; Vascular Endothelial Growth Factor) shows the change in relative expression of VEGFA with decreasing concentrations of coffee cherry extract. A relatively high concentration of coffee cherry extract (0.01%) induces the expression of this protein, while lower levels (0.0001% and 0.000001%) actually repress expression of this protein. VEGFA is a homodimeric glycoprotein of relative molecular mass 45,000, is the only mitogen that specifically acts on endothelial cells. It may be a major regulator of tumor angiogenesis in vivo. VEGFA is a candidate hormone for facilitating glucose passage across the blood-brain barrier under critical conditions, tumor angiogenesis, VEGF and IL6 are produced together in the intraocular tissues and that both are involved in the pathogenesis of diabetic macular edema. Increasing expression of VEGF can be useful for improving wound healing, while decreasing expression can be important for treating macular degeneration of the retina. It is noted that TGFB behaves similarly with regard to expression responses to antixoidants.

VEGF is involved in stimulating and inhibiting growth of new blood vessels, which makes this a particularly important gene in wound healing and cancer, as well as macular degeneration of the retina and other diseases. Thus, the discovery herein that antioxidant compositions such as coffee cherry and chlorogenic acid can be used to either induce or repress VEGF expression enables methods of treating each of these conditions.

Collagen 1A1 (the dominant from of collagen in skin) exhibits a similar dosage response to VEGF, so it is believed that (relatively) higher concentrations of antioxidants could be used to improve fine lines, wrinkles, and other aspects of skin. It may be particularly beneficial to reduce expression or activity of MMP-1 collagenase concurrently, so it is particularly useful that MMP1 is down regulated (at about the same degree for all concentrations). This is particularly useful for anti aging in skin or repairing aging, and may be useful to reverse, inhibit, delay, or offset defects of the skin's dermal matrix, including for instance fine lines, wrinkles, sagging, tone, and so forth.

FIG. 10 (HMOX1; Heme Oxygenase 1) shows the change in relative expression of HMOX1 with decreasing concentrations of coffee cherry extract. A relatively high concentration of coffee cherry extract (0.01%) induces the expression of this protein, while lower levels (0.0001% and 0.000001%) actually repress expression of this protein—similar to the pattern observed for VEGFA. HMOX1 catalyzes the rate limiting step in the catabolism of heme to form biliverdin, which is subsequently converted to bilirubin by biliverdin reductase, free iron, and carbon monoxide. Heme oxygenase shows antioxidative effects and induced Hmox1 may protect against lipopolysaccharide-induced septic shock.

FIG. 11 (CCL4L1; a.k.a. LAG1) shows the change in relative expression of HMOX1 with increasing concentrations of coffee cherry extract and chlorogenic acid (a component of coffee cherry extract). CCL4L1 is one of several cytokine genes clustered on the q-arm of chromosome 17. Cytokines are a family of secreted proteins involved in immunoregulatory and inflammatory processes. This protein is similar to CCL4 which inhibits HIV entry by binding to the cellular receptor CCR5. The copy number of this gene varies among individuals; most individuals have 1-5 copies in the diploid genome, although rare individuals do not contain this gene. It has been suggested that the most effective anti-HIV drugs would be those that increase expression of whichever CCL4 protein, i.e., ACT2 or LAG1, has the highest affinity for CCR5 identified B-cell lines that express predominantly LAG1. Coffee cherry exposure results in significantly increased expression of CCL4L1 in a dose dependent manner, which can now be exploited for altering immune response and particularly for treatment of HIV infection. In contrast, chlorogenic acid displays a non-linear dosage response, with the middle dosage (0.00005%) yielding a marked repression of CCL4L1.

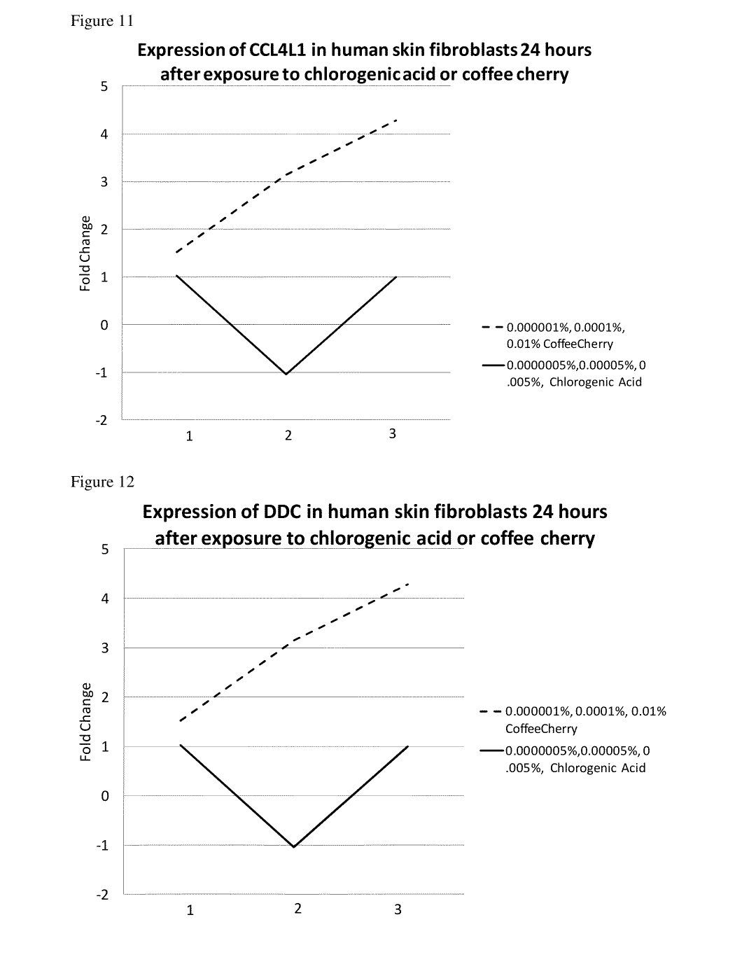

FIG. 12 (DDC; Dopa Decarboxylase) shows the change in relative expression of DDC with increasing concentrations of coffee cherry extract and chlorogenic acid (a component of coffee cherry extract). DDC is an enzyme implicated in two metabolic pathways (biosynthesis for bioamines and catecholamines), synthesizing two important neurotransmitters: dopamine and serotonin. A polymorphism in tyrosine hydroxylase (TH; OMIM 191290), the rate-limiting enzyme in the synthesis of catecholamines, is associated with variation in human longevity. The ability to increase expression with coffee cherry illustrated in this figure provides a method for altering the production of neurotransmitters and may be exploited in the treatment of depression, Parkinson's disease, lifespan extension and a host of other clinical diseases and metabolic functions associated with aging.

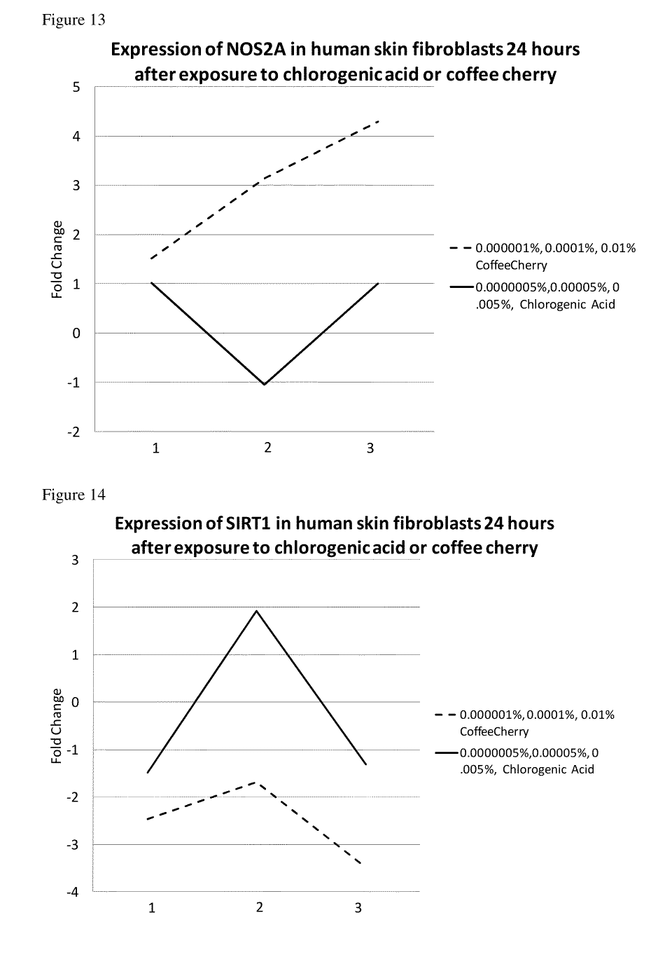

FIG. 13 (NOS2A; Nitric Oxide Synthase 2A) shows the change in relative expression of NOS2A with increasing concentrations of coffee cherry extract and chlorogenic acid (a component of coffee cherry extract). Nitric oxide (NO) is a messenger molecule with diverse and very important functions throughout the body. In the brain and peripheral nervous system, NO displays many properties of a neurotransmitter; it is implicated in neurotoxicity associated with stroke and neurodegenerative diseases, neural regulation of smooth muscle, including peristalsis, and penile erection. NO is also responsible for endothelium-derived relaxing factor (EDRF) activity regulating blood pressure. In macrophages, NO mediates tumoricidal and bactericidal actions, as indicated by the fact that inhibitors of NO synthase (NOS) block these effects. NO plays a significant role in mitochondrial biogenesis as well. The ability to modulate NO expression either up or down (as illustrated in this figure) can have important role in lifespan, mitochondrial biogenesis, healthy longevity and good health in general. Coffee cherry displays a dose dependent increase in expression, whereas chlorogenic acid showed biphasic response with up or down regulation of expression being dependent upon the dose.

FIG. 14 (SIRT1, Sirtuin1) shows the change in relative expression of SIRT1 with increasing concentrations of coffee cherry extract and chlorogenic acid (a component of coffee cherry extract). SIRT1 is a stress-response and chromatin-silencing factor. It is an NAD(+)-dependent histone deacetylase involved in various nuclear events such as transcription, DNA replication, and DNA repair. SIRT1 protein binds and deacetylates the p53 protein the deacetylase activity accounts for silencing, recombination suppression, and extension of life span in vivo. Furthermore, SIRT1 repressed p53-dependent apoptosis in response to DNA damage and oxidative stress. The SIRT1 gene is turned on by a caloric restriction diet, and this protects cells from dying under stress and may extend lifespan. Coffee cherry and chlorogenic acid each demonstrated a non linear dose response curve for increasing or decreasing expression of SIRT1 with chlorogenic acid being entirely decreasing expression but coffee cherry could either increase or decrease (and thus modulate) the expression of SIRT1.

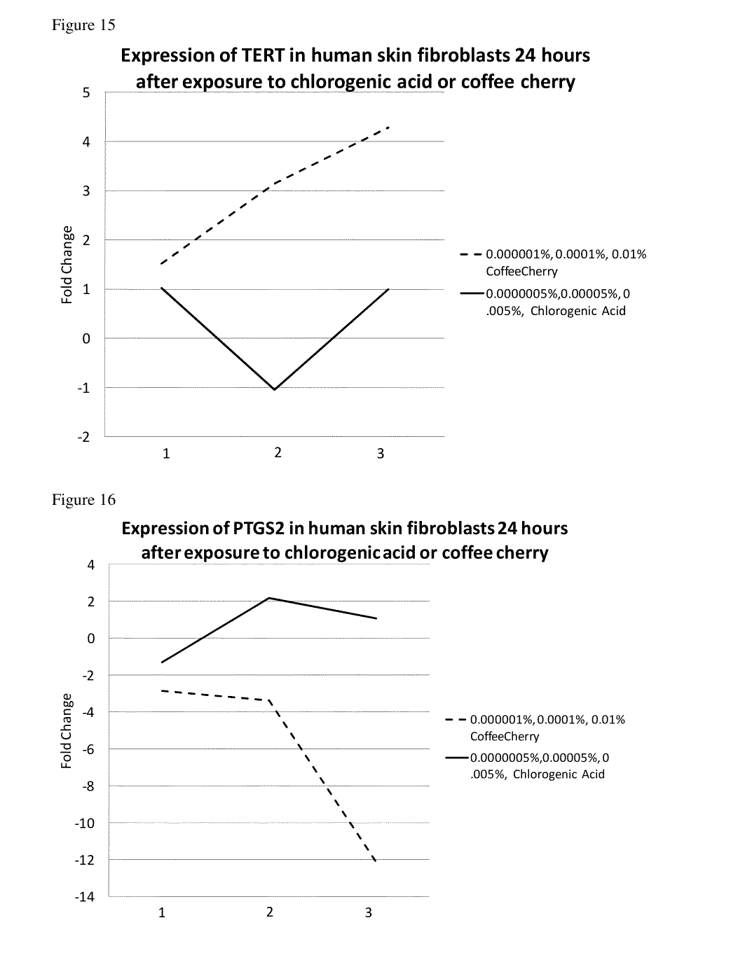

FIG. 15 (TERT; Telomerase Reverse Transcriptase) shows the change in relative expression of TERT with increasing concentrations of coffee cherry extract and chlorogenic acid (a component of coffee cherry extract). Coffee cherry showed a linear dose response whereas chlorogenic acid showed a non linear response; the relative TERT gene expression increased with increasing coffee cherry concentration, but could be either induced or reduced with different amounts of chlorogenic acid. The ability to enhance telomere maintenance with coffee cherry may increase lifespan.

FIG. 16 (PTGS2; Prostaglandin-Endoperoxide Synthase 2) shows the change in relative expression of PTGS2 with increasing concentrations of coffee cherry extract and chlorogenic acid (a component of coffee cherry extract). A major mechanism for the regulation of prostaglandin synthesis occurs at the level of cyclooxygenase, also known as prostaglandin-endoperoxide synthase. PTGS1 is involved in production of prostaglandins for cellular housekeeping functions, whereas PTGS2 is associated with biologic events such as injury, inflammation, and proliferation. PTGS2 encodes the pro-inflammatory cyclooxygenase 2 enzyme believed to be the rate-limiting step in the synthesis of prostaglandins, important mediators of inflammation. Increasing amounts of coffee cherry extract induced an increase in PTGS2 gene expression until a plateau was reached at the doses tested. In contrast, chlorogenic acid showed a more linear type of dose response that produced at higher concentrations a significant decrease in PTGS2 gene expression. Inflammation, especially chronic inflammation, is associated with many diseases and directly or indirectly with reduced lifespan (or particularly reduced healthy lifespan), so the ability to significantly decrease the expression of PTGS2 as illustrated in this figure is an important ‘anti inflammatory’ option. Also it is an option to decrease inflammation without the use of steroids and their attendant adverse side effects, as chlorogenic acid may function partially as a ‘non steroidal anti inflammatory’ compound that is derived from botanical sources.

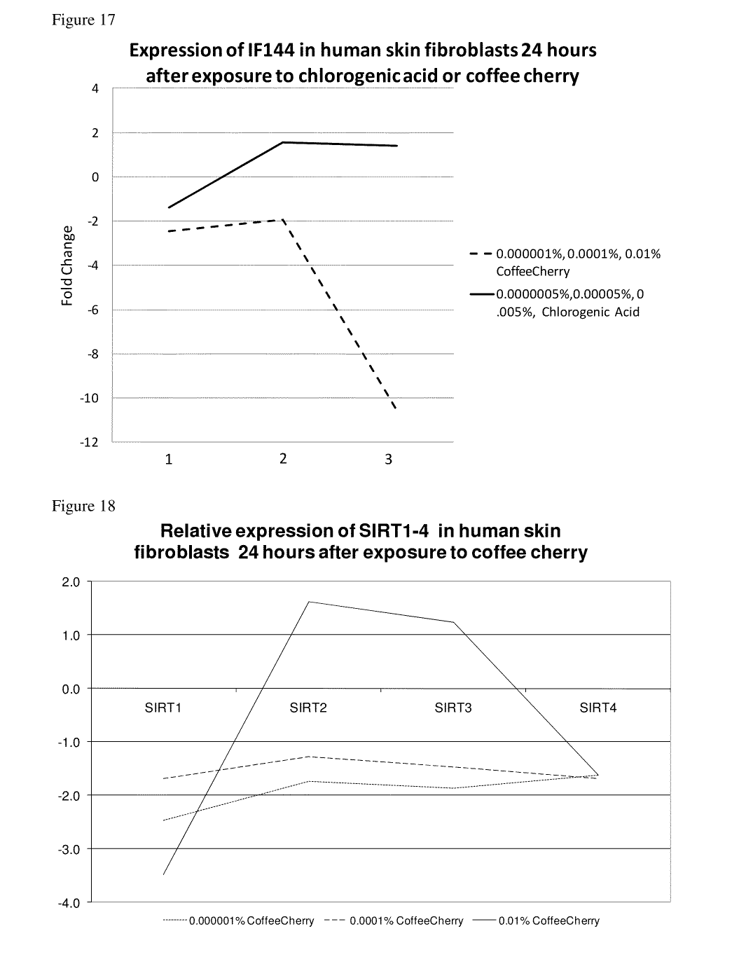

FIG. 17 (IFI44 (a.k.a. p44); Interferon Induced Protein 44) shows the change in relative expression of IFI44 with increasing concentrations of coffee cherry extract and chlorogenic acid (a component of coffee cherry extract). IFI44 is induced in the liver of chimpanzees infected with hepatitis C or hepatitis D virus, but not in the liver of those infected with hepatitis B virus. Others have suggested that IFI44 induction is the result of interferons produced in response to viral infection. IFI44 is inducible by interferon (IFN)-alpha (OMIM: 147660) and IFN-beta (OMIM: 147640), but not by IFN-gamma. Chlorogenic acid may significantly decrease expression and reduce interferon production whereas coffee cherry may increase expression thus allowing the ability to modulate up or down the gene expression of this interferon.

FIG. 18. Relative expression of SIRT1-4 in human skin fibroblasts 24 hours after exposure to coffee cherry extract. The SIRT genes code for proteins which are enzymes which deacetylate proteins that contribute to cellular regulation such as reaction to stressors or regulating longevity. Coffee cherry down regulates SIRT1 and SIRT4 at all the tested concentrations but can either down or up regulate expression of SIRT2 and SIRT3 depending on the concentration.

The genes from Examples 15 and 16 were also broken out into various smaller sets for comparative analysis:

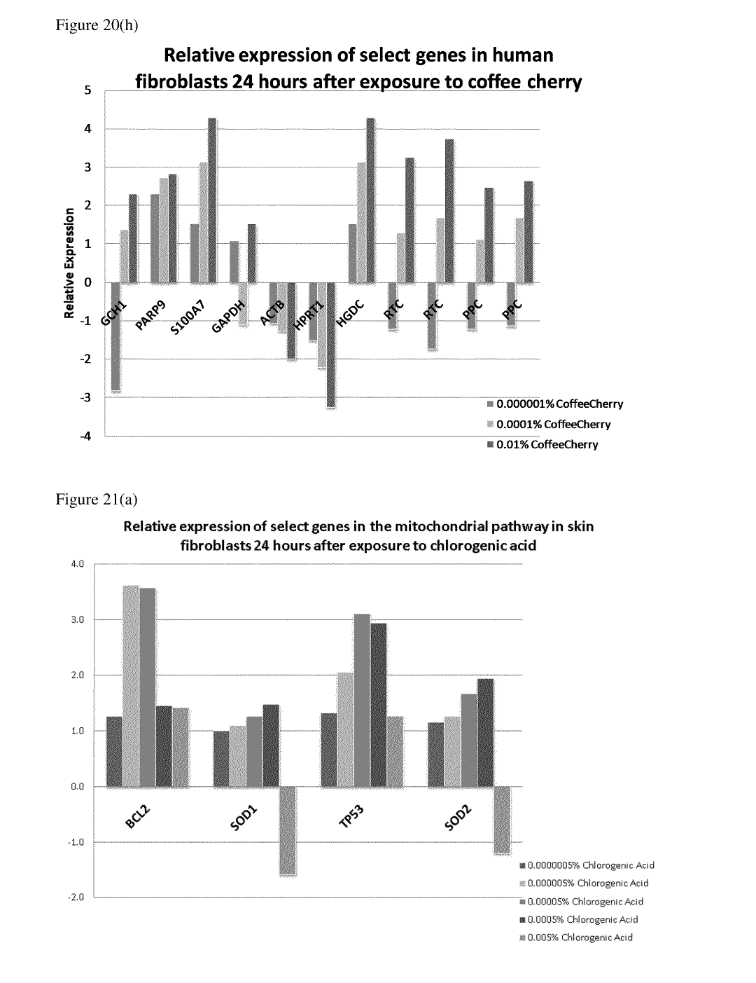

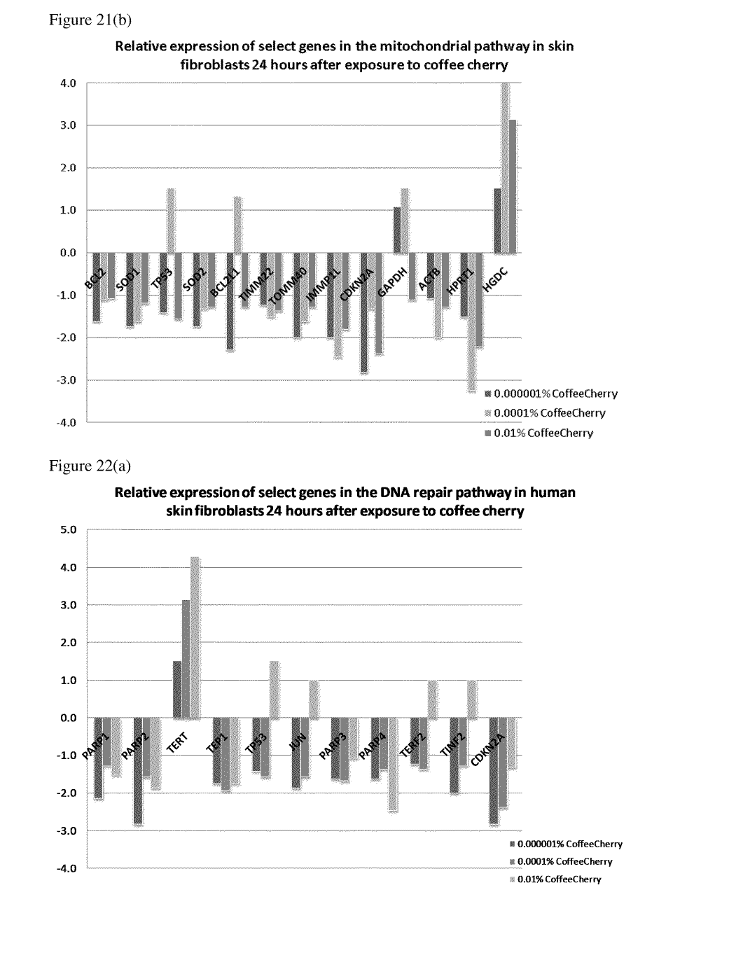

FIG. 21 shows the relative expression of select genes in the mitochondrial function/biogenesis pathway after exposure to chlorogenic acid (FIG. 21a) or coffee cherry extract (FIG. 21b). The response for these genes involved in mitochondrial pathways and biogenesis are essentially opposite for coffee cherry (which primarily increases gene expression) and chlorogenic acid (which primarily decreases gene expression). Not only are the directions of the change in gene expression essentially opposite, but in examining individual gene expression patterns at different doses or concentrations, the chlorogenic acid shows a consistent pattern of a bell shaped dose response curve with the greatest expression at middle range of the tested doses while the coffee cherry show more variation in dose response and is non linear for many but not all the genes illustrated.

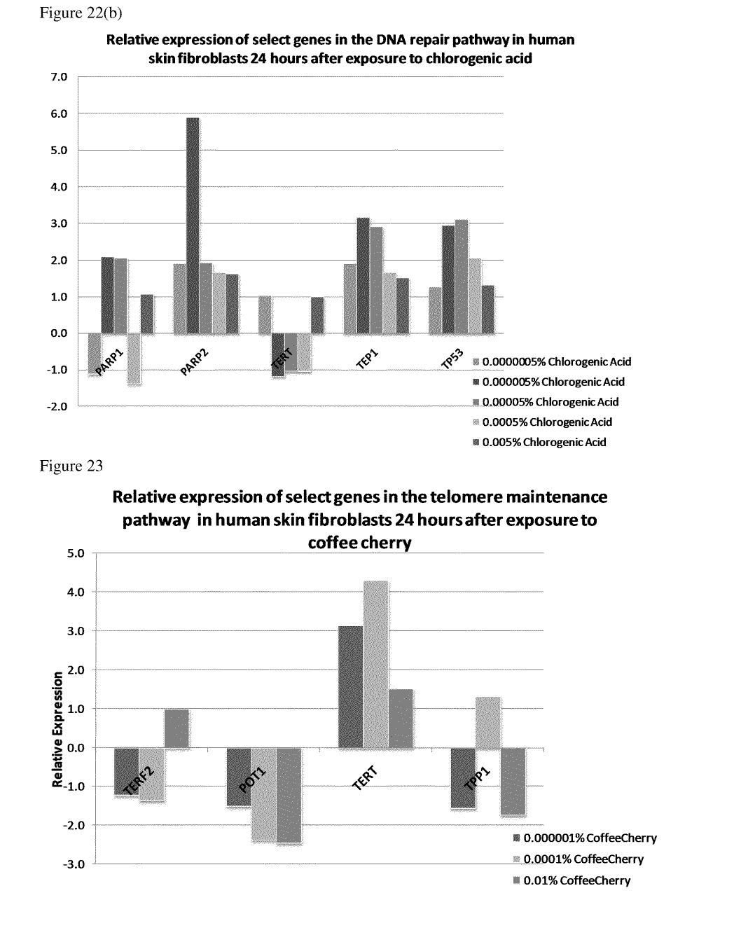

FIG. 22 shows the relative expression of select genes in the DNA repair pathway after exposure to chlorogenic acid (FIG. 22a) or coffee cherry extract (FIG. 22b). With the exception of TERT the coffee cherry overall pattern is essentially one of decreasing gene expression at lower concentrations but the amount of decrease becomes less or may even become positive increase in gene expression with increasing dose. TERT shows a linear increase in gene expression with increasing dose or concentration which is favorable for DNA and telomere function repair. In contrast, chlorogenic acid shows a non linear dose response for all the illustrated genes and in the case of TERT actually decreases gene expression which is opposite of the coffee cherry effect.

FIG. 23 shows the relative expression of select genes in the telomere maintenance pathway after exposure to coffee cherry extract. Coffee cherry produces an increase in TERT gene expression and a decrease in POT1, both of which are associated with enhanced telomere maintenance and possible increased longevity, whereas a decrease in TERF2 is contraindicated for this goal. There is an increase in POT1 downregulation with higher dose or concentration but a non linear response of TERT as concentration increases; however, both remain in a favorable gene expression pattern for increasing longevity. TERF2 however at lower concentrations is unfavorable but changes to favorable for increasing longevity at higher concentrations. TPP1 is variable. The POT1 (Protection Of Telomere) gene forms an important POT1-TPP1 telomere complex which is a telomerase processivity complex. TPP1 expression is dose response variable. At highest concentrations TERF2, POT1, TPP1 and TERT gene expression all favor enhanced telomere maintenance and increased longevity.

It is also noted that KL (Klotho) expression increases with increasing levels of coffee cherry (similar to TERT), and as with TERT, more KL is good for longevity and healthy lifespan.

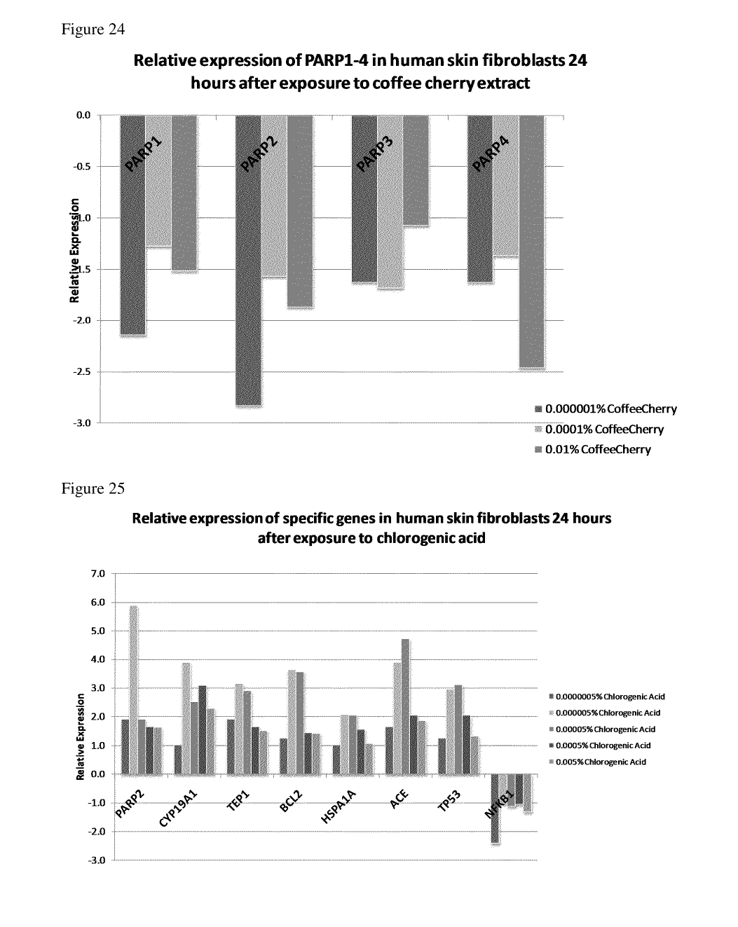

FIG. 24 shows the relative expression of the PARP1-4 genes after exposure to coffee cherry extract. Among other things, PARP activates signalling to release Apoptosis Inducing Factor (AIF) from mitochondria resulting in caspase independent pathways for apoptosis/programmed cell death and may have a role related to DNA repair and PARG gene function. Members of the PARP family typically interact with each other. Decreased expression of PARP genes may be beneficial in extending cell lifespan which is of value for healthy cells, but in contrast for diseased or cancerous cells the ability to increase PARP expression and promote apoptosis for the more rapid death of these unhealthy cells may also be desirable. Thus, modulation either to decrease or increase PARP expression can be useful for overall longevity of a tissue, organ or organism.

FIG. 25 is a graph illustrating the relative expression of specific genes in human skin fibroblasts 24 hours after exposure to chlorogenic acid which demonstrate a classic bell shaped pattern for dose response that indicates a single directional change and then return to baseline after a peak expression level. As the doses increase, the gene response either increases or decreases until a peak expression level is reached. Beyond that dosage any increases in concentration of the compound gives “diminishing returns” or a lessening of the effect. This effect is either an upregulation or a downregulation, not bi directional.

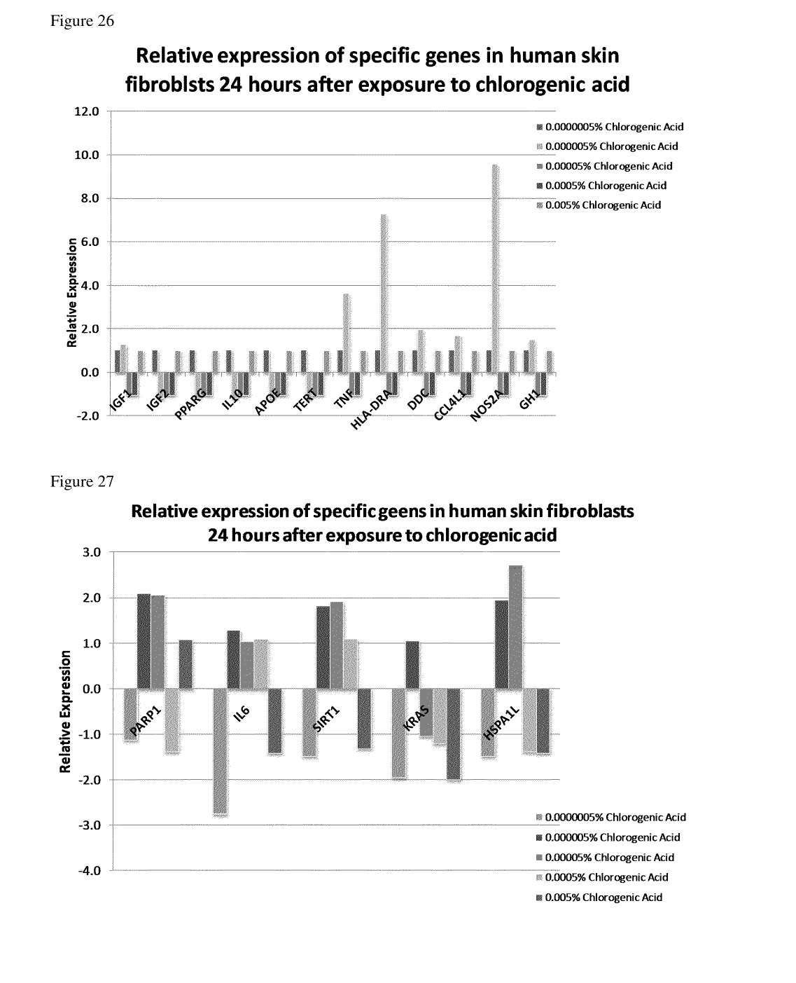

FIG. 26 is a graph illustrating the relative expression of specific genes in human skin fibroblasts 24 hours after exposure to chlorogenic acid which demonstrate a classic bell shaped pattern for dose response that begins as a negative expression value and as the dosage increases it passes through the zero expression value and has an positive expression value until a threshold dose is reached and then returns to the other side of the axis similar to the starting dose. This is the first type of bi-directional dose response noted.

FIG. 27 is a graph illustrating the relative expression of specific genes in human skin fibroblasts 24 hours after exposure to chlorogenic acid which demonstrate a classic bell shaped pattern for dose response that begins as a positive expression value and as the dosage increases it passes through the zero expression value and has an negative expression value until a threshold dose is reached and then returns to the other side of the axis similar to the starting dose. This is the second type of bi-directional dose response noted.

Additional general conclusions and connections can be drawn based on the data provided by Examples 15 and 16.

In generally, coffee cherry seems to have a ‘linear’ dose response curve, whether that is headed towards upregulation or downregulation. A few of the observed linear responses go from down to up, or up to down regulation, such that ‘opposite’ effects at observed different doses of the same coffee cherry. The genes that show linear expression changes with dosage that are always induced (upregulated) or always repressed (downregulated) are basic traditional ‘drug dose response curves’—in general the higher the dose the greater the response (though it is noted that side effects may or may not mirror dose). However, there are also genes that show clearly responses that are not simply linear—and these highlight that it can be very important to carefully regulate the dosage of the lifespan influencing agent.

Overall, chlorogenic acid also has a very consistent pattern—in that there are almost no linear responses to changing concentration. The dosage response curves for chlorogenic acid are essentially all bell shaped curves, either all above the baseline (so all dosages result in upregulation in a bell-shaped response), or they are all below baseline (so all dosages result in downregulation, but in a bell-shaped response), though some straddle the baseline and like the coffee cherry above go from up to down regulation or down to up. There are only a few genes for which the dosage response to chlorogenic acid is linear. This is a startling result.

By (generally) comparing the curves seen with chlorogenic acid versus those from coffee cherry, it is apparent that something is quite different is occurring in the coffee cherry. The chlorogenic acid appears in many cases not to be the ‘dominant’ effect on gene expression. However, there are cases where the chlorogenic is the dominant effect. Of special interest is that as the concentration/dose changes, the response balance shifts and sometimes the chlorogenic acid effect alone is altered to an often ‘opposite’ effect compared to the coffee cherry (see, e.g., FIGS. 11-13, 15, for instance).

In general, with chlorogenic acid, there is overall more activity at the 0.000005% and the 0.0005%, thus highlighting a beneficial dose—and more generally, that less in this case may be more beneficial (or at least more effective) than more.

Without intending to be limited to any one explanation for what is observed in these dosage response analyses, some of the observed effects are likely to be antioxidants that behave as pro-oxidants under certain circumstances, such as low or high concentrations. In addition, in some instances chlorogenic acid (alone, or as a component of the coffee cherry extract) or another component of the coffee cherry extract may be being converted into other related chemical(s)—either by a component of the test biological system, or through equilibrium interconversions (which can be influenced strongly by relative concentration). There could also be other chemical reactions going on. Thus, what is observed is the ‘end result’ of impact on a gene (or set of genes), including any actions that take place somewhere else upstream in a pathway that impacts the specific gene being assayed.

In some instance, receptor sites blocked may be blocked or competitively inhibited (or stimulated) by one or the other of chlorogenic acid or a component in the coffee cherry extract—which can result in complex interactions.

It is also believed that some of the effects observed are due to ‘offsetting penalties’ between different genes, such that when the inducing compound concentration changes the net effect on a gene goes from up to down, or down to up, either as direct effect or some more distant effect in the pathway.

It is also understood that there may be differential effects observed due to inherent differences between the chlorogenic acid and coffee cherry extract used. For instance, the cultured cells might preferentially absorb compounds from COFFEEBERRY® extract, or might preferential absorb some complex of compounds from that mixed extract that are missing from the purified chlorogenic acid preparation; there even may be a synergistic impact from the mixed extract preparation. In addition, the amount of chlorogenic acid used directly is considerably higher than the amount of chlorogenic acid present in the coffee cherry extract; as such, the enriched chlorogenic acid may be trigger effects that are only seen at levels well beyond the levels of coffee cherry extract assayed here.

At the two ‘most active’ concentrations, more than a third of the assayed genes (using Array 1) show significant responses. There is variation in good/bad with the overall dose, as well as the magnitude of effects on specific genes

On the larger microarray (Array 2.0), with coffee cherry extract, more significant changes were observed at the highest concentrations, though there are similar changes at all three concentrations (though the magnitude is different and, as noted, a few genes change from up to down or down to up). It appears the effects on gene expression generally increase in whatever direction they were headed with higher dosages of coffee cherry. Included in this is that gene expression that is becoming ‘less good’ gets more so as the concentration increases. Again, this highlights that the dosage is particularly important.

### Example 17

Dosage Analysis in Human Tissue Samples

This example provides representative methods that can be used to analyze the effects of different dosages of lifespan influencing compounds in a human test system.

In a first embodiment, a formulation containing the test compound (e.g., a composition comprising one or more antioxidant compounds) is applied topically in a serum for instance twice daily in the AM and PM to skin (such as facial skin). Optionally, different subjects in the study are given different dosages of the test compound, and/or different dosage regimens. This elected regimen is followed for 12 weeks, for instance. No other changes in skin care routine are allowed, though daily use of SPF 30 zinc oxide sunscreen is optionally required to enable clear differentiation and recognition of the impact of the text compound.

Biopsies of the subjects' skin are taken using a 3.0 mm punch at pre treatment (to obtain an initiation baseline) and also at 12 weeks after commencing treatment. Biopsies are taken on the upper cheek area both pre and post treatment. A third biopsy is taken at the baseline pre treatment visit from behind the ear in a non sun exposed area. The biopsies are then analyzed to determine changes in gene expression, for instance using one of the custom microarrays described herein. By comparing the different biopsy samples, one can assess changes in gene expression that result from the test compound therapy, as well as changes from environmental/UV light damage (by comparing light exposed to unexposed skin). With multiple subjects to which different dosages of a test compound are applied, dosage response curves can be generated and optimized dosages determined.

In a second embodiment a formulation containing a test compound is taken orally once daily in the AM before meal for 24 weeks. Prior to initiating treatment baseline blood samples and skin biopsies are taken for analysis with a focused microarray, such as one of the custom microarrays provided herein. These samples are repeated at 24 weeks and also analyzed with the same methodology. By comparing gene expression patters from the different samples, one can assess changes in gene expression that result from the test compound therapy. With multiple subjects to which different dosages of a test compound are applied, dosage response curves can be generated and optimized dosages determined.

With these and similar methods (an optionally in combination with or following cell-based microarray analyses), one can characterize the biological effect and effectiveness of, for instance different plant preparations (peel vs. bean or seed vs. pulp vs. stem vs. bark vs. leaves and so forth), preparations form different plants (such as plants listed herein), various concentrations or mixtures or methods of preparing plant extracts, specific components from naturally occurring extracts, and so forth.

### Example 18

### Mechanism of Action for the Application of Sufficient Quantities of Idebenone to Alter the Longevity of Cells

Cells under oxidative stress have a tendency to “stall” electrons around Complex I in the electron transport system which in turn causes damage to the cell. If the electron transport system cannot move the electrons past Complex I, a feedback loop of further ROS generation may occur causing further damage. One of the mechanisms of action of the idebenone compound and its electron derivatives which transfer electrons is the ability to take the electrons and to bypass Complex I transferring them into Complex III further “downstream” and eliminating the tendency for a “bottleneck” at Complex I and an increase in ROS production. The circumnavigation of Complex I increases the efficiency of the mitochondrial respiration and decreases the production/accumulation of ROS and modulating the cell toward an increased lifespan.

### Example 19

### Use of a Modulating Compound in a Topical Composition for Anti-Aging

A stable topical cream formulation containing a set amount of coffee cherry extract (e.g., 0.05%, 0.1%, 0.15%, 0.2%, 0.25%, 0.3%, 0.5%, 0.75%, 1%, 2%) may be applied twice daily to the skin of the face, chest, forearms and hands which are exposed to UV sunlight. The active modulating compounds penetrate the outer epidermal layer of the skin where keratinocytes reside and enter the dermal layer where fibroblasts and many other skin cells reside and the UV light is absorbed into these cells. The cells are environmentally injured by UV light to which these skin areas are exposed. Some of the cells are mildly to moderately injured and various degrees of direct DNA damage is produced in these cells as well as increase in ROS in the cell as well as mitochondrial and membrane injury. Some of the cells are ‘sunburned’ and so severely injured that they will proceed to undergo apoptosis and subsequently these cells will die.

The cells which have been exposed to a sufficient amount of the coffee cherry extract or modulating compounds will have DNA damage either prevented or repaired by mechanisms discussed previously. One or more of the telomere maintenance genes will have their activity modulated to protect and defend or even repair the structural integrity of the telomere on DNA within the nucleus and/or the mitochondrial DNA. The gene expression changes produced by the UV injury may be neutralized or countermanded or alternative repair pathways as described earlier may also be activated or a combination of both activities.

ROS within the mitochondria may also be neutralized or diminished in activity so that the cell injury is either prevented or diminished or repaired. In particular hydroxyl radicals, hydrogen peroxide and reactive nitrogen species may be so affected.

The mitochondrial DNA polymerase enzyme Pol gamma may have its expression level modulated to facilitate repair to mitochondrial DNA.

The modulating compounds may also signal for the biogenesis and production of new mitochondria as well as improving or protecting the respiratory efficiency of the mitochondria by quenching ROS.

The apoptosis process which was being initiated may also be stopped thus preventing cell death.

Thus the lifespan of various cells types and specific cells within the skin may have their lifespan prolonged by protecting the telomere structure or by preventing oxidative stress damage by the ROS or even by preventing cell death via apoptosis. The functional capacity or efficiency of the mitochondria may also be improved either directly or indirectly through increasing the actual number of mitochondria. Mitochondrial biogenesis or increase in number of mitochondria may be produced when the modulating compound activates or increases the activity or expression level of genes which increase mitochondrial numbers such as the gene PGC-1alpha.

Alternate pathways exist for maintaining the telomere structure and these may be activated instead of or in addition to the traditional maintenance pathways.

The net effect of these various pathways and mechanisms of action by the modulating compounds is to allow the structure and function of the skin to maintain a healthier and younger state which in turn allows the skin to maintain a more youthful appearance and delay or minimize premature UV photoaging which is typically manifest by the appearance of fine lines, wrinkles, uneven pigmentation, loss of skin radiance, loss of skin elasticity and tone, skin sagging, reduced blood circulation and often slower wound healing as well as various other signs of premature photoaging. Thus, the topical formulation helps maintain the youthful appearance and functions as an anti-aging topical formulation.

### Example 20

### Combination of Modulating Agents with a Sunscreen Formulation into a Topical Skin Care Lotion

A formulation of 0.1% idebenone in combination with a physical sunscreen zinc oxide and the coffee cherry extract acids at 0.05% in a stable topical lotion is applied to the skin prior to engaging in outdoor sports activities on a bright sunny day. This formulation is used once to twice daily on an regular basis to allow the accumulation of the modulating agents into the skin in a more or less steady state or reservoir effect. The acute sun exposure and activities allow a certain portion of the UV light of all wavelengths to enter the skin. The modulating compounds help protect and defend the skin cells from the UV light injury from the environment. The gene expression changes describe in prior examples illustrate that until the UV exposure occurs that these modulating agents have no effect on the expression levels of many genes and it is only after acute exposure to the UV light that various gene expression modulations begin to occur setting in motion the various protective and repair mechanisms within the skin cells that preserve normal healthy cell function and protect and extend the lifespan of at least some of the cells relative to what would have occurred had the modulating agents not been included in the skin care lotion. The response to the UV light depends on the amount of UV light injury and also to some degree to the proportion of UVB versus UVA1 light which contact the cells since the gene response and DNA damage pattern is different for these different wavelengths of UV light. This mixture of compounds includes an antioxidant idebenone which more specifically targets and protects mitochondria since it is a derivative of the naturally occurring ubiquinone which serve a vital role in mitochondrial function and electron transport. As described earlier idebenone is a smaller molecule thus allowing better penetration into the skin and also it has more potent antioxidant activity than ubiquinone as well.

The antioxidants in the coffee cherry extract and its effects impact the mitochondria less specifically and target various cellular pathways in the cell in general. Thus the coffee cherry extract helps to quench a different mix of ROS and their pathways as well as having a different pattern of gene expression modulations for various protective and repair processes. The differential expression of genes between the idebenone and the coffee cherry extract are seen in prior examples. Also it is noteworthy that some of these gene expression patterns only show a modulation effect on gene expression after the UV exposure occurs and thus show the ability of the modulating compounds to behave in a quiescent manner until oxidative stress or DNA damage or other cellular injury occurs.

### Example 21

### Use of Oral Supplements Containing the Modulating Compounds to Extend the Lifespan of Companion Animals Such as Cats and Dogs

A sufficient amount of idebenone and/or coffee cherry extract may be included in pet food for long term ingestion or in pet vitamin or nutritional supplement tablets or other formulations. As the animals are exposed to various environmental stresses, diseases, and oxidative stress various diverse cells within their tissues and organs will retain their mitochondrial respiratory efficiency and/or telomeric structure longer than if they had not received the modulating compounds. This is not the same process as caloric restriction or the use of compounds which mimic caloric restriction but they result in extension of what would otherwise have been the lifespan of the pet and typically would extend the healthy lifespan of the animal.

Such a supplement or food containing these compounds may also be combined with a caloric restriction mimic such as resveratrol in sufficient amount so that both the caloric restriction pathways which are known to extend lifespan as well as the modulating compound effects are both activated in such as way as to further extend the lifespan of the pet than would have occurred using only resveratrol to supplement the pet's diet.

### Example 22

### The Use of Modulating Compounds to Enhance or Improve the Efficacy of Cancer Chemotherapeutic Agents

A formulation of lifespan modulating compounds is selected which produces inhibition of the telomere maintenance genes and it is administered in conjunction but not necessarily in direct combination with a chemotherapeutic agent targeting the cancer cells. The ability of the cancerous cells to reverse apoptosis and acute cell death and/or the disruption of telomerase activity which helps to preserve or immortalize the cancer cells creates a significantly higher death rate of the cancer cells thus improving the clinical result of the chemotherapy and also potentially increasing the probability of curing the cancer.

Such a formulation may also be utilized to reduce the amount or concentration of chemotherapeutic agent needed to effectively treat the cancer thus reducing the risk of the side effects and adverse events produced by the chemotherapeutic agent.

Yet another possibility is to utilize a combination of lifespan modulating agents so that the above described events occur, but also so that healthy non cancerous cells can be additionally protected from lifespan shortening effects of the chemotherapy or radiation. This is differential modulation wherein the immortalized cancer cells lines are basically made more mortal and susceptible to the cancer therapy while the non cancerous cells have their ability to protect, defend and repair damage from the cancer therapy enhanced.

### Example 23

### Use of Lifespan Modulating Agents to Protect or Extend the Lifespan of Acutely Injured Cells

Idebenone 0.05% incorporated into an aerosol inhaler may be used to treat acute pulmonary injuries. For example a fireman who is suffering from acute smoke inhalation injury to his or her lungs or who has an acute injury from exposure to an environmental hazardous material such as inhaling a toxic gas such as chlorine may repeatedly use an inhaler or nebulizer to help to prevent apoptosis and cell death of vital pulmonary tissues by modulating the gene expression of cells which controls apoptosis. Continued use can help to protect or repair telomere structure so that cells do not have their lifespan shortened so that the lungs as an organ do not as the patient ages suffer premature aging and contribute or directly cause reduced lifespan of the entire organism or patient. The inflammatory pathways which if activated in either an acute or chronic manner may produce DNA damage, mitochondrial inefficiency may also be modulated extending the lifespan of the cells and organ. Stimulation of the biogenesis of additional mitochondria may also help to offset the cells which die thus extending the functional lifespan or the efficiency of the surviving cells. Another example is the exposure of a complex mix of toxic chemicals to rescue workers at the World Trade Center disaster of September 11 and the development a few years later of severe disability from delayed onset of pulmonary fibrosis and other pulmonary problems caused by the inhaled exposure which may have been mitigated by the use of inhaled modulating agents.

### Example 24

### Extending the Lifespan of Plants

Commercially poinsettias and orchids are among the major commercial pot plant and flower crops. It is useful to have uniformity of plant size, flower color, and other physical characteristics as well as flowering time and plant vigor or health so that a uniform crop may be produced and so that the plants may all be given the same culture. Cloning plants has become a major commercial enterprise so that all the plants produced are identical copies of each other. These plants are originally generated from sterile tissue culture in laboratories and eventually the cells lines become senescent and the commercial production may be halted or mutations may enter the cloning process which would produce deformed plants or flowers of no commercial value. Coffee cherry extracts in the coffee plant itself function to protect the plants from various environmental injuries and stress and disease injury. The tissue culture media for this plant cloning process may have one or a combination of modulating agents incorporated into the media to help to extend the lifespan of the cell culture and thus allow longer commercial production of the cloned plant. Reducing the possibility of DNA damage and mutations in the plants is also a potential benefit. Another potential benefit is to help to prevent or repair damage to the plants which might be produced by pesticides or fungicides which are used to treat diseases in these plants once grown out of tissue culture since flower deformities may result from DNA damage to the plants.

### Example 25

### Use of Modulating Agent in Ophthalmic Eye Drop Solution

An ophthalmic preparation containing 0.01% coffee cherry extract is utilized as a preventive therapy for cataracts. Cataracts are thought to be caused in part by environmental damage such as UV light and/or free radical/ROS damage. Protecting or repairing such damage before permanent structural protein changes that lead to cataracts occur may delay the onset or even prevent cataracts.

### Example 26

### Use of Modulating Compounds to Delay the Onset of Gray or White Hair

The graying or turning white hair is a sign of aging. There is widespread variation in the age of onset of gray or white hair. The pigment in hair is produced by pigment producing cells called melanocytes. The melanocytes are replenished by stem cells. It is believed that the death of melanocyte stem cells associated with the hair follicle and hair bulge lead to the development of gray or white hair. A topical formulation to apply to the scalp composed of 1.5% coffee cherry extract and 0.5% idebenone (with optional other ingredients) may be utilized to extend the lifespan of either the melanocyte cells or the stem cells which produce new melanocyte cells.

By prolonging their lifespan the natural color of the hair is preserved until an older age than would have otherwise naturally occurred thus delaying or preventing the onset of gray or white hair. Various mechanisms of action may occur since the hair is subjected to a variety of environmental insults and injuries ranging from UV light, hair care products, hair dye processes, straightening processes, heat from hair dryers, etc. These may contribute to damage to the DNA and telomere maintenance genes or to the mitochondria or both. Either or both the melanocytes and/or the stem cells may be affected. Hair thinning or hair loss allows the scalp to have increased UV exposure which also may accelerate premature aging and hair color change or loss of hair color.

### Example 27

### Organ Transplant Applications

Organs being prepared for transport and subsequent transplantation may before, during or after or combination of these time periods be perfused with a solution containing coffee cherry extract alone or in combination with green tea polyphenols to modulate the gene expression for telomere structure maintenance and/or for modulating the gene expression which controls mitochondrial biogenesis. While there is an important benefit for preventing or diminishing or delaying the onset of apoptosis and viability of the cells and the organ itself thus prolonging the time available from organ harvest to transplantation as well as possibly improving the ability of the organ to survive transplant and/or subsequent anti rejection therapy, an important function is to modulate the telomere structure maintenance and/or mitochondria biogenesis. This may be particularly important when an organ is being transplanted from an older donor into a significantly younger donor so that the lifespan of the organ itself is extended beyond what it would be if it were untreated.

### Example 28

### Extending the Lifespan of Autologous Grafts

Autologous human skin fibroblasts from tissue cell culture are injected into wrinkles and acne scars on the face of the person from which the skin cells were harvested weeks earlier and cultured in vitro before being returned to the person's body. The cell culture had 0.005% idebenone added to their final culture transport media before being frozen for transport to the doctor's office for re-implantation into the person's face. The freeze/thaw cycle and transport as well as the injection process trauma injure or kill a percentage of these autologous fibroblast cells. By utilizing the modulating compound the cell death rate due to apoptosis and also the other injuries is reduced allowing a better transplant success rate. After injection the improved status of the mitochondria allows better cell function in producing structural skin proteins such as collagen which in turn produces a greater reduction in the severity of wrinkles and/or acne scars.

### Example 29

### Demonstrating the Polyphenol Protective Effect on Telomerase Expression Versus UVB Radiation

The antioxidant effect of polyphenols (found in green tea and other sources) is well illustrated in the literature and shows impressive anti-sunburn cell capabilities. What has not been described is the effect addition of polyphenols to UV irradiated cells has on the gene expression profiles of the cells. Specifically the cells in our experimentation (36 y.o. human skin fibroblasts) have demonstrated a 1.6 fold increase in the gene responsible for telomerase activity in non irradiated cells given the polyphenol compounds, and a 1.7 fold increase in irradiated cells given polyphenols when compared to irradiated control cells. This demonstrates that either through direct binding to gene promoter sites, or through second messenger systems in the cellular environment triggered by the ROS reduction/antioxidant effect or some other mechanism(s). These polyphenol compounds trigger the cell to produce more telomerase which protects the DNA and telomere structural integrity or telomere length resulting in a potentially increased lifespan for the cells.

### Example 30

### Lifespan Regulating Compounds Demonstrate Increased Production of PARP1 and TERT Gene Signals in Cells Exposed to UVB Radiation

UVB radiation has been shown to affect a downregulation of TERT and PARP1 in cells so exposed, and this was evidenced in the experiments contained in prior examples. In these experiments the cells exposed to UVB and not treated with any lifespan modulating compounds demonstrated a reduction in the gene expression (and thus indicative of shortened/damaged lifespan) of TERT by 4.6 fold and in PARP1 (a gene involved in DNA repair and apoptosis) a 5.1 fold reduction. Cells of the same age and from the same cell line, when exposed to various lifespan modulating compounds (those tested in these experiments included, polyphenols, coffee cherry and idebenone) demonstrated an UPREGULATION of those same genes in some cases 11 fold for PARP1 and 12 fold for TERT when compared to UVB exposed control cells (for the exact numbers and to view the similar effect on TERT on even 50 year old cells tested, see Example 6 above) indicating an increase in the lifespan and greater repair mechanisms in action for the telomere length and associated structures.

### Example 31

### Use of Modulating Compounds in Conjunction with Other Anti Aging Skin Care Products

A skin care cream containing 0.5% coffee cherry extract and 0.1% each of the antioxidants vitamin E, vitamin C, superoxide dismutase, phloretin, kinetin, alpha lipoic acid, coenzyme Q10, green tea and grape seed extracts, along with appropriate other ingredients to produce a stable formulation, is applied once or twice daily to areas of (prematurely) aging skin on the face, neck, chest, hands and other body areas to achieve two primary benefits. The first benefit is to prevent or delay aging and the second benefit is to repair or reverse existing premature aging of skin cells and related tissues so that the longevity and vitality of these cells is extended and also the appearance of the skin is maintained in a more youthful state.

### Example 32

### Manufacture of a Lifespan-Influencing Array

While representative lifespan-influencing gene arrays are described herein, other arrays can be constructed using art recognized techniques (such as those described or referenced herein), but with sets of genes that are defined based on the research described herein. Thus, additional arrays are contemplated that contain at least some of the genes listed herein in DATA TABLE 7 and/or custom Array 2.

Arrays can be manufactured using art-recognized techniques, including for instance custom array services that are available commercially.

### Example 33

### Use of Lifespan-Influencing Gene Arrays

With the provision herein of specific life-span influencing gene arrays (that is, sets of genes that can be used on arrays), as well as guidance for selecting genes from those discussed herein to form additional arrays, there are now enabled myriad methods of using these arrays.

Merely by way of example, the arrays provided herein can be used to: characterize the lifespan influencing characteristics of test compounds, experimental or known drugs, extracts or enriched fractions thereof or individual components found therein, specific concentrations of such (applied to cells, tissues, organs, or whole organisms—from which a genetic sample is then obtained for the array analysis); characterize the effects of any lifespan influencing substance (such as the compounds and compositions discussed herein) on different cell types (e.g., keratinocytes, melanocytes, liver, cardiac cells, brain cells, muscle cells, cells from blood, and so forth), different animals or other organisms, cells/tissues/organs/organisms of different ages than characterized herein, cells/tissues/organs/organisms under specific stresses (e.g., smoking, hypoxia, infection or other disease, injury, environmental toxin exposure, radiation exposure, different nutritional regimens, undergoing treatments with known or experimental drugs, and so forth); characterize the effects of lifespan influencing substance(s) when applied via a different route than detailed herein; and so forth.

Also contemplated are uses of the arrays to analyze biological samples with regard to longevity/healthy longevity/lifespan separate from the lifespan influencing compositions discussed herein. For instance, the provided arrays can be used to characterize changes in (longevity or lifespan-related) gene expression due to aging (e.g., by testing samples from subjects of different ages, or from the same subject at different times), environmental exposure (e.g., by testing samples from subjects exposed to known or suspected toxins or other environmental conditions), chemical or radiation exposure, disease (including for instance acute or chronic diseases, genetic diseases, infectious diseases, and so forth), dietary or wellness programs (e.g., to evaluate the effectiveness of a selected program), and myriad other uses that will now be recognized in view of the teachings provide herein.

The provided arrays are also useful in epidemiology studies, for instance to look at disparities of health care, differences in geography, people groups, diet, and so forth. Assays of the expression of lifespan related genes can be used to test subjects periodically to determine (like an ‘early warning’ system) if something may be ‘going wrong’ in critical lifespan-involved system (such as telomere maintenance, mitochondrial respiration or biogenesis, and so forth). The arrays could be used as diagnostic tools as well.

The actual methods of assaying the array are conventional, and one of ordinary skill in the art will understand how to prepare and label nucleic acid molecules to be applied to “probe” the arrays.

In view of the many possible embodiments to which the principles of the disclosed invention may be applied, it will be recognized that the illustrated embodiments are only preferred examples of the invention and should not be taken as limiting the scope of the invention. Rather, the scope of the invention is defined by the following claims, including any equivalents thereof. I therefore claim as my invention all that comes within the scope and spirit of these claims.

DATA TABLE 3 (in 15 parts)
Gene expression profiles related to aging, lifespan and telomerase length
maintenance of cells contacted with coffee cherry extract and idebenone
36 y.o. Coffee cherry Alone Continuous Incubation

| | | | | | | | | |
|---|---|---|---|---|---|---|---|---|
| PARP1 | 0.4814 | −1.82 | COX1 | 0.4089 | −1.08 | KRAS | 0.9551 | −1.05 |
| IGF1 | 0.1455 | 3.96 | HPRT1 | 0.8224 | −1.16 | SOD1 | 0.7337 | −1.24 |
| SHC1 | 0.4289 | −3.17 | APOE | 0.6921 | 1.22 | HSPA1L | 0.8281 | −1.20 |
| IL1A | 0.2787 | 1.66 | TERT | 0.0946 | 1.91 | RTC | 0.6814 | −1.64 |
| NADSYN1 | 0.4003 | −2.02 | CYP19A1 | 0.0464 | 1.91 | ACE | 0.6813 | −1.43 |
| GAPDH | 0.4905 | −2.02 | TNF | 0.5853 | 1.35 | TP53 | 0.7248 | −1.48 |
| PARP2 | 0.8527 | −1.20 | PTGS2 | 0.9244 | −1.05 | SOD2 | 0.3321 | −1.76 |
| IGF2 | 0.6898 | 1.10 | HIGX1A | 0.7522 | −1.43 | CASP9 | 0.9592 | 1.02 |
| IFI44 | 0.7012 | 1.18 | HLA-DRA | 0.0843 | 2.52 | CCL4L1 | 0.4603 | 1.31 |
| IL6 | 0.2565 | 1.85 | TEP1 | 0.2991 | −1.95 | PPC | 0.3848 | 1.45 |
| BAX | 0.4215 | −5.15 | BCL2 | 0.6078 | −1.59 | GH1 | 0.2197 | 1.66 |
| ACTB | 0.7831 | −1.33 | HSPA1A | 0.7292 | −1.12 | MAPK14 | 0.5186 | −1.89 |
| PPARG | 0.3948 | 1.78 | HSPA1B | 0.4570 | −7.54 | NOS2A | 0.9857 | −1.01 |
| CLK1 | 0.7581 | −1.20 | RTC | 0.6795 | −1.64 | CASP2 | 0.5059 | −2.96 |
| CREBBP | 0.3673 | −1.24 | DDC | 0.1751 | 2.44 | NFKB1 | 0.6253 | −1.70 |
| IL10 | 0.9829 | 1.02 | SIRT1 | 0.6353 | −1.59 | PPC | 0.4365 | 1.40 |

36 y.o. vs. 50 y.o. Coffee cherry Alone Continuous Incubation

| | | | | | | | | |
|---|---|---|---|---|---|---|---|---|
| PARP1 | 0.7088 | −2.34 | COX1 | 0.2692 | −2.18 | KRAS | 0.6978 | −1.65 |
| IGF1 | 0.4113 | −1.83 | HPRT1 | 0.4447 | 1.60 | SOD1 | 0.8432 | 1.13 |
| SHC1 | 0.7596 | −2.18 | APOE | 0.5618 | −1.30 | HSPA1L | 0.5569 | 1.77 |
| IL1A | 0.7448 | −1.13 | TERT | 0.9540 | −1.02 | RTC | 0.9692 | −1.05 |
| NADSYN1 | 0.7531 | −1.71 | CYP19A1 | 0.1525 | −1.71 | ACE | 0.7029 | −2.03 |
| GAPDH | 0.4732 | 2.59 | TNF | 0.4124 | 1.71 | TP53 | 0.7585 | −1.83 |
| PARP2 | 0.4247 | 2.26 | PTGS2 | 0.1318 | 2.69 | SOD2 | 0.6342 | −1.60 |
| IGF2 | 0.5827 | −1.17 | HIGX1A | 0.5711 | 1.97 | CASP9 | 0.6982 | −1.34 |
| IFI44 | 0.3952 | −1.71 | HLA-DRA | 0.9434 | 1.02 | CCL4L1 | 0.9694 | −1.02 |
| IL6 | 0.7058 | 1.25 | TEP1 | 0.9810 | −1.02 | PPC | 0.1526 | −1.83 |
| BAX | 0.8013 | −2.69 | BCL2 | 0.9670 | −1.05 | GH1 | 0.9535 | 1.02 |
| ACTB | 0.8099 | 1.49 | HSPA1A | 0.5977 | 1.05 | MAPK14 | 0.5895 | −3.08 |
| PPARG | 0.3184 | −1.97 | HSPA1B | 0.9280 | 1.34 | NOS2A | 0.8808 | 1.09 |
| CLK1 | 0.7771 | −1.54 | RTC | 0.9665 | 1.05 | CASP2 | 0.8921 | −1.34 |
| CREBBP | 0.2808 | 1.65 | DDC | 0.1346 | −1.77 | NFKB1 | 0.3718 | 2.98 |
| IL10 | 0.2016 | 4.52 | SIRT1 | 0.8773 | −1.25 | PPC | 0.1994 | −1.90 |

50 y.o. Coffee cherry Alone Continuous Incubation

| | | | | | | | | |
|---|---|---|---|---|---|---|---|---|
| PARP1 | N/A | 2.04 | COX1 | 0.8702 | −1.07 | KRAS | 0.3597 | 45.39 |
| IGF1 | N/A | 1.44 | HPRT1 | 0.2764 | 2.56 | SOD1 | 0.4950 | 1.42 |
| SHC1 | N/A | 2.11 | APOE | 0.0011 | 1.69 | HSPA1L | 0.5542 | 1.42 |
| IL1A | N/A | 1.39 | TERT | 0.3249 | 1.57 | RTC | 0.9617 | 1.04 |
| NADSYN1 | N/A | 1.21 | CYP19A1 | 0.4746 | 1.28 | ACE | 0.7918 | −1.46 |
| GAPDH | N/A | 1.39 | TNF | 0.3249 | 1.57 | TP53 | 0.6977 | −1.93 |
| PARP2 | 0.7364 | 1.23 | PTGS2 | 0.5567 | 1.52 | SOD2 | 0.9635 | 1.04 |
| IGF2 | 0.6104 | 1.28 | HIGX1A | 0.3581 | 1.52 | CASP9 | 0.6203 | 1.47 |
| IFI44 | 0.1906 | 2.30 | HLA-DRA | 0.2931 | 1.57 | CCL4L1 | 0.3399 | 1.42 |
| IL6 | 0.5548 | 1.69 | TEP1 | 0.7089 | −1.23 | PPC | 0.8188 | 1.11 |
| BAX | 0.5178 | −9.16 | BCL2 | 0.6384 | −1.46 | GH1 | 0.1373 | 1.94 |
| ACTB | 0.9569 | −1.07 | HSPA1A | 0.5183 | −9.49 | MAPK14 | 0.6689 | −2.06 |
| PPARG | 0.5834 | 1.47 | HSPA1B | 0.5688 | −4.42 | NOS2A | 0.7032 | 1.23 |
| CLK1 | 0.9345 | −1.11 | RTC | 0.5407 | −2.45 | CASP2 | 0.6704 | −1.86 |
| CREBBP | 0.2776 | 1.69 | DDC | 0.2020 | 2.08 | NFKB1 | 0.1606 | 2.15 |
| IL10 | 0.2524 | 3.04 | SIRT1 | 0.9361 | 1.08 | PPC | 0.5131 | 1.32 |

36 y.o. Coffee cherry + UVB 4 hr Incubation

| | | | | | | | | |
|---|---|---|---|---|---|---|---|---|
| PARP1 | 0.3212 | 2.20 | IL1A | 0.8620 | 1.31 | PARP2 | 0.3327 | 3.33 |
| IGF1 | 0.2417 | 3.82 | NADSYN1 | 0.0140 | 2.61 | IGF2 | 0.7051 | −1.08 |
| SHC1 | 0.7672 | 1.18 | GAPDH | 0.5269 | −2.09 | IFI44 | 0.1756 | 5.60 |
| IL6 | 0.6272 | 1.40 | PTGS2 | 0.9749 | −1.05 | RTC | 0.2333 | 2.61 |
| BAX | 0.3685 | 1.55 | HIGX1A | 0.8108 | −1.20 | ACE | 0.6040 | 1.40 |
| ACTB | 0.9098 | −1.08 | HLA-DRA | 0.4033 | 5.22 | TP53 | 0.5914 | 1.40 |
| PPARG | 0.6320 | 1.35 | TEP1 | 0.6207 | −1.20 | SOD2 | 0.4105 | 1.72 |
| CLK1 | 0.4795 | 1.55 | BCL2 | 0.2860 | 9.75 | CASP9 | 0.1491 | 2.20 |
| CREBBP | 0.4141 | −1.24 | HSPA1A | 0.9074 | −1.12 | CCL4L1 | 0.4384 | 6.00 |
| IL10 | 0.7229 | 1.78 | HSPA1B | 0.8677 | 1.22 | PPC | 0.3529 | 1.55 |
| COX1 | 0.5438 | −1.95 | RTC | 0.0812 | 3.57 | GH1 | 0.6414 | 1.66 |
| HPRT1 | 0.9693 | 1.06 | DDC | 0.4726 | 5.22 | MAPK14 | 0.6002 | 1.22 |
| APOE | 0.6124 | −1.53 | SIRT1 | 0.3009 | 2.05 | NOS2A | 0.3323 | 9.42 |
| TERT | 0.0034 | 8.20 | KRAS | 0.6095 | 1.18 | CASP2 | 0.9547 | −1.08 |
| CYP19A1 | 0.3960 | 8.78 | SOD1 | 0.5843 | −1.95 | NFKB1 | 0.9092 | −1.12 |
| TNF | 0.4982 | −2.32 | HSPA1L | 0.5592 | −1.70 | PPC | 0.3921 | 1.31 |

36 y.o vs. 50 y.o. Coffee cherry + UVB 4 hr Incubation

| | | | | | | | | |
|---|---|---|---|---|---|---|---|---|
| PARP1 | 0.3328 | 2.35 | COX1 | 0.6272 | 1.72 | KRAS | 0.8901 | 1.10 |
| IGF1 | 0.7982 | −1.12 | HPRT1 | 0.3097 | 4.38 | SOD1 | 0.3349 | 3.69 |
| SHC1 | 0.3588 | −1.70 | APOE | 0.9008 | 1.06 | HSPA1L | 0.7806 | −1.34 |
| IL1A | 0.2596 | 2.99 | TERT | 0.6508 | 1.26 | RTC | 0.9643 | 1.02 |
| NADSYN1 | 0.9759 | −1.01 | CYP19A1 | 0.5320 | −1.76 | ACE | 0.5537 | 1.55 |
| GAPDH | 0.2485 | 4.54 | TNF | 0.2392 | 4.54 | TP53 | 0.5255 | −1.29 |
| PARP2 | 0.9204 | 1.06 | PTGS2 | 0.2518 | 5.04 | SOD2 | 0.1456 | −2.77 |
| IGF2 | 0.2860 | −1.25 | HIGX1A | 0.2784 | 2.19 | CASP9 | 0.6621 | 1.17 |
| IFI44 | 0.3656 | −3.07 | HLA-DRA | 0.8871 | −1.08 | CCL4L1 | 0.5735 | 1.66 |
| IL6 | 0.2068 | 1.72 | TEP1 | 0.1518 | 1.98 | PPC | 0.4166 | −1.25 |
| BAX | 0.6667 | 1.26 | BCL2 | 0.4683 | −1.89 | GH1 | 0.2641 | 1.98 |
| ACTB | 0.6502 | −1.29 | HSPA1A | 0.2294 | 3.82 | MAPK14 | 0.0586 | 1.40 |
| PPARG | 0.5024 | 1.60 | HSPA1B | 0.3570 | 2.43 | NOS2A | 0.7580 | −1.43 |
| CLK1 | 0.8118 | −1.08 | RTC | 0.9821 | 1.02 | CASP2 | 0.3684 | 2.89 |
| CREBBP | 0.5484 | 1.45 | DDC | 0.5663 | −1.48 | NFKB1 | 0.7227 | 1.66 |
| IL10 | 0.8347 | 1.26 | SIRT1 | 0.8380 | −1.12 | PPC | 0.1633 | −1.82 |

50 y.o. Coffee cherry + UVB 4 hr Incubation

| | | | | | | | | |
|---|---|---|---|---|---|---|---|---|
| PARP1 | 0.5088 | 2.54 | COX1 | 0.4146 | 1.36 | KRAS | 0.8274 | 1.22 |
| IGF1 | 0.1262 | 3.71 | HPRT1 | 0.0669 | 6.03 | SOD1 | 0.3555 | 1.46 |
| SHC1 | 0.8188 | 1.18 | APOE | 0.3910 | 1.36 | HSPA1L | 0.7302 | −1.69 |
| IL1A | 0.2012 | 3.23 | TERT | 0.0315 | 10.14 | RTC | 0.4629 | 29.70 |
| NADSYN1 | 0.7096 | 1.22 | CYP19A1 | 0.3347 | 2.91 | ACE | 0.7071 | 1.27 |
| GAPDH | 0.2349 | 1.36 | TNF | 0.0617 | 2.29 | TP53 | 0.0506 | 1.56 |
| PARP2 | 0.1806 | 3.84 | PTGS2 | 0.2153 | 2.91 | SOD2 | 0.5778 | −1.33 |
| IGF2 | 0.0012 | −1.20 | HIGX1A | 0.0563 | 2.81 | CASP9 | 0.3960 | 1.99 |
| IFI44 | 0.6249 | 1.36 | HLA-DRA | 0.3899 | 2.29 | CCL4L1 | 0.1444 | 2.91 |
| IL6 | 0.1152 | 3.35 | TEP1 | 0.0431 | 1.99 | PPC | 0.2779 | 1.36 |
| BAX | 0.5659 | 1.36 | BCL2 | 0.4707 | 4.26 | GH1 | 0.1092 | 3.23 |
| ACTB | 0.7274 | −1.33 | HSPA1A | 0.2083 | 1.67 | MAPK14 | 0.2314 | 2.45 |
| PPARG | 0.0858 | 3.84 | HSPA1B | 0.3086 | 2.45 | NOS2A | 0.5392 | 2.21 |
| CLK1 | 0.2767 | 1.62 | RTC | 0.8913 | 1.22 | CASP2 | 0.3498 | 3.84 |
| CREBBP | 0.5234 | 1.46 | DDC | 0.5842 | 2.13 | NFKB1 | 0.7298 | 1.51 |
| IL10 | 0.3245 | 3.59 | SIRT1 | 0.1154 | 2.13 | PPC | 0.4236 | 96.50 |

50 y.o. Coffee cherry + UVB 4 hr Incubation vs. Continuous Incubation

| | | | | | | | | |
|---|---|---|---|---|---|---|---|---|
| PARP1 | 0.3449 | 1.54 | BAX | 0.3500 | 1.54 | CYP19A1 | 0.9869 | 1.02 |
| IGF1 | 0.4739 | −1.60 | ACTB | 0.2413 | 2.25 | TNF | 0.9498 | −1.02 |
| SHC1 | 0.3600 | 1.65 | PPARG | 0.8575 | −1.09 | PTGS2 | 0.8509 | 1.09 |
| IL1A | 0.9618 | −1.02 | CLK1 | 0.4584 | −1.21 | HIGX1A | 0.7309 | 1.17 |
| NADSYN1 | 0.3013 | 1.71 | CREBBP | 0.7023 | 1.25 | HLA-DRA | 0.9038 | −1.06 |
| GAPDH | 0.7145 | 1.09 | IL10 | 0.9793 | −1.02 | TEP1 | 0.2362 | 1.29 |
| PARP2 | 0.8203 | −1.09 | COX1 | 0.4876 | 1.25 | BCL2 | 0.5908 | 1.29 |
| IGF2 | 0.0438 | −1.06 | HPRT1 | 0.2449 | −1.21 | HSPA1A | 0.4169 | −77.57 |
| IFI44 | 0.7997 | 1.17 | APOE | 0.7300 | −1.13 | HSPA1B | 0.5820 | 1.13 |
| IL6 | 0.9645 | 1.02 | TERT | 0.8161 | −1.13 | MAPK14 | 0.8326 | 1.02 |
| RTC | 0.4196 | 2.33 | ACE | 0.4345 | 1.54 | NOS2A | 0.6992 | 1.59 |
| DDC | 0.4967 | −1.66 | TP53 | 0.1623 | 1.21 | CASP2 | 0.0069 | −1.34 |
| SIRT1 | 0.6142 | 1.05 | SOD2 | 0.1719 | 1.77 | NFKB1 | 0.5920 | 1.77 |
| KRAS | 0.4087 | 1.77 | CASP9 | 0.8912 | −1.09 | PPC | 0.3749 | −1.21 |
| SOD1 | 0.7242 | −1.13 | CCL4L1 | 0.7092 | −1.25 | | | |
| HSPA1L | 0.6392 | 1.65 | PPC | 0.9070 | 1.02 | | | |
| RTC | 0.3251 | 1.65 | GH1 | 0.5513 | −1.71 | | | |

36 y.o. Coffee cherry + UVB 4 hr Incubation vs. Continuous Incubation

| | | | | | | | | |
|---|---|---|---|---|---|---|---|---|
| PARP1 | 0.2956 | −2.24 | COX1 | 0.5086 | −2.09 | KRAS | 0.2752 | −1.82 |
| IGF1 | 0.4460 | 1.50 | HPRT1 | 0.4315 | −2.96 | SOD1 | 0.3662 | −3.28 |
| SHC1 | 0.7109 | −1.08 | APOE | 0.8778 | −1.08 | HSPA1L | 0.3163 | −1.20 |
| IL1A | 0.4828 | −1.89 | TERT | 0.1802 | 2.27 | RTC | 0.1173 | −1.82 |
| NADSYN1 | 0.4138 | −1.12 | CYP19A1 | 0.3134 | 1.91 | ACE | 0.4984 | −1.64 |
| GAPDH | 0.3384 | −3.17 | TNF | 0.4614 | −2.32 | TP53 | 0.7013 | 1.22 |
| PARP2 | 0.3644 | 1.72 | PTGS2 | 0.4666 | −2.49 | SOD2 | 0.6794 | 1.22 |
| IGF2 | 0.4035 | 1.18 | HIGX1A | 0.3273 | −1.89 | CASP9 | 0.1933 | −1.38 |
| IFI44 | 0.6960 | 1.45 | HLA-DRA | 0.2767 | 2.12 | CCL4L1 | 0.5236 | −1.64 |
| IL6 | 0.4816 | 1.22 | TEP1 | 0.4054 | −1.43 | PPC | 0.2045 | 1.55 |
| BAX | 0.2836 | −1.64 | BCL2 | 0.5498 | 1.55 | GH1 | 0.5394 | −1.43 |
| ACTB | 0.0528 | 1.31 | HSPA1A | 0.3134 | −2.96 | MAPK14 | 0.2539 | −1.82 |
| PPARG | 0.4845 | −1.48 | HSPA1B | 0.2633 | −3.17 | NOS2A | 0.8703 | 1.10 |
| CLK1 | 0.8614 | 1.06 | RTC | 0.0930 | −2.49 | CASP2 | 0.4165 | −2.58 |
| CREBBP | 0.7821 | 1.06 | DDC | 0.3794 | 1.91 | NFKB1 | 0.3356 | −3.06 |
| IL10 | 0.6248 | 1.66 | SIRT1 | 0.6137 | 1.40 | PPC | 0.4883 | 1.22 |

36 y.o. Coffee cherry + UVB Continuous Incubation

| | | | | | | | | |
|---|---|---|---|---|---|---|---|---|
| PARP1 | 0.0121 | 4.92 | COX1 | 0.5016 | 1.07 | KRAS | 0.1345 | 2.14 |
| IGF1 | 0.4005 | 2.55 | HPRT1 | 0.2981 | 3.14 | SOD1 | 0.0616 | 1.68 |
| SHC1 | 0.6534 | 1.27 | APOE | 0.6873 | −1.41 | HSPA1L | 0.6957 | −1.41 |
| IL1A | 0.5547 | 2.46 | TERT | 0.0814 | 3.61 | RTC | 0.0971 | 4.76 |
| NADSYN1 | 0.0165 | 2.93 | CYP19A1 | 0.5334 | 4.59 | ACE | 0.1036 | 2.30 |
| GAPDH | 0.3207 | 1.52 | TNF | 1.0000 | −1.00 | TP53 | 0.8142 | 1.15 |
| PARP2 | 0.5398 | 1.93 | PTGS2 | 0.4070 | 2.38 | SOD2 | 0.5083 | 1.41 |
| IGF2 | 0.1455 | −1.27 | HIGX1A | 0.4651 | 1.57 | CASP9 | 0.1007 | 3.03 |
| IFI44 | 0.0309 | 3.86 | HLA-DRA | 0.6207 | 2.46 | CCL4L1 | 0.3269 | 9.85 |
| IL6 | 0.8448 | 1.15 | TEP1 | 0.4738 | 1.19 | PPC | 1.0000 | −1.00 |
| BAX | 0.0848 | 2.55 | BCL2 | 0.3366 | 6.28 | GH1 | 0.4457 | 2.38 |
| ACTB | 0.6333 | −1.41 | HSPA1A | 0.1552 | 2.64 | MAPK14 | 0.2449 | 2.22 |
| PPARG | 0.1841 | 2.00 | HSPA1B | 0.2060 | 3.86 | NOS2A | 0.3615 | 8.57 |
| CLK1 | 0.4634 | 1.46 | RTC | 0.0126 | 8.88 | CASP2 | 0.4133 | 2.38 |
| CREBBP | 0.2484 | −1.32 | DDC | 0.6454 | 2.73 | NFKB1 | 0.0057 | 2.73 |
| IL10 | 0.9625 | 1.07 | SIRT1 | 0.3382 | 1.46 | PPC | 0.6513 | 1.07 |

36 y.o vs. 50 y.o. Coffee cherry + UVB Continuous Incubation

| | | | | | | | | |
|---|---|---|---|---|---|---|---|---|
| PARP1 | 0.0007 | 1.61 | COX1 | 0.8397 | 1.03 | SIRT1 | 0.4387 | 1.31 |
| IGF1 | 0.7786 | −1.20 | HPRT1 | 0.4457 | 1.22 | KRAS | 0.8734 | 1.06 |
| SHC1 | 0.4316 | −1.12 | APOE | 0.7360 | −1.16 | SOD1 | 0.9816 | −1.01 |
| IL1A | 0.4048 | 1.56 | TERT | 0.1666 | 2.53 | HSPA1L | 0.8851 | 1.03 |
| NADSYN1 | 0.1637 | 1.51 | CYP19A1 | 0.8888 | 1.10 | RTC | 0.2541 | −1.08 |
| GAPDH | 0.0667 | 1.56 | TNF | 0.1850 | 1.92 | ACE | 0.4675 | 1.45 |
| PARP2 | 0.2090 | 1.67 | PTGS2 | 0.1997 | 2.20 | TP53 | 0.7108 | 1.14 |
| IGF2 | 0.0645 | −1.12 | HIGX1A | 0.4585 | 1.36 | SOD2 | 0.3934 | −1.28 |
| IFI44 | 0.0662 | −1.82 | HLA-DRA | 0.2583 | 1.85 | CASP9 | 0.6690 | −1.28 |
| IL6 | 0.0944 | 2.13 | TEP1 | 0.1210 | 1.79 | CCL4L1 | 0.5946 | −1.24 |
| BAX | 0.4454 | 1.18 | BCL2 | 0.7666 | 1.06 | PPC | 0.2620 | 1.27 |
| ACTB | 0.0211 | 2.28 | HSPA1A | 0.4406 | −60.13 | GH1 | 0.8100 | −1.24 |
| PPARG | 0.9800 | −1.01 | HSPA1B | 0.5153 | −1.16 | MAPK14 | 0.5737 | −1.28 |
| CLK1 | 0.4086 | −1.24 | RTC | 0.6710 | −1.04 | NOS2A | 0.7539 | 1.22 |
| CREBBP | 0.0559 | 1.92 | DDC | 0.7235 | −1.28 | CASP2 | 0.3106 | −1.20 |
| IL10 | 0.2989 | 2.06 | | | | NFKB1 | 0.8805 | −1.04 |
| PPC | 0.0175 | −1.82 | | | | | | |

50 y.o. Coffee cherry + UVB Continuous Incubation

| | | | | | | | | |
|---|---|---|---|---|---|---|---|---|
| PARP1 | 0.3455 | 3.90 | COX1 | 0.1117 | 1.70 | KRAS | 0.3554 | 2.17 |
| IGF1 | 0.2680 | 2.32 | HPRT1 | 0.0855 | 4.98 | SOD1 | 0.5807 | 1.29 |
| SHC1 | 0.3071 | 1.95 | APOE | 0.2885 | 1.20 | HSPA1L | 0.9824 | −1.02 |
| IL1A | 0.1552 | 3.17 | TERT | 0.0080 | 8.97 | RTC | 0.4080 | 49.01 |
| NADSYN1 | 0.1731 | 2.09 | CYP19A1 | 0.2500 | 2.96 | ACE | 0.3526 | 1.95 |
| GAPDH | 0.0606 | 1.48 | TNF | 0.0397 | 2.24 | TP53 | 0.0177 | 1.89 |
| PARP2 | 0.1708 | 3.52 | PTGS2 | 0.2185 | 3.17 | SOD2 | 0.5165 | 1.33 |
| IGF2 | 0.0022 | −1.26 | HIGX1A | 0.0613 | 3.28 | CASP9 | 0.4991 | 1.82 |
| IFI44 | 0.0837 | 1.59 | HLA-DRA | 0.3995 | 2.17 | CCL4L1 | 0.0890 | 2.32 |
| IL6 | 0.0816 | 3.40 | TEP1 | 0.0442 | 2.58 | PPC | 0.2193 | 1.38 |
| BAX | 0.1236 | 2.09 | BCL2 | 0.3981 | 5.52 | GH1 | 0.5088 | 1.89 |
| ACTB | 0.4217 | 1.70 | HSPA1A | 0.4661 | −46.37 | MAPK14 | 0.2237 | 2.49 |
| PPARG | 0.0403 | 3.52 | HSPA1B | 0.2774 | 2.76 | NOS2A | 0.1128 | 3.52 |
| CLK1 | 0.5204 | 1.33 | RTC | 0.4083 | 2.86 | CASP2 | 0.4449 | 2.86 |
| CREBBP | 0.0429 | 1.82 | DDC | 0.8529 | 1.29 | NFKB1 | 0.2461 | 2.67 |
| IL10 | 0.2465 | 3.52 | SIRT1 | 0.1096 | 2.24 | PPC | 0.4399 | 79.62 |

36 y.o. Coffee cherry + UVB vs. idebenone + UVB 4 hr Incubation

| | | | | | | | | |
|---|---|---|---|---|---|---|---|---|
| PARP1 | 0.3738 | −1.95 | COX1 | 0.6052 | −1.75 | KRAS | 0.9176 | −1.08 |
| IGF1 | 0.7364 | −1.08 | HPRT1 | 0.3925 | −3.27 | SOD1 | 0.4279 | −2.75 |
| SHC1 | 0.9693 | −1.01 | APOE | 0.6820 | −1.16 | HSPA1L | 0.2252 | −1.20 |
| IL1A | 0.5064 | −1.95 | TERT | 0.8108 | −1.04 | RTC | 0.8260 | −1.20 |
| NADSYN1 | 0.0967 | 1.18 | CYP19A1 | 0.5033 | 1.27 | ACE | 0.4599 | −1.64 |
| GAPDH | 0.5278 | −2.01 | TNF | 0.6281 | −1.95 | TP53 | 0.3379 | 1.61 |
| PARP2 | 0.8345 | −1.12 | PTGS2 | 0.4148 | −2.75 | SOD2 | 0.3182 | 1.61 |
| IGF2 | 0.4850 | 1.14 | HIGX1A | 0.3573 | −2.31 | CASP9 | 0.2907 | 1.06 |
| IFI44 | 0.4516 | 2.13 | HLA-DRA | 0.6802 | 1.22 | CCL4L1 | 0.5331 | −1.64 |
| IL6 | 0.5969 | 1.10 | TEP1 | 0.3811 | −1.47 | PPC | 0.2736 | 1.45 |
| BAX | 0.8262 | 1.10 | BCL2 | 0.3954 | 2.36 | GH1 | 0.1970 | −2.01 |
| ACTB | 0.0160 | 1.85 | HSPA1A | 0.5520 | −1.88 | MAPK14 | 0.5020 | −1.20 |
| PPARG | 0.4797 | −1.47 | HSPA1B | 0.6446 | −1.64 | NOS2A | 0.0415 | −1.75 |
| CLK1 | 0.8646 | 1.10 | RTC | 0.4411 | −1.33 | CASP2 | 0.7744 | −1.42 |
| CREBBP | 0.7578 | 1.06 | DDC | 0.8447 | 1.10 | NFKB1 | 0.2880 | −3.63 |
| IL10 | 0.8124 | 1.22 | SIRT1 | 0.5315 | 1.45 | PPC | 0.8082 | 1.06 |

36 y.o. Coffee cherry + UVB vs. idebenone + UVB Continuous Incubation

| | | | | | | | | |
|---|---|---|---|---|---|---|---|---|
| PARP1 | 0.4251 | 2.98 | COX1 | 0.8990 | 1.02 | KRAS | 0.7658 | 1.13 |
| IGF1 | 0.5375 | −1.34 | HPRT1 | 0.5432 | 1.71 | SOD1 | 0.4252 | 2.88 |
| SHC1 | 0.3658 | 1.90 | APOE | 0.2934 | −2.11 | HSPA1L | 0.4545 | 3.80 |
| IL1A | 0.7984 | 1.25 | TERT | 0.7044 | −1.21 | RTC | 0.4368 | 3.08 |
| NADSYN1 | 0.3711 | 2.03 | CYP19A1 | 0.7044 | −1.21 | ACE | 0.9690 | −1.02 |
| GAPDH | 0.4512 | 2.78 | TNF | 0.8700 | 1.09 | TP53 | 0.9219 | −1.05 |
| PARP2 | 0.2957 | −1.77 | PTGS2 | 0.6918 | 1.30 | SOD2 | 0.4436 | 1.21 |
| IGF2 | 0.4895 | −1.17 | HIGX1A | 0.6365 | −1.09 | CASP9 | 0.5349 | −1.71 |
| IFI44 | 0.2929 | 1.30 | HLA-DRA | 0.3151 | −1.60 | CCL4L1 | 0.9581 | −1.02 |
| IL6 | 0.5279 | 2.11 | TEP1 | 0.8209 | 1.05 | PPC | 0.3075 | −1.25 |
| BAX | 0.5545 | 1.13 | BCL2 | 0.7120 | −1.13 | GH1 | 0.8821 | 1.09 |
| ACTB | 0.4383 | 2.78 | HSPA1A | 0.5376 | 1.97 | MAPK14 | 0.5219 | 1.34 |
| PPARG | 0.3168 | −1.60 | HSPA1B | 0.4096 | 2.26 | NOS2A | 0.6187 | 2.26 |
| CLK1 | 0.5631 | −1.17 | RTC | 0.5075 | −1.13 | CASP2 | 0.3596 | 2.18 |
| CREBBP | 0.7171 | −1.09 | DDC | 0.3145 | −1.77 | NFKB1 | 0.5887 | 1.17 |
| IL10 | 0.9444 | −1.05 | SIRT1 | 0.7554 | −1.21 | PPC | 0.3043 | −1.13 |

50 y.o. coffee cherry + UVB vs. idebenone + UVB 4 hr Incubation

| | | | | | | | | |
|---|---|---|---|---|---|---|---|---|
| PARP1 | 0.2058 | −1.92 | SHC1 | 0.2510 | −2.06 | NADSYN1 | 0.9031 | 1.08 |
| IGF1 | 0.4047 | 1.47 | IL1A | 0.3339 | 2.16 | GAPDH | 0.5211 | 1.95 |
| PARP2 | 0.6903 | −1.22 | TNF | 0.6726 | 1.20 | ACE | 0.5805 | −1.22 |
| IGF2 | 0.6710 | −1.06 | PTGS2 | 0.1710 | −1.61 | TP53 | 0.3748 | 1.64 |
| IFI44 | 0.3320 | −2.20 | HIGX1A | 0.3717 | 1.42 | SOD2 | 0.5508 | −1.27 |
| IL6 | 0.4824 | 11.39 | HLA-DRA | 0.6591 | −1.22 | CASP9 | 0.6913 | −1.27 |
| BAX | 0.5213 | −1.40 | TEP1 | 0.4657 | −1.31 | CCL4L1 | 0.9055 | −1.06 |
| ACTB | 0.9946 | 1.01 | BCL2 | 0.4728 | −1.40 | PPC | 0.4534 | −1.14 |
| PPARG | 0.8261 | −1.18 | HSPA1A | 0.7803 | −1.06 | GH1 | 0.9073 | 1.04 |
| CLK1 | 0.2809 | 1.24 | HSPA1B | 0.1106 | −1.56 | MAPK14 | 0.2176 | −1.27 |
| CREBBP | 0.8815 | −1.10 | RTC | 0.9771 | −1.03 | NOS2A | 0.6837 | −1.61 |
| IL10 | 0.9001 | −1.14 | DDC | 0.6024 | −1.45 | CASP2 | 0.3598 | 1.53 |
| COX1 | 0.8182 | −1.14 | SIRT1 | 0.5965 | −1.18 | NFKB1 | 0.4643 | −2.20 |
| HPRT1 | 0.9120 | −1.03 | KRAS | 0.4068 | −1.73 | PPC | 0.8868 | 1.04 |
| APOE | 0.9695 | −1.03 | SOD1 | 0.7291 | 1.08 | | | |
| TERT | 0.5749 | 1.38 | HSPA1L | 0.7448 | −1.40 | | | |
| CYP19A1 | 0.6857 | −1.40 | RTC | 0.9900 | 1.01 | | | |

50 y.o. coffee cherry + UVB vs. idebenone + UVB Continuous Incubation

| | | | | | | | | |
|---|---|---|---|---|---|---|---|---|
| PARP1 | 0.3246 | 2.34 | COX1 | 0.4157 | 2.42 | KRAS | 0.9583 | 1.02 |
| IGF1 | 0.6341 | −1.49 | HPRT1 | 0.9176 | −1.05 | SOD1 | 0.7037 | 1.17 |
| SHC1 | 0.8089 | 1.02 | APOE | 0.9181 | 1.02 | HSPA1L | 0.6209 | 2.42 |
| IL1A | 0.6229 | −1.13 | TERT | 0.9542 | −1.02 | RTC | 0.3072 | 3.09 |
| NADSYN1 | 0.3766 | 1.34 | CYP19A1 | 0.6028 | 2.26 | ACE | 0.9617 | 1.02 |
| GAPDH | 0.4509 | 2.04 | TNF | 0.5378 | 1.49 | TP53 | 0.7352 | 1.13 |
| PARP2 | 0.3061 | 1.39 | PTGS2 | 0.8050 | 1.17 | SOD2 | 0.1610 | 1.30 |
| IGF2 | 0.5656 | −1.13 | HIGX1A | 0.5209 | 2.69 | CASP9 | 0.7845 | −1.17 |
| IFI44 | 0.9537 | 1.02 | HLA-DRA | 0.7716 | −1.13 | CCL4L1 | 0.7237 | 1.66 |
| IL6 | 0.6663 | 1.13 | TEP1 | 0.1388 | 1.44 | PPC | 0.1440 | 1.30 |
| BAX | 0.3577 | 2.18 | BCL2 | 0.3898 | 1.44 | GH1 | 0.6058 | −1.59 |
| ACTB | 0.5770 | 1.30 | HSPA1A | 0.4268 | −69.67 | MAPK14 | 0.9450 | 1.02 |
| PPARG | 0.7677 | 1.09 | HSPA1B | 0.2583 | −1.34 | NOS2A | 0.6361 | 1.77 |
| CLK1 | 0.2436 | −1.39 | RTC | 0.2137 | 1.17 | CASP2 | 0.5372 | 2.51 |
| CREBBP | 0.3244 | 1.54 | DDC | 0.7080 | −1.44 | NFKB1 | 0.2938 | −1.39 |
| IL10 | 0.6686 | 1.71 | SIRT1 | 0.5256 | 1.13 | PPC | 0.5039 | −1.09 |

DATA TABLE 4
(in 18 parts): Gene expression profile related to aging,
lifespan and telomerase length maintenance of cells
contacted with green tea polyphenols and idebenone
36y.o. Green tea + UVB Continuous Incubation

| | | | | | | | | |
|---|---|---|---|---|---|---|---|---|
| PARP1 | 0.0145 | 4.42 | COX1 | 0.1845 | −1.11 | KRAS | 0.0450 | 1.56 |
| IGF1 | 0.8155 | −1.24 | HPRT1 | 0.7109 | 1.41 | SOD1 | 0.3759 | 1.19 |
| SHC1 | 0.9516 | 1.03 | APOE | 0.5334 | −1.63 | HSPA1L | 0.2276 | −3.88 |
| IL1A | 0.3111 | −5.30 | TERT | 0.0333 | 1.23 | RTC | 0.0928 | 5.08 |
| NADSYN1 | 0.0075 | 3.35 | CYP19A1 | 0.5361 | −4.45 | ACE | 0.0552 | 2.63 |
| GAPDH | 0.7113 | 1.14 | TNF | 0.0300 | −18.44 | TP53 | 0.7676 | −1.15 |
| PARP2 | 0.1817 | −6.52 | PTGS2 | 0.4727 | −2.01 | SOD2 | 0.5505 | 1.31 |
| IGF2 | 0.5798 | 1.07 | HIGX1A | 0.0225 | −23.51 | CASP9 | 0.1287 | 2.45 |
| IFI44 | 0.0439 | 2.72 | HLA-DRA | 0.2060 | −16.62 | CCL4L1 | 0.7950 | −1.69 |
| IL6 | 0.6050 | −1.47 | TEP1 | 0.6623 | 1.07 | PPC | 0.1940 | 1.86 |
| BAX | 0.0792 | 2.29 | BCL2 | 0.4838 | 3.47 | GH1 | 0.1372 | −8.03 |
| ACTB | 0.4424 | −1.81 | HSPA1A | 0.1962 | 2.06 | MAPK14 | 0.1248 | 2.29 |
| PPARG | 0.3170 | −1.69 | HSPA1B | 0.2438 | 3.47 | NOS2A | 0.3128 | −10.59 |
| CLK1 | 0.3489 | 1.68 | RTC | 0.0132 | 9.16 | CASP2 | 0.4597 | 2.14 |
| CREBBP | 0.0633 | −1.81 | DDC | 0.3061 | −12.17 | NFKB1 | 0.0561 | 2.63 |
| IL10 | 0.4793 | −2.84 | SIRT1 | 0.2663 | 1.27 | PPC | 0.0481 | 2.14 |

50y.o. Green tea + UVB Continuous Incubation

| | | | | | | | | |
|---|---|---|---|---|---|---|---|---|
| PARP1 | 0.4044 | 3.31 | SHC1 | 0.6517 | 1.30 | NADSYN1 | 0.5313 | 1.39 |
| IGF1 | 0.3052 | −1.77 | IL1A | 0.4683 | −2.98 | GAPDH | 0.8026 | −1.25 |
| PARP2 | 0.6234 | −1.77 | TNF | 0.1881 | −6.84 | ACE | 0.5399 | 1.44 |
| IGF2 | 0.5984 | 1.02 | PTGS2 | 0.2132 | 2.88 | TP53 | 0.8824 | −1.13 |
| IFI44 | 0.6281 | 1.21 | HIGX1A | 0.3282 | −5.01 | SOD2 | 0.7162 | −1.30 |
| IL6 | 0.1122 | 2.88 | HLA-DRA | 0.0529 | −23.83 | CASP9 | 0.7039 | 1.34 |
| BAX | 0.4872 | 1.39 | TEP1 | 0.4043 | 1.60 | CCL4L1 | 0.2277 | −3.54 |
| ACTB | 0.3884 | 2.18 | BCL2 | 0.4664 | 4.21 | PPC | 0.1298 | 1.77 |
| PPARG | 0.4462 | 1.49 | HSPA1A | 0.4060 | 1.34 | GH1 | 0.0274 | −8.43 |
| CLK1 | 0.2369 | 1.83 | HSPA1B | 0.2548 | 3.19 | MAPK14 | 0.5988 | 1.49 |
| CREBBP | 0.2162 | 1.30 | RTC | 0.9100 | 1.17 | NOS2A | 0.3353 | −7.59 |
| IL10 | 0.4300 | −2.11 | DDC | 0.1728 | −13.22 | CASP2 | 0.5897 | 2.03 |
| COX1 | 0.2263 | 1.77 | SIRT1 | 0.8732 | 1.05 | NFKB1 | 0.7298 | 1.65 |
| HPRT1 | 0.3858 | 2.18 | KRAS | 0.6797 | 1.39 | PPC | 0.4083 | 117.38 |
| APOE | 0.5984 | 1.02 | SOD1 | 0.3109 | 1.71 | | | |
| TERT | 0.3207 | 1.21 | HSPA1L | 0.7148 | −1.54 | | | |
| CYP19A1 | 0.1489 | −6.17 | RTC | 0.5550 | 14.67 | | | |

36y.o. vs. 50y.o. idebenone + UVB 4 hr Incubation

| | | | | | | | | |
|---|---|---|---|---|---|---|---|---|
| PARP1 | 0.0177 | 2.32 | COX1 | 0.8230 | 1.12 | KRAS | 0.4448 | 1.76 |
| IGF1 | 0.0479 | −1.79 | HPRT1 | 0.2886 | 1.38 | SOD1 | 0.2294 | 1.24 |
| SHC1 | 0.3764 | 1.20 | APOE | 0.9269 | −1.06 | HSPA1L | 0.3328 | −1.14 |
| IL1A | 0.6965 | −1.40 | TERT | 0.6551 | −1.14 | RTC | 0.8464 | −1.18 |
| NADSYN1 | 0.8722 | 1.08 | CYP19A1 | 0.9687 | 1.01 | ACE | 0.0895 | 1.16 |
| GAPDH | 0.8778 | 1.16 | TNF | 0.5039 | 1.95 | TP53 | 0.6273 | −1.31 |
| PARP2 | 0.7420 | 1.16 | PTGS2 | 0.0063 | 2.95 | SOD2 | 0.3084 | −1.35 |
| IGF2 | 0.8595 | −1.03 | HIGX1A | 0.5417 | −1.50 | CASP9 | 0.3753 | 1.58 |
| IFI44 | 0.3414 | 1.53 | HLA-DRA | 0.3606 | 1.38 | CCL4L1 | 0.7969 | 1.08 |
| IL6 | 0.5914 | −6.01 | TEP1 | 0.2487 | 1.76 | PPC | 0.2713 | 1.33 |
| BAX | 0.2084 | 1.95 | BCL2 | 0.3973 | 1.76 | GH1 | 0.7862 | −1.06 |
| ACTB | 0.6924 | 1.43 | HSPA1A | 0.2398 | 2.16 | MAPK14 | 0.2515 | 1.48 |
| PPARG | 0.6944 | 1.29 | HSPA1B | 0.2666 | 2.32 | NOS2A | 0.0408 | −1.56 |
| CLK1 | 0.6873 | −1.22 | RTC | 0.3345 | −1.26 | CASP2 | 0.7096 | 1.33 |
| CREBBP | 0.2296 | 1.70 | DDC | 0.8857 | 1.08 | NFKB1 | 0.9682 | 1.01 |
| IL10 | 0.4968 | 1.76 | SIRT1 | 0.2506 | 1.53 | PPC | 0.1012 | −1.79 |

36y.o. idebenone + UVB 4 hr Incubation

| | | | | | | | | |
|---|---|---|---|---|---|---|---|---|
| PARP1 | 0.0200 | 4.27 | COX1 | 0.2795 | −1.11 | KRAS | 0.7286 | 1.27 |
| IGF1 | 0.2169 | 4.13 | HPRT1 | 0.2608 | 3.47 | SOD1 | 0.1267 | 1.41 |
| SHC1 | 0.7443 | 1.19 | APOE | 0.7129 | −1.32 | HSPA1L | 0.6909 | −1.42 |
| IL1A | 0.5568 | 2.54 | TERT | 0.0024 | 8.54 | RTC | 0.3173 | 3.13 |
| NADSYN1 | 0.0157 | 2.21 | CYP19A1 | 0.4362 | 6.94 | ACE | 0.0188 | 2.29 |
| GAPDH | 0.9147 | −1.04 | TNF | 0.8663 | −1.19 | TP53 | 0.7888 | −1.15 |
| PARP2 | 0.2812 | 3.72 | PTGS2 | 0.3475 | 2.63 | SOD2 | 0.8805 | 1.07 |
| IGF2 | 0.1798 | −1.24 | HIGX1A | 0.4557 | 1.93 | CASP9 | 0.1711 | 2.06 |
| IFI44 | 0.0473 | 2.63 | HLA-DRA | 0.4423 | 4.27 | CCL4L1 | 0.3284 | 9.82 |
| IL6 | 0.7289 | 1.27 | TEP1 | 0.4326 | 1.23 | PPC | 0.8705 | 1.07 |
| BAX | 0.4255 | 1.41 | BCL2 | 0.4573 | 4.13 | GH1 | 0.2961 | 3.35 |
| ACTB | 0.3801 | −2.01 | HSPA1A | 0.4586 | 1.68 | MAPK14 | 0.4266 | 1.46 |
| PPARG | 0.1752 | 1.99 | HSPA1B | 0.5241 | 1.99 | NOS2A | 0.2529 | 16.51 |
| CLK1 | 0.6173 | 1.41 | RTC | 0.0241 | 4.74 | CASP2 | 0.8111 | 1.31 |
| CREBBP | 0.2238 | −1.32 | DDC | 0.4857 | 4.74 | NFKB1 | 0.0150 | 3.24 |
| IL10 | 0.7846 | 1.46 | SIRT1 | 0.1374 | 1.41 | PPC | 0.2179 | 1.23 |

50y.o. idebenone + UVB 4 hr Incubation

| | | | | | | | | |
|---|---|---|---|---|---|---|---|---|
| PARP1 | 0.2058 | 1.92 | IGF2 | 0.6710 | 1.06 | CREBBP | 0.8815 | 1.10 |
| IGF1 | 0.4047 | −1.47 | IFI44 | 0.3320 | 2.20 | IL10 | 0.9001 | 1.14 |
| SHC1 | 0.2510 | 2.06 | IL6 | 0.4824 | −11.39 | COX1 | 0.8182 | 1.14 |
| IL1A | 0.3339 | −2.16 | BAX | 0.5213 | 1.40 | HPRT1 | 0.9120 | 1.03 |
| NADSYN1 | 0.9031 | −1.08 | ACTB | 0.9946 | −1.01 | APOE | 0.9695 | 1.03 |
| GAPDH | 0.5211 | −1.95 | PPARG | 0.8261 | 1.18 | TERT | 0.5749 | −1.38 |
| PARP2 | 0.6903 | 1.22 | CLK1 | 0.2809 | −1.24 | CYP19A1 | 0.6857 | 1.40 |
| TNF | 0.6726 | −1.20 | DDC | 0.6024 | 1.45 | CASP9 | 0.6913 | 1.27 |
| PTGS2 | 0.1710 | 1.61 | SIRT1 | 0.5965 | 1.18 | CCL4L1 | 0.9055 | 1.06 |
| HIGX1A | 0.3717 | −1.42 | KRAS | 0.4068 | 1.73 | PPC | 0.4534 | 1.14 |
| HLA-DRA | 0.6591 | 1.22 | SOD1 | 0.7291 | −1.08 | GH1 | 0.9073 | −1.04 |
| TEP1 | 0.4657 | 1.31 | HSPA1L | 0.7448 | 1.40 | MAPK14 | 0.2176 | 1.27 |
| BCL2 | 0.4728 | 1.40 | RTC | 0.9900 | −1.01 | NOS2A | 0.6837 | 1.61 |
| HSPA1A | 0.7803 | 1.06 | ACE | 0.5805 | 1.22 | CASP2 | 0.3598 | −1.53 |
| HSPA1B | 0.1106 | 1.56 | TP53 | 0.3748 | −1.64 | NFKB1 | 0.4643 | 2.20 |
| RTC | 0.9771 | 1.03 | SOD2 | 0.5508 | 1.27 | PPC | 0.8868 | −1.04 |

36y.o. idebenone + UVB Continuous Incubation

| | | | | | | | | |
|---|---|---|---|---|---|---|---|---|
| PARP1 | 0.6958 | 1.65 | COX1 | 0.6581 | 1.05 | KRAS | 0.0815 | 1.90 |
| IGF1 | 0.2636 | 3.42 | HPRT1 | 0.6295 | 1.83 | SOD1 | 0.6662 | −1.71 |
| SHC1 | 0.6305 | −1.49 | APOE | 0.6536 | 1.49 | HSPA1L | 0.4114 | −5.37 |
| IL1A | 0.6711 | 1.97 | TERT | 0.0182 | 4.36 | RTC | 0.7672 | 1.54 |
| NADSYN1 | 0.6175 | 1.44 | CYP19A1 | 0.4845 | 5.56 | ACE | 0.1097 | 2.34 |
| GAPDH | 0.6482 | −1.83 | TNF | 0.9041 | −1.09 | TP53 | 0.7623 | 1.21 |
| PARP2 | 0.3134 | 3.42 | PTGS2 | 0.5861 | 1.83 | SOD2 | 0.7245 | 1.17 |
| IGF2 | 0.7192 | −1.09 | HIGX1A | 0.3837 | 1.71 | CASP9 | 0.1702 | 5.19 |
| IFI44 | 0.0456 | 2.98 | HLA-DRA | 0.4650 | 3.93 | CCL4L1 | 0.3237 | 10.02 |
| IL6 | 0.6437 | −1.83 | TEP1 | 0.5204 | 1.13 | PPC | 0.5602 | 1.25 |
| BAX | 0.0789 | 2.26 | BCL2 | 0.3170 | 7.09 | GH1 | 0.4994 | 2.18 |
| ACTB | 0.3821 | −3.93 | HSPA1A | 0.7895 | 1.34 | MAPK14 | 0.2624 | 1.65 |
| PPARG | 0.1235 | 3.19 | HSPA1B | 0.6666 | 1.71 | NOS2A | 0.6051 | 3.80 |
| CLK1 | 0.3737 | 1.71 | RTC | 0.0152 | 10.02 | CASP2 | 0.9421 | 1.09 |
| CREBBP | 0.4980 | −1.21 | DDC | 0.4823 | 4.84 | NFKB1 | 0.0673 | 2.34 |
| IL10 | 0.9335 | 1.13 | SIRT1 | 0.3576 | 1.77 | PPC | 0.2434 | 1.21 |

36y.o. vs. 50y.o. idebenone + UVB Continuous Incubation

| | | | | | | | | |
|---|---|---|---|---|---|---|---|---|
| PARP1 | 0.6308 | 2.05 | COX1 | 0.4358 | −2.32 | KRAS | 0.6709 | 1.18 |
| IGF1 | 0.9077 | −1.08 | HPRT1 | 0.4416 | 2.20 | SOD1 | 0.4993 | 2.44 |
| SHC1 | 0.4475 | 1.67 | APOE | 0.1536 | −2.48 | HSPA1L | 0.8415 | 1.61 |
| IL1A | 0.3660 | 2.20 | TERT | 0.0884 | 2.13 | RTC | 0.9615 | −1.08 |
| NADSYN1 | 0.3313 | 2.28 | CYP19A1 | 0.5457 | −2.48 | ACE | 0.4018 | 1.40 |
| GAPDH | 0.6295 | 2.13 | TNF | 0.6489 | 1.40 | TP53 | 0.9357 | −1.04 |
| PARP2 | 0.4154 | −1.48 | PTGS2 | 0.3192 | 2.44 | SOD2 | 0.0137 | −1.38 |
| IGF2 | 0.6202 | −1.16 | HIGX1A | 0.5994 | −2.16 | CASP9 | 0.4706 | −1.88 |
| IFI44 | 0.3629 | −1.43 | HLA-DRA | 0.4815 | 1.31 | CCL4L1 | 0.6094 | −2.09 |
| IL6 | 0.2960 | 3.97 | TEP1 | 0.1526 | 1.31 | PPC | 0.1922 | −1.29 |
| BAX | 0.5314 | −1.64 | BCL2 | 0.4000 | −1.53 | GH1 | 0.5619 | 1.40 |
| ACTB | 0.2956 | 4.88 | HSPA1A | 0.4575 | 2.28 | MAPK14 | 0.9287 | 1.03 |
| PPARG | 0.2629 | −1.76 | HSPA1B | 0.3464 | 2.62 | NOS2A | 0.8137 | 1.56 |
| CLK1 | 0.8626 | −1.04 | RTC | 0.1718 | −1.38 | CASP2 | 0.8407 | −1.38 |
| CREBBP | 0.7530 | 1.14 | DDC | 0.5867 | −1.58 | NFKB1 | 0.1974 | 1.56 |
| IL10 | 0.9210 | 1.14 | SIRT1 | 0.9366 | −1.04 | PPC | 0.0298 | −1.88 |

50y.o. idebenone + UVB Continuous Incubation

| | | | | | | | | |
|---|---|---|---|---|---|---|---|---|
| PARP1 | 0.7305 | 1.67 | PPARG | 0.0400 | 3.22 | HLA-DRA | 0.3525 | 2.44 |
| IGF1 | 0.2181 | 3.45 | CLK1 | 0.1919 | 1.85 | TEP1 | 0.0577 | 1.79 |
| SHC1 | 0.3160 | 1.92 | CREBBP | 0.6487 | 1.18 | BCL2 | 0.4960 | 3.83 |
| IL1A | 0.1491 | 3.57 | IL10 | 0.6391 | 2.05 | HSPA1A | 0.3222 | 1.50 |
| NADSYN1 | 0.3617 | 1.56 | COX1 | 0.7259 | −1.43 | HSPA1B | 0.1872 | 3.70 |
| GAPDH | 0.7148 | −1.38 | HPRT1 | 0.1228 | 5.23 | RTC | 0.4698 | 2.44 |
| PARP2 | 0.2740 | 2.53 | APOE | 0.2488 | 1.18 | DDC | 0.6836 | 1.85 |
| IGF2 | 0.5859 | −1.12 | TERT | 0.0052 | 8.50 | SIRT1 | 0.1610 | 1.98 |
| IFI44 | 0.2737 | 1.56 | CYP19A1 | 0.8592 | 1.31 | KRAS | 0.3867 | 2.13 |
| IL6 | 0.1144 | 3.01 | TNF | 0.5149 | 1.50 | SOD1 | 0.8208 | 1.10 |
| BAX | 0.9572 | −1.04 | PTGS2 | 0.2987 | 2.71 | HSPA1L | 0.6642 | −2.48 |
| ACTB | 0.7158 | 1.31 | HIGX1A | 0.8878 | 1.22 | RTC | 0.5462 | 15.86 |
| ACE | 0.3049 | 1.92 | CCL4L1 | 0.8066 | 1.40 | NOS2A | 0.5885 | 1.98 |
| TP53 | 0.2533 | 1.67 | PPC | 0.7945 | 1.06 | CASP2 | 0.9451 | 1.14 |
| SOD2 | 0.9478 | 1.03 | GH1 | 0.1296 | 3.01 | NFKB1 | 0.1437 | 3.70 |
| CASP9 | 0.3199 | 2.13 | MAPK14 | 0.2623 | 2.44 | PPC | 0.4326 | 86.67 |

36y.o. vs. 50y.o. Green tea + UVB 4 hr Incubation

| | | | | | | | | |
|---|---|---|---|---|---|---|---|---|
| PARP1 | 0.9455 | −1.03 | COX1 | 0.3319 | −1.36 | KRAS | 0.7272 | 1.15 |
| IGF1 | 0.9806 | 1.04 | HPRT1 | 0.0199 | 1.87 | SOD1 | 0.4057 | −1.62 |
| SHC1 | 0.9146 | −1.03 | APOE | 0.5037 | 1.28 | HSPA1L | 0.5569 | −1.19 |
| IL1A | 0.1925 | 4.30 | TERT | 0.4513 | 2.08 | RTC | 0.2581 | −3.35 |
| NADSYN1 | 0.4987 | −1.27 | CYP19A1 | 0.9091 | −1.19 | ACE | 0.2501 | −1.27 |
| GAPDH | 0.0616 | 2.15 | TNF | 0.7305 | 2.08 | TP53 | 0.7480 | −1.14 |
| PARP2 | 0.7071 | 1.47 | PTGS2 | 0.1010 | 2.84 | SOD2 | 0.4452 | −1.27 |
| IGF2 | 0.2976 | −1.19 | HIGX1A | 0.8787 | 1.37 | CASP9 | 0.3311 | −1.41 |
| IFI44 | 0.2374 | −1.80 | HLA-DRA | 0.8536 | 1.63 | CCL4L1 | 0.9638 | −1.07 |
| IL6 | 0.4115 | 1.57 | TEP1 | 0.2106 | −1.46 | PPC | 0.4138 | −1.19 |
| BAX | 0.6342 | −1.27 | BCL2 | 0.3493 | 1.28 | GH1 | 0.7038 | 1.87 |
| ACTB | 0.1555 | 2.74 | HSPA1A | 0.5387 | −1.14 | MAPK14 | 0.1224 | −1.46 |
| PPARG | 0.6867 | −1.23 | HSPA1B | 0.2512 | −2.37 | NOS2A | 0.9356 | 1.28 |
| CLK1 | 0.5864 | −1.23 | RTC | 0.1180 | −1.86 | CASP2 | 0.2011 | −1.51 |
| CREBBP | 0.1450 | 1.52 | DDC | 0.5737 | 3.04 | NFKB1 | 0.9821 | 1.00 |
| IL10 | 0.4542 | 1.94 | SIRT1 | 0.4749 | −1.23 | PPC | 0.0403 | −2.45 |

36y.o. idebenone + UVB vs. Green tea + UVB 4 hr Incubation

| | | | | | | | | |
|---|---|---|---|---|---|---|---|---|
| PARP1 | 0.2751 | −1.57 | COX1 | 0.0898 | −1.81 | KRAS | 0.5290 | −1.69 |
| IGF1 | 0.0290 | 8.85 | HPRT1 | 0.0281 | 1.41 | SOD1 | 0.0012 | −1.32 |
| SHC1 | 0.4356 | −1.11 | APOE | 0.0831 | 1.41 | HSPA1L | 0.0917 | 1.80 |
| IL1A | 0.0872 | 7.70 | TERT | 0.0026 | 7.97 | RTC | 0.7221 | −1.32 |
| NADSYN1 | 0.1509 | −1.63 | CYP19A1 | 0.1530 | 9.48 | ACE | 0.1256 | −1.11 |
| GAPDH | 0.0219 | −1.57 | TNF | 0.2172 | 7.44 | TP53 | 0.5046 | −1.32 |
| PARP2 | 0.0280 | 7.44 | PTGS2 | 0.0043 | 2.14 | SOD2 | 0.4913 | −1.24 |
| IGF2 | 0.1047 | −1.15 | HIGX1A | 0.1399 | 17.09 | CASP9 | 0.0941 | −1.94 |
| IFI44 | 0.3355 | −1.28 | HLA-DRA | 0.1364 | 18.96 | CCL4L1 | 0.1364 | 9.48 |
| IL6 | 0.8036 | −1.04 | TEP1 | 0.9880 | −1.00 | PPC | 0.1764 | −1.57 |
| BAX | 0.2445 | −2.23 | BCL2 | 0.8224 | −1.15 | GH1 | 0.0405 | 16.51 |
| ACTB | 0.3675 | −1.57 | HSPA1A | 0.2762 | −1.87 | MAPK14 | 0.2004 | −1.63 |
| PPARG | 0.1103 | 1.74 | HSPA1B | 0.3274 | −2.08 | NOS2A | 0.1774 | 34.18 |
| CLK1 | 0.8434 | −1.11 | RTC | 0.0619 | −1.75 | CASP2 | 0.2713 | −2.47 |
| CREBBP | 0.9773 | −1.00 | DDC | 0.0122 | 42.08 | NFKB1 | 0.8543 | −1.04 |
| IL10 | 0.0023 | 4.74 | SIRT1 | 0.5766 | −1.15 | PPC | 0.0713 | −1.52 |

50y.o. idebenone + UVB vs. Green tea + UVB 4 hr Incubation

| | | | | | | | | |
|---|---|---|---|---|---|---|---|---|
| PARP1 | 0.2847 | 1.52 | COX1 | 0.7489 | −1.19 | KRAS | 0.0908 | −1.11 |
| IGF1 | 0.3659 | 4.77 | HPRT1 | 0.8907 | 1.04 | SOD1 | 0.4744 | 1.52 |
| SHC1 | 0.7443 | 1.11 | APOE | 0.9605 | 1.04 | HSPA1L | 0.0753 | 1.87 |
| IL1A | 0.7996 | 1.28 | TERT | 0.2766 | 3.37 | RTC | 0.4539 | 2.15 |
| NADSYN1 | 0.7508 | −1.19 | CYP19A1 | 0.1097 | 11.33 | ACE | 0.2054 | 1.32 |
| GAPDH | 0.3414 | −2.92 | TNF | 0.3671 | 6.98 | TP53 | 0.4846 | −1.51 |
| PARP2 | 0.1904 | 5.87 | PTGS2 | 0.1609 | 2.22 | SOD2 | 0.3448 | −1.32 |
| IGF2 | 0.9928 | 1.00 | HIGX1A | 0.2918 | 8.30 | CASP9 | 0.7777 | 1.15 |
| IFI44 | 0.2318 | 2.15 | HLA-DRA | 0.3021 | 16.03 | CCL4L1 | 0.1236 | 10.95 |
| IL6 | 0.5085 | −9.83 | TEP1 | 0.1166 | 2.55 | PPC | 0.9899 | 1.00 |
| BAX | 0.7394 | 1.11 | BCL2 | 0.2106 | 1.19 | GH1 | 0.2504 | 8.30 |
| ACTB | 0.3044 | −3.03 | HSPA1A | 0.4079 | 1.32 | MAPK14 | 0.1391 | 1.32 |
| PPARG | 0.2755 | 2.74 | HSPA1B | 0.2031 | 2.64 | NOS2A | 0.3034 | 17.18 |
| CLK1 | 0.7055 | −1.11 | RTC | 0.5785 | −1.19 | CASP2 | 0.6259 | −1.23 |
| CREBBP | 0.7860 | 1.11 | DDC | 0.2499 | 14.95 | NFKB1 | 0.8461 | −1.03 |
| IL10 | 0.2786 | 4.29 | SIRT1 | 0.2254 | 1.63 | PPC | 0.7181 | −1.11 |

36y.o. Green tea + UVB 4 hr Incubation vs. Continuous Incubation

| | | | | | | | | |
|---|---|---|---|---|---|---|---|---|
| PARP1 | 0.2811 | −1.52 | COX1 | 0.0845 | −1.81 | KRAS | 0.4744 | −1.37 |
| IGF1 | 0.2841 | 1.74 | HPRT1 | 0.0189 | −1.75 | SOD1 | 0.0206 | −1.57 |
| SHC1 | 0.1708 | −1.28 | APOE | 0.0836 | 1.14 | HSPA1L | 0.2702 | −1.52 |
| IL1A | 0.3156 | −1.75 | TERT | 0.0836 | 1.14 | RTC | 0.1795 | 1.23 |
| NADSYN1 | 0.7694 | −1.08 | CYP19A1 | 0.3658 | −3.26 | ACE | 0.8963 | 1.03 |
| GAPDH | 0.0691 | −1.32 | TNF | 0.4689 | −2.08 | TP53 | 0.4297 | −1.32 |
| PARP2 | 0.1074 | −3.26 | PTGS2 | 0.0021 | −2.47 | SOD2 | 0.9902 | −1.00 |
| IGF2 | 0.0836 | 1.14 | HIGX1A | 0.4579 | −2.65 | CASP9 | 0.1756 | −1.63 |
| IFI44 | 0.3903 | −1.24 | HLA-DRA | 0.3878 | −3.75 | CCL4L1 | 0.5984 | −1.75 |
| IL6 | 0.1107 | −1.94 | TEP1 | 0.3621 | −1.15 | PPC | 0.5936 | 1.11 |
| BAX | 0.5378 | −1.37 | BCL2 | 0.2290 | −1.37 | GH1 | 0.4896 | −1.63 |
| ACTB | 0.4678 | −1.42 | HSPA1A | 0.0058 | −1.52 | MAPK14 | 0.8366 | −1.04 |
| PPARG | 0.1510 | −1.94 | HSPA1B | 0.6170 | −1.19 | NOS2A | 0.4441 | −5.12 |
| CLK1 | 0.8237 | 1.07 | RTC | 0.5998 | 1.11 | CASP2 | 0.1932 | −1.52 |
| CREBBP | 0.0887 | −1.37 | DDC | 0.5213 | −1.37 | NFKB1 | 0.4491 | −1.28 |
| IL10 | 0.0836 | 1.14 | SIRT1 | 0.3878 | −1.28 | PPC | 0.5203 | 1.14 |

50y.o. Green tea + UVB 4 hr Incubation vs. Continuous Incubation

| | | | | | | | | |
|---|---|---|---|---|---|---|---|---|
| PARP1 | 0.9422 | 1.03 | COX1 | 0.9180 | −1.04 | KRAS | 0.2080 | −1.69 |
| IGF1 | 0.9656 | 1.07 | HPRT1 | 0.1915 | −2.74 | SOD1 | 0.3500 | 1.93 |
| SHC1 | 0.1656 | −1.69 | APOE | 0.4517 | −1.32 | HSPA1L | 0.4342 | 1.46 |
| IL1A | 0.4398 | −3.49 | TERT | 0.5360 | −1.81 | RTC | 0.9570 | 1.07 |
| NADSYN1 | 0.9394 | 1.03 | CYP19A1 | 0.5301 | −2.23 | ACE | 0.4997 | 1.23 |
| GAPDH | 0.3681 | −2.56 | TNF | 0.7750 | −1.87 | TP53 | 0.5849 | −1.63 |
| PARP2 | 0.7938 | −1.42 | PTGS2 | 0.5212 | 1.36 | SOD2 | 0.4428 | −1.63 |
| IGF2 | 0.3696 | 1.14 | HIGX1A | 0.9357 | −1.19 | CASP9 | 0.3539 | −1.63 |
| IFI44 | 0.7768 | −1.15 | HLA-DRA | 0.5547 | −4.16 | CCL4L1 | 0.9979 | −1.00 |
| IL6 | 0.9948 | −1.00 | TEP1 | 0.4344 | 1.56 | PPC | 0.5009 | 1.14 |
| BAX | 0.5314 | −1.24 | BCL2 | 0.5294 | −1.19 | GH1 | 0.4739 | −3.15 |
| ACTB | 0.9505 | −1.04 | HSPA1A | 0.9868 | −1.00 | MAPK14 | 0.3491 | −1.57 |
| PPARG | 0.8659 | −1.11 | HSPA1B | 0.3104 | 2.21 | NOS2A | 0.8772 | −1.57 |
| CLK1 | 0.4655 | 1.27 | RTC | 0.7621 | −1.28 | CASP2 | 0.0594 | −1.52 |
| CREBBP | 0.6715 | −1.11 | DDC | 0.6157 | −2.74 | NFKB1 | 0.5868 | −2.08 |
| IL10 | 0.4343 | −2.01 | SIRT1 | 0.0759 | −1.47 | PPC | 0.7505 | 1.14 |

36y.o.Green tea + UVB 4 hr Incubation

| | | | | | | | | |
|---|---|---|---|---|---|---|---|---|
| PARP1 | 0.0295 | 6.73 | COX1 | 0.1190 | 1.62 | KRAS | 0.1681 | 2.14 |
| IGF1 | 0.4765 | −2.14 | HPRT1 | 0.3778 | 2.46 | SOD1 | 0.0440 | 1.87 |
| SHC1 | 0.6132 | 1.32 | APOE | 0.4422 | −1.87 | HSPA1L | 0.3532 | −2.55 |
| IL1A | 0.4688 | −3.03 | TERT | 0.3442 | 1.07 | RTC | 0.1131 | 4.14 |
| NADSYN1 | 0.0326 | 3.61 | CYP19A1 | 0.9021 | −1.37 | ACE | 0.0130 | 2.55 |
| GAPDH | 0.3114 | 1.52 | TNF | 0.1540 | −8.88 | TP53 | 0.8100 | 1.15 |
| PARP2 | 0.5195 | −2.00 | PTGS2 | 0.8181 | 1.23 | SOD2 | 0.6069 | 1.32 |
| IGF2 | 0.5892 | −1.07 | HIGX1A | 0.2033 | −8.88 | CASP9 | 0.0757 | 4.00 |
| IFI44 | 0.0470 | 3.36 | HLA-DRA | 0.5230 | −4.44 | CCL4L1 | 0.9876 | 1.04 |
| IL6 | 0.6829 | 1.32 | TEP1 | 0.2874 | 1.23 | PPC | 0.2732 | 1.68 |
| BAX | 0.1428 | 3.14 | BCL2 | 0.3999 | 4.76 | GH1 | 0.2653 | −4.92 |
| ACTB | 0.7718 | −1.27 | HSPA1A | 0.0940 | 3.14 | MAPK14 | 0.1301 | 2.38 |
| PPARG | 0.7349 | 1.15 | HSPA1B | 0.2033 | 4.14 | NOS2A | 0.7959 | −2.07 |
| CLK1 | 0.4533 | 1.57 | RTC | 0.0178 | 8.28 | CASP2 | 0.3052 | 3.25 |
| CREBBP | 0.2277 | −1.32 | DDC | 0.3650 | −8.88 | NFKB1 | 0.0083 | 3.36 |
| IL10 | 0.4329 | −3.25 | SIRT1 | 0.2003 | 1.62 | PPC | 0.0562 | 1.87 |

50y.o.Green tea + UVB 4 hr Incubation

| | | | | | | | | |
|---|---|---|---|---|---|---|---|---|
| PARP1 | 0.4175 | 3.20 | SHC1 | 0.2847 | 2.19 | NADSYN1 | 0.5072 | 1.35 |
| IGF1 | 0.6921 | −1.89 | IL1A | 0.8694 | 1.17 | GAPDH | 0.0711 | 2.04 |
| PARP2 | 0.8505 | −1.25 | CYP19A1 | 0.4086 | −2.77 | HSPA1L | 0.4952 | −2.25 |
| IGF2 | 0.4113 | −1.13 | TNF | 0.5152 | −3.66 | RTC | 0.5641 | 13.74 |
| IFI44 | 0.4078 | 1.39 | PTGS2 | 0.3757 | 2.11 | ACE | 0.7704 | 1.17 |
| IL6 | 0.2000 | 2.89 | HIGX1A | 0.4349 | −4.20 | TP53 | 0.2702 | 1.44 |
| BAX | 0.2051 | 1.72 | HLA-DRA | 0.4958 | −5.74 | SOD2 | 0.5834 | 1.26 |
| ACTB | 0.2854 | 2.27 | TEP1 | 0.9368 | 1.02 | CASP9 | 0.3102 | 2.19 |
| PPARG | 0.3688 | 1.66 | BCL2 | 0.4192 | 5.03 | CCL4L1 | 0.3063 | −3.53 |
| CLK1 | 0.4276 | 1.44 | HSPA1A | 0.4680 | 1.35 | PPC | 0.1531 | 1.55 |
| CREBBP | 0.1528 | 1.44 | HSPA1B | 0.7003 | 1.44 | GH1 | 0.5420 | −2.68 |
| IL10 | 0.9672 | −1.05 | RTC | 0.7328 | 1.49 | MAPK14 | 0.2449 | 2.35 |
| COX1 | 0.1221 | 1.84 | DDC | 0.5064 | −4.82 | NOS2A | 0.5324 | −4.82 |
| HPRT1 | 0.0688 | 5.98 | SIRT1 | 0.2780 | 1.55 | CASP2 | 0.4170 | 3.10 |
| APOE | 0.4271 | 1.35 | KRAS | 0.3073 | 2.35 | NFKB1 | 0.1601 | 3.43 |
| TERT | 0.4244 | 2.19 | SOD1 | 0.8474 | −1.13 | PPC | 0.4185 | 102.54 |

36y.o. vs. 50y.o.Green tea + UVB Continuous Incubation

| | | | | | | | | |
|---|---|---|---|---|---|---|---|---|
| PARP1 | 0.2385 | 1.52 | COX1 | 0.4812 | 1.28 | KRAS | 0.8432 | −1.07 |
| IGF1 | 0.1451 | −1.56 | HPRT1 | 0.7651 | 1.19 | SOD1 | 0.1645 | 1.87 |
| SHC1 | 0.0686 | −1.36 | APOE | 0.0260 | −1.19 | HSPA1L | 0.2672 | 1.87 |
| IL1A | 0.5737 | 2.15 | TERT | 0.9832 | 1.00 | RTC | 0.2189 | −3.85 |
| NADSYN1 | 0.7024 | −1.14 | CYP19A1 | 0.7801 | 1.24 | ACE | 0.8424 | −1.07 |
| GAPDH | 0.9047 | 1.11 | TNF | 0.4828 | 2.31 | TP53 | 0.6813 | −1.41 |
| PARP2 | 0.2997 | 3.38 | PTGS2 | 0.0063 | 9.55 | SOD2 | 0.2934 | −2.06 |
| IGF2 | 0.0260 | −1.19 | HIGX1A | 0.4688 | 3.04 | CASP9 | 0.4694 | −1.41 |
| IFI44 | 0.2551 | −1.68 | HLA-DRA | 0.3510 | 1.47 | CCL4L1 | 0.5776 | 1.63 |
| IL6 | 0.0622 | 3.04 | TEP1 | 0.6710 | 1.24 | PPC | 0.4866 | −1.14 |
| BAX | 0.6751 | −1.14 | BCL2 | 0.2200 | 1.47 | GH1 | 0.8050 | −1.03 |
| ACTB | 0.1171 | 3.75 | HSPA1A | 0.0214 | 1.32 | MAPK14 | 0.1729 | −2.21 |
| PPARG | 0.5229 | 1.42 | HSPA1B | 0.8052 | 1.11 | NOS2A | 0.4592 | 4.16 |
| CLK1 | 0.8872 | −1.03 | RTC | 0.2962 | −2.63 | CASP2 | 0.0560 | −1.51 |
| CREBBP | 0.0592 | 1.87 | DDC | 0.5330 | 1.52 | NFKB1 | 0.7186 | −1.62 |
| IL10 | 0.0260 | −1.19 | SIRT1 | 0.0633 | −1.41 | PPC | 0.1334 | −2.45 |

50y.o. idebenone + UVB vs. Green tea + UVB Continuous Incubation

| | | | | | | | | |
|---|---|---|---|---|---|---|---|---|
| PARP1 | 0.4327 | −1.98 | COX1 | 0.4156 | −2.53 | KRAS | 0.3981 | 1.53 |
| IGF1 | 0.0984 | 6.12 | HPRT1 | 0.3156 | 2.40 | SOD1 | 0.3574 | −1.56 |
| SHC1 | 0.0439 | 1.48 | APOE | 0.2989 | 1.16 | HSPA1L | 0.7893 | −1.61 |
| IL1A | 0.1725 | 10.65 | TERT | 0.0097 | 7.53 | RTC | 0.9509 | 1.08 |
| NADSYN1 | 0.7886 | 1.12 | CYP19A1 | 0.2735 | 8.07 | ACE | 0.3638 | 1.33 |
| GAPDH | 0.9387 | −1.10 | TNF | 0.1697 | 10.29 | TP53 | 0.5055 | 1.88 |
| PARP2 | 0.2127 | 4.48 | PTGS2 | 0.9117 | −1.06 | SOD2 | 0.6341 | 1.33 |
| IGF2 | 0.5396 | −1.14 | HIGX1A | 0.4135 | 6.12 | CASP9 | 0.3684 | 1.58 |
| IFI44 | 0.6115 | 1.29 | HLA-DRA | 0.0106 | 58.18 | CCL4L1 | 0.3753 | 4.97 |
| IL6 | 0.8863 | 1.04 | TEP1 | 0.8154 | 1.12 | PPC | 0.1028 | −1.67 |
| BAX | 0.6522 | −1.45 | BCL2 | 0.8242 | −1.10 | GH1 | 0.0083 | 25.33 |
| ACTB | 0.4972 | −1.67 | HSPA1A | 0.5047 | 1.12 | MAPK14 | 0.3843 | 1.64 |
| PPARG | 0.2108 | 2.16 | HSPA1B | 0.6913 | 1.16 | NOS2A | 0.2822 | 15.06 |
| CLK1 | 0.9666 | 1.01 | RTC | 0.3960 | 2.09 | CASP2 | 0.6879 | −1.79 |
| CREBBP | 0.8070 | −1.10 | DDC | 0.0696 | 24.46 | NFKB1 | 0.5519 | 2.24 |
| IL10 | 0.3056 | 4.32 | SIRT1 | 0.0542 | 1.88 | PPC | 0.4826 | −1.35 |

36y.o. IDE + UVB vs. Green tea + UVB Continuous Incubation

| | | | | | | | | |
|---|---|---|---|---|---|---|---|---|
| PARP1 | 0.4651 | −2.68 | IL1A | 0.0796 | 10.41 | IGF2 | 0.4938 | −1.16 |
| IGF1 | 0.0062 | 4.23 | NADSYN1 | 0.3024 | −2.33 | IFI44 | 0.6033 | 1.09 |
| SHC1 | 0.5133 | −1.54 | GAPDH | 0.5700 | −2.10 | IL6 | 0.8434 | −1.25 |
| ACTB | 0.5422 | −2.17 | PARP2 | 0.0227 | 22.32 | BAX | 0.8781 | −1.01 |
| PPARG | 0.0524 | 5.39 | HLA-DRA | 0.0013 | 65.34 | TP53 | 0.4512 | 1.39 |
| CLK1 | 0.9362 | 1.02 | TEP1 | 0.7221 | 1.06 | SOD2 | 0.0085 | −1.13 |
| CREBBP | 0.1755 | 1.49 | BCL2 | 0.1080 | 2.04 | CASP9 | 0.4027 | 2.11 |
| IL10 | 0.1181 | 3.20 | HSPA1A | 0.6773 | −1.54 | CCL4L1 | 0.0061 | 16.91 |
| COX1 | 0.2553 | 1.17 | HSPA1B | 0.4770 | −2.03 | PPC | 0.0622 | −1.48 |
| HPRT1 | 0.7497 | 1.30 | RTC | 0.6414 | 1.09 | GH1 | 0.0195 | 17.51 |
| APOE | 0.1517 | 2.43 | DDC | 0.0020 | 58.89 | MAPK14 | 0.0945 | −1.39 |
| TERT | 0.0234 | 3.56 | SIRT1 | 0.5490 | 1.39 | NOS2A | 0.1056 | 40.22 |
| CYP19A1 | 0.0078 | 24.76 | KRAS | 0.4295 | 1.21 | CASP2 | 0.4090 | −1.96 |
| TNF | 0.0168 | 16.91 | SOD1 | 0.5754 | −2.03 | NFKB1 | 0.7575 | −1.13 |
| PTGS2 | 0.1249 | 3.68 | HSPA1L | 0.8442 | −1.39 | PPC | 0.0575 | −1.77 |
| HIGX1A | 0.0003 | 40.22 | RTC | 0.4162 | −3.29 | | | |
| | | | ACE | 0.7727 | −1.13 | | | |

DATA TABLE 5 (in 4 parts)
Gene expression profile related to aging, lifespan and telomerase length maintenance
of cells contacted with green tea polyphenols and coffee cherry extract
36 y.o. Coffee cherry + UVB vs. Green tea + UVB 4 hr Incubation

| | | | | | | | | |
|---|---|---|---|---|---|---|---|---|
| PARP1 | 0.2232 | −3.06 | COX1 | 0.3442 | −3.17 | KRAS | 0.3068 | −1.82 |
| IGF1 | 0.0374 | 8.20 | HPRT1 | 0.5226 | −2.32 | SOD1 | 0.3347 | −3.64 |
| SHC1 | 0.6156 | −1.12 | APOE | 0.5709 | 1.22 | HSPA1L | 0.1778 | 1.50 |
| IL1A | 0.1836 | 3.96 | TERT | 0.0038 | 7.65 | RTC | 0.1734 | −1.59 |
| NADSYN1 | 0.2835 | −1.38 | CYP19A1 | 0.1394 | 12.00 | ACE | 0.3830 | −1.82 |
| GAPDH | 0.3373 | −3.17 | TNF | 0.3836 | 3.82 | TP53 | 0.6925 | 1.22 |
| PARP2 | 0.0541 | 6.66 | PTGS2 | 0.8225 | −1.29 | SOD2 | 0.6046 | 1.31 |
| IGF2 | 0.9535 | −1.01 | HIGX1A | 0.2287 | 7.39 | CASP9 | 0.1084 | −1.82 |
| IFI44 | 0.6022 | 1.66 | HLA-DRA | 0.1316 | 23.18 | CCL4L1 | 0.2460 | 5.80 |
| IL6 | 0.5708 | 1.06 | TEP1 | 0.3379 | −1.48 | PPC | 0.7687 | −1.08 |
| BAX | 0.3068 | −2.02 | BCL2 | 0.3725 | 2.05 | GH1 | 0.0925 | 8.20 |
| ACTB | 0.7175 | 1.18 | HSPA1A | 0.2495 | −3.52 | MAPK14 | 0.0502 | −1.95 |
| PPARG | 0.7607 | 1.18 | HSPA1B | 0.2568 | −3.40 | NOS2A | 0.2281 | 19.49 |
| CLK1 | 0.9808 | −1.01 | RTC | 0.1264 | −2.32 | CASP2 | 0.3152 | −3.52 |
| CREBBP | 0.7705 | 1.06 | DDC | 0.0231 | 46.37 | NFKB1 | 0.2760 | −3.77 |
| IL10 | 0.1417 | 5.80 | SIRT1 | 0.7107 | 1.26 | PPC | 0.2867 | −1.43 |

50 y.o. Coffee cherry + UVB vs. Green tea + UVB 4 hr Incubation

| | | | | | | | | |
|---|---|---|---|---|---|---|---|---|
| PARP1 | 0.6579 | −1.26 | COX1 | 0.4140 | −1.35 | KRAS | 0.3418 | −1.92 |
| IGF1 | 0.2950 | 7.03 | HPRT1 | 0.9281 | 1.01 | SOD1 | 0.4140 | 1.64 |
| SHC1 | 0.3292 | −1.85 | APOE | 0.9852 | 1.01 | HSPA1L | 0.7855 | 1.33 |
| IL1A | 0.3177 | 2.76 | TERT | 0.2278 | 4.63 | RTC | 0.4663 | 2.16 |
| NADSYN1 | 0.8352 | −1.10 | CYP19A1 | 0.1993 | 8.07 | ACE | 0.8404 | 1.08 |
| GAPDH | 0.2658 | −1.50 | TNF | 0.3308 | 8.35 | TP53 | 0.7795 | 1.08 |
| PARP2 | 0.2280 | 4.80 | PTGS2 | 0.5213 | 1.38 | SOD2 | 0.1795 | −1.67 |
| IGF2 | 0.6492 | −1.06 | HIGX1A | 0.2416 | 11.81 | CASP9 | 0.8143 | −1.10 |
| IFI44 | 0.9697 | −1.03 | HLA-DRA | 0.3316 | 13.11 | CCL4L1 | 0.1523 | 10.29 |
| IL6 | 0.8047 | 1.16 | TEP1 | 0.0710 | 1.95 | PPC | 0.4378 | −1.14 |
| BAX | 0.5831 | −1.26 | BCL2 | 0.7190 | −1.18 | GH1 | 0.2462 | 8.65 |
| ACTB | 0.1751 | −3.01 | HSPA1A | 0.3597 | 1.24 | MAPK14 | 0.5734 | 1.04 |
| PPARG | 0.2723 | 2.32 | HSPA1B | 0.3979 | 1.70 | NOS2A | 0.4123 | 10.65 |
| CLK1 | 0.6470 | 1.12 | RTC | 0.8377 | −1.22 | CASP2 | 0.0412 | 1.24 |
| CREBBP | 0.9881 | 1.01 | DDC | 0.2963 | 10.29 | NFKB1 | 0.4463 | −2.28 |
| IL10 | 0.2992 | 3.76 | SIRT1 | 0.0823 | 1.38 | PPC | 0.8125 | −1.06 |

36 y.o. Coffee cherry + UVB vs. Green tea + UVB Continuous Incubation

| | | | | | | | | |
|---|---|---|---|---|---|---|---|---|
| PARP1 | 0.1092 | 1.11 | COX1 | 0.1739 | 1.19 | KRAS | 0.4204 | 1.37 |
| IGF1 | 0.1004 | 3.15 | HPRT1 | 0.0529 | 2.23 | SOD1 | 0.0340 | 1.42 |
| SHC1 | 0.1798 | 1.24 | APOE | 0.7266 | 1.15 | HSPA1L | 0.0517 | 2.74 |
| IL1A | 0.0364 | 13.04 | TERT | 0.1089 | 2.94 | RTC | 0.6065 | −1.07 |
| NADSYN1 | 0.3160 | −1.14 | CYP19A1 | 0.0205 | 20.46 | ACE | 0.7317 | −1.14 |
| GAPDH | 0.1168 | 1.32 | TNF | 0.0094 | 18.44 | TP53 | 0.4565 | 1.32 |
| PARP2 | 0.0276 | 12.60 | PTGS2 | 0.0246 | 4.77 | SOD2 | 0.7520 | 1.08 |
| IGF2 | 0.0081 | −1.36 | HIGX1A | 0.0016 | 36.89 | CASP9 | 0.3903 | 1.24 |
| IFI44 | 0.1455 | 1.42 | HLA-DRA | 0.0073 | 40.93 | CCL4L1 | 0.0070 | 16.62 |
| IL6 | 0.2451 | 1.69 | TEP1 | 0.6140 | 1.11 | PPC | 0.0604 | −1.86 |
| BAX | 0.6092 | 1.11 | BCL2 | 0.0496 | 1.81 | GH1 | 0.0115 | 19.09 |
| ACTB | 0.0873 | 1.28 | HSPA1A | 0.3747 | 1.28 | MAPK14 | 0.9423 | −1.03 |
| PPARG | 0.0491 | 3.38 | HSPA1B | 0.6644 | 1.11 | NOS2A | 0.0117 | 90.82 |
| CLK1 | 0.2939 | −1.14 | RTC | 0.7275 | −1.03 | CASP2 | 0.5105 | 1.11 |
| CREBBP | 0.1149 | 1.37 | DDC | 0.0121 | 33.24 | NFKB1 | 0.8932 | 1.04 |
| IL10 | 0.1522 | 3.04 | SIRT1 | 0.6603 | 1.15 | PPC | 0.0471 | −1.99 |

50 y.o. Coffee cherry + UVB vs. Green tea + UVB Continuous Incubation

| | | | | | | | | |
|---|---|---|---|---|---|---|---|---|
| PARP1 | 0.5739 | 1.18 | COX1 | 0.9022 | −1.04 | KRAS | 0.3084 | 1.56 |
| IGF1 | 0.0860 | 4.11 | HPRT1 | 0.2571 | 2.28 | SOD1 | 0.5418 | −1.33 |
| SHC1 | 0.0513 | 1.51 | APOE | 0.3324 | 1.18 | HSPA1L | 0.3904 | 1.51 |
| IL1A | 0.1819 | 9.45 | TERT | 0.0147 | 7.41 | RTC | 0.2517 | 3.34 |
| NADSYN1 | 0.3584 | 1.51 | CYP19A1 | 0.0666 | 18.25 | ACE | 0.5063 | 1.36 |
| GAPDH | 0.5193 | 1.85 | TNF | 0.1103 | 15.35 | TP53 | 0.4053 | 2.13 |
| PARP2 | 0.1536 | 6.23 | PTGS2 | 0.8229 | 1.10 | SOD2 | 0.4068 | 1.73 |
| IGF2 | 0.0138 | −1.28 | HIGX1A | 0.1622 | 16.45 | CASP9 | 0.6640 | 1.36 |
| IFI44 | 0.5014 | 1.31 | HLA-DRA | 0.0099 | 51.63 | CCL4L1 | 0.1145 | 8.22 |
| IL6 | 0.5114 | 1.18 | TEP1 | 0.3978 | 1.61 | PPC | 0.2432 | −1.28 |
| BAX | 0.2774 | 1.51 | BCL2 | 0.3930 | 1.31 | GH1 | 0.0625 | 15.89 |
| ACTB | 0.6703 | −1.28 | HSPA1A | 0.4369 | −62.25 | MAPK14 | 0.3043 | 1.67 |
| PPARG | 0.1872 | 2.36 | HSPA1B | 0.7306 | −1.16 | NOS2A | 0.1746 | 26.72 |
| CLK1 | 0.3554 | −1.38 | RTC | 0.3249 | 2.45 | CASP2 | 0.0769 | 1.40 |
| CREBBP | 0.2221 | 1.40 | DDC | 0.0612 | 17.03 | NFKB1 | 0.7202 | 1.61 |
| IL10 | 0.0067 | 7.41 | SIRT1 | 0.0175 | 2.13 | PPC | 0.3724 | −1.47 |

DATA TABLE 6 (in 6 parts)
UVA/UVB injury to cells, which demonstrates a decrease in lifespan
or telomere length maintenance gene expression profiles
36 y.o. UVA1

| | | | | | | | | |
|---|---|---|---|---|---|---|---|---|
| PARP1 | 0.6019 | −1.69 | COX1 | 0.4399 | −306.02 | KRAS | 0.9327 | 1.10 |
| IGF1 | 0.9918 | 1.03 | HPRT1 | 0.4263 | 31.83 | SOD1 | 0.1275 | −14.50 |
| SHC1 | 0.4908 | −9.56 | APOE | 0.4569 | 18.93 | HSPA1L | 0.5133 | 9.46 |
| IL1A | 0.7137 | 3.35 | TERT | 0.0311 | −3.62 | RTC | 0.4045 | −4.46 |
| NADSYN1 | 0.2027 | −7.50 | CYP19A1 | 0.0637 | −15.01 | ACE | 0.3698 | 4.90 |
| GAPDH | 0.4934 | −7.50 | TNF | 0.8109 | −1.75 | TP53 | 0.4772 | −2.65 |
| PARP2 | 0.5623 | 45.02 | PTGS2 | 0.0576 | −2.48 | SOD2 | 0.6186 | −2.48 |
| IGF2 | 0.4005 | 8.83 | HIGX1A | 0.5860 | −3.75 | CASP9 | 0.5657 | 3.02 |
| IFI44 | 0.4251 | −21.22 | HLA-DRA | 0.4868 | −3.50 | CCL4L1 | 0.8610 | −1.69 |
| IL6 | 0.3932 | 2.81 | TEP1 | 0.3944 | −1.81 | PPC | 0.4771 | −11.77 |
| BAX | 0.4372 | −6.76 | BCL2 | 0.7568 | 1.41 | GH1 | 0.7481 | 5.63 |
| ACTB | 0.7563 | −1.81 | HSPA1A | 0.3878 | −10.61 | MAPK14 | 0.6730 | −2.15 |
| PPARG | 0.4546 | 49.95 | HSPA1B | 0.2932 | 1.56 | NOS2A | 0.4409 | −9.90 |
| CLK1 | 0.5876 | −4.31 | RTC | 0.8214 | −1.28 | CASP2 | 0.1041 | −2.23 |
| CREBBP | 0.4187 | 4.26 | DDC | 0.9804 | 1.10 | NFKB1 | 0.2173 | −1.47 |
| IL10 | 0.6172 | 4.12 | SIRT1 | 0.1340 | −3.75 | PPC | 0.5841 | −3.27 |

36 y.o. vs. 50 y.o. UVA1

| | | | | | | | | |
|---|---|---|---|---|---|---|---|---|
| PARP1 | 0.3134 | 49.69 | COX1 | 0.9979 | 1.02 | KRAS | 0.6542 | 1.66 |
| IGF1 | 0.9762 | −1.12 | HPRT1 | 0.9659 | 1.26 | SOD1 | 0.8060 | 1.91 |
| SHC1 | 0.9352 | −1.43 | APOE | 0.7272 | −4.33 | HSPA1L | 0.9588 | 1.26 |
| IL1A | 0.9174 | −1.53 | TERT | 0.2607 | −1.29 | RTC | 0.9292 | 1.26 |
| NADSYN1 | 0.8029 | 1.72 | CYP19A1 | 0.5800 | 1.61 | ACE | 0.6342 | 4.55 |
| GAPDH | 0.9954 | 1.02 | TNF | 0.9974 | −1.01 | TP53 | 0.9272 | −1.24 |
| PARP2 | 0.8920 | −2.96 | PTGS2 | 0.2442 | 3.11 | SOD2 | 0.8441 | −1.64 |
| IGF2 | 0.9975 | −1.01 | HIGX1A | 0.8985 | 1.61 | CASP9 | 0.9016 | −1.29 |
| IFI44 | 0.9384 | −1.43 | HLA-DRA | 0.7740 | 3.82 | CCL4L1 | 0.9979 | −1.01 |
| IL6 | 0.9210 | −1.12 | TEP1 | 0.6392 | 1.45 | PPC | 0.9490 | −1.33 |
| BAX | 0.9046 | 1.45 | BCL2 | 0.8454 | 1.26 | GH1 | 0.9795 | 1.22 |
| ACTB | 0.8469 | −1.76 | HSPA1A | 0.9978 | −1.01 | MAPK14 | 0.9312 | −1.20 |
| PPARG | 0.5351 | −31.23 | HSPA1B | 0.6026 | −1.16 | NOS2A | 0.8973 | −1.59 |
| CLK1 | 0.9496 | −1.24 | RTC | 0.9760 | −1.05 | CASP2 | 0.3468 | 1.61 |
| CREBBP | 0.9112 | −1.24 | DDC | 0.9595 | 1.35 | NFKB1 | 0.8640 | −1.20 |
| IL10 | 0.9400 | 1.35 | SIRT1 | 0.7123 | 1.72 | PPC | 0.9229 | −1.29 |

50 y.o. UVA1

| | | | | | | | | |
|---|---|---|---|---|---|---|---|---|
| PARP1 | 0.3299 | 39.53 | COX1 | 0.4333 | −174.25 | KRAS | 0.4081 | 2.39 |
| IGF1 | 0.8095 | −1.86 | HPRT1 | 0.3538 | 57.88 | SOD1 | 0.4779 | −6.25 |
| SHC1 | 0.5825 | −6.25 | APOE | 0.4152 | 6.08 | HSPA1L | 0.5190 | 8.91 |
| IL1A | 0.8199 | 1.81 | TERT | 0.0311 | −7.19 | RTC | 0.6763 | −2.54 |
| NADSYN1 | 0.4787 | −3.99 | CYP19A1 | 0.0193 | −10.52 | ACE | 0.2771 | 34.42 |
| GAPDH | 0.4522 | −13.88 | TNF | 0.6027 | −3.35 | TP53 | 0.6369 | −3.02 |
| PARP2 | 0.6896 | 7.75 | PTGS2 | 0.6061 | −1.74 | SOD2 | 0.7614 | −1.80 |
| IGF2 | 0.3983 | 9.22 | HIGX1A | 0.7364 | −2.72 | CASP9 | 0.3463 | 3.49 |
| IFI44 | 0.4634 | −11.67 | HLA-DRA | 0.8798 | −1.93 | CCL4L1 | 0.8018 | −1.99 |
| IL6 | 0.7267 | 1.42 | TEP1 | 0.9960 | 1.00 | PPC | 0.4424 | −14.37 |
| BAX | 0.5577 | −3.99 | BCL2 | 0.5252 | 1.57 | GH1 | 0.7493 | 6.08 |
| ACTB | 0.5232 | −4.91 | HSPA1A | 0.5610 | −6.04 | MAPK14 | 0.9024 | −1.19 |
| PPARG | 0.7570 | 2.01 | HSPA1B | 0.1665 | 2.31 | NOS2A | 0.3043 | −17.69 |
| CLK1 | 0.5678 | −4.13 | RTC | 0.9345 | −1.11 | CASP2 | 0.8126 | 1.19 |
| CREBBP | 0.3608 | 3.38 | DDC | 0.9427 | 1.37 | NFKB1 | 0.5969 | −1.86 |
| IL10 | 0.7419 | 2.84 | SIRT1 | 0.7638 | −1.56 | PPC | 0.5187 | −3.02 |

36 y.o. vs. 50 y.o. UVB

| | | | | | | | | |
|---|---|---|---|---|---|---|---|---|
| PARP1 | 0.5927 | 2.03 | COX1 | 0.1305 | −1.54 | KRAS | 0.9418 | 1.05 |
| IGF1 | 0.9305 | −1.09 | HPRT1 | 0.8077 | −1.30 | SOD1 | 0.4919 | 1.30 |
| SHC1 | 0.5051 | −1.71 | APOE | 0.4106 | −1.97 | HSPA1L | 0.8329 | −1.34 |
| IL1A | 0.8980 | 1.21 | TERT | 0.6951 | 1.02 | RTC | 0.5895 | −11.12 |
| NADSYN1 | 0.1532 | 2.11 | CYP19A1 | 0.8190 | 1.71 | ACE | 0.3686 | 1.71 |
| GAPDH | 0.2712 | 1.60 | TNF | 0.7915 | −1.17 | TP53 | 0.4826 | −1.44 |
| PARP2 | 0.9414 | −1.09 | PTGS2 | 0.6561 | 1.65 | SOD2 | 0.7491 | −1.21 |
| IGF2 | 0.3511 | −1.13 | HIGX1A | 0.4669 | −1.54 | CASP9 | 0.7298 | 1.30 |
| IFI44 | 0.3303 | 1.34 | HLA-DRA | 0.6992 | 2.11 | CCL4L1 | 0.5578 | 3.42 |
| IL6 | 0.6771 | −1.39 | TEP1 | 0.3966 | −1.21 | PPC | 0.8329 | −1.09 |
| BAX | 0.4278 | 1.44 | BCL2 | 0.9378 | 1.21 | GH1 | 0.9868 | 1.02 |
| ACTB | 0.9545 | −1.05 | HSPA1A | 0.2702 | 2.03 | MAPK14 | 0.6119 | −1.44 |
| PPARG | 0.2515 | −1.77 | HSPA1B | 0.8646 | 1.21 | NOS2A | 0.6066 | 2.98 |
| CLK1 | 0.8387 | −1.13 | RTC | 0.4033 | 2.98 | CASP2 | 0.8181 | −1.44 |
| CREBBP | 0.2539 | −1.25 | DDC | 0.8363 | 1.65 | NFKB1 | 0.9780 | −1.02 |
| IL10 | 0.7743 | −1.60 | SIRT1 | 0.6692 | −1.17 | PPC | 0.3963 | −134.83 |

36 y.o. UVB

| | | | | | | | | |
|---|---|---|---|---|---|---|---|---|
| PARP1 | 0.0390 | −5.14 | IGF1 | 0.5358 | −1.82 | SHC1 | 0.7004 | −1.24 |
| IL1A | 0.5073 | −2.66 | APOE | 0.6881 | 1.40 | SOD1 | 0.2453 | −1.58 |
| NADSYN1 | 0.0320 | −3.06 | TERT | 0.0139 | −4.63 | HSPA1L | 0.4439 | 2.05 |
| GAPDH | 0.7922 | −1.12 | CYP19A1 | 0.5255 | −4.63 | RTC | 0.1953 | −2.95 |
| PARP2 | 0.7852 | −1.33 | TNF | 0.5141 | 1.50 | ACE | 0.1440 | −1.88 |
| IGF2 | 0.3014 | 1.18 | PTGS2 | 0.5426 | −1.82 | TP53 | 0.6960 | 1.35 |
| IFI44 | 0.0279 | −3.39 | HIGX1A | 0.8374 | −1.12 | SOD2 | 0.6423 | −1.24 |
| IL6 | 0.9390 | 1.06 | HLA-DRA | 0.6655 | −2.16 | CASP9 | 0.1531 | −2.48 |
| BAX | 0.1614 | −2.24 | TEP1 | 0.4029 | −1.29 | CCL4L1 | 0.3710 | −7.53 |
| ACTB | 0.4206 | 2.13 | BCL2 | 0.3978 | −4.80 | PPC | 0.9847 | −1.01 |
| PPARG | 0.2929 | −1.88 | HSPA1A | 0.1485 | −2.95 | GH1 | 0.4738 | −2.16 |
| CLK1 | 0.7970 | 1.14 | HSPA1B | 0.2695 | −3.17 | MAPK14 | 0.2555 | −1.88 |
| CREBBP | 0.4552 | 1.18 | RTC | 0.0492 | −5.71 | NOS2A | 0.3127 | −10.65 |
| IL10 | 0.8795 | 1.26 | DDC | 0.6977 | −2.32 | CASP2 | 0.4019 | −2.48 |
| COX1 | 0.1618 | −1.20 | SIRT1 | 0.8669 | −1.04 | NFKB1 | 0.0281 | −2.32 |
| HPRT1 | 0.3931 | −2.57 | KRAS | 0.4241 | −1.53 | PPC | 0.4855 | 1.14 |

50 y.o. UVB

| | | | | | | | | |
|---|---|---|---|---|---|---|---|---|
| PARP1 | 0.6703 | −1.88 | COX1 | 0.8225 | −1.08 | KRAS | 0.9231 | −1.12 |
| IGF1 | 0.0942 | −3.39 | HPRT1 | 0.4021 | −2.31 | SOD1 | 0.9903 | −1.01 |
| SHC1 | 0.9663 | 1.03 | APOE | 0.2929 | −1.01 | HSPA1L | 0.9144 | 1.14 |
| IL1A | 0.2684 | −2.66 | TERT | 0.0308 | −7.01 | RTC | 0.4923 | −23.59 |
| NADSYN1 | 0.5142 | −1.33 | CYP19A1 | 0.1833 | −3.05 | ACE | 0.6257 | 1.40 |
| GAPDH | 0.1302 | −1.33 | TNF | 0.3845 | −1.47 | TP53 | 0.9733 | 1.03 |
| PARP2 | 0.3142 | −2.85 | PTGS2 | 0.3956 | −2.39 | SOD2 | 0.5980 | 1.51 |
| IGF2 | 0.4364 | 1.10 | HIGX1A | 0.1883 | −2.01 | CASP9 | 0.7882 | −1.28 |
| IFI44 | 0.9548 | 1.03 | HLA-DRA | 0.4101 | −2.16 | CCL4L1 | 0.0797 | −2.57 |
| IL6 | 0.3946 | −2.31 | TEP1 | 0.6754 | −1.24 | PPC | 0.9759 | −1.01 |
| BAX | 0.6937 | −1.33 | BCL2 | 0.4584 | −4.47 | GH1 | 0.1745 | −2.39 |
| ACTB | 0.7484 | 1.31 | HSPA1A | 0.7975 | 1.22 | MAPK14 | 0.8113 | −1.24 |
| PPARG | 0.2538 | −2.66 | HSPA1B | 0.6362 | −1.53 | NOS2A | 0.1423 | −4.03 |
| CLK1 | 0.6732 | 1.31 | RTC | 0.7227 | −1.58 | CASP2 | 0.5995 | −2.16 |
| CREBBP | 0.8478 | −1.08 | DDC | 0.7409 | −1.53 | NFKB1 | 0.3020 | −2.48 |
| IL10 | 0.4711 | −2.48 | SIRT1 | 0.8525 | 1.14 | PPC | 0.4343 | −85.04 |

DATA TABLE 8
Telomeric Complex (Human Genome Expression Values)

| Official | | | | | | | | | | | | |
|---|---|---|---|---|---|---|---|---|---|---|---|---|
| Gene Symbol | GenBank | A | B | C | D | E | F | G | H | I | J | K |
| TNKS | A_23_P20927 | 1.1 | 1.3 | 1.1 | 1.5 | −1.3 | 2.2 | 2.2 | −1.9 | 1.9 | 0.0 | −3.8 |
| TNKS2 | AK314612 | −1.0 | 1.1 | 1.3 | −1.2 | −2.9 | 1.2 | 1.2 | −1.5 | 1.0 | 0.0 | −2.5 |
| TRF1 | A_23_P216149 | −1.0 | 1.3 | −1.4 | 1.4 | 1.0 | −2.1 | −2.2 | −1.3 | −2.3 | 0.1 | 1.1 |
| TIN2 | AK294119 | 1.1 | 1.1 | −1.4 | −1.4 | −1.0 | −2.6 | −2.9 | NA | −1.8 | 0.3 | NA |
| TPP1 | AK299703 | −1.1 | 1.0 | 1.1 | −1.3 | 1.1 | −1.4 | −1.3 | 1.0 | −1.0 | −0.1 | 2.1 |
| POT1 | NR_003103 | −1.0 | 1.2 | 1.0 | 1.0 | 1.1 | −1.3 | −1.1 | −1.3 | −1.0 | −0.2 | −0.3 |
| RAP1 | BC054490 | −1.1 | −1.1 | 1.3 | 1.0 | −1.1 | 1.3 | 1.3 | 1.1 | 1.0 | 0.0 | 0.0 |
| TRF2 | U95970 | 1.0 | −1.1 | 1.2 | −1.5 | 1.1 | −2.1 | −2.2 | NA | −2.3 | 0.1 | NA |
| TERT | DQ264729 | 1.1 | −1.3 | −1.0 | 1.3 | 1.0 | −1.0 | 1.4 | 1.0 | −1.1 | −2.5 | 2.1 |

A: Idebenone Alone Expression
B: Idebenone + UVB Expression
C: Coffee cherry Alone Expression
D: Coffee cherry + UVB Expression
E: UVB Alone Expression
F: Coffee cherry + H2O2 Expression (24 hours)
G: H2O2 Alone Expression (24 hours)
H: Coffee cherry + H2O2 Expression (8 hours)
I: H2O2 Alone Expression (8 hours)
J: Difference between Coffee cherry + H₂O₂ and H₂O₂ Alone Expression (24 hours)
K: Difference between Coffee cherry + H₂O₂ and H₂O₂ Alone Expression (8 hours)

DATA TABLE 9
(DNA Damage and Repair

| Official | | | | | | | | | | | | | |
|---|---|---|---|---|---|---|---|---|---|---|---|---|---|
| Gene Symbol | GenBank | A | B | C | D | E | F | G | H | I | J | K | L |
| ABL1 | X16416.1 | 1.0 | −1.0 | 1.2 | −1.0 | 1.1 | | −1.4 | −1.5 | 1.1 | 1.1 | 0.1 | 0.0 |
| AK3 | NM_016282.2 | 1.0 | 1.0 | 1.3 | 1.0 | 1.4 | | −1.4 | −1.4 | −1.5 | −1.2 | 0.0 | −0.2 |
| ANKRD17 | NM_198889.1 | −1.0 | 1.0 | 1.7 | −1.0 | 1.0 | | 1.6 | 1.1 | 1.3 | 1.5 | 0.5 | −0.2 |
| APEX1 | NM_001641.2 | 1.2 | −1.1 | −1.2 | 1.5 | 1.5 | | −1.0 | 1.0 | 1.1 | 1.1 | −2.1 | 0.0 |
| APEX2 | NM_014481.2 | −1.1 | 1.0 | −1.0 | 1.2 | 1.1 | | −1.4 | −2.3 | 1.1 | −1.2 | 0.9 | 2.3 |
| ATF2 | NM_001880.2 | 1.1 | −1.1 | 1.1 | 1.4 | −1.1 | | −1.2 | −1.1 | −1.2 | −1.8 | −0.1 | 0.6 |
| ATIC | NM_004044.4 | 1.0 | −1.0 | −1.0 | −1.1 | −1.0 | | 1.1 | 1.1 | 1.4 | 1.2 | 0.0 | 0.1 |
| ATM | U26455.1 | 1.2 | 1.9 | −1.1 | −1.2 | 1.0 | | 1.1 | −1.2 | 1.1 | 1.3 | 2.3 | −0.2 |
| ATR | NM_001184.2 | 1.2 | 1.2 | 1.0 | −1.2 | 1.1 | | −1.8 | −1.7 | −1.5 | −1.5 | 0.0 | 0.0 |
| ATRX | NM_000489.2 | 1.0 | −1.2 | −1.4 | −1.2 | 1.1 | | −1.7 | −1.4 | 2.0 | −1.9 | −0.2 | 3.9 |
| ATXN3 | NM_004993.3 | −1.1 | 1.8 | −1.2 | −1.4 | −1.2 | | 1.0 | 1.1 | 1.0 | 1.3 | 0.0 | −0.3 |
| B2M | NM_004048.2 | −1.0 | 1.3 | −1.4 | 1.0 | −1.0 | | 1.8 | 1.8 | −1.1 | 1.0 | 0.0 | −2.1 |
| BARD1 | NM_000465.1 | 1.4 | −1.1 | −1.3 | −1.2 | 1.1 | | 1.3 | 1.3 | 1.2 | −1.1 | 0.0 | 2.4 |
| BLM | NM_000057.1 | 1.0 | 1.2 | 1.3 | −1.0 | 7.4 | | 3.3 | 3.3 | 1.5 | 1.5 | 0.0 | 0.1 |
| BPNT1 | NM_006085.3 | −1.2 | 2.3 | −1.7 | −1.1 | 2.2 | | 1.1 | 1.1 | −1.4 | 1.2 | 0.0 | −2.6 |
| BRCA1 | Y08864.1 | −1.0 | 1.1 | 1.0 | 1.1 | 1.4 | | 1.7 | 1.8 | −1.3 | −1.1 | −0.1 | −0.2 |
| BRCA2 | NM_000059.1 | 1.1 | −1.1 | 1.0 | −1.1 | 1.1 | | 1.7 | 1.5 | −1.0 | 1.4 | 0.3 | −2.4 |
| BRIP1 | NM_032043.1 | 1.0 | 1.0 | 1.1 | 1.3 | 1.6 | | 2.2 | 2.4 | 1.7 | 1.7 | −0.2 | 0.0 |
| RPL13A | X56932.1 | −1.0 | −1.1 | 1.0 | −1.4 | 1.1 | | −1.0 | −1.0 | −1.0 | −1.0 | 0.0 | 0.0 |
| BTG2 | NM_006763.2 | 1.0 | −1.1 | −1.0 | 1.1 | 1.4 | | 1.7 | 1.3 | −1.2 | −1.8 | 0.5 | 0.6 |
| CCNH | NM_001239.2 | −1.1 | 1.2 | −1.6 | −1.4 | −1.0 | | −1.5 | 1.0 | 1.3 | 1.5 | −2.5 | −0.3 |
| CDA | NM_001785.1 | −1.5 | −1.0 | −1.2 | 1.4 | 1.0 | | 2.7 | 2.4 | −1.0 | 1.0 | 0.3 | −2.1 |
| CDK7 | NM_001799.2 | 1.0 | 1.0 | 1.1 | 1.2 | 1.2 | | 1.1 | 1.2 | −1.1 | −1.1 | −0.1 | 0.0 |
| CDKN1A | U03106.1 | 1.2 | −1.3 | 1.1 | 1.2 | −1.7 | | 1.7 | 1.0 | −1.2 | 1.2 | 0.7 | −2.4 |
| CDKN2A | L27211.1 | 1.0 | −1.0 | −1.1 | 1.2 | 1.1 | | −1.1 | −1.1 | 3.0 | −1.1 | −0.1 | 4.0 |
| CETN2 | NM_004344.1 | 1.1 | −1.1 | −1.1 | 1.2 | 2.3 | | −1.1 | −1.1 | −1.1 | −1.1 | −0.1 | 0.0 |
| CHEK1 | NM_001274.2 | −1.5 | 1.1 | 1.0 | −1.1 | −1.1 | | −3.2 | −3.7 | 1.2 | −2.3 | 0.6 | 3.5 |
| CHEK2 | NM_001005735.1 | 1.1 | −1.0 | −1.1 | 1.1 | 1.1 | | 1.1 | 1.0 | −1.2 | −1.3 | 0.1 | 0.1 |
| CIB1 | U85611.1 | 1.0 | −1.0 | 1.0 | 1.0 | 1.0 | | 1.4 | 1.3 | 1.1 | 1.0 | 0.1 | 0.0 |
| CIDEA | NM_001279.2 | 1.3 | −2.1 | −1.2 | −1.1 | −1.1 | | 1.0 | 1.2 | 1.9 | 1.5 | −0.2 | 0.4 |
| CIDEB | AF544398.1 | −1.2 | 1.1 | −1.3 | −1.1 | −1.1 | | 1.6 | 1.7 | −1.4 | 1.3 | −0.1 | −2.7 |
| CRY1 | NM_004075.2 | 1.0 | 2.1 | −1.2 | 1.0 | −1.1 | | 1.6 | 1.7 | −1.3 | −1.1 | −0.1 | −0.1 |
| CRY2 | NM_021117.1 | −1.1 | −1.4 | 1.0 | 1.8 | 1.4 | | 1.3 | 1.1 | 1.2 | −1.2 | 0.3 | 2.3 |
| CSF2 | M11220.1 | 2.4 | 1.0 | −1.1 | −1.0 | 1.2 | | 1.1 | 1.1 | 1.1 | 1.2 | 0.0 | −0.1 |
| CTPS | NM_001905.1 | 1.0 | 1.1 | 1.0 | 1.0 | 1.2 | | 2.8 | 2.9 | 3.4 | 3.0 | 0.0 | 0.4 |
| ALDOA | NM_000034.1 | 1.0 | −1.3 | 1.2 | −1.2 | 1.1 | | 1.4 | 1.3 | 1.4 | 1.2 | 0.1 | 0.1 |
| DCLRE1C | AJ296101.1 | 1.0 | 1.1 | 1.0 | 1.0 | 1.1 | | −1.5 | −1.5 | −1.6 | −1.6 | 0.0 | 0.0 |
| DDB1 | NM_001923.2 | 1.9 | 1.1 | −1.5 | −1.2 | 1.2 | | −1.1 | −1.1 | 1.2 | 1.1 | 0.0 | 0.1 |
| DDB2 | NM_000107.1 | 1.0 | −1.1 | −1.8 | 2.0 | 1.2 | | −2.6 | −3.3 | −2.7 | −3.0 | 0.7 | 0.3 |
| DDIT3 | S40706.1 | 1.1 | 1.1 | 1.2 | 1.3 | −1.0 | | 1.3 | 1.3 | −1.6 | −1.3 | 0.0 | −0.3 |
| K-ALPHA-1 | NM_006082.1 | | | | | | | 1.8 | 1.6 | 1.3 | 1.2 | 0.2 | 0.1 |
| DHFR | NM_000791.2 | 1.0 | −1.0 | −1.0 | 1.3 | 1.0 | | −2.2 | −1.6 | −1.4 | −1.1 | −0.6 | −0.3 |
| DKC1 | NM_001363.2 | 1.0 | 1.0 | 2.0 | 1.0 | 1.2 | | 1.1 | 1.1 | 1.4 | 1.4 | 0.0 | 0.0 |
| DMC1 | NM_007068.2 | 1.1 | −1.1 | 1.3 | −2.4 | 1.2 | | 1.2 | 1.1 | 1.2 | 1.1 | 0.1 | 0.1 |
| DPYS | NM_001385.1 | 1.1 | | | | | | 1.0 | −1.7 | −1.0 | −1.8 | 2.7 | 0.7 |
| DUT | NM_001948.3 | 1.5 | −1.1 | 1.1 | 1.1 | 1.0 | | −1.0 | 1.0 | 1.0 | 1.0 | −2.1 | 0.0 |
| E2F1 | NM_005225.1 | −1.5 | 1.1 | 1.1 | 1.1 | 1.1 | | 2.6 | 2.4 | 2.4 | 2.4 | 0.2 | 0.0 |
| E2F2 | NM_004091.1 | −1.1 | 1.1 | −1.0 | −1.2 | 1.0 | | 1.4 | −1.1 | 1.0 | 1.2 | 2.5 | −0.2 |
| E2F3 | NM_001949.2 | −1.5 | 1.3 | −1.5 | −1.5 | −1.1 | | −1.2 | −1.2 | 1.2 | 1.3 | 0.0 | −0.1 |
| ENTPD4 | NM_004901.1 | 1.1 | −1.3 | 1.0 | 1.0 | 1.0 | | 1.1 | −1.1 | −1.0 | −1.1 | 2.2 | 0.0 |
| ERCC1 | M28650.1 | 1.2 | −1.1 | 1.0 | −1.4 | −1.1 | | 1.1 | 1.1 | 1.9 | 1.8 | 0.0 | 0.1 |
| ERCC2 | NM_000400.2 | 1.0 | −1.0 | −1.1 | −1.0 | 1.1 | | 1.3 | 1.1 | 2.0 | 1.6 | 0.2 | 0.3 |
| ERCC3 | NM_000122.1 | −1.0 | −1.1 | −1.1 | −1.2 | −1.0 | | 1.1 | 1.2 | 1.2 | 1.3 | −0.1 | −0.1 |
| ERCC4 | U64315.1 | −1.1 | 1.9 | 1.0 | 1.0 | 1.0 | | −2.0 | −2.0 | −1.5 | −1.6 | 0.0 | 0.1 |
| ERCC5 | NM_000123.2 | 1.0 | −1.2 | −1.2 | 1.8 | 1.2 | | −1.2 | −1.2 | −1.2 | −1.1 | 0.0 | −0.1 |
| ERCC6 | NM_000124.1 | 1.4 | 1.1 | 1.0 | −1.3 | 1.0 | | −1.2 | −1.2 | −1.2 | −1.1 | 0.0 | −0.1 |
| ERCC8 | NM_000082.2 | −1.1 | 1.0 | 1.1 | 1.2 | 1.2 | | −1.0 | 1.1 | −1.3 | −1.4 | −2.2 | 0.0 |
| ACTB | NM_001101.2 | 1.1 | 1.2 | −1.1 | 1.2 | −1.1 | 2.2 | 1.1 | 1.1 | 1.1 | 1.0 | 0.0 | 0.0 |
| ERH | NM_004450.1 | 1.0 | 1.3 | −1.0 | −1.2 | 1.5 | | −1.4 | −1.9 | −1.3 | −1.1 | 0.5 | −0.2 |
| EXO1 | NM_006027.3 | 1.1 | −1.0 | 1.2 | 1.0 | −1.1 | | 3.3 | 3.4 | 2.4 | 2.2 | −0.1 | 0.2 |
| FANCA | NM_000135.2 | 1.0 | 1.1 | 1.6 | 1.4 | 1.2 | | 1.1 | 1.0 | 1.9 | 1.7 | 0.0 | 0.2 |
| FANCC | NM_000136.2 | −1.0 | −1.1 | −1.1 | 1.0 | −2.1 | | −1.5 | −1.2 | 1.1 | −1.1 | −0.3 | 2.2 |
| FANCF | NM_022725.2 | −1.1 | 3.3 | −1.2 | 1.9 | 1.1 | | −1.1 | −1.3 | −1.8 | −1.5 | 0.2 | −0.3 |
| FANCG | NM_004629.1 | 1.1 | 1.0 | −1.1 | 1.2 | −1.0 | | 2.1 | 2.1 | 1.8 | 1.6 | 0.0 | 0.2 |
| FEN1 | NM_004111.4 | −1.0 | −1.2 | 1.2 | 1.0 | 1.1 | | 3.6 | 3.2 | 1.7 | 1.5 | 0.4 | 0.2 |
| FPGS | NM_004957.4 | 1.2 | −1.1 | −1.7 | −1.2 | 1.3 | | 1.1 | 1.2 | 1.3 | 1.2 | 0.0 | 0.1 |
| GADD45A | NM_001924.2 | −1.1 | −1.2 | 1.0 | 1.7 | −1.0 | | 3.3 | 2.8 | 1.6 | 1.9 | 0.4 | −0.3 |
| GADD45G | NM_006705.2 | 1.0 | 1.1 | −1.0 | 1.0 | 1.3 | | 1.7 | 2.1 | 1.3 | 1.4 | −0.3 | −0.1 |
| GDA | NM_004293.2 | 1.0 | 1.0 | 1.3 | 1.2 | −1.3 | | −2.1 | −1.1 | −1.0 | 1.1 | −0.9 | −2.1 |
| GML | NM_002066.1 | −1.3 | −1.0 | −1.1 | 1.3 | 1.2 | | 1.2 | 1.1 | −1.2 | −1.3 | 0.1 | 0.2 |
| GMPR | NM_006877.2 | 1.0 | −1.4 | −1.6 | 1.1 | 1.2 | | −1.2 | 1.1 | 1.0 | 1.1 | −2.3 | −0.1 |
| GTF2H1 | NM_005316.2 | 1.1 | 1.0 | 1.1 | −1.2 | 1.0 | | 1.0 | 1.0 | −1.1 | −1.2 | 0.0 | 0.1 |
| GTF2H2 | NM_001515.2 | −1.2 | 1.0 | −1.4 | −1.4 | −1.1 | | 1.0 | 1.0 | −1.1 | −1.2 | 0.0 | 0.1 |
| GTF2H3 | NM_001516.3 | 1.0 | 1.1 | 1.2 | 1.0 | 1.3 | | 1.1 | 1.0 | 1.3 | 1.2 | 0.0 | 0.1 |
| GTF2H4 | NM_001517.4 | −1.4 | −1.1 | 1.0 | 1.0 | −1.1 | | 1.0 | 1.1 | 1.1 | 1.1 | −0.1 | 0.0 |
| GTSE1 | NM_016426.4 | −1.1 | −1.4 | −1.7 | 1.0 | 1.1 | | −2.4 | 1.3 | −1.3 | −4.5 | −3.7 | 3.2 |
| HSP90AB1 | NM_007355.2 | 1.1 | −1.4 | 1.1 | −1.6 | 1.3 | | −1.1 | −1.1 | −1.5 | −1.5 | 0.0 | 0.0 |
| HUS1 | NM_004507.2 | 1.0 | 1.3 | 1.0 | 1.1 | −1.1 | | 1.2 | 1.1 | 1.4 | 1.2 | 0.2 | 0.2 |
| IGHMBP2 | AB208802.1 | 1.1 | −1.4 | −1.1 | 1.3 | −1.1 | | 2.3 | 1.5 | 1.1 | 1.1 | 0.8 | 0.1 |
| IHPK3 | NM_054111.2 | 1.1 | 1.0 | 1.0 | 1.6 | −1.3 | | 1.3 | 1.4 | 1.0 | −1.0 | −0.1 | 2.0 |
| CHUK | NM_001278.2 | 1.0 | −1.2 | −1.1 | 1.0 | −1.1 | | 1.4 | 1.4 | 1.4 | 1.5 | −0.1 | −0.1 |
| INPPL1 | NM_001567.2 | −1.2 | −1.5 | 1.1 | 1.0 | 1.2 | | −1.0 | −1.0 | 1.2 | 1.2 | 0.0 | 0.0 |
| JUN | NM_002228.3 | 1.0 | 1.1 | 1.1 | −1.1 | −1.2 | | 1.9 | 2.2 | 1.8 | 2.0 | −0.3 | −0.2 |
| XRCC6BP1 | NM_033276.2 | 1.1 | 1.0 | 1.7 | 1.6 | −1.1 | | −1.2 | −1.3 | −2.8 | −2.6 | 0.0 | −0.2 |
| LIG1 | NM_000234.1 | 1.0 | −1.3 | 1.1 | −1.1 | 1.3 | | 4.6 | 4.3 | 2.4 | 2.3 | 0.3 | 0.1 |
| LIG3 | X84740.1 | 1.0 | −1.8 | 1.2 | 1.1 | −1.1 | | 1.1 | 1.0 | 3.6 | 3.2 | 0.0 | 0.4 |
| LIG4 | NM_002312.2 | 1.0 | −1.2 | 1.0 | −1.3 | −2.0 | | 1.0 | 1.1 | 1.1 | 1.2 | −0.1 | −0.1 |
| PPIE | AF042385.1 | 1.1 | −1.2 | −1.4 | −1.0 | −1.7 | | −2.3 | −2.1 | −1.5 | −1.0 | −0.2 | −0.5 |
| MAP2K6 | NM_002758.2 | 1.0 | 1.1 | 1.0 | 1.1 | −1.1 | | 1.0 | −1.6 | 1.1 | 1.0 | 2.6 | 0.1 |
| MAPK12 | NM_002969.2 | −1.0 | −1.5 | 1.0 | 1.0 | 1.0 | | −1.1 | 1.1 | 1.1 | 1.3 | −2.2 | −0.2 |
| MAPKAPK2 | NM_032960.2 | −1.4 | −1.1 | −1.2 | 1.1 | 1.1 | | 1.3 | 1.3 | 1.3 | 1.3 | 0.0 | 0.0 |
| MBD4 | NM_003925.1 | 1.0 | −1.0 | −1.0 | 1.0 | 1.1 | | 1.3 | 1.4 | 1.0 | 1.0 | −0.1 | 0.0 |
| MDM2 | NM_002392.2 | 1.1 | 1.7 | 1.5 | −1.0 | 1.0 | | 1.8 | 1.7 | 2.3 | 1.7 | 0.2 | 0.5 |
| MGMT | NM_002412.1 | −1.3 | −1.1 | −1.2 | −1.0 | 1.2 | | −1.3 | −1.2 | −1.0 | −1.1 | 0.0 | 0.1 |
| MLH1 | NM_000249.2 | 1.0 | 1.3 | 1.2 | 1.1 | −1.3 | | 1.1 | 1.1 | −1.0 | −1.1 | −0.1 | 0.1 |
| MLH3 | NM_014381.1 | 1.0 | −1.1 | −1.4 | −1.1 | 1.1 | | −1.5 | −1.7 | −1.1 | −1.1 | 0.2 | 0.0 |
| MMS19L | NM_022362.2 | | * | * | * | * | | −1.7 | −1.4 | −1.1 | −1.3 | −0.3 | 0.2 |
| MNAT1 | NM_002431.2 | 1.0 | 1.2 | 1.3 | 1.3 | −1.4 | | −1.1 | 1.2 | −1.1 | 1.1 | −2.3 | −2.2 |
| MPG | AY258284.1 | 1.0 | −1.0 | 1.0 | 1.0 | 1.2 | | −1.2 | −1.2 | 1.1 | 1.1 | 0.0 | 0.0 |
| MRE11A | AF022778.1 | 1.2 | 1.0 | 1.1 | −1.1 | 1.1 | | 1.0 | 1.2 | −1.7 | −1.7 | −0.2 | −0.1 |
| GAPDH | NM_002046.2 | −1.1 | −1.2 | 1.2 | 1.0 | 1.2 | −1.3 | 1.0 | 1.0 | −1.0 | −1.0 | 0.0 | 0.0 |
| MSH2 | NM_000251.1 | 1.0 | 1.2 | −1.1 | 1.3 | 1.3 | | 1.9 | 1.6 | 1.0 | 1.2 | 0.4 | −0.2 |
| MSH3 | NM_002439.2 | 1.1 | −1.0 | −1.2 | −1.1 | 1.3 | | −1.2 | 1.1 | −2.8 | −1.4 | −2.3 | −1.4 |
| MSH4 | NM_002440.2 | 1.0 | 1.3 | 1.2 | −2.5 | 1.1 | | 1.1 | −1.3 | 1.0 | 1.6 | 2.4 | −0.5 |
| MSH5 | NM_172166.1 | | * | * | * | * | | −1.4 | −1.2 | −1.0 | −1.1 | −0.2 | 0.1 |
| MSH6 | NM_000179.1 | 1.0 | 1.1 | 1.4 | −1.1 | 1.2 | | −1.4 | 1.0 | −2.3 | −1.7 | −2.4 | −0.5 |
| MTAP | NM_002451.3 | 1.1 | −1.2 | 1.6 | −1.1 | −1.2 | | 1.3 | 1.3 | 2.6 | 2.8 | 0.0 | −0.1 |
| MUTYH | NM_012222.1 | −1.0 | 1.0 | 1.2 | 1.0 | −1.1 | | 1.0 | 1.3 | 1.0 | 1.1 | −0.3 | −0.1 |
| N4BP2 | NM_018177.2 | 1.1 | 1.4 | 1.2 | 1.5 | 1.2 | | −2.2 | −2.2 | −1.6 | −1.4 | 0.0 | −0.2 |
| NBN | NM_002485.4 | −1.2 | 1.0 | 1.2 | −1.1 | −1.1 | | 1.2 | 1.1 | 1.6 | 1.3 | 0.1 | 0.3 |
| NEIL1 | NM_024608.1 | 1.5 | 1.0 | −1.1 | 1.2 | −1.1 | | −2.3 | −2.8 | 1.5 | −1.9 | 0.5 | 3.3 |
| NEIL2 | NM_145043.1 | 1.2 | 1.1 | 1.0 | 1.0 | −1.4 | | 1.2 | 1.2 | 1.3 | 1.2 | 0.0 | 0.1 |
| NEIL3 | NM_018248.1 | −1.0 | −1.0 | 1.0 | 1.2 | 1.0 | | 1.7 | 1.9 | −1.0 | −1.1 | −0.2 | 0.1 |
| NFKB1 | NM_003998.1 | −1.5 | −1.1 | 1.0 | −1.2 | 1.1 | 1.7 | −1.1 | −1.4 | 1.1 | −1.0 | 0.3 | 2.2 |
| NFKBIA | NM_020529.1 | 1.0 | 1.3 | −1.1 | −1.6 | 1.1 | | 1.5 | 1.5 | 1.4 | 1.6 | 0.0 | −0.2 |
| NP | NM_000270.1 | −1.2 | −1.1 | 1.0 | 1.0 | −1.0 | | 5.0 | 4.8 | 2.6 | 2.5 | 0.3 | 0.0 |
| NTHL1 | NM_002528.4 | 1.1 | −1.1 | −1.2 | −1.2 | 1.0 | | −1.0 | −1.1 | −1.2 | −1.3 | 0.1 | 0.1 |
| HK1 | M75126.1 | −1.1 | −1.1 | 1.0 | 1.4 | −1.1 | | 1.6 | 1.3 | 1.7 | 1.5 | 0.3 | 0.2 |
| NUDT1 | NM_198952.1 | 1.3 | −1.1 | −1.0 | 1.0 | −1.1 | | 1.4 | 1.2 | −1.3 | −1.3 | 0.2 | 0.0 |
| NUDT2 | NM_001161.3 | −1.0 | 1.0 | 1.0 | −1.4 | −1.1 | | −1.2 | −1.0 | −1.6 | −1.3 | −0.1 | −0.3 |
| OAS1 | D00068.1 | 1.3 | 1.0 | 1.0 | −1.1 | 1.0 | | −1.0 | 1.3 | 1.1 | 1.1 | −2.3 | 0.0 |
| OAS2 | M87284.1 | 1.2 | −1.1 | 1.0 | 1.0 | 1.3 | | −1.0 | −1.2 | −1.6 | −1.5 | 0.2 | −0.1 |
| HPRT1 | NM_000194.1 | 1.2 | 1.3 | 1.0 | 1.2 | −1.7 | | −2.1 | −2.0 | −1.4 | −1.3 | −0.1 | −0.2 |
| OAS3 | AB044545.1 | 1.0 | 1.0 | 1.0 | 1.1 | 2.7 | | −1.0 | 1.1 | 2.8 | 2.9 | −2.1 | −0.1 |
| ODC1 | NM_002539.1 | −1.0 | 1.0 | −1.1 | 1.0 | 1.1 | | 3.2 | 2.8 | 1.9 | 1.7 | 0.4 | 0.2 |
| OGG1 | AF003595.1 | −1.3 | 1.2 | 1.0 | −1.1 | 1.1 | | 1.4 | 1.4 | 1.3 | 1.2 | 0.0 | 0.1 |
| PAPSS1 | NM_005443.4 | −1.0 | −1.1 | −1.0 | −1.1 | −1.0 | | −1.8 | −1.6 | 1.0 | −1.1 | −0.1 | 2.1 |
| PAPSS2 | NM_001015880.1 | −1.0 | −1.1 | −1.1 | 1.6 | 1.1 | | −1.2 | 1.3 | 2.0 | 1.9 | −2.5 | 0.1 |
| PARP1 | J03473.1 | 1.3 | 1.0 | −1.0 | 1.3 | −1.1 | 3.3 | 1.1 | 1.1 | 1.8 | 1.7 | 0.0 | 0.1 |
| PARP3 | NM_005485.2 | −1.0 | −1.2 | −1.4 | −1.2 | 1.6 | | −1.7 | −1.6 | −1.3 | −1.2 | −0.1 | 0.0 |
| PCBP4 | NM_033008.1 | 1.0 | −1.0 | −1.1 | 1.0 | 1.2 | | 1.2 | 1.2 | 1.4 | 1.5 | 0.0 | −0.1 |
| PCNA | NM_002592.1 | 2.4 | 1.1 | 1.4 | 1.2 | −1.1 | | 1.8 | 1.7 | 1.1 | 1.1 | 0.1 | −0.1 |
| PDCD8 | NM_145812.1 | | * | * | * | * | | 1.0 | 1.2 | 1.4 | 1.7 | −0.2 | −0.3 |
| PINX1 | NM_017884.3 | 1.2 | 1.3 | 1.3 | 1.3 | −1.2 | | 2.2 | 2.3 | 1.5 | 1.6 | −0.1 | −0.1 |
| PMS1 | NM_000534.3 | 1.0 | 1.0 | 1.3 | 1.3 | −2.6 | | −1.8 | −2.0 | −4.2 | −3.6 | 0.2 | −0.6 |
| PMS2 | NM_000535.3 | 1.1 | −1.1 | −1.2 | −1.2 | −1.2 | | −1.1 | −1.5 | −1.1 | −1.5 | 0.4 | 0.4 |
| PMS2L1 | XM_380025.2 | 1.0 | 1.1 | 1.0 | 1.0 | 1.2 | | 1.3 | 1.3 | −1.2 | −1.1 | 0.0 | −0.1 |
| PMS2L3 | NM_001003686.1 | 1.0 | −1.0 | −1.2 | 1.8 | −12.5 | | 1.1 | 1.2 | −1.3 | −1.1 | −0.1 | −0.1 |
| PMS2L4 | D38500.1 | | * | * | * | * | | 3.1 | 2.2 | 1.1 | −2.2 | 0.9 | 3.2 |
| MDH1 | NM_005917.1 | −1.1 | 1.2 | 1.8 | 1.2 | 1.2 | | −1.4 | −1.2 | −1.4 | −1.5 | −0.1 | 0.1 |
| PNKP | NM_007254.2 | 1.0 | −1.3 | −1.0 | 1.6 | −1.0 | | 1.3 | 1.2 | 1.2 | 1.2 | 0.0 | 0.0 |
| POLB | NM_002690.1 | −1.0 | 1.0 | −2.7 | 1.0 | −1.3 | | 2.0 | 1.9 | 1.4 | 1.3 | 0.1 | 0.1 |
| POLD3 | NM_006591.1 | 1.0 | 1.1 | 1.4 | 1.0 | −1.0 | | 1.8 | 2.4 | 2.2 | 1.6 | −0.6 | 0.6 |
| POLE | NM_006231.2 | −1.1 | 1.3 | −1.1 | −1.1 | 1.3 | | −1.0 | 1.6 | 1.1 | 1.3 | −2.6 | −0.2 |
| POLI | NM_007195.1 | 1.0 | 1.5 | 1.0 | −1.7 | 1.3 | | −4.2 | −3.9 | −2.4 | −2.0 | −0.3 | −0.4 |
| POLL | NM_013274.2 | 1.0 | −1.2 | 1.3 | −2.2 | 1.2 | | −2.1 | −2.6 | −1.6 | −1.6 | 0.5 | 0.0 |
| POLR3H | NM_138338.2 | 1.0 | 1.1 | 1.0 | −1.2 | 1.9 | | 1.9 | 1.6 | 1.4 | 1.4 | 0.4 | 0.0 |
| PPP1R15A | NM_014330.2 | 1.0 | −1.1 | −1.1 | −1.0 | 1.2 | | 4.6 | 4.2 | 1.7 | 1.9 | 0.4 | −0.2 |
| PRKDC | NM_006904.6 | 1.0 | −1.1 | 1.0 | −1.0 | 1.3 | | 1.1 | 1.2 | 1.7 | 1.6 | −0.1 | 0.1 |
| PRPS2 | NM_002765.2 | −1.1 | −1.0 | 1.1 | 1.1 | −2.3 | | −1.2 | −1.0 | −1.3 | −1.1 | −0.2 | −0.2 |
| RAD1 | NM_002853.2 | −1.3 | 1.1 | 1.2 | −1.0 | −1.0 | | −1.4 | −1.3 | −1.0 | 1.0 | −0.2 | −2.1 |
| RAD17 | NM_002873.1 | −1.0 | −1.1 | 1.0 | 1.0 | 1.1 | | −1.2 | −1.1 | −1.2 | −1.2 | 0.0 | −0.1 |
| RAD18 | NM_020165.2 | 1.0 | 1.3 | −1.1 | 1.1 | −1.0 | | 1.9 | 1.9 | 1.9 | 1.6 | 0.0 | 0.3 |
| RAD21 | NM_006265.1 | 1.0 | 1.1 | 1.0 | 1.0 | 1.3 | | 1.1 | 1.1 | −1.1 | −1.1 | 0.0 | 0.0 |
| RAD23A | NM_005053.2 | 1.1 | −1.4 | 1.3 | 1.6 | 1.1 | | 1.4 | 1.4 | 1.0 | 1.1 | 0.0 | 0.0 |
| RAD23B | NM_002874.3 | 1.0 | −1.2 | 1.3 | 1.2 | −1.0 | | −1.9 | −2.1 | −1.4 | −1.4 | 0.2 | 0.0 |
| RAD50 | U63139.1 | | * | * | * | * | | −1.6 | −1.6 | −1.1 | −1.2 | 0.1 | 0.1 |
| RAD51 | D14134.1 | −1.4 | 1.0 | −1.1 | −1.7 | 1.2 | | −1.1 | 1.2 | 1.1 | 1.2 | −2.2 | −0.1 |
| RAD51C | NM_058216.1 | −1.5 | 1.0 | 1.1 | 1.0 | −1.0 | | 1.2 | 1.3 | −1.3 | −1.4 | −0.1 | 0.0 |
| RAD51L1 | NM_002877.4 | 1.0 | −1.2 | 1.5 | 1.5 | 1.2 | | 2.0 | 1.8 | −1.4 | −1.1 | 0.2 | −0.3 |
| RAD51L3 | NM_133628.1 | 1.0 | −1.4 | −1.1 | −1.3 | 1.0 | | 1.2 | 1.3 | 1.3 | 1.3 | −0.1 | 0.0 |
| RAD52 | U12134.1 | 1.4 | 1.2 | 1.0 | 1.0 | 1.1 | | −1.4 | −1.6 | 1.9 | 1.2 | 0.1 | 0.7 |
| RAD54B | NM_134434.1 | 1.1 | 1.2 | −1.2 | −1.4 | 1.1 | | 2.9 | 2.9 | 1.4 | 1.5 | 0.0 | 0.0 |
| RAD54L | NM_003579.2 | −1.0 | −1.1 | 1.2 | 1.0 | 1.2 | | 4.3 | 5.0 | 2.0 | 2.0 | −0.7 | 0.1 |
| RAD9A | NM_004584.2 | 1.0 | −1.1 | −1.4 | 1.3 | 1.6 | | −3.5 | −3.7 | −2.0 | −1.6 | 0.2 | −0.4 |
| RBBP8 | NM_002894.2 | −1.1 | 1.3 | 1.1 | 1.0 | −1.2 | | 1.5 | 1.8 | 1.5 | 1.6 | −0.2 | −0.1 |
| RELA | L19067.1 | 1.0 | −1.0 | −1.5 | 1.0 | 1.1 | | 1.3 | 1.2 | 1.1 | −1.0 | 0.1 | 2.2 |
| REV1L | NM_016316.1 | | * | * | * | * | | 2.5 | 1.5 | −1.0 | −1.1 | 1.1 | 0.0 |
| REV3L | NM_002912.1 | −1.1 | −1.0 | 1.2 | 1.0 | 1.0 | | −1.8 | −1.5 | 1.6 | 1.1 | −0.3 | 0.5 |
| YWHAZ | M86400.1 | −1.0 | −1.1 | 1.0 | 1.0 | −1.3 | | −1.2 | −1.1 | −1.2 | −1.2 | −0.1 | 0.0 |
| RFC1 | NM_002913.3 | 1.0 | −1.0 | 1.0 | 1.0 | 1.0 | | −1.3 | −1.3 | −1.1 | −1.1 | 0.0 | 0.0 |
| RPA1 | NM_002945.2 | −1.1 | 1.1 | −1.0 | 1.0 | 1.1 | | −1.7 | −1.6 | 1.2 | 1.1 | −0.1 | 0.1 |
| RPA3 | NM_002947.3 | 1.1 | −1.0 | 1.1 | 1.8 | 1.0 | | 1.5 | −1.5 | −1.3 | 1.1 | 3.0 | −2.4 |
| RPS27A | NM_002954.3 | 1.0 | 1.3 | 1.1 | 1.0 | 1.2 | | −1.0 | 1.0 | 1.0 | 1.1 | −2.0 | 0.0 |
| SEMA4A | NM_022367.2 | 1.0 | 1.1 | 1.0 | 1.1 | 2.3 | | 2.8 | 3.0 | 5.3 | 4.4 | −0.3 | 0.9 |
| SESN1 | NM_014454.1 | 1.0 | −1.1 | −1.3 | −1.3 | 1.3 | | 1.6 | 1.2 | −1.0 | −1.3 | 0.4 | 0.3 |
| SLC23A1 | NM_005847.3 | −1.0 | 1.0 | 1.1 | −1.1 | 1.1 | | −1.7 | −1.3 | −1.3 | 1.2 | −0.4 | −2.6 |
| SLC23A2 | NM_203327.1 | 1.0 | 1.2 | 1.2 | −1.2 | 1.2 | | −1.0 | 1.1 | −1.0 | −1.0 | −2.1 | 0.0 |
| SLK | NM_014720.1 | −1.0 | 1.1 | 1.0 | −1.4 | 1.1 | | −1.1 | −1.1 | −1.1 | 1.1 | 0.0 | −2.2 |
| SMC1L1 | NM_006306.2 | | * | * | * | * | | −1.4 | 1.2 | 1.1 | −1.1 | −2.6 | 2.1 |
| SMUG1 | NM_014311.1 | 1.2 | −1.1 | 1.0 | 1.1 | −1.1 | | 1.3 | 1.3 | −1.1 | −1.1 | 0.0 | 0.0 |
| SUMO1 | NM_003352.4 | 1.2 | −1.2 | −1.6 | 1.1 | 1.3 | | −1.2 | −1.0 | −1.0 | −1.1 | −0.2 | 0.0 |
| RPS9 | NM_001013.2 | 1.1 | 1.0 | −1.1 | 1.0 | −1.0 | | 1.2 | 1.3 | 1.1 | −1.2 | −0.1 | 2.3 |
| TDG | NM_003211.3 | −1.1 | 1.2 | 1.0 | −1.1 | 2.1 | | −1.7 | −2.7 | −1.3 | 1.0 | 1.1 | −2.3 |
| TELO2 | A_23_P206237 | 1.1 | −1.1 | −1.4 | 1.0 | 1.0 | 1.6 | 1.8 | 1.8 | 2.0 | 2.0 | 0.0 | 0.0 |
| TEP1 | NM_007110.2 | 1.1 | 1.1 | 1.5 | 1.2 | 1.1 | | −1.9 | −1.7 | −1.3 | 1.0 | −0.2 | −2.3 |
| TERF1 | U40705.1 | 1.0 | 1.4 | −1.0 | −1.1 | 1.3 | | −1.4 | −1.3 | −1.5 | −1.4 | −0.1 | −0.1 |
| TERF2 | NM_005652.2 | 1.0 | −1.1 | 1.2 | −1.5 | 1.1 | | 2.2 | 1.6 | 1.3 | 1.3 | 0.6 | 0.0 |
| TERF2IP | NM_018975.2 | −1.1 | −1.1 | 1.3 | 1.0 | −1.1 | 1.2 | 1.3 | 1.3 | 1.0 | 1.0 | 0.1 | 0.0 |
| TERT | AF018167.1 | 1.1 | −1.3 | −1.0 | 1.3 | 1.0 | | −1.0 | 1.4 | 1.0 | −1.1 | −2.5 | 2.1 |
| TINF2 | NM_012461.1 | 1.1 | 1.1 | −1.4 | −1.4 | −1.0 | | −1.0 | −1.1 | −1.2 | −1.1 | 0.0 | −0.1 |
| TK1 | NM_003258.1 | 1.1 | −1.2 | 1.4 | −1.2 | 1.0 | | −1.0 | −1.2 | 1.4 | 1.5 | 0.2 | −0.1 |
| TK2 | NM_004614.2 | −1.2 | −1.2 | 1.0 | −1.0 | 1.1 | | −2.0 | −1.8 | −3.8 | −4.1 | −0.2 | 0.2 |
| TNKS | NM_003747.1 | 1.1 | 1.6 | 1.3 | 1.2 | 1.0 | | 2.2 | 2.2 | 1.8 | 1.9 | 0.0 | −0.2 |
| TNKS1BP1 | NM_033396.1 | 1.0 | −1.3 | 1.7 | 2.1 | 2.4 | | −2.5 | −2.6 | −1.7 | −1.2 | 0.1 | −0.5 |
| TNKS2 | NM_025235.2 | −1.0 | 1.1 | 1.3 | −1.2 | −2.9 | | 1.2 | 1.2 | 1.0 | 1.0 | 0.0 | 0.0 |
| SDS | NM_006843.1 | 1.0 | 1.2 | 1.0 | 1.1 | 1.4 | | 1.4 | 1.5 | −1.2 | 1.4 | −0.1 | −2.6 |
| TOP1 | NM_003286.2 | 1.2 | −1.4 | 1.0 | 1.0 | 1.1 | | 1.9 | 1.7 | 2.2 | 1.5 | 0.2 | 0.7 |
| TOP2A | NM_001067.2 | 1.0 | −1.3 | −1.4 | −1.1 | 2.6 | | −1.1 | 1.7 | −1.4 | −1.4 | −2.7 | −0.1 |
| TOP2B | NM_001068.2 | −1.2 | 1.1 | 1.2 | −1.0 | −1.0 | | −1.6 | −1.6 | −1.0 | −1.0 | −0.1 | 0.0 |
| TOP3A | NM_004618.3 | 1.0 | −1.0 | −1.1 | −1.0 | −2.2 | | 2.3 | 2.2 | 1.6 | 1.8 | 0.2 | −0.2 |
| TOP3B | NM_003935.3 | −1.0 | −1.2 | 1.7 | −2.7 | −1.0 | −1.1 | −1.8 | −1.3 | 1.5 | 1.7 | −0.5 | −0.2 |
| TP53 | AF307851.1 | −1.1 | −1.3 | −1.0 | 1.2 | 1.2 | | −2.1 | −2.0 | −1.5 | −1.5 | −0.2 | 0.0 |
| TP73 | NM_005427.1 | −1.0 | −1.1 | 1.1 | 1.1 | 1.1 | | 1.3 | 1.3 | 1.1 | 1.1 | 0.1 | 0.0 |
| TPMT | NM_000367.2 | −1.0 | −1.0 | 1.2 | 1.0 | −1.2 | | −1.5 | 1.2 | −1.1 | −1.2 | −2.7 | 0.1 |
| TREX1 | NM_033628.2 | −1.2 | −1.1 | −1.4 | −1.0 | −1.0 | | 1.1 | 1.0 | −1.1 | −1.2 | 0.0 | 0.0 |
| TREX1 | NM_130384.1 | −1.2 | −1.1 | −1.4 | −1.0 | −1.0 | | 1.1 | 1.0 | −1.1 | −1.2 | 0.0 | 0.0 |
| TREX2 | AF267739.1 | −1.2 | 1.7 | 1.0 | 1.0 | −1.2 | | −2.1 | −2.2 | −2.5 | −2.3 | 0.1 | −0.2 |
| TYMS | NM_001071.1 | 1.0 | 1.1 | 1.0 | 2.0 | 1.3 | | −1.6 | −1.3 | −1.3 | −1.0 | −0.3 | −0.2 |
| UBE2A | M74524.1 | 1.1 | 1.1 | −1.0 | −1.0 | 1.1 | | 1.4 | 1.4 | −1.2 | −1.1 | 0.1 | 0.0 |
| UBE2B | NM_003337.2 | 1.1 | −1.1 | 1.0 | 1.0 | 1.1 | | 3.0 | 1.8 | 1.6 | −1.2 | 1.2 | 2.8 |
| UBE2I | U45328.1 | 1.0 | −1.3 | −1.2 | −1.1 | 1.2 | | −2.1 | −1.7 | −1.0 | −1.3 | −0.4 | 0.3 |
| UBE2N | NM_003348.3 | 1.0 | −1.1 | −1.3 | 2.8 | −1.1 | | 1.5 | 1.5 | 1.4 | 1.4 | 0.0 | 0.0 |
| UBE2V2 | NM_003350.2 | −1.1 | −1.0 | −1.3 | −1.1 | −2.4 | | −2.2 | −1.9 | −2.0 | −1.5 | −0.3 | −0.4 |
| UNG | X15653.1 | 1.0 | −1.3 | 1.2 | 1.1 | −1.2 | | 1.1 | 1.0 | 1.9 | 1.7 | 0.1 | 0.2 |
| UNG2 | NM_021147.2 | | * | * | * | * | | 1.1 | 1.0 | 1.9 | 1.7 | 0.1 | 0.2 |
| UPP1 | NM_181597.1 | −1.1 | 1.1 | 1.1 | −1.2 | 1.1 | | 3.1 | 2.5 | 3.3 | 3.0 | 0.7 | 0.2 |
| WRN | NM_000553.2 | 2.4 | 1.1 | −1.0 | 1.3 | 1.1 | | −1.1 | −1.0 | 1.4 | 1.6 | −0.1 | −0.2 |
| XAB2 | NM_020196.2 | 1.0 | 1.0 | −1.1 | −1.2 | 1.1 | | −1.1 | −1.3 | −1.4 | −1.1 | 0.2 | −0.3 |
| XPA | NM_000380.2 | −1.4 | 1.1 | −1.2 | −1.6 | 1.2 | | −1.6 | −1.2 | −1.8 | −1.4 | −0.4 | −0.5 |
| XPC | NM_004628.3 | −1.4 | −1.3 | 1.3 | 1.5 | 1.6 | | −1.8 | −1.9 | −1.5 | −1.4 | 0.1 | −0.1 |
| XRCC1 | NM_006297.1 | −1.4 | −1.3 | −1.2 | −1.1 | 1.2 | | 1.2 | 1.3 | 1.1 | 1.0 | −0.1 | 0.0 |
| TFRC | NM_003234.1 | 1.2 | 1.0 | 1.2 | 1.0 | 1.1 | | −2.0 | −2.0 | 1.6 | −1.8 | 0.1 | 3.4 |
| XRCC2 | NM_005431.1 | −1.2 | 1.0 | 1.2 | 1.0 | 1.1 | | 2.1 | 2.6 | 1.9 | 1.5 | −0.5 | 0.4 |
| XRCC3 | NM_005432.2 | 1.1 | 1.2 | 1.6 | 1.3 | −2.3 | | 2.1 | 1.4 | 1.9 | 2.2 | 0.6 | −0.3 |
| XRCC4 | NM_022550.1 | 1.0 | −1.1 | 1.0 | −1.0 | −1.0 | | −1.0 | 1.1 | −1.5 | −1.3 | −2.1 | −0.2 |
| XRCC5 | NM_021141.2 | 1.6 | −1.2 | −1.3 | 1.0 | 1.1 | | −1.2 | −1.1 | 1.1 | 1.3 | −0.1 | −0.3 |
| XRCC6 | S38729.1 | −1.0 | −1.5 | −1.6 | −1.3 | −1.1 | | 1.2 | 1.1 | −1.0 | −1.1 | 0.1 | 0.1 |
| ZAK | NM_133646.1 | 1.0 | 1.4 | 1.0 | −3.5 | −2.3 | | 1.8 | 1.7 | 1.7 | 1.7 | 0.1 | 0.0 |
| ZNRD1 | X16416.1 | −1.6 | 1.1 | 1.0 | −1.0 | 1.2 | | 1.1 | 1.1 | −1.0 | 1.0 | 0.0 | −2.0 |

A: Idebenone Alone Expression
B: Idebenone + UVB Expression
C: Coffee cherry Alone Expression
D: Coffee cherry + UVB Expression
E: UVB Alone Expression
F: Green tea + UVB Expression
G: Coffee cherry + H2O2 Expression (24 hours)
H: H2O2 Alone Expression (24 hours)
I: Coffee cherry + H2O2 Expression (8 hours)
J: H2O2 Alone Expression (8 hours)
K: Difference between Coffee cherry + H2o2 and H202 Alone Expression (24 hours)
L: Difference between Coffee cherry + H2o2 and H202 Alone Expression (8 hours)

DATA TABLE 10
Custom Array I Genes

| Official Gene | | | | | | | | | | | | | |
|---|---|---|---|---|---|---|---|---|---|---|---|---|---|
| Symbol | GenBank | A | B | C | D | E | F | G | H | I | J | K | L |
| PARP1 | NM_001618 | 1.3 | 1.0 | −1.0 | 1.3 | −1.1 | 4.4 | 1.1 | 1.1 | 1.8 | 1.7 | 0.0 | 0.1 |
| IGF1 | NM_000618 | 1.0 | 1.0 | 1.2 | 1.4 | 1.2 | −1.2 | −1.0 | −1.0 | −1.4 | −1.3 | 0.0 | 0.0 |
| SHC1 | NM_003029 | 1.2 | −1.2 | −1.4 | −1.0 | −1.1 | 1.0 | 1.2 | 1.3 | 1.3 | 1.4 | −0.1 | −0.1 |
| IL1A | NM_000575 | −1.0 | 1.0 | 1.1 | 1.0 | −1.0 | −5.3 | 1.1 | −1.1 | 1.1 | 1.1 | 2.2 | −0.1 |
| NADSYN1 | NM_018161 | 1.2 | −1.1 | −1.3 | −1.1 | −1.5 | 3.4 | −1.4 | −1.4 | 1.3 | 1.2 | 0.0 | 0.1 |
| GAPDH | NM_002046 | −1.1 | −1.4 | −1.5 | 1.1 | 1.1 | 1.1 | 1.0 | 1.0 | −1.0 | −1.0 | 0.0 | 0.0 |
| PARP2 | NM_005484 | 1.3 | 1.2 | 1.1 | 1.0 | −1.5 | −6.5 | 1.9 | 2.0 | −1.1 | −1.1 | −0.1 | 0.0 |
| IGF2 | NM_000612 | 1.0 | 1.0 | −1.2 | 1.0 | 1.3 | 1.1 | 1.2 | 1.9 | −2.0 | −1.3 | −0.6 | −0.7 |
| IFI44 | NM_006417 | −1.6 | 1.0 | 1.0 | −1.6 | 1.1 | 2.7 | −1.1 | 1.1 | −2.0 | −1.4 | −2.2 | −0.6 |
| IL6 | NM_000600 | −1.0 | 1.5 | −1.3 | 1.0 | 1.0 | −1.5 | −1.2 | −1.0 | 2.6 | 2.8 | −0.2 | −0.1 |
| BAX | NM_004324 | 1.3 | 1.3 | 1.3 | −1.3 | 1.0 | 2.3 | −1.5 | −1.3 | −1.0 | 1.0 | −0.2 | −2.0 |
| ACTB | NM_001101 | 1.0 | −1.1 | −1.2 | 1.4 | −1.0 | −1.8 | 1.1 | 1.1 | 1.1 | 1.0 | 0.0 | 0.0 |
| PPARG | NM_015869 | −1.0 | −1.2 | 1.5 | −1.0 | −1.1 | −1.7 | −1.3 | −1.2 | −1.1 | −1.1 | −0.1 | 0.0 |
| CLK1 | NM_004071 | 1.0 | −1.1 | 1.0 | −1.3 | 1.1 | 1.7 | 1.6 | 1.8 | −1.1 | 1.2 | −0.2 | −2.2 |
| CREBBP | NM_004380 | −1.0 | 1.3 | −1.2 | 1.0 | −1.1 | −1.8 | −1.7 | −2.3 | −2.4 | −1.9 | 0.5 | −0.5 |
| IL10 | NM_000572 | 1.1 | 1.1 | 1.2 | 1.0 | −1.5 | −2.8 | 3.0 | 2.2 | 1.1 | 1.2 | 0.8 | 0.0 |
| COX1 | NP_536845 | NF | NF | NF | NF | NF | −1.1 | 1.9 | 1.8 | 1.5 | 1.4 | 0.1 | 0.2 |
| HPRT1 | NM_000194 | 1.2 | 1.3 | 1.0 | 1.2 | −1.7 | 1.4 | −2.1 | −2.0 | −1.4 | −1.3 | −0.1 | −0.2 |
| APOE | NM_000041 | 1.0 | −1.2 | 1.0 | 1.2 | 1.1 | −1.6 | 1.2 | 1.4 | −1.2 | −1.7 | −0.2 | 0.5 |
| TERT | NM_198255 | 1.1 | −1.3 | −1.0 | 1.3 | 1.0 | 1.2 | −1.0 | 1.4 | 1.0 | −1.1 | −2.5 | 2.1 |
| CYP19A1 | NM_000103 | 1.4 | 1.0 | 1.3 | 1.9 | −1.7 | −4.5 | 1.4 | 1.9 | −2.5 | −1.4 | −0.6 | −1.1 |
| TNF | NM_000594 | 1.1 | −1.4 | 1.1 | 1.0 | −1.1 | −18.4 | −1.7 | −1.7 | 1.0 | 1.3 | 0.0 | −0.3 |
| PTGS2 | NM_000963 | −1.2 | 1.3 | 1.5 | −1.3 | 1.1 | −2.0 | 1.9 | 1.9 | 6.0 | 5.8 | 0.1 | 0.2 |
| HIGX1A | SA_00105 | NF | NF | NF | NF | NF | −23.5 | −1.1 | −1.1 | −1.2 | −1.1 | 0.1 | −0.1 |
| HLA-DRA | NM_019111 | 1.0 | 1.0 | 1.4 | −1.4 | 1.2 | −16.6 | −1.2 | 1.0 | 1.0 | 1.2 | −2.2 | −0.1 |
| TEP1 | NM_007110 | 1.0 | −1.3 | −1.1 | −1.3 | 1.3 | 1.1 | −1.9 | −1.7 | −1.3 | 1.0 | −0.2 | −2.3 |
| BCL2 | NM_000633 | 1.0 | 1.2 | −1.2 | −1.1 | 1.0 | 3.5 | −1.1 | 1.0 | 1.8 | 1.8 | −2.2 | 0.0 |
| HSPA1A | NM_005345 | 1.4 | −1.0 | −1.1 | 1.1 | 1.0 | 2.1 | 3.4 | 4.4 | −1.0 | 1.1 | −1.0 | −2.2 |
| HSPA1B | NM_005346 | NF | NF | NF | NF | NF | 3.5 | 4.4 | 5.2 | 1.2 | 1.3 | −0.8 | −0.1 |
| DDC | NM_000790 | 1.0 | 1.0 | 1.2 | −1.1 | −1.1 | −12.2 | 1.2 | −1.2 | −1.3 | 1.0 | 2.4 | −2.4 |
| SIRT1 | NM_012238 | −1.2 | 1.1 | 1.4 | 1.0 | 1.1 | 1.3 | 1.4 | 1.3 | −1.0 | 1.1 | 0.1 | −2.2 |
| KRAS | NM_004985 | −1.2 | −1.1 | 1.2 | −1.1 | 1.2 | 1.6 | −1.8 | −2.3 | 1.5 | −2.2 | 0.5 | 3.7 |
| SOD1 | NM_000454 | −1.0 | 1.0 | −1.1 | −1.0 | 1.1 | 1.2 | −1.0 | −1.0 | −1.2 | −1.1 | 0.0 | 0.0 |
| HSPA1L | NM_005527 | 1.0 | −1.1 | 1.0 | 1.0 | 1.3 | −3.9 | 1.4 | 1.5 | 1.5 | 1.9 | −0.1 | −0.4 |
| ACE | NM_000789 | −1.0 | −1.1 | −1.8 | 1.1 | −1.1 | 2.6 | −2.9 | −2.3 | −1.2 | −1.0 | −0.6 | −0.2 |
| TP53 | NM_000546 | −1.1 | −1.3 | −1.0 | 1.2 | 1.2 | −1.2 | −2.1 | −2.0 | −1.5 | −1.5 | −0.2 | 0.0 |
| SOD2 | NM_000636 | 1.1 | 1.1 | −1.1 | −1.2 | −1.0 | 1.3 | 2.8 | 2.3 | 1.6 | 1.5 | 0.5 | 0.1 |
| CASP9 | NM_001229 | −1.0 | 1.0 | 1.2 | 1.7 | −1.0 | 2.5 | 1.0 | 1.2 | 1.1 | 1.3 | −0.1 | −0.2 |
| CCL4L1 | NM_001001435 | NF | NF | NF | NF | NF | −1.7 | 1.2 | −1.1 | 1.9 | −1.1 | 2.3 | 3.0 |
| GH1 | NM_000515 | 1.0 | 1.3 | −1.6 | −1.2 | 1.2 | −8.0 | 1.3 | 1.1 | −1.2 | −1.3 | 0.2 | 0.1 |
| MAPK14 | NM_001315 | 1.0 | −1.4 | −1.1 | 1.0 | −1.2 | 2.3 | −1.3 | −1.3 | 1.1 | 1.1 | 0.0 | −0.1 |
| NOS2A | NM_000625 | 1.0 | 1.1 | 1.0 | −1.6 | −1.1 | −10.6 | −1.3 | −2.0 | 1.1 | −1.3 | 0.7 | 2.4 |
| CASP2 | NM_032982 | 1.0 | −1.3 | 1.3 | 1.0 | 1.3 | 2.1 | 1.1 | 1.0 | −1.2 | −1.4 | 0.0 | 0.2 |
| NFKB1 | NM_003998 | −1.5 | −1.1 | 1.0 | −1.2 | 1.1 | 2.6 | −1.1 | −1.4 | 1.1 | −1.0 | 0.3 | 2.2 |

NF: not found
A: Idebenone Alone Expression
B: Idebenone + UVB Expression
C: Coffee cherry Alone Expression
D: Coffee cherry + UVB Expression
E: UVB Alone Expression
F: Green tea + UVB Expression
G: Coffee cherry + H2O2 Expression (24 hours)
H: H2O2 Alone Expression (24 hours)
I: Coffee cherry + H2O2 Expression (8 hours)
J: H2O2 Alone Expression (8 hours)
K: Difference between Coffee cherry + H2O2 and H2O2 Alone Expression (24 hours)
L: Difference between Coffee cherry + H2O2 and H2O2 Alone Expression (8 hours)

DATA TABLE 11
Custom Array II Genes

| Official | | | | | | | | | | | |
|---|---|---|---|---|---|---|---|---|---|---|---|
| Gene | | | | | | | | | | | |
| Symbol | GenBank | A | B | C | D | E | F | G | H | I | J |
| ACE | NM_000789 | −2.9 | −2.3 | −1.2 | −1.0 | −1.0 | −1.1 | −1.8 | 1.1 | −1.1 | 2.6 |
| ACTB | NM_001101 | 1.0 | 1.1 | 1.1 | 1.0 | 1.0 | −1.1 | −1.2 | 1.4 | −1.0 | −1.8 |
| APOE | NM_000041 | −1.2 | 1.4 | −1.2 | −1.7 | 1.0 | −1.2 | 1.0 | 1.2 | 1.1 | −1.6 |
| BAX | NM_004324 | −1.5 | −1.3 | −1.0 | 1.0 | 1.3 | 1.3 | 1.3 | −1.3 | 1.0 | 2.3 |
| BCL2 | NM_000633 | −1.3 | 1.0 | 1.8 | 1.8 | 1.2 | 1.6 | 1.3 | 1.0 | 1.1 | 3.5 |
| BCL2L1 | NM_138578 | −1.7 | 1.2 | 1.1 | −1.2 | 1.0 | 1.0 | 1.0 | −1.1 | 2.7 | NA |
| BMP2 | NM_001200 | 10.3 | 8.6 | 8.4 | 10.5 | 1.1 | −1.7 | −1.1 | 1.2 | 1.0 | NA |
| CASP2 | NM_032982 | −1.5 | 1.0 | −1.2 | −1.4 | 1.0 | −1.3 | 1.3 | 1.0 | 1.3 | 2.1 |
| CASP9 | NM_001229 | −1.2 | 1.2 | 1.1 | 1.3 | −1.0 | 1.0 | 1.2 | 1.7 | −1.0 | 2.5 |
| CCL4L1 | NM_001001435 | 1.2 | −1.1 | 1.9 | −1.1 | 1.0 | 1.2 | −1.1 | 1.7 | 1.0 | −1.7 |
| CDKN2A | NM_000077 | −2.2 | −1.1 | −1.1 | −1.1 | 1.0 | −1.0 | −1.6 | 1.0 | 1.2 | NA |
| CLK1 | NM_004071 | 1.6 | 1.8 | −1.1 | 1.2 | 1.0 | −1.1 | 1.0 | −1.3 | 1.1 | 1.7 |
| COL1A1 | NM_000088 | −2.0 | −1.1 | 1.0 | 1.0 | 1.0 | −1.0 | 1.4 | 1.0 | −1.8 | NA |
| COL3A1 | NM_000090 | −4.4 | −5.3 | −4.8 | −5.7 | −1.1 | −1.2 | 1.2 | −2.6 | 1.1 | NA |
| COX1 | NP_536845 | 1.9 | 1.8 | 1.5 | 1.4 | 1.0 | 1.5 | 1.0 | −1.2 | 1.2 | −1.1 |
| CREBBP | NM_004380 | −1.7 | −2.3 | −2.4 | −1.9 | 1.0 | −1.1 | −1.1 | 1.2 | 1.2 | −1.8 |
| CRP | NM_000567 | −1.1 | −1.9 | −1.6 | −1.9 | −1.0 | 1.0 | 1.5 | 1.0 | 1.1 | NA |
| CYP19A1 | NM_000103 | −1.0 | 1.9 | −2.5 | −1.4 | 1.4 | 1.0 | 1.3 | 1.9 | −1.7 | −4.5 |
| DDC | NM_000790 | −1.5 | −1.2 | −1.3 | 1.0 | 1.0 | 1.0 | 1.2 | −1.1 | −1.1 | −12.2 |
| DUSP2 | NM_004418 | 22.8 | 19.8 | 9.5 | 8.1 | 1.0 | 1.1 | 1.2 | 1.1 | 1.2 | NA |
| EGF | NM_001963 | −1.2 | 1.2 | 1.7 | −1.2 | 1.0 | 1.0 | −1.1 | 1.3 | 1.1 | NA |
| EGR2 | NM_000399 | −6.6 | −2.5 | −1.8 | 1.0 | 1.0 | 1.7 | −1.3 | −1.9 | −1.6 | NA |
| FOS | NM_005252 | 1.4 | 1.3 | 1.1 | −1.0 | 1.0 | −1.4 | 1.4 | 1.2 | −1.1 | NA |
| FOXO3 | NM_001455 | −1.5 | −1.5 | −1.7 | −1.7 | 1.0 | −1.3 | 1.4 | 1.3 | 1.2 | NA |
| GADPH | NM_002046 | 1.3 | 1.5 | 1.3 | 1.1 | 1.3 | −1.2 | 1.1 | 1.0 | 1.0 | NA |
| GCH1 | NM_000161 | 2.4 | 2.2 | 1.6 | 1.4 | 1.0 | −1.7 | 1.6 | 1.9 | 1.5 | 1.1 |
| GH1 | NM_000515 | −1.0 | 1.1 | −1.2 | −1.3 | 1.0 | 1.3 | −1.6 | −1.2 | 1.2 | −8.0 |
| GPX1 | NM_000581 | −1.3 | −1.3 | −1.1 | −1.1 | 1.0 | −1.1 | −1.1 | 1.1 | 2.0 | NA |
| HBEGF | NM_001945 | −1.1 | −1.1 | 1.1 | −1.1 | 1.0 | 1.8 | 1.2 | 1.0 | 1.0 | NA |
| HGDC | SA_00105 | 1.2 | 2.8 | −1.7 | 1.2 | 1.2 | 1.1 | 1.6 | 1.0 | −1.6 | NA |
| HLA-DRA | NM_019111 | −1.2 | 1.0 | 1.0 | 1.2 | 1.0 | 1.0 | 1.4 | −1.4 | 1.2 | −16.6 |
| HMOX1 | NM_002133 | 34.2 | 22.3 | 6.2 | 6.2 | −1.0 | 1.0 | 1.4 | 1.1 | −1.0 | NA |
| HPRT1 | NM_000194 | −2.1 | −2.0 | −1.4 | −1.3 | 1.2 | 1.3 | 1.0 | 1.2 | −1.7 | 1.4 |
| HSPA1A | NM_005345 | 2.3 | 4.4 | −1.0 | 1.1 | −1.0 | −1.3 | 1.7 | 1.5 | 1.6 | 2.1 |
| HSPA1B | NM_005346 | 4.4 | 5.2 | 1.2 | 1.3 | −1.0 | −1.3 | 1.7 | 1.5 | 1.6 | 3.5 |
| HSPA1L | NM_005527 | 1.0 | 1.5 | 1.5 | 1.9 | 1.0 | −1.1 | 1.0 | 1.0 | 1.3 | −3.9 |
| HSPA6 | NM_002155 | 24.1 | 56.4 | 1.4 | 6.4 | −1.1 | −1.8 | 1.2 | 1.0 | 2.0 | −3.9 |
| IFI44 | NM_006417 | −1.3 | 1.1 | −2.0 | −1.4 | −1.6 | 1.0 | 1.0 | −1.6 | 1.1 | 2.7 |
| IGF1 | NM_000618 | −1.6 | −1.0 | −1.4 | −1.3 | 1.1 | 1.1 | −1.1 | 1.2 | −1.0 | −1.2 |
| IGF2 | NM_000612 | −1.4 | 1.9 | −2.0 | −1.3 | 1.0 | 1.0 | −1.2 | 1.0 | 1.3 | 1.1 |
| IL10 | NM_000572 | 3.0 | 2.2 | 1.1 | 1.2 | 1.1 | 1.1 | 1.2 | 1.0 | −1.5 | −2.8 |
| IL11 | NM_000641 | 3.3 | 3.1 | 4.2 | 5.2 | −1.1 | 1.1 | 1.2 | 1.4 | −1.2 | NA |
| IL1A | NM_000575 | 1.1 | −1.1 | 1.1 | 1.1 | −1.0 | 1.0 | 1.1 | 1.0 | −1.0 | −5.3 |
| IL33 | NM_033439 | 72.6 | 71.0 | 10.9 | 10.7 | −1.1 | 2.3 | −1.3 | −1.5 | 1.0 | NA |
| IL6 | NM_000600 | −1.2 | −1.0 | 2.6 | 2.8 | −1.0 | 1.5 | −1.3 | 1.0 | 1.0 | −1.5 |
| IL8 | NM_000584 | −2.0 | 21.7 | 14.8 | 15.9 | −1.2 | 1.0 | 1.0 | 1.0 | 1.1 | NA |
| IMMP1L | NM_144981 | 1.9 | 2.2 | −1.2 | 1.1 | −1.0 | 1.2 | 1.1 | 1.0 | −1.2 | NA |
| JUN | NM_002228 | 1.4 | 2.2 | 1.8 | 2.0 | 1.0 | −1.1 | −1.1 | −1.6 | 1.2 | NA |
| KIT | NM_000222 | −87.7 | −26.7 | −1.4 | −1.6 | −1.3 | 1.4 | −1.2 | −1.1 | −1.0 | NA |
| KL | NM_004795 | 1.7 | 2.4 | −1.4 | −1.3 | 1.0 | 1.0 | 1.1 | −1.1 | 2.4 | NA |
| KRAS | NM_004985 | −1.8 | −2.3 | 1.5 | −2.2 | −1.4 | 1.0 | 1.3 | 1.4 | 1.3 | 1.6 |
| MAPK14 | NM_001315 | −1.3 | −1.3 | 1.1 | 1.1 | 1.0 | −1.4 | −1.1 | 1.0 | −1.2 | 2.3 |
| MMP1 | NM_002421 | 3.1 | 3.5 | 1.4 | 1.5 | 1.0 | 1.0 | 1.0 | 1.0 | 1.1 | NA |
| NADSYN1 | NM_018161 | −1.4 | −1.4 | 1.3 | 1.2 | 1.2 | −1.1 | −1.3 | −1.1 | −1.5 | 3.4 |
| NEIL1 | NM_024608 | −2.3 | −2.8 | 1.5 | −1.9 | 1.5 | 1.0 | −1.1 | 1.2 | −1.1 | NA |
| NFKB1 | NM_003998 | −1.3 | −1.4 | 1.1 | −1.0 | −1.5 | −1.1 | 1.0 | −1.2 | 1.1 | 2.6 |
| NOS1 | NM_000620 | −3.7 | −2.5 | −3.6 | −4.0 | 1.9 | 1.2 | 1.1 | 1.1 | 1.2 | NA |
| NOS2 | NM_000625 | −1.6 | 1.2 | 1.1 | −1.1 | 1.1 | 1.0 | 1.1 | −1.1 | 1.0 | 2.6 |
| NOS3 | NM_000603 | 1.0 | 1.1 | −1.7 | −2.6 | 1.0 | −1.0 | 1.0 | 1.2 | −1.0 | −10.6 |
| PARP1 | NM_001618 | 1.1 | 1.1 | 1.8 | 1.7 | 1.3 | 1.0 | −1.0 | 1.3 | −1.1 | 4.4 |
| PARP2 | NM_005484 | 1.9 | 2.0 | −1.1 | −1.1 | 1.3 | 1.2 | 1.1 | 1.0 | −1.5 | −6.5 |
| PARP3 | NM_005485 | −1.7 | −1.6 | −1.3 | −1.2 | −1.0 | 1.1 | −1.2 | −1.1 | 1.1 | NA |
| PARP4 | NM_006437 | −2.1 | −1.7 | −1.1 | −1.0 | 1.1 | 4.7 | 1.0 | −1.2 | −1.1 | NA |
| PARP9 | NM_031458 | −3.0 | −2.4 | −1.1 | −1.1 | 1.0 | −1.1 | −2.6 | 1.1 | 1.0 | NA |
| PDGFRL | NM_006207 | −51.3 | −48.2 | −1.5 | −1.0 | −1.1 | 1.2 | −1.0 | 1.0 | 1.1 | NA |
| POT1 | NM_015450 | −1.3 | −1.1 | −1.0 | −1.0 | −1.0 | 1.2 | 1.0 | 1.0 | 1.1 | NA |
| PPARG | NM_015869 | −1.3 | −1.2 | −1.1 | −1.1 | −1.0 | −1.2 | 1.5 | −1.0 | −1.1 | −1.7 |
| PPARGC1 | NM_013261 | −1.3 | −1.2 | −1.1 | −1.1 | −1.0 | −1.2 | 1.5 | −1.0 | −1.1 | NA |
| PPC | SA_00103 | −1.4 | −2.1 | 2.8 | −1.6 | 1.0 | 1.0 | 1.1 | 1.0 | 1.0 | NA |
| PPC | SA_00103 | −1.4 | −2.1 | 2.8 | −1.6 | 1.0 | 1.0 | 1.1 | 1.0 | 1.0 | NA |
| PTGS2 | NM_000963 | 1.6 | 1.9 | 6.0 | 5.8 | −1.2 | 1.3 | 1.5 | −1.3 | 1.1 | −2.0 |
| RAP1A | NM_002884 | −1.4 | −1.5 | 1.1 | 1.1 | 1.3 | −1.2 | 1.2 | 1.1 | −1.3 | NA |
| RTC | SA_00104 | 1.0 | −1.1 | −1.1 | −1.3 | 1.1 | 1.2 | −2.4 | 1.0 | −1.1 | NA |
| RTC | SA_00104 | 1.0 | −1.1 | −1.1 | −1.3 | 1.1 | 1.2 | −2.4 | 1.0 | −1.1 | NA |
| S100A7 | NM_002963 | 9.4 | 13.0 | 2.4 | 3.1 | 1.1 | 1.3 | −1.2 | 1.0 | −1.0 | NA |
| SERPINB | NM_002575 | 1.1 | 1.1 | 1.0 | −1.0 | 1.1 | 1.0 | 1.0 | −2.0 | 1.0 | NA |
| SHC1 | NM_003029 | −1.1 | 1.3 | 1.3 | 1.4 | −1.4 | −1.1 | −1.5 | −1.0 | 1.6 | 1.0 |
| SIRT1 | NM_012238 | 1.4 | 1.3 | −1.0 | 1.1 | −1.2 | 1.1 | 1.4 | 1.0 | 1.1 | 1.3 |
| SIRT2 | NM_012237 | −1.2 | −1.0 | 1.1 | 1.1 | −1.0 | −1.1 | −1.3 | 1.0 | −1.1 | NA |
| SIRT3 | NM_012239 | −3.4 | −1.2 | −1.1 | −1.2 | −1.1 | −1.2 | −1.8 | −1.5 | −1.1 | NA |
| SIRT4 | NM_012240 | −2.1 | −1.8 | −3.8 | −2.3 | 1.1 | 1.0 | 1.1 | −1.2 | −1.1 | NA |
| SOD1 | NM_000454 | −1.0 | −1.0 | −1.2 | −1.1 | −1.0 | −1.1 | −1.1 | −1.0 | −1.3 | 1.2 |
| SOD2 | NM_000636 | 2.7 | 2.3 | 1.6 | 1.5 | −1.2 | 1.0 | −1.1 | 1.5 | 1.1 | 1.3 |
| TEP1 | NM_007110 | −1.9 | −1.7 | −1.3 | 1.0 | 1.1 | 1.1 | 1.5 | 1.2 | 1.1 | 1.1 |
| TERF2 | NM_005652 | −1.1 | 1.6 | 1.3 | 1.3 | 1.0 | −1.1 | 1.2 | −1.5 | 1.1 | NA |
| TERT | NM_198255 | −1.0 | 1.4 | 1.0 | −1.1 | 1.1 | −1.3 | −1.0 | 1.3 | 1.0 | 1.2 |
| TGFB1 | NM_000660 | 1.0 | 1.9 | 1.3 | 1.2 | −1.1 | −1.2 | 1.1 | 1.0 | 1.0 | NA |
| TIMM22 | NM_013337 | 1.3 | 1.3 | 1.0 | −1.0 | 1.1 | −1.1 | 1.3 | 1.4 | 1.0 | NA |
| TIMP3 | NM_000362 | 1.2 | 1.6 | 1.8 | 1.1 | 1.2 | −1.3 | 1.2 | 1.0 | 1.4 | NA |
| TINF2 | NM_012461 | −1.0 | −1.1 | −1.2 | −1.1 | 1.1 | 1.1 | −1.4 | −1.4 | −1.0 | NA |
| TNF | NM_000594 | −1.7 | −1.7 | 1.0 | 1.3 | −1.1 | −2.6 | 1.2 | −1.1 | −1.2 | −18.4 |
| TOMM40 | NM_006114 | 2.4 | 2.2 | 1.6 | 1.9 | 1.0 | −1.8 | 1.2 | 1.0 | 1.3 | NA |
| TP53 | NM_000546 | −3.1 | −2.0 | −1.5 | −1.5 | −1.1 | −1.3 | −1.0 | 1.2 | 1.2 | −1.2 |
| TPP1 | NM_000391 | −1.5 | −1.3 | 1.0 | −1.0 | −1.1 | 1.0 | 1.1 | −1.3 | 1.1 | NA |
| UBE2S | NM_014501 | 1.1 | −2.0 | −1.4 | −1.1 | 1.2 | −1.3 | 1.0 | 1.0 | 1.0 | NA |
| VEGFA | NM_003376 | 1.1 | 1.9 | 1.3 | 1.6 | −1.7 | −1.3 | 1.2 | −1.1 | 1.2 | NA |

A: Coffee cherry + H2O2 Expression (24 hours)
B: H2O2 Alone Expression (24 hours)
C: Coffee cherry + H2O2 Expression (8 hours)
D: H2O2 Alone Expression (8 hours)
E: Idebenone Alone Expression
F: Idebenone + UVB Expression
G: CoffeBerry Alone Expression
H: CoffeeBerry + UVB Expression
I: UVB Alone Expression
J: Green Tea + UVB Expression

DATA TABLE 12
Anti Aging Genes

| Official Gene | | | | | | | | | | | | | |
|---|---|---|---|---|---|---|---|---|---|---|---|---|---|
| Symbol | GenBank | A | B | C | D | E | F | G | H | I | J | K | L |
| ADAM1 | Y09232.1 | −1.4 | −1.1 | 1.1 | −1.1 | −0.3 | 2.2 | | | | | | |
| ANXA1 | NM_000700.1 | 3.2 | 2.4 | 1.1 | 1.5 | 0.7 | −0.4 | −1.2 | −1.1 | 1.0 | 1.0 | −1.0 | |
| APBB1 | L77864.1 | 1.4 | 1.4 | 1.6 | 1.6 | 0.1 | 0.0 | −1.0 | −1.4 | 1.3 | −1.3 | −1.1 | |
| APOB | NM_000384.1 | −1.3 | 1.4 | −1.0 | −1.2 | −2.7 | 0.2 | 1.0 | 1.0 | 1.0 | −1.1 | 1.1 | |
| APOE | M12529.1 | 1.2 | 1.4 | −1.2 | −1.7 | −0.2 | 0.5 | 1.2 | −1.2 | 1.0 | 1.2 | 1.1 | 1.0 |
| AREG | NM_001657.1 | 4.6 | 4.2 | −1.3 | 1.4 | 0.4 | −2.7 | 1.0 | 1.0 | 1.0 | 1.4 | −1.1 | |
| ATM | U26455.1 | 1.1 | −1.2 | 1.5 | 1.3 | 2.3 | 0.3 | −1.6 | 1.0 | 1.0 | −1.1 | 1.0 | |
| BAT1 | Z37166.1 | −1.0 | 1.0 | 1.0 | 1.1 | −2.1 | −0.1 | 1.1 | −1.2 | 1.3 | 1.0 | 1.1 | |
| BAX | NM_004324.1 | −1.5 | −1.3 | −1.0 | 1.0 | −0.2 | −2.0 | −1.1 | −1.5 | 1.3 | 1.0 | 1.1 | 1.4 |
| BCL2 | NM_000633.1 | −1.1 | 1.0 | 1.8 | 1.8 | −2.2 | 0.0 | −1.1 | 1.2 | −1.2 | −1.1 | 1.0 | 4.2 |
| BCL2L1 | NM_001191.1 | 1.2 | 1.2 | 1.1 | −1.2 | 0.0 | 2.3 | −1.3 | 1.0 | 1.0 | −1.1 | −1.1 | |
| BMP2 | NM_001200.1 | 10.3 | 8.6 | 8.4 | 10.5 | 1.7 | −2.1 | 1.3 | −1.7 | −1.1 | 1.2 | 1.1 | |
| BRCA2 | NM_000059.1 | 1.7 | 1.5 | 1.3 | 1.4 | 0.3 | −0.1 | 1.1 | −1.1 | 1.0 | −1.1 | 1.1 | |
| CANX | NM_001746.1 | 1.5 | 1.8 | 1.2 | 1.1 | −0.3 | 0.1 | −1.0 | 1.0 | 1.3 | −1.1 | −1.0 | |
| CASP7 | NM_001227.2 | −1.2 | −1.1 | −1.1 | −1.1 | −0.1 | 0.0 | 1.1 | 1.2 | 1.0 | −1.0 | −1.1 | |
| CASP8 | X98172.1 | 1.2 | 1.0 | −1.3 | −1.7 | 0.1 | 0.4 | −1.0 | 1.1 | −1.1 | −1.8 | 1.1 | |
| CCL5 | NM_002985.1 | 1.4 | 1.6 | −1.2 | 1.1 | −0.1 | −2.3 | 1.4 | −2.1 | −1.0 | 1.3 | 1.3 | |
| CCNA1 | NM_003914.2 | 1.2 | 1.2 | −1.0 | 1.2 | 0.0 | −2.3 | 1.0 | 1.0 | −1.1 | −1.3 | 1.2 | |
| CCNB1 | NM_031966.1 | 2.5 | 2.8 | −1.8 | −1.7 | −0.3 | −0.1 | 1.3 | −1.1 | −1.3 | 1.0 | 1.2 | |
| RPL13A | X56932.1 | −1.0 | −1.0 | −1.0 | −1.0 | 0.0 | 0.0 | 1.0 | −1.1 | 1.0 | −1.4 | −1.1 | |
| CCND1 | NM_053056.1 | 1.5 | 1.4 | 2.2 | 2.1 | 0.1 | 0.0 | −2.1 | −1.4 | 1.0 | 1.7 | 1.2 | |
| CCND2 | NM_001759.2 | 1.3 | 1.1 | −1.0 | −1.2 | 0.2 | 0.2 | −2.3 | 1.5 | 1.0 | 1.0 | −1.4 | |
| CCND3 | NM_001760.2 | 1.0 | −1.1 | 1.2 | 1.5 | 2.1 | −0.3 | 1.0 | −1.2 | −1.1 | 1.2 | −1.1 | |
| CCNE1 | NM_001238.1 | −2.3 | 1.1 | 1.1 | 1.6 | −3.4 | −0.5 | −1.0 | 1.2 | 1.4 | −1.0 | −1.2 | |
| CCNF | NM_001761.1 | 3.0 | 1.6 | 1.3 | 1.7 | 1.4 | −0.4 | 1.0 | −1.6 | 1.2 | 1.1 | −1.2 | |
| CCNG1 | U53328.1 | −2.2 | −2.1 | −1.4 | −1.6 | −0.1 | 0.1 | −1.1 | −1.2 | 1.5 | 1.0 | −1.1 | |
| CCNH | NM_001239.2 | −1.5 | 1.0 | 1.3 | 1.5 | −2.5 | −0.3 | 1.0 | 1.2 | −1.6 | −1.4 | −1.0 | |
| CDC42 | NM_001791.2 | −1.1 | 1.0 | −1.2 | −1.1 | −2.2 | 0.0 | 1.1 | 1.5 | −1.3 | 1.1 | 1.3 | |
| CDK2 | NM_001798.2 | 2.2 | 2.4 | 1.7 | 1.6 | −0.2 | 0.2 | 1.4 | −1.4 | 1.3 | 1.0 | −1.0 | |
| CDK4 | U79269.1 | 1.1 | 1.1 | −1.1 | −1.1 | 0.0 | 0.0 | 1.0 | −1.0 | 1.1 | −1.1 | −1.0 | |
| CDK5R1 | NM_003885.1 | 2.2 | 2.3 | −1.0 | −2.2 | −0.1 | 1.1 | −1.2 | −1.1 | 1.1 | 1.4 | 1.1 | |
| CDKN1A | U03106.1 | 1.7 | 1.0 | 1.1 | 1.2 | 0.7 | −0.1 | −1.7 | −1.3 | 1.1 | 1.2 | −1.7 | |
| CDKN1B | NM_004064.2 | −1.4 | −1.3 | −1.8 | −1.7 | 0.0 | −0.2 | 1.1 | 1.3 | 1.1 | 1.1 | 1.3 | |
| CDKN1C | NM_000076.1 | −1.2 | −1.5 | −2.2 | −2.6 | 0.3 | 0.4 | 1.0 | 1.4 | 1.2 | −1.0 | 2.2 | |
| CDKN2A | L27211.1 | −1.1 | −1.1 | −1.1 | −1.1 | −0.1 | 0.0 | −1.0 | −1.0 | −1.6 | 1.0 | 1.1 | |
| CENPA | U14518.1 | 2.4 | 3.0 | −2.2 | −1.8 | −0.5 | −0.3 | 1.5 | −1.2 | 1.0 | −1.2 | −1.0 | |
| CENPF | U30872.1 | 1.7 | 1.3 | −3.1 | −2.2 | 0.4 | −0.9 | 1.1 | −1.2 | −1.2 | −1.2 | 1.1 | |
| CKB | M16364.1 | 1.1 | 1.0 | 1.1 | −1.0 | 0.1 | 2.1 | −1.3 | −1.1 | 1.1 | 1.0 | 1.2 | |
| ALDOA | NM_000034.1 | 1.4 | 1.3 | 1.4 | 1.2 | 0.1 | 0.1 | −1.2 | 1.1 | 1.1 | −1.2 | 1.1 | |
| CLU | J02908.1 | −1.1 | 1.0 | 1.9 | 2.6 | −2.1 | −0.8 | 1.2 | −1.3 | 1.0 | 1.1 | 1.2 | |
| COL15A1 | NM_001855.1 | −2.9 | −2.0 | 1.1 | 1.3 | −0.9 | −0.1 | −1.9 | 1.2 | −1.1 | 1.6 | 1.0 | |
| COL1A1 | NM_000088.2 | −1.1 | −1.1 | 1.0 | 1.0 | 0.0 | 0.0 | −1.1 | −1.0 | 1.4 | 1.0 | −1.8 | |
| COL3A1 | NM_000090.2 | −4.4 | −5.3 | −4.8 | −5.7 | 0.9 | 1.0 | 1.2 | −1.7 | 1.2 | −2.0 | −1.0 | |
| K-ALPHA-1 | NM_006082.1 | 1.8 | 1.6 | 1.3 | 1.2 | 0.2 | 0.1 | | | | | | |
| COL6A2 | NM_058175.1 | −1.1 | −1.1 | 1.1 | 1.1 | 0.0 | −0.1 | −1.1 | −1.2 | −1.2 | −1.1 | −1.1 | |
| CSF2 | M11220.1 | 1.1 | 1.1 | 1.1 | 1.2 | 0.0 | −0.1 | 1.0 | 1.0 | −1.1 | −1.0 | 1.2 | |
| CST6 | U62800.1 | 1.5 | 1.7 | −1.0 | −1.0 | −0.1 | 0.0 | −1.6 | 1.2 | 1.0 | −1.2 | 1.2 | |
| CTGF | U14750.1 | 1.4 | 1.3 | 2.7 | 2.3 | 0.0 | 0.3 | −1.2 | 1.4 | 1.4 | 1.1 | −1.0 | |
| CTSD | M11233.1 | 4.2 | 4.5 | 1.4 | 2.1 | −0.3 | −0.7 | −1.1 | −1.2 | 1.4 | 1.3 | 2.0 | |
| CTSH | NM_004390.1 | −1.3 | −1.3 | −1.2 | −1.4 | 0.0 | 0.2 | 1.2 | 1.2 | −1.3 | 1.4 | −1.2 | |
| CTSS | M90696.1 | 1.2 | 1.1 | −1.0 | −1.0 | 0.2 | 0.0 | −1.9 | 1.0 | −1.4 | −1.5 | −1.1 | |
| CTSZ | AF136273.1 | −1.3 | −1.0 | 1.4 | 1.4 | −0.3 | 0.0 | −1.0 | −1.1 | 1.6 | 1.2 | −1.0 | |
| DDIT3 | S40706.1 | 1.3 | 1.3 | −1.6 | −1.3 | 0.0 | −0.3 | −1.4 | 1.1 | 1.2 | 1.3 | −1.0 | |
| DHFR | NM_000791.2 | −2.2 | −1.6 | −1.4 | −1.1 | −0.6 | −0.3 | 1.2 | 1.0 | 1.3 | 1.0 | 1.2 | |
| DNAJB1 | D49547.1 | 1.4 | 1.6 | −1.0 | −1.1 | −0.2 | 0.0 | −1.1 | −1.1 | 3.1 | 1.6 | 1.4 | |
| DPT | XM_001897.8 | −3.4 | −2.6 | −1.9 | −1.7 | −0.8 | −0.2 | 1.4 | 1.1 | 1.0 | 1.3 | 1.1 | |
| DSG1 | AF097935.1 | −1.1 | 1.8 | −1.3 | −1.5 | −2.9 | 0.2 | 1.0 | 1.4 | −1.3 | −1.0 | −1.1 | |
| E2F1 | NM_005225.1 | 2.6 | 2.4 | 2.4 | 2.4 | 0.2 | 0.0 | 1.4 | 1.1 | 1.1 | 1.1 | 1.1 | |
| E2F5 | U31556.1 | 1.1 | −1.1 | 1.3 | 1.1 | 2.3 | 0.2 | −1.4 | 1.1 | −1.1 | 1.0 | −1.0 | |
| EEF1A1 | AY043301.1 | −1.5 | −1.4 | 1.1 | −1.0 | −0.1 | 2.2 | −1.0 | −1.2 | 1.0 | −1.3 | −1.1 | |
| EGR1 | NM_001964.1 | 1.3 | 1.2 | −1.3 | −1.6 | 0.1 | 0.2 | −1.2 | 1.4 | 1.0 | 1.0 | −1.3 | |
| ACTB | NM_001101.2 | 1.1 | 1.1 | 1.1 | 1.0 | 0.0 | 0.0 | | −1.1 | −1.2 | 1.4 | −1.1 | |
| EGR2 | NM_000399.2 | −6.6 | −2.5 | −1.8 | 1.0 | −4.1 | −2.9 | −1.6 | 1.7 | −1.3 | −1.9 | −1.6 | |
| EGR3 | NM_004430.1 | −1.6 | −1.4 | 1.9 | 1.5 | −0.2 | 0.3 | −2.7 | 1.4 | 1.1 | 1.2 | 1.1 | |
| EIF3S6 | NM_001568.1 | −1.3 | −1.3 | 1.2 | 1.2 | 0.0 | 0.0 | | | | | | |
| EIF4A1 | NM_001416.1 | 1.1 | 1.1 | 1.1 | 1.1 | 0.0 | 0.0 | 1.0 | −1.1 | −1.1 | 1.0 | 1.0 | |
| ELN | NM_000501.1 | 1.5 | 1.6 | 1.5 | 1.5 | −0.1 | 0.0 | 1.0 | 1.0 | 1.3 | 1.3 | −1.0 | |
| EPC2 | AF286904.1 | −1.1 | −1.1 | −1.2 | −1.1 | 0.0 | −0.1 | 1.0 | 1.3 | 1.0 | 1.3 | −1.5 | |
| ETFB | NM_001985.1 | −1.2 | −1.6 | −1.0 | −1.1 | 0.4 | 0.0 | 1.0 | −1.2 | −1.1 | 1.0 | 1.4 | |
| EWSR1 | NM_005243.1 | 1.5 | 1.5 | 1.7 | 1.4 | 0.0 | 0.4 | 1.1 | −1.4 | 1.5 | 1.2 | 2.8 | |
| FES | X52192.1 | −2.3 | −2.6 | −1.7 | −1.3 | 0.3 | −0.4 | 1.3 | −1.4 | 1.2 | 1.0 | 1.0 | |
| FLG | M60502.1 | 1.0 | 1.1 | −1.3 | −1.1 | −0.1 | −0.2 | −1.9 | 1.0 | −1.4 | 1.0 | 1.6 | |
| FLT1 | NM_002019.2 | 2.5 | 1.0 | −1.6 | −2.0 | 1.5 | 0.4 | 1.1 | 1.0 | −1.2 | −1.2 | −1.2 | |
| FMOD | NM_002023.2 | 1.1 | 1.2 | 1.3 | 1.2 | 0.0 | 0.1 | 1.2 | 1.1 | 1.1 | 1.0 | 1.3 | |
| FN1 | X02761.1 | −2.4 | −1.7 | 1.0 | −1.2 | −0.7 | 2.2 | −1.5 | −1.2 | −1.2 | −1.3 | −1.2 | |
| FOS | NM_005252.2 | 1.4 | 1.3 | 1.1 | −1.0 | 0.1 | 2.1 | 3.5 | −1.4 | 1.4 | 1.2 | −1.1 | |
| G6PD | NM_000402.1 | −1.1 | −1.1 | −1.0 | −1.2 | 0.0 | 0.2 | −1.2 | −1.2 | 1.3 | −1.7 | −1.2 | |
| GAA | NM_000152.2 | −1.3 | −1.3 | 1.4 | 1.5 | 0.0 | 0.0 | 1.0 | −1.1 | 1.1 | 1.0 | 1.0 | |
| GLB1 | M34423.1 | −1.2 | −1.4 | 1.0 | 1.1 | 0.2 | 0.0 | −1.0 | 1.0 | −1.0 | 1.0 | −1.0 | |
| GPX1 | M21304.1 | −1.3 | −1.3 | −1.1 | −1.1 | 0.0 | 0.0 | −1.2 | −1.1 | −1.1 | 1.1 | 2.0 | |
| GRB2 | NM_002086.1 | 1.1 | −1.1 | −1.1 | −1.1 | 2.2 | 0.0 | −1.1 | −1.2 | −1.1 | −1.0 | −1.0 | |
| GSTP1 | NM_000852.2 | 1.1 | 1.0 | −1.1 | −1.0 | 0.1 | −0.1 | 1.1 | −1.1 | 1.0 | 1.0 | 1.0 | |
| GSTT1 | NM_000853.1 | −1.0 | 1.3 | 1.1 | −1.2 | −2.3 | 2.3 | −1.2 | 1.1 | −1.1 | −1.1 | −1.0 | |
| HBEGF | M60278.1 | −1.1 | −1.1 | 1.1 | −1.1 | 0.0 | 2.1 | −1.5 | 1.8 | 1.2 | 1.0 | 1.0 | |
| HIST1H2BK | NM_080593.1 | 2.9 | 2.4 | 1.1 | 1.1 | 0.5 | 0.0 | −1.5 | 1.0 | 2.2 | 1.2 | 1.0 | |
| HIST1H3I | NM_003533.2 | 2.4 | 3.6 | 1.4 | 1.7 | −1.2 | −0.3 | 1.1 | −1.1 | −1.5 | −1.3 | 1.3 | |
| HIST1H4I | NM_003495.2 | −1.4 | −1.4 | −1.4 | −1.1 | 0.0 | −0.3 | −1.1 | 1.1 | 2.6 | −1.1 | 1.0 | |
| HLF | M95585.1 | −2.4 | 1.0 | −1.1 | 1.1 | −3.5 | −2.2 | 1.0 | 1.0 | 1.0 | −1.5 | 1.1 | |
| HMOX1 | NM_002133.1 | 34.2 | 22.3 | 6.2 | 6.2 | 11.9 | 0.0 | 1.1 | 1.0 | 1.4 | 1.1 | −1.0 | |
| HSPA4 | AB023420.1 | 1.1 | −1.1 | −1.8 | −1.6 | 2.2 | −0.3 | −1.0 | −1.2 | −1.0 | −1.2 | −1.1 | |
| HSPA6 | NM_002155.1 | 24.1 | 56.4 | 1.4 | 6.4 | −32.3 | −5.0 | 1.4 | −1.8 | 1.2 | 1.0 | 2.0 | |
| PPIE | AF042385.1 | −2.3 | −2.1 | −1.5 | −1.0 | −0.2 | −0.5 | −1.1 | −1.2 | −1.4 | −1.0 | −1.7 | |
| HSPB1 | AB020027.1 | −1.2 | −1.2 | −1.3 | −1.3 | 0.0 | 0.0 | 1.0 | −1.1 | −1.3 | 1.0 | 1.0 | |
| HSP90AA1 | X15183.1 | 1.1 | 1.2 | 1.1 | 1.2 | −0.1 | 0.0 | 1.2 | −1.4 | 1.3 | 1.0 | −1.0 | |
| KRT2B | M99063.1 | −1.6 | 1.7 | 1.6 | 1.9 | −3.3 | −0.3 | | | | | | |
| ICAM1 | J03132.1 | 1.6 | 1.6 | 2.3 | 2.3 | 0.0 | 0.0 | 1.0 | 1.1 | 1.1 | −1.2 | −1.0 | |
| ID1 | X77956.1 | 9.5 | 8.2 | −1.0 | −1.1 | 1.2 | 0.1 | 1.2 | −1.0 | −1.6 | 1.1 | −1.5 | |
| ID2 | M97796.1 | 1.2 | 1.2 | −1.1 | −1.0 | 0.0 | −0.1 | 1.2 | 1.2 | 1.0 | 1.0 | 1.0 | |
| IFNG | X13274.1 | 1.2 | 1.0 | 1.3 | −1.2 | 0.2 | 2.5 | −1.0 | −1.1 | 1.0 | −1.2 | 2.3 | |
| IGF1 | X57025.1 | −1.0 | −1.0 | −1.4 | −1.3 | 0.0 | 0.0 | 1.0 | 1.1 | −1.1 | 1.2 | 1.2 | −1.8 |
| IGF1R | NM_000875.2 | −2.7 | −2.6 | −1.5 | −1.5 | −0.2 | 0.0 | −1.2 | −1.2 | −1.5 | 1.2 | 1.0 | |
| IGFBP2 | M35410.1 | −2.1 | 1.0 | 1.2 | 1.2 | −3.1 | −0.1 | −1.2 | 1.2 | 1.0 | 1.4 | 1.1 | |
| IGFBP3 | X64875.1 | −1.0 | −1.1 | 1.1 | 1.1 | 0.0 | 0.0 | −1.3 | −1.1 | 1.2 | −1.6 | −1.3 | |
| IGFBP5 | M65062.1 | −3.9 | −3.3 | 1.1 | 1.3 | −0.6 | −0.2 | −1.4 | 1.2 | 1.1 | 1.4 | 1.2 | |
| IL10 | NM_000572.1 | 3.0 | 2.2 | 1.1 | 1.2 | 0.8 | 0.0 | −1.0 | 1.1 | 1.2 | 1.0 | −1.5 | −2.1 |
| GAPDH | NM_002046.2 | 1.0 | 1.0 | −1.0 | −1.0 | 0.0 | 0.0 | −1.1 | −1.4 | −1.5 | 1.1 | 1.1 | |
| IL11 | NM_000641.1 | 3.3 | 3.1 | 4.2 | 5.2 | 0.2 | −1.0 | −1.0 | 1.1 | 1.2 | 1.4 | −1.1 | |
| IL11RA | NM_004512.1 | −1.0 | 1.1 | −1.1 | −1.0 | −2.2 | 0.0 | 1.1 | −1.2 | 1.0 | −1.0 | −1.1 | |
| IL12A | M65291.1 | 1.8 | 1.7 | 1.6 | 2.1 | 0.1 | −0.5 | −2.1 | −1.1 | −1.6 | 1.2 | 1.1 | |
| IL15 | NM_000585.1 | −1.4 | −1.3 | −1.6 | −1.4 | −0.1 | −0.2 | −1.0 | 1.2 | −1.0 | 2.3 | 1.1 | |
| IL1A | NM_000575.1 | 1.1 | −1.1 | 1.1 | 1.1 | 2.2 | −0.1 | −1.0 | 1.0 | 1.1 | 1.0 | −1.0 | −3.0 |
| IL1B | M15330.1 | 1.5 | 1.6 | −1.6 | −1.1 | −0.1 | −0.5 | −2.1 | 1.3 | −1.1 | 1.0 | 1.1 | |
| IL2 | U25676.1 | −1.3 | −1.4 | 1.1 | −1.3 | 0.2 | 2.4 | 1.0 | 1.0 | −1.3 | 1.5 | 1.0 | |
| IL3 | M20137.1 | 1.5 | 1.1 | 1.4 | −1.3 | 0.3 | 2.7 | 1.0 | 1.0 | −1.2 | 1.1 | −1.1 | |
| IL4 | NM_000589.1 | −2.8 | −1.2 | 1.3 | 1.1 | −1.6 | 0.2 | 1.1 | 1.4 | 1.3 | 1.0 | −1.1 | |
| IL6 | NM_000600.1 | −1.2 | −1.0 | 2.6 | 2.8 | −0.2 | −0.1 | −1.4 | 1.5 | −1.3 | 1.0 | 1.0 | 2.9 |
| IL8 | NM_000584.1 | 18.7 | 21.7 | 14.8 | 15.9 | −3.1 | −1.1 | 1.0 | 1.0 | 1.0 | 1.0 | 1.1 | |
| IVL | M13903.1 | 1.2 | 1.1 | 1.2 | 1.2 | 0.1 | −0.1 | 1.6 | −2.0 | −1.5 | −1.2 | 1.2 | |
| JUND | NM_005354.2 | −1.0 | −1.3 | −1.1 | −1.1 | 0.3 | 0.0 | 1.1 | 1.3 | −1.2 | −1.3 | 1.1 | |
| KIF23 | NM_004856.4 | −1.3 | 1.0 | −1.5 | −1.3 | −2.4 | −0.2 | 1.3 | −1.2 | 1.0 | −1.1 | 1.1 | |
| KIF2C | NM_006845.2 | 4.0 | 4.4 | −1.2 | −1.3 | −0.3 | 0.0 | 1.1 | −1.3 | 1.4 | 1.1 | 1.3 | |
| KRT1 | NM_006121.2 | 3.4 | 2.7 | −1.4 | 1.1 | 0.7 | −2.6 | 1.0 | 1.0 | −1.1 | −1.1 | −1.0 | |
| KRT10 | NM_000421.1 | 1.1 | 1.2 | 1.1 | 1.2 | −0.1 | −0.1 | 1.0 | 1.3 | −1.1 | −1.2 | −1.3 | |
| KRT14 | NM_000526.2 | 3.5 | 2.6 | 1.7 | 1.8 | 0.9 | −0.1 | −1.4 | −1.1 | 1.1 | 1.1 | 1.0 | |
| HK1 | M75126.1 | 1.6 | 1.3 | 1.7 | 1.5 | 0.3 | 0.2 | 1.0 | −1.1 | 1.0 | 1.4 | −1.1 | |
| KRT16 | AF061812.1 | 1.4 | 1.4 | −1.0 | −1.2 | 0.0 | 0.2 | −1.1 | −1.2 | 1.6 | −1.1 | 1.1 | |
| KRT17 | X62571.1 | 2.0 | 1.7 | 1.4 | 1.7 | 0.2 | −0.3 | −2.8 | −1.2 | 1.4 | 1.2 | −2.5 | |
| KRT19 | NM_002276.1 | 1.4 | 1.5 | −1.4 | −1.3 | −0.1 | −0.2 | −1.7 | −1.1 | 1.1 | 1.2 | 1.0 | |
| KRT6A | NM_005554.2 | −1.2 | −1.1 | −1.0 | 1.1 | −0.1 | −2.1 | 1.0 | −1.1 | 1.2 | 1.6 | −1.5 | |
| HPRT1 | NM_000194.1 | −2.1 | −2.0 | −1.4 | −1.3 | −0.1 | −0.2 | 1.1 | 1.3 | 1.0 | 1.2 | −1.7 | |
| LTA | NM_000595.2 | 1.4. | 1.8 | −2.2 | 1.0 | −0.4 | −3.2 | −1.1 | 2.0 | −1.1 | −1.2 | 1.4 | |
| MAP2K1 | NM_002755.2 | 1.5 | 1.4 | 1.4 | 1.3 | 0.1 | 0.0 | | −1.3 | 1.2 | 1.5 | −1.1 | |
| MAP2K2 | NM_030662.2 | −1.2 | −1.2 | −3.3 | −1.5 | 0.1 | −1.8 | | −1.3 | 1.0 | 1.3 | 1.0 | |
| MAP2K4 | NM_003010.1 | 1.1 | 1.1 | −1.1 | −1.1 | 0.1 | 0.0 | | 1.3 | 1.0 | 1.6 | −1.0 | |
| MAPK8 | L26318.1 | 1.6 | 1.7 | 1.3 | 1.5 | −0.2 | −0.2 | −1.3 | 1.1 | −1.1 | 1.2 | 1.2 | |
| MAPK9 | U09759.1 | −1.2 | −1.1 | −1.0 | 1.0 | 0.0 | −2.0 | −1.0 | −1.4 | 1.0 | 1.2 | 1.3 | |
| MAX | NM_002382.2 | 1.9 | 1.3 | 1.0 | −1.5 | 0.7 | 2.5 | −1.0 | −1.1 | −1.0 | 1.1 | 1.0 | |
| MCM2 | D21063.1 | 3.2 | 3.0 | 2.5 | 2.1 | 0.2 | 0.4 | 1.4 | −1.1 | −1.5 | 1.4 | 2.3 | |
| MDM2 | NM_002392.2 | 1.8 | 1.7 | 2.3 | 1.7 | 0.2 | 0.5 | −2.1 | −1.2 | 1.0 | 1.0 | −1.1 | |
| MKI67 | NM_002417.2 | 1.7 | 2.2 | −1.1 | −1.1 | −0.5 | 0.0 | 1.4 | −1.4 | 1.2 | 1.0 | −1.1 | |
| MMP1 | NM_002421.2 | 3.1 | 3.5 | 1.4 | 1.5 | −0.5 | −0.1 | −1.4 | −1.4 | −1.1 | −1.1 | −1.0 | |
| MMP10 | NM_002425.1 | 4.6 | 5.6 | 1.3 | 1.6 | −1.0 | −0.3 | 1.0 | 1.1 | 1.2 | −1.1 | −1.1 | |
| MMP11 | NM_005940.1 | 3.3 | 2.3 | −1.3 | 1.3 | 1.0 | −2.6 | 1.1 | −1.1 | 1.0 | 1.0 | −1.1 | |
| MMP12 | NM_002426.1 | 1.5 | 1.9 | −1.1 | 1.1 | −0.4 | −2.2 | 1.7 | 1.2 | −1.2 | −1.1 | −1.0 | |
| MMP13 | NM_002427.1 | −1.3 | −1.6 | −1.4 | −1.4 | 0.3 | 0.0 | 1.0 | 1.0 | 1.0 | −1.0 | −1.8 | |
| MMP14 | NM_004995.2 | 1.8 | 2.0 | 2.0 | 1.9 | −0.2 | 0.1 | −1.1 | −1.8 | 1.0 | 1.0 | 1.1 | |
| MMP15 | NM_002428.1 | −2.1 | −1.3 | −1.2 | −1.5 | −0.8 | 0.3 | −1.0 | −1.1 | −1.2 | −1.3 | 1.2 | |
| MDH1 | NM_005917.1 | −1.4 | −1.2 | −1.4 | −1.5 | −0.1 | 0.1 | 1.0 | 1.0 | 1.1 | 1.0 | 1.3 | |
| MMP2 | NM_004530.1 | 1.1 | 1.2 | 1.2 | 1.0 | −0.1 | 0.1 | −1.1 | −1.1 | 1.1 | −1.2 | 1.1 | |
| MMP3 | NM_002422.2 | 1.1 | 1.1 | 1.0 | 1.0 | 0.0 | 0.0 | 2.3 | −1.0 | 3.5 | 1.0 | −1.2 | |
| MMP7 | NM_002423.2 | −1.2 | −1.2 | −1.3 | −1.4 | 0.1 | 0.1 | −1.6 | 1.0 | −1.2 | 1.2 | 1.1 | |
| MSRA | AF183420.1 | −1.3 | 1.2 | 2.6 | 1.8 | −2.5 | 0.8 | −1.0 | −1.0 | 1.0 | 1.0 | 1.1 | |
| MT2A | V00594.1 | 1.7 | 1.7 | 1.1 | 1.1 | 0.0 | 0.0 | −1.3 | 1.1 | 1.1 | 1.2 | 1.3 | |
| MVK | M88468.1 | −1.0 | −1.2 | −1.3 | −1.5 | 0.2 | 0.2 | 1.6 | 1.1 | 1.4 | −1.5 | 1.3 | |
| MYBL2 | X13293.1 | 3.9 | 3.8 | 2.4 | 2.0 | 0.0 | 0.4 | 1.4 | −1.1 | −1.2 | 1.2 | 1.0 | |
| MYCBP | NM_012333.2 | 1.6 | 1.8 | 1.3 | 1.3 | −0.2 | 0.0 | −1.0 | −1.3 | −1.1 | 1.0 | −1.1 | |
| NCK1 | NM_006153.2 | 1.0 | −1.1 | −1.4 | −1.4 | 2.2 | 0.1 | 1.0 | 1.1 | 1.0 | −1.0 | −1.5 | |
| NF1 | NM_000267.1 | −3.0 | −1.7 | −1.1 | −1.1 | −1.3 | 0.1 | −1.1 | 1.2 | 1.2 | 1.1 | 1.0 | |
| NGFR | M14764.1 | 3.4 | 2.7 | 1.1 | 2.0 | 0.7 | −0.9 | 1.0 | | | | −1.0 | |
| NR1D1 | NM_021724.1 | 4.7 | 4.1 | 4.6 | 5.4 | 0.6 | −0.8 | −1.6 | 1.1 | 1.3 | −1.2 | 1.1 | |
| NRG1 | M94165.1 | 4.3 | 3.9 | 1.2 | −1.0 | 0.4 | 2.2 | 1.1 | 1.0 | −2.0 | −1.2 | −1.6 | |
| ODC1 | NM_002539.1 | 3.2 | 2.8 | 1.9 | 1.7 | 0.4 | 0.2 | 1.1 | 1.0 | −1.1 | 1.0 | 1.1 | |
| PAK1 | NM_002576.2 | −1.1 | 1.0 | 1.2 | 1.2 | −2.2 | 0.0 | −1.0 | −1.4 | −1.8 | −1.4 | 1.0 | |
| PARP1 | J03473.1 | 1.1 | 1.1 | 1.8 | 1.7 | 0.0 | 0.1 | 1.2 | 1.0 | −1.0 | 1.3 | −1.1 | 3.3 |
| PCNA | NM_002592.1 | 1.8 | 1.7 | 1.1 | 1.1 | 0.1 | −0.1 | −1.0 | 1.1 | 1.4 | 1.2 | −1.1 | |
| PDZK1IP1 | U21049.1 | −1.4 | 1.0 | −1.0 | −1.0 | −2.4 | 0.0 | 1.0 | 1.0 | −1.5 | −1.4 | 1.0 | |
| PITRM1 | AF061243.1 | −1.2 | 1.1 | −1.2 | −2.1 | −2.3 | 0.9 | −1.1 | −1.2 | 1.0 | 1.7 | 1.3 | |
| PKM2 | M26252.1 | −1.2 | −1.1 | 1.1 | 1.1 | −0.1 | 0.0 | −1.1 | −1.3 | −1.2 | −1.0 | 1.3 | |
| PLAT | NM_000930.2 | 1.7 | 1.6 | 3.7 | 3.4 | 0.1 | 0.3 | −1.1 | 1.1 | 1.0 | 1.0 | 1.2 | |
| PLAU | NM_002658.1 | 3.3 | 3.5 | 7.5 | 7.3 | −0.2 | 0.2 | −1.6 | 1.0 | −1.0 | −1.9 | 1.1 | |
| PLAUR | NM_002659.1 | 4.7 | 4.7 | 3.2 | 3.3 | 0.0 | −0.2 | −1.1 | −1.2 | −1.4 | −1.4 | −1.2 | |
| PLK1 | U01038.1 | −2.7 | −3.1 | −2.3 | −1.8 | 0.4 | −0.5 | 1.3 | −1.3 | 1.0 | 1.0 | 1.1 | |
| POLA2 | NM_002689.2 | 1.2 | 1.8 | 1.5 | 1.1 | −0.6 | 0.4 | 1.0 | −1.3 | 1.3 | −1.1 | 1.2 | |
| PRDX6 | NM_004905.1 | −1.7 | −2.4 | −1.1 | −1.5 | 0.6 | 0.4 | −1.5 | 1.1 | 1.0 | 1.0 | −1.1 | |
| HTRA1 | NM_002775.1 | −1.1 | −1.1 | 1.0 | 1.1 | −0.1 | 0.0 | 1.1 | 1.0 | −1.0 | 1.2 | −1.0 | |
| PSMA2 | NM_002787.1 | 1.1 | 1.1 | −1.1 | −1.0 | 0.0 | 0.0 | 1.1 | 1.0 | 1.2 | −1.2 | 1.0 | |
| PSMA3 | NM_002788.1 | −1.2 | −1.0 | 1.5 | 2.3 | −0.1 | −0.8 | 1.1 | 1.2 | 1.0 | 1.5 | −1.3 | |
| YWHAZ | M86400.1 | −1.2 | −1.1 | −1.2 | −1.2 | −0.1 | 0.0 | 1.0 | 1.1 | 1.0 | −1.0 | 1.2 | |
| PSMC6 | NM_002806.1 | 1.2 | 1.2 | −1.0 | −1.0 | 0.0 | 0.0 | 1.0 | 1.0 | 1.0 | 1.0 | 1.1 | |
| PSMD1 | NM_002807.1 | 1.4 | 1.3 | 1.1 | 1.1 | 0.1 | 0.0 | −1.0 | −1.3 | 1.2 | −1.1 | 1.1 | |
| PSMD11 | AB003102.1 | 1.5 | 1.4 | 1.1 | −1.0 | 0.1 | 2.1 | 1.1 | −1.4 | −1.1 | 1.2 | −1.1 | |
| PSMD12 | NM_002816.1 | −1.0 | 1.0 | −1.1 | −1.0 | −2.0 | −0.1 | 1.0 | 1.1 | −1.1 | −1.1 | −1.2 | |
| PTGS1 | NM_000962.2 | 1.1 | 1.2 | 1.5 | 1.5 | −0.1 | 0.0 | 1.2 | −1.3 | 1.0 | 1.0 | 1.1 | |
| PTGS2 | NM_000963.1 | 1.9 | 1.9 | 6.0 | 5.8 | 0.1 | 0.2 | −1.3 | 1.3 | 1.5 | −1.3 | 1.2 | |
| RAF1 | X03484.1 | 1.2 | −1.0 | 1.2 | 1.1 | 2.3 | 0.1 | −1.0 | −1.2 | 1.3 | −1.6 | 2.1 | |
| RB1 | NM_000321.1 | −1.1 | 1.0 | 1.5 | 1.6 | −2.2 | −0.1 | −1.5 | 1.2 | 1.2 | 1.3 | −1.9 | |
| RET | NM_000323.2 | 1.7 | 1.8 | −1.8 | 1.9 | −0.1 | −3.7 | −1.0 | 1.0 | 1.1 | 1.1 | 1.2 | |
| RORA | U04897.1 | −1.3 | −1.1 | 2.0 | 1.6 | −0.2 | 0.4 | −1.2 | −1.2 | −1.1 | 1.0 | 1.1 | |
| RPL3 | NM_000967.2 | −1.1 | −1.1 | −1.0 | −1.0 | 0.0 | 0.0 | 1.0 | −1.4 | −1.3 | −1.1 | −1.3 | |
| RPS10 | NM_001014.2 | 1.8 | 1.8 | 1.3 | 1.5 | 0.1 | −0.2 | 1.0 | 1.1 | 1.3 | −1.2 | −1.0 | |
| RPS9 | NM_001013.2 | 1.2 | 1.3 | 1.1 | −1.2 | −0.1 | 2.3 | 1.2 | −1.0 | 1.0 | 1.2 | −1.1 | |
| RRAS | NM_006270.2 | −1.2 | −1.3 | −1.3 | −1.2 | 0.1 | −0.1 | −1.1 | −1.1 | −1.1 | −1.0 | 1.0 | |
| RRM1 | NM_001033.1 | −1.5 | −1.1 | −2.7 | −2.4 | −0.4 | −0.2 | −1.0 | 1.0 | 1.0 | 1.0 | −1.1 | |
| S100A10 | M81457.1 | −3.7 | −4.6 | −5.4 | −4.9 | 0.9 | −0.5 | −1.7 | 1.0 | 1.1 | 1.2 | 1.3 | |
| S100A11 | D38583.1 | 1.1 | 1.0 | −1.1 | 1.0 | 0.0 | −2.1 | −1.1 | −1.1 | −1.5 | 1.0 | −1.4 | |
| S100A7 | M86757.1 | 9.4 | 13.0 | 2.4 | 3.1 | −3.5 | −0.8 | 2.7 | 1.3 | −1.2 | 1.0 | 1.0 | |
| S100A8 | NM_002964.2 | 1.4 | 1.3 | 1.1 | −1.3 | 0.1 | 2.4 | 1.0 | −1.1 | −1.5 | 1.0 | 1.2 | |
| SERPINB2 | J02685.1 | 24.8 | 25.0 | 9.2 | 9.0 | −0.2 | 0.2 | −2.9 | 1.0 | 1.1 | 1.0 | −1.0 | |
| SERPINE1 | M14083.1 | 1.0 | 1.2 | 1.7 | 1.6 | −0.2 | 0.0 | −1.4 | −1.2 | −1.1 | −1.2 | −2.2 | |
| SHC1 | U73377.1 | 1.2 | 1.3 | 1.3 | 1.4 | −0.1 | −0.1 | 1.0 | −1.2 | −1.4 | −1.0 | 1.6 | 1.3 |
| SLK | NM_014720.1 | −1.1 | −1.1 | −1.1 | 1.1 | 0.0 | −2.2 | −1.0 | 1.1 | 1.0 | −1.4 | 1.1 | |
| SMAD1 | U59423.1 | −1.2 | −1.2 | −1.4 | −1.3 | 0.0 | −0.1 | 1.2 | 1.6 | 1.0 | 1.0 | 1.2 | |
| SNCG | NM_003087.1 | 1.4 | 1.3 | −1.2 | −1.4 | 0.0 | 0.2 | 1.4 | 1.2 | −1.0 | 1.0 | 1.0 | |
| SOD2 | NM_000636.1 | 2.8 | 2.3 | 1.6 | 1.5 | 0.5 | 0.1 | 1.0 | 1.1 | −1.1 | −1.2 | 1.1 | −1.3 |
| SPARC | NM_003118.1 | −1.4 | −1.0 | 1.3 | 1.5 | −0.3 | −0.2 | −1.2 | 1.0 | 1.2 | 1.0 | 1.2 | |
| SDS | NM_006843.1 | 1.4 | 1.5 | −1.2 | 1.4 | −0.1 | −2.6 | −2.0 | 1.2 | 1.0 | 1.1 | 1.4 | |
| SPP1 | NM_000582.1 | 2.2 | 1.3 | 1.3 | 1.2 | 0.9 | 0.2 | −2.7 | 1.1 | 1.0 | −1.0 | 1.6 | |
| SPRR1B | NM_003125.1 | 1.0 | −1.2 | −1.1 | −1.3 | 2.2 | 0.2 | 1.0 | 1.0 | 1.0 | −1.5 | −1.2 | |
| SRI | NM_003130.1 | −1.1 | −1.0 | −1.1 | −1.0 | −0.1 | −0.1 | −1.0 | 1.0 | 1.0 | −1.3 | −1.0 | |
| STAT5A | L41142.1 | −1.3 | −1.6 | −1.0 | 1.1 | 0.3 | −2.1 | −1.0 | −1.2 | 1.6 | 1.0 | 1.1 | |
| STOML2 | AF282596. | 1.2 | 1.1 | 1.1 | −1.0 | 0.0 | 2.1 | 1.2 | −1.2 | −1.1 | 1.0 | −1.1 | |
| TAGLN | M95787.1 | 2.7 | 2.7 | 1.9 | 1.9 | 0.0 | 0.1 | −1.2 | −1.1 | −1.3 | 1.1 | 1.1 | |
| TBXA2R | D38081.1 | 1.1 | −1.1 | −1.1 | −1.1 | 2.2 | 0.0 | −1.1 | 1.2 | 1.2 | 1.1 | 1.1 | |
| TERC | U86046.1 | −3.3 | −2.8 | −1.5 | −1.6 | −0.4 | 0.1 | | | | | | |
| TERF1 | U40705.1 | −1.4 | −1.3 | −1.5 | −1.4 | −0.1 | −0.1 | −1.0 | 1.4 | −1.0 | −1.1 | 1.0 | |
| TERT | AF018167.1 | −1.0 | 1.4 | 1.0 | −1.1 | −2.5 | 2.1 | 1.1 | −1.3 | −1.0 | 1.3 | 1.0 | 1.2 |
| TFAP2A | M36711.1 | 1.8 | −1.7 | 3.0 | 4.2 | 3.5 | −1.2 | −1.2 | −1.4 | 1.0 | 1.1 | 1.6 | |
| TFAP2B | X95694.1 | −1.1 | 1.1 | 1.1 | 1.2 | −2.2 | −0.1 | 1.0 | 1.0 | 1.6 | 1.1 | 1.2 | |
| TFAP2C | NM_003222.2 | −2.1 | −1.9 | 1.0 | −1.3 | −0.2 | 2.3 | −1.1 | −1.3 | −1.2 | −1.2 | −1.4 | |
| TGFA | NM_003236.1 | 4.7 | 4.1 | 4.5 | 4.4 | 0.5 | 0.1 | 1.0 | −1.5 | 1.2 | 1.0 | −1.0 | |
| TGFB1 | NM_000660.1 | 2.0 | 1.9 | 1.3 | 1.2 | 0.2 | 0.1 | 1.0 | −1.2 | 1.1 | 1.0 | 1.0 | |
| TGFBR2 | D50683.1 | 1.2 | 1.1 | −1.0 | −1.2 | 0.1 | 0.2 | −1.3 | 1.0 | 1.0 | −1.6 | −1.3 | |
| TGM1 | NM_000359.1 | 5.2 | 1.2 | −1.3 | 1.9 | 4.1 | −3.2 | −1.1 | 1.0 | 1.0 | −1.0 | 1.1 | |
| TH | NM_000360.1 | 1.1 | 1.0 | 1.0 | 1.3 | 0.1 | −0.3 | 1.6 | −2.2 | −1.0 | 1.5 | 2.5 | |
| THBS1 | NM_003246.1 | −1.9 | −1.5 | 2.0 | 1.7 | −0.4 | 0.2 | −1.1 | −1.2 | 1.2 | 1.1 | 1.2 | |
| TIMP1 | NM_003254.1 | −1.0 | −1.0 | −1.0 | −1.0 | 0.0 | 0.0 | −1.4 | −1.3 | 1.0 | −1.2 | −1.4 | |
| TIMP2 | NM_003255.2 | −1.5 | −1.4 | −1.2 | −1.1 | −0.1 | 0.0 | −1.4 | 1.2 | 1.0 | 1.4 | 1.3 | |
| TK1 | NM_003258.1 | −1.0 | −1.2 | 1.4 | 1.5 | 0.2 | −0.1 | 1.6 | −1.2 | 1.4 | −1.2 | 1.0 | |
| TM4SF1 | M90657.1 | −1.1 | 1.1 | −1.6 | −1.3 | −2.2 | −0.3 | −1.1 | −1.5 | −1.4 | 1.0 | −1.0 | |
| TNF | NM_000594.1 | −1.7 | −1.7 | 1.0 | 1.3 | 0.0 | −0.3 | 1.1 | −1.4 | 1.1 | 1.0 | −1.2 | −6.8 |
| TNFRSF11B | NM_002546.2 | −3.8 | −3.0 | 1.3 | 1.3 | −0.8 | 0.0 | 1.0 | 1.0 | −1.2 | 1.1 | −1.4 | |
| TNFRSF1A | NM_001065.2 | 1.3 | 1.2 | −1.3 | −1.6 | 0.1 | 0.2 | −1.1 | −1.1 | 1.3 | 1.0 | 1.1 | |
| TFRC | NM_003234.1 | −2.0 | −2.0 | 1.6 | −1.8 | 0.1 | 3.4 | 1.1 | 1.0 | 1.2 | 1.0 | 1.1 | |
| TNFRSF1B | NM_001066.1 | 1.2 | 1.4 | 2.7 | 2.7 | −0.2 | 0.1 | −1.8 | −1.0 | −1.0 | 1.0 | 1.1 | |
| TOP2A | NM_001067.2 | −1.1 | 1.7 | −1.4 | −1.4 | −2.7 | −0.1 | 1.6 | −1.3 | −1.4 | −1.1 | 2.6 | |
| TP53 | AF307851.1 | −2.1 | −2.0 | −1.5 | −1.5 | −0.2 | 0.0 | 1.1 | −1.3 | −1.0 | 1.2 | 1.2 | −1.1 |
| TYMS | NM_001071.1 | −1.6 | −1.3 | −1.3 | −1.0 | −0.3 | −0.2 | 1.2 | 1.1 | 1.0 | 2.0 | 1.3 | |
| UBE2C | NM_007019.1 | 4.7 | 5.0 | −1.2 | −1.1 | −0.3 | −0.1 | 1.5 | −1.2 | 1.1 | 1.0 | −1.0 | |
| UBE2V1 | NM_003349.3 | −2.0 | −5.1 | −1.6 | −1.7 | 3.1 | 0.1 | −1.1 | −1.1 | −1.0 | 1.2 | 1.0 | |
| VEGFC | NM_005429.2 | 1.3 | 1.3 | 2.3 | 2.2 | 0.0 | 0.1 | 1.0 | −1.0 | −1.0 | −1.0 | 2.1 | |

A: Coffee cherry + H2O2 Expression (24 hours)
B: H2O2 Alone Expression (24 hours)
C: Coffee cherry + H2O2 Expression (8 hours)
D: H2O2 Alone Expression (8 hours)
E: Difference between Coffee cherry + H2o2 and H202 Alone Expression (24 hours)
F: Difference between Coffee cherry + H2o2 and H202 Alone Expression (8 hours)
G: Idebenone Alone Expression
H: Idebenone + UVB Expression
I: CoffeeBerry Alone Expression
J: CoffeeBerry + UVB Expression
K: UVB Alone Expression
L: Green Tea + UVB Expression

DATA TABLE 13
Inflammation Genes

| Official Gene | | | | | | | | | | | | | |
|---|---|---|---|---|---|---|---|---|---|---|---|---|---|
| Symbol | Genbank | A | B | C | D | E | F | G | H | I | J | K | L |
| ACTB | NM_001101.2 | −1.0 | −1.2 | 1.1 | −1.1 | 1.1 | | 1.1 | 1.1 | 1.1 | 1.0 | 0.0 | 0.0 |
| AGT | NM_000029.1 | 1.4 | 1.5 | 1.4 | 1.4 | 1.0 | | 1.1 | 1.0 | 1.2 | 1.1 | 0.1 | 0.1 |
| AGTR1 | M87290.1 | −1.0 | 1.0 | 1.0 | 1.1 | 1.0 | | 1.1 | 1.0 | −1.0 | −1.0 | 0.1 | 0.0 |
| ALDOA | NM_000034.1 | 1.2 | −1.3 | 1.2 | −1.2 | 1.1 | | 1.4 | 1.3 | 1.4 | 1.2 | 0.1 | 0.1 |
| ALOX15 | NM_001140.3 | 1.0 | 2.0 | −1.2 | 1.6 | 1.3 | | 1.7 | 2.3 | 1.9 | 1.1 | −0.6 | 0.8 |
| ALOX5 | NM_000698.1 | 1.1 | 1.0 | −1.6 | −1.2 | −1.0 | | 1.1 | −2.3 | 4.1 | 4.2 | 3.4 | 0.0 |
| AMH | NM_000479.2 | −1.0 | −1.1 | 1.2 | −1.1 | −1.1 | | 2.9 | 3.8 | 2.0 | 1.9 | −0.8 | 0.0 |
| AREG | NM_001657.1 | −1.2 | 1.0 | 1.0 | 1.4 | −1.1 | | 4.6 | 4.2 | −1.3 | 1.4 | 0.4 | −2.7 |
| ATF2 | NM_001880.2 | 1.0 | 1.5 | −1.3 | −1.3 | 1.2 | | −1.2 | −1.1 | −1.2 | −1.8 | −0.1 | 0.6 |
| B2M | NM_004048.2 | −1.0 | −1.2 | 1.1 | −1.6 | −1.8 | | 1.8 | 1.8 | 1.2 | 1.0 | 0.0 | 0.2 |
| BAD | NM_004322.2 | 1.0 | −1.1 | −1.2 | 1.0 | 1.2 | | 1.5 | 1.6 | 1.6 | 1.2 | −0.1 | 0.4 |
| BAG3 | NM_004281.2 | 1.0 | −1.1 | 1.0 | −1.5 | −1.4 | | 1.8 | 1.6 | 1.3 | 1.2 | 0.2 | 0.1 |
| BCL2 | NM_000633.1 | 1.0 | 1.2 | −1.2 | −1.1 | 1.0 | 3.5 | −1.1 | 1.0 | 1.8 | 1.8 | −2.2 | 0.0 |
| BCL3 | NM_005178.2 | 1.1 | 1.1 | 1.0 | 1.3 | −1.0 | | −1.1 | −1.0 | 1.0 | 1.2 | 0.0 | −0.1 |
| BDNF | M61176.1 | −1.0 | −1.1 | 1.2 | −1.0 | 1.1 | | −1.1 | −1.1 | 1.9 | 1.8 | 0.0 | 0.1 |
| BLR1 | X68149.1 | −1.2 | 1.2 | 1.6 | −1.5 | −1.1 | | 1.6 | 1.2 | −1.9 | −1.1 | 0.4 | −0.8 |
| BMP1 | M22488.1 | 1.4 | −1.3 | 1.0 | 1.4 | 1.3 | | −1.6 | −1.3 | 1.2 | 1.4 | −0.3 | −0.2 |
| BMP2 | NM_001200.1 | 1.1 | −1.7 | −1.1 | 1.2 | 1.0 | | 10.3 | 8.6 | 8.4 | 10.5 | 1.7 | −2.1 |
| BMP3 | NM_001201.1 | 1.0 | 1.0 | −1.0 | 1.1 | 1.2 | | 1.1 | −1.1 | −1.1 | 1.2 | 2.2 | −2.3 |
| BMP4 | M22490.1 | 1.0 | 1.3 | 1.2 | −1.1 | 1.5 | | 4.3 | 4.4 | −1.2 | −2.4 | −0.1 | 1.2 |
| BMP6 | NM_001718.2 | −1.3 | 1.0 | 1.0 | 1.0 | 1.1 | | 1.2 | −1.4 | −1.7 | −1.6 | 2.6 | −0.1 |
| BMP7 | NM_001719.1 | 1.1 | 1.8 | 1.0 | −1.1 | −2.2 | | 1.3 | 1.0 | −1.9 | −2.0 | 0.3 | 0.0 |
| BMPR2 | D50516.1 | −1.0 | −1.2 | 2.4 | −1.6 | 1.1 | | −2.2 | −1.9 | −1.4 | −1.3 | −0.3 | −0.1 |
| BSG | NM_001728.1 | 1.0 | −1.1 | 1.3 | −1.3 | −1.0 | | −1.4 | −1.3 | −1.3 | −1.3 | 0.0 | −0.1 |
| C3 | NM_000064.1 | −1.1 | −1.2 | 1.3 | −1.0 | 2.2 | | 3.0 | 3.0 | −1.1 | −1.1 | 0.0 | 0.0 |
| C5 | NM_001735.2 | | | | | | | −2.2 | −2.7 | −1.6 | −1.6 | 0.6 | 0.0 |
| CASP1 | M87507.1 | 1.0 | 1.0 | 1.5 | 1.0 | 1.2 | | −4.5 | −4.1 | −1.1 | −1.4 | −0.4 | 0.3 |
| CAV1 | NM_001753.3 | 1.0 | −1.5 | 1.2 | 1.0 | 1.2 | | −1.2 | −1.2 | −1.2 | −1.2 | 0.0 | 0.0 |
| CCL1 | NM_002981.1 | 1.1 | −1.1 | 1.1 | 1.0 | 1.1 | | −1.3 | −1.5 | −1.3 | 1.4 | 0.2 | −2.7 |
| CCL11 | NM_002986.2 | 1.0 | −1.6 | −1.2 | 1.1 | 1.0 | | 1.6 | 1.1 | 1.2 | 1.1 | 0.5 | 0.1 |
| CCL13 | NM_005408.2 | 1.1 | 1.0 | −1.2 | 1.1 | 1.0 | | −1.3 | −1.6 | 1.4 | 2.4 | 0.4 | −1.0 |
| CCL2 | NM_002982.3 | −1.1 | 1.1 | −1.2 | 1.0 | −1.1 | | 1.4 | 2.0 | 4.2 | 4.6 | −0.6 | −0.5 |
| CCL21 | NM_002989.2 | −1.2 | −1.3 | −1.0 | −1.1 | 1.0 | | 1.9 | 1.9 | 1.2 | 1.1 | 0.0 | 0.1 |
| CCL5 | NM_002985.1 | −1.0 | −2.1 | −1.0 | 1.3 | 1.3 | | 1.4 | 1.6 | −1.2 | 1.1 | −0.1 | −2.3 |
| CCL7 | NM_006273.2 | −1.0 | −1.0 | 1.5 | 1.0 | 1.1 | | −1.2 | −1.0 | 1.4 | 2.0 | −0.1 | −0.5 |
| CCL8 | NM_005623.2 | 1.1 | 1.0 | 1.0 | −1.6 | 1.1 | | −1.2 | −1.1 | 1.5 | 1.1 | −0.2 | 0.5 |
| CCR1 | NM_001295.2 | 1.1 | 1.0 | −1.8 | 1.0 | −8.2 | | 1.7 | 1.6 | 1.4 | 1.1 | 0.2 | 0.3 |
| CCR2 | NM_000647.3 | 1.2 | 1.0 | −1.2 | −1.1 | −1.0 | | 1.1 | −1.4 | 1.2 | 1.2 | 2.6 | 0.0 |
| CCR3 | NM_001837.2 | −1.3 | −1.1 | 1.1 | 1.0 | −1.1 | | 1.1 | 2.1 | −1.1 | −1.6 | −1.1 | 0.5 |
| CCR4 | NM_005508.4 | 1.2 | 1.8 | 1.3 | 1.1 | 1.1 | | −1.2 | −1.2 | −1.1 | −1.2 | 0.1 | 0.1 |
| CCR5 | NM_000579.1 | −1.1 | −1.1 | 1.1 | −1.1 | 1.1 | | −1.0 | 1.0 | −1.7 | 1.0 | −2.0 | −2.7 |
| CCR6 | U68030.1 | 1.0 | 1.0 | 1.1 | 2.0 | 2.3 | | −1.0 | 1.1 | 2.7 | 1.5 | −2.1 | 1.1 |
| CCR7 | NM_001838.2 | 1.0 | 1.5 | 1.3 | −1.3 | 1.2 | | −1.2 | −1.3 | 1.7 | 1.3 | 0.1 | 0.4 |
| CCR8 | NM_005201.2 | 1.0 | 1.0 | 1.2 | 1.2 | 1.6 | | −3.7 | −1.9 | −1.5 | −2.2 | −1.8 | 0.6 |
| CD2 | NM_001767.1 | −1.0 | 1.6 | 1.0 | −1.2 | 1.2 | | −1.0 | 1.1 | 1.8 | 1.4 | −2.1 | 0.4 |
| CD247 | J04132.1 | 1.1 | 1.0 | −1.4 | 1.3 | −1.1 | | 1.3 | 1.4 | 1.2 | −1.4 | −0.1 | 2.6 |
| CD28 | NM_006139.1 | 2.4 | 2.1 | 1.6 | −1.0 | 1.1 | | 1.6 | 1.1 | −1.9 | 1.1 | 0.4 | −3.1 |
| CD3E | NM_000733.2 | 1.1 | −1.2 | −1.1 | −1.0 | 1.4 | | 1.1 | 1.2 | −1.0 | −1.1 | 0.0 | 0.1 |
| CD3G | NM_000073.1 | −1.5 | 1.4 | 1.0 | 1.3 | −1.2 | | −1.1 | −1.1 | 1.3 | −1.1 | 0.0 | 2.4 |
| CD40 | NM_152854.1 | | | | | | | −1.7 | 1.0 | 1.2 | 1.2 | −2.7 | 0.0 |
| CD40LG | NM_000074.1 | | | | | | | −1.9 | −1.7 | −1.4 | 1.7 | −0.2 | −3.1 |
| CD69 | NM_001781.1 | 1.0 | 1.0 | 1.2 | −1.3 | 1.2 | | −3.4 | −2.5 | −1.5 | −1.4 | −0.8 | −0.1 |
| CD80 | NM_005191.1 | −1.0 | 1.0 | 1.1 | 1.1 | −1.0 | | 2.4 | 1.7 | 1.7 | 1.8 | 0.7 | −0.1 |
| CD86 | NM_006889.1 | −1.0 | −1.1 | 1.3 | −1.7 | 1.1 | | 1.1 | 1.1 | 1.2 | 1.3 | 0.0 | −0.1 |
| CDKN1A | U03106.1 | 1.2 | −1.3 | 1.1 | 1.2 | −1.7 | | 1.7 | 1.0 | 1.1 | 1.2 | 0.7 | −0.1 |
| CDKN1B | NM_004064.2 | −1.1 | 1.3 | 1.1 | 1.1 | −1.1 | | −1.4 | −1.3 | −1.8 | −1.7 | 0.0 | −0.2 |
| CDKN2B | U17075.1 | −1.2 | 1.3 | −1.1 | 1.2 | −1.0 | | 1.2 | 1.2 | 1.1 | 1.1 | 0.0 | 0.1 |
| CEBPB | NM_005194.2 | −1.2 | −1.0 | −1.0 | 1.0 | −1.0 | | 1.3 | 1.2 | 1.3 | 1.3 | 0.1 | 0.0 |
| CFB | NM_001710.3 | −1.1 | −1.3 | −1.1 | 1.0 | −1.0 | | −3.1 | −3.2 | −1.2 | 1.2 | 0.1 | −2.4 |
| CNTF | NM_000614.2 | * | * | * | * | * | | 2.8 | 2.5 | −1.1 | 1.6 | 0.3 | −2.7 |
| CNTFR | M73238.1 | −1.4 | 1.0 | 1.1 | −1.1 | −1.1 | | 1.1 | −1.1 | 1.5 | 1.4 | 2.2 | 0.1 |
| COL1A1 | NM_000088.2 | 1.0 | −1.0 | 1.4 | 1.0 | −1.8 | | −1.1 | −1.1 | 1.0 | 1.0 | 0.0 | 0.0 |
| COL1A2 | NM_000089.2 | 1.0 | −1.0 | 1.0 | 1.0 | 1.3 | | −2.6 | −1.6 | −1.6 | −1.6 | −0.9 | 0.0 |
| COL3A1 | NM_000090.2 | 1.0 | −1.1 | −1.8 | 1.1 | −1.0 | | −4.4 | −5.3 | −4.8 | −5.7 | 0.9 | 1.0 |
| CRHR1 | NM_004382.2 | 1.0 | −1.2 | 1.1 | −1.7 | 1.3 | | −1.5 | −1.8 | −1.7 | −3.7 | 0.3 | 2.0 |
| CRP | NM_000567.2 | −1.0 | 1.0 | 1.5 | 1.0 | 1.1 | | −1.1 | −1.9 | −1.6 | −1.9 | 0.9 | 0.3 |
| CSF1 | M37435.1 | −1.1 | −1.1 | −1.3 | 1.2 | −1.1 | | 1.2 | 1.2 | 1.1 | 1.1 | 0.0 | 0.1 |
| CSF1R | NM_005211.1 | * | * | * | * | * | | 1.1 | 1.5 | 1.3 | 1.3 | −0.4 | 0.0 |
| CSF2 | M11220.1 | 2.4 | 1.0 | −1.1 | −1.0 | 1.2 | | 1.1 | 1.1 | 1.1 | 1.2 | 0.0 | −0.1 |
| CSF3 | X03438.1 | 1.7 | 1.0 | 1.2 | 1.0 | −1.0 | | 1.9 | 1.8 | −1.9 | 1.1 | 0.1 | −3.0 |
| CSF3R | M59820.1 | 1.0 | −1.1 | −1.3 | 1.1 | 1.2 | | 1.2 | −1.1 | 1.2 | 1.7 | 2.2 | −0.4 |
| CTGF | U14750.1 | 1.1 | 1.4 | 1.4 | 1.1 | −1.2 | | 1.4 | 1.3 | 2.7 | 2.3 | 0.0 | 0.3 |
| CTLA4 | NM_005214.2 | 1.0 | 1.2 | 1.0 | 1.1 | 1.0 | | −1.4 | 1.2 | 1.7 | 1.9 | −2.6 | −0.2 |
| CX3CR1 | NM_001337.2 | −1.0 | 1.9 | 2.1 | 1.0 | 1.1 | | −1.1 | −1.4 | 1.9 | −1.4 | 0.4 | 3.3 |
| CXCL12 | NM_000609.3 | 1.0 | 1.1 | 1.0 | 1.0 | 1.3 | | −1.4 | −1.5 | 1.2 | 1.2 | 0.1 | 0.0 |
| CXCL2 | NM_002089.1 | 1.6 | 1.3 | −1.4 | 1.1 | 1.3 | | 1.3 | −1.1 | 1.1 | 1.1 | 2.4 | 0.0 |
| CXCR4 | NM_003467.2 | 1.2 | 1.0 | −1.4 | 1.0 | 1.0 | | −1.3 | 1.4 | −1.1 | −1.1 | −2.7 | 0.0 |
| DDR1 | L11315.1 | −1.2 | 1.2 | 1.0 | 1.0 | 1.4 | | 2.1 | 2.0 | 1.3 | 1.3 | 0.0 | 0.0 |
| EDN2 | M65199.1 | 1.2 | 1.0 | −1.2 | 1.2 | −1.1 | | 1.1 | 1.0 | −1.1 | 1.1 | 0.0 | −2.2 |
| EDN3 | J05081.1 | 1.2 | 1.0 | 1.2 | 1.0 | 1.0 | | 1.2 | 1.1 | −1.2 | 1.1 | 0.1 | −2.3 |
| EDNRB | S57283.1 | −1.0 | 1.0 | −1.2 | −1.8 | −1.1 | | −1.1 | −1.2 | 1.2 | 1.1 | 0.1 | 0.1 |
| EGF | NM_001963.2 | 1.0 | 1.0 | −1.1 | 1.3 | 1.1 | | −1.2 | 1.2 | 1.7 | −1.2 | −2.4 | 3.0 |
| EGFR | NM_005228.1 | 1.8 | −1.0 | 1.0 | −1.0 | −1.2 | | −1.5 | −1.6 | 1.1 | 1.1 | 0.1 | 0.0 |
| EGR1 | NM_001964.1 | * | * | * | * | * | | 1.3 | 1.2 | −1.3 | −1.6 | 0.1 | 0.2 |
| EGR2 | NM_000399.2 | 1.0 | 1.7 | −1.3 | −1.9 | −1.6 | | −6.6 | −2.5 | −1.8 | 1.0 | −4.1 | −2.9 |
| EGR3 | NM_004430.1 | −1.0 | 1.4 | 1.1 | 1.2 | 1.1 | | −1.6 | −1.4 | 1.9 | 1.5 | −0.2 | 0.3 |
| ENG | NM_000118.1 | 1.0 | −1.2 | −1.2 | 1.1 | 1.9 | | −2.2 | −2.6 | 1.6 | −1.5 | 0.4 | 3.1 |
| EP300 | NM_001429.2 | 1.1 | −2.4 | −1.2 | 1.0 | −1.1 | | 1.7 | 1.7 | −1.3 | −1.0 | −0.1 | −0.3 |
| EPHA3 | AF213459.1 | 1.8 | 1.7 | 1.1 | −1.3 | 1.1 | | −1.1 | −1.3 | 1.2 | 1.1 | 0.3 | 0.1 |
| EPHB1 | NM_004441.3 | 1.1 | −1.0 | 1.2 | 1.0 | 1.0 | | 4.6 | 4.0 | 5.2 | 5.0 | 0.6 | 0.2 |
| EPHB2 | AF025304.1 | −1.0 | −1.0 | 1.2 | 1.7 | 1.0 | | 1.1 | −1.4 | −1.1 | 1.0 | 2.5 | −2.1 |
| EPO | NM_000799.1 | −1.0 | −1.9 | −1.1 | 1.4 | 1.2 | | −1.8 | −2.4 | −1.8 | −1.9 | 0.6 | 0.2 |
| EPOR | NM_000121.2 | −1.0 | 1.0 | 1.0 | −1.3 | 1.0 | | 1.6 | 1.5 | −1.3 | −1.4 | 0.1 | 0.1 |
| ERBB2 | NM_004448.1 | −1.0 | −1.2 | 1.3 | −1.2 | 1.1 | | −1.2 | −1.3 | 1.4 | 1.4 | 0.1 | 0.1 |
| ERBB3 | M29366.1 | 2.2 | 1.0 | 1.0 | 1.1 | 1.1 | | 1.0 | −1.0 | −2.3 | −1.8 | 2.0 | −0.5 |
| ERBB4 | NM_005235.1 | −1.1 | 1.0 | 1.0 | 1.0 | 1.1 | | 1.2 | 1.0 | −1.0 | −1.5 | 0.2 | 0.4 |
| F2R | NM_001992.2 | −1.1 | −1.1 | 1.3 | 1.0 | −1.0 | | 1.4 | 1.4 | 1.6 | 1.4 | −0.1 | 0.2 |
| FADD | NM_003824.2 | 1.0 | 1.0 | 1.3 | 1.4 | 2.5 | | 1.1 | −1.0 | 1.0 | −1.0 | 2.1 | 2.1 |
| FAS | NM_000043.3 | | | | | | | −1.0 | −1.0 | −1.2 | 1.1 | 0.0 | −2.3 |
| FASLG | NM_000639.1 | | | | | | | 1.2 | 1.3 | 1.1 | 1.4 | −0.1 | −0.3 |
| FCER2 | M14766.1 | 1.1 | 1.0 | 1.0 | 1.3 | 1.0 | | −1.4 | −1.7 | 1.5 | 1.4 | 0.3 | 0.1 |
| FCGR3A | NM_000569.6 | −1.1 | 1.0 | −1.7 | 1.0 | −1.4 | | 2.1 | 2.5 | 1.3 | 1.3 | −0.4 | 0.0 |
| FGF1 | X51943.1 | −1.0 | 1.3 | 1.2 | 1.2 | 1.0 | | −1.0 | −1.4 | 2.4 | 2.2 | 0.4 | 0.2 |
| FGF2 | NM_002006.3 | 1.0 | 1.2 | −1.2 | 1.1 | 1.1 | | −1.1 | 1.1 | 1.4 | 1.2 | −2.2 | 0.3 |
| FGF3 | NM_005247.2 | 1.0 | 1.2 | 1.1 | 1.4 | −1.1 | | −3.6 | −1.1 | 2.1 | 1.1 | −2.5 | 1.1 |
| FGF5 | AF535149.1 | 1.0 | 1.0 | −1.3 | −1.3 | −1.3 | | −1.0 | 1.1 | 2.5 | 2.7 | −2.1 | −0.2 |
| FGF6 | NM_020996.1 | −1.3 | 1.0 | −1.2 | 1.2 | 1.2 | | 1.5 | 1.3 | −1.0 | 1.1 | 0.2 | −2.2 |
| FGF7 | NM_002009.2 | 1.0 | 1.1 | −1.1 | −1.1 | 1.1 | | 1.2 | −1.4 | 1.0 | −1.3 | 2.6 | 2.3 |
| FGF8 | U36223.1 | −1.1 | −1.2 | 1.1 | 1.3 | −1.0 | | −1.0 | 1.3 | −1.5 | −1.5 | −2.3 | 0.0 |
| FGF9 | NM_002010.1 | 1.4 | 1.2 | 1.6 | 1.2 | −1.4 | | 1.0 | −1.1 | −1.2 | −1.2 | 2.1 | 0.0 |
| FGFR1 | NM_000604.2 | 1.0 | 1.1 | 1.2 | 1.6 | −1.0 | | −1.1 | −1.0 | 1.4 | 1.5 | 0.0 | −0.1 |
| FGFR2 | M87770.1 | 1.5 | 1.0 | 1.3 | −1.1 | −1.2 | | 1.3 | 1.2 | 1.2 | 1.0 | 0.1 | 0.2 |
| FGFR3 | M58051.1 | 1.0 | −1.5 | 1.0 | −1.7 | 1.0 | | 1.0 | 1.1 | −1.3 | 1.0 | 0.0 | −2.3 |
| FGFR4 | L03840.1 | 1.0 | 1.4 | 1.2 | 1.0 | 1.3 | | 1.2 | 1.5 | 1.0 | 1.1 | −0.3 | −0.1 |
| FLT1 | NM_002019.2 | * | * | * | * | * | | 2.5 | 1.0 | −1.6 | −2.0 | 1.5 | 0.4 |
| FLT3 | Z26652.1 | −1.0 | 1.0 | 1.4 | 1.1 | 1.3 | | 1.3 | −1.2 | 2.0 | 1.5 | 2.4 | 0.5 |
| FLT4 | NM_002020.1 | 1.1 | −1.5 | −1.1 | −1.0 | −1.4 | | −1.1 | 1.1 | −1.1 | 1.3 | −2.3 | −2.3 |
| FOS | NM_005252.2 | 1.0 | −1.4 | 1.4 | 1.2 | −1.1 | | 1.4 | 1.3 | 1.1 | −1.0 | 0.1 | 2.1 |
| FOSL1 | NM_005438.2 | 1.0 | −1.2 | −1.3 | 1.1 | 1.6 | | 2.2 | 2.2 | 2.7 | 2.5 | 0.0 | 0.3 |
| FSHR | NM_000145.2 | −1.1 | 2.2 | −1.4 | 1.0 | 1.0 | | −1.8 | −2.1 | 1.8 | −1.2 | 0.3 | 3.0 |
| FST | NM_006350.2 | −1.2 | −1.2 | −1.2 | −1.5 | −1.0 | | −2.9 | −2.4 | 1.8 | 2.0 | −0.6 | −0.2 |
| GAPDH | NM_002046.2 | −1.1 | −1.4 | −1.5 | 1.1 | 1.1 | 1.1 | 1.0 | 1.0 | −1.0 | −1.0 | 0.0 | 0.0 |
| GATA3 | NM_002051.1 | 1.0 | −1.2 | −1.1 | −1.3 | 1.2 | | 1.0 | 1.0 | 1.1 | −1.1 | 0.0 | 2.2 |
| GATA4 | NM_002052.2 | 1.1 | 1.0 | 1.1 | −1.9 | −1.0 | | −1.3 | −1.3 | −1.3 | −1.2 | 0.0 | −0.2 |
| GDNF | NM_199231.1 | 1.4 | −1.3 | −1.3 | −1.2 | 1.0 | | 2.4 | 1.6 | 1.8 | 1.9 | 0.8 | −0.1 |
| HBEGF | M60278.1 | −1.4 | −1.9 | 1.3 | −1.8 | −1.0 | | −1.1 | −1.1 | 1.1 | −1.1 | 0.0 | 2.1 |
| HGF | X16323.1 | 1.1 | 1.3 | −1.1 | 1.2 | 1.0 | | 1.1 | 1.2 | 1.4 | 1.2 | −0.2 | 0.2 |
| HIF1A | U29165.1 | 1.0 | 1.4 | −1.5 | 1.0 | 1.1 | | 1.0 | 1.1 | 1.5 | 1.8 | −0.1 | −0.3 |
| HK1 | M75126.1 | −1.1 | −1.1 | 1.0 | 1.4 | −1.1 | | 1.6 | 1.3 | 1.7 | 1.5 | 0.3 | 0.2 |
| HPRT1 | NM_000194.1 | 1.2 | 1.3 | 1.0 | 1.2 | −1.7 | 1.4 | −2.1 | −2.0 | −1.4 | −1.3 | −0.1 | −0.2 |
| HRAS | NM_005343.2 | −1.1 | −1.1 | −1.1 | 1.0 | 1.1 | | 1.9 | 1.6 | 1.1 | 1.2 | 0.3 | −0.1 |
| ICAM1 | J03132.1 | −1.0 | 1.1 | 1.1 | −1.2 | −1.0 | | 1.6 | 1.6 | 2.3 | 2.3 | 0.0 | 0.0 |
| ICAM2 | NM_000873.2 | 1.0 | 1.3 | −1.1 | −1.2 | 1.8 | | 1.5 | 1.3 | 1.4 | −1.1 | 0.2 | 2.5 |
| ICAM3 | NM_002162.2 | −1.0 | −1.1 | −1.7 | 1.0 | 1.5 | | 1.5 | −1.5 | 2.4 | 1.8 | 3.0 | 0.6 |
| IFNAR1 | NM_000629.1 | 1.0 | −1.2 | 1.2 | −1.1 | 1.4 | | −1.8 | −1.7 | −1.5 | −1.7 | −0.1 | 0.1 |
| IGF1 | X57025.1 | 1.0 | 1.0 | 1.2 | 1.4 | 1.2 | −1.2 | −1.0 | −1.0 | −1.4 | −1.3 | 0.0 | 0.0 |
| IGF1R | NM_000875.2 | −1.1 | −1.2 | −1.5 | 1.2 | 1.0 | | −2.7 | −2.6 | −1.5 | −1.5 | −0.2 | 0.0 |
| IGF2 | S77035.1 | 1.0 | −1.7 | 1.4 | −1.3 | 1.3 | 1.1 | 1.2 | 1.9 | −2.0 | −1.3 | −0.6 | −0.7 |
| IGFBP1 | NM_000596.1 | 1.1 | 2.1 | −1.2 | 1.1 | 1.1 | | 2.2 | −1.9 | −1.3 | −1.4 | 4.1 | 0.0 |
| IGFBP2 | M35410.1 | 1.3 | 1.2 | 1.0 | 1.4 | 1.1 | | −2.1 | 1.0 | 1.2 | 1.2 | −3.1 | −0.1 |
| IGFBP3 | X64875.1 | −1.3 | −1.1 | 1.2 | −1.6 | −1.3 | | −1.0 | −1.1 | 1.1 | 1.1 | 0.0 | 0.0 |
| IGFBP4 | NM_001552.1 | 1.0 | −1.6 | 1.3 | 1.5 | 2.8 | | −1.1 | 1.1 | −1.1 | −1.3 | −2.2 | 0.2 |
| IGFBP5 | M65062.1 | 1.0 | 1.2 | 1.1 | 1.4 | −1.1 | | −3.9 | −3.3 | 1.1 | 1.3 | −0.6 | −0.2 |
| IGFBP6 | M69054.1 | 1.0 | −1.4 | −1.0 | 1.0 | 1.3 | | −1.3 | −1.3 | −1.2 | −1.1 | 0.0 | 0.0 |
| IL10 | NM_000572.1 | 1.1 | 1.1 | 1.2 | 1.0 | −1.5 | −2.8 | 3.0 | 2.2 | 1.1 | 1.2 | 0.8 | 0.0 |
| IL10RA | NM_001558.2 | 1.0 | −1.1 | −1.4 | −4.7 | 1.0 | | 1.3 | 1.4 | 1.3 | 1.3 | −0.1 | 0.0 |
| IL11 | NM_000641.1 | −1.1 | 1.1 | 1.2 | 1.4 | −1.2 | | 3.3 | 3.1 | 4.2 | 5.2 | 0.2 | −1.0 |
| IL13 | NM_002188.2 | −1.1 | 1.0 | 1.1 | −1.0 | −1.2 | | −1.1 | −1.0 | −1.0 | −1.3 | −0.1 | 0.3 |
| IL13RA2 | NM_000640.2 | 1.4 | 1.0 | 1.0 | 1.2 | 1.1 | | 1.3 | 1.4 | −1.0 | 1.1 | −0.2 | −2.1 |
| IL15 | NM_000585.1 | 1.2 | 1.2 | −1.0 | 2.3 | 1.1 | | −1.4 | −1.3 | −1.6 | −1.4 | −0.1 | −0.2 |
| IL16 | U82972.1 | −1.4 | 1.0 | 1.0 | 1.1 | 1.3 | | −1.6 | −1.3 | −1.2 | 2.0 | −0.3 | −3.1 |
| IL17 | U32659.1 | * | * | * | * | * | | −1.6 | −1.3 | −1.2 | 2.0 | −0.3 | −3.1 |
| IL18 | NM_001562.2 | 1.0 | 1.2 | 1.0 | 1.1 | −1.2 | | −1.2 | −2.3 | −1.6 | −1.1 | 1.2 | −0.6 |
| IL1A | NM_000575.1 | −1.0 | 1.0 | 1.1 | 1.0 | −1.0 | −5.3 | 1.1 | −1.1 | 1.1 | 1.1 | 2.2 | −0.1 |
| IL1B | M15330.1 | * | * | * | * | * | | 1.5 | 1.6 | −1.6 | −1.1 | −0.1 | −0.5 |
| IL1R1 | NM_000877.2 | 1.1 | −1.2 | −1.3 | 1.0 | −1.3 | | 1.4 | 1.9 | 1.3 | 1.7 | −0.5 | −0.4 |
| IL1RL1 | AB012701.2 | 1.2 | −2.6 | 1.0 | −1.2 | 1.1 | | −1.2 | −1.0 | 1.1 | 1.1 | −0.2 | 0.0 |
| IL1RN | X52015.1 | 1.1 | 1.0 | 1.2 | 1.2 | −1.1 | | 1.3 | 1.5 | 1.2 | 1.3 | −0.1 | −0.1 |
| IL2 | U25676.1 | −1.1 | 2.2 | 1.6 | 1.1 | 1.0 | | −1.3 | −1.4 | 1.1 | −1.3 | 0.2 | 2.4 |
| IL23A | NM_016584.2 | −2.1 | 1.0 | 1.0 | 1.1 | 1.2 | | 1.5 | −1.2 | −1.2 | −1.4 | 2.7 | 0.2 |
| IL2RA | NM_000417.1 | 1.2 | 1.0 | −1.3 | 1.0 | −1.1 | | −1.9 | 1.1 | 1.3 | 1.3 | −3.0 | 0.0 |
| IL2RB | NM_000878.2 | 1.5 | 1.0 | 1.1 | −1.3 | −1.1 | | −1.9 | −1.5 | 1.1 | 1.1 | −0.3 | −0.1 |
| IL2RG | NM_000206.1 | −1.0 | 1.6 | 1.2 | 1.0 | −1.0 | | 1.1 | 1.1 | −1.0 | −1.1 | 0.0 | 0.1 |
| IL3 | M20137.1 | −1.1 | 1.0 | −1.2 | 1.1 | −1.1 | | 1.5 | 1.1 | 1.4 | −1.3 | 0.3 | 2.7 |
| IL4 | NM_000589.1 | 1.0 | 1.4 | 1.3 | 1.0 | −1.1 | | −2.8 | −1.2 | 1.3 | 1.1 | −1.6 | 0.2 |
| IL4R | NM_000418.1 | −1.1 | 1.2 | 1.0 | 1.0 | 1.1 | | −1.2 | 1.0 | −1.2 | −1.1 | −2.2 | 0.0 |
| IL5 | NM_000879.2 | −1.6 | 1.3 | 1.2 | 1.4 | 3.1 | | 2.4 | 3.8 | 1.2 | 2.2 | −1.5 | −1.0 |
| IL5RA | NM_175726.1 | 1.0 | 1.0 | −1.4 | −1.5 | −1.2 | | −1.0 | 1.2 | 1.2 | 1.1 | −2.3 | 0.1 |
| IL6 | NM_000600.1 | −1.0 | 1.5 | −1.3 | 1.0 | 1.0 | −1.5 | −1.2 | −1.0 | 2.6 | 2.8 | −0.2 | −0.1 |
| IL6R | NM_000565.1 | 1.1 | −1.0 | 1.6 | 1.0 | 1.1 | | 1.9 | 1.5 | 1.0 | 1.2 | 0.4 | −0.2 |
| IL7 | NM_000880.2 | −1.1 | −1.0 | 1.2 | 1.2 | 1.0 | | −2.3 | −1.3 | −1.5 | −1.3 | −1.0 | −0.2 |
| IL7R | NM_002185.2 | −1.0 | 1.3 | 1.2 | 1.1 | −1.2 | | −2.0 | −1.5 | 1.6 | 1.6 | −0.4 | 0.0 |
| IL8 | NM_000584.1 | −1.2 | 1.0 | 1.0 | 1.0 | 1.1 | | −2.0 | 21.7 | 14.8 | 15.9 | −23.7 | −1.1 |
| IL8RA | NM_000634.2 | 1.1 | −1.7 | −1.0 | 1.0 | −1.0 | | 1.2 | 1.2 | 1.0 | −1.1 | 0.0 | 2.1 |
| IL8RB | NM_001557.2 | 1.4 | 1.0 | 1.3 | 1.7 | 1.3 | | 1.0 | 1.5 | −1.0 | 1.1 | −0.5 | −2.1 |
| IL9 | NM_000590.1 | −1.0 | 1.0 | 1.0 | 1.2 | −1.0 | | 1.2 | 1.1 | −1.8 | −2.6 | 0.2 | 0.8 |
| IRAK1 | NM_001569.2 | 1.0 | −1.4 | −1.1 | 1.0 | −1.1 | | 1.2 | 1.2 | 1.4 | 1.2 | 0.0 | 0.2 |
| IRF1 | NM_002198.1 | 1.0 | −1.2 | −1.6 | 1.0 | 1.2 | | 1.1 | 1.1 | 1.2 | 1.1 | 0.0 | 0.1 |
| IRF2 | NM_002199.2 | 1.2 | 1.3 | 1.0 | −1.1 | −1.1 | | −2.1 | −1.4 | −2.5 | −1.0 | −0.8 | −1.4 |
| IRF3 | NM_001571.2 | 1.0 | −1.4 | −1.0 | 1.0 | 1.3 | | 1.1 | 1.1 | 1.1 | 1.1 | 0.0 | 0.0 |
| IRF7 | NM_001572.2 | −1.1 | 1.1 | 1.4 | 1.3 | −1.0 | | 1.3 | 1.1 | −1.4 | −1.2 | 0.2 | −0.1 |
| ISGF3G | NM_006084.3 | | | | | | | −1.1 | 1.1 | 3.2 | 2.9 | −2.2 | 0.4 |
| ITGB2 | NM_000211.1 | −1.0 | −1.3 | 1.0 | 1.0 | 1.1 | | −1.1 | 1.7 | −1.1 | −1.2 | −2.8 | 0.1 |
| ITK | NM_005546.3 | 1.0 | 1.0 | −1.0 | −1.1 | 1.1 | | −1.0 | 1.0 | 1.1 | 1.0 | −2.0 | 0.1 |
| JAK1 | NM_002227.1 | 1.1 | 1.0 | 1.1 | −1.1 | 1.0 | | −1.3 | 1.1 | −1.8 | −3.5 | −2.4 | 1.7 |
| JAK2 | NM_004972.2 | −1.1 | 1.1 | −1.3 | 1.3 | 1.1 | | −1.5 | −1.0 | 1.2 | 1.0 | −0.5 | 0.2 |
| JAK3 | NM_000215.2 | 1.0 | 1.0 | −1.1 | −1.1 | −1.0 | | −1.1 | −1.1 | 1.2 | 1.0 | 0.0 | 0.1 |
| JUN | NM_002228.3 | 1.0 | −1.1 | −1.1 | −1.6 | 1.2 | | 1.4 | 2.2 | 1.8 | 2.0 | −0.8 | −0.2 |
| K-ALPHA-1 | NM_006082.1 | * | * | * | * | * | | 1.8 | 1.6 | 1.3 | 1.2 | 0.2 | 0.1 |
| KDR | NM_002253.1 | −1.2 | 1.0 | 1.0 | −1.1 | 1.0 | | 1.6 | 1.9 | −1.1 | 1.0 | −0.3 | −2.1 |
| KIT | NM_000222.1 | −1.3 | 1.4 | −1.2 | −1.1 | −1.0 | | −87.7 | −26.7 | −1.4 | −1.6 | −60.9 | 0.2 |
| KITLG | NM_000899.1 | −1.4 | −1.4 | 1.1 | 1.0 | 1.1 | | −2.5 | −2.9 | −1.6 | −1.4 | 0.4 | −0.2 |
| KLK3 | NM_001648.2 | 1.4 | −1.0 | 1.2 | 1.0 | 1.3 | | −2.2 | 1.1 | 1.2 | 1.2 | −3.3 | 0.0 |
| LAMP1 | NM_005561.2 | 1.1 | 1.1 | 1.0 | 1.3 | −1.2 | | −1.3 | −1.1 | 1.0 | 1.0 | −0.1 | 0.0 |
| LAT | NM_014387.2 | 1.1 | −1.1 | 1.0 | −1.1 | −1.1 | | −1.5 | −1.1 | 1.0 | 1.4 | −0.4 | −0.4 |
| LIF | NM_002309.2 | 1.1 | −2.0 | −1.1 | 1.1 | −1.0 | | 1.9 | 1.8 | 2.3 | 2.0 | 0.1 | 0.3 |
| LILRB4 | NM_006847.2 | 1.0 | −1.0 | 1.1 | −1.4 | −1.2 | | −1.1 | 1.5 | −1.4 | 1.2 | −2.6 | −2.6 |
| LTA | NM_000595.2 | 1.9 | 2.0 | −1.1 | −1.2 | 1.4 | | 1.4 | 1.8 | −2.2 | 1.0 | −0.4 | −3.2 |
| LTBR | NM_002342.1 | 1.4 | −1.4 | 1.1 | −1.1 | 1.0 | | 1.1 | 1.7 | 1.7 | 1.7 | −0.6 | 0.0 |
| MAP2K1 | NM_002755.2 | −1.1 | −1.3 | 1.2 | 1.5 | −1.1 | | 1.5 | 1.4 | 1.4 | 1.3 | 0.1 | 0.0 |
| MAP2K4 | NM_003010.1 | −1.0 | 1.3 | 1.0 | 1.6 | −1.0 | | 1.1 | 1.1 | −1.1 | −1.1 | 0.1 | 0.0 |
| MAP2K7 | AF013589.1 | 1.0 | 1.8 | 1.0 | −1.1 | 1.0 | | −1.5 | −1.0 | −1.3 | −1.1 | −0.5 | −0.1 |
| MAP3K1 | XM_042066.9 | −1.1 | 1.6 | −1.5 | −1.3 | −1.3 | | −2.0 | −2.1 | −1.4 | −1.4 | 0.1 | 0.0 |
| MAP3K14 | NM_003954.1 | −1.0 | −1.1 | 1.1 | −1.1 | −1.1 | | −1.4 | −1.1 | −1.2 | 1.2 | −0.3 | −2.5 |
| MAP3K7 | AB009357.1 | 1.1 | 1.0 | −1.3 | 1.0 | −1.2 | | −2.5 | −1.2 | 1.1 | 1.1 | −1.3 | 0.0 |
| MAP3K8 | NM_005204.2 | 1.0 | −1.0 | −1.0 | 1.0 | 1.1 | | 1.5 | 2.1 | 1.3 | 1.2 | −0.7 | 0.1 |
| MAPK14 | AF100544.1 | 1.0 | −1.4 | −1.1 | 1.0 | −1.2 | 2.3 | −1.3 | −1.3 | 1.1 | 1.1 | 0.0 | −0.1 |
| MAPK3 | X60188.1 | 1.5 | −1.4 | −1.5 | 1.0 | 1.6 | | −1.2 | −1.4 | −1.0 | −1.1 | 0.2 | 0.1 |
| MAPK8 | L26318.1 | −1.2 | 1.4 | 1.4 | 1.3 | 1.1 | | −1.5 | 1.7 | 1.3 | 1.5 | −3.2 | −0.2 |
| MAPK9 | U09759.1 | −1.0 | −1.4 | 1.0 | 1.2 | 1.3 | | −1.2 | −1.1 | −1.0 | 1.0 | 0.0 | −2.0 |
| MDH1 | NM_005917.1 | −1.1 | 1.2 | 1.8 | 1.2 | 1.2 | | −1.4 | −1.2 | −1.4 | −1.5 | −0.1 | 0.1 |
| MET | NM_000245.1 | −1.2 | 1.3 | 1.0 | 1.4 | 1.0 | | 1.3 | 1.9 | 1.6 | 1.7 | −0.6 | −0.1 |
| MIF | NM_002415.1 | 1.0 | −1.1 | −1.2 | 1.0 | −1.7 | | 1.3 | 1.2 | 1.0 | 1.0 | 0.1 | 0.0 |
| MMP2 | NM_004530.1 | −1.1 | −1.1 | 1.1 | −1.2 | 1.1 | | −1.4 | 1.2 | 1.2 | 1.0 | −2.6 | 0.1 |
| MMP25 | AJ272137.1 | 1.0 | −1.1 | 1.2 | −1.0 | 1.5 | | 1.0 | −1.0 | 1.1 | −1.3 | 2.1 | 2.4 |
| MMP7 | NM_002423.2 | −1.1 | 1.0 | −1.2 | 1.2 | 1.1 | | −1.2 | −1.2 | −1.3 | −1.4 | 0.1 | 0.1 |
| MST1 | NM_020998.2 | −2.1 | −1.1 | 1.2 | 1.1 | 1.2 | | −3.8 | −1.9 | −1.7 | 1.0 | −2.0 | −2.7 |
| MYC | NM_002467.2 | 1.0 | 1.2 | −1.2 | −1.3 | −1.1 | | 1.2 | 1.5 | 1.5 | 1.4 | −0.2 | 0.0 |
| NBL1 | NM_005380.3 | 1.0 | −1.7 | 1.2 | −1.0 | 7.1 | | −1.6 | 1.0 | 1.8 | 1.5 | −2.7 | 0.3 |
| NCAM1 | NM_181351.1 | −1.0 | −1.3 | 1.1 | 1.1 | 1.2 | | −2.0 | −1.5 | −1.3 | −1.2 | −0.5 | −0.1 |
| NFATC1 | U80917.1 | 1.0 | 1.1 | −1.6 | 1.0 | −1.2 | | −3.5 | −1.1 | 1.4 | 1.1 | −2.4 | 0.3 |
| NFATC2 | NM_173091.1 | 1.0 | 1.0 | 1.5 | −1.1 | −1.8 | | −1.6 | 1.3 | 1.2 | 1.4 | −2.9 | −0.2 |
| NFKB1 | NM_003998.1 | −1.5 | −1.1 | 1.0 | −1.2 | 1.1 | 2.6 | −1.3 | −1.4 | 1.1 | −1.0 | 0.1 | 2.2 |
| NGFB | NM_002506.1 | −1.1 | −1.1 | −1.1 | 1.0 | −1.0 | | 1.1 | 1.2 | 1.3 | 1.5 | −0.1 | −0.2 |
| NGFR | M14764.1 | −1.1 | 1.0 | −2.0 | −1.2 | −1.0 | | 3.4 | 2.7 | 1.1 | 2.0 | 0.7 | −0.9 |
| NRG1 | M94165.1 | 1.4 | 1.1 | −1.1 | 1.0 | −1.6 | | 1.2 | 3.9 | 1.2 | −1.0 | −2.8 | 2.2 |
| NTF3 | NM_002527.2 | 1.9 | 1.1 | −1.4 | 1.1 | 1.1 | | −1.3 | −1.2 | −1.1 | −1.1 | −0.1 | −0.1 |
| NTF5 | NM_006179.3 | 1.1 | 1.1 | 1.6 | −1.1 | 1.3 | | 1.6 | −3.5 | −2.9 | −3.8 | 5.1 | 0.9 |
| NTRK1 | NM_002529.2 | 1.4 | 1.0 | −1.3 | −1.1 | 1.9 | | −1.7 | −1.9 | 1.1 | 2.0 | 0.1 | −0.9 |
| ORM1 | NM_000607.1 | −1.1 | −1.8 | −1.0 | −1.1 | −1.4 | | −1.1 | 1.3 | 1.6 | 1.6 | −2.4 | 0.0 |
| OSM | NM_020530.3 | 1.0 | −1.6 | −1.1 | 1.0 | 1.2 | | 1.4 | 1.3 | −1.9 | −1.9 | 0.1 | 0.0 |
| P2RX7 | NM_002562.4 | −1.2 | 1.0 | 1.0 | −1.2 | 1.3 | | −1.5 | −1.6 | −1.9 | −1.7 | 0.0 | −0.2 |
| PDGFA | X06374.1 | −1.2 | 1.4 | −1.3 | 1.0 | 1.0 | | −2.1 | 1.3 | 1.5 | 2.1 | −3.5 | −0.6 |
| PDGFB | NM_002608.1 | 1.5 | −2.9 | −1.0 | −1.9 | 1.1 | | −1.1 | 1.8 | −1.0 | −2.2 | −3.0 | 1.2 |
| PDGFRA | NM_006206.2 | 1.1 | −1.1 | 1.3 | 1.1 | −1.1 | | −3.3 | −1.1 | 4.5 | 2.5 | −2.3 | 1.9 |
| PDGFRB | NM_002609.2 | 1.1 | 1.1 | −1.0 | −1.3 | 1.0 | | −2.0 | −1.7 | −1.1 | −1.2 | −0.2 | 0.0 |
| PF4 | NM_002619.1 | −1.2 | 2.5 | 1.4 | 1.4 | 1.3 | | 4.1 | 3.7 | 1.6 | 1.7 | 0.4 | −0.2 |
| PGF | NM_002632.3 | −1.3 | −1.3 | −1.3 | −1.1 | 1.9 | | 1.4 | 3.4 | 4.3 | 4.1 | −2.0 | 0.3 |
| PLA2G2D | NM_012400.2 | −1.1 | −1.1 | 1.2 | 1.0 | −1.1 | | −2.5 | 2.0 | 3.4 | 3.8 | −4.5 | −0.3 |
| PLA2G2E | NM_014589.1 | 1.0 | 1.6 | 1.1 | −1.6 | −1.1 | | −1.4 | −1.8 | −1.2 | −1.6 | 0.4 | 0.3 |
| PLA2G4C | NM_003706.1 | −1.3 | 1.1 | 1.0 | 1.0 | 1.3 | | −1.0 | 1.1 | 3.7 | 1.7 | −2.1 | 1.9 |
| PLAA | NM_004253.2 | 1.0 | −1.1 | 1.8 | 1.0 | 1.3 | | 1.4 | 1.3 | 1.6 | 1.7 | 0.0 | −0.1 |
| PML | BT009911.1 | 1.0 | 1.0 | −1.1 | −1.1 | 1.2 | | −3.4 | −1.1 | 1.2 | −1.0 | −2.4 | 2.2 |
| PPARA | NM_005036.2 | 1.0 | −1.0 | 1.0 | −1.2 | −1.3 | | −4.5 | −3.6 | −2.5 | −2.7 | −1.0 | 0.2 |
| PPARG | NM_015869.1 | −1.0 | −1.2 | 1.5 | −1.0 | −1.1 | −1.7 | −1.3 | −1.2 | −1.1 | −1.1 | −0.1 | 0.0 |
| PPBP | NM_002704.1 | −2.3 | 1.0 | 2.1 | −1.3 | 1.2 | | 1.1 | −1.4 | −1.5 | −1.6 | 2.5 | 0.2 |
| PPIE | AF042385.1 | 1.1 | −1.2 | −1.4 | −1.0 | −1.7 | | −2.3 | −2.1 | −1.5 | −1.0 | −0.2 | −0.5 |
| PRDX5 | NM_181652.1 | 1.1 | −1.3 | 1.0 | 1.0 | −1.1 | | −1.2 | −1.2 | −1.1 | −1.1 | 0.0 | 0.0 |
| PRL | NM_000948.2 | −1.0 | 1.0 | −1.3 | 1.2 | −1.1 | | 1.6 | 1.0 | 1.1 | −1.0 | 0.6 | 2.1 |
| PTGS2 | NM_000963.1 | −1.2 | 1.3 | 1.5 | −1.3 | 1.1 | −2.0 | 1.6 | 1.9 | 6.0 | 5.8 | −0.3 | 0.2 |
| PTN | NM_002825.3 | 1.1 | 1.1 | −1.2 | 1.2 | −1.5 | | −1.4 | −1.0 | 1.0 | 1.2 | −0.4 | −0.1 |
| PTPRC | NM_002838.2 | −1.2 | 1.0 | −1.1 | 1.0 | −1.1 | | −1.4 | −1.3 | 1.6 | 1.2 | −0.1 | 0.4 |
| RENBP | NM_002910.4 | −1.0 | 1.7 | 1.0 | −1.6 | −1.6 | | 1.1 | 1.3 | 2.2 | 1.2 | −0.2 | 1.0 |
| RET | NM_000323.2 | 1.0 | 1.0 | 1.1 | 1.1 | 1.2 | | −2.0 | 1.8 | −1.8 | 1.9 | −3.8 | −3.7 |
| RPL13A | X56932.1 | 1.0 | −1.1 | 1.3 | 1.0 | 1.0 | | −1.0 | −1.0 | −1.0 | −1.0 | 0.0 | 0.0 |
| RPS9 | NM_001013.2 | −1.2 | 1.0 | −1.1 | 1.0 | −1.0 | | −1.1 | 1.3 | 1.1 | −1.2 | −2.3 | 2.3 |
| RUNX1 | D43968.1 | 1.2 | 1.5 | 1.2 | 1.0 | 1.0 | | −2.5 | 1.5 | 3.0 | 2.9 | −4.0 | 0.1 |
| RUNX2 | NM_004348.1 | 1.0 | −1.1 | −1.2 | 1.0 | 1.0 | | −1.6 | −1.1 | −1.3 | −1.3 | −0.6 | 0.0 |
| S100A8 | NM_002964.2 | −1.0 | −1.1 | −1.5 | 1.0 | 1.2 | | 1.1 | 1.3 | 1.1 | −1.3 | −0.3 | 2.4 |
| S100A9 | NM_002965.2 | 1.3 | 1.0 | −1.1 | −1.1 | −1.2 | | 3.9 | 4.2 | 1.8 | −1.9 | −0.3 | 3.7 |
| SAA1 | NM_000331.2 | 1.0 | −3.2 | 1.1 | −1.3 | −1.0 | | 1.2 | 1.8 | 1.1 | −1.1 | −0.7 | 2.1 |
| SDS | NM_006843.1 | 1.0 | 1.2 | 1.0 | 1.1 | 1.4 | | 1.2 | 1.5 | −1.2 | 1.4 | −0.2 | −2.6 |
| SELE | M30640.1 | −1.1 | 1.4 | 1.1 | 1.0 | −1.1 | | −1.7 | −1.3 | −1.2 | −2.0 | −0.4 | 0.8 |
| SELL | NM_000655.2 | 1.5 | 1.0 | 1.2 | 1.2 | 1.1 | | 1.1 | 1.2 | 1.3 | 1.2 | −0.1 | 0.1 |
| SERPINE1 | M14083.1 | 1.0 | −1.2 | −1.1 | −1.2 | −2.2 | | 1.0 | 1.2 | 1.7 | 1.6 | −0.2 | 0.0 |
| SERPINE2 | NM_006216.2 | 1.4 | −1.3 | −1.0 | 1.4 | −1.1 | | −2.0 | 1.0 | 1.5 | 1.1 | −3.0 | 0.4 |
| SLC1A2 | NM_004171.2 | 1.0 | 1.0 | −1.1 | 1.2 | 1.3 | | −7.2 | −6.7 | −2.1 | −2.0 | −0.5 | 0.0 |
| SOCS2 | AF037989.1 | 1.1 | 1.1 | 1.1 | −1.1 | 1.2 | | −1.8 | −1.5 | 1.3 | 1.1 | −0.3 | 0.2 |
| SP1 | NM_138473.2 | 1.1 | 1.0 | −1.3 | 1.2 | −1.1 | | −1.6 | −1.8 | −1.7 | −1.7 | 0.2 | −0.1 |
| SRF | NM_003131.1 | 1.0 | 1.1 | 1.1 | −1.2 | −1.3 | | −1.2 | −1.2 | 1.1 | 1.0 | 0.0 | 0.0 |
| STAT1 | NM_007315.2 | 1.1 | −1.2 | 1.0 | 1.1 | 1.1 | | −2.3 | −1.5 | 1.2 | 1.1 | −0.8 | 0.0 |
| STAT2 | NM_005419.2 | −1.1 | −1.1 | 1.1 | 2.5 | 1.0 | | −2.7 | −2.3 | −1.4 | −1.4 | −0.4 | 0.0 |
| STAT4 | NM_003151.2 | −1.0 | 1.0 | −1.6 | 1.3 | 1.1 | | −4.1 | −3.7 | −1.7 | −1.5 | −0.4 | −0.2 |
| STAT6 | NM_003153.3 | 1.0 | 1.1 | 1.3 | 1.0 | −1.2 | | −1.8 | −1.3 | 2.0 | 1.4 | −0.5 | 0.6 |
| TAP1 | NM_000593.5 | 1.0 | −1.1 | −1.4 | −1.1 | 1.3 | | −1.8 | −1.2 | −1.2 | −1.2 | −0.6 | 0.0 |
| TFRC | NM_003234.1 | 1.2 | 1.0 | 1.2 | 1.0 | 1.1 | | −2.0 | −2.0 | 1.6 | −1.8 | 0.1 | 3.4 |
| TGFB1 | NM_000660.1 | −1.1 | −1.2 | 1.1 | 1.0 | 1.0 | | 1.0 | 1.9 | 1.3 | 1.2 | −0.8 | 0.1 |
| TGFBI | NM_000358.1 | 1.3 | 1.1 | 1.1 | 1.1 | −1.1 | | 1.0 | 1.0 | −1.0 | −1.1 | 0.0 | 0.0 |
| TGFBR1 | NM_004612.1 | −1.2 | 1.6 | 1.7 | −1.2 | 1.1 | | −2.2 | −1.5 | −1.1 | 1.1 | −0.8 | −2.1 |
| THBD | NM_000361.2 | 1.0 | −1.0 | −1.2 | −1.2 | 1.3 | | 8.1 | 7.3 | 3.1 | 2.5 | 0.7 | 0.7 |
| THPO | D32047.1 | 1.0 | 1.4 | −1.5 | 1.1 | −1.3 | | −1.5 | −1.0 | −1.5 | −2.1 | −0.4 | 0.6 |
| TIMP1 | NM_003254.1 | 1.3 | −1.3 | 1.0 | −1.2 | −1.4 | | −1.0 | −1.0 | −1.0 | −1.0 | 0.0 | 0.0 |
| TLR2 | NM_003264.2 | −1.5 | 1.0 | −1.6 | −1.0 | 1.0 | | −1.2 | −1.2 | −1.0 | −1.1 | 0.0 | 0.1 |
| TLR4 | U88880.1 | −1.7 | 1.2 | 1.2 | 1.0 | 1.1 | | −3.9 | −1.1 | 1.3 | 1.4 | −2.8 | −0.1 |
| TNF | NM_000594.1 | 1.1 | −1.4 | 1.1 | 1.0 | −1.1 | −18.4 | −1.7 | −1.7 | 1.0 | 1.3 | 0.0 | −0.3 |
| TNFAIP3 | NM_006290.2 | 1.1 | 1.0 | 1.3 | 1.0 | 1.0 | | 1.2 | 1.4 | −1.4 | 1.5 | −0.2 | −2.9 |
| TNFRSF10A | NM_003844.2 | −1.3 | −1.6 | 1.1 | −1.2 | 1.0 | | 3.2 | 2.6 | 2.3 | 2.3 | 0.6 | 0.0 |
| TNFRSF10B | AF016266.1 | 1.0 | 1.1 | −1.3 | −1.1 | 1.2 | | 1.1 | −1.1 | 1.2 | 1.3 | 2.2 | −0.1 |
| TNFRSF11A | NM_003839.1 | 1.0 | 1.0 | 1.0 | 1.0 | −1.3 | | 1.3 | 1.3 | 1.3 | 1.0 | −0.1 | 0.2 |
| TNFRSF1A | NM_001065.2 | 1.0 | −1.1 | 1.3 | 1.0 | 1.1 | | −1.0 | 1.2 | −1.3 | −1.6 | −2.3 | 0.2 |
| TNFRSF1B | NM_001066.1 | 1.0 | −1.0 | −1.0 | 1.0 | 1.1 | | 1.2 | 1.4 | 2.7 | 2.7 | −0.2 | 0.1 |
| TNFRSF7 | NM_001242.1 | | | | | | | −1.1 | 1.1 | 1.8 | 1.8 | −2.2 | −0.1 |
| TNFRSF8 | NM_001243.2 | 1.2 | 1.0 | 1.2 | −1.4 | 1.1 | | −1.1 | 1.0 | −1.5 | −1.1 | −2.1 | −0.4 |
| TNFRSF9 | NM_001561.4 | −1.4 | −1.9 | −1.4 | 1.6 | 1.0 | | −1.3 | 1.4 | 1.0 | −1.0 | −2.7 | 2.0 |
| TNFSF10 | NM_003810.2 | 1.1 | 1.0 | 1.3 | −1.1 | 1.1 | | −4.8 | −1.7 | −1.8 | −2.0 | −3.1 | 0.2 |
| TNFSF11 | NM_003701.2 | −1.1 | −1.1 | 1.4 | 1.2 | −1.0 | | −1.5 | 1.1 | 1.2 | 2.0 | −2.6 | −0.8 |
| TNFSF14 | AF064090.1 | 1.0 | 1.0 | 1.3 | 1.0 | 1.0 | | 1.1 | −1.6 | −1.3 | −2.9 | 2.6 | 1.5 |
| TNFSF4 | NM_003326.2 | 1.0 | 1.5 | 2.3 | 1.7 | 1.3 | | −3.2 | −3.8 | −1.1 | 1.0 | 0.6 | −2.1 |
| TNFSF7 | NM_001252.2 | | | | | | | −3.2 | −3.8 | −1.1 | 1.0 | 0.6 | −2.1 |
| TNFSF8 | NM_001244.2 | 1.2 | 1.0 | 1.2 | −1.4 | 1.1 | | −2.1 | −1.7 | 1.5 | 1.0 | −0.4 | 0.4 |
| TRADD | NM_003789.2 | 1.1 | −1.0 | 1.0 | 1.6 | 1.1 | | −1.1 | −1.1 | −1.2 | −1.2 | 0.0 | 0.0 |
| TRAF1 | NM_005658.3 | −1.2 | −1.1 | 1.0 | −2.1 | 1.2 | | −1.2 | 1.1 | −1.1 | 1.4 | −2.3 | −2.4 |
| TRAF2 | NM_021138.3 | −1.0 | −1.2 | 1.1 | −1.0 | 1.2 | | 1.5 | 1.6 | 1.2 | 1.2 | −0.2 | 0.0 |
| TRAF3 | U21092.1 | 1.1 | 1.0 | 1.0 | 1.1 | 1.2 | | −1.0 | 1.8 | 1.6 | 1.7 | −2.9 | −0.1 |
| TRAF6 | U78798.1 | −1.0 | 1.1 | −1.0 | −1.1 | 1.6 | | −1.3 | −1.7 | −1.8 | −1.7 | 0.3 | −0.1 |
| VCAM1 | M60335.1 | −1.0 | 1.2 | 1.4 | 1.8 | 1.0 | | 1.1 | −1.4 | −1.4 | −2.3 | 2.4 | 0.9 |
| VEGF | AF022375.1 | | | | | | | −2.2 | −1.8 | 1.0 | 1.0 | −0.5 | 0.0 |
| VEGFB | U43368.1 | −1.2 | 1.0 | 1.4 | 1.0 | 1.3 | | −1.1 | −1.1 | 1.3 | 1.2 | 0.0 | 0.1 |
| VEGFC | NM_005429.2 | −1.0 | −1.0 | −1.0 | −1.0 | 2.1 | | 1.3 | 1.3 | 2.3 | 2.2 | 0.0 | 0.1 |
| YWHAZ | M86400.1 | 1.0 | 1.0 | 1.1 | −1.2 | 1.1 | | −1.3 | −1.1 | −1.2 | −1.2 | −0.2 | 0.0 |

A: Idebenone Alone Expression
B: Idebenone + UVB Expression
C: Coffee cherry Alone Expression
D: Coffee cherry + UVB Expression
E: UVB Alone Expression
F: Green tea + UVB Expression
G: Coffee cherry + H2O2 Expression (24 hours)
H: H2O2 Alone Expression (24 hours)
I: Coffee cherry + H2O2 Expression (8 hours)
J: H2O2 Alone Expression (8 hours)
K: Difference between Coffee cherry + H2o2 and H202 Alone Expression (24 hours)
L: Difference between Coffee cherry + H2o2 and H202 Alone Expression (8 hours)

DATA TABLE 14
Mitochondrial/Cellular respiration/Mitochondrial biogenesis genes

| Official | | | | | | | | | | | | |
|---|---|---|---|---|---|---|---|---|---|---|---|---|
| Gene | | | | | | | | | | | | |
| Symbol | GenBank | A | B | C | D | E | R | G | H | I | J | K |
| ACTB | NM_001101 | 1.0 | 1.1 | 1.1 | 1.0 | 0.0 | 0.0 | 1.0 | −1.1 | −1.2 | 1.4 | −1.0 |
| AIFM2 | NM_032797 | 1.0 | 1.4 | −1.5 | −1.6 | −0.4 | 0.1 | 1.0 | 1.2 | 1.0 | 1.0 | 1.2 |
| AIP | NM_003977 | −1.4 | −1.2 | −1.3 | −1.3 | −0.2 | 0.0 | −1.1 | −1.2 | 1.2 | 1.1 | −1.1 |
| B2M | NM_004048 | −1.1 | 1.8 | 1.2 | 1.0 | −2.9 | 0.2 | −1.0 | 1.3 | −1.4 | 1.0 | −1.0 |
| BAK1 | NM_001188 | 2.3 | 1.9 | 1.7 | 1.5 | 0.4 | 0.2 | −1.1 | −1.3 | 1.3 | 1.0 | −1.0 |
| BBC3 | NM_014417 | 1.0 | 1.2 | −1.2 | −1.2 | −0.1 | 0.0 | −1.2 | 1.3 | 1.4 | 1.2 | 1.0 |
| BCL2 | NM_000633 | −1.3 | 1.0 | 1.8 | 1.8 | −2.3 | 0.0 | 1.2 | 1.6 | 1.3 | 1.0 | 1.1 |
| BCL2L1 | NM_138578 | −1.7 | 1.2 | 1.1 | −1.2 | −2.9 | 2.3 | 1.0 | 1.0 | 1.0 | −1.1 | 2.7 |
| BID | NM_001196 | −1.4 | 1.1 | 1.5 | 1.4 | −2.6 | 0.1 | −1.1 | 1.3 | 1.0 | 1.0 | 1.1 |
| BNIP3 | NM_004052 | 1.1 | −1.2 | 1.0 | 1.5 | 2.3 | −0.5 | 1.0 | 1.1 | −1.2 | −1.4 | 1.3 |
| CDKN2A | NM_000077 | −2.2 | −1.1 | −1.1 | −1.1 | −1.1 | 0.0 | 1.0 | −1.0 | −1.6 | 1.0 | 1.2 |
| COX10 | NM_001303 | 1.1 | 1.6 | 1.1 | 1.3 | −0.5 | −0.3 | 1.1 | 1.1 | 1.0 | −1.1 | 1.0 |
| COX18 | NM_173827 | −1.4 | −1.3 | −1.3 | −1.3 | 0.0 | 0.0 | −1.0 | 1.3 | 1.0 | 1.0 | 1.0 |
| CPT1B | NM_004377 | −1.4 | 1.1 | −1.0 | −1.2 | −2.6 | 0.2 | −1.0 | −1.7 | −1.1 | −1.4 | 1.0 |
| CPT2 | NM_000098 | −1.2 | −1.3 | −1.2 | −1.2 | 0.1 | 0.1 | −1.1 | −1.2 | −1.1 | 1.0 | −1.0 |
| DNAJC19 | NM_145261 | −1.3 | −1.0 | −1.4 | −1.2 | −0.2 | −0.2 | 1.1 | 1.2 | −1.2 | −1.5 | −1.0 |
| DNM1L | NM_005690 | −3.8 | 1.2 | 1.1 | 1.1 | −4.9 | 0.0 | −1.6 | 1.3 | 1.0 | 1.0 | −1.1 |
| FIS1 | NM_016068 | −1.2 | 1.1 | −2.5 | −1.9 | −2.3 | −0.7 | 1.0 | −1.3 | 2.9 | −1.1 | −1.1 |
| FXC1 | NM_012192 | −1.3 | −1.5 | 1.3 | 1.1 | 0.2 | 0.2 | 1.2 | 1.1 | 1.0 | 1.0 | −1.2 |
| GAPDH | NM_002046 | −1.0 | 1.0 | −1.0 | −1.0 | −2.0 | 0.0 | −1.1 | −1.4 | −1.5 | 1.1 | 1.1 |
| GRPEL1 | NM_025196 | 2.4 | 2.4 | 1.7 | 1.8 | 0.1 | 0.0 | −2.6 | 1.2 | 1.2 | −1.0 | 1.2 |
| HGDC | SA_00105 | 1.2 | 2.8 | −1.7 | 1.2 | −1.6 | −2.9 | 1.2 | 1.1 | 1.6 | 1.0 | −1.6 |
| HPRT1 | NM_000194 | −2.1 | −2.0 | −1.4 | −1.3 | −0.1 | −0.2 | 1.2 | 1.3 | 1.0 | 1.2 | −1.7 |
| HSP90AA1 | NM_001017963 | 1.1 | 1.2 | 1.1 | 1.2 | −0.1 | 0.0 | 1.0 | 1.0 | 1.0 | 1.0 | 1.3 |
| HSPD1 | NM_002156 | −1.3 | 3.1 | 2.1 | 2.0 | −4.3 | 0.1 | 1.1 | 1.1 | 1.2 | 1.2 | 1.1 |
| IMMP1L | NM_144981 | 1.9 | 2.2 | −1.2 | 1.1 | −0.3 | −2.2 | −1.0 | 1.2 | 1.1 | 1.0 | −1.2 |
| IMMP2L | NM_032549 | −2.1 | −1.2 | −1.3 | −1.2 | −1.0 | −0.1 | 1.5 | −1.1 | −1.1 | −1.3 | 1.3 |
| LRPPRC | NM_133259 | −2.1 | −1.7 | −1.5 | −1.3 | −0.3 | −0.2 | 1.0 | 1.0 | −1.0 | −1.1 | −1.4 |
| MFN1 | NM_033540 | −1.4 | −1.1 | −1.0 | −1.1 | −0.3 | 0.1 | −1.0 | 1.1 | 1.0 | 1.0 | −1.0 |
| MFN2 | NM_014874 | 1.1 | −1.3 | 1.4 | 1.3 | 2.4 | 0.0 | −1.0 | 1.1 | 1.0 | 1.0 | −1.1 |
| MIPEP | NM_005932 | 1.1 | 1.2 | 1.3 | 1.1 | −0.1 | 0.2 | −2.1 | −1.2 | −1.2 | 1.0 | 1.1 |
| MSTO1 | NM_018116 | 2.7 | 2.5 | 1.7 | 1.6 | 0.2 | 0.2 | 1.0 | −1.4 | 1.3 | 1.0 | 1.2 |
| MTX2 | NM_006554 | 1.0 | 1.2 | −1.3 | −1.1 | −0.2 | −0.2 | −1.0 | 1.0 | −1.2 | −1.1 | −1.1 |
| NEFL | NM_006158 | −1.9 | 1.6 | 1.6 | −1.1 | −3.5 | 2.6 | −1.1 | 1.0 | 1.0 | 1.2 | 1.9 |
| OPA1 | NM_130837 | −2.2 | 1.4 | 1.9 | 1.7 | −3.6 | 0.2 | 1.0 | 1.1 | 1.1 | −1.0 | 1.3 |
| PMAIP1 | NM_021127 | 4.3 | 4.0 | 3.4 | 3.4 | 0.3 | 0.0 | −1.2 | −1.2 | 1.3 | 1.4 | 1.2 |
| RHOT1 | NM_018307 | −1.3 | −1.2 | −1.2 | −1.1 | −0.1 | −0.1 | −1.2 | −1.9 | 1.1 | −1.1 | −1.1 |
| RHOT2 | NM_138769 | −1.0 | 1.5 | 1.4 | 1.7 | −2.5 | −0.2 | −1.4 | −1.3 | 1.2 | 1.0 | 1.3 |
| RPL13A | NM_012423 | −1.2 | −1.0 | −1.0 | −1.0 | −0.1 | 0.0 | 1.1 | −1.1 | 1.0 | −1.4 | 1.1 |
| SFN | NM_006142 | 3.4 | 1.7 | 1.4 | 1.5 | 1.7 | −0.1 | −1.1 | 1.0 | 1.3 | 1.0 | −2.8 |
| SH3GLB1 | NM_016009 | −1.9 | 1.2 | −1.1 | −1.0 | −3.1 | 0.0 | 1.0 | −1.0 | −1.1 | −1.1 | 1.6 |
| SLC25A1 | NM_005984 | −1.4 | −1.4 | 1.1 | 1.0 | 0.0 | 0.0 | −1.4 | −1.1 | 1.2 | −1.1 | 1.6 |
| SLC25A10 | NM_012140 | 2.2 | 2.2 | 1.5 | 1.6 | 0.0 | −0.1 | −1.1 | −1.3 | 1.3 | 1.0 | 4.3 |
| SLC25A12 | NM_003705 | −1.2 | −1.2 | 1.2 | 1.1 | 0.0 | 0.1 | −1.1 | 1.2 | 1.0 | −1.1 | −1.1 |
| SLC25A13 | NM_014251 | 1.0 | 2.0 | 1.9 | 1.6 | −1.0 | 0.2 | −1.1 | 1.0 | −1.2 | −1.5 | 1.0 |
| SLC25A14 | NM_003951 | 1.1 | 1.3 | −1.1 | −1.0 | −0.2 | 0.0 | 1.9 | 1.0 | 1.1 | 1.5 | −1.7 |
| SLC25A15 | NM_014252 | 1.4 | 1.7 | 1.5 | 1.2 | −0.3 | 0.3 | 1.1 | 1.1 | 1.1 | −1.0 | −1.0 |
| SLC25A16 | NM_152707 | −1.4 | −1.1 | −1.2 | −1.1 | −0.2 | −0.2 | −1.1 | 1.2 | −1.0 | 1.0 | −1.1 |
| SLC25A17 | NM_006358 | 1.4 | 1.4 | 1.4 | 1.3 | 0.0 | 0.1 | 1.1 | −1.0 | −1.2 | −1.2 | −1.2 |
| SLC25A19 | NM_021734 | 1.7 | 1.5 | 1.3 | 1.3 | 0.1 | 0.0 | 1.2 | −1.2 | 1.1 | 2.0 | 1.5 |
| SLC25A2 | NM_031947 | −1.2 | 1.0 | 1.7 | 1.0 | −2.2 | 0.6 | −1.0 | −1.5 | 1.2 | 1.0 | 1.1 |
| SLC25A20 | NM_000387 | −1.1 | 1.0 | −1.1 | −1.2 | −2.2 | 0.1 | 1.0 | −1.1 | 1.3 | −1.6 | 1.7 |
| SLC25A21 | NM_030631 | −1.7 | −1.7 | −1.3 | 2.8 | 0.0 | −4.1 | 1.0 | 1.0 | −1.3 | 1.3 | 1.1 |
| SLC25A22 | NM_024698 | 2.2 | 1.9 | 2.1 | 2.2 | 0.2 | −0.1 | −1.0 | 1.2 | 1.1 | −1.5 | 1.6 |
| SLC25A23 | NM_024103 | −3.2 | −3.9 | 1.1 | −1.1 | 0.7 | 2.1 | −1.2 | 1.0 | 1.0 | 1.1 | 1.8 |
| SLC25A24 | NM_013386 | −1.1 | −1.0 | 1.2 | 1.2 | −0.1 | 0.0 | −1.0 | 1.3 | 2.1 | 1.7 | 1.1 |
| SLC25A25 | NM_052901 | 3.2 | 3.2 | 2.5 | 2.6 | 0.1 | −0.1 | 1.0 | 1.2 | 1.0 | −1.2 | −1.0 |
| SLC25A27 | NM_004277 | −3.6 | 1.3 | 2.0 | 1.2 | −4.9 | 0.7 | 1.1 | 1.4 | 1.0 | −1.2 | 1.2 |
| SLC25A3 | NM_002635 | −1.1 | −1.1 | −1.1 | −1.1 | 0.0 | 0.0 | −1.2 | 1.1 | −1.1 | 1.0 | 1.3 |
| SLC25A30 | NM_001010875 | 1.3 | 1.3 | 1.1 | 1.1 | 0.0 | 0.0 | −1.6 | −1.1 | −1.2 | 1.0 | 1.2 |
| SLC25A31 | NM_031291 | 1.3 | −1.1 | 1.4 | 1.5 | 2.5 | −0.2 | −1.2 | 1.0 | 1.5 | 1.0 | −1.1 |
| SLC25A37 | NM_016612 | −3.0 | −1.6 | 1.7 | 2.0 | −1.4 | −0.3 | −1.0 | −1.6 | −1.3 | −1.3 | −1.0 |
| SLC25A4 | NM_001151 | −1.1 | 1.1 | 1.2 | 1.0 | −2.2 | 0.1 | 1.2 | −1.3 | −1.2 | 1.0 | 1.1 |
| SLC25A5 | NM_001152 | −1.9 | −1.8 | −1.1 | −1.2 | −0.2 | 0.1 | 1.0 | 1.1 | 1.3 | 1.7 | 9.1 |
| SOD1 | NM_000454 | −1.0 | −1.0 | −1.2 | −1.1 | 0.0 | 0.0 | −1.0 | −1.1 | −1.1 | −1.0 | −1.3 |
| SOD2 | NM_000636 | 2.7 | 2.3 | 1.6 | 1.5 | 0.5 | 0.1 | −1.2 | 1.0 | −1.1 | 1.5 | 1.1 |
| STARD3 | NM_006804 | 1.2 | 1.1 | 1.1 | 1.2 | 0.0 | 0.0 | 1.0 | −1.9 | −1.1 | 1.1 | 1.2 |
| TAZ | NM_000116 | −2.0 | 1.1 | 1.0 | 1.4 | −3.1 | −0.4 | −1.1 | −1.4 | 1.0 | −2.4 | 1.0 |
| TIMM10 | NM_012456 | 1.9 | 1.9 | 1.0 | 1.1 | 0.0 | −0.1 | 1.2 | 1.1 | 1.0 | 1.0 | 2.7 |
| TIMM17A | NM_006335 | 1.3 | 1.5 | 1.1 | 1.3 | −0.3 | −0.2 | −1.1 | 1.1 | 1.0 | −1.3 | −1.1 |
| TIMM17B | NM_005834 | 1.2 | −1.0 | −1.1 | 1.3 | 2.2 | −2.3 | 1.0 | −1.5 | −1.0 | −1.0 | 1.2 |
| TIMM22 | NM_013337 | 1.3 | 1.3 | 1.0 | −1.0 | 0.0 | 2.1 | 1.1 | −1.1 | 1.3 | 1.4 | 1.0 |
| TIMM23 | NM_006327 | 1.3 | 1.4 | 1.2 | 1.2 | −0.1 | 0.0 | 1.1 | −1.2 | −1.0 | −1.3 | −1.1 |
| TIMM44 | NM_006351 | 1.2 | 1.1 | −1.0 | −1.1 | 0.1 | 0.0 | −1.2 | −1.2 | −1.4 | −1.0 | −1.0 |
| TIMM50 | NM_001001563 | 1.4 | 1.9 | 1.2 | 1.1 | −0.6 | 0.0 | 1.0 | −1.0 | −1.1 | 1.4 | −1.1 |
| TIMM8A | NM_004085 | 1.9 | 1.9 | 1.6 | 1.9 | 0.0 | −0.3 | 1.0 | 1.0 | 1.1 | −1.1 | 1.1 |
| TIMM8B | NM_012459 | 1.5 | 1.4 | −1.1 | −1.1 | 0.1 | 0.0 | −1.2 | 1.1 | 1.0 | −1.4 | 1.0 |
| TIMM9 | NM_012460 | −1.1 | 1.1 | −1.0 | 1.1 | −2.1 | −2.2 | 2.2 | 1.2 | −1.0 | 1.2 | 1.4 |
| TOMM20 | NM_014765 | −1.1 | −1.1 | −1.1 | −1.1 | 0.0 | 0.0 | 1.0 | 1.0 | −1.9 | −1.4 | 1.2 |
| TOMM22 | NM_020243 | −1.1 | 1.1 | 1.1 | −1.1 | −2.2 | 2.2 | 1.0 | −1.1 | −1.1 | −1.3 | −1.2 |
| TOMM34 | NM_006809 | 1.9 | 2.0 | 1.0 | 1.1 | −0.1 | −0.1 | 1.2 | −1.1 | 1.0 | 1.0 | 1.0 |
| TOMM40 | NM_006114 | 2.4 | 2.2 | 1.6 | 1.9 | 0.2 | −0.2 | 1.0 | −1.8 | 1.2 | 1.0 | 1.3 |
| TOMM40L | NM_032174 | 1.6 | 1.3 | 1.5 | 1.3 | 0.2 | 0.1 | 1.0 | −1.0 | −1.1 | −1.1 | 1.0 |
| TOMM70A | NM_014820 | −1.0 | −1.2 | −2.1 | 1.2 | 0.2 | −3.2 | 1.1 | −1.3 | 1.1 | −1.2 | 1.0 |
| TP53 | NM_000546 | −3.1 | −2.0 | −1.5 | −1.5 | −1.1 | 0.0 | −1.1 | −1.3 | −1.0 | 1.2 | 1.2 |
| TSPO | NM_000714 | 1.0 | −1.1 | −1.1 | −1.0 | 2.1 | 0.0 | 1.0 | −1.1 | 1.1 | 1.2 | 1.1 |
| UCP1 | NM_021833 | 1.1 | 1.2 | 1.4 | 1.1 | −0.1 | 0.3 | 1.1 | 1.3 | 1.2 | 1.4 | 1.1 |
| UCP2 | NM_003355 | 1.0 | −1.2 | −1.1 | −1.1 | 2.2 | 0.1 | 1.0 | −1.1 | 1.0 | 1.0 | 1.2 |
| UCP3 | NM_003356 | −1.1 | −1.2 | −1.1 | −1.3 | 0.1 | 0.2 | −1.1 | 1.1 | 1.1 | 1.4 | 1.0 |
| UXT | NM_004182 | 1.3 | 1.1 | −1.2 | −1.1 | 0.1 | −0.1 | 1.0 | −1.1 | 1.5 | 1.3 | 1.2 |

A: Coffee cherry + H2O2 Expression (24 hours)
B: H2O2 Alone Expression (24 hours)
C: Coffee cherry + H2O2 Expression (8 hours)
D: H2O2 Alone Expression (8 hours)
E: Difference between Coffee cherry + H2O2 and H2O2 Alone Expression (24 hours)
F: Difference between Coffee cherry + H2O2 and H2O2 Alone Expression (8 hours)
G: Idebenone Alone Expression
H: Idebenone + UVB Expression
I: CoffeeBerry Alone Expression
J: CoffeeBerry + UVB Expression
K: UVB Alone Expression

DATA TABLE 15
Nitric Oxide Synthase genes

| Official Gene | | | | | | | | | | | |
|---|---|---|---|---|---|---|---|---|---|---|---|
| Symbol | A | B | C | D | E | F | G | H | I | J | K |
| AKT1 | 1.6 | 1.4 | 1.8 | 1.6 | 0.2 | 0.2 | 1.0 | −1.6 | −1.2 | −1.2 | −1.0 |
| ALOX12 | 1.1 | 2.2 | −1.3 | −1.9 | −1.1 | 0.6 | −1.1 | 1.8 | 1.0 | 1.0 | 1.0 |
| APOE | −1.1 | 1.1 | −1.3 | −1.0 | −2.2 | −0.3 | 1.0 | −1.2 | 1.0 | 1.2 | 1.1 |
| ARG2 | 1.9 | 1.6 | 1.1 | −1.2 | 0.3 | 2.2 | 1.0 | 1.2 | −1.4 | −1.0 | 1.1 |
| ATOX1 | 1.6 | 1.5 | 1.1 | 1.1 | 0.1 | −0.1 | −1.0 | −1.0 | 1.4 | 1.5 | 1.0 |
| CAMK1 | 1.1 | 1.1 | 1.8 | 1.6 | 0.0 | 0.2 | −1.1 | −1.7 | 1.2 | 1.1 | −1.3 |
| CAT | −1.9 | −1.8 | −1.7 | −1.4 | −0.2 | −0.3 | 1.2 | −1.3 | 1.4 | −1.1 | 1.2 |
| CCNA1 | 1.2 | 2.7 | −1.0 | 1.2 | −1.5 | −2.3 | 1.0 | 1.0 | −1.1 | −1.3 | 1.2 |
| CCS | −1.3 | −1.3 | −1.2 | −1.1 | 0.0 | −0.2 | −1.0 | −1.2 | −1.6 | 1.1 | −2.2 |
| CDKN1A | 1.3 | 1.2 | −1.2 | −1.1 | 0.2 | −0.1 | 1.4 | −1.1 | −1.2 | −1.2 | −1.2 |
| CYBA | −1.0 | −1.1 | 1.1 | 1.0 | 0.1 | 0.0 | 1.0 | −1.2 | 1.1 | −1.3 | 1.0 |
| CYGB | 1.2 | 1.1 | 1.1 | 1.1 | 0.2 | 0.0 | 1.2 | −1.4 | 1.2 | 1.0 | 1.1 |
| DDAH2 | −2.8 | −2.5 | −2.9 | −2.9 | −0.2 | 0.0 | 1.1 | 1.0 | 1.0 | 4.4 | −1.7 |
| DLG4 | −1.8 | −1.7 | −1.5 | −2.3 | −0.1 | 0.9 | 1.0 | −1.0 | −1.2 | −1.0 | 1.3 |
| DNCL1 | −1.1 | −1.9 | −1.1 | 1.5 | 0.8 | −2.6 | 1.0 | 1.0 | −1.0 | −1.1 | 1.3 |
| DND1 | 1.6 | −3.5 | 1.2 | 1.1 | 5.1 | 0.2 | 1.0 | −1.0 | −1.2 | −1.3 | 1.1 |
| DUOX1 | −1.1 | −1.6 | −1.1 | 2.2 | 0.5 | −3.2 | 1.0 | 1.2 | 1.2 | 1.4 | 2.0 |
| DUOX2 | 2.2 | 2.5 | 1.8 | 1.5 | −0.2 | 0.3 | −1.1 | 2.0 | −1.2 | −1.5 | 1.2 |
| DUSP1 | 4.3 | 3.3 | 3.4 | 2.9 | 1.0 | 0.5 | −1.4 | 1.2 | 2.6 | 1.0 | −1.0 |
| EGFR | −2.6 | −4.0 | −1.0 | 1.1 | 1.4 | −2.1 | 1.8 | −1.0 | 1.0 | −1.0 | −1.2 |
| EPX | −1.1 | 1.3 | 1.2 | 1.0 | −2.4 | 0.2 | −1.6 | −2.2 | −1.3 | 1.2 | 1.0 |
| FOXM1 | −1.3 | 1.0 | −1.1 | −1.2 | −2.3 | 0.1 | −1.0 | −1.5 | 1.3 | −1.6 | −1.1 |
| GCH1 | 2.4 | 2.2 | 1.6 | 1.4 | 0.3 | 0.2 | 1.0 | −1.7 | 1.6 | 1.9 | 1.5 |
| GCHFR | −1.1 | −1.1 | −1.5 | −1.4 | 0.1 | −0.2 | 1.0 | 1.0 | 1.0 | 1.0 | −1.1 |
| GLA | 2.3 | 2.1 | −1.0 | −1.0 | 0.2 | 0.0 | −1.2 | 1.0 | 1.0 | −1.1 | −1.3 |
| GLRX2 | 1.5 | 1.6 | 1.5 | 1.6 | −0.1 | −0.2 | 1.1 | 1.0 | 1.3 | 1.0 | −1.0 |
| GPR156 | 1.3 | 1.1 | 1.8 | 1.4 | 0.2 | 0.4 | 1.3 | 1.3 | 1.0 | 1.0 | −1.4 |
| GPX1 | −1.3 | −1.3 | −1.1 | −1.1 | 0.0 | 0.0 | 1.0 | −1.1 | −1.1 | 1.1 | 2.0 |
| GPX2 | 1.2 | 1.2 | 1.0 | −1.2 | 0.0 | 2.2 | 1.0 | −1.2 | −1.1 | 1.0 | 1.2 |
| GPX3 | 1.1 | 1.0 | −1.1 | −1.0 | 0.1 | −0.1 | 1.0 | −1.2 | 1.1 | −1.2 | 1.4 |
| GPX4 | −1.4 | −1.4 | −1.3 | −1.3 | 0.0 | 0.0 | −1.3 | −1.1 | 1.1 | 1.0 | −1.6 |
| GPX5 | 1.1 | −1.3 | −1.6 | −1.3 | 2.4 | −0.3 | 1.0 | 1.4 | −1.1 | −1.2 | −1.0 |
| GRIN2D | −1.1 | −1.0 | 1.6 | −1.0 | −0.1 | 2.6 | 1.0 | 1.1 | −1.2 | 1.2 | −1.1 |
| GSS | 1.1 | 1.2 | −1.0 | −1.0 | 0.0 | 0.0 | −1.1 | −1.1 | 1.2 | 1.2 | −1.2 |
| HSP90AB1 | 1.1 | 1.0 | 1.0 | 1.1 | 0.0 | −0.1 | 1.0 | −1.3 | −1.2 | 1.2 | −1.6 |
| IL10 | 3.0 | 2.2 | 1.1 | 1.2 | 0.8 | 0.0 | 1.1 | 1.1 | 1.2 | 1.0 | −1.5 |
| IL8 | NA | NA | NA | NA | NA | NA | −1.2 | 1.0 | 1.0 | 1.0 | 1.1 |
| INS | −1.1 | 1.2 | −1.1 | 1.6 | −2.3 | −2.7 | −1.0 | 1.1 | 1.2 | 1.0 | 1.0 |
| JUN | 1.4 | 1.9 | 1.2 | 1.8 | −0.5 | −0.7 | 1.0 | −1.1 | −1.1 | −1.6 | 1.2 |
| KRT1 | 3.4 | 2.7 | −1.4 | 1.1 | 0.7 | −2.6 | 1.1 | 1.0 | −1.1 | −1.1 | −1.0 |
| LPO | 1.3 | −1.7 | −1.3 | −2.2 | 2.9 | 0.9 | 1.1 | 1.1 | 1.5 | 1.1 | −1.0 |
| MBL2 | −1.4 | −1.0 | −1.4 | 1.2 | −0.4 | −2.6 | 1.0 | 1.0 | −1.1 | 1.0 | 1.3 |
| MPO | 1.1 | −1.1 | 1.9 | 1.3 | 2.2 | 0.6 | 1.0 | 1.0 | −1.3 | −1.2 | 1.2 |
| MSRA | −1.4 | −1.3 | 1.5 | 1.4 | −0.1 | 0.1 | 1.0 | 1.2 | 1.4 | 1.4 | 1.2 |
| MT3 | −1.3 | −1.6 | 1.3 | 1.1 | 0.3 | 0.2 | −1.1 | 1.1 | 1.2 | −1.0 | 1.3 |
| MTL5 | −1.1 | 1.0 | 1.0 | 1.1 | −2.1 | −0.1 | 1.5 | −1.1 | 1.5 | −1.1 | 1.1 |
| MYB | 1.2 | −1.1 | 1.6 | 1.5 | 2.3 | 0.1 | 1.0 | 1.0 | 1.1 | −1.1 | 1.0 |
| NCF1 | 1.4 | 1.4 | −1.6 | 1.0 | 0.0 | −2.7 | −1.7 | −1.2 | −1.5 | −1.1 | 1.1 |
| NCF2 | 6.5 | 4.9 | 1.8 | 2.0 | 1.6 | −0.2 | 1.1 | 1.6 | −1.1 | 1.0 | 1.0 |
| NME5 | −2.7 | −3.0 | −1.2 | −1.8 | 0.3 | 0.5 | 1.0 | 1.2 | 1.7 | 1.0 | −1.4 |
| NOS1 | −1.0 | −1.2 | −1.3 | 1.1 | 0.2 | −2.4 | 1.9 | 1.2 | 1.1 | 1.1 | 1.2 |
| NOS2A | −1.1 | −1.1 | −1.1 | 1.1 | 0.0 | −2.2 | 1.0 | 1.1 | 1.0 | −1.6 | −1.1 |
| NOS3 | −1.7 | −2.6 | 1 | 1.1 | 0.9 | −0.1 | 1.0 | −1.0 | 1.0 | 1.2 | −1.0 |
| NOSIP | −1.0 | −1.1 | 1.4 | 1.3 | 0.1 | 0.1 | 1.0 | −1.2 | 1.0 | −1.3 | −1.4 |
| NOSTRIN | 1.2 | 1.5 | 1.4 | 1.4 | −0.3 | 0.0 | 1.1 | 1.0 | −1.4 | −1.5 | 1.0 |
| NOX5 | 1.4 | 1.1 | 1.3 | 1.1 | 0.4 | 0.2 | 1.0 | −1.2 | 1.2 | 1.1 | 1.2 |
| NQO1 | −1.0 | −1.1 | −1.5 | −1.5 | 0.0 | 0.0 | −1.3 | −1.2 | 1.1 | −1.4 | −1.0 |
| NRF1 | −1.2 | −1.2 | 1.2 | 1.3 | 0.0 | −0.1 | −1.0 | −1.4 | −1.1 | −1.1 | 1.0 |
| NUDT1 | 1.4 | 1.2 | −1.3 | −1.3 | 0.2 | 0.0 | 1.3 | −1.1 | −1.0 | 1.0 | −1.1 |
| OXR1 | −1.4 | 1.2 | −1.2 | −1.3 | −2.5 | 0.0 | 1.0 | 1.1 | −1.3 | −1.0 | −1.0 |
| PGC | −1.2 | 1.2 | −1.1 | −1.1 | −2.4 | 0.0 | −1.2 | 1.2 | 1.2 | 1.0 | 1.2 |
| PKAR1B | −2.4 | −1.7 | 1.7 | 2.3 | −0.7 | −0.7 | 1.1 | −1.0 | 1.1 | −1.0 | −1.0 |
| PNKP | 1.3 | 1.2 | 1.2 | 1.2 | 0.0 | 0.0 | 1.0 | −1.3 | −1.0 | 1.6 | −1.0 |
| PP3CA | −1.1 | −1.2 | −1.3 | −1.4 | 0.2 | 0.0 | 1.0 | 1.1 | 1.2 | 1.1 | 1.3 |
| PRDX2 | 1.0 | 1.1 | −1.0 | −1.1 | −0.1 | 0.1 | −1.3 | 1.0 | 1.1 | 1.0 | −1.0 |
| PRDX6 | 1.1 | 1.0 | 1.1 | 1.1 | 0.0 | 0.0 | −1.1 | 1.1 | 1.0 | 1.0 | −1.1 |
| PREX1 | −1.8 | 1.4 | 1.7 | 2.2 | −3.2 | −0.6 | 1.0 | −1.1 | 1.2 | 1.0 | −1.1 |
| PRG3 | −2.2 | −1.8 | 1.6 | 1.4 | −0.4 | 0.2 | 1.0 | −1.0 | 1.1 | 1.0 | 2.3 |
| PRKCA | 1.2 | −1.0 | −1.0 | −1.1 | 2.2 | 0.1 | 1.0 | −1.1 | 2.1 | 1.5 | 1.0 |
| PRNP | −1.3 | −1.1 | −1.5 | −1.6 | −0.2 | 0.0 | −1.0 | 1.0 | 1.3 | 1.1 | 1.0 |
| RNF7 | 3.2 | 4.2 | −1.0 | 1.1 | −1.0 | −2.1 | 1.2 | −1.0 | 1.4 | 1.0 | 1.0 |
| SCARA3 | −1.5 | −1.4 | −1.1 | −1.1 | −0.1 | 0.0 | 1.1 | −1.0 | −1.2 | 1.0 | 1.3 |
| SCRT2 | NA | NA | NA | NA | NA | NA | 1.4 | 1.3 | −1.4 | 1.0 | 1.1 |
| SELS | 1.4 | 1.5 | 1.3 | 1.3 | 0.0 | 0.0 | 1.0 | 1.0 | 1.3 | 1.6 | 1.7 |
| SEPP1 | −2.8 | −3.4 | −1.4 | −1.1 | 0.6 | −0.3 | 1.2 | 1.0 | 1.0 | 1.0 | 1.1 |
| SGK2 | −1.3 | −1.5 | 1.0 | 1.4 | 0.2 | −0.4 | 1.2 | −1.2 | −1.0 | −1.3 | −1.0 |
| SIRT2 | −1.1 | −1.0 | 1.1 | 1.1 | −0.1 | 0.0 | −1.0 | −1.1 | −1.3 | 1.0 | −1.1 |
| SOD1 | −1.0 | −1.0 | −1.2 | −1.1 | 0.0 | 0.0 | −1.0 | −1.1 | −1.1 | −1.0 | −1.3 |
| SOD2 | 2.8 | 2.3 | 1.6 | 1.5 | 0.5 | 0.1 | −1.2 | 1.0 | −1.1 | 1.5 | 1.1 |
| SOD3 | −1.1 | 1.0 | 1.1 | 1.1 | −2.2 | −0.1 | −1.3 | 1.1 | −1.1 | −1.4 | −1.1 |
| SRXN1 | 3.7 | 2.4 | 1.1 | 1.1 | 1.2 | 0.0 | 1.5 | −1.1 | 1.0 | 1.1 | 1.5 |
| TFAM | 1.2 | 1.4 | −2.1 | −1.8 | −0.2 | −0.3 | −1.2 | 1.1 | −1.2 | −1.6 | 1.1 |
| TPO | −2.4 | −2.0 | −1.1 | −1.7 | −0.4 | 0.7 | 1.0 | 1.0 | −1.8 | 1.0 | 1.1 |
| TROAP | 3.0 | 3.9 | −2.0 | −1.5 | −0.9 | −0.5 | −1.0 | −1.1 | −2.4 | 1.1 | 1.2 |
| TTN | −1.0 | −1.0 | −2.3 | 1.0 | 0.0 | −3.3 | 1.0 | 1.3 | 1.0 | 1.5 | −2.2 |
| TXNRD2 | −1.2 | −1.2 | 1.1 | 1.0 | 0.1 | 0.1 | −1.3 | −1.6 | −1.2 | −1.4 | −1.1 |
| UNR | NA | NA | NA | NA | NA | NA | 1.0 | 1.4 | 1.6 | 1.0 | 1.7 |
| VEGFA | 1.3 | 1.4 | 1.3 | 1.5 | −0.1 | −0.1 | −1.7 | −1.3 | 1.2 | −1.1 | 1.2 |

A: Coffee cherry + H2O2 Expression (24 hours)
B: H2O2 Alone Expression (24 hours)
C: Coffee cherry + H2O2 Expression (8 hours)
D: H2O2 Alone Expression (8 hours)
E: Difference between Coffee cherry + H2o2 and H202 Alone Expression (24 hours)
F: Difference between Coffee cherry + H2o2 and H202 Alone Expression (8 hours)
G: Idebenone Alone Expression
H: Idebenone + UVB Expression
I: CoffeeBerry Alone Expression
J: CoffeeBerry + UVB Expression
K: UVB Alone Expression

DATA TABLE 7
Statiscally significant Gene (p-value <0.05) with a fold change greater than 1.8 or less than −1.8

| A | B | C | | E | F |
|---|---|---|---|---|---|
| Sequence | coffee cherry + | coffee cherry + | D | coffee cherry + | Idebenone + |
| name | H2O2 24 hrs | H2O2 8 hrs | coffee cherry | UVB | UVB |
| 1553575_at | 10.9 | 4.7 | n.s. | n.s. | n.s. |
| 1554007_at | n.s. | −3.0 | n.s. | n.s. | n.s. |
| 1554948_at | n.s. | 4.9 | n.s. | n.s. | n.s. |
| 1555846_a_at | n.s. | −2.1 | n.s. | n.s. | n.s. |
| 1555875_at | −1.9 | n.s. | n.s. | n.s. | n.s. |
| 1556097_at | −2.9 | −2.8 | n.s. | n.s. | n.s. |
| 1556216_s_at | −2.3 | −2.2 | n.s. | n.s. | n.s. |
| 1556242_a_at | −2.2 | −1.8 | n.s. | n.s. | n.s. |
| 1556332_at | 1.9 | n.s. | n.s. | n.s. | n.s. |
| 1556545_at | 14.0 | 7.4 | n.s. | n.s. | n.s. |
| 1556936_at | −3.1 | n.s. | n.s. | n.s. | n.s. |
| 1557118_a_at | n.s. | −2.2 | n.s. | n.s. | n.s. |
| 1557286_at | −3.7 | −2.0 | n.s. | n.s. | n.s. |
| 1557287_at | 4.5 | n.s. | n.s. | n.s. | n.s. |
| 1557302_at | n.s. | −1.9 | n.s. | n.s. | n.s. |
| 1557341_x_at | −2.6 | n.s. | n.s. | n.s. | n.s. |
| 1557348_at | −3.4 | n.s. | n.s. | n.s. | n.s. |
| 1557383_a_at | n.s. | −5.3 | n.s. | n.s. | n.s. |
| 1557667_at | −2.1 | −1.9 | n.s. | n.s. | n.s. |
| 1557740_a_at | −8.4 | n.s. | n.s. | n.s. | n.s. |
| 1558105_a_at | −2.2 | n.s. | n.s. | n.s. | n.s. |
| 1558236_at | −2.6 | n.s. | n.s. | n.s. | n.s. |
| 1558237_x_at | −3.1 | n.s. | n.s. | n.s. | n.s. |
| 1558250_s_at | −3.3 | −3.0 | n.s. | n.s. | n.s. |
| 1558401_at | n.s. | −2.0 | n.s. | n.s. | n.s. |
| 1558445_at | −4.5 | −2.6 | n.s. | n.s. | n.s. |
| 1558515_at | n.s. | 2.4 | n.s. | n.s. | n.s. |
| 1558604_a_at | −3.0 | −3.3 | n.s. | n.s. | n.s. |
| 1558605_at | −2.4 | −2.8 | n.s. | n.s. | n.s. |
| 1558750_a_at | n.s. | −3.2 | n.s. | n.s. | n.s. |
| 1558801_at | −2.5 | −2.4 | n.s. | n.s. | n.s. |
| 1558836_at | n.s. | 3.4 | n.s. | n.s. | n.s. |
| 1558837_a_at | n.s. | 2.0 | n.s. | n.s. | n.s. |
| 1558890_at | n.s. | −3.9 | n.s. | n.s. | n.s. |
| 1558906_a_at | n.s. | −3.4 | n.s. | n.s. | n.s. |
| 1558920_at | 7.1 | n.s. | n.s. | n.s. | n.s. |
| 1559229_at | n.s. | 5.1 | n.s. | n.s. | n.s. |
| 1559867_at | −3.8 | n.s. | n.s. | n.s. | n.s. |
| 1560071_a_at | n.s. | 2.0 | n.s. | n.s. | n.s. |
| 1560208_at | 2.6 | 2.2 | n.s. | n.s. | n.s. |
| 1560579_s_at | n.s. | 2.3 | n.s. | n.s. | n.s. |
| 1561064_a_at | n.s. | −2.0 | n.s. | n.s. | n.s. |
| 1562012_at | −3.2 | −2.7 | n.s. | n.s. | n.s. |
| 1562013_a_at | −2.7 | −2.0 | n.s. | n.s. | n.s. |
| 1562056_at | n.s. | 2.3 | n.s. | n.s. | n.s. |
| 1562098_at | 2.3 | n.s. | n.s. | n.s. | n.s. |
| 1562777_at | n.s. | −4.3 | n.s. | n.s. | n.s. |
| 1563012_x_at | n.s. | 2.0 | n.s. | n.s. | n.s. |
| 1563414_at | n.s. | 2.5 | n.s. | n.s. | n.s. |
| 1565495_at | n.s. | −2.5 | n.s. | n.s. | n.s. |
| 1565577_s_at | −2.2 | n.s. | n.s. | n.s. | n.s. |
| 1565783_at | 2.8 | 5.8 | n.s. | n.s. | n.s. |
| 1568633_a_at | −3.3 | n.s. | n.s. | n.s. | n.s. |
| 1569129_s_at | −2.0 | n.s. | n.s. | n.s. | n.s. |
| 1569765_at | 1.9 | 2.2 | n.s. | n.s. | n.s. |
| 1570061_at | 4.0 | n.s. | n.s. | n.s. | n.s. |
| 1570100_at | 3.4 | n.s. | n.s. | n.s. | n.s. |
| 182-FIP | −1.8 | −2.1 | n.s. | n.s. | n.s. |
| 208187_s_at | n.s. | 4.9 | n.s. | n.s. | n.s. |
| 210230_at | 4.8 | 8.4 | n.s. | n.s. | n.s. |
| 213156_at | −2.7 | −3.5 | n.s. | n.s. | n.s. |
| 213158_at | −2.5 | −3.0 | n.s. | n.s. | n.s. |
| 213567_at | −2.1 | n.s. | n.s. | n.s. | n.s. |
| 213817_at | −4.0 | −1.9 | n.s. | n.s. | n.s. |
| 213832_at | −2.0 | n.s. | n.s. | n.s. | n.s. |
| 214202_at | −1.9 | −2.3 | n.s. | n.s. | n.s. |
| 214808_at | −2.2 | −2.3 | n.s. | n.s. | n.s. |
| 214862_x_at | −5.7 | −3.0 | n.s. | n.s. | n.s. |
| 214967_at | n.s. | 4.5 | n.s. | n.s. | n.s. |
| 215128_at | n.s. | −1.8 | n.s. | n.s. | n.s. |
| 215287_at | n.s. | −1.8 | n.s. | n.s. | n.s. |
| 217166_at | 2.0 | n.s. | n.s. | n.s. | n.s. |
| 217536_x_at | n.s. | 2.1 | n.s. | n.s. | n.s. |
| 217540_at | −2.3 | −2.8 | n.s. | n.s. | n.s. |
| 217554_at | −3.2 | −2.5 | n.s. | n.s. | n.s. |
| 217604_at | n.s. | −2.2 | n.s. | n.s. | n.s. |
| 220494_s_at | −2.9 | −5.3 | n.s. | n.s. | n.s. |
| 220726_at | n.s. | 4.6 | n.s. | n.s. | n.s. |
| 221200_at | 7.4 | n.s. | n.s. | n.s. | n.s. |
| 222184_at | −2.9 | −1.9 | n.s. | n.s. | n.s. |
| 222284_at | n.s. | 3.0 | n.s. | n.s. | n.s. |
| 224769_at | n.s. | −2.3 | n.s. | n.s. | n.s. |
| 224778_s_at | n.s. | −1.8 | n.s. | n.s. | n.s. |
| 224811_at | −2.6 | n.s. | n.s. | n.s. | n.s. |
| 225256_at | −1.8 | −2.0 | n.s. | n.s. | n.s. |
| 225356_at | −2.3 | n.s. | n.s. | n.s. | n.s. |
| 225725_at | −2.5 | −2.0 | n.s. | n.s. | n.s. |
| 225893_at | n.s. | −1.9 | n.s. | n.s. | n.s. |
| 225917_at | −2.2 | n.s. | n.s. | n.s. | n.s. |
| 226203_at | −2.3 | −1.8 | n.s. | n.s. | n.s. |
| 226250_at | −2.6 | −2.3 | n.s. | n.s. | n.s. |
| 226282_at | −3.0 | −2.1 | n.s. | n.s. | n.s. |
| 226316_at | −2.5 | −1.8 | n.s. | n.s. | n.s. |
| 226362_at | −2.3 | −2.5 | n.s. | n.s. | n.s. |
| 226365_at | −3.3 | −3.0 | n.s. | n.s. | n.s. |
| 226392_at | −1.9 | n.s. | n.s. | n.s. | n.s. |
| 226457_at | n.s. | −1.9 | n.s. | n.s. | n.s. |
| 226458_at | n.s. | −2.2 | n.s. | n.s. | n.s. |
| 226520_at | n.s. | −2.0 | n.s. | n.s. | n.s. |
| 226532_at | −1.8 | n.s. | n.s. | n.s. | n.s. |
| 226542_at | −2.3 | n.s. | n.s. | n.s. | n.s. |
| 226546_at | n.s. | −2.0 | n.s. | n.s. | n.s. |
| 226550_at | −2.0 | n.s. | n.s. | n.s. | n.s. |
| 226773_at | n.s. | −2.2 | n.s. | n.s. | n.s. |
| 226885_at | −1.8 | n.s. | n.s. | n.s. | n.s. |
| 226964_at | n.s. | −2.2 | n.s. | n.s. | n.s. |
| 227041_at | −2.1 | n.s. | n.s. | n.s. | n.s. |
| 227044_at | 3.3 | n.s. | n.s. | n.s. | n.s. |
| 227051_at | −2.4 | n.s. | n.s. | n.s. | n.s. |
| 227061_at | −2.2 | n.s. | n.s. | n.s. | n.s. |
| 227082_at | −3.0 | −4.2 | n.s. | n.s. | n.s. |
| 227121_at | −2.2 | −3.3 | n.s. | n.s. | n.s. |
| 227126_at | −3.2 | n.s. | n.s. | n.s. | n.s. |
| 227184_at | 3.0 | n.s. | n.s. | n.s. | n.s. |
| 227193_at | n.s. | −2.4 | n.s. | n.s. | n.s. |
| 227221_at | −2.3 | −1.8 | n.s. | n.s. | n.s. |
| 227252_at | −2.5 | n.s. | n.s. | n.s. | n.s. |
| 227283_at | 2.0 | n.s. | n.s. | n.s. | n.s. |
| 227306_at | 2.2 | n.s. | n.s. | n.s. | n.s. |
| 227422_at | −2.1 | −1.9 | n.s. | n.s. | n.s. |
| 227503_at | −2.1 | n.s. | n.s. | n.s. | n.s. |
| 227531_at | n.s. | −1.8 | n.s. | n.s. | n.s. |
| 227533_at | −3.6 | −1.8 | n.s. | n.s. | n.s. |
| 227565_at | −3.2 | −2.5 | n.s. | n.s. | n.s. |
| 227623_at | −1.9 | n.s. | n.s. | n.s. | n.s. |
| 227655_at | −2.3 | n.s. | n.s. | n.s. | n.s. |
| 227663_at | −2.4 | −2.3 | n.s. | n.s. | n.s. |
| 227682_at | −2.7 | n.s. | n.s. | n.s. | n.s. |
| 227929_at | −1.8 | −1.9 | n.s. | n.s. | n.s. |
| 227955_s_at | −3.8 | n.s. | n.s. | n.s. | n.s. |
| 228032_s_at | −2.8 | −2.1 | n.s. | n.s. | n.s. |
| 228045_at | −3.6 | −2.0 | n.s. | n.s. | n.s. |
| 228049_x_at | n.s. | −2.0 | n.s. | n.s. | n.s. |
| 228084_at | −3.1 | −2.7 | n.s. | n.s. | n.s. |
| 228156_at | −2.3 | n.s. | n.s. | n.s. | n.s. |
| 228159_at | n.s. | 2.5 | n.s. | n.s. | n.s. |
| 228216_at | −2.3 | −2.3 | n.s. | n.s. | n.s. |
| 228242_at | −2.6 | −2.0 | n.s. | n.s. | n.s. |
| 228304_at | −2.4 | −3.7 | n.s. | n.s. | n.s. |
| 228315_at | −2.1 | −2.4 | n.s. | n.s. | n.s. |
| 228333_at | −4.6 | −2.1 | n.s. | n.s. | n.s. |
| 228346_at | n.s. | −2.9 | n.s. | n.s. | n.s. |
| 228390_at | −6.6 | −6.6 | n.s. | n.s. | n.s. |
| 228478_at | −2.3 | n.s. | n.s. | n.s. | n.s. |
| 228528_at | n.s. | −3.1 | n.s. | n.s. | n.s. |
| 228694_at | −2.5 | n.s. | n.s. | n.s. | n.s. |
| 228740_at | −2.7 | −2.7 | n.s. | n.s. | n.s. |
| 228742_at | −4.6 | −3.4 | n.s. | n.s. | n.s. |
| 228750_at | −11.5 | n.s. | n.s. | n.s. | n.s. |
| 228773_at | −1.9 | n.s. | n.s. | n.s. | n.s. |
| 228781_at | −2.1 | n.s. | n.s. | n.s. | n.s. |
| 228811_at | −3.7 | n.s. | n.s. | n.s. | n.s. |
| 228812_at | n.s. | −2.5 | n.s. | n.s. | n.s. |
| 228850_s_at | −2.4 | n.s. | n.s. | n.s. | n.s. |
| 228955_at | 2.4 | n.s. | n.s. | n.s. | n.s. |
| 228963_at | −2.5 | −2.1 | n.s. | n.s. | n.s. |
| 229024_at | −5.9 | −2.6 | n.s. | n.s. | n.s. |
| 229072_at | −6.3 | −7.9 | n.s. | n.s. | n.s. |
| 229121_at | −9.1 | n.s. | n.s. | n.s. | n.s. |
| 229189_s_at | −2.2 | −1.9 | n.s. | n.s. | n.s. |
| 229190_at | −2.2 | −2.0 | n.s. | n.s. | n.s. |
| 229281_at | −3.8 | n.s. | n.s. | n.s. | n.s. |
| 229297_at | n.s. | −2.9 | n.s. | n.s. | n.s. |
| 229315_at | −3.5 | n.s. | n.s. | n.s. | n.s. |
| 229319_at | −1.9 | n.s. | n.s. | n.s. | n.s. |
| 229333_at | −1.9 | n.s. | n.s. | n.s. | n.s. |
| 229359_at | −2.7 | −2.5 | n.s. | n.s. | n.s. |
| 229384_at | −2.4 | −2.5 | n.s. | n.s. | n.s. |
| 229460_at | n.s. | −2.9 | n.s. | n.s. | n.s. |
| 229479_at | −2.2 | n.s. | n.s. | n.s. | n.s. |
| 229512_at | −1.9 | n.s. | n.s. | n.s. | n.s. |
| 229569_at | −10.3 | −10.1 | n.s. | n.s. | n.s. |
| 229572_at | −2.3 | n.s. | n.s. | n.s. | n.s. |
| 229602_at | −4.6 | n.s. | n.s. | n.s. | n.s. |
| 229615_at | −4.3 | −3.8 | n.s. | n.s. | n.s. |
| 229641_at | −2.9 | n.s. | n.s. | n.s. | n.s. |
| 229699_at | −2.4 | −3.8 | n.s. | n.s. | n.s. |
| 229705_at | −2.5 | n.s. | n.s. | n.s. | n.s. |
| 229710_at | −2.2 | n.s. | n.s. | n.s. | n.s. |
| 229756_at | n.s. | −2.5 | n.s. | n.s. | n.s. |
| 229757_at | −2.1 | n.s. | n.s. | n.s. | n.s. |
| 229795_at | −4.8 | −5.8 | n.s. | n.s. | n.s. |
| 229810_at | −2.2 | −2.0 | n.s. | n.s. | n.s. |
| 229815_at | n.s. | −4.1 | n.s. | n.s. | n.s. |
| 229948_at | −1.9 | −2.0 | n.s. | n.s. | n.s. |
| 229994_at | −2.7 | n.s. | n.s. | n.s. | n.s. |
| 230003_at | −7.0 | −2.0 | n.s. | n.s. | n.s. |
| 230090_at | 5.4 | 3.4 | n.s. | n.s. | n.s. |
| 230127_at | −4.2 | −3.1 | n.s. | n.s. | n.s. |
| 230183_at | −3.0 | n.s. | n.s. | n.s. | n.s. |
| 230211_at | −4.7 | −2.3 | n.s. | n.s. | n.s. |
| 230227_at | n.s. | −3.2 | n.s. | n.s. | n.s. |
| 230240_at | 2.4 | n.s. | n.s. | n.s. | n.s. |
| 230304_at | 2.1 | n.s. | n.s. | n.s. | n.s. |
| 230345_at | 5.5 | 3.6 | n.s. | n.s. | n.s. |
| 230383_x_at | −2.1 | −1.9 | n.s. | n.s. | n.s. |
| 230406_at | −2.9 | n.s. | n.s. | n.s. | n.s. |
| 230407_at | n.s. | −2.5 | n.s. | n.s. | n.s. |
| 230431_at | −2.3 | n.s. | n.s. | n.s. | n.s. |
| 230446_at | −7.7 | n.s. | n.s. | n.s. | n.s. |
| 230449_x_at | n.s. | −2.7 | n.s. | n.s. | n.s. |
| 230483_at | n.s. | −2.3 | n.s. | n.s. | n.s. |
| 230503_at | n.s. | 1.8 | n.s. | n.s. | n.s. |
| 230683_at | −3.7 | n.s. | n.s. | n.s. | n.s. |
| 230741_at | n.s. | −2.8 | n.s. | n.s. | n.s. |
| 230766_at | 1.9 | 1.8 | n.s. | n.s. | n.s. |
| 230773_at | −2.0 | n.s. | n.s. | n.s. | n.s. |
| 230860_at | n.s. | −1.9 | n.s. | n.s. | n.s. |
| 230927_at | −2.3 | −2.0 | n.s. | n.s. | n.s. |
| 230968_at | 2.2 | −2.8 | n.s. | n.s. | n.s. |
| 231055_at | n.s. | −1.9 | n.s. | n.s. | n.s. |
| 231069_at | −2.1 | n.s. | n.s. | n.s. | n.s. |
| 231193_s_at | −2.6 | −2.2 | n.s. | n.s. | n.s. |
| 231238_at | n.s. | −2.7 | n.s. | n.s. | n.s. |
| 231576_at | −2.9 | −4.8 | n.s. | n.s. | n.s. |
| 231597_x_at | 2.7 | n.s. | n.s. | n.s. | n.s. |
| 231890_at | n.s. | −2.2 | n.s. | n.s. | n.s. |
| 231963_at | −8.5 | −3.3 | n.s. | n.s. | n.s. |
| 231993_at | −2.1 | n.s. | n.s. | n.s. | n.s. |
| 232088_x_at | n.s. | −3.2 | n.s. | n.s. | n.s. |
| 232125_at | −2.8 | −1.8 | n.s. | n.s. | n.s. |
| 232156_at | −1.8 | n.s. | n.s. | n.s. | n.s. |
| 232484_at | −2.1 | n.s. | n.s. | n.s. | n.s. |
| 232535_at | −3.1 | n.s. | n.s. | n.s. | n.s. |
| 232538_at | −2.0 | n.s. | n.s. | n.s. | n.s. |
| 232656_at | −7.0 | n.s. | n.s. | n.s. | n.s. |
| 232795_at | −2.6 | n.s. | n.s. | n.s. | n.s. |
| 232903_at | n.s. | 6.5 | n.s. | n.s. | n.s. |
| 233105_at | n.s. | 2.2 | n.s. | n.s. | n.s. |
| 233335_at | −4.3 | −2.8 | n.s. | n.s. | n.s. |
| 233354_at | n.s. | −1.9 | n.s. | n.s. | n.s. |
| 233376_at | 2.7 | 7.1 | n.s. | n.s. | n.s. |
| 233485_at | −3.1 | n.s. | n.s. | n.s. | n.s. |
| 233518_at | −1.9 | n.s. | n.s. | n.s. | n.s. |
| 233723_at | 3.5 | 3.2 | n.s. | n.s. | n.s. |
| 233814_at | −5.5 | n.s. | n.s. | n.s. | n.s. |
| 234340_at | −3.3 | n.s. | n.s. | n.s. | n.s. |
| 234578_at | 2.1 | n.s. | n.s. | n.s. | n.s. |
| 234723_x_at | 2.9 | n.s. | n.s. | n.s. | n.s. |
| 234983_at | 1.8 | n.s. | n.s. | n.s. | n.s. |
| 235000_at | −2.1 | n.s. | n.s. | n.s. | n.s. |
| 235028_at | n.s. | 2.9 | n.s. | n.s. | n.s. |
| 235046_at | −4.3 | n.s. | n.s. | n.s. | n.s. |
| 235072_s_at | n.s. | −1.8 | n.s. | n.s. | n.s. |
| 235078_at | −1.8 | n.s. | n.s. | n.s. | n.s. |
| 235123_at | −2.0 | n.s. | n.s. | n.s. | n.s. |
| 235124_at | n.s. | −2.1 | n.s. | n.s. | n.s. |
| 235171_at | −8.6 | −2.7 | n.s. | n.s. | n.s. |
| 235207_at | 2.0 | n.s. | n.s. | n.s. | n.s. |
| 235224_s_at | −12.7 | n.s. | n.s. | n.s. | n.s. |
| 235227_at | −5.8 | −2.9 | n.s. | n.s. | n.s. |
| 235264_at | −1.9 | n.s. | n.s. | n.s. | n.s. |
| 235279_at | n.s. | −1.9 | n.s. | n.s. | n.s. |
| 235299_at | −2.5 | −2.1 | n.s. | n.s. | n.s. |
| 235302_at | n.s. | −3.1 | n.s. | n.s. | n.s. |
| 235304_at | −2.3 | −3.7 | n.s. | n.s. | n.s. |
| 235352_at | −3.0 | −3.1 | n.s. | n.s. | n.s. |
| 235407_at | 1.9 | n.s. | n.s. | n.s. | n.s. |
| 235427_at | −6.4 | −4.5 | n.s. | n.s. | n.s. |
| 235428_at | n.s. | −2.9 | n.s. | n.s. | n.s. |
| 235434_at | n.s. | −2.3 | n.s. | n.s. | n.s. |
| 235438_at | −3.0 | n.s. | n.s. | n.s. | n.s. |
| 235459_at | −3.2 | n.s. | n.s. | n.s. | n.s. |
| 235532_at | n.s. | −2.5 | n.s. | n.s. | n.s. |
| 235556_at | n.s. | −2.5 | n.s. | n.s. | n.s. |
| 235571_at | −2.3 | −5.0 | n.s. | n.s. | n.s. |
| 235581_at | −2.1 | −1.9 | n.s. | n.s. | n.s. |
| 235585_at | −2.1 | −1.9 | n.s. | n.s. | n.s. |
| 235612_at | −1.8 | −1.8 | n.s. | n.s. | n.s. |
| 235655_at | −8.8 | n.s. | n.s. | n.s. | n.s. |
| 235658_at | −2.2 | n.s. | n.s. | n.s. | n.s. |
| 235696_at | −2.0 | n.s. | n.s. | n.s. | n.s. |
| 235733_at | −3.7 | n.s. | n.s. | n.s. | n.s. |
| 235761_at | n.s. | 2.0 | n.s. | n.s. | n.s. |
| 235764_at | −3.9 | −1.9 | n.s. | n.s. | n.s. |
| 235830_at | −6.2 | n.s. | n.s. | n.s. | n.s. |
| 235831_at | −2.9 | n.s. | n.s. | n.s. | n.s. |
| 235889_at | −2.7 | −2.2 | n.s. | n.s. | n.s. |
| 235890_at | −4.0 | −2.0 | n.s. | n.s. | n.s. |
| 235919_at | −1.9 | −2.1 | n.s. | n.s. | n.s. |
| 235931_at | −2.4 | −2.6 | n.s. | n.s. | n.s. |
| 235938_at | −2.6 | −2.9 | n.s. | n.s. | n.s. |
| 236004_at | −1.9 | −2.5 | n.s. | n.s. | n.s. |
| 236038_at | −12.2 | −6.0 | n.s. | n.s. | n.s. |
| 236089_at | −6.5 | −3.4 | n.s. | n.s. | n.s. |
| 236097_at | 2.0 | n.s. | n.s. | n.s. | n.s. |
| 236105_at | −4.1 | n.s. | n.s. | n.s. | n.s. |
| 236174_at | n.s. | −6.1 | n.s. | n.s. | n.s. |
| 236180_at | −2.3 | n.s. | n.s. | n.s. | n.s. |
| 236194_at | −2.1 | −2.5 | n.s. | n.s. | n.s. |
| 236196_at | n.s. | −3.1 | n.s. | n.s. | n.s. |
| 236335_at | −4.7 | n.s. | n.s. | n.s. | n.s. |
| 236344_at | −2.3 | n.s. | n.s. | n.s. | n.s. |
| 236350_at | −2.3 | −2.7 | n.s. | n.s. | n.s. |
| 236364_at | −2.7 | −1.9 | n.s. | n.s. | n.s. |
| 236423_at | 8.9 | n.s. | n.s. | n.s. | n.s. |
| 236433_at | −2.3 | n.s. | n.s. | n.s. | n.s. |
| 236452_at | 3.3 | n.s. | n.s. | n.s. | n.s. |
| 236619_at | n.s. | −2.7 | n.s. | n.s. | n.s. |
| 236685_at | n.s. | 3.2 | n.s. | n.s. | n.s. |
| 236787_at | 2.0 | n.s. | n.s. | n.s. | n.s. |
| 236856_x_at | −1.9 | n.s. | n.s. | n.s. | n.s. |
| 236875_at | 7.6 | 2.8 | n.s. | n.s. | n.s. |
| 236898_at | 37.2 | 7.8 | n.s. | n.s. | n.s. |
| 236922_at | −1.8 | n.s. | n.s. | n.s. | n.s. |
| 236996_at | −3.2 | n.s. | n.s. | n.s. | n.s. |
| 237071_at | −2.3 | n.s. | n.s. | n.s. | n.s. |
| 237212_at | −2.6 | n.s. | n.s. | n.s. | n.s. |
| 237290_at | n.s. | 5.0 | n.s. | n.s. | n.s. |
| 237315_at | n.s. | −3.8 | n.s. | n.s. | n.s. |
| 237416_at | 2.7 | 2.5 | n.s. | n.s. | n.s. |
| 237435_at | 1.8 | n.s. | n.s. | n.s. | n.s. |
| 237622_at | −2.9 | n.s. | n.s. | n.s. | n.s. |
| 237768_x_at | n.s. | 1.8 | n.s. | n.s. | n.s. |
| 238030_at | n.s. | −2.6 | n.s. | n.s. | n.s. |
| 238050_at | −2.0 | n.s. | n.s. | n.s. | n.s. |
| 238109_at | −2.8 | n.s. | n.s. | n.s. | n.s. |
| 238178_at | −4.2 | −1.9 | n.s. | n.s. | n.s. |
| 238250_at | 5.8 | 3.5 | n.s. | n.s. | n.s. |
| 238308_at | 4.0 | 3.9 | n.s. | n.s. | n.s. |
| 238360_s_at | n.s. | 3.0 | n.s. | n.s. | n.s. |
| 238389_s_at | 1.9 | n.s. | n.s. | n.s. | n.s. |
| 238431_at | −1.9 | n.s. | n.s. | n.s. | n.s. |
| 238456_at | −2.0 | −2.4 | n.s. | n.s. | n.s. |
| 238463_at | −6.1 | n.s. | n.s. | n.s. | n.s. |
| 238483_at | −2.1 | −1.8 | n.s. | n.s. | n.s. |
| 238501_at | −1.9 | −2.4 | n.s. | n.s. | n.s. |
| 238557_at | 2.6 | n.s. | n.s. | n.s. | n.s. |
| 238559_at | 2.0 | n.s. | n.s. | n.s. | n.s. |
| 238565_at | n.s. | −2.3 | n.s. | n.s. | n.s. |
| 238573_at | n.s. | −2.2 | n.s. | n.s. | n.s. |
| 238576_at | −2.6 | −2.0 | n.s. | n.s. | n.s. |
| 238604_at | −3.9 | n.s. | n.s. | n.s. | n.s. |
| 238617_at | −4.8 | n.s. | n.s. | n.s. | n.s. |
| 238620_at | n.s. | −2.7 | n.s. | n.s. | n.s. |
| 238673_at | −5.3 | n.s. | n.s. | n.s. | n.s. |
| 238684_at | −3.0 | n.s. | n.s. | n.s. | n.s. |
| 238712_at | 3.5 | 3.1 | n.s. | n.s. | n.s. |
| 238733_at | n.s. | −3.1 | n.s. | n.s. | n.s. |
| 238766_at | 3.0 | n.s. | n.s. | n.s. | n.s. |
| 238782_at | −3.1 | n.s. | n.s. | n.s. | n.s. |
| 238824_at | −2.1 | −1.8 | n.s. | n.s. | n.s. |
| 238861_at | −4.3 | −3.2 | n.s. | n.s. | n.s. |
| 238890_at | −2.5 | −3.6 | n.s. | n.s. | n.s. |
| 238932_at | −4.6 | −4.0 | n.s. | n.s. | n.s. |
| 238934_at | n.s. | −2.3 | n.s. | n.s. | n.s. |
| 239066_at | n.s. | −3.2 | n.s. | n.s. | n.s. |
| 239218_at | −2.6 | n.s. | n.s. | n.s. | n.s. |
| 239231_at | n.s. | −2.0 | n.s. | n.s. | n.s. |
| 239266_at | n.s. | 9.1 | n.s. | n.s. | n.s. |
| 239278_at | −2.1 | n.s. | n.s. | n.s. | n.s. |
| 239331_at | 2.0 | 5.7 | n.s. | n.s. | n.s. |
| 239370_at | −3.1 | −2.4 | n.s. | n.s. | n.s. |
| 239503_at | −4.2 | −3.4 | n.s. | n.s. | n.s. |
| 239543_s_at | −3.4 | n.s. | n.s. | n.s. | n.s. |
| 239669_at | 16.7 | n.s. | n.s. | n.s. | n.s. |
| 239708_at | −3.6 | −4.7 | n.s. | n.s. | n.s. |
| 239710_at | −2.9 | −2.4 | n.s. | n.s. | n.s. |
| 239842_x_at | n.s. | 2.4 | n.s. | n.s. | n.s. |
| 239845_at | 3.2 | 2.2 | n.s. | n.s. | n.s. |
| 239847_at | −5.2 | n.s. | n.s. | n.s. | n.s. |
| 239866_at | −1.9 | −2.1 | n.s. | n.s. | n.s. |
| 239951_at | −2.3 | n.s. | n.s. | n.s. | n.s. |
| 239973_at | −3.0 | n.s. | n.s. | n.s. | n.s. |
| 240020_at | n.s. | −2.1 | n.s. | n.s. | n.s. |
| 240095_at | −2.3 | n.s. | n.s. | n.s. | n.s. |
| 240128_at | n.s. | 3.0 | n.s. | n.s. | n.s. |
| 240165_at | n.s. | 3.1 | n.s. | n.s. | n.s. |
| 240190_at | 2.5 | n.s. | n.s. | n.s. | n.s. |
| 240219_at | −3.0 | −2.6 | n.s. | n.s. | n.s. |
| 240233_at | 2.1 | n.s. | n.s. | n.s. | n.s. |
| 240366_at | 3.8 | n.s. | n.s. | n.s. | n.s. |
| 240418_at | −3.0 | n.s. | n.s. | n.s. | n.s. |
| 240523_at | −3.1 | n.s. | n.s. | n.s. | n.s. |
| 240549_at | −1.8 | n.s. | n.s. | n.s. | n.s. |
| 240557_at | −2.5 | −2.8 | n.s. | n.s. | n.s. |
| 240885_at | 4.8 | n.s. | n.s. | n.s. | n.s. |
| 241114_s_at | −2.4 | n.s. | n.s. | n.s. | n.s. |
| 241263_at | 3.4 | n.s. | n.s. | n.s. | n.s. |
| 241359_at | −1.8 | −1.9 | n.s. | n.s. | n.s. |
| 241484_x_at | −2.4 | −1.8 | n.s. | n.s. | n.s. |
| 241689_at | n.s. | −1.8 | n.s. | n.s. | n.s. |
| 241721_at | 1.9 | −2.2 | n.s. | n.s. | n.s. |
| 241722_x_at | 2.0 | 3.8 | n.s. | n.s. | n.s. |
| 241815_at | n.s. | −2.1 | n.s. | n.s. | n.s. |
| 241823_at | n.s. | −2.1 | n.s. | n.s. | n.s. |
| 241838_at | n.s. | 2.8 | n.s. | n.s. | n.s. |
| 241863_x_at | −2.3 | −2.1 | n.s. | n.s. | n.s. |
| 241879_at | −2.9 | n.s. | n.s. | n.s. | n.s. |
| 241887_at | n.s. | −1.9 | n.s. | n.s. | n.s. |
| 241925_x_at | −9.1 | −2.0 | n.s. | n.s. | n.s. |
| 241936_x_at | n.s. | 2.5 | n.s. | n.s. | n.s. |
| 242005_at | 3.4 | n.s. | n.s. | n.s. | n.s. |
| 242051_at | −2.4 | n.s. | n.s. | n.s. | n.s. |
| 242107_x_at | −3.8 | n.s. | n.s. | n.s. | n.s. |
| 242134_at | n.s. | −4.1 | n.s. | n.s. | n.s. |
| 242289_at | −2.4 | n.s. | n.s. | n.s. | n.s. |
| 242312_x_at | −2.6 | −2.4 | n.s. | n.s. | n.s. |
| 242321_at | −2.6 | n.s. | n.s. | n.s. | n.s. |
| 242323_at | −1.8 | n.s. | n.s. | n.s. | n.s. |
| 242358_at | −2.2 | n.s. | n.s. | n.s. | n.s. |
| 242366_at | −2.0 | −2.0 | n.s. | n.s. | n.s. |
| 242376_at | n.s. | 1.9 | n.s. | n.s. | n.s. |
| 242421_at | 3.2 | n.s. | n.s. | n.s. | n.s. |
| 242471_at | n.s. | 2.3 | n.s. | n.s. | n.s. |
| 242486_at | 2.1 | n.s. | n.s. | n.s. | n.s. |
| 242523_at | n.s. | −2.3 | n.s. | n.s. | n.s. |
| 242606_at | n.s. | −1.9 | n.s. | n.s. | n.s. |
| 242719_at | 3.7 | 1.8 | n.s. | n.s. | n.s. |
| 242818_x_at | n.s. | −2.6 | n.s. | n.s. | n.s. |
| 242845_at | −6.1 | −2.6 | n.s. | n.s. | n.s. |
| 242979_at | −4.6 | −2.1 | n.s. | n.s. | n.s. |
| 243115_at | 3.8 | n.s. | n.s. | n.s. | n.s. |
| 243179_at | −2.6 | −2.0 | n.s. | n.s. | n.s. |
| 243278_at | −4.4 | n.s. | n.s. | n.s. | n.s. |
| 243302_at | −3.7 | n.s. | n.s. | n.s. | n.s. |
| 243366_s_at | −8.2 | n.s. | n.s. | n.s. | n.s. |
| 243404_at | 4.4 | 3.4 | n.s. | n.s. | n.s. |
| 243417_at | −2.4 | −2.6 | n.s. | n.s. | n.s. |
| 243489_at | 5.3 | 3.0 | n.s. | n.s. | n.s. |
| 243551_at | n.s. | 5.9 | n.s. | n.s. | n.s. |
| 243564_at | −2.6 | n.s. | n.s. | n.s. | n.s. |
| 243606_at | −2.7 | −3.1 | n.s. | n.s. | n.s. |
| 243641_at | n.s. | −2.8 | n.s. | n.s. | n.s. |
| 243671_at | n.s. | −2.3 | n.s. | n.s. | n.s. |
| 243680_at | −2.6 | n.s. | n.s. | n.s. | n.s. |
| 243697_at | −4.2 | n.s. | n.s. | n.s. | n.s. |
| 243707_at | −2.0 | −2.1 | n.s. | n.s. | n.s. |
| 243844_at | −13.3 | −3.4 | n.s. | n.s. | n.s. |
| 243907_at | −2.7 | n.s. | n.s. | n.s. | n.s. |
| 243925_at | n.s. | 5.5 | n.s. | n.s. | n.s. |
| 243934_at | n.s. | 2.5 | n.s. | n.s. | n.s. |
| 243947_s_at | 2.4 | n.s. | n.s. | n.s. | n.s. |
| 243976_at | −2.1 | n.s. | n.s. | n.s. | n.s. |
| 244007_at | −3.0 | n.s. | n.s. | n.s. | n.s. |
| 244025_at | 6.6 | 4.4 | n.s. | n.s. | n.s. |
| 244032_at | n.s. | −2.4 | n.s. | n.s. | n.s. |
| 244242_at | −2.0 | −2.2 | n.s. | n.s. | n.s. |
| 244271_at | −2.3 | −1.8 | n.s. | n.s. | n.s. |
| 244350_at | n.s. | 5.0 | n.s. | n.s. | n.s. |
| 244354_at | −2.2 | −2.4 | n.s. | n.s. | n.s. |
| 244533_at | −3.0 | n.s. | n.s. | n.s. | n.s. |
| 244663_at | n.s. | −2.5 | n.s. | n.s. | n.s. |
| 244677_at | 21.2 | n.s. | n.s. | n.s. | n.s. |
| 244701_at | 2.7 | n.s. | n.s. | n.s. | n.s. |
| 244779_at | −3.3 | n.s. | n.s. | n.s. | n.s. |
| 244853_at | −2.0 | n.s. | n.s. | n.s. | n.s. |
| 244855_at | 4.1 | n.s. | n.s. | n.s. | n.s. |
| 244864_at | 2.6 | n.s. | n.s. | n.s. | n.s. |
| 76P | −2.0 | n.s. | n.s. | n.s. | n.s. |
| A_23_P10605 | n.s. | n.s. | n.s. | n.s. | 3.0 |
| A_23_P113263 | n.s. | n.s. | n.s. | n.s. | −1.8 |
| A_23_P113762 | n.s. | n.s. | n.s. | n.s. | −1.8 |
| A_23_P13202 | n.s. | n.s. | n.s. | n.s. | 1.9 |
| A_23_P134405 | n.s. | n.s. | 5.2 | n.s. | n.s. |
| A_23_P170719 | n.s. | n.s. | n.s. | −2.4 | −2.5 |
| A_23_P205500 | n.s. | n.s. | −1.8 | n.s. | n.s. |
| A_23_P44053 | n.s. | n.s. | −2.1 | −2.4 | −3.0 |
| A_24_P136155 | n.s. | n.s. | n.s. | −1.8 | n.s. |
| A_24_P144054 | n.s. | n.s. | n.s. | 1.9 | n.s. |
| A_24_P195454 | n.s. | n.s. | −1.8 | n.s. | n.s. |
| A_24_P195621 | n.s. | n.s. | −2.1 | n.s. | n.s. |
| A_24_P229766 | n.s. | n.s. | −2.4 | −1.8 | n.s. |
| A_24_P247303 | n.s. | n.s. | 4.3 | n.s. | n.s. |
| A_24_P289973 | n.s. | n.s. | 2.4 | n.s. | 4.4 |
| A_24_P315674 | n.s. | n.s. | −2.3 | n.s. | n.s. |
| A_24_P3627 | n.s. | n.s. | n.s. | −2.8 | n.s. |
| A_24_P375360 | n.s. | n.s. | −1.8 | n.s. | n.s. |
| A_24_P384379 | n.s. | n.s. | 11.1 | n.s. | n.s. |
| A_24_P399341 | n.s. | n.s. | 2.4 | n.s. | 2.2 |
| A_24_P41483 | n.s. | n.s. | −2.0 | n.s. | n.s. |
| A_24_P524164 | n.s. | n.s. | n.s. | 3.2 | 3.0 |
| A_24_P607107 | n.s. | n.s. | −1.8 | n.s. | n.s. |
| A_24_P622375 | n.s. | n.s. | 3.3 | 2.5 | 3.6 |
| A_24_P626812 | n.s. | n.s. | −1.9 | n.s. | n.s. |
| A_24_P67268 | n.s. | n.s. | −1.8 | n.s. | n.s. |
| A_24_P682550 | n.s. | n.s. | n.s. | −2.2 | n.s. |
| A_24_P752362 | n.s. | n.s. | n.s. | −1.8 | n.s. |
| A_24_P7820 | n.s. | n.s. | n.s. | n.s. | 4.1 |
| A_24_P794833 | n.s. | n.s. | −1.9 | n.s. | n.s. |
| A_24_P799680 | n.s. | n.s. | 9.0 | 9.9 | 5.2 |
| A_24_P835943 | n.s. | n.s. | n.s. | −1.9 | n.s. |
| A_24_P84719 | n.s. | n.s. | n.s. | −1.8 | −2.0 |
| A_24_P84738 | n.s. | n.s. | n.s. | n.s. | 4.9 |
| A_24_P913855 | n.s. | n.s. | −4.2 | n.s. | n.s. |
| A_24_P919931 | n.s. | n.s. | n.s. | −2.1 | n.s. |
| A_24_P922430 | n.s. | n.s. | n.s. | −2.0 | n.s. |
| A_24_P928031 | n.s. | n.s. | −2.6 | n.s. | n.s. |
| A_24_P942151 | n.s. | n.s. | n.s. | n.s. | 3.5 |
| A_32_P111919 | n.s. | n.s. | −7.4 | n.s. | n.s. |
| A_32_P135790 | n.s. | n.s. | 1.9 | n.s. | 1.8 |
| A_32_P138933 | n.s. | n.s. | n.s. | −2.1 | n.s. |
| A_32_P149404 | n.s. | n.s. | n.s. | −2.0 | n.s. |
| A_32_P157622 | n.s. | n.s. | n.s. | n.s. | 3.5 |
| A_32_P190944 | n.s. | n.s. | n.s. | 1.9 | n.s. |
| A_32_P205522 | n.s. | n.s. | n.s. | n.s. | 2.0 |
| A_32_P220567 | n.s. | n.s. | n.s. | n.s. | −1.8 |
| A_32_P45087 | n.s. | n.s. | 1.8 | n.s. | 1.9 |
| A_32_P71456 | n.s. | n.s. | 2.7 | n.s. | n.s. |
| A_32_P9931 | n.s. | n.s. | 2.2 | n.s. | n.s. |
| A1BG | n.s. | n.s. | n.s. | 2.2 | n.s. |
| AA019203 | n.s. | n.s. | 2.5 | 2.4 | n.s. |
| AA043564 | n.s. | n.s. | n.s. | 1.9 | n.s. |
| AA085955 | n.s. | n.s. | −2.6 | n.s. | n.s. |
| AA344632 | n.s. | n.s. | n.s. | n.s. | 2.4 |
| AA451708 | n.s. | n.s. | 3.8 | n.s. | n.s. |
| AA581414 | n.s. | n.s. | 3.7 | n.s. | 2.6 |
| AA586832 | n.s. | n.s. | n.s. | 2.9 | n.s. |
| AA631975 | n.s. | n.s. | 2.0 | n.s. | 1.9 |
| AA725860 | n.s. | n.s. | n.s. | 2.3 | 1.9 |
| AA918648 | n.s. | n.s. | n.s. | n.s. | 3.9 |
| AADACL1 | 2.3 | 2.1 | n.s. | n.s. | n.s. |
| AARSD1 | 1.8 | n.s. | n.s. | n.s. | n.s. |
| AASS | −2.1 | n.s. | n.s. | n.s. | n.s. |
| AB011149 | n.s. | n.s. | n.s. | 2.4 | 2.6 |
| ABCA1 | 2.9 | −1.9 | n.s. | n.s. | n.s. |
| ABCA5 | −2.6 | n.s. | n.s. | n.s. | n.s. |
| ABCA6 | −5.5 | n.s. | n.s. | n.s. | n.s. |
| ABCB10 | n.s. | n.s. | n.s. | −2.5 | n.s. |
| ABCC3 | −3.0 | n.s. | n.s. | n.s. | n.s. |
| ABCC6 | −2.2 | −1.9 | n.s. | n.s. | n.s. |
| ABCE1 | n.s. | 2.1 | n.s. | n.s. | n.s. |
| ABCF2 | 5.3 | 2.4 | n.s. | n.s. | n.s. |
| ABHD11 | 1.9 | n.s. | n.s. | n.s. | n.s. |
| ABHD13 | n.s. | n.s. | n.s. | n.s. | 5.4 |
| ABHD2 | 3.8 | 2.4 | n.s. | n.s. | n.s. |
| ABHD3 | 2.4 | n.s. | n.s. | n.s. | n.s. |
| ABHD5 | 3.0 | 1.8 | n.s. | n.s. | n.s. |
| ABHD8 | −2.2 | n.s. | n.s. | n.s. | n.s. |
| ABI2 | −2.2 | n.s. | n.s. | n.s. | n.s. |
| ABL2 | 2.9 | n.s. | n.s. | n.s. | n.s. |
| ABTB2 | 2.6 | n.s. | n.s. | n.s. | n.s. |
| ACACA | −2.4 | n.s. | n.s. | n.s. | n.s. |
| ACAD10 | n.s. | n.s. | −2.1 | n.s. | n.s. |
| ACAD11 | −2.5 | n.s. | n.s. | n.s. | n.s. |
| ACADS | n.s. | n.s. | −2.0 | n.s. | n.s. |
| ACADSB | −2.2 | −2.1 | n.s. | n.s. | n.s. |
| ACBD4 | −2.9 | −2.3 | n.s. | n.s. | n.s. |
| ACE | −2.9 | n.s. | −2.7 | n.s. | n.s. |
| ACLY | n.s. | 2.2 | n.s. | n.s. | n.s. |
| ACOT11 | n.s. | 3.5 | n.s. | n.s. | n.s. |
| ACOT7 | 3.0 | n.s. | n.s. | n.s. | n.s. |
| ACOT8 | n.s. | −2.0 | n.s. | n.s. | n.s. |
| ACOX1 | −2.3 | n.s. | n.s. | n.s. | n.s. |
| ACOX2 | −2.0 | n.s. | n.s. | n.s. | n.s. |
| ACOX3 | n.s. | 1.9 | n.s. | n.s. | n.s. |
| ACP2 | n.s. | n.s. | −2.0 | n.s. | n.s. |
| ACRC | n.s. | −2.5 | n.s. | n.s. | n.s. |
| ACSBG1 | −3.3 | n.s. | n.s. | n.s. | n.s. |
| ACSL3 | −2.1 | n.s. | n.s. | n.s. | n.s. |
| ACSS3 | −2.0 | n.s. | n.s. | n.s. | n.s. |
| ACTA2 | n.s. | 2.0 | n.s. | n.s. | n.s. |
| ACTC1 | 5.1 | 2.6 | n.s. | n.s. | n.s. |
| ACTN1 | 1.9 | 2.5 | n.s. | n.s. | n.s. |
| ACTN4 | n.s. | 4.2 | n.s. | n.s. | n.s. |
| ACTR2 | n.s. | n.s. | n.s. | n.s. | −2.0 |
| ACTR3B | 2.7 | n.s. | n.s. | n.s. | n.s. |
| ACVR2A | −2.3 | −1.8 | n.s. | n.s. | n.s. |
| ACVRL1 | −2.9 | n.s. | n.s. | n.s. | n.s. |
| ADA | 2.2 | n.s. | n.s. | n.s. | n.s. |
| ADAL | −2.1 | n.s. | n.s. | n.s. | n.s. |
| ADAM10 | −2.1 | n.s. | n.s. | n.s. | n.s. |
| ADAM19 | 2.7 | 1.9 | n.s. | n.s. | n.s. |
| ADAM32 | n.s. | n.s. | n.s. | 3.9 | n.s. |
| ADAM33 | −3.5 | n.s. | n.s. | n.s. | n.s. |
| ADAM9 | −1.9 | n.s. | n.s. | n.s. | n.s. |
| ADAMTS1 | n.s. | 1.9 | n.s. | n.s. | n.s. |
| ADAMTS2 | n.s. | 2.0 | n.s. | n.s. | n.s. |
| ADAMTS5 | −2.0 | −1.9 | n.s. | n.s. | n.s. |
| ADAMTS6 | n.s. | 5.8 | n.s. | n.s. | n.s. |
| ADAMTSL1 | −2.5 | n.s. | n.s. | n.s. | n.s. |
| ADARB1 | −1.9 | n.s. | n.s. | n.s. | n.s. |
| ADAT1 | n.s. | n.s. | −1.8 | n.s. | n.s. |
| ADAT3 | 2.7 | n.s. | n.s. | n.s. | n.s. |
| ADCY3 | n.s. | −2.0 | n.s. | n.s. | n.s. |
| ADD1 | −1.9 | n.s. | n.s. | n.s. | n.s. |
| ADD3 | −3.1 | −1.9 | n.s. | n.s. | n.s. |
| ADFP | 1.9 | n.s. | n.s. | n.s. | n.s. |
| ADH1B | −7.1 | n.s. | n.s. | n.s. | n.s. |
| ADH5 | −1.9 | n.s. | n.s. | n.s. | n.s. |
| ADHFE1 | −3.1 | n.s. | n.s. | n.s. | n.s. |
| ADK | 2.1 | n.s. | n.s. | n.s. | n.s. |
| ADM | −1.9 | n.s. | n.s. | n.s. | n.s. |
| ADORA1 | n.s. | n.s. | −1.9 | n.s. | n.s. |
| ADORA2B | −3.1 | −2.2 | n.s. | n.s. | n.s. |
| ADORA3 | n.s. | n.s. | n.s. | −2.8 | n.s. |
| ADRA2A | −2.1 | n.s. | n.s. | n.s. | n.s. |
| ADRA2B | n.s. | n.s. | −6.6 | n.s. | n.s. |
| ADRA2C | 2.8 | 2.2 | n.s. | n.s. | n.s. |
| ADRM1 | 2.4 | n.s. | n.s. | n.s. | n.s. |
| AF034187 | n.s. | n.s. | 4.4 | n.s. | n.s. |
| AF086017 | n.s. | n.s. | n.s. | 1.8 | n.s. |
| AF086125 | n.s. | n.s. | n.s. | 2.6 | 2.0 |
| AF086187 | n.s. | n.s. | 1.9 | n.s. | n.s. |
| AF086205 | n.s. | n.s. | n.s. | n.s. | 2.3 |
| AF086329 | n.s. | n.s. | 1.9 | 2.0 | 2.2 |
| AF146694 | n.s. | n.s. | n.s. | 3.7 | n.s. |
| AF212044 | n.s. | n.s. | n.s. | n.s. | −1.8 |
| AF334588 | n.s. | n.s. | 5.0 | 4.0 | n.s. |
| AFF3 | −24.5 | −6.4 | n.s. | n.s. | n.s. |
| AFG3L2 | −2.0 | 1.9 | n.s. | n.s. | n.s. |
| AFP | 2.7 | n.s. | n.s. | n.s. | n.s. |
| AFTPH | n.s. | −1.9 | n.s. | n.s. | n.s. |
| AGBL5 | −2.2 | −2.3 | n.s. | n.s. | n.s. |
| AGL | −2.0 | n.s. | n.s. | n.s. | n.s. |
| AGPAT3 | −2.0 | n.s. | n.s. | n.s. | n.s. |
| AGPAT5 | 2.4 | n.s. | n.s. | n.s. | n.s. |
| AGPAT6 | 2.0 | n.s. | −2.0 | n.s. | n.s. |
| AGRN | −2.2 | n.s. | n.s. | n.s. | n.s. |
| AGXT | 2.7 | n.s. | n.s. | n.s. | n.s. |
| AGXT2L2 | −2.0 | n.s. | n.s. | n.s. | n.s. |
| AHCTF1 | n.s. | n.s. | n.s. | −2.1 | −2.8 |
| AHNAK | −2.1 | n.s. | n.s. | n.s. | n.s. |
| AHNAK2 | −2.5 | n.s. | n.s. | n.s. | n.s. |
| AHR | n.s. | −1.9 | n.s. | n.s. | n.s. |
| AHRR | −1.9 | −2.0 | n.s. | n.s. | n.s. |
| AHSA2 | −2.3 | n.s. | n.s. | n.s. | n.s. |
| AI051172 | n.s. | n.s. | n.s. | n.s. | 2.1 |
| AI161396 | n.s. | n.s. | n.s. | n.s. | 2.4 |
| AI192327 | n.s. | n.s. | 1.9 | n.s. | 2.0 |
| AI263083 | n.s. | n.s. | 2.0 | 2.0 | 1.8 |
| AI446524 | n.s. | n.s. | n.s. | n.s. | 30.6 |
| AI457687 | n.s. | n.s. | n.s. | 1.9 | n.s. |
| AI559980 | n.s. | n.s. | n.s. | 2.3 | n.s. |
| AI652920 | n.s. | n.s. | 2.0 | n.s. | 2.8 |
| AI709405 | n.s. | n.s. | n.s. | n.s. | 2.5 |
| AI873070 | n.s. | n.s. | n.s. | n.s. | 2.3 |
| AI925475 | n.s. | n.s. | n.s. | 1.8 | 1.8 |
| AIFM2 | 1.9 | n.s. | n.s. | n.s. | n.s. |
| AIM1 | −3.2 | −2.5 | n.s. | n.s. | n.s. |
| AJ295984 | n.s. | n.s. | n.s. | −2.1 | n.s. |
| AK000053 | n.s. | n.s. | n.s. | n.s. | −2.0 |
| AK001164 | n.s. | n.s. | n.s. | n.s. | 1.8 |
| AK021467 | n.s. | n.s. | 2.5 | n.s. | 3.9 |
| AK021606 | n.s. | n.s. | n.s. | 1.8 | n.s. |
| AK022044 | n.s. | n.s. | 1.8 | n.s. | n.s. |
| AK022268 | n.s. | n.s. | 2.6 | n.s. | n.s. |
| AK022339 | n.s. | n.s. | 1.9 | n.s. | n.s. |
| AK022479 | n.s. | n.s. | 3.0 | n.s. | n.s. |
| AK023328 | n.s. | n.s. | 2.3 | n.s. | n.s. |
| AK023647 | n.s. | n.s. | n.s. | n.s. | 1.9 |
| AK026418 | n.s. | n.s. | 1.8 | n.s. | n.s. |
| AK026984 | n.s. | n.s. | n.s. | n.s. | 2.2 |
| AK055302 | n.s. | n.s. | 2.0 | 1.9 | n.s. |
| AK074181 | n.s. | n.s. | 2.1 | 2.1 | 1.9 |
| AK075186 | n.s. | n.s. | 2.1 | 1.8 | n.s. |
| AK092379 | n.s. | n.s. | 2.0 | n.s. | n.s. |
| AK092668 | n.s. | n.s. | 1.8 | 1.8 | n.s. |
| AK093508 | n.s. | n.s. | 5.5 | 5.7 | n.s. |
| AK094296 | n.s. | n.s. | n.s. | n.s. | 2.1 |
| AK094571 | n.s. | n.s. | n.s. | n.s. | 2.2 |
| AK124806 | n.s. | n.s. | n.s. | 2.3 | n.s. |
| AK126245 | n.s. | n.s. | n.s. | 2.1 | n.s. |
| AK131472 | n.s. | n.s. | n.s. | 1.8 | n.s. |
| AKAP1 | n.s. | 2.0 | n.s. | n.s. | n.s. |
| AKAP11 | −2.2 | n.s. | n.s. | n.s. | n.s. |
| AKAP12 | −2.4 | 3.1 | n.s. | −2.0 | n.s. |
| AKAP8 | 2.0 | n.s. | n.s. | n.s. | n.s. |
| AKNA | −3.4 | −2.2 | n.s. | n.s. | n.s. |
| AKR1B10 | 5.2 | n.s. | n.s. | n.s. | n.s. |
| AKR1C1 | 9.9 | −2.4 | n.s. | n.s. | n.s. |
| AKR1C2 | 7.6 | −1.9 | n.s. | n.s. | n.s. |
| AKR1C3 | n.s. | −4.2 | n.s. | n.s. | n.s. |
| AKR1C4 | 3.3 | −1.9 | n.s. | n.s. | n.s. |
| AKR1CL2 | 2.2 | n.s. | n.s. | n.s. | n.s. |
| AKT1 | n.s. | 1.8 | −1.8 | n.s. | n.s. |
| AKT2 | −3.1 | n.s. | n.s. | n.s. | n.s. |
| AKT3 | −2.4 | n.s. | n.s. | n.s. | n.s. |
| AKTIP | n.s. | −2.4 | n.s. | n.s. | n.s. |
| AL040873 | n.s. | n.s. | n.s. | 2.5 | n.s. |
| AL041007 | n.s. | n.s. | 1.8 | n.s. | n.s. |
| AL547361 | n.s. | n.s. | n.s. | n.s. | 2.6 |
| AL566369 | n.s. | n.s. | n.s. | 1.9 | n.s. |
| AL571926 | n.s. | n.s. | n.s. | 1.9 | 1.8 |
| AL833114 | n.s. | n.s. | 2.0 | n.s. | n.s. |
| ALAS1 | 1.9 | n.s. | n.s. | n.s. | n.s. |
| ALCAM | n.s. | 2.5 | n.s. | n.s. | n.s. |
| ALDH1B1 | n.s. | 1.8 | n.s. | n.s. | n.s. |
| ALDH1L2 | −2.9 | n.s. | n.s. | n.s. | n.s. |
| ALDH3A1 | −5.1 | −7.9 | n.s. | −2.2 | n.s. |
| ALDH3A2 | −2.4 | −2.1 | n.s. | n.s. | n.s. |
| ALDH3B1 | −1.8 | n.s. | n.s. | n.s. | n.s. |
| ALDH6A1 | −3.1 | −1.8 | n.s. | n.s. | n.s. |
| ALDH7A1 | −2.4 | n.s. | n.s. | n.s. | n.s. |
| ALG10B | −2.5 | −1.9 | n.s. | n.s. | n.s. |
| ALG11 | n.s. | −2.1 | n.s. | n.s. | n.s. |
| ALG3 | 2.1 | n.s. | n.s. | n.s. | n.s. |
| ALMS1 | −2.0 | n.s. | n.s. | n.s. | n.s. |
| ALOX12 | n.s. | −3.9 | n.s. | n.s. | n.s. |
| ALPK1 | −3.2 | −1.9 | n.s. | n.s. | n.s. |
| ALPK2 | n.s. | 2.0 | n.s. | n.s. | n.s. |
| ALS2CR3 | 2.2 | n.s. | n.s. | n.s. | n.s. |
| ALS2CR7 | n.s. | −1.9 | n.s. | n.s. | n.s. |
| ALS2CR8 | −2.4 | n.s. | n.s. | n.s. | n.s. |
| ALX3 | −2.0 | n.s. | n.s. | n.s. | n.s. |
| AMAC1L2 | n.s. | n.s. | n.s. | n.s. | −2.3 |
| AMDHD2 | 2.7 | n.s. | n.s. | n.s. | n.s. |
| AMIGO2 | 2.6 | 6.1 | n.s. | n.s. | n.s. |
| AMOT | −2.4 | n.s. | n.s. | n.s. | n.s. |
| AMOTL1 | −2.0 | n.s. | n.s. | n.s. | n.s. |
| AMPH | −2.8 | n.s. | n.s. | n.s. | n.s. |
| AMT | −3.3 | n.s. | n.s. | n.s. | n.s. |
| AMZ2 | −2.0 | n.s. | n.s. | n.s. | n.s. |
| ANAPC5 | −2.1 | n.s. | n.s. | n.s. | n.s. |
| ANAPC7 | n.s. | n.s. | −1.8 | −1.8 | n.s. |
| ANG | −2.3 | −2.0 | n.s. | n.s. | n.s. |
| ANGEL2 | −2.0 | n.s. | n.s. | n.s. | n.s. |
| ANGPTL2 | −2.4 | −1.9 | n.s. | n.s. | n.s. |
| ANGPTL4 | 4.0 | 5.2 | n.s. | −2.1 | n.s. |
| ANK1 | −13.3 | n.s. | 2.0 | n.s. | n.s. |
| ANKDD1A | −3.1 | −2.1 | n.s. | n.s. | n.s. |
| ANKFN1 | n.s. | −4.1 | n.s. | n.s. | n.s. |
| ANKH | 1.9 | 3.7 | n.s. | n.s. | n.s. |
| ANKIB1 | −1.9 | −1.8 | n.s. | n.s. | n.s. |
| ANKRA2 | n.s. | −1.8 | n.s. | n.s. | n.s. |
| ANKRD10 | −2.9 | −4.5 | n.s. | n.s. | n.s. |
| ANKRD11 | n.s. | 2.6 | n.s. | n.s. | n.s. |
| ANKRD12 | −1.9 | −1.8 | n.s. | n.s. | n.s. |
| ANKRD13C | 2.3 | n.s. | n.s. | n.s. | n.s. |
| ANKRD13D | n.s. | n.s. | n.s. | −1.9 | n.s. |
| ANKRD17 | 2.1 | 2.1 | n.s. | n.s. | n.s. |
| ANKRD26 | −2.8 | n.s. | n.s. | n.s. | n.s. |
| ANKRD28 | −4.7 | 2.0 | n.s. | n.s. | n.s. |
| ANKRD29 | −2.4 | n.s. | n.s. | n.s. | n.s. |
| ANKRD37 | 2.3 | n.s. | n.s. | n.s. | n.s. |
| ANKRD38 | 9.0 | n.s. | n.s. | n.s. | n.s. |
| ANKRD42 | −2.1 | −2.3 | n.s. | n.s. | n.s. |
| ANKRD44 | n.s. | n.s. | −7.4 | n.s. | n.s. |
| ANKRD50 | −1.8 | −1.8 | n.s. | n.s. | n.s. |
| ANKRD57 | 2.5 | 2.0 | n.s. | n.s. | n.s. |
| ANKRD9 | 3.5 | n.s. | n.s. | n.s. | n.s. |
| ANLN | 2.5 | n.s. | n.s. | n.s. | n.s. |
| ANP32D | n.s. | n.s. | −1.8 | n.s. | n.s. |
| ANP32E | 2.1 | −2.1 | n.s. | n.s. | n.s. |
| ANTXR2 | n.s. | 2.0 | n.s. | n.s. | n.s. |
| ANXA11 | −9.9 | n.s. | n.s. | n.s. | n.s. |
| ANXA2P1 | n.s. | n.s. | −1.9 | n.s. | n.s. |
| ANXA3 | −1.9 | n.s. | n.s. | n.s. | n.s. |
| ANXA4 | −1.9 | n.s. | n.s. | n.s. | n.s. |
| ANXA8L2 | 2.6 | n.s. | n.s. | n.s. | n.s. |
| AOC2 | 4.4 | n.s. | n.s. | n.s. | n.s. |
| AOF2 | −1.9 | −3.0 | n.s. | n.s. | n.s. |
| AOX1 | −1.9 | n.s. | n.s. | n.s. | n.s. |
| AP1GBP1 | n.s. | −2.6 | n.s. | n.s. | n.s. |
| AP1S1 | n.s. | n.s. | −1.9 | n.s. | n.s. |
| AP2B1 | −1.9 | n.s. | n.s. | n.s. | n.s. |
| AP4S1 | −2.1 | n.s. | n.s. | n.s. | n.s. |
| APBB2 | n.s. | 2.5 | n.s. | n.s. | n.s. |
| APCDD1 | −36.6 | −9.2 | n.s. | n.s. | n.s. |
| APCDD1L | 2.2 | n.s. | n.s. | n.s. | n.s. |
| API5 | 1.9 | n.s. | n.s. | n.s. | n.s. |
| APOBEC3B | 2.4 | n.s. | n.s. | n.s. | n.s. |
| APOBEC3C | −1.9 | n.s. | −1.8 | n.s. | n.s. |
| APOBEC3F | n.s. | −2.0 | n.s. | n.s. | n.s. |
| APOBEC3G | n.s. | n.s. | n.s. | n.s. | 5.0 |
| APOD | −2.8 | n.s. | n.s. | n.s. | n.s. |
| APOL2 | n.s. | n.s. | −2.0 | n.s. | n.s. |
| APOL3 | −11.1 | n.s. | n.s. | n.s. | n.s. |
| APOL6 | −1.9 | −1.8 | n.s. | n.s. | n.s. |
| APOLD1 | n.s. | −2.9 | n.s. | n.s. | n.s. |
| APOOL | −1.8 | −2.3 | n.s. | n.s. | n.s. |
| APP | −1.8 | n.s. | n.s. | n.s. | n.s. |
| APPBP2 | −2.1 | n.s. | n.s. | n.s. | n.s. |
| APPL1 | −1.9 | −2.0 | n.s. | n.s. | n.s. |
| AQP1 | −3.5 | n.s. | n.s. | n.s. | n.s. |
| AQP10 | n.s. | n.s. | n.s. | n.s. | 4.0 |
| ARC | 5.8 | 4.1 | n.s. | n.s. | n.s. |
| ARD1A | 2.1 | n.s. | n.s. | n.s. | n.s. |
| ARF1 | n.s. | 1.8 | n.s. | n.s. | n.s. |
| ARF3 | n.s. | n.s. | −1.8 | n.s. | n.s. |
| ARF5 | −2.4 | −1.8 | n.s. | n.s. | n.s. |
| ARFGAP1 | n.s. | n.s. | −2.3 | n.s. | −2.2 |
| ARFRP1 | n.s. | n.s. | −1.9 | n.s. | n.s. |
| ARG2 | 2.9 | n.s. | n.s. | n.s. | n.s. |
| ARHGAP12 | −2.8 | n.s. | n.s. | n.s. | n.s. |
| ARHGAP18 | −1.9 | n.s. | n.s. | n.s. | n.s. |
| ARHGAP20 | n.s. | n.s. | 2.0 | n.s. | n.s. |
| ARHGAP22 | 2.5 | 2.8 | n.s. | n.s. | n.s. |
| ARHGAP23 | −2.0 | n.s. | n.s. | n.s. | n.s. |
| ARHGAP26 | −6.7 | n.s. | n.s. | n.s. | n.s. |
| ARHGAP27 | n.s. | n.s. | n.s. | n.s. | 57.0 |
| ARHGAP28 | −30.6 | −5.5 | n.s. | n.s. | n.s. |
| ARHGAP29 | −3.2 | −1.9 | n.s. | n.s. | n.s. |
| ARHGAP5 | −1.8 | n.s. | n.s. | n.s. | n.s. |
| ARHGDIA | 2.3 | n.s. | n.s. | n.s. | n.s. |
| ARHGEF10L | −2.9 | n.s. | n.s. | n.s. | n.s. |
| ARHGEF12 | −1.9 | n.s. | n.s. | n.s. | n.s. |
| ARHGEF17 | −2.7 | n.s. | n.s. | n.s. | n.s. |
| ARHGEF3 | −2.0 | n.s. | n.s. | n.s. | n.s. |
| ARHGEF6 | −2.3 | n.s. | n.s. | n.s. | n.s. |
| ARHGEF7 | −5.5 | −9.0 | n.s. | n.s. | n.s. |
| ARID4A | −2.1 | −2.4 | n.s. | n.s. | n.s. |
| ARID5B | −2.0 | n.s. | n.s. | n.s. | n.s. |
| ARIH1 | 2.1 | n.s. | n.s. | n.s. | n.s. |
| ARL17 | 1.9 | n.s. | n.s. | n.s. | n.s. |
| ARL5A | −3.0 | n.s. | n.s. | n.s. | n.s. |
| ARL6IP1 | n.s. | −1.9 | n.s. | n.s. | n.s. |
| ARL8B | 2.2 | n.s. | n.s. | n.s. | n.s. |
| ARMC7 | 2.0 | n.s. | n.s. | n.s. | n.s. |
| ARMC9 | −2.6 | −2.1 | n.s. | n.s. | n.s. |
| ARMCX2 | −1.9 | n.s. | n.s. | n.s. | n.s. |
| ARMCX3 | −2.5 | n.s. | n.s. | n.s. | n.s. |
| ARMCX4 | −4.2 | n.s. | n.s. | n.s. | n.s. |
| ARMET | 3.3 | n.s. | n.s. | n.s. | n.s. |
| ARNT | −2.0 | n.s. | n.s. | n.s. | n.s. |
| ARNT2 | −4.9 | n.s. | n.s. | n.s. | n.s. |
| ARNTL | 2.6 | n.s. | n.s. | n.s. | n.s. |
| ARNTL2 | 3.0 | n.s. | n.s. | n.s. | n.s. |
| ARPC4 | n.s. | 2.1 | n.s. | n.s. | n.s. |
| ARPC5L | 2.3 | n.s. | n.s. | n.s. | n.s. |
| ARPP-19 | 2.0 | n.s. | n.s. | n.s. | n.s. |
| ARRDC4 | 2.1 | n.s. | n.s. | n.s. | n.s. |
| ARSD | −2.4 | n.s. | n.s. | n.s. | n.s. |
| ARSI | 5.3 | 1.9 | n.s. | n.s. | n.s. |
| ARTS-1 | −2.1 | n.s. | n.s. | n.s. | n.s. |
| ARVCF | n.s. | n.s. | n.s. | −1.8 | n.s. |
| ASAH2B | 3.1 | 2.5 | n.s. | n.s. | n.s. |
| ASB6 | 2.7 | n.s. | n.s. | n.s. | n.s. |
| ASF1B | 4.4 | n.s. | n.s. | n.s. | n.s. |
| ASPA | −4.0 | n.s. | n.s. | n.s. | n.s. |
| ASPH | 2.3 | n.s. | n.s. | n.s. | n.s. |
| ASPHD1 | 7.1 | n.s. | n.s. | n.s. | n.s. |
| ASPM | 4.3 | −2.1 | n.s. | n.s. | n.s. |
| ASPN | −3.1 | n.s. | n.s. | n.s. | n.s. |
| ASS1 | −2.2 | n.s. | n.s. | n.s. | n.s. |
| ASXL1 | 2.1 | n.s. | n.s. | n.s. | n.s. |
| ATAD2 | 2.1 | n.s. | n.s. | n.s. | n.s. |
| ATAD2B | −2.5 | −2.6 | n.s. | n.s. | n.s. |
| ATAD3A | 4.0 | 2.4 | n.s. | n.s. | n.s. |
| ATAD3B | 4.9 | 3.9 | −1.9 | n.s. | n.s. |
| ATF3 | 42.8 | 16.2 | n.s. | n.s. | n.s. |
| ATF6 | −2.0 | n.s. | n.s. | n.s. | n.s. |
| ATF7IP | n.s. | n.s. | n.s. | n.s. | −2.4 |
| ATM | −2.8 | n.s. | n.s. | n.s. | n.s. |
| ATOH8 | 5.0 | 2.2 | n.s. | n.s. | n.s. |
| ATP10A | n.s. | 2.4 | n.s. | n.s. | n.s. |
| ATP11B | −2.1 | n.s. | n.s. | n.s. | n.s. |
| ATP1A2 | n.s. | n.s. | 2.1 | 2.0 | n.s. |
| ATP1B3 | 4.0 | n.s. | n.s. | n.s. | n.s. |
| ATP2A1 | 7.1 | n.s. | n.s. | n.s. | n.s. |
| ATP2B1 | −2.0 | 2.2 | n.s. | n.s. | n.s. |
| ATP2B3 | n.s. | n.s. | n.s. | −2.1 | n.s. |
| ATP2C1 | −2.5 | n.s. | n.s. | n.s. | n.s. |
| ATP5C1 | −2.3 | −1.8 | n.s. | n.s. | n.s. |
| ATP5S | −1.9 | n.s. | n.s. | n.s. | n.s. |
| ATP6V0B | 3.0 | n.s. | n.s. | n.s. | n.s. |
| ATP6V0D2 | 4.9 | n.s. | n.s. | n.s. | n.s. |
| ATP6V0E | 1.8 | n.s. | n.s. | n.s. | n.s. |
| ATP6V0E1 | 2.7 | n.s. | n.s. | n.s. | n.s. |
| ATP6V0E2 | −1.8 | n.s. | −1.8 | n.s. | n.s. |
| ATP6V1C1 | 2.1 | n.s. | n.s. | n.s. | n.s. |
| ATP6V1C2 | 12.9 | n.s. | n.s. | n.s. | n.s. |
| ATP6V1G2 | −4.2 | n.s. | n.s. | n.s. | n.s. |
| ATP6V1H | 1.9 | n.s. | n.s. | n.s. | n.s. |
| ATP7A | −2.0 | n.s. | n.s. | n.s. | n.s. |
| ATP8A2 | n.s. | n.s. | n.s. | n.s. | 1.8 |
| ATP8B1 | −4.8 | 1.8 | n.s. | n.s. | n.s. |
| ATPBD1C | 3.2 | n.s. | n.s. | n.s. | n.s. |
| ATRIP | 2.2 | n.s. | n.s. | n.s. | n.s. |
| ATRX | −2.3 | 2.0 | n.s. | n.s. | n.s. |
| ATXN1 | 1.9 | −2.2 | n.s. | n.s. | n.s. |
| ATXN1L | n.s. | −1.9 | n.s. | n.s. | n.s. |
| ATXN3 | −2.0 | n.s. | n.s. | n.s. | n.s. |
| ATXN7L1 | −2.3 | n.s. | n.s. | n.s. | n.s. |
| AUP1 | 1.9 | n.s. | n.s. | n.s. | n.s. |
| AURKA | 3.0 | n.s. | n.s. | n.s. | n.s. |
| AURKB | 6.1 | 2.8 | −2.0 | n.s. | n.s. |
| AV702101 | n.s. | n.s. | 1.9 | n.s. | n.s. |
| AW167080 | n.s. | n.s. | n.s. | 2.0 | n.s. |
| AW191706 | n.s. | n.s. | n.s. | 1.9 | n.s. |
| AW858928 | n.s. | n.s. | n.s. | 1.9 | n.s. |
| AW885990 | n.s. | n.s. | n.s. | 2.4 | n.s. |
| AW901755 | n.s. | n.s. | n.s. | n.s. | 5.3 |
| AXIN2 | −3.6 | −2.4 | n.s. | n.s. | n.s. |
| AXUD1 | 4.6 | 4.1 | n.s. | n.s. | n.s. |
| AZI2 | −1.9 | −1.9 | n.s. | n.s. | n.s. |
| AZIN1 | n.s. | −3.8 | n.s. | n.s. | n.s. |
| B3GALNT1 | −1.8 | n.s. | n.s. | n.s. | n.s. |
| B3GALT2 | −11.8 | n.s. | n.s. | n.s. | n.s. |
| B3GALT4 | −2.4 | −3.0 | n.s. | n.s. | n.s. |
| B3GALT6 | 3.7 | 1.8 | n.s. | n.s. | n.s. |
| B3GAT3 | 2.1 | n.s. | n.s. | n.s. | n.s. |
| B4GALNT1 | 1.8 | n.s. | n.s. | n.s. | n.s. |
| B4GALT1 | 2.1 | 1.9 | n.s. | n.s. | n.s. |
| B9D1 | −2.5 | n.s. | n.s. | n.s. | n.s. |
| BAALC | 2.1 | n.s. | n.s. | n.s. | n.s. |
| BACE1 | −7.5 | −4.9 | n.s. | n.s. | n.s. |
| BAG1 | 2.4 | n.s. | n.s. | n.s. | n.s. |
| BAG2 | −2.3 | n.s. | n.s. | n.s. | n.s. |
| BAG4 | n.s. | −1.9 | n.s. | n.s. | n.s. |
| BAIAP2 | 2.0 | −2.6 | n.s. | n.s. | n.s. |
| BAIAP2L1 | n.s. | 2.0 | n.s. | n.s. | n.s. |
| BAK1 | 2.3 | n.s. | n.s. | n.s. | n.s. |
| BAMBI | 6.5 | 2.2 | n.s. | n.s. | n.s. |
| BANK1 | n.s. | n.s. | n.s. | n.s. | 2.7 |
| BAP1 | n.s. | n.s. | −1.9 | n.s. | n.s. |
| BAT1 | n.s. | n.s. | −1.9 | n.s. | n.s. |
| BAT2D1 | n.s. | 2.5 | n.s. | n.s. | n.s. |
| BAX | n.s. | n.s. | −1.8 | n.s. | n.s. |
| BBS1 | −2.1 | n.s. | n.s. | n.s. | n.s. |
| BBS2 | −2.2 | n.s. | n.s. | n.s. | n.s. |
| BBS9 | −2.5 | n.s. | n.s. | n.s. | n.s. |
| BC008476 | n.s. | n.s. | 2.6 | n.s. | 1.9 |
| BC015334 | n.s. | n.s. | n.s. | 2.2 | 4.0 |
| BC015449 | n.s. | n.s. | 1.9 | 2.2 | n.s. |
| BC019907 | n.s. | n.s. | n.s. | 1.9 | n.s. |
| BC032901 | n.s. | n.s. | n.s. | −5.4 | n.s. |
| BC036599 | n.s. | n.s. | 1.8 | 1.9 | n.s. |
| BC036928 | 1.9 | n.s. | n.s. | n.s. | n.s. |
| BC047110 | n.s. | n.s. | 2.1 | 1.9 | n.s. |
| BCAP31 | n.s. | n.s. | −1.9 | n.s. | n.s. |
| BCAR3 | n.s. | 2.6 | n.s. | n.s. | n.s. |
| BCAR4 | 3.9 | n.s. | n.s. | n.s. | n.s. |
| BCAS3 | −2.2 | n.s. | n.s. | n.s. | n.s. |
| BCHE | n.s. | n.s. | 1.9 | 2.1 | 2.1 |
| BCKDHB | −2.7 | −2.1 | n.s. | n.s. | n.s. |
| BCL11A | −5.0 | −3.3 | n.s. | n.s. | n.s. |
| BCL2L11 | 2.9 | n.s. | n.s. | n.s. | n.s. |
| BCL2L12 | 2.6 | n.s. | n.s. | n.s. | n.s. |
| BCL6 | 2.2 | 4.7 | n.s. | n.s. | n.s. |
| BCL7A | 2.3 | n.s. | n.s. | n.s. | n.s. |
| BCLAF1 | 2.1 | n.s. | n.s. | n.s. | n.s. |
| BCR | −2.2 | n.s. | n.s. | n.s. | n.s. |
| BDH2 | −2.8 | n.s. | n.s. | n.s. | n.s. |
| BDKRB1 | 3.0 | n.s. | n.s. | n.s. | n.s. |
| BDKRB2 | −2.7 | n.s. | n.s. | n.s. | n.s. |
| BDNF | 2.1 | 1.9 | n.s. | n.s. | n.s. |
| BE379389 | n.s. | n.s. | −2.0 | n.s. | n.s. |
| BE644757 | n.s. | n.s. | n.s. | 2.0 | n.s. |
| BE719776 | n.s. | n.s. | 2.1 | 2.1 | 2.4 |
| BE766438 | n.s. | n.s. | n.s. | 3.8 | n.s. |
| BEX1 | 3.9 | n.s. | n.s. | n.s. | n.s. |
| BEXL1 | −2.4 | n.s. | n.s. | n.s. | n.s. |
| BF195626 | n.s. | n.s. | n.s. | 1.8 | n.s. |
| BHLHB2 | 8.8 | 7.7 | n.s. | n.s. | n.s. |
| BHLHB3 | 2.3 | n.s. | n.s. | n.s. | n.s. |
| BI836739 | n.s. | n.s. | 3.9 | n.s. | n.s. |
| BICC1 | −2.5 | n.s. | n.s. | n.s. | n.s. |
| BICD1 | 5.3 | 1.8 | n.s. | n.s. | n.s. |
| BIRC4 | 1.9 | −2.0 | n.s. | n.s. | n.s. |
| BIRC5 | 4.8 | −2.1 | n.s. | n.s. | n.s. |
| BIVM | −2.6 | n.s. | n.s. | n.s. | n.s. |
| BLCAP | −1.9 | n.s. | n.s. | n.s. | n.s. |
| BLM | 3.3 | n.s. | n.s. | n.s. | n.s. |
| BLOC1S1 | −2.1 | n.s. | n.s. | n.s. | n.s. |
| BLOC1S3 | n.s. | −2.8 | n.s. | n.s. | n.s. |
| BM023 | 3.9 | n.s. | n.s. | n.s. | n.s. |
| BM455859 | n.s. | n.s. | n.s. | −1.8 | −2.0 |
| BM986990 | n.s. | n.s. | 2.0 | n.s. | n.s. |
| BMP2 | 11.1 | 8.4 | n.s. | n.s. | n.s. |
| BMP2K | −2.0 | n.s. | n.s. | n.s. | n.s. |
| BMP6 | n.s. | 2.4 | n.s. | n.s. | n.s. |
| BMPER | −2.7 | 1.9 | n.s. | n.s. | n.s. |
| BMPR1B | 3.3 | n.s. | n.s. | n.s. | n.s. |
| BMPR2 | −2.1 | 3.3 | n.s. | n.s. | n.s. |
| BNC1 | n.s. | 2.0 | n.s. | n.s. | n.s. |
| BNC2 | −3.3 | −2.5 | n.s. | n.s. | n.s. |
| BNIP2 | −2.6 | −2.1 | n.s. | n.s. | n.s. |
| BOC | 2.4 | n.s. | n.s. | n.s. | n.s. |
| BOK | n.s. | 2.3 | n.s. | n.s. | n.s. |
| BOLA2 | 2.4 | n.s. | n.s. | n.s. | n.s. |
| BOP1 | 2.7 | 2.4 | n.s. | n.s. | n.s. |
| BP872463 | n.s. | n.s. | n.s. | n.s. | 1.9 |
| BPGM | 2.3 | n.s. | n.s. | n.s. | n.s. |
| BPI | n.s. | n.s. | n.s. | n.s. | 7.7 |
| BPTF | −2.1 | −2.1 | n.s. | n.s. | n.s. |
| BQ000605 | n.s. | n.s. | 2.3 | 2.7 | n.s. |
| BQ772270 | n.s. | n.s. | n.s. | n.s. | −2.0 |
| BRAP | 2.0 | n.s. | n.s. | n.s. | n.s. |
| BRD2 | 2.4 | n.s. | n.s. | n.s. | n.s. |
| BRD4 | −3.6 | n.s. | −2.0 | n.s. | n.s. |
| BRD8 | n.s. | −2.5 | n.s. | n.s. | n.s. |
| BRF2 | 2.2 | n.s. | n.s. | n.s. | n.s. |
| BRIP1 | 2.2 | n.s. | n.s. | n.s. | n.s. |
| BRMS1L | n.s. | −2.3 | n.s. | n.s. | n.s. |
| BRP44 | 2.4 | n.s. | n.s. | n.s. | n.s. |
| BRWD1 | −1.9 | −2.3 | n.s. | n.s. | n.s. |
| BTBD14A | 3.4 | n.s. | n.s. | n.s. | n.s. |
| BTBD2 | −2.8 | n.s. | n.s. | n.s. | n.s. |
| BTBD7 | n.s. | −2.0 | n.s. | n.s. | n.s. |
| BTD | −1.8 | n.s. | n.s. | n.s. | n.s. |
| BTG1 | 2.2 | 2.1 | n.s. | n.s. | n.s. |
| BTG3 | 1.9 | n.s. | n.s. | n.s. | n.s. |
| BTN3A1 | −2.1 | −2.4 | n.s. | n.s. | n.s. |
| BTN3A2 | −1.9 | −1.9 | n.s. | n.s. | n.s. |
| BTN3A3 | −2.1 | n.s. | n.s. | n.s. | n.s. |
| BU160948 | n.s. | n.s. | n.s. | 2.2 | n.s. |
| BU561469 | n.s. | n.s. | n.s. | −1.9 | n.s. |
| BUB1 | 3.5 | n.s. | n.s. | n.s. | n.s. |
| BUB1B | 4.0 | n.s. | n.s. | n.s. | n.s. |
| BVES | 2.6 | n.s. | n.s. | n.s. | n.s. |
| BX098411 | n.s. | n.s. | n.s. | 2.0 | n.s. |
| BX100298 | n.s. | n.s. | n.s. | −3.0 | n.s. |
| BX412469 | n.s. | n.s. | 2.2 | n.s. | n.s. |
| BX433326 | n.s. | n.s. | n.s. | 2.8 | n.s. |
| BX448200 | n.s. | n.s. | n.s. | n.s. | 1.9 |
| BXDC2 | 1.9 | n.s. | n.s. | n.s. | n.s. |
| BYSL | 2.7 | 2.1 | n.s. | n.s. | n.s. |
| C10orf10 | n.s. | n.s. | −2.3 | n.s. | n.s. |
| C10orf104 | −1.9 | n.s. | n.s. | n.s. | n.s. |
| C10orf107 | −5.9 | n.s. | n.s. | n.s. | n.s. |
| C10orf11 | −2.7 | n.s. | n.s. | n.s. | n.s. |
| C10orf118 | −1.9 | −2.4 | n.s. | n.s. | n.s. |
| C10orf119 | n.s. | 1.9 | n.s. | n.s. | n.s. |
| C10orf125 | 2.0 | n.s. | n.s. | n.s. | n.s. |
| C10orf140 | n.s. | −4.6 | n.s. | n.s. | n.s. |
| C10orf18 | 3.9 | n.s. | n.s. | n.s. | n.s. |
| C10orf27 | n.s. | n.s. | n.s. | n.s. | 1.9 |
| C10orf32 | −1.9 | −2.0 | n.s. | n.s. | n.s. |
| C10orf33 | −12.2 | −3.2 | n.s. | n.s. | n.s. |
| C10orf39 | n.s. | n.s. | n.s. | −2.1 | n.s. |
| C10orf46 | n.s. | −1.9 | n.s. | n.s. | n.s. |
| C10orf54 | −5.7 | −2.2 | n.s. | n.s. | n.s. |
| C10orf56 | n.s. | −2.7 | n.s. | n.s. | n.s. |
| C10orf57 | −1.9 | n.s. | n.s. | n.s. | n.s. |
| C10orf59 | n.s. | n.s. | n.s. | n.s. | 3.5 |
| C10orf72 | −2.0 | n.s. | n.s. | n.s. | n.s. |
| C10orf90 | 4.2 | 1.8 | n.s. | n.s. | n.s. |
| C10orf91 | n.s. | n.s. | 2.9 | n.s. | n.s. |
| C11orf17 | 2.4 | n.s. | n.s. | n.s. | n.s. |
| C11orf21 | n.s. | n.s. | n.s. | −3.2 | n.s. |
| C11orf30 | n.s. | −2.1 | n.s. | n.s. | n.s. |
| C11orf31 | n.s. | −2.2 | n.s. | n.s. | n.s. |
| C11orf32 | −3.0 | n.s. | n.s. | n.s. | n.s. |
| C11orf34 | −1.9 | −2.2 | n.s. | n.s. | n.s. |
| C11orf41 | n.s. | 2.9 | n.s. | n.s. | n.s. |
| C11orf46 | −2.3 | −1.9 | n.s. | n.s. | n.s. |
| C11orf48 | 1.9 | n.s. | n.s. | n.s. | n.s. |
| C11orf54 | −1.9 | n.s. | n.s. | n.s. | n.s. |
| C11orf57 | −1.9 | n.s. | n.s. | n.s. | n.s. |
| C11orf70 | −2.2 | −1.9 | n.s. | n.s. | n.s. |
| C11orf73 | n.s. | n.s. | 2.2 | n.s. | n.s. |
| C11orf82 | 5.8 | 1.8 | n.s. | n.s. | n.s. |
| C12orf30 | n.s. | 2.0 | n.s. | n.s. | n.s. |
| C12orf41 | n.s. | n.s. | −1.9 | −2.0 | n.s. |
| C12orf42 | n.s. | n.s. | 5.1 | 5.7 | 5.4 |
| C12orf48 | 1.9 | n.s. | n.s. | n.s. | n.s. |
| C12orf49 | 3.0 | n.s. | n.s. | n.s. | n.s. |
| C12orf56 | 3.2 | n.s. | n.s. | n.s. | n.s. |
| C13orf1 | n.s. | n.s. | n.s. | n.s. | 1.8 |
| C13orf15 | −3.3 | n.s. | n.s. | n.s. | n.s. |
| C13orf18 | n.s. | 3.3 | n.s. | n.s. | n.s. |
| C13orf3 | 2.8 | n.s. | n.s. | n.s. | n.s. |
| C13orf31 | n.s. | −2.3 | n.s. | n.s. | n.s. |
| C13orf33 | n.s. | 3.5 | n.s. | n.s. | n.s. |
| C14orf1 | −2.8 | −3.1 | n.s. | n.s. | n.s. |
| C14orf101 | −1.8 | n.s. | n.s. | n.s. | n.s. |
| C14orf115 | n.s. | n.s. | n.s. | −2.4 | n.s. |
| C14orf122 | 2.2 | n.s. | n.s. | n.s. | n.s. |
| C14orf132 | n.s. | −2.1 | n.s. | n.s. | n.s. |
| C14orf133 | −2.0 | n.s. | n.s. | n.s. | n.s. |
| C14orf138 | 2.3 | n.s. | n.s. | n.s. | n.s. |
| C14orf139 | −3.4 | −1.9 | n.s. | n.s. | n.s. |
| C14orf145 | 2.7 | n.s. | n.s. | n.s. | n.s. |
| C14orf159 | −1.9 | n.s. | n.s. | n.s. | n.s. |
| C14orf167 | −4.3 | −2.9 | n.s. | n.s. | n.s. |
| C14orf172 | 2.1 | n.s. | n.s. | n.s. | n.s. |
| C14orf173 | 2.2 | n.s. | n.s. | n.s. | n.s. |
| C14orf43 | −1.9 | n.s. | n.s. | n.s. | n.s. |
| C14orf49 | n.s. | n.s. | n.s. | n.s. | 1.9 |
| C14orf80 | 4.1 | n.s. | n.s. | n.s. | n.s. |
| C15orf23 | 2.2 | −1.9 | n.s. | n.s. | n.s. |
| C15orf33 | −2.7 | n.s. | n.s. | n.s. | n.s. |
| C15orf38 | −2.4 | n.s. | n.s. | n.s. | n.s. |
| C15orf40 | n.s. | −2.5 | n.s. | n.s. | n.s. |
| C15orf41 | −2.1 | n.s. | n.s. | n.s. | n.s. |
| C15orf48 | 8.8 | n.s. | n.s. | n.s. | n.s. |
| C15orf5 | n.s. | n.s. | 3.7 | n.s. | n.s. |
| C15orf52 | n.s. | −1.9 | n.s. | n.s. | n.s. |
| C16orf44 | −3.5 | −2.4 | n.s. | n.s. | n.s. |
| C16orf45 | −2.1 | n.s. | n.s. | n.s. | n.s. |
| C16orf53 | 2.6 | n.s. | n.s. | n.s. | n.s. |
| C16orf55 | 2.3 | n.s. | n.s. | n.s. | n.s. |
| C16orf57 | n.s. | 2.2 | n.s. | n.s. | n.s. |
| C16orf59 | 3.5 | 2.5 | n.s. | n.s. | n.s. |
| C16orf61 | 2.2 | n.s. | n.s. | n.s. | n.s. |
| C16orf63 | n.s. | −1.8 | n.s. | n.s. | n.s. |
| C16orf72 | 3.1 | n.s. | n.s. | n.s. | n.s. |
| C16orf80 | 1.9 | n.s. | n.s. | n.s. | n.s. |
| C17orf32 | 2.6 | n.s. | n.s. | n.s. | n.s. |
| C17orf39 | 2.3 | n.s. | n.s. | n.s. | n.s. |
| C17orf44 | n.s. | −2.8 | n.s. | n.s. | n.s. |
| C17orf63 | n.s. | −1.9 | n.s. | n.s. | n.s. |
| C17orf67 | n.s. | n.s. | 2.4 | 1.9 | 2.5 |
| C18orf17 | −2.3 | −1.9 | n.s. | n.s. | n.s. |
| C18orf19 | 2.6 | n.s. | n.s. | n.s. | n.s. |
| C18orf24 | 3.9 | n.s. | n.s. | n.s. | n.s. |
| C18orf25 | 4.7 | n.s. | n.s. | n.s. | n.s. |
| C18orf37 | 2.8 | n.s. | n.s. | n.s. | n.s. |
| C18orf50 | −3.0 | −2.1 | n.s. | n.s. | n.s. |
| C18orf54 | n.s. | −3.2 | n.s. | n.s. | n.s. |
| C18orf56 | −1.8 | n.s. | n.s. | n.s. | n.s. |
| C19orf24 | 1.9 | n.s. | n.s. | n.s. | n.s. |
| C19orf25 | 2.9 | n.s. | n.s. | n.s. | n.s. |
| C19orf28 | 2.7 | n.s. | n.s. | n.s. | n.s. |
| C19orf48 | 2.2 | n.s. | n.s. | n.s. | n.s. |
| C19orf6 | 2.0 | n.s. | n.s. | n.s. | n.s. |
| C19orf61 | n.s. | 1.9 | n.s. | n.s. | n.s. |
| C1GALT1 | 2.0 | n.s. | n.s. | n.s. | n.s. |
| C1orf104 | −1.8 | n.s. | n.s. | n.s. | n.s. |
| C1orf107 | 2.0 | n.s. | n.s. | n.s. | n.s. |
| C1orf108 | 1.9 | n.s. | n.s. | n.s. | n.s. |
| C1orf112 | 2.5 | 1.9 | n.s. | n.s. | n.s. |
| C1orf116 | 7.6 | n.s. | n.s. | n.s. | n.s. |
| C1orf128 | 2.6 | n.s. | n.s. | n.s. | n.s. |
| C1orf133 | −4.0 | n.s. | n.s. | n.s. | n.s. |
| C1orf135 | 8.0 | n.s. | n.s. | n.s. | n.s. |
| C1orf144 | 1.8 | n.s. | −2.1 | −1.9 | n.s. |
| C1orf163 | 2.3 | n.s. | n.s. | n.s. | n.s. |
| C1orf165 | −3.5 | n.s. | n.s. | n.s. | n.s. |
| C1orf175 | 1.9 | n.s. | n.s. | n.s. | n.s. |
| C1orf192 | −2.1 | n.s. | n.s. | n.s. | n.s. |
| C1orf198 | −2.9 | n.s. | n.s. | n.s. | n.s. |
| C1orf201 | 2.0 | n.s. | n.s. | n.s. | n.s. |
| C1orf21 | −2.1 | n.s. | n.s. | n.s. | n.s. |
| C1orf213 | −2.7 | −2.0 | n.s. | n.s. | n.s. |
| C1orf216 | n.s. | n.s. | −2.0 | n.s. | n.s. |
| C1orf217 | n.s. | 2.0 | n.s. | n.s. | n.s. |
| C1orf25 | −2.0 | −2.5 | n.s. | n.s. | n.s. |
| C1orf43 | 1.9 | n.s. | n.s. | n.s. | n.s. |
| C1orf46 | 5.1 | 3.4 | n.s. | n.s. | n.s. |
| C1orf51 | 2.1 | n.s. | n.s. | n.s. | n.s. |
| C1orf55 | 4.9 | n.s. | n.s. | n.s. | n.s. |
| C1orf56 | −8.0 | n.s. | n.s. | n.s. | n.s. |
| C1orf58 | 2.1 | −1.9 | n.s. | n.s. | n.s. |
| C1orf63 | −2.5 | n.s. | n.s. | n.s. | n.s. |
| C1orf69 | 2.2 | n.s. | n.s. | n.s. | n.s. |
| C1orf71 | −2.0 | n.s. | n.s. | n.s. | n.s. |
| C1orf75 | 2.5 | n.s. | n.s. | n.s. | n.s. |
| C1orf77 | 1.8 | n.s. | n.s. | n.s. | n.s. |
| C1orf9 | 2.5 | n.s. | n.s. | n.s. | n.s. |
| C1orf93 | 1.9 | n.s. | n.s. | n.s. | n.s. |
| C1orf96 | n.s. | −2.0 | n.s. | n.s. | −2.4 |
| C1QTNF4 | n.s. | n.s. | n.s. | −2.3 | n.s. |
| C1RL | −2.4 | n.s. | n.s. | n.s. | n.s. |
| C1S | −2.8 | −2.4 | n.s. | n.s. | n.s. |
| C20orf108 | −2.0 | n.s. | n.s. | n.s. | n.s. |
| C20orf111 | 2.1 | n.s. | n.s. | n.s. | n.s. |
| C20orf165 | n.s. | n.s. | −2.3 | n.s. | n.s. |
| C20orf177 | −1.9 | −3.3 | n.s. | n.s. | n.s. |
| C20orf19 | −2.1 | n.s. | n.s. | n.s. | n.s. |
| C20orf198 | 1.9 | n.s. | n.s. | n.s. | n.s. |
| C20orf23 | 7.9 | n.s. | n.s. | n.s. | n.s. |
| C20orf3 | n.s. | n.s. | −2.0 | n.s. | n.s. |
| C20orf59 | 2.0 | n.s. | n.s. | n.s. | n.s. |
| C20orf7 | 1.9 | n.s. | n.s. | n.s. | n.s. |
| C20orf74 | n.s. | n.s. | 2.5 | n.s. | n.s. |
| C21orf114 | 1.9 | n.s. | n.s. | n.s. | n.s. |
| C21orf2 | n.s. | n.s. | −1.8 | n.s. | n.s. |
| C21orf34 | −2.1 | n.s. | n.s. | n.s. | n.s. |
| C21orf45 | 2.3 | n.s. | n.s. | n.s. | n.s. |
| C21orf51 | −1.8 | −1.9 | n.s. | n.s. | n.s. |
| C21orf74 | 1.9 | n.s. | n.s. | n.s. | n.s. |
| C21orf86 | n.s. | −2.9 | n.s. | n.s. | n.s. |
| C21orf91 | −1.9 | n.s. | n.s. | n.s. | n.s. |
| C22orf24 | n.s. | n.s. | n.s. | 1.9 | n.s. |
| C22orf9 | −2.7 | −2.0 | n.s. | n.s. | n.s. |
| C2orf18 | 1.9 | n.s. | n.s. | n.s. | n.s. |
| C2orf3 | −2.3 | −1.9 | n.s. | n.s. | n.s. |
| C2orf34 | −2.5 | n.s. | n.s. | n.s. | n.s. |
| C2orf49 | −2.2 | n.s. | n.s. | n.s. | n.s. |
| C2orf60 | −1.9 | n.s. | n.s. | n.s. | n.s. |
| C2orf7 | 2.8 | n.s. | n.s. | n.s. | n.s. |
| C3 | n.s. | n.s. | n.s. | −2.5 | n.s. |
| C3orf26 | 1.8 | n.s. | n.s. | n.s. | n.s. |
| C3orf33 | −1.8 | n.s. | n.s. | n.s. | n.s. |
| C3orf34 | −1.9 | −5.5 | n.s. | n.s. | n.s. |
| C3orf52 | 3.7 | 3.8 | n.s. | n.s. | n.s. |
| C3orf63 | −3.1 | −1.9 | n.s. | n.s. | n.s. |
| C4orf12 | −2.5 | −2.2 | n.s. | n.s. | n.s. |
| C4orf15 | 2.5 | n.s. | n.s. | n.s. | n.s. |
| C4orf23 | n.s. | n.s. | −1.8 | n.s. | n.s. |
| C4orf32 | n.s. | n.s. | n.s. | n.s. | 1.8 |
| C4orf34 | n.s. | −2.0 | n.s. | n.s. | n.s. |
| C5orf13 | −2.0 | n.s. | n.s. | n.s. | n.s. |
| C5orf16 | 2.1 | n.s. | n.s. | n.s. | n.s. |
| C5orf23 | n.s. | 2.1 | n.s. | n.s. | n.s. |
| C5orf24 | 2.3 | n.s. | n.s. | n.s. | n.s. |
| C5orf34 | 2.4 | n.s. | n.s. | n.s. | n.s. |
| C5orf4 | −1.8 | −1.8 | n.s. | n.s. | n.s. |
| C6orf105 | 6.4 | 5.0 | n.s. | n.s. | n.s. |
| C6orf107 | −1.9 | n.s. | n.s. | n.s. | n.s. |
| C6orf117 | 2.9 | n.s. | n.s. | n.s. | n.s. |
| C6orf128 | 2.0 | n.s. | n.s. | n.s. | n.s. |
| C6orf129 | 2.5 | n.s. | n.s. | n.s. | n.s. |
| C6orf130 | n.s. | −2.0 | n.s. | n.s. | n.s. |
| C6orf151 | 2.3 | n.s. | n.s. | n.s. | n.s. |
| C6orf155 | −4.2 | n.s. | n.s. | n.s. | n.s. |
| C6orf166 | 2.4 | n.s. | n.s. | n.s. | n.s. |
| C6orf170 | −2.7 | −2.9 | n.s. | n.s. | n.s. |
| C6orf173 | 4.2 | n.s. | n.s. | n.s. | n.s. |
| C6orf199 | −2.0 | −2.3 | n.s. | n.s. | n.s. |
| C6orf204 | n.s. | −2.1 | n.s. | n.s. | −1.9 |
| C6orf206 | −8.2 | n.s. | n.s. | n.s. | n.s. |
| C6orf32 | −3.6 | n.s. | n.s. | n.s. | n.s. |
| C6orf5 | n.s. | n.s. | n.s. | n.s. | 2.8 |
| C6orf62 | −2.0 | −2.6 | n.s. | n.s. | n.s. |
| C6orf65 | −4.0 | −2.0 | n.s. | n.s. | n.s. |
| C6orf66 | 1.8 | n.s. | n.s. | n.s. | n.s. |
| C6orf85 | 2.4 | n.s. | n.s. | n.s. | n.s. |
| C6orf89 | −2.0 | −1.8 | n.s. | n.s. | n.s. |
| C7orf10 | 5.7 | n.s. | n.s. | n.s. | n.s. |
| C7orf19 | −2.8 | −2.1 | n.s. | n.s. | n.s. |
| C7orf20 | 2.2 | n.s. | n.s. | n.s. | n.s. |
| C7orf24 | n.s. | 1.9 | n.s. | n.s. | n.s. |
| C7orf29 | n.s. | n.s. | n.s. | n.s. | 4.6 |
| C7orf38 | n.s. | −2.2 | n.s. | n.s. | n.s. |
| C7orf40 | 4.0 | 2.6 | n.s. | n.s. | n.s. |
| C7orf41 | −2.7 | −3.0 | n.s. | n.s. | n.s. |
| C7orf51 | n.s. | n.s. | n.s. | n.s. | −2.9 |
| C7orf53 | 3.1 | n.s. | n.s. | n.s. | n.s. |
| C7orf55 | n.s. | −1.8 | n.s. | n.s. | n.s. |
| C8orf31 | −2.0 | n.s. | n.s. | n.s. | n.s. |
| C8orf4 | 1.9 | n.s. | n.s. | n.s. | n.s. |
| C8orf47 | n.s. | −2.2 | n.s. | n.s. | n.s. |
| C8orf61 | n.s. | n.s. | 1.9 | 2.1 | 2.0 |
| C8orf66 | n.s. | n.s. | 2.5 | n.s. | n.s. |
| C9orf100 | 1.8 | n.s. | n.s. | n.s. | n.s. |
| C9orf127 | −1.9 | n.s. | n.s. | n.s. | n.s. |
| C9orf130 | n.s. | −1.9 | n.s. | n.s. | n.s. |
| C9orf139 | n.s. | n.s. | n.s. | −2.7 | n.s. |
| C9orf140 | n.s. | −2.7 | n.s. | n.s. | n.s. |
| C9orf151 | 2.0 | n.s. | n.s. | n.s. | n.s. |
| C9orf152 | 2.9 | n.s. | n.s. | n.s. | n.s. |
| C9orf32 | 2.7 | n.s. | n.s. | n.s. | n.s. |
| C9orf39 | −3.9 | n.s. | n.s. | n.s. | n.s. |
| C9orf44 | 2.1 | n.s. | n.s. | n.s. | n.s. |
| C9orf5 | n.s. | −1.9 | n.s. | n.s. | n.s. |
| C9orf52 | −3.0 | n.s. | n.s. | n.s. | n.s. |
| C9orf53 | n.s. | n.s. | 1.9 | n.s. | n.s. |
| C9orf72 | 1.9 | n.s. | n.s. | n.s. | n.s. |
| C9orf85 | −2.2 | n.s. | n.s. | n.s. | n.s. |
| C9orf89 | 2.1 | n.s. | n.s. | n.s. | n.s. |
| CA13 | n.s. | n.s. | n.s. | n.s. | 2.1 |
| CA503034 | n.s. | n.s. | n.s. | 4.3 | n.s. |
| CA5B | −3.8 | −4.1 | n.s. | n.s. | n.s. |
| CA772440 | n.s. | n.s. | n.s. | 1.9 | n.s. |
| CA843452 | n.s. | n.s. | 2.7 | 2.8 | n.s. |
| CA866957 | n.s. | n.s. | n.s. | 1.9 | n.s. |
| CABC1 | n.s. | −2.1 | n.s. | n.s. | n.s. |
| CABYR | n.s. | −2.1 | n.s. | n.s. | n.s. |
| CACHD1 | −1.8 | n.s. | n.s. | n.s. | n.s. |
| CACNA1A | 7.1 | n.s. | n.s. | n.s. | n.s. |
| CACNA1C | −2.6 | n.s. | n.s. | n.s. | n.s. |
| CACNA2D1 | n.s. | n.s. | n.s. | n.s. | 2.3 |
| CADM1 | −1.9 | −2.1 | n.s. | n.s. | n.s. |
| CADPS2 | −2.1 | n.s. | n.s. | n.s. | n.s. |
| CALB2 | 7.8 | n.s. | n.s. | n.s. | n.s. |
| CALD1 | −2.0 | n.s. | n.s. | n.s. | n.s. |
| CALML4 | 2.8 | n.s. | n.s. | n.s. | n.s. |
| CAMK1 | n.s. | n.s. | −1.9 | n.s. | n.s. |
| CAMK2D | n.s. | 2.6 | n.s. | n.s. | n.s. |
| CAMK2G | −2.1 | n.s. | n.s. | n.s. | n.s. |
| CAMKK1 | n.s. | n.s. | −1.9 | n.s. | n.s. |
| CAND2 | −4.6 | n.s. | n.s. | n.s. | n.s. |
| CANT1 | 2.0 | n.s. | n.s. | n.s. | n.s. |
| CAPG | −1.9 | n.s. | n.s. | n.s. | n.s. |
| CAPS | −3.3 | −2.9 | n.s. | n.s. | n.s. |
| CAPS2 | −5.0 | n.s. | n.s. | n.s. | n.s. |
| CAPZA1 | −13.4 | n.s. | n.s. | n.s. | n.s. |
| CAPZB | n.s. | 1.9 | n.s. | n.s. | n.s. |
| CARD10 | −3.0 | n.s. | n.s. | n.s. | n.s. |
| CARD8 | −1.9 | −2.0 | n.s. | n.s. | n.s. |
| CARD9 | n.s. | 2.1 | n.s. | n.s. | n.s. |
| CARKL | 1.8 | n.s. | n.s. | n.s. | n.s. |
| CASC2 | n.s. | n.s. | 4.0 | n.s. | n.s. |
| CASC4 | −1.8 | n.s. | n.s. | n.s. | n.s. |
| CASC5 | n.s. | −2.1 | n.s. | 2.2 | n.s. |
| CASD1 | −2.1 | n.s. | n.s. | n.s. | n.s. |
| CASP1 | −2.4 | n.s. | n.s. | n.s. | n.s. |
| CAST | −2.2 | n.s. | n.s. | n.s. | n.s. |
| CAT | −1.9 | −3.5 | n.s. | n.s. | n.s. |
| CAV2 | −2.1 | n.s. | n.s. | n.s. | n.s. |
| CB984746 | n.s. | n.s. | 1.8 | n.s. | n.s. |
| CBFA2T2 | n.s. | −1.9 | n.s. | n.s. | n.s. |
| CBFB | 2.3 | n.s. | n.s. | n.s. | n.s. |
| CBLL1 | 2.4 | n.s. | n.s. | n.s. | n.s. |
| CBLN3 | −2.3 | −2.0 | n.s. | n.s. | n.s. |
| CBR4 | −1.9 | n.s. | n.s. | n.s. | n.s. |
| CBS | −2.0 | n.s. | n.s. | n.s. | n.s. |
| CBX4 | 2.8 | 2.1 | n.s. | n.s. | n.s. |
| CBX5 | 2.2 | n.s. | n.s. | n.s. | n.s. |
| CBX6 | n.s. | −1.9 | n.s. | n.s. | n.s. |
| CC2D2A | −2.0 | n.s. | n.s. | n.s. | n.s. |
| CCBE1 | −2.0 | n.s. | n.s. | n.s. | n.s. |
| CCDC102A | −10.2 | n.s. | n.s. | n.s. | n.s. |
| CCDC113 | −2.3 | n.s. | n.s. | n.s. | n.s. |
| CCDC115 | n.s. | −2.1 | n.s. | n.s. | n.s. |
| CCDC123 | 3.6 | n.s. | n.s. | n.s. | n.s. |
| CCDC124 | 2.0 | n.s. | n.s. | n.s. | n.s. |
| CCDC13 | 3.1 | n.s. | n.s. | n.s. | n.s. |
| CCDC131 | −2.0 | n.s. | n.s. | n.s. | n.s. |
| CCDC134 | 3.1 | n.s. | n.s. | n.s. | n.s. |
| CCDC136 | −1.9 | n.s. | n.s. | n.s. | n.s. |
| CCDC137 | 2.2 | n.s. | n.s. | n.s. | n.s. |
| CCDC15 | 2.3 | n.s. | n.s. | n.s. | n.s. |
| CCDC16 | n.s. | −1.8 | n.s. | n.s. | n.s. |
| CCDC18 | −4.1 | n.s. | n.s. | n.s. | n.s. |
| CCDC19 | n.s. | n.s. | −2.9 | n.s. | n.s. |
| CCDC32 | −2.0 | −2.2 | n.s. | n.s. | n.s. |
| CCDC34 | n.s. | −3.3 | n.s. | n.s. | n.s. |
| CCDC50 | −2.7 | n.s. | n.s. | n.s. | n.s. |
| CCDC69 | n.s. | n.s. | −2.1 | −2.4 | −2.2 |
| CCDC74B | −2.8 | n.s. | n.s. | n.s. | n.s. |
| CCDC75 | 3.0 | n.s. | n.s. | n.s. | n.s. |
| CCDC77 | 2.7 | n.s. | n.s. | n.s. | n.s. |
| CCDC85B | n.s. | −2.0 | n.s. | n.s. | n.s. |
| CCDC86 | 2.4 | 2.0 | n.s. | n.s. | n.s. |
| CCDC88A | n.s. | −2.0 | n.s. | n.s. | n.s. |
| CCDC89 | n.s. | −2.2 | n.s. | n.s. | n.s. |
| CCDC91 | n.s. | 2.5 | n.s. | n.s. | n.s. |
| CCDC93 | 2.0 | n.s. | n.s. | n.s. | n.s. |
| CCDC98 | −2.2 | n.s. | n.s. | n.s. | n.s. |
| CCDC99 | 3.2 | n.s. | n.s. | n.s. | n.s. |
| CCK | 3.1 | n.s. | n.s. | n.s. | n.s. |
| CCL2 | n.s. | 4.2 | n.s. | n.s. | n.s. |
| CCL26 | 3.3 | n.s. | n.s. | n.s. | n.s. |
| CCNA2 | 3.2 | n.s. | n.s. | n.s. | n.s. |
| CCNB1 | 2.5 | −1.8 | n.s. | n.s. | n.s. |
| CCNB2 | n.s. | −2.1 | n.s. | n.s. | n.s. |
| CCND1 | n.s. | 2.2 | n.s. | n.s. | n.s. |
| CCND3 | 2.6 | 2.0 | n.s. | n.s. | n.s. |
| CCNE1 | 2.3 | 1.8 | n.s. | n.s. | n.s. |
| CCNE2 | 2.3 | 4.7 | n.s. | n.s. | n.s. |
| CCNF | 2.3 | n.s. | −2.1 | n.s. | n.s. |
| CCNG1 | −2.2 | n.s. | n.s. | n.s. | n.s. |
| CCNG2 | n.s. | −2.2 | n.s. | n.s. | n.s. |
| CCNL2 | −2.1 | n.s. | n.s. | n.s. | n.s. |
| CCPG1 | −2.0 | n.s. | n.s. | n.s. | n.s. |
| CCR2 | n.s. | n.s. | n.s. | −6.0 | n.s. |
| CCRL1 | −5.5 | n.s. | n.s. | n.s. | n.s. |
| CCRN4L | 2.4 | n.s. | n.s. | n.s. | n.s. |
| CCT5 | 2.4 | 2.3 | n.s. | n.s. | n.s. |
| CD109 | −2.6 | −2.4 | n.s. | n.s. | n.s. |
| CD1C | n.s. | n.s. | n.s. | n.s. | 6.3 |
| CD24 | 5.1 | n.s. | n.s. | n.s. | n.s. |
| CD274 | 3.1 | 4.6 | n.s. | n.s. | n.s. |
| CD276 | 1.8 | n.s. | n.s. | n.s. | n.s. |
| CD28 | n.s. | n.s. | 4.1 | n.s. | n.s. |
| CD302 | −2.8 | n.s. | n.s. | n.s. | n.s. |
| CD34 | n.s. | n.s. | n.s. | n.s. | 2.8 |
| CD3EAP | n.s. | 2.4 | n.s. | n.s. | n.s. |
| CD44 | 2.2 | 1.8 | n.s. | n.s. | n.s. |
| CD47 | −2.3 | n.s. | n.s. | n.s. | n.s. |
| CD55 | 2.0 | 2.3 | n.s. | n.s. | n.s. |
| CD69 | 11.5 | n.s. | n.s. | n.s. | n.s. |
| CD9 | −3.2 | −1.9 | n.s. | n.s. | n.s. |
| CDA | 2.7 | n.s. | n.s. | n.s. | n.s. |
| CDA08 | −2.1 | n.s. | n.s. | n.s. | n.s. |
| CDC14B | −2.0 | 3.1 | n.s. | n.s. | n.s. |
| CDC2 | 6.1 | n.s. | n.s. | n.s. | n.s. |
| CDC20 | 3.2 | n.s. | n.s. | n.s. | n.s. |
| CDC23 | 2.0 | n.s. | n.s. | n.s. | n.s. |
| CDC25A | 7.5 | 8.0 | n.s. | n.s. | n.s. |
| CDC25C | 4.6 | −2.0 | n.s. | n.s. | n.s. |
| CDC2L1 | n.s. | n.s. | n.s. | −1.9 | −1.9 |
| CDC2L5 | −1.8 | n.s. | n.s. | n.s. | n.s. |
| CDC2L6 | 3.0 | −2.3 | n.s. | 3.8 | n.s. |
| CDC42BPA | −2.8 | n.s. | n.s. | n.s. | n.s. |
| CDC42EP1 | n.s. | 2.3 | n.s. | n.s. | n.s. |
| CDC42EP2 | −3.2 | n.s. | n.s. | n.s. | n.s. |
| CDC42EP3 | −1.9 | −2.0 | n.s. | n.s. | n.s. |
| CDC45L | 6.3 | 3.2 | n.s. | n.s. | n.s. |
| CDC6 | 3.6 | 3.7 | n.s. | n.s. | n.s. |
| CDCA2 | 3.1 | n.s. | n.s. | n.s. | n.s. |
| CDCA3 | 4.1 | −2.2 | n.s. | n.s. | n.s. |
| CDCA4 | 3.1 | 2.1 | n.s. | n.s. | n.s. |
| CDCA5 | 4.8 | n.s. | n.s. | n.s. | n.s. |
| CDCA7 | n.s. | 2.2 | 2.0 | n.s. | 1.9 |
| CDCA8 | 4.8 | n.s. | n.s. | n.s. | n.s. |
| CDCP1 | 4.5 | 7.1 | n.s. | n.s. | n.s. |
| CDH11 | −2.0 | n.s. | n.s. | n.s. | n.s. |
| CDH4 | n.s. | 2.4 | n.s. | n.s. | n.s. |
| CDH7 | n.s. | n.s. | 5.0 | n.s. | 4.9 |
| CDIPT | n.s. | n.s. | −1.9 | n.s. | n.s. |
| CDK2 | 2.4 | n.s. | n.s. | n.s. | n.s. |
| CDK2AP2 | 1.9 | n.s. | n.s. | n.s. | n.s. |
| CDK3 | −1.9 | n.s. | n.s. | n.s. | n.s. |
| CDK5R1 | 3.4 | 2.0 | n.s. | n.s. | n.s. |
| CDK5RAP2 | 4.4 | n.s. | n.s. | n.s. | n.s. |
| CDK5RAP3 | n.s. | n.s. | −1.9 | n.s. | n.s. |
| CDK6 | −1.9 | n.s. | n.s. | n.s. | n.s. |
| CDKL1 | −4.1 | n.s. | n.s. | n.s. | n.s. |
| CDKN1B | n.s. | −1.8 | n.s. | n.s. | n.s. |
| CDKN1C | n.s. | −2.2 | n.s. | n.s. | n.s. |
| CDKN2C | n.s. | −2.2 | n.s. | n.s. | n.s. |
| CDKN3 | n.s. | −2.9 | n.s. | n.s. | n.s. |
| CDON | −5.4 | −4.4 | n.s. | n.s. | n.s. |
| CDR2L | 1.9 | n.s. | n.s. | n.s. | n.s. |
| CDRT15 | n.s. | n.s. | n.s. | 5.6 | n.s. |
| CDT1 | 3.6 | 2.1 | n.s. | n.s. | n.s. |
| CDYL | 3.1 | n.s. | n.s. | n.s. | n.s. |
| CEBPD | −2.5 | n.s. | n.s. | n.s. | n.s. |
| CECR1 | −3.5 | n.s. | n.s. | n.s. | n.s. |
| CECR2 | n.s. | n.s. | n.s. | n.s. | 2.2 |
| CELSR1 | n.s. | n.s. | 2.0 | 2.5 | 2.2 |
| CENPA | 2.9 | −2.2 | n.s. | n.s. | n.s. |
| CENPE | 2.2 | −1.9 | n.s. | n.s. | n.s. |
| CENPF | 2.5 | n.s. | n.s. | n.s. | n.s. |
| CENPI | 2.7 | n.s. | n.s. | n.s. | n.s. |
| CENPJ | 2.1 | n.s. | n.s. | n.s. | n.s. |
| CENPK | 2.8 | n.s. | n.s. | 1.8 | n.s. |
| CENPL | 2.1 | n.s. | n.s. | n.s. | n.s. |
| CENPM | 3.6 | n.s. | n.s. | n.s. | n.s. |
| CENPN | 3.7 | n.s. | n.s. | n.s. | n.s. |
| CENPO | 3.0 | n.s. | n.s. | n.s. | n.s. |
| CENPQ | 2.9 | n.s. | n.s. | n.s. | n.s. |
| CENPT | 2.2 | n.s. | n.s. | n.s. | n.s. |
| CENTD2 | −2.2 | n.s. | n.s. | n.s. | n.s. |
| CENTG3 | n.s. | n.s. | −1.9 | n.s. | n.s. |
| CEP152 | 2.0 | n.s. | n.s. | n.s. | n.s. |
| CEP170 | n.s. | 1.9 | n.s. | n.s. | n.s. |
| CEP290 | n.s. | n.s. | n.s. | n.s. | −2.2 |
| CEP350 | n.s. | −2.3 | n.s. | n.s. | n.s. |
| CEP55 | 3.1 | n.s. | n.s. | n.s. | n.s. |
| CEP57 | −1.9 | n.s. | n.s. | n.s. | n.s. |
| CEP68 | −2.0 | −1.9 | n.s. | n.s. | n.s. |
| CEP70 | −1.9 | −2.6 | n.s. | n.s. | n.s. |
| CEP76 | 2.1 | n.s. | n.s. | n.s. | n.s. |
| CEP78 | 2.2 | n.s. | n.s. | n.s. | n.s. |
| CERK | n.s. | 2.5 | n.s. | n.s. | n.s. |
| CES2 | n.s. | −2.2 | n.s. | n.s. | n.s. |
| CFB | −3.1 | n.s. | n.s. | n.s. | n.s. |
| CFD | −2.0 | n.s. | n.s. | n.s. | n.s. |
| CFDP1 | n.s. | 3.1 | n.s. | n.s. | n.s. |
| CFH | −1.9 | n.s. | n.s. | n.s. | n.s. |
| CFL1 | n.s. | n.s. | −1.9 | n.s. | n.s. |
| CGB | 5.3 | n.s. | n.s. | n.s. | n.s. |
| CGGBP1 | 1.9 | n.s. | n.s. | n.s. | n.s. |
| CGNL1 | n.s. | −2.3 | n.s. | n.s. | −2.0 |
| CH25H | −2.3 | n.s. | n.s. | n.s. | n.s. |
| CHAC2 | 3.8 | 2.5 | n.s. | n.s. | n.s. |
| CHAF1A | 4.1 | n.s. | n.s. | n.s. | n.s. |
| CHAF1B | 2.3 | n.s. | n.s. | n.s. | n.s. |
| CHD6 | −1.9 | n.s. | n.s. | n.s. | n.s. |
| CHDH | −5.2 | n.s. | n.s. | n.s. | n.s. |
| CHKA | 2.3 | n.s. | n.s. | n.s. | n.s. |
| CHM | 2.0 | n.s. | n.s. | n.s. | n.s. |
| CHMP1B | 2.2 | n.s. | n.s. | n.s. | n.s. |
| CHN1 | −1.9 | n.s. | n.s. | n.s. | n.s. |
| CHP | n.s. | −2.0 | n.s. | n.s. | n.s. |
| CHRNA9 | 5.4 | n.s. | n.s. | n.s. | n.s. |
| CHST11 | 2.8 | n.s. | n.s. | n.s. | n.s. |
| CHST2 | n.s. | 1.9 | n.s. | n.s. | n.s. |
| CHST3 | 2.0 | n.s. | n.s. | n.s. | n.s. |
| CHST6 | 2.0 | n.s. | n.s. | n.s. | n.s. |
| CHSY-2 | n.s. | 4.1 | n.s. | n.s. | n.s. |
| CHTF18 | 3.4 | n.s. | n.s. | n.s. | n.s. |
| CILP | n.s. | n.s. | 2.2 | n.s. | n.s. |
| CINP | n.s. | n.s. | n.s. | n.s. | 2.0 |
| CIP29 | 2.2 | n.s. | n.s. | n.s. | n.s. |
| CIR | n.s. | −1.9 | n.s. | n.s. | n.s. |
| CIRBP | −5.1 | −2.5 | n.s. | n.s. | n.s. |
| CITED2 | −2.1 | n.s. | n.s. | n.s. | n.s. |
| CITED4 | n.s. | n.s. | n.s. | −7.5 | n.s. |
| CK818527 | n.s. | n.s. | n.s. | 2.3 | n.s. |
| CKAP2 | 1.9 | −1.9 | n.s. | n.s. | n.s. |
| CKAP2L | 3.8 | n.s. | n.s. | n.s. | n.s. |
| CKLF | 1.8 | n.s. | n.s. | n.s. | n.s. |
| CKS1B | 3.3 | n.s. | n.s. | n.s. | n.s. |
| CKS2 | 3.2 | n.s. | n.s. | n.s. | n.s. |
| CLCC1 | −4.5 | −3.2 | n.s. | n.s. | n.s. |
| CLCF1 | 2.8 | 2.0 | n.s. | n.s. | n.s. |
| CLCN5 | 2.0 | n.s. | n.s. | n.s. | n.s. |
| CLDN11 | −3.7 | −2.0 | n.s. | n.s. | n.s. |
| CLDND1 | 3.2 | n.s. | n.s. | n.s. | n.s. |
| CLEC2B | −3.7 | n.s. | n.s. | n.s. | n.s. |
| CLEC3B | −2.3 | n.s. | n.s. | n.s. | n.s. |
| CLIP1 | n.s. | n.s. | n.s. | −1.9 | −2.2 |
| CLIP3 | −5.9 | n.s. | n.s. | n.s. | n.s. |
| CLIP4 | −2.5 | n.s. | n.s. | n.s. | n.s. |
| CLK4 | −2.3 | −1.9 | n.s. | n.s. | n.s. |
| CLN5 | 2.8 | n.s. | n.s. | n.s. | n.s. |
| CLN6 | 2.7 | n.s. | n.s. | n.s. | n.s. |
| CLN8 | n.s. | −2.1 | n.s. | n.s. | n.s. |
| CLP1 | 1.9 | n.s. | n.s. | n.s. | n.s. |
| CLPB | 2.8 | n.s. | n.s. | n.s. | n.s. |
| CLSPN | 2.5 | n.s. | n.s. | n.s. | n.s. |
| CLTC | 2.7 | n.s. | n.s. | n.s. | n.s. |
| CLU | −2.0 | −1.8 | n.s. | n.s. | n.s. |
| CLUAP1 | −2.0 | −2.0 | n.s. | n.s. | n.s. |
| CMAH | −2.3 | n.s. | n.s. | n.s. | n.s. |
| CMBL | −1.9 | n.s. | n.s. | n.s. | n.s. |
| CMTM7 | 2.1 | 2.2 | n.s. | n.s. | n.s. |
| CNDP2 | n.s. | n.s. | −1.9 | n.s. | n.s. |
| CNIH3 | n.s. | 2.0 | n.s. | n.s. | n.s. |
| CNKSR2 | −5.5 | −3.6 | n.s. | n.s. | n.s. |
| CNKSR3 | −2.2 | n.s. | n.s. | n.s. | n.s. |
| CNN1 | 4.2 | n.s. | n.s. | n.s. | n.s. |
| CNNM2 | 2.2 | n.s. | n.s. | n.s. | n.s. |
| CNOT3 | −1.8 | n.s. | n.s. | n.s. | n.s. |
| CNOT6L | −3.1 | n.s. | n.s. | n.s. | n.s. |
| CNPY3 | n.s. | n.s. | n.s. | n.s. | 2.4 |
| CNTN3 | −5.0 | −2.6 | n.s. | n.s. | n.s. |
| COCH | n.s. | −1.9 | n.s. | n.s. | n.s. |
| COG6 | −2.0 | n.s. | n.s. | n.s. | n.s. |
| COIL | n.s. | n.s. | −2.0 | n.s. | n.s. |
| COL12A1 | −2.3 | −2.2 | n.s. | n.s. | n.s. |
| COL14A1 | −23.5 | n.s. | n.s. | n.s. | n.s. |
| COL15A1 | −2.9 | n.s. | n.s. | n.s. | n.s. |
| COL1A1 | n.s. | −2.0 | n.s. | n.s. | n.s. |
| COL1A2 | −2.6 | n.s. | n.s. | n.s. | n.s. |
| COL27A1 | −2.0 | n.s. | n.s. | n.s. | n.s. |
| COL3A1 | −1.9 | −4.8 | n.s. | −1.9 | n.s. |
| COL4A3BP | −2.0 | −3.0 | n.s. | n.s. | n.s. |
| COL5A2 | −2.0 | n.s. | n.s. | n.s. | n.s. |
| COL6A1 | −1.8 | n.s. | n.s. | n.s. | n.s. |
| COL6A6 | −6.3 | n.s. | n.s. | n.s. | n.s. |
| COL8A1 | −2.6 | n.s. | n.s. | n.s. | n.s. |
| COL9A1 | n.s. | n.s. | −1.9 | n.s. | n.s. |
| COLEC12 | −6.2 | −2.3 | n.s. | n.s. | n.s. |
| COMMD10 | −2.4 | n.s. | n.s. | n.s. | n.s. |
| COMMD6 | n.s. | n.s. | 2.2 | n.s. | n.s. |
| COP1 | −3.0 | n.s. | n.s. | n.s. | n.s. |
| COPG | n.s. | 1.8 | n.s. | n.s. | n.s. |
| COPS7B | −2.5 | n.s. | n.s. | n.s. | n.s. |
| COQ10B | 1.9 | n.s. | n.s. | n.s. | n.s. |
| COTL1 | 5.3 | n.s. | n.s. | n.s. | n.s. |
| COX17 | 2.1 | n.s. | n.s. | n.s. | n.s. |
| CPD | −1.9 | n.s. | n.s. | n.s. | n.s. |
| CPEB1 | 4.5 | n.s. | n.s. | n.s. | n.s. |
| CPEB2 | 1.8 | n.s. | n.s. | n.s. | n.s. |
| CPEB3 | −2.1 | −4.0 | n.s. | n.s. | n.s. |
| CPEB4 | 2.1 | n.s. | n.s. | n.s. | n.s. |
| CPNE4 | n.s. | n.s. | n.s. | 2.0 | n.s. |
| CPSF3L | 2.0 | n.s. | n.s. | n.s. | n.s. |
| CPSF6 | −1.9 | n.s. | n.s. | n.s. | n.s. |
| CPT1C | −2.1 | n.s. | n.s. | n.s. | n.s. |
| CR598370 | n.s. | n.s. | n.s. | n.s. | 2.0 |
| CR605947 | n.s. | n.s. | n.s. | 3.1 | 3.2 |
| CR616772 | n.s. | n.s. | −1.8 | n.s. | n.s. |
| CR617865 | n.s. | n.s. | 2.5 | n.s. | n.s. |
| CR740121 | n.s. | n.s. | 1.9 | 2.0 | n.s. |
| CRABP2 | −2.5 | n.s. | n.s. | n.s. | n.s. |
| CRBN | −1.9 | −1.9 | n.s. | n.s. | n.s. |
| CREB1 | −1.8 | −2.0 | n.s. | n.s. | n.s. |
| CREB3L2 | 3.2 | n.s. | n.s. | n.s. | n.s. |
| CREBBP | n.s. | −2.4 | n.s. | n.s. | n.s. |
| CREBZF | −2.8 | −2.1 | n.s. | n.s. | n.s. |
| CREG2 | n.s. | n.s. | 5.3 | n.s. | n.s. |
| CREM | 2.0 | n.s. | n.s. | n.s. | n.s. |
| CRIM1 | −1.9 | n.s. | n.s. | n.s. | n.s. |
| CRIPAK | −2.3 | −2.3 | n.s. | n.s. | n.s. |
| CRISPLD2 | 2.9 | n.s. | n.s. | n.s. | n.s. |
| CRKRS | −1.9 | −1.9 | n.s. | n.s. | n.s. |
| CROCCL2 | n.s. | n.s. | n.s. | −2.5 | n.s. |
| CRSP8 | 2.2 | n.s. | n.s. | n.s. | n.s. |
| CRTC2 | n.s. | 1.8 | n.s. | n.s. | n.s. |
| CRY1 | n.s. | n.s. | n.s. | 1.8 | 2.1 |
| CRY2 | n.s. | n.s. | n.s. | −1.8 | n.s. |
| CRYZ | −1.9 | n.s. | n.s. | n.s. | n.s. |
| CRYZL1 | n.s. | −1.9 | n.s. | n.s. | n.s. |
| CSE1L | 2.1 | n.s. | n.s. | n.s. | n.s. |
| CSF2RB | 1.9 | n.s. | n.s. | n.s. | n.s. |
| CSNK1E | 3.9 | 1.9 | n.s. | n.s. | n.s. |
| CSPG4 | 2.1 | 2.4 | n.s. | n.s. | n.s. |
| CSRP1 | 2.8 | 2.3 | −1.8 | n.s. | n.s. |
| CSRP2 | −2.0 | n.s. | n.s. | n.s. | n.s. |
| CST6 | 2.1 | n.s. | n.s. | n.s. | n.s. |
| CSTA | n.s. | n.s. | n.s. | n.s. | 1.8 |
| CTA-126B4.3 | 2.3 | n.s. | n.s. | n.s. | n.s. |
| CTB- | 1.8 | n.s. | n.s. | n.s. | n.s. |
| 1048E9.5 | | | | | |
| CTBS | −2.5 | n.s. | n.s. | n.s. | n.s. |
| CTDSP2 | 2.1 | n.s. | n.s. | n.s. | n.s. |
| CTDSPL | −2.1 | n.s. | n.s. | n.s. | n.s. |
| CTDSPL2 | −2.1 | n.s. | n.s. | n.s. | n.s. |
| CTGF | n.s. | 2.7 | n.s. | n.s. | n.s. |
| CTNS | 2.8 | n.s. | n.s. | n.s. | n.s. |
| CTPS | 2.8 | 3.4 | n.s. | n.s. | n.s. |
| CTSC | −2.0 | n.s. | n.s. | n.s. | n.s. |
| CTSD | 2.2 | n.s. | n.s. | n.s. | n.s. |
| CTSF | −1.9 | n.s. | n.s. | n.s. | n.s. |
| CTSO | −2.0 | n.s. | n.s. | n.s. | n.s. |
| CTSS | −9.7 | −10.3 | n.s. | n.s. | n.s. |
| CTTN | n.s. | −2.2 | n.s. | n.s. | n.s. |
| CUEDC2 | n.s. | n.s. | −1.9 | n.s. | n.s. |
| CUGBP1 | 2.2 | n.s. | n.s. | n.s. | n.s. |
| CUGBP2 | −2.5 | −2.2 | n.s. | n.s. | n.s. |
| CUL4A | 2.7 | n.s. | n.s. | n.s. | n.s. |
| CUL5 | −1.9 | n.s. | n.s. | n.s. | n.s. |
| CUL7 | −3.0 | n.s. | n.s. | n.s. | n.s. |
| CUTC | n.s. | −1.9 | n.s. | n.s. | n.s. |
| CV326037 | n.s. | n.s. | n.s. | n.s. | 2.7 |
| CXCL2 | 2.6 | n.s. | n.s. | n.s. | n.s. |
| CXCL3 | 3.6 | 3.8 | n.s. | n.s. | n.s. |
| CXCL5 | 7.3 | 3.0 | n.s. | n.s. | n.s. |
| CXCL6 | 3.8 | 2.2 | n.s. | n.s. | n.s. |
| CXorf23 | −2.0 | n.s. | n.s. | n.s. | n.s. |
| CXorf38 | 2.3 | n.s. | n.s. | n.s. | n.s. |
| CXorf39 | −2.3 | n.s. | n.s. | n.s. | n.s. |
| CXorf45 | −2.1 | n.s. | n.s. | n.s. | n.s. |
| CXorf6 | −7.6 | n.s. | n.s. | n.s. | n.s. |
| CXXC6 | n.s. | n.s. | 4.3 | n.s. | n.s. |
| CYB561D2 | 1.8 | n.s. | n.s. | n.s. | n.s. |
| CYB5D2 | −2.0 | n.s. | n.s. | n.s. | n.s. |
| CYB5R3 | −2.3 | n.s. | n.s. | n.s. | n.s. |
| CYBRD1 | −1.8 | n.s. | n.s. | n.s. | n.s. |
| CYCS | 2.0 | 2.1 | n.s. | n.s. | n.s. |
| CYFIP2 | −2.7 | n.s. | n.s. | n.s. | n.s. |
| CYLD | 2.5 | −2.2 | n.s. | n.s. | n.s. |
| CYP19A1 | 4.1 | n.s. | n.s. | n.s. | n.s. |
| CYP1B1 | −2.7 | −3.8 | n.s. | n.s. | n.s. |
| CYP20A1 | n.s. | n.s. | n.s. | 1.9 | n.s. |
| CYP26B1 | −4.0 | n.s. | n.s. | n.s. | n.s. |
| CYP2C18 | 6.7 | n.s. | n.s. | n.s. | n.s. |
| CYP2U1 | −2.5 | n.s. | n.s. | n.s. | n.s. |
| CYP4V2 | −2.8 | n.s. | n.s. | n.s. | n.s. |
| CYR61 | −2.9 | n.s. | n.s. | n.s. | n.s. |
| CYS1 | −11.0 | −2.9 | n.s. | n.s. | n.s. |
| CYYR1 | −3.8 | n.s. | n.s. | n.s. | n.s. |
| D15Wsu75e | 1.8 | n.s. | n.s. | n.s. | n.s. |
| D31825 | n.s. | n.s. | n.s. | n.s. | −6.4 |
| D90075 | n.s. | n.s. | −6.4 | n.s. | n.s. |
| DAAM1 | −2.2 | −2.1 | n.s. | n.s. | n.s. |
| DAAM2 | −4.5 | n.s. | n.s. | n.s. | n.s. |
| DAB2 | −2.2 | n.s. | n.s. | n.s. | n.s. |
| DACT1 | 3.1 | n.s. | n.s. | n.s. | n.s. |
| DAGLA | n.s. | n.s. | 2.1 | 2.4 | 2.7 |
| DAGLB | 2.0 | n.s. | n.s. | n.s. | n.s. |
| DALRD3 | −1.8 | n.s. | n.s. | n.s. | n.s. |
| DAP | n.s. | 3.0 | n.s. | n.s. | n.s. |
| DAPK2 | −3.2 | n.s. | n.s. | n.s. | n.s. |
| DB318193 | n.s. | n.s. | 1.9 | n.s. | n.s. |
| DB318210 | n.s. | n.s. | n.s. | 1.8 | n.s. |
| DB352368 | n.s. | n.s. | 1.9 | n.s. | n.s. |
| DBF4 | 2.3 | n.s. | n.s. | n.s. | n.s. |
| DBNL | n.s. | n.s. | −1.9 | n.s. | n.s. |
| DBP | −2.2 | −2.9 | n.s. | n.s. | n.s. |
| DBT | −2.4 | −4.2 | n.s. | n.s. | n.s. |
| DCBLD1 | 2.4 | n.s. | n.s. | n.s. | n.s. |
| DCBLD2 | 3.0 | n.s. | n.s. | n.s. | n.s. |
| DCHS1 | −4.4 | n.s. | n.s. | n.s. | n.s. |
| DCK | 2.6 | n.s. | n.s. | n.s. | n.s. |
| DCLK1 | n.s. | 2.2 | n.s. | n.s. | n.s. |
| DCLK3 | n.s. | n.s. | −5.9 | n.s. | n.s. |
| DCLRE1B | 2.3 | 2.3 | n.s. | n.s. | n.s. |
| DCN | −2.1 | −3.3 | n.s. | n.s. | n.s. |
| DCP_1_7 | n.s. | n.s. | n.s. | n.s. | 1.8 |
| DCP_22_0 | n.s. | n.s. | 11.0 | 11.7 | 12.0 |
| DCP_22_2 | n.s. | n.s. | 10.4 | 11.0 | 11.8 |
| DCP_22_4 | n.s. | n.s. | 11.0 | 11.5 | 12.3 |
| DCP_22_6 | n.s. | n.s. | 10.6 | 11.5 | 12.4 |
| DCP_22_7 | n.s. | n.s. | 10.7 | 11.3 | 11.3 |
| DCP_22_9 | n.s. | n.s. | 3.0 | 4.0 | 2.9 |
| DCP1A | n.s. | −1.8 | n.s. | n.s. | n.s. |
| DCP1B | n.s. | −1.9 | n.s. | n.s. | n.s. |
| DCTN5 | 2.3 | n.s. | n.s. | n.s. | n.s. |
| DDA1 | 1.9 | n.s. | n.s. | n.s. | n.s. |
| DDAH1 | −2.0 | 2.9 | n.s. | n.s. | n.s. |
| DDAH2 | −2.2 | −2.9 | n.s. | n.s. | n.s. |
| DDB2 | n.s. | −1.9 | n.s. | n.s. | n.s. |
| DDEF1IT1 | n.s. | 2.8 | n.s. | n.s. | n.s. |
| DDEF2 | −1.8 | n.s. | n.s. | n.s. | n.s. |
| DDEFL1 | −2.2 | n.s. | n.s. | n.s. | n.s. |
| DDHD1 | n.s. | n.s. | n.s. | n.s. | −2.0 |
| DDHD2 | −2.0 | n.s. | n.s. | n.s. | n.s. |
| DDI2 | 3.5 | 1.9 | n.s. | n.s. | n.s. |
| DDIT4L | −2.0 | −2.1 | n.s. | n.s. | n.s. |
| DDOST | −2.7 | n.s. | n.s. | n.s. | n.s. |
| DDR1 | 2.1 | n.s. | n.s. | n.s. | n.s. |
| DDR2 | −2.3 | −2.1 | n.s. | n.s. | n.s. |
| DDX17 | −1.8 | n.s. | n.s. | n.s. | n.s. |
| DDX19A | n.s. | 1.9 | n.s. | n.s. | n.s. |
| DDX21 | n.s. | 2.3 | n.s. | n.s. | n.s. |
| DDX24 | 2.1 | n.s. | n.s. | n.s. | n.s. |
| DDX39 | 2.5 | n.s. | n.s. | n.s. | n.s. |
| DDX3X | 2.0 | n.s. | n.s. | n.s. | n.s. |
| DDX46 | −4.3 | −2.4 | n.s. | n.s. | n.s. |
| DDX50 | 2.6 | 2.6 | n.s. | n.s. | n.s. |
| DDX51 | n.s. | 1.9 | n.s. | n.s. | n.s. |
| DDX54 | n.s. | n.s. | −1.8 | n.s. | n.s. |
| DDX55 | 1.9 | n.s. | n.s. | n.s. | n.s. |
| DEDD2 | 2.5 | n.s. | n.s. | n.s. | n.s. |
| DENND1A | −1.8 | n.s. | n.s. | n.s. | n.s. |
| DENND1B | n.s. | n.s. | n.s. | n.s. | −2.0 |
| DENND2A | −5.2 | n.s. | n.s. | n.s. | n.s. |
| DENND2D | −2.7 | n.s. | n.s. | n.s. | n.s. |
| DENND4A | n.s. | n.s. | n.s. | n.s. | −1.8 |
| DENND4C | −2.5 | −3.5 | n.s. | n.s. | n.s. |
| DEPDC1 | 3.2 | −1.8 | n.s. | n.s. | n.s. |
| DEPDC1B | 1.9 | n.s. | n.s. | 1.9 | n.s. |
| DEPDC2 | −4.8 | n.s. | n.s. | n.s. | n.s. |
| DFFA | 1.9 | n.s. | n.s. | n.s. | n.s. |
| DENB59 | −2.9 | −2.0 | n.s. | n.s. | n.s. |
| DGKA | −2.0 | n.s. | −1.9 | n.s. | n.s. |
| DGKE | −2.0 | n.s. | n.s. | n.s. | n.s. |
| DHCR24 | −2.0 | n.s. | n.s. | n.s. | n.s. |
| DHDH | 11.4 | n.s. | n.s. | n.s. | n.s. |
| DHFR | 2.2 | n.s. | n.s. | n.s. | n.s. |
| DHFRL1 | n.s. | −1.9 | n.s. | n.s. | n.s. |
| DHRS1 | −2.5 | n.s. | n.s. | n.s. | n.s. |
| DHRS12 | −2.6 | n.s. | n.s. | n.s. | n.s. |
| DHRS2 | 16.2 | n.s. | n.s. | n.s. | n.s. |
| DHRS7B | 2.5 | n.s. | n.s. | n.s. | n.s. |
| DHTKD1 | −2.2 | n.s. | −2.0 | n.s. | n.s. |
| DHX30 | 2.3 | 2.0 | n.s. | n.s. | n.s. |
| DHX37 | n.s. | 2.2 | n.s. | n.s. | n.s. |
| DHX40 | −1.9 | n.s. | n.s. | n.s. | n.s. |
| DIAPH1 | n.s. | 1.8 | n.s. | n.s. | n.s. |
| DIAPH2 | n.s. | 1.8 | n.s. | n.s. | n.s. |
| DIAPH3 | 2.3 | n.s. | n.s. | −2.9 | n.s. |
| DICER1 | −2.1 | n.s. | n.s. | n.s. | n.s. |
| DIP2A | 1.9 | 1.9 | n.s. | n.s. | n.s. |
| DIRAS3 | 29.4 | 4.7 | n.s. | n.s. | n.s. |
| DIS3L | −1.9 | n.s. | n.s. | n.s. | n.s. |
| DISP1 | 2.2 | n.s. | n.s. | n.s. | n.s. |
| DKFZp434A0530 | −2.2 | n.s. | n.s. | n.s. | n.s. |
| DKFZP434B0335 | −2.0 | n.s. | n.s. | n.s. | n.s. |
| DKFZp434C198 | −2.1 | n.s. | n.s. | n.s. | n.s. |
| DKFZp434D193 | 2.5 | n.s. | n.s. | n.s. | n.s. |
| DKFZp434G0514 | 2.4 | n.s. | n.s. | n.s. | n.s. |
| DKFZp434H0350 | n.s. | 2.6 | n.s. | n.s. | n.s. |
| DKFZp434H1419 | n.s. | −1.9 | n.s. | n.s. | n.s. |
| DKFZp434H152 | 2.2 | 2.0 | n.s. | n.s. | n.s. |
| DKFZp434I062 | 2.2 | 2.2 | n.s. | n.s. | n.s. |
| DKFZP434I0714 | n.s. | −2.6 | n.s. | n.s. | n.s. |
| DKFZp434J1521 | n.s. | 2.1 | n.s. | n.s. | n.s. |
| DKFZp434L1123 | 1.9 | n.s. | n.s. | n.s. | n.s. |
| DKFZp434N1010 | 2.2 | n.s. | n.s. | n.s. | n.s. |
| DKFZp434N2435 | −2.4 | n.s. | n.s. | n.s. | n.s. |
| DKFZp434P1735 | −5.9 | n.s. | n.s. | n.s. | n.s. |
| DKFZP434P211 | n.s. | n.s. | 2.5 | 5.7 | 2.4 |
| DKFZp451A211 | 3.7 | n.s. | n.s. | n.s. | n.s. |
| DKFZp547E087 | 2.1 | n.s. | n.s. | n.s. | n.s. |
| DKFZp564A063 | 2.9 | n.s. | n.s. | n.s. | n.s. |
| DKFZp564C0482 | 2.4 | n.s. | n.s. | n.s. | n.s. |
| DKFZp564F1862 | 2.4 | n.s. | n.s. | n.s. | n.s. |
| DKFZP564J0863 | 1.9 | n.s. | n.s. | n.s. | n.s. |
| DKFZp564J1864 | 3.3 | n.s. | n.s. | n.s. | n.s. |
| DKFZp564K2364 | −1.9 | n.s. | n.s. | n.s. | n.s. |
| DKFZp564L2362 | 2.0 | n.s. | n.s. | n.s. | n.s. |
| DKFZP564O0523 | n.s. | −1.8 | n.s. | n.s. | n.s. |
| DKFZp566K1946 | −1.8 | −2.7 | n.s. | n.s. | n.s. |
| DKFZp586H1322 | −2.5 | n.s. | n.s. | n.s. | n.s. |
| DKFZp586J1119 | n.s. | 3.3 | n.s. | n.s. | n.s. |
| DKFZP586K1520 | n.s. | −2.3 | n.s. | n.s. | n.s. |
| DKFZp667E0512 | n.s. | n.s. | n.s. | n.s. | 2.1 |
| DKFZp667G2110 | −1.9 | n.s. | n.s. | n.s. | n.s. |
| DKFZp761C121 | 1.8 | n.s. | n.s. | n.s. | n.s. |
| DKFZp761D221 | −4.4 | n.s. | n.s. | n.s. | n.s. |
| DKFZp761F0123 | n.s. | 2.1 | n.s. | n.s. | n.s. |
| DKFZp761I172 | −1.9 | n.s. | n.s. | n.s. | n.s. |
| DKFZp761J17121 | −1.8 | n.s. | n.s. | n.s. | n.s. |
| DKFZp761M0423 | 1.9 | n.s. | n.s. | n.s. | n.s. |
| DKFZP761M1511 | n.s. | −3.5 | n.s. | n.s. | n.s. |
| DKFZp761P0423 | 2.0 | 2.0 | n.s. | n.s. | n.s. |
| DKFZp762B2310 | 2.0 | 1.9 | n.s. | n.s. | n.s. |
| DKK2 | −20.5 | n.s. | n.s. | n.s. | n.s. |
| DLC1 | −2.1 | n.s. | n.s. | n.s. | n.s. |
| DLEU2 | 3.0 | 2.3 | n.s. | n.s. | n.s. |
| DLEU2L | 2.4 | n.s. | n.s. | n.s. | n.s. |
| DLG2 | n.s. | n.s. | n.s. | −3.2 | n.s. |
| DLG5 | −2.1 | −1.9 | n.s. | n.s. | n.s. |
| DLG7 | 2.5 | −2.0 | n.s. | n.s. | n.s. |
| DLX1 | 2.3 | n.s. | n.s. | n.s. | n.s. |
| DLX2 | 17.1 | 2.5 | n.s. | n.s. | n.s. |
| DMD | −3.1 | n.s. | 2.0 | n.s. | n.s. |
| DMXL1 | −2.0 | n.s. | n.s. | n.s. | n.s. |
| DNAH2 | n.s. | n.s. | n.s. | 2.1 | n.s. |
| DNAJA4 | 2.1 | n.s. | n.s. | n.s. | n.s. |
| DNAJA5 | −2.2 | n.s. | n.s. | n.s. | n.s. |
| DNAJB1 | 2.4 | n.s. | n.s. | n.s. | n.s. |
| DNAJB14 | −1.8 | n.s. | n.s. | n.s. | n.s. |
| DNAJB2 | n.s. | n.s. | −2.3 | n.s. | n.s. |
| DNAJB5 | 3.3 | n.s. | n.s. | n.s. | n.s. |
| DNAJB9 | 3.2 | 2.1 | n.s. | n.s. | n.s. |
| DNAJC18 | −2.4 | −1.8 | n.s. | n.s. | n.s. |
| DNAJC3 | 3.2 | −5.1 | n.s. | n.s. | n.s. |
| DNAJC5 | 2.2 | n.s. | n.s. | n.s. | n.s. |
| DNAJC9 | 2.9 | n.s. | n.s. | n.s. | n.s. |
| DNAL1 | n.s. | −1.8 | n.s. | n.s. | n.s. |
| DNALI1 | −3.7 | −10.3 | n.s. | n.s. | n.s. |
| DNASE2 | n.s. | −1.9 | n.s. | n.s. | n.s. |
| DNM1 | −4.1 | n.s. | n.s. | n.s. | n.s. |
| DNM1L | −3.8 | n.s. | n.s. | n.s. | n.s. |
| DNM3 | −1.9 | −2.0 | n.s. | n.s. | n.s. |
| DNPEP | n.s. | −1.9 | n.s. | n.s. | n.s. |
| DOCK1 | −2.5 | 1.9 | n.s. | n.s. | n.s. |
| DOCK11 | −2.3 | n.s. | n.s. | n.s. | n.s. |
| DOCK3 | n.s. | n.s. | n.s. | n.s. | 2.3 |
| DOCK4 | −2.4 | n.s. | n.s. | n.s. | n.s. |
| DOCK5 | −2.2 | n.s. | n.s. | n.s. | n.s. |
| DOHH | 2.4 | n.s. | n.s. | n.s. | n.s. |
| DOT1L | 3.1 | 2.4 | n.s. | n.s. | n.s. |
| DPF3 | −2.2 | n.s. | n.s. | n.s. | n.s. |
| DPH2 | 1.9 | n.s. | n.s. | n.s. | n.s. |
| DPH3 | 1.9 | n.s. | n.s. | n.s. | n.s. |
| DPH5 | −1.8 | n.s. | n.s. | n.s. | n.s. |
| DPM2 | 2.2 | n.s. | n.s. | n.s. | n.s. |
| DPP4 | −3.5 | n.s. | n.s. | n.s. | n.s. |
| DPP8 | −2.4 | n.s. | n.s. | n.s. | n.s. |
| DPT | −3.4 | −1.9 | n.s. | n.s. | n.s. |
| DPY19L1 | −1.8 | n.s. | n.s. | n.s. | n.s. |
| DPY19L1P1 | n.s. | n.s. | 1.9 | n.s. | n.s. |
| DPY19L4 | −2.1 | n.s. | n.s. | n.s. | n.s. |
| DPYD | n.s. | 1.9 | n.s. | n.s. | n.s. |
| DPYSL2 | −2.4 | n.s. | n.s. | n.s. | n.s. |
| DPYSL3 | −2.3 | n.s. | n.s. | n.s. | n.s. |
| DSEL | −2.1 | −1.8 | n.s. | n.s. | n.s. |
| DSN1 | 2.2 | n.s. | n.s. | n.s. | n.s. |
| DTL | 4.8 | 2.4 | n.s. | n.s. | n.s. |
| DTNA | 2.7 | n.s. | n.s. | n.s. | n.s. |
| DTWD1 | −2.2 | n.s. | n.s. | n.s. | n.s. |
| DTWD2 | −4.4 | n.s. | 2.4 | n.s. | n.s. |
| DTX3L | −3.2 | n.s. | n.s. | n.s. | n.s. |
| DTYMK | 2.8 | n.s. | n.s. | n.s. | n.s. |
| DUSP1 | 4.3 | 3.4 | n.s. | n.s. | n.s. |
| DUSP10 | 2.5 | n.s. | n.s. | n.s. | n.s. |
| DUSP13 | n.s. | n.s. | n.s. | 3.8 | n.s. |
| DUSP14 | n.s. | 2.0 | n.s. | n.s. | n.s. |
| DUSP16 | 4.1 | n.s. | n.s. | n.s. | n.s. |
| DUSP18 | n.s. | −1.8 | n.s. | n.s. | n.s. |
| DUSP2 | 22.8 | 9.5 | n.s. | n.s. | n.s. |
| DUSP23 | 2.2 | n.s. | n.s. | n.s. | n.s. |
| DUSP3 | 3.2 | 2.3 | n.s. | n.s. | n.s. |
| DUSP4 | 3.2 | 3.9 | n.s. | n.s. | n.s. |
| DUSP5 | 3.0 | 3.5 | n.s. | n.s. | n.s. |
| DUSP6 | n.s. | 11.2 | n.s. | n.s. | n.s. |
| DUSP8 | 3.7 | n.s. | n.s. | n.s. | n.s. |
| DYM | −2.3 | n.s. | n.s. | n.s. | n.s. |
| DYNC1H1 | n.s. | 2.4 | n.s. | n.s. | n.s. |
| DYNC2H1 | −3.3 | n.s. | n.s. | n.s. | n.s. |
| DYNC2LI1 | −1.8 | n.s. | n.s. | n.s. | n.s. |
| DYNLL2 | 1.9 | n.s. | n.s. | n.s. | n.s. |
| DYRK1A | n.s. | n.s. | −1.8 | n.s. | n.s. |
| DYRK3 | 3.2 | n.s. | n.s. | n.s. | n.s. |
| DYSF | n.s. | n.s. | −2.4 | n.s. | n.s. |
| DZIP1 | −2.4 | n.s. | n.s. | n.s. | n.s. |
| DZIP3 | −2.7 | −3.3 | n.s. | n.s. | n.s. |
| E2F1 | 12.5 | 2.4 | n.s. | n.s. | n.s. |
| E2F7 | 6.1 | 2.4 | n.s. | n.s. | n.s. |
| E2F8 | n.s. | n.s. | n.s. | 1.9 | 1.9 |
| E4F1 | n.s. | n.s. | −3.1 | n.s. | n.s. |
| EBF | −3.8 | −3.7 | n.s. | n.s. | n.s. |
| EBF1 | −3.6 | −3.7 | n.s. | n.s. | n.s. |
| EBF2 | −2.0 | n.s. | n.s. | n.s. | n.s. |
| EBI2 | n.s. | 2.8 | n.s. | n.s. | n.s. |
| ECE2 | 3.4 | n.s. | n.s. | n.s. | n.s. |
| ECH1 | −2.3 | n.s. | n.s. | n.s. | n.s. |
| ECHDC2 | −2.2 | n.s. | n.s. | n.s. | n.s. |
| ECM2 | −2.7 | n.s. | n.s. | n.s. | n.s. |
| EDC3 | 1.8 | n.s. | n.s. | n.s. | n.s. |
| EDEM1 | n.s. | −2.5 | n.s. | n.s. | n.s. |
| EDG1 | 1.9 | 2.0 | n.s. | n.s. | n.s. |
| EDG2 | 2.0 | −2.3 | n.s. | n.s. | n.s. |
| EDG5 | −2.6 | n.s. | n.s. | n.s. | n.s. |
| EDNRA | 3.9 | 7.9 | n.s. | n.s. | n.s. |
| EEA1 | −2.7 | n.s. | n.s. | n.s. | n.s. |
| EEF2K | −1.9 | n.s. | n.s. | n.s. | n.s. |
| EFCAB2 | n.s. | −2.0 | n.s. | n.s. | n.s. |
| EFCAB4B | −2.2 | n.s. | n.s. | n.s. | n.s. |
| EFCAB6 | −3.8 | n.s. | n.s. | n.s. | n.s. |
| EFHA2 | −2.6 | n.s. | n.s. | n.s. | n.s. |
| EFHC1 | −2.7 | −2.3 | n.s. | n.s. | n.s. |
| EFHD2 | 2.7 | 1.8 | n.s. | n.s. | n.s. |
| EFNA4 | n.s. | −1.9 | n.s. | n.s. | n.s. |
| EFNB2 | n.s. | 2.9 | n.s. | n.s. | n.s. |
| EFNB3 | −13.0 | n.s. | n.s. | n.s. | n.s. |
| EGFR | n.s. | 2.6 | n.s. | n.s. | n.s. |
| EGR1 | 3.1 | 2.5 | n.s. | n.s. | n.s. |
| EGR2 | −6.6 | n.s. | 2.5 | n.s. | n.s. |
| EGR3 | n.s. | 1.9 | n.s. | n.s. | n.s. |
| EHBP1 | −3.1 | n.s. | n.s. | n.s. | n.s. |
| EHD1 | n.s. | 2.8 | n.s. | n.s. | n.s. |
| EHD3 | −2.5 | n.s. | n.s. | n.s. | n.s. |
| EHD4 | n.s. | 2.0 | n.s. | n.s. | n.s. |
| EHF | n.s. | n.s. | n.s. | n.s. | 4.8 |
| EID3 | −1.8 | n.s. | n.s. | n.s. | n.s. |
| EIF1 | 2.7 | n.s. | n.s. | n.s. | n.s. |
| EIF2C1 | n.s. | −1.9 | n.s. | n.s. | n.s. |
| EIF2C4 | −2.0 | n.s. | n.s. | n.s. | n.s. |
| EIF2S1 | 2.0 | n.s. | n.s. | n.s. | n.s. |
| EIF2S2 | −2.0 | n.s. | n.s. | n.s. | n.s. |
| EIF2S3 | −2.3 | n.s. | n.s. | n.s. | n.s. |
| EIF3A | −2.6 | n.s. | n.s. | n.s. | n.s. |
| EIF3S9 | n.s. | 1.9 | n.s. | n.s. | n.s. |
| EIF4A2 | −2.9 | n.s. | n.s. | n.s. | n.s. |
| EIF4A3 | 2.0 | n.s. | n.s. | n.s. | n.s. |
| EIF4B | n.s. | n.s. | −1.8 | n.s. | n.s. |
| EIF4E | 4.7 | 4.5 | n.s. | n.s. | n.s. |
| EIF4E3 | −2.2 | n.s. | n.s. | n.s. | n.s. |
| EIF4EBP2 | n.s. | 2.1 | n.s. | n.s. | n.s. |
| EIF4G1 | n.s. | n.s. | −1.8 | n.s. | n.s. |
| EIF5A2 | −1.9 | n.s. | n.s. | n.s. | n.s. |
| ELK4 | −2.2 | n.s. | n.s. | n.s. | n.s. |
| ELL2 | 3.4 | 4.5 | n.s. | n.s. | n.s. |
| ELL3 | n.s. | −2.4 | n.s. | n.s. | n.s. |
| ELMO2 | n.s. | n.s. | n.s. | −2.6 | n.s. |
| ELMOD1 | n.s. | 4.8 | n.s. | n.s. | n.s. |
| ELMOD2 | 1.9 | n.s. | n.s. | n.s. | n.s. |
| ELOVL6 | n.s. | 2.3 | n.s. | n.s. | n.s. |
| EMG1 | 2.0 | n.s. | n.s. | n.s. | n.s. |
| EML2 | n.s. | 2.0 | n.s. | n.s. | n.s. |
| EML4 | −1.8 | n.s. | n.s. | n.s. | n.s. |
| EMP1 | n.s. | 3.0 | n.s. | −2.0 | n.s. |
| EMX2 | −2.5 | −1.9 | n.s. | n.s. | n.s. |
| EMX2OS | −2.7 | −1.9 | n.s. | n.s. | n.s. |
| ENAH | 1.8 | n.s. | n.s. | n.s. | n.s. |
| ENC1 | 1.8 | 2.4 | n.s. | n.s. | n.s. |
| ENDOD1 | 2.2 | 1.9 | n.s. | n.s. | n.s. |
| ENDOG | 2.0 | n.s. | n.s. | n.s. | n.s. |
| ENG | n.s. | 1.8 | n.s. | n.s. | n.s. |
| ENO1B | n.s. | n.s. | −2.2 | n.s. | n.s. |
| ENO2 | 3.0 | 2.1 | −1.9 | n.s. | n.s. |
| ENOX1 | −4.1 | −2.3 | n.s. | n.s. | n.s. |
| ENPP2 | −2.0 | n.s. | n.s. | n.s. | n.s. |
| ENST00000256861 | n.s. | n.s. | 1.9 | n.s. | n.s. |
| ENST00000302942 | n.s. | n.s. | n.s. | 21.1 | n.s. |
| ENST00000306515 | n.s. | n.s. | n.s. | 2.6 | n.s. |
| ENST00000327781 | n.s. | n.s. | n.s. | −3.2 | n.s. |
| ENST00000342829 | n.s. | n.s. | n.s. | −2.2 | n.s. |
| ENST00000354343 | n.s. | n.s. | n.s. | n.s. | 2.0 |
| ENST00000356104 | n.s. | n.s. | n.s. | 4.5 | 3.0 |
| ENST00000366930 | n.s. | n.s. | 2.0 | n.s. | n.s. |
| ENST00000366971 | n.s. | n.s. | n.s. | n.s. | 1.9 |
| ENST00000371408 | n.s. | n.s. | n.s. | n.s. | 2.1 |
| ENST00000379108 | n.s. | n.s. | n.s. | 2.4 | n.s. |
| ENST00000379131 | n.s. | n.s. | n.s. | 1.8 | n.s. |
| ENST00000380357 | n.s. | n.s. | n.s. | 2.1 | n.s. |
| ENTPD7 | 4.7 | n.s. | n.s. | n.s. | n.s. |
| EP300 | n.s. | n.s. | n.s. | −2.1 | −2.4 |
| EPAS1 | n.s. | 4.0 | n.s. | n.s. | n.s. |
| EPB41 | n.s. | −2.0 | n.s. | n.s. | n.s. |
| EPB41L1 | −2.1 | n.s. | n.s. | n.s. | n.s. |
| EPB41L2 | −2.8 | n.s. | n.s. | n.s. | n.s. |
| EPB41L3 | −2.3 | n.s. | n.s. | n.s. | n.s. |
| EPB41L4B | n.s. | n.s. | 2.5 | n.s. | n.s. |
| EPB41L5 | −2.0 | n.s. | n.s. | n.s. | n.s. |
| EPHA2 | n.s. | 5.1 | n.s. | n.s. | n.s. |
| EPHA4 | n.s. | −4.3 | n.s. | n.s. | n.s. |
| EPHB1 | 4.6 | 10.4 | n.s. | n.s. | n.s. |
| EPHB4 | −2.1 | n.s. | n.s. | n.s. | n.s. |
| EPOR | n.s. | −2.1 | n.s. | n.s. | n.s. |
| EPPB9 | −1.8 | n.s. | n.s. | n.s. | n.s. |
| EPR1 | 2.7 | n.s. | n.s. | 2.2 | n.s. |
| EPRS | −2.3 | −2.2 | n.s. | n.s. | n.s. |
| EPSTI1 | −2.8 | n.s. | n.s. | n.s. | n.s. |
| ERBB2IP | −1.9 | n.s. | n.s. | n.s. | n.s. |
| ERBB3 | n.s. | −2.3 | n.s. | n.s. | n.s. |
| ERCC1 | n.s. | 1.9 | n.s. | n.s. | n.s. |
| ERCC2 | n.s. | 2.0 | n.s. | n.s. | n.s. |
| ERCC4 | −2.0 | n.s. | n.s. | n.s. | n.s. |
| ERF | 1.9 | n.s. | n.s. | n.s. | n.s. |
| ERGIC2 | −2.1 | n.s. | n.s. | n.s. | n.s. |
| ERMAP | −2.7 | −1.9 | n.s. | n.s. | n.s. |
| ERMP1 | −2.0 | n.s. | n.s. | n.s. | n.s. |
| ERN1 | 2.8 | n.s. | n.s. | n.s. | n.s. |
| ERRFI1 | 1.8 | 2.2 | n.s. | n.s. | n.s. |
| ESCO2 | 4.5 | n.s. | n.s. | n.s. | n.s. |
| ESM1 | 3.6 | 4.1 | n.s. | n.s. | n.s. |
| ESPL1 | 2.5 | n.s. | n.s. | n.s. | n.s. |
| ETAA1 | −2.5 | n.s. | n.s. | n.s. | n.s. |
| ETNK1 | 3.7 | n.s. | n.s. | n.s. | n.s. |
| ETS1 | 3.5 | 2.5 | n.s. | n.s. | n.s. |
| ETS2 | 2.9 | n.s. | n.s. | n.s. | n.s. |
| ETV1 | −2.3 | n.s. | n.s. | n.s. | n.s. |
| ETV4 | n.s. | n.s. | −2.1 | n.s. | −2.0 |
| ETV5 | n.s. | 3.1 | n.s. | n.s. | n.s. |
| EVC | −4.1 | n.s. | n.s. | n.s. | n.s. |
| EVC2 | −2.0 | n.s. | n.s. | n.s. | n.s. |
| EVI1 | 6.4 | n.s. | n.s. | n.s. | n.s. |
| EVI2A | n.s. | −2.8 | n.s. | n.s. | n.s. |
| EVI2B | n.s. | −3.1 | n.s. | n.s. | n.s. |
| EVI5 | −2.2 | −1.8 | n.s. | n.s. | n.s. |
| EVL | n.s. | n.s. | n.s. | n.s. | −1.8 |
| EWSR1 | n.s. | n.s. | −1.8 | n.s. | n.s. |
| EXO1 | 3.3 | 2.4 | n.s. | n.s. | n.s. |
| EXOC3 | −2.6 | −2.1 | n.s. | n.s. | n.s. |
| EXOC4 | −1.9 | n.s. | n.s. | n.s. | n.s. |
| EXOSC2 | 1.9 | n.s. | n.s. | n.s. | n.s. |
| EXOSC4 | 3.0 | 1.9 | n.s. | n.s. | n.s. |
| EXOSC5 | 2.0 | n.s. | n.s. | n.s. | n.s. |
| EXOSC6 | −1.9 | −1.9 | n.s. | n.s. | n.s. |
| EXT1 | 3.3 | 5.8 | n.s. | n.s. | n.s. |
| EYA2 | n.s. | 3.1 | n.s. | n.s. | n.s. |
| EZH1 | −5.0 | n.s. | n.s. | n.s. | n.s. |
| EZH2 | 2.2 | n.s. | n.s. | n.s. | n.s. |
| F10 | −1.9 | n.s. | n.s. | n.s. | n.s. |
| F2RL1 | 4.4 | 8.0 | n.s. | n.s. | n.s. |
| F2RL3 | n.s. | n.s. | −2.2 | n.s. | n.s. |
| F3 | n.s. | 8.6 | n.s. | n.s. | n.s. |
| FABP5 | 3.1 | n.s. | n.s. | n.s. | n.s. |
| FABP6 | n.s. | n.s. | n.s. | n.s. | −1.9 |
| FAM100A | 2.0 | n.s. | n.s. | n.s. | n.s. |
| FAM100B | 2.5 | n.s. | n.s. | n.s. | n.s. |
| FAM102B | −3.7 | −2.1 | n.s. | n.s. | n.s. |
| FAM105B | 1.9 | n.s. | n.s. | n.s. | n.s. |
| FAM108C1 | −2.6 | n.s. | n.s. | n.s. | n.s. |
| FAM110B | −2.2 | −2.0 | n.s. | n.s. | n.s. |
| FAM112A | 3.7 | n.s. | n.s. | n.s. | n.s. |
| FAM113A | n.s. | n.s. | −1.8 | n.s. | n.s. |
| FAM114A1 | −2.1 | n.s. | n.s. | n.s. | n.s. |
| FAM115A | −2.2 | −1.8 | n.s. | n.s. | n.s. |
| FAM120B | −1.9 | n.s. | n.s. | n.s. | n.s. |
| FAM122A | n.s. | −2.1 | n.s. | n.s. | n.s. |
| FAM122C | n.s. | n.s. | 4.5 | n.s. | n.s. |
| FAM126A | −2.1 | n.s. | n.s. | n.s. | n.s. |
| FAM126B | −2.0 | −2.4 | n.s. | n.s. | n.s. |
| FAM129A | −2.1 | −2.5 | n.s. | n.s. | n.s. |
| FAM130A1 | 2.8 | n.s. | n.s. | n.s. | n.s. |
| FAM133A | n.s. | n.s. | n.s. | 2.4 | n.s. |
| FAM134B | n.s. | −3.1 | n.s. | n.s. | n.s. |
| FAM135A | −2.2 | n.s. | n.s. | n.s. | n.s. |
| FAM13A1 | −2.1 | −2.3 | n.s. | n.s. | n.s. |
| FAM13C1 | −3.3 | n.s. | n.s. | n.s. | n.s. |
| FAM20A | −2.0 | n.s. | n.s. | n.s. | n.s. |
| FAM21C | −1.8 | n.s. | n.s. | n.s. | n.s. |
| FAM27A | 3.2 | n.s. | n.s. | n.s. | n.s. |
| FAM29A | 1.9 | n.s. | n.s. | n.s. | n.s. |
| FAM3A | n.s. | n.s. | n.s. | n.s. | −1.8 |
| FAM40B | n.s. | n.s. | n.s. | n.s. | 4.2 |
| FAM44A | n.s. | −2.7 | n.s. | n.s. | n.s. |
| FAM46A | −1.8 | −2.5 | n.s. | n.s. | n.s. |
| FAM54A | 5.9 | n.s. | n.s. | n.s. | n.s. |
| FAM55C | −2.3 | −2.2 | n.s. | n.s. | n.s. |
| FAM58A | 1.8 | n.s. | n.s. | n.s. | n.s. |
| FAM60A | 2.1 | 2.2 | n.s. | n.s. | n.s. |
| FAM62B | −1.8 | n.s. | n.s. | n.s. | n.s. |
| FAM63A | −2.0 | n.s. | −1.9 | n.s. | n.s. |
| FAM63B | −2.3 | −2.3 | n.s. | n.s. | n.s. |
| FAM64A | 3.5 | −2.2 | n.s. | n.s. | n.s. |
| FAM65A | 2.3 | 2.4 | n.s. | n.s. | n.s. |
| FAM72A | 3.8 | n.s. | n.s. | n.s. | n.s. |
| FAM73A | −1.8 | −1.9 | n.s. | n.s. | n.s. |
| FAM73B | n.s. | n.s. | n.s. | n.s. | −1.9 |
| FAM76A | −1.8 | n.s. | n.s. | n.s. | n.s. |
| FAM83D | 3.6 | n.s. | n.s. | n.s. | n.s. |
| FAM83G | 3.5 | n.s. | n.s. | n.s. | n.s. |
| FAM83H | n.s. | n.s. | 4.9 | n.s. | n.s. |
| FAM84A | −2.6 | n.s. | n.s. | n.s. | n.s. |
| FAM84B | n.s. | n.s. | n.s. | n.s. | 3.4 |
| FAM87A | 6.3 | n.s. | n.s. | n.s. | n.s. |
| FAM8A1 | −2.7 | −2.5 | n.s. | n.s. | n.s. |
| FAM92A1 | −2.2 | n.s. | n.s. | n.s. | n.s. |
| FANCA | 3.2 | 2.2 | n.s. | n.s. | n.s. |
| FANCF | n.s. | −1.8 | n.s. | n.s. | n.s. |
| FANCG | 2.1 | n.s. | n.s. | n.s. | n.s. |
| FANCI | 3.3 | n.s. | n.s. | n.s. | n.s. |
| FAP | −2.5 | n.s. | n.s. | n.s. | n.s. |
| FARP1 | −1.9 | 1.9 | n.s. | n.s. | n.s. |
| FARP2 | −2.1 | −2.2 | n.s. | n.s. | n.s. |
| FARSA | 2.3 | n.s. | n.s. | n.s. | n.s. |
| FASN | n.s. | 2.6 | n.s. | n.s. | n.s. |
| FASTKD5 | 2.9 | n.s. | n.s. | n.s. | n.s. |
| FAT4 | −2.4 | n.s. | n.s. | n.s. | n.s. |
| FBLN1 | −2.4 | n.s. | n.s. | n.s. | n.s. |
| FBLN2 | −1.9 | n.s. | n.s. | n.s. | n.s. |
| FBLN7 | −2.5 | n.s. | n.s. | n.s. | n.s. |
| FBN2 | −2.3 | n.s. | n.s. | n.s. | n.s. |
| FBXL14 | −1.8 | n.s. | n.s. | n.s. | n.s. |
| FBXL17 | −2.7 | n.s. | n.s. | n.s. | n.s. |
| FBXL20 | n.s. | −2.0 | n.s. | n.s. | n.s. |
| FBXL4 | −2.1 | −1.9 | n.s. | n.s. | n.s. |
| FBXL6 | 2.5 | 2.1 | n.s. | n.s. | n.s. |
| FBXL7 | −2.8 | n.s. | n.s. | n.s. | n.s. |
| FBXO16 | −3.4 | −2.3 | n.s. | n.s. | n.s. |
| FBXO30 | 2.0 | n.s. | n.s. | n.s. | n.s. |
| FBXO31 | n.s. | −1.8 | n.s. | n.s. | n.s. |
| FBXO32 | 14.7 | n.s. | n.s. | n.s. | n.s. |
| FBXO33 | n.s. | −1.8 | n.s. | n.s. | n.s. |
| FBXO45 | 1.9 | n.s. | n.s. | n.s. | n.s. |
| FBXO9 | −3.6 | n.s. | n.s. | n.s. | n.s. |
| FBXW2 | −2.2 | n.s. | n.s. | n.s. | n.s. |
| FBXW7 | n.s. | −1.9 | n.s. | n.s. | n.s. |
| FCMD | −2.4 | n.s. | n.s. | n.s. | n.s. |
| FDPSL2A | n.s. | n.s. | −1.8 | n.s. | n.s. |
| FDXR | n.s. | −1.9 | n.s. | n.s. | n.s. |
| FECH | −1.9 | n.s. | n.s. | n.s. | n.s. |
| FEN1 | 3.6 | 2.1 | n.s. | n.s. | n.s. |
| FER1L3 | n.s. | n.s. | n.s. | −1.8 | −2.0 |
| FGF1 | n.s. | 3.7 | n.s. | n.s. | n.s. |
| FGF18 | 12.3 | n.s. | n.s. | n.s. | n.s. |
| FGF5 | −2.4 | 2.5 | n.s. | n.s. | n.s. |
| FGFR1OP | −2.5 | −2.0 | n.s. | n.s. | n.s. |
| FGFR3 | 3.7 | n.s. | n.s. | n.s. | n.s. |
| FGFRL1 | 2.5 | n.s. | n.s. | n.s. | n.s. |
| FHL1 | −2.3 | n.s. | n.s. | n.s. | n.s. |
| FHOD1 | 2.8 | 2.3 | n.s. | n.s. | n.s. |
| FIBCD1 | 3.4 | n.s. | n.s. | n.s. | n.s. |
| FIBIN | −3.4 | −3.3 | n.s. | n.s. | n.s. |
| FIG4 | −1.9 | n.s. | n.s. | n.s. | n.s. |
| FIGNL1 | 1.9 | n.s. | n.s. | n.s. | n.s. |
| FILIP1L | −1.8 | n.s. | n.s. | n.s. | n.s. |
| FIP1L1 | n.s. | n.s. | n.s. | n.s. | −1.9 |
| FIS1 | n.s. | −2.5 | n.s. | n.s. | n.s. |
| FJX1 | n.s. | 3.6 | n.s. | n.s. | n.s. |
| FKBP1A | 2.9 | n.s. | n.s. | n.s. | n.s. |
| FKBP4 | 2.4 | n.s. | −2.1 | n.s. | n.s. |
| FKBP5 | 2.4 | 2.0 | n.s. | n.s. | n.s. |
| FKBPL | n.s. | n.s. | −1.9 | n.s. | n.s. |
| FKSG12 | −4.3 | −2.1 | n.s. | n.s. | n.s. |
| FKSG24 | 2.6 | n.s. | n.s. | n.s. | n.s. |
| FLJ10292 | 1.9 | n.s. | n.s. | n.s. | n.s. |
| FLJ10357 | −2.0 | n.s. | n.s. | n.s. | n.s. |
| FLJ10769 | n.s. | n.s. | n.s. | −1.8 | n.s. |
| FLJ10815 | 1.8 | n.s. | n.s. | n.s. | n.s. |
| FLJ10986 | −3.8 | n.s. | n.s. | n.s. | n.s. |
| FLJ10996 | n.s. | 2.8 | n.s. | n.s. | n.s. |
| FLJ11000 | −2.0 | n.s. | n.s. | n.s. | n.s. |
| FLJ11151 | 2.1 | 2.0 | n.s. | n.s. | n.s. |
| FLJ11286 | −1.9 | n.s. | n.s. | n.s. | n.s. |
| FLJ11736 | n.s. | n.s. | 2.0 | n.s. | n.s. |
| FLJ11806 | −2.2 | n.s. | n.s. | n.s. | n.s. |
| FLJ11996 | n.s. | n.s. | 2.6 | n.s. | n.s. |
| FLJ13231 | −3.4 | n.s. | n.s. | n.s. | n.s. |
| FLJ14213 | 4.1 | 2.8 | n.s. | n.s. | n.s. |
| FLJ20030 | −1.9 | n.s. | n.s. | n.s. | n.s. |
| FLJ20035 | −2.6 | n.s. | n.s. | n.s. | n.s. |
| FLJ20309 | n.s. | −2.6 | n.s. | n.s. | n.s. |
| FLJ20433 | −2.0 | n.s. | n.s. | n.s. | n.s. |
| FLJ21616 | n.s. | −1.9 | n.s. | n.s. | n.s. |
| FLJ21777 | n.s. | n.s. | n.s. | 2.3 | n.s. |
| FLJ21986 | −2.1 | n.s. | n.s. | n.s. | n.s. |
| FLJ22639 | n.s. | −2.2 | n.s. | n.s. | n.s. |
| FLJ22659 | n.s. | n.s. | n.s. | n.s. | −2.4 |
| FLJ22662 | n.s. | n.s. | n.s. | 3.2 | n.s. |
| FLJ23556 | n.s. | 2.3 | n.s. | n.s. | n.s. |
| FLJ23754 | n.s. | n.s. | n.s. | −1.8 | n.s. |
| FLJ23861 | n.s. | −2.4 | n.s. | n.s. | n.s. |
| FLJ23867 | n.s. | n.s. | −1.8 | n.s. | n.s. |
| FLJ25006 | −2.4 | −2.3 | n.s. | n.s. | n.s. |
| FLJ25328 | n.s. | n.s. | n.s. | −3.2 | n.s. |
| FLJ25778 | n.s. | −2.3 | n.s. | n.s. | n.s. |
| FLJ27365 | 1.9 | n.s. | n.s. | n.s. | n.s. |
| FLJ30851 | −2.1 | n.s. | n.s. | n.s. | n.s. |
| FLJ31306 | −3.0 | n.s. | n.s. | n.s. | n.s. |
| FLJ31401 | n.s. | n.s. | n.s. | −2.4 | n.s. |
| FLJ32679 | −2.1 | −2.6 | n.s. | n.s. | n.s. |
| FLJ33674 | 1.9 | n.s. | n.s. | n.s. | n.s. |
| FLJ33996 | −3.0 | n.s. | n.s. | n.s. | n.s. |
| FLJ34208 | n.s. | 2.7 | n.s. | n.s. | n.s. |
| FLJ35348 | n.s. | −2.9 | n.s. | n.s. | n.s. |
| FLJ35379 | n.s. | n.s. | −2.6 | −5.8 | −3.2 |
| FLJ36701 | 7.3 | n.s. | n.s. | n.s. | n.s. |
| FLJ38348 | −2.7 | −1.9 | n.s. | n.s. | n.s. |
| FLJ38717 | n.s. | 3.5 | n.s. | n.s. | n.s. |
| FLJ38973 | n.s. | n.s. | n.s. | 2.0 | 2.0 |
| FLJ39051 | −2.0 | n.s. | n.s. | n.s. | n.s. |
| FLJ39653 | n.s. | n.s. | n.s. | 3.7 | n.s. |
| FLJ39660 | 2.3 | n.s. | n.s. | n.s. | n.s. |
| FLJ40113 | n.s. | n.s. | −2.0 | n.s. | n.s. |
| FLJ40142 | −1.9 | n.s. | n.s. | n.s. | n.s. |
| FLJ40330 | n.s. | n.s. | n.s. | 3.3 | n.s. |
| FLJ42393 | n.s. | 1.9 | n.s. | n.s. | n.s. |
| FLJ42709 | 2.2 | 2.1 | n.s. | n.s. | n.s. |
| FLJ43276 | −2.6 | −2.6 | n.s. | n.s. | n.s. |
| FLJ43663 | 3.3 | −2.4 | n.s. | n.s. | n.s. |
| FLJ43692 | n.s. | n.s. | n.s. | n.s. | −1.8 |
| FLJ44342 | 1.9 | n.s. | n.s. | n.s. | n.s. |
| FLJ44635 | −11.0 | −2.6 | n.s. | n.s. | n.s. |
| FLJ90757 | −2.7 | −3.4 | n.s. | n.s. | n.s. |
| FLNA | n.s. | 2.1 | n.s. | n.s. | n.s. |
| FLNB | n.s. | 2.6 | n.s. | n.s. | n.s. |
| FLNC | 2.8 | 3.4 | n.s. | n.s. | n.s. |
| FLOT1 | −2.6 | −3.1 | −2.2 | n.s. | n.s. |
| FLRT3 | n.s. | 2.6 | n.s. | n.s. | n.s. |
| FLT3LG | −2.3 | n.s. | n.s. | n.s. | n.s. |
| FLYWCH2 | n.s. | −1.8 | n.s. | n.s. | n.s. |
| FMNL1 | n.s. | n.s. | n.s. | −2.5 | n.s. |
| FMNL2 | −2.2 | n.s. | n.s. | n.s. | n.s. |
| FMNL3 | −1.9 | n.s. | n.s. | n.s. | n.s. |
| FN1 | −2.4 | n.s. | n.s. | n.s. | n.s. |
| FNBP1 | −2.6 | −1.9 | n.s. | n.s. | n.s. |
| FNDC3B | 2.2 | 2.1 | n.s. | n.s. | n.s. |
| FNIP1 | 10.4 | 2.0 | n.s. | n.s. | n.s. |
| FOLR3 | 13.4 | 2.4 | n.s. | n.s. | n.s. |
| FOS | 48.7 | 54.8 | n.s. | n.s. | n.s. |
| FOSB | 67.3 | 51.4 | n.s. | n.s. | n.s. |
| FOSL1 | 2.2 | 2.7 | n.s. | n.s. | n.s. |
| FOSL2 | 1.8 | n.s. | n.s. | n.s. | n.s. |
| FOXC1 | −2.0 | n.s. | n.s. | n.s. | n.s. |
| FOXC2 | n.s. | 3.6 | n.s. | n.s. | n.s. |
| FOXL2 | n.s. | −1.9 | n.s. | n.s. | n.s. |
| FOXO1 | n.s. | −2.1 | n.s. | n.s. | n.s. |
| FOXO3A | n.s. | 2.2 | n.s. | n.s. | n.s. |
| FOXO4 | n.s. | n.s. | −1.8 | n.s. | −1.9 |
| FOXP1 | n.s. | 3.3 | n.s. | n.s. | n.s. |
| FOXQ1 | 5.1 | 2.3 | n.s. | n.s. | n.s. |
| FOXRED2 | n.s. | −3.0 | n.s. | n.s. | n.s. |
| FPGT | −3.1 | −2.0 | n.s. | n.s. | n.s. |
| FREQ | 4.1 | 2.2 | n.s. | n.s. | n.s. |
| FRMD3 | 3.8 | 4.8 | n.s. | n.s. | n.s. |
| FRMD4A | n.s. | 2.6 | n.s. | n.s. | n.s. |
| FRMPD4 | −6.1 | n.s. | n.s. | n.s. | n.s. |
| FRY | −4.4 | n.s. | n.s. | n.s. | n.s. |
| FSCN1 | n.s. | 1.9 | n.s. | n.s. | n.s. |
| FSHPRH1 | 2.0 | n.s. | n.s. | n.s. | n.s. |
| FST | −2.1 | 1.9 | n.s. | n.s. | n.s. |
| FSTL3 | 6.4 | 3.2 | n.s. | n.s. | n.s. |
| FTHL16 | −1.8 | −1.9 | n.s. | n.s. | n.s. |
| FUBP1 | −1.8 | −1.9 | n.s. | n.s. | n.s. |
| FURIN | 2.2 | 2.1 | n.s. | n.s. | n.s. |
| FUS | 2.5 | 2.4 | −2.0 | n.s. | n.s. |
| FUT1 | −2.5 | n.s. | n.s. | n.s. | n.s. |
| FUT4 | −2.2 | n.s. | n.s. | n.s. | n.s. |
| FVT1 | n.s. | 2.0 | n.s. | n.s. | n.s. |
| FXR1 | −1.8 | n.s. | n.s. | n.s. | n.s. |
| FXR2 | 2.5 | n.s. | n.s. | n.s. | n.s. |
| FYCO1 | −2.0 | n.s. | n.s. | n.s. | n.s. |
| FYN | −1.8 | n.s. | n.s. | n.s. | n.s. |
| FZD5 | 2.9 | n.s. | n.s. | n.s. | n.s. |
| FZD8 | 4.9 | 5.0 | n.s. | n.s. | n.s. |
| G0S2 | 3.8 | 2.5 | n.s. | n.s. | n.s. |
| G3BP1 | n.s. | 2.0 | n.s. | n.s. | n.s. |
| G6PC3 | −2.1 | n.s. | n.s. | n.s. | n.s. |
| GAB1 | −2.1 | −1.9 | n.s. | n.s. | n.s. |
| GABBR1 | −2.3 | n.s. | n.s. | n.s. | n.s. |
| GABBR2 | 5.4 | 3.2 | n.s. | n.s. | n.s. |
| GABPA | −2.0 | n.s. | n.s. | n.s. | n.s. |
| GABPB2 | 2.9 | n.s. | n.s. | n.s. | n.s. |
| GABRA2 | n.s. | n.s. | 25.2 | n.s. | n.s. |
| GABRB3 | −2.9 | n.s. | n.s. | n.s. | n.s. |
| GABRE | −3.1 | −2.4 | n.s. | n.s. | n.s. |
| GADD45A | 3.3 | n.s. | n.s. | n.s. | n.s. |
| GADD45B | 7.3 | 4.3 | n.s. | n.s. | n.s. |
| GAFA1 | 3.4 | n.s. | n.s. | n.s. | n.s. |
| GAK | 1.9 | 2.1 | n.s. | n.s. | n.s. |
| GAL | 4.4 | n.s. | n.s. | n.s. | n.s. |
| GAL3ST4 | −3.2 | n.s. | −1.9 | n.s. | n.s. |
| GALE | 2.5 | 1.8 | n.s. | n.s. | n.s. |
| GALM | n.s. | −1.8 | n.s. | n.s. | n.s. |
| GALNT12 | −2.5 | n.s. | n.s. | n.s. | n.s. |
| GALNT4 | n.s. | n.s. | n.s. | 1.9 | n.s. |
| GALNT5 | −1.9 | −1.9 | n.s. | n.s. | n.s. |
| GALNTL2 | n.s. | 1.8 | n.s. | n.s. | n.s. |
| GALT | −2.8 | n.s. | n.s. | n.s. | n.s. |
| GANAB | −1.8 | n.s. | n.s. | n.s. | n.s. |
| GART | n.s. | 2.4 | n.s. | n.s. | n.s. |
| GAS1 | −5.4 | −2.6 | n.s. | n.s. | n.s. |
| GAS2L3 | n.s. | −3.9 | n.s. | 1.9 | n.s. |
| GAS7 | n.s. | 2.7 | n.s. | n.s. | n.s. |
| GATA2 | 3.1 | n.s. | n.s. | n.s. | n.s. |
| GATA6 | −2.5 | n.s. | 2.3 | n.s. | n.s. |
| GATAD1 | −1.9 | n.s. | n.s. | n.s. | n.s. |
| GATAD2B | −3.8 | −2.4 | n.s. | n.s. | n.s. |
| GBA2 | n.s. | n.s. | −1.9 | −1.9 | n.s. |
| GBP2 | −2.5 | n.s. | n.s. | n.s. | n.s. |
| GBP3 | −1.9 | n.s. | n.s. | n.s. | n.s. |
| GCC2 | −1.9 | n.s. | n.s. | n.s. | n.s. |
| GCH1 | 2.4 | n.s. | n.s. | n.s. | n.s. |
| GCL | n.s. | −2.3 | n.s. | n.s. | n.s. |
| GCLC | 2.9 | n.s. | n.s. | n.s. | n.s. |
| GCLM | 2.7 | n.s. | n.s. | n.s. | n.s. |
| GCN1L1 | n.s. | 2.0 | n.s. | n.s. | n.s. |
| GCNT1 | n.s. | −2.0 | n.s. | n.s. | n.s. |
| Gcom1 | −1.9 | −2.2 | n.s. | n.s. | n.s. |
| GCS1 | n.s. | 1.9 | n.s. | n.s. | n.s. |
| GDF15 | 9.1 | 1.8 | n.s. | n.s. | n.s. |
| GDF5 | n.s. | −13.6 | n.s. | n.s. | n.s. |
| GDF6 | n.s. | n.s. | 2.5 | 2.6 | n.s. |
| GDPD1 | n.s. | −2.8 | n.s. | n.s. | n.s. |
| GEM | 10.3 | 2.9 | n.s. | n.s. | n.s. |
| GEMIN4 | 2.4 | 2.6 | n.s. | n.s. | n.s. |
| GEMIN8 | −1.8 | n.s. | n.s. | n.s. | n.s. |
| GFOD1 | 2.1 | n.s. | n.s. | n.s. | n.s. |
| GFPT2 | n.s. | 2.2 | n.s. | n.s. | n.s. |
| GGA2 | −2.9 | n.s. | n.s. | n.s. | n.s. |
| GGCX | 2.0 | n.s. | n.s. | n.s. | n.s. |
| GGT1 | n.s. | n.s. | n.s. | −1.9 | n.s. |
| GHR | −1.9 | n.s. | n.s. | n.s. | n.s. |
| GIMAP2 | −3.9 | n.s. | n.s. | n.s. | n.s. |
| GINS1 | 3.2 | 1.9 | n.s. | n.s. | n.s. |
| GINS2 | 4.5 | 2.0 | n.s. | n.s. | n.s. |
| GINS3 | 8.3 | n.s. | n.s. | n.s. | n.s. |
| GINS4 | 5.2 | n.s. | n.s. | n.s. | n.s. |
| GIPC2 | n.s. | n.s. | n.s. | −2.8 | n.s. |
| GIPR | n.s. | n.s. | 2.0 | 2.3 | n.s. |
| GIYD2 | 5.7 | n.s. | n.s. | n.s. | n.s. |
| GJA1 | −2.2 | n.s. | n.s. | n.s. | n.s. |
| GJA7 | −1.8 | n.s. | n.s. | n.s. | n.s. |
| GJC1 | −2.0 | n.s. | n.s. | n.s. | n.s. |
| GK | 6.2 | 2.7 | n.s. | n.s. | n.s. |
| GK3P | 3.6 | 1.9 | n.s. | n.s. | n.s. |
| GK5 | n.s. | −2.2 | n.s. | n.s. | n.s. |
| GKAP1 | −2.3 | −2.7 | n.s. | n.s. | n.s. |
| GLA | 2.3 | n.s. | n.s. | n.s. | n.s. |
| GLCCI1 | n.s. | −2.3 | n.s. | n.s. | n.s. |
| GLG1 | −1.8 | n.s. | n.s. | n.s. | n.s. |
| GLI3 | −3.9 | −2.2 | n.s. | n.s. | n.s. |
| GLIPR1 | 2.6 | n.s. | n.s. | n.s. | n.s. |
| GLIPR1L2 | n.s. | n.s. | 2.2 | n.s. | n.s. |
| GLIS2 | −2.0 | n.s. | n.s. | n.s. | n.s. |
| GLIS3 | n.s. | 2.7 | n.s. | n.s. | n.s. |
| GLS | 3.9 | n.s. | n.s. | n.s. | n.s. |
| GLT8D2 | −2.9 | −2.6 | n.s. | n.s. | n.s. |
| GLT8D4 | −3.9 | n.s. | n.s. | n.s. | n.s. |
| GM2A | n.s. | −2.0 | n.s. | n.s. | n.s. |
| GMEB1 | 1.8 | n.s. | n.s. | n.s. | n.s. |
| GMFB | 1.9 | n.s. | n.s. | n.s. | n.s. |
| GMNN | 3.1 | n.s. | n.s. | n.s. | n.s. |
| GMPPB | 3.5 | 1.8 | n.s. | n.s. | n.s. |
| GMPS | n.s. | 2.0 | n.s. | n.s. | n.s. |
| GNAI1 | −2.2 | n.s. | n.s. | n.s. | n.s. |
| GNAQ | −2.4 | −3.6 | n.s. | n.s. | n.s. |
| GNAT1 | n.s. | n.s. | n.s. | n.s. | −2.7 |
| GNB4 | −1.9 | n.s. | n.s. | n.s. | n.s. |
| GNG2 | 2.1 | n.s. | n.s. | n.s. | n.s. |
| GNPAT | −4.9 | −2.3 | n.s. | n.s. | n.s. |
| GNPDA1 | 1.9 | n.s. | n.s. | n.s. | n.s. |
| GNS | −1.9 | n.s. | n.s. | n.s. | n.s. |
| GOLGA1 | −2.2 | −4.1 | n.s. | n.s. | n.s. |
| GOLGA2 | n.s. | −2.3 | n.s. | n.s. | n.s. |
| GOLGA2LY1 | n.s. | n.s. | n.s. | n.s. | −1.8 |
| GOLGA3 | −2.2 | n.s. | n.s. | n.s. | n.s. |
| GOLGA4 | 2.8 | n.s. | n.s. | n.s. | n.s. |
| GOLGA8A | −2.3 | n.s. | n.s. | n.s. | n.s. |
| GOLGA8B | −2.7 | n.s. | n.s. | n.s. | n.s. |
| GOLGB1 | −2.3 | n.s. | n.s. | n.s. | n.s. |
| GOLT1B | 1.9 | n.s. | n.s. | n.s. | n.s. |
| GOSR2 | 2.1 | −2.1 | n.s. | n.s. | n.s. |
| GPATCH4 | 2.0 | n.s. | n.s. | n.s. | n.s. |
| GPC4 | n.s. | 3.6 | n.s. | n.s. | n.s. |
| GPC6 | −2.0 | n.s. | n.s. | n.s. | n.s. |
| GPER | n.s. | −4.2 | n.s. | n.s. | n.s. |
| GPR1 | −1.8 | n.s. | n.s. | n.s. | n.s. |
| GPR120 | n.s. | n.s. | −5.3 | n.s. | n.s. |
| GPR124 | −2.4 | n.s. | n.s. | n.s. | n.s. |
| GPR132 | n.s. | n.s. | 2.0 | n.s. | 2.1 |
| GPR133 | −12.0 | n.s. | n.s. | n.s. | n.s. |
| GPR137C | 1.9 | n.s. | n.s. | n.s. | n.s. |
| GPR153 | −2.9 | n.s. | n.s. | n.s. | n.s. |
| GPR157 | 2.2 | n.s. | n.s. | n.s. | n.s. |
| GPR161 | −2.6 | −2.4 | n.s. | n.s. | n.s. |
| GPR172A | 2.6 | n.s. | n.s. | n.s. | n.s. |
| GPR177 | 2.9 | 3.1 | n.s. | n.s. | n.s. |
| GPR56 | 15.9 | 5.1 | n.s. | n.s. | n.s. |
| GPRASP1 | −2.7 | n.s. | n.s. | n.s. | n.s. |
| GPRC5A | 3.4 | n.s. | n.s. | n.s. | n.s. |
| GPRC5C | n.s. | n.s. | n.s. | −1.8 | n.s. |
| GPSM2 | −3.0 | −2.2 | n.s. | n.s. | n.s. |
| GPT2 | 3.9 | n.s. | n.s. | n.s. | n.s. |
| GRAMD1B | n.s. | n.s. | n.s. | n.s. | −1.9 |
| GRAMD3 | −2.2 | n.s. | n.s. | n.s. | n.s. |
| GRB10 | 3.0 | 1.8 | n.s. | n.s. | n.s. |
| GRB14 | 13.3 | 4.3 | n.s. | n.s. | n.s. |
| GRB2 | 2.0 | n.s. | n.s. | n.s. | n.s. |
| GRIA1 | n.s. | n.s. | n.s. | n.s. | 1.9 |
| GRIA3 | −2.9 | n.s. | n.s. | n.s. | n.s. |
| GRIK2 | −2.0 | n.s. | n.s. | n.s. | n.s. |
| GRIPAP1 | n.s. | n.s. | −1.9 | n.s. | n.s. |
| GRK5 | −2.5 | n.s. | n.s. | n.s. | n.s. |
| GRLF1 | −2.1 | −2.1 | n.s. | n.s. | n.s. |
| GRM4 | n.s. | n.s. | −2.3 | n.s. | n.s. |
| GRM5 | n.s. | n.s. | −5.7 | n.s. | n.s. |
| GRPEL1 | 2.7 | n.s. | n.s. | n.s. | n.s. |
| GRTP1 | −4.5 | −2.2 | n.s. | n.s. | n.s. |
| GSC2 | n.s. | n.s. | n.s. | n.s. | −6.1 |
| GSDMDC1 | −1.8 | n.s. | n.s. | n.s. | n.s. |
| GSK3B | 1.9 | n.s. | n.s. | n.s. | n.s. |
| GSN | −2.1 | n.s. | n.s. | n.s. | n.s. |
| GSPT1 | 2.0 | n.s. | n.s. | n.s. | n.s. |
| GSR | 1.9 | n.s. | n.s. | n.s. | n.s. |
| GSTM1 | −2.0 | n.s. | n.s. | n.s. | n.s. |
| GSTM2 | −2.6 | n.s. | n.s. | n.s. | n.s. |
| GSTM4 | −2.3 | −1.8 | n.s. | n.s. | n.s. |
| GSTM5 | −2.8 | n.s. | n.s. | n.s. | n.s. |
| GS TO1 | 1.8 | n.s. | n.s. | n.s. | n.s. |
| GTDC1 | −1.9 | n.s. | n.s. | n.s. | n.s. |
| GTF3C4 | −2.1 | −2.5 | n.s. | n.s. | n.s. |
| GTPBP10 | 2.0 | n.s. | n.s. | n.s. | n.s. |
| GTPBP2 | n.s. | −3.8 | n.s. | n.s. | n.s. |
| GTPBP5 | 1.9 | n.s. | n.s. | n.s. | n.s. |
| GTSE1 | 4.2 | n.s. | n.s. | n.s. | n.s. |
| GULP1 | n.s. | −2.6 | n.s. | n.s. | n.s. |
| GUSBL2 | n.s. | −2.4 | n.s. | n.s. | n.s. |
| GYPC | −2.0 | n.s. | n.s. | n.s. | n.s. |
| GYS1 | n.s. | 2.4 | n.s. | n.s. | n.s. |
| H2AFV | n.s. | −1.8 | n.s. | n.s. | n.s. |
| H2AFX | 2.7 | n.s. | n.s. | n.s. | n.s. |
| H2BFS | 4.0 | n.s. | n.s. | n.s. | n.s. |
| H40632 | n.s. | n.s. | n.s. | 4.0 | n.s. |
| H43551 | n.s. | n.s. | 1.9 | 2.0 | 1.9 |
| H6PD | −2.1 | n.s. | n.s. | n.s. | n.s. |
| HAB1 | n.s. | n.s. | n.s. | −2.1 | −1.9 |
| HABP4 | n.s. | n.s. | −1.8 | n.s. | n.s. |
| HACE1 | −2.2 | n.s. | n.s. | n.s. | n.s. |
| HADHA | n.s. | n.s. | −2.1 | n.s. | n.s. |
| HAL | n.s. | n.s. | 5.4 | n.s. | n.s. |
| HAPLN1 | 2.0 | 3.2 | n.s. | n.s. | n.s. |
| HAPLN3 | n.s. | n.s. | n.s. | −2.0 | n.s. |
| HARS | n.s. | n.s. | −1.9 | n.s. | n.s. |
| HAS2 | n.s. | 9.4 | n.s. | n.s. | n.s. |
| HAT1 | 1.9 | n.s. | n.s. | n.s. | n.s. |
| HBEGF | 14.4 | 15.7 | n.s. | n.s. | n.s. |
| HCCS | 2.0 | n.s. | n.s. | n.s. | n.s. |
| HCFC1R1 | −3.8 | −2.2 | n.s. | n.s. | n.s. |
| hCG_1730474 | n.s. | 5.2 | n.s. | n.s. | n.s. |
| hCG_1776047 | n.s. | n.s. | 2.3 | n.s. | n.s. |
| hCG_1806964 | −2.0 | n.s. | n.s. | n.s. | n.s. |
| hCG_1985469 | n.s. | −1.8 | n.s. | n.s. | n.s. |
| hCG_20426 | −2.0 | n.s. | n.s. | n.s. | n.s. |
| HCG11 | −1.8 | −4.6 | n.s. | n.s. | n.s. |
| HCP5 | n.s. | n.s. | −1.8 | n.s. | n.s. |
| HDAC11 | n.s. | n.s. | −1.9 | n.s. | n.s. |
| HDAC4 | −2.1 | n.s. | n.s. | n.s. | n.s. |
| HDAC9 | n.s. | 3.2 | n.s. | n.s. | n.s. |
| HEATR1 | n.s. | 1.9 | n.s. | n.s. | n.s. |
| HEATR5A | −2.7 | −2.0 | n.s. | n.s. | n.s. |
| HECW2 | n.s. | −1.9 | n.s. | n.s. | n.s. |
| HEG1 | −2.0 | n.s. | n.s. | n.s. | n.s. |
| HELLS | 3.0 | 2.6 | n.s. | n.s. | n.s. |
| HELZ | −3.0 | −2.7 | n.s. | n.s. | n.s. |
| HEPH | −2.1 | n.s. | n.s. | n.s. | n.s. |
| HERC3 | n.s. | 1.8 | n.s. | n.s. | n.s. |
| HERPUD2 | n.s. | n.s. | n.s. | −1.8 | n.s. |
| HES1 | 8.1 | 3.9 | 1.9 | n.s. | n.s. |
| HES4 | 10.3 | n.s. | n.s. | n.s. | n.s. |
| HESX1 | n.s. | n.s. | 2.5 | n.s. | n.s. |
| HEXIM12 | n.s. | −2.1 | n.s. | n.s. | n.s. |
| HEY1 | 8.3 | n.s. | n.s. | n.s. | n.s. |
| HFE | −1.9 | −1.8 | n.s. | n.s. | n.s. |
| HFL@ | 5.3 | n.s. | n.s. | n.s. | n.s. |
| HGSNAT | −3.4 | n.s. | n.s. | n.s. | n.s. |
| HHLA3 | −2.1 | n.s. | n.s. | n.s. | n.s. |
| HIBADH | −2.7 | n.s. | n.s. | n.s. | n.s. |
| HIC2 | n.s. | −3.7 | n.s. | n.s. | n.s. |
| HIP1 | 2.0 | n.s. | n.s. | n.s. | n.s. |
| HIP2 | 1.8 | n.s. | n.s. | n.s. | n.s. |
| HIPK2 | 3.7 | n.s. | n.s. | n.s. | n.s. |
| HIRIP3 | 2.2 | n.s. | n.s. | n.s. | n.s. |
| HIST1H1A | n.s. | n.s. | n.s. | n.s. | −1.8 |
| HIST1H1C | 5.4 | n.s. | n.s. | n.s. | n.s. |
| HIST1H1D | 2.8 | n.s. | n.s. | n.s. | n.s. |
| HIST1H1E | 10.0 | n.s. | n.s. | n.s. | n.s. |
| HIST1H2AB | 2.1 | n.s. | n.s. | n.s. | n.s. |
| HIST1H2AD | 10.2 | n.s. | n.s. | n.s. | n.s. |
| HIST1H2AE | 16.0 | 2.2 | n.s. | n.s. | n.s. |
| HIST1H2AG | 2.0 | −2.6 | n.s. | n.s. | n.s. |
| HIST1H2AI | 2.1 | n.s. | n.s. | n.s. | n.s. |
| HIST1H2AM | 3.0 | n.s. | n.s. | n.s. | n.s. |
| HIST1H2BB | 7.9 | n.s. | n.s. | n.s. | n.s. |
| HIST1H2BC | 5.3 | n.s. | n.s. | n.s. | n.s. |
| HIST1H2BD | 6.3 | n.s. | n.s. | n.s. | n.s. |
| HIST1H2BE | 4.4 | n.s. | n.s. | n.s. | n.s. |
| HIST1H2BF | 3.6 | n.s. | n.s. | n.s. | n.s. |
| HIST1H2BG | 41.8 | n.s. | n.s. | n.s. | n.s. |
| HIST1H2BH | 5.1 | n.s. | n.s. | n.s. | n.s. |
| HIST1H2BI | 5.6 | n.s. | n.s. | n.s. | n.s. |
| HIST1H2BJ | 11.1 | n.s. | n.s. | n.s. | n.s. |
| HIST1H2BK | 2.9 | n.s. | n.s. | n.s. | n.s. |
| HIST1H2BM | n.s. | 1.9 | n.s. | n.s. | n.s. |
| HIST1H2BO | 3.4 | n.s. | n.s. | n.s. | n.s. |
| HIST1H3A | 7.2 | n.s. | n.s. | n.s. | n.s. |
| HIST1H3B | 4.1 | n.s. | n.s. | n.s. | n.s. |
| HIST1H3D | 29.4 | n.s. | n.s. | n.s. | n.s. |
| HIST1H3F | 2.3 | n.s. | n.s. | n.s. | n.s. |
| HIST1H3G | 3.2 | n.s. | n.s. | n.s. | n.s. |
| HIST1H3H | 8.1 | −2.5 | n.s. | n.s. | n.s. |
| HIST1H4B | 3.7 | 2.4 | n.s. | n.s. | n.s. |
| HIST1H4C | 2.4 | n.s. | n.s. | n.s. | n.s. |
| HIST1H4D | 3.2 | n.s. | n.s. | n.s. | n.s. |
| HIST1H4E | 7.6 | 2.5 | n.s. | n.s. | n.s. |
| HIST1H4H | 7.2 | n.s. | n.s. | n.s. | n.s. |
| HIST1H4J | 2.6 | n.s. | n.s. | n.s. | n.s. |
| HIST1H4K | 3.5 | n.s. | n.s. | n.s. | n.s. |
| HIST2H2AA | n.s. | −3.0 | n.s. | n.s. | n.s. |
| HIST2H2AA3 | 6.0 | n.s. | n.s. | n.s. | n.s. |
| HIST2H2BE | 4.3 | n.s. | n.s. | n.s. | n.s. |
| HIST2H4 | n.s. | −2.8 | n.s. | n.s. | n.s. |
| HIST3H2A | n.s. | −2.3 | n.s. | n.s. | n.s. |
| HK2 | 6.5 | 2.4 | n.s. | n.s. | n.s. |
| HLCS | −1.9 | n.s. | n.s. | n.s. | n.s. |
| HLX | 2.8 | 1.9 | n.s. | n.s. | n.s. |
| HM13 | 1.8 | n.s. | n.s. | n.s. | n.s. |
| HMBOX1 | −2.1 | −2.1 | n.s. | n.s. | n.s. |
| HMCN1 | −2.5 | n.s. | n.s. | n.s. | n.s. |
| HMG20B | n.s. | −1.9 | n.s. | n.s. | n.s. |
| HMGA2 | 5.3 | 2.4 | n.s. | n.s. | n.s. |
| HMGB1 | 1.8 | n.s. | n.s. | n.s. | n.s. |
| HMGB2 | n.s. | 1.9 | n.s. | n.s. | n.s. |
| HMGB3 | n.s. | −2.3 | n.s. | n.s. | n.s. |
| HMGCR | −2.0 | n.s. | n.s. | n.s. | n.s. |
| HMMR | 7.1 | 4.5 | n.s. | n.s. | n.s. |
| HMOX1 | 34.2 | 6.2 | n.s. | n.s. | n.s. |
| HN1 | 2.7 | n.s. | n.s. | n.s. | n.s. |
| HNMT | −3.1 | n.s. | n.s. | n.s. | n.s. |
| HNRNPA2B1 | −2.6 | −2.1 | n.s. | n.s. | n.s. |
| HNRNPL | 2.3 | n.s. | n.s. | n.s. | n.s. |
| HNRNPR | −2.4 | n.s. | n.s. | n.s. | n.s. |
| HNRNPU | −1.9 | n.s. | n.s. | −1.9 | n.s. |
| HNRPAB | 1.9 | n.s. | n.s. | n.s. | n.s. |
| HNRPD | −5.0 | −1.9 | n.s. | n.s. | n.s. |
| HNRPH3 | n.s. | −2.4 | n.s. | n.s. | n.s. |
| HNRPLL | n.s. | n.s. | n.s. | 1.8 | 1.9 |
| HNT | 1.9 | n.s. | n.s. | n.s. | n.s. |
| HOMER1 | n.s. | 2.5 | n.s. | n.s. | n.s. |
| HOMER2 | 3.1 | n.s. | n.s. | n.s. | n.s. |
| HOOK3 | −1.8 | n.s. | n.s. | n.s. | n.s. |
| HOXA1 | n.s. | −2.1 | n.s. | n.s. | n.s. |
| HOXA10 | n.s. | −2.4 | n.s. | n.s. | n.s. |
| HOXA11 | n.s. | −2.0 | n.s. | n.s. | n.s. |
| HOXA13 | −2.5 | n.s. | n.s. | n.s. | n.s. |
| HOXA2 | 2.4 | n.s. | n.s. | n.s. | n.s. |
| HOXA3 | −3.0 | −2.8 | n.s. | n.s. | n.s. |
| HOXB3 | −2.1 | n.s. | n.s. | n.s. | n.s. |
| HOXB5 | 2.0 | 1.9 | n.s. | n.s. | n.s. |
| HOXB6 | 1.9 | n.s. | n.s. | n.s. | n.s. |
| HOXB7 | 1.9 | n.s. | n.s. | n.s. | n.s. |
| HOXC6 | 3.2 | n.s. | n.s. | n.s. | n.s. |
| HOXC9 | 2.4 | n.s. | n.s. | n.s. | n.s. |
| HOXD4 | −4.2 | −3.0 | n.s. | n.s. | n.s. |
| HOXD9 | n.s. | −2.0 | n.s. | n.s. | n.s. |
| HP1BP3 | −2.6 | n.s. | n.s. | n.s. | n.s. |
| HPCAL1 | n.s. | 1.9 | n.s. | n.s. | n.s. |
| hqp0376 | 2.1 | n.s. | n.s. | n.s. | n.s. |
| protein | | | | | |
| HRAS | 1.9 | n.s. | n.s. | n.s. | n.s. |
| HRASLS | n.s. | n.s. | n.s. | n.s. | 9.2 |
| HS2ST1 | −1.8 | n.s. | n.s. | n.s. | n.s. |
| HS3ST2 | 5.7 | n.s. | n.s. | n.s. | n.s. |
| HS3ST3B1 | −2.3 | −2.8 | n.s. | n.s. | n.s. |
| HSDL2 | −1.8 | n.s. | n.s. | n.s. | n.s. |
| HSP90AB1 | n.s. | n.s. | n.s. | n.s. | −1.8 |
| HSP90AB3P | n.s. | n.s. | n.s. | −1.8 | −1.9 |
| HSPA12A | −2.6 | −2.1 | n.s. | n.s. | n.s. |
| HSPA14 | 2.1 | n.s. | n.s. | n.s. | n.s. |
| HSPA1A | 3.4 | n.s. | n.s. | n.s. | n.s. |
| HSPA1B | 4.4 | n.s. | n.s. | n.s. | n.s. |
| HSPA2 | 2.5 | n.s. | n.s. | n.s. | n.s. |
| HSPA5 | 2.5 | 2.0 | n.s. | n.s. | n.s. |
| HSPA6 | 28.0 | 2.2 | −2.1 | n.s. | n.s. |
| HSPB3 | −3.1 | −2.7 | n.s. | n.s. | n.s. |
| HSPB7 | 2.4 | n.s. | n.s. | n.s. | n.s. |
| HSPB8 | 4.9 | n.s. | n.s. | n.s. | n.s. |
| HSPC047 | −2.0 | n.s. | n.s. | n.s. | n.s. |
| HSPC111 | 3.1 | 2.9 | n.s. | n.s. | n.s. |
| HSPC171 | 1.9 | n.s. | n.s. | n.s. | n.s. |
| HSPC173 | 2.3 | n.s. | n.s. | n.s. | n.s. |
| HSPC180 | n.s. | 1.9 | n.s. | n.s. | n.s. |
| HSPC252 | n.s. | −2.5 | n.s. | n.s. | n.s. |
| HSPD1 | 2.1 | 2.1 | n.s. | n.s. | n.s. |
| HSPG2 | n.s. | 2.0 | n.s. | n.s. | n.s. |
| HSPH1 | 2.5 | 2.3 | n.s. | n.s. | n.s. |
| HTATIP2 | −1.8 | −2.1 | n.s. | n.s. | n.s. |
| HTR2A | 2.0 | n.s. | n.s. | n.s. | n.s. |
| HTR7 | 3.5 | n.s. | n.s. | n.s. | n.s. |
| HUNK | −12.8 | n.s. | n.s. | n.s. | n.s. |
| HUS1 | 1.8 | n.s. | n.s. | n.s. | n.s. |
| HUWE1 | n.s. | 2.2 | n.s. | n.s. | n.s. |
| HYLS1 | n.s. | −1.9 | n.s. | n.s. | n.s. |
| HYOU1 | 3.1 | 2.6 | n.s. | n.s. | n.s. |
| HYPE | 2.7 | n.s. | n.s. | n.s. | n.s. |
| IAH1 | n.s. | −1.8 | n.s. | n.s. | n.s. |
| IARS | 2.7 | n.s. | n.s. | n.s. | n.s. |
| IBRDC3 | 3.1 | n.s. | n.s. | n.s. | n.s. |
| ICA1L | −2.8 | −2.0 | n.s. | n.s. | n.s. |
| ICK | n.s. | −2.1 | n.s. | n.s. | n.s. |
| ID1 | 9.5 | n.s. | n.s. | n.s. | n.s. |
| ID2 | 2.4 | n.s. | n.s. | n.s. | n.s. |
| ID3 | 28.5 | 1.8 | n.s. | n.s. | n.s. |
| ID4 | 15.5 | 2.8 | n.s. | n.s. | n.s. |
| IDH1 | −2.0 | −1.8 | n.s. | n.s. | n.s. |
| IDH3A | 2.2 | 1.9 | n.s. | n.s. | n.s. |
| IDH3B | n.s. | n.s. | −1.9 | n.s. | n.s. |
| IDS | −1.9 | n.s. | n.s. | n.s. | n.s. |
| IER2 | 1.8 | 2.0 | n.s. | n.s. | n.s. |
| IER3 | 3.0 | 3.0 | n.s. | n.s. | n.s. |
| IFI27 | 2.0 | n.s. | n.s. | n.s. | n.s. |
| IFIT1 | −9.1 | −14.3 | n.s. | n.s. | n.s. |
| IFIT2 | −3.8 | n.s. | n.s. | n.s. | n.s. |
| IFIT3 | n.s. | −2.3 | −2.1 | n.s. | n.s. |
| IFNAR1 | 2.3 | n.s. | n.s. | n.s. | n.s. |
| IFRD2 | 3.9 | n.s. | n.s. | n.s. | n.s. |
| IFT52 | −1.9 | n.s. | n.s. | n.s. | n.s. |
| IFT57 | −2.3 | n.s. | n.s. | n.s. | n.s. |
| IFT74 | n.s. | −1.8 | n.s. | n.s. | n.s. |
| IFT80 | −2.1 | −1.9 | n.s. | n.s. | n.s. |
| IFT81 | −2.9 | n.s. | n.s. | n.s. | n.s. |
| IGF1R | −2.7 | n.s. | n.s. | n.s. | n.s. |
| IGFBP5 | −1.8 | n.s. | n.s. | n.s. | n.s. |
| IGHG1 | n.s. | n.s. | −3.0 | n.s. | n.s. |
| IGJ | −2.6 | n.s. | n.s. | n.s. | n.s. |
| IGLL1 | n.s. | n.s. | n.s. | −2.7 | n.s. |
| IGSF9 | n.s. | n.s. | n.s. | n.s. | 1.9 |
| IHPK2 | n.s. | −2.1 | n.s. | n.s. | n.s. |
| IKZF2 | −3.3 | n.s. | n.s. | n.s. | n.s. |
| IKZF4 | −2.3 | −2.9 | 1.9 | n.s. | n.s. |
| IKZF5 | 2.8 | n.s. | n.s. | n.s. | n.s. |
| IL11 | 10.4 | 10.6 | n.s. | n.s. | n.s. |
| IL15 | n.s. | −2.2 | n.s. | n.s. | n.s. |
| IL17RB | −3.8 | −2.4 | n.s. | n.s. | n.s. |
| IL17RC | n.s. | 1.9 | n.s. | n.s. | n.s. |
| IL17RD | −6.7 | n.s. | n.s. | n.s. | n.s. |
| IL1R1 | n.s. | 2.9 | n.s. | n.s. | n.s. |
| IL1RAP | n.s. | n.s. | n.s. | n.s. | 1.9 |
| IL1RN | 4.5 | 2.2 | n.s. | n.s. | n.s. |
| IL2 | n.s. | n.s. | n.s. | n.s. | 2.2 |
| IL20RB | −5.2 | −2.6 | n.s. | n.s. | n.s. |
| IL21R | n.s. | 5.1 | n.s. | n.s. | n.s. |
| IL24 | 3.2 | n.s. | n.s. | n.s. | n.s. |
| IL27RA | n.s. | n.s. | n.s. | n.s. | 1.9 |
| IL32 | 18.2 | 2.4 | n.s. | n.s. | n.s. |
| IL33 | 72.6 | 10.9 | n.s. | n.s. | n.s. |
| IL4R | n.s. | 3.3 | n.s. | n.s. | n.s. |
| IL6 | n.s. | 2.6 | n.s. | n.s. | n.s. |
| IL6R | 2.0 | n.s. | n.s. | n.s. | n.s. |
| IL6ST | −1.9 | n.s. | n.s. | n.s. | n.s. |
| IL7 | −2.3 | n.s. | n.s. | n.s. | n.s. |
| IL7R | −2.0 | n.s. | n.s. | n.s. | n.s. |
| IL8 | 21.5 | 14.8 | n.s. | n.s. | n.s. |
| ILF3 | 1.8 | 1.9 | n.s. | n.s. | n.s. |
| IMMP1L | 2.0 | n.s. | n.s. | n.s. | n.s. |
| IMMT | 2.9 | n.s. | n.s. | n.s. | n.s. |
| IMP4 | 2.3 | n.s. | n.s. | n.s. | n.s. |
| IMPACT | −2.0 | n.s. | n.s. | n.s. | n.s. |
| IMPAD1 | 2.2 | n.s. | n.s. | n.s. | n.s. |
| INADL | −5.3 | −2.3 | n.s. | n.s. | n.s. |
| ING1 | 3.4 | n.s. | n.s. | n.s. | n.s. |
| ING4 | −1.9 | −2.0 | n.s. | n.s. | n.s. |
| INHBA | 5.0 | 4.4 | n.s. | n.s. | n.s. |
| INHBB | −2.9 | n.s. | n.s. | n.s. | n.s. |
| INPP4B | n.s. | 1.9 | n.s. | n.s. | n.s. |
| INSIG1 | n.s. | 2.1 | n.s. | n.s. | n.s. |
| INTS10 | n.s. | −2.5 | n.s. | n.s. | n.s. |
| INTS6 | −1.8 | n.s. | n.s. | n.s. | n.s. |
| IPO4 | n.s. | 2.2 | n.s. | n.s. | n.s. |
| IPO9 | n.s. | −2.0 | n.s. | n.s. | n.s. |
| IQCE | −1.9 | −1.8 | n.s. | n.s. | n.s. |
| IQGAP3 | n.s. | −2.0 | n.s. | n.s. | n.s. |
| IQSEC1 | −2.1 | n.s. | n.s. | n.s. | n.s. |
| IRAK1BP1 | −3.3 | n.s. | n.s. | n.s. | n.s. |
| IRAK2 | n.s. | 1.9 | n.s. | n.s. | n.s. |
| IREB2 | −1.9 | −1.8 | n.s. | n.s. | n.s. |
| IRF2 | −2.1 | n.s. | n.s. | n.s. | n.s. |
| IRF2BP2 | n.s. | 2.1 | n.s. | n.s. | n.s. |
| IRS1 | −2.0 | n.s. | n.s. | n.s. | n.s. |
| ISG20 | 1.9 | 2.2 | n.s. | n.s. | n.s. |
| ISG20L1 | 1.8 | 1.8 | n.s. | n.s. | n.s. |
| ITCH | −2.0 | n.s. | n.s. | n.s. | n.s. |
| ITGA2 | 4.5 | 2.4 | n.s. | n.s. | n.s. |
| ITGA3 | 2.2 | 1.8 | n.s. | n.s. | n.s. |
| ITGA4 | −4.4 | n.s. | n.s. | n.s. | n.s. |
| ITGA5 | 2.3 | 2.0 | n.s. | n.s. | n.s. |
| ITGA6 | 2.7 | n.s. | n.s. | n.s. | n.s. |
| ITGAV | −2.1 | n.s. | n.s. | n.s. | n.s. |
| ITGB2 | n.s. | n.s. | n.s. | n.s. | 2.0 |
| ITGB3 | 3.1 | n.s. | n.s. | n.s. | n.s. |
| ITGB8 | −5.4 | −3.7 | n.s. | n.s. | n.s. |
| ITGBL1 | −1.9 | n.s. | n.s. | n.s. | n.s. |
| ITIH5 | −2.6 | n.s. | n.s. | n.s. | n.s. |
| ITPR2 | −3.5 | n.s. | n.s. | n.s. | n.s. |
| ITPR3 | 2.3 | 2.6 | n.s. | n.s. | n.s. |
| ITSN1 | −1.8 | n.s. | n.s. | n.s. | n.s. |
| ITSN2 | n.s. | −1.8 | n.s. | n.s. | n.s. |
| JARID1A | −1.9 | −1.9 | n.s. | n.s. | n.s. |
| JARID1C | −2.4 | n.s. | n.s. | n.s. | n.s. |
| JARID2 | 2.4 | n.s. | n.s. | n.s. | n.s. |
| JAZF1 | −2.0 | n.s. | n.s. | n.s. | n.s. |
| JHDM1D | 1.9 | n.s. | n.s. | n.s. | n.s. |
| JMJD2C | −2.5 | n.s. | n.s. | n.s. | n.s. |
| JMJD3 | 2.7 | n.s. | n.s. | n.s. | n.s. |
| JMJD4 | 2.3 | 1.9 | n.s. | n.s. | n.s. |
| JMJD6 | 1.9 | 1.9 | n.s. | n.s. | n.s. |
| JMY | 2.3 | −2.2 | n.s. | n.s. | n.s. |
| JRK | n.s. | −2.6 | n.s. | n.s. | n.s. |
| JUB | −1.8 | n.s. | n.s. | n.s. | n.s. |
| JUN | 2.7 | 5.3 | n.s. | n.s. | n.s. |
| JUNB | 3.0 | 5.9 | n.s. | n.s. | n.s. |
| JUP | −9.2 | n.s. | n.s. | n.s. | n.s. |
| KATNAL2 | n.s. | n.s. | 2.0 | n.s. | 2.7 |
| KBTBD2 | 1.8 | n.s. | n.s. | n.s. | n.s. |
| KBTBD3 | n.s. | −2.1 | n.s. | n.s. | n.s. |
| KBTBD7 | −1.9 | −3.3 | n.s. | n.s. | n.s. |
| KBTBD8 | 5.2 | n.s. | n.s. | n.s. | n.s. |
| KCNAB1 | n.s. | −2.2 | n.s. | n.s. | n.s. |
| KCNC4 | 4.1 | 2.2 | n.s. | n.s. | n.s. |
| KCNE1 | n.s. | n.s. | n.s. | 3.9 | n.s. |
| KCNE3 | n.s. | n.s. | 3.5 | n.s. | 4.0 |
| KCNE4 | 2.6 | n.s. | n.s. | n.s. | n.s. |
| KCNG1 | 2.3 | 2.2 | n.s. | n.s. | n.s. |
| KCNH2 | n.s. | n.s. | n.s. | n.s. | 2.6 |
| KCNJ15 | n.s. | 7.8 | n.s. | n.s. | n.s. |
| KCNJ2 | −2.2 | −2.0 | n.s. | n.s. | n.s. |
| KCNK1 | 8.9 | n.s. | n.s. | n.s. | n.s. |
| KCNK2 | −2.2 | n.s. | n.s. | n.s. | n.s. |
| KCNMB2 | n.s. | n.s. | n.s. | n.s. | 2.8 |
| KCNN4 | 3.5 | 2.1 | n.s. | n.s. | n.s. |
| KCNQ5 | n.s. | 2.6 | n.s. | n.s. | n.s. |
| KCNS2 | −4.1 | n.s. | n.s. | n.s. | n.s. |
| KCNS3 | n.s. | 2.0 | n.s. | n.s. | n.s. |
| KCTD11 | −1.9 | n.s. | n.s. | n.s. | n.s. |
| KCTD12 | −3.1 | n.s. | n.s. | n.s. | n.s. |
| KCTD14 | 2.5 | n.s. | n.s. | n.s. | n.s. |
| KCTD17 | n.s. | n.s. | −1.8 | n.s. | n.s. |
| KCTD4 | −2.4 | n.s. | n.s. | n.s. | n.s. |
| KCTD5 | 2.1 | n.s. | n.s. | n.s. | n.s. |
| KGFLP1 | n.s. | 2.1 | n.s. | n.s. | n.s. |
| KIAA0020 | n.s. | 1.8 | n.s. | n.s. | n.s. |
| KIAA0090 | n.s. | 1.9 | n.s. | n.s. | n.s. |
| KIAA0101 | 1.8 | n.s. | n.s. | n.s. | n.s. |
| KIAA0182 | −2.4 | n.s. | n.s. | n.s. | n.s. |
| KIAA0194 | 6.5 | n.s. | n.s. | n.s. | n.s. |
| KIAA0226 | n.s. | n.s. | n.s. | 2.1 | n.s. |
| KIAA0232 | −2.2 | −2.2 | n.s. | n.s. | n.s. |
| KIAA0241 | 2.7 | n.s. | n.s. | n.s. | n.s. |
| KIAA0247 | 1.8 | n.s. | n.s. | n.s. | n.s. |
| KIAA0256 | n.s. | −1.9 | n.s. | n.s. | n.s. |
| KIAA0265 | n.s. | −1.8 | n.s. | n.s. | n.s. |
| KIAA0280 | −1.8 | n.s. | n.s. | n.s. | n.s. |
| KIAA0372 | −2.2 | n.s. | n.s. | n.s. | n.s. |
| KIAA0409 | 2.0 | n.s. | n.s. | n.s. | n.s. |
| KIAA0427 | −2.0 | n.s. | n.s. | n.s. | n.s. |
| KIAA0513 | n.s. | −3.2 | n.s. | n.s. | n.s. |
| KIAA0528 | −2.0 | n.s. | n.s. | n.s. | n.s. |
| KIAA0556 | 5.5 | n.s. | n.s. | n.s. | n.s. |
| KIAA0664 | 1.9 | 2.2 | n.s. | n.s. | n.s. |
| KIAA0802 | −3.1 | −2.5 | n.s. | n.s. | n.s. |
| KIAA0892 | 2.0 | n.s. | n.s. | n.s. | n.s. |
| KIAA0913 | n.s. | n.s. | −2.1 | n.s. | n.s. |
| KIAA0922 | −6.7 | n.s. | n.s. | n.s. | n.s. |
| KIAA0974 | −2.4 | n.s. | n.s. | n.s. | n.s. |
| KIAA0999 | −2.5 | −2.4 | n.s. | n.s. | n.s. |
| KIAA1107 | −1.8 | n.s. | n.s. | n.s. | n.s. |
| KIAA1109 | −2.4 | n.s. | n.s. | n.s. | n.s. |
| KIAA1143 | −1.8 | n.s. | n.s. | n.s. | n.s. |
| KIAA1199 | −4.9 | n.s. | n.s. | n.s. | n.s. |
| KIAA1217 | n.s. | n.s. | n.s. | 1.8 | n.s. |
| KIAA1267 | n.s. | 2.4 | n.s. | n.s. | n.s. |
| KIAA1276 | −2.6 | n.s. | n.s. | n.s. | n.s. |
| KIAA1305 | −6.0 | −3.2 | n.s. | n.s. | n.s. |
| KIAA1333 | n.s. | −2.0 | n.s. | 3.7 | n.s. |
| KIAA1370 | n.s. | −1.8 | n.s. | n.s. | n.s. |
| KIAA1377 | −2.3 | −2.5 | 1.9 | n.s. | n.s. |
| KIAA1407 | −2.5 | −2.1 | n.s. | n.s. | n.s. |
| KIAA1430 | −2.4 | n.s. | n.s. | n.s. | n.s. |
| KIAA1432 | n.s. | n.s. | n.s. | n.s. | 1.9 |
| KIAA1450 | 2.9 | n.s. | n.s. | n.s. | n.s. |
| KIAA1462 | −3.0 | n.s. | n.s. | n.s. | n.s. |
| KIAA1467 | n.s. | −1.8 | n.s. | n.s. | n.s. |
| KIAA1524 | n.s. | n.s. | n.s. | 1.8 | n.s. |
| KIAA1545 | −2.9 | n.s. | n.s. | n.s. | n.s. |
| KIAA1546 | −1.9 | −2.0 | n.s. | n.s. | n.s. |
| KIAA1549 | −2.9 | −2.2 | n.s. | n.s. | n.s. |
| KIAA1598 | −2.0 | n.s. | n.s. | n.s. | n.s. |
| KIAA1609 | n.s. | 1.8 | n.s. | n.s. | n.s. |
| KIAA1632 | n.s. | 3.0 | n.s. | n.s. | n.s. |
| KIAA1641 | n.s. | 1.9 | n.s. | n.s. | n.s. |
| KIAA1648 | n.s. | −2.4 | n.s. | n.s. | n.s. |
| KIAA1683 | 14.9 | n.s. | n.s. | n.s. | n.s. |
| KIAA1704 | −2.3 | n.s. | n.s. | n.s. | n.s. |
| KIAA1715 | −2.0 | −1.9 | −2.4 | −1.9 | n.s. |
| KIAA1729 | −1.8 | −1.9 | n.s. | n.s. | n.s. |
| KIAA1731 | n.s. | 2.8 | n.s. | n.s. | n.s. |
| KIAA1751 | n.s. | n.s. | −3.4 | −11.0 | −5.2 |
| KIAA1754 | 4.6 | 2.5 | n.s. | n.s. | n.s. |
| KIAA1799 | n.s. | −2.4 | n.s. | n.s. | n.s. |
| KIAA1833 | 2.8 | n.s. | n.s. | n.s. | n.s. |
| KIAA1908 | −3.5 | n.s. | n.s. | n.s. | n.s. |
| KIAA1913 | n.s. | 1.8 | n.s. | n.s. | n.s. |
| KIAA1919 | 2.4 | 2.5 | n.s. | n.s. | n.s. |
| KIAA1946 | −1.8 | −2.0 | n.s. | n.s. | n.s. |
| KIAA1958 | −2.5 | −3.2 | n.s. | n.s. | n.s. |
| KIAA1967 | n.s. | −3.7 | n.s. | n.s. | n.s. |
| KIAA2018 | −1.8 | −3.1 | n.s. | n.s. | n.s. |
| KIF11 | 2.6 | n.s. | n.s. | n.s. | n.s. |
| KIF14 | 2.0 | −3.2 | n.s. | n.s. | n.s. |
| KIF15 | 2.1 | n.s. | n.s. | n.s. | n.s. |
| KIF18A | 3.0 | n.s. | n.s. | n.s. | n.s. |
| KIF20A | n.s. | −2.9 | n.s. | n.s. | n.s. |
| KIF22 | 2.8 | −2.7 | n.s. | n.s. | n.s. |
| KIF23 | 2.8 | n.s. | n.s. | n.s. | n.s. |
| KIF24 | 2.4 | n.s. | n.s. | n.s. | n.s. |
| KIF26A | −5.2 | n.s. | n.s. | n.s. | n.s. |
| KIF2A | −2.6 | n.s. | n.s. | n.s. | n.s. |
| KIF2C | 4.0 | n.s. | n.s. | n.s. | n.s. |
| KIF3A | n.s. | −2.1 | n.s. | n.s. | n.s. |
| KIF3B | n.s. | −1.9 | n.s. | n.s. | n.s. |
| KIF4A | 3.5 | n.s. | −1.8 | n.s. | n.s. |
| KIF5A | n.s. | −1.9 | n.s. | n.s. | n.s. |
| KIF6 | −5.9 | n.s. | n.s. | n.s. | n.s. |
| KIFC1 | 3.5 | n.s. | n.s. | n.s. | n.s. |
| KIFC3 | n.s. | 2.4 | n.s. | n.s. | n.s. |
| KIR3DL1 | n.s. | n.s. | 3.0 | n.s. | n.s. |
| KIRREL3 | n.s. | 2.9 | n.s. | n.s. | n.s. |
| KIT | −87.7 | n.s. | n.s. | n.s. | n.s. |
| KITLG | −2.5 | n.s. | n.s. | n.s. | n.s. |
| KLC1 | 2.5 | n.s. | n.s. | n.s. | n.s. |
| KLC3 | n.s. | 2.0 | n.s. | n.s. | −2.2 |
| KLC4 | n.s. | n.s. | n.s. | −2.1 | n.s. |
| KLF1 | n.s. | n.s. | n.s. | −2.1 | n.s. |
| KLF10 | 2.1 | 1.9 | n.s. | n.s. | n.s. |
| KLF12 | −2.4 | −2.3 | n.s. | n.s. | n.s. |
| KLF16 | n.s. | 2.7 | n.s. | n.s. | n.s. |
| KLF2 | n.s. | 2.5 | n.s. | n.s. | n.s. |
| KLF7 | 3.3 | n.s. | n.s. | n.s. | n.s. |
| KLF8 | n.s. | n.s. | n.s. | n.s. | 2.1 |
| KLHDC1 | −2.3 | n.s. | n.s. | n.s. | n.s. |
| KLHDC4 | n.s. | n.s. | −2.4 | n.s. | n.s. |
| KLHDC5 | 1.9 | 1.8 | n.s. | n.s. | n.s. |
| KLHDC8B | n.s. | −2.1 | −2.1 | n.s. | −1.8 |
| KLHDC9 | −4.2 | −3.5 | n.s. | n.s. | n.s. |
| KLHL11 | n.s. | −1.8 | n.s. | n.s. | n.s. |
| KLHL17 | n.s. | n.s. | −2.1 | −1.9 | −2.0 |
| KLHL18 | 2.3 | n.s. | n.s. | n.s. | n.s. |
| KLHL20 | 7.1 | −1.9 | n.s. | n.s. | n.s. |
| KLHL21 | 15.1 | 2.6 | n.s. | n.s. | n.s. |
| KLHL24 | 10.5 | −2.2 | n.s. | n.s. | n.s. |
| KLHL28 | n.s. | −1.9 | n.s. | n.s. | n.s. |
| KLHL5 | −2.9 | −2.0 | n.s. | n.s. | n.s. |
| KLHL7 | 1.9 | n.s. | n.s. | n.s. | n.s. |
| KPNA4 | 2.3 | n.s. | n.s. | n.s. | n.s. |
| KPNA5 | −2.2 | −2.1 | n.s. | n.s. | n.s. |
| KREMEN1 | −1.9 | n.s. | n.s. | n.s. | n.s. |
| KRIT1 | −2.1 | −2.7 | n.s. | 4.1 | n.s. |
| KRT14 | 3.5 | n.s. | n.s. | n.s. | n.s. |
| KRT15 | 6.9 | n.s. | n.s. | n.s. | n.s. |
| KRT16 | n.s. | n.s. | −1.9 | n.s. | n.s. |
| KRT18 | 4.0 | 2.5 | n.s. | n.s. | n.s. |
| KRT19 | 2.5 | n.s. | n.s. | n.s. | n.s. |
| KRT33A | 20.9 | 3.3 | n.s. | n.s. | n.s. |
| KRT33B | 9.4 | n.s. | n.s. | n.s. | n.s. |
| KRT34 | 12.6 | 4.5 | n.s. | n.s. | n.s. |
| KRT7 | 2.8 | n.s. | n.s. | n.s. | n.s. |
| KRT73 | 3.3 | 6.3 | n.s. | n.s. | n.s. |
| KRT78 | 2.2 | n.s. | n.s. | n.s. | n.s. |
| KRT80 | n.s. | n.s. | n.s. | 2.1 | n.s. |
| KRT81 | 5.9 | n.s. | n.s. | n.s. | n.s. |
| KRTAP1-5 | −2.3 | n.s. | n.s. | n.s. | n.s. |
| KRTAP4-10 | n.s. | n.s. | n.s. | −4.6 | n.s. |
| Kua | 2.0 | n.s. | n.s. | n.s. | n.s. |
| KY | −4.3 | n.s. | n.s. | n.s. | n.s. |
| L3MBTL | −4.7 | n.s. | n.s. | n.s. | n.s. |
| LAMA2 | −2.1 | n.s. | n.s. | n.s. | n.s. |
| LAMA4 | −1.9 | −1.9 | n.s. | n.s. | n.s. |
| LAMB1 | −2.1 | n.s. | n.s. | n.s. | n.s. |
| LAMC2 | n.s. | 3.6 | n.s. | n.s. | n.s. |
| LARP2 | −3.5 | n.s. | n.s. | n.s. | n.s. |
| LARP5 | n.s. | −2.0 | n.s. | n.s. | n.s. |
| LARS | n.s. | n.s. | n.s. | n.s. | −1.9 |
| LASS6 | 2.9 | 2.1 | n.s. | n.s. | n.s. |
| LBA1 | −2.8 | n.s. | n.s. | n.s. | n.s. |
| LBH | n.s. | 1.9 | n.s. | n.s. | n.s. |
| LCAT | −3.0 | n.s. | n.s. | n.s. | n.s. |
| LCE3D | 2.7 | 2.5 | n.s. | n.s. | n.s. |
| LCORL | −2.1 | n.s. | 2.3 | n.s. | n.s. |
| LDB1 | 1.8 | n.s. | n.s. | n.s. | n.s. |
| LDB2 | −2.8 | −2.0 | n.s. | n.s. | n.s. |
| LDLR | n.s. | 4.2 | n.s. | n.s. | n.s. |
| LENG8 | −2.0 | n.s. | n.s. | n.s. | n.s. |
| LEPR | −2.4 | −2.1 | n.s. | n.s. | n.s. |
| LEPREL1 | 11.8 | 7.6 | n.s. | n.s. | n.s. |
| LETM1 | n.s. | 2.1 | n.s. | n.s. | n.s. |
| LETM2 | 2.7 | 2.5 | n.s. | n.s. | n.s. |
| LETMD1 | −2.2 | −1.9 | n.s. | n.s. | n.s. |
| LFNG | n.s. | 2.1 | n.s. | n.s. | n.s. |
| LGALS8 | 3.0 | n.s. | n.s. | n.s. | n.s. |
| LGALS9 | n.s. | n.s. | −2.1 | n.s. | n.s. |
| LGI2 | n.s. | n.s. | 2.9 | 4.2 | n.s. |
| LGI4 | n.s. | n.s. | n.s. | −2.0 | n.s. |
| LGR4 | n.s. | −2.3 | n.s. | n.s. | n.s. |
| LHB | 3.5 | n.s. | n.s. | n.s. | n.s. |
| LHCGR | −4.1 | −3.1 | n.s. | n.s. | n.s. |
| LHX9 | −4.2 | n.s. | n.s. | n.s. | n.s. |
| LIFR | −3.2 | −1.8 | n.s. | n.s. | n.s. |
| LIG1 | 4.6 | 2.4 | n.s. | n.s. | n.s. |
| LIMA1 | n.s. | −2.0 | n.s. | n.s. | n.s. |
| LIMCH1 | −2.0 | n.s. | n.s. | n.s. | n.s. |
| LIMK1 | n.s. | 3.0 | n.s. | n.s. | n.s. |
| LIMS1 | n.s. | 2.4 | n.s. | n.s. | n.s. |
| LIMS2 | −2.3 | n.s. | n.s. | n.s. | n.s. |
| LIMS3 | 2.9 | 3.0 | n.s. | n.s. | n.s. |
| LIN28B | n.s. | n.s. | n.s. | n.s. | 29.6 |
| LIN7B | 1.8 | n.s. | n.s. | n.s. | n.s. |
| LIN9 | 2.3 | n.s. | n.s. | n.s. | n.s. |
| LIPC | −28.8 | n.s. | n.s. | n.s. | n.s. |
| LIPT1 | n.s. | −1.8 | n.s. | n.s. | n.s. |
| LIX1L | −1.9 | n.s. | n.s. | n.s. | n.s. |
| LMCD1 | 4.8 | 3.9 | n.s. | n.s. | n.s. |
| LMNA | −2.6 | n.s. | n.s. | n.s. | n.s. |
| LMO4 | 3.9 | 2.1 | n.s. | n.s. | n.s. |
| LMOD1 | n.s. | −3.7 | n.s. | n.s. | n.s. |
| LNPEP | −2.3 | −1.8 | n.s. | n.s. | n.s. |
| LNX1 | −2.6 | n.s. | n.s. | n.s. | n.s. |
| LOC100049076 | n.s. | −2.2 | n.s. | n.s. | n.s. |
| LOC113179 | 2.0 | n.s. | n.s. | n.s. | n.s. |
| LOC128977 | n.s. | n.s. | n.s. | 1.9 | n.s. |
| LOC130074 | 2.3 | n.s. | n.s. | n.s. | n.s. |
| LOC137886 | −2.3 | n.s. | n.s. | n.s. | n.s. |
| LOC144874 | −2.3 | n.s. | n.s. | n.s. | n.s. |
| LOC146346 | −3.2 | n.s. | n.s. | n.s. | n.s. |
| LOC146909 | 2.1 | n.s. | n.s. | n.s. | n.s. |
| LOC147343 | n.s. | n.s. | 2.8 | n.s. | n.s. |
| LOC147650 | −2.8 | −1.9 | n.s. | n.s. | n.s. |
| LOC147727 | n.s. | −1.8 | n.s. | n.s. | n.s. |
| LOC149478 | −2.7 | n.s. | n.s. | n.s. | n.s. |
| LOC149773 | 2.6 | n.s. | n.s. | n.s. | n.s. |
| LOC151162 | n.s. | 1.9 | n.s. | n.s. | n.s. |
| LOC152217 | 2.2 | n.s. | 3.0 | n.s. | n.s. |
| LOC152485 | 2.0 | 1.8 | n.s. | n.s. | n.s. |
| LOC152742 | −1.8 | n.s. | n.s. | n.s. | n.s. |
| LOC153222 | n.s. | −2.4 | n.s. | n.s. | n.s. |
| LOC153457 | n.s. | n.s. | n.s. | n.s. | 1.9 |
| LOC153682 | −2.7 | −1.8 | n.s. | n.s. | n.s. |
| LOC158257 | n.s. | −9.0 | n.s. | n.s. | n.s. |
| LOC158402 | −2.1 | n.s. | n.s. | n.s. | n.s. |
| LOC201164 | 2.3 | n.s. | n.s. | n.s. | n.s. |
| LOC201175 | 2.8 | n.s. | n.s. | n.s. | n.s. |
| LOC201229 | −2.4 | n.s. | n.s. | n.s. | n.s. |
| LOC201895 | −1.8 | −2.3 | n.s. | n.s. | n.s. |
| LOC203107 | n.s. | −2.3 | n.s. | n.s. | n.s. |
| LOC220077 | n.s. | n.s. | −2.0 | n.s. | n.s. |
| LOC220594 | −1.9 | n.s. | n.s. | n.s. | n.s. |
| LOC220729 | n.s. | n.s. | −1.8 | n.s. | n.s. |
| LOC221091 | −5.8 | n.s. | n.s. | n.s. | n.s. |
| LOC221710 | 2.5 | n.s. | n.s. | n.s. | n.s. |
| LOC222070 | −1.9 | n.s. | n.s. | n.s. | n.s. |
| LOC222159 | −3.0 | n.s. | n.s. | n.s. | n.s. |
| LOC23117 | n.s. | 1.9 | n.s. | n.s. | n.s. |
| LOC253039 | −3.4 | −2.6 | n.s. | n.s. | n.s. |
| LOC254128 | n.s. | −2.5 | n.s. | n.s. | n.s. |
| LOC255480 | −2.0 | −2.0 | n.s. | n.s. | n.s. |
| LOC255512 | n.s. | −2.0 | n.s. | n.s. | n.s. |
| LOC257396 | −3.3 | n.s. | n.s. | n.s. | n.s. |
| LOC25845 | 1.8 | n.s. | n.s. | n.s. | n.s. |
| LOC283075 | −2.1 | −2.3 | n.s. | n.s. | n.s. |
| LOC283357 | −3.5 | −3.1 | n.s. | n.s. | n.s. |
| LOC283378 | n.s. | −2.1 | n.s. | n.s. | n.s. |
| LOC283508 | −1.8 | n.s. | n.s. | n.s. | n.s. |
| LOC283551 | 3.4 | n.s. | n.s. | n.s. | n.s. |
| LOC283658 | −4.0 | n.s. | n.s. | n.s. | n.s. |
| LOC283666 | −2.5 | n.s. | n.s. | n.s. | n.s. |
| LOC283788 | −2.6 | −1.9 | n.s. | n.s. | n.s. |
| LOC283871 | 2.9 | n.s. | n.s. | n.s. | n.s. |
| LOC283874 | −2.5 | n.s. | n.s. | n.s. | n.s. |
| LOC283951 | 2.8 | n.s. | n.s. | n.s. | n.s. |
| LOC284058 | n.s. | 3.7 | n.s. | n.s. | n.s. |
| LOC284072 | 4.4 | n.s. | n.s. | n.s. | n.s. |
| LOC284323 | −2.4 | −2.6 | n.s. | n.s. | n.s. |
| LOC284356 | −2.4 | −2.7 | n.s. | n.s. | n.s. |
| LOC284371 | −2.0 | −2.0 | n.s. | n.s. | n.s. |
| LOC284801 | 4.6 | 3.3 | n.s. | n.s. | n.s. |
| LOC285086 | −2.8 | n.s. | n.s. | n.s. | n.s. |
| LOC285535 | −2.2 | n.s. | n.s. | n.s. | n.s. |
| LOC285550 | −2.6 | n.s. | n.s. | n.s. | n.s. |
| LOC285831 | −2.2 | −1.9 | n.s. | n.s. | n.s. |
| LOC285835 | −4.0 | −1.8 | n.s. | n.s. | n.s. |
| LOC285923 | n.s. | n.s. | −1.9 | n.s. | −1.9 |
| LOC286044 | −2.1 | n.s. | n.s. | n.s. | n.s. |
| LOC286052 | −1.8 | −1.9 | n.s. | n.s. | n.s. |
| LOC286144 | −2.3 | −2.1 | n.s. | n.s. | n.s. |
| LOC286161 | −2.7 | n.s. | n.s. | n.s. | n.s. |
| LOC286167 | 2.3 | 3.3 | n.s. | n.s. | n.s. |
| LOC286170 | −1.9 | n.s. | n.s. | n.s. | n.s. |
| LOC286437 | −2.2 | −3.1 | n.s. | n.s. | n.s. |
| LOC338328 | n.s. | n.s. | n.s. | 2.0 | n.s. |
| LOC338758 | 2.2 | 2.7 | n.s. | n.s. | n.s. |
| LOC339483 | −2.7 | n.s. | n.s. | n.s. | n.s. |
| LOC339692 | n.s. | n.s. | −1.8 | n.s. | n.s. |
| LOC344887 | 2.6 | −3.1 | n.s. | n.s. | n.s. |
| LOC346887 | 2.8 | n.s. | n.s. | n.s. | n.s. |
| LOC348174 | n.s. | n.s. | 4.8 | 7.1 | 6.4 |
| LOC348801 | n.s. | −2.1 | n.s. | n.s. | n.s. |
| LOC374491 | n.s. | n.s. | n.s. | n.s. | 1.9 |
| LOC387647 | 2.1 | n.s. | n.s. | n.s. | n.s. |
| LOC387763 | 13.0 | 6.3 | n.s. | n.s. | n.s. |
| LOC388180 | −3.5 | n.s. | n.s. | n.s. | n.s. |
| LOC388237 | −3.3 | n.s. | n.s. | n.s. | n.s. |
| LOC388388 | 2.0 | 1.8 | n.s. | n.s. | n.s. |
| LOC388480 | n.s. | 1.8 | n.s. | n.s. | n.s. |
| LOC388526 | 2.6 | n.s. | n.s. | n.s. | n.s. |
| LOC388620 | −2.2 | −2.4 | n.s. | n.s. | n.s. |
| LOC388727 | −1.9 | n.s. | n.s. | n.s. | n.s. |
| LOC388890 | n.s. | 4.9 | n.s. | n.s. | n.s. |
| LOC388969 | −1.8 | −2.4 | n.s. | n.s. | n.s. |
| LOC389025 | n.s. | n.s. | n.s. | n.s. | 3.7 |
| LOC389072 | 2.4 | n.s. | n.s. | n.s. | n.s. |
| LOC389102 | n.s. | n.s. | 5.3 | 5.0 | n.s. |
| LOC389129 | −11.6 | n.s. | n.s. | n.s. | n.s. |
| LOC389440 | 7.9 | 3.4 | n.s. | n.s. | n.s. |
| LOC389517 | n.s. | n.s. | n.s. | −2.8 | n.s. |
| LOC389831 | −1.9 | −1.8 | n.s. | n.s. | n.s. |
| LOC389834 | n.s. | −2.1 | n.s. | n.s. | n.s. |
| LOC390533 | −2.2 | n.s. | n.s. | n.s. | n.s. |
| LOC390861 | n.s. | 2.7 | n.s. | n.s. | n.s. |
| LOC391426 | n.s. | 4.8 | n.s. | n.s. | n.s. |
| LOC392271 | n.s. | 1.9 | n.s. | n.s. | n.s. |
| LOC392454 | 2.0 | n.s. | n.s. | n.s. | n.s. |
| LOC399786 | 2.1 | n.s. | n.s. | n.s. | n.s. |
| LOC399818 | −1.9 | n.s. | n.s. | n.s. | n.s. |
| LOC399947 | −4.8 | −2.8 | n.s. | n.s. | n.s. |
| LOC399959 | −2.8 | −2.5 | n.s. | n.s. | n.s. |
| LOC400047 | n.s. | 2.7 | n.s. | n.s. | n.s. |
| LOC400464 | −3.9 | n.s. | n.s. | n.s. | n.s. |
| LOC400581 | 35.8 | 62.1 | n.s. | n.s. | n.s. |
| LOC400642 | −1.9 | n.s. | n.s. | n.s. | n.s. |
| LOC400752 | n.s. | n.s. | 3.9 | n.s. | n.s. |
| LOC401020 | n.s. | −1.8 | n.s. | n.s. | n.s. |
| LOC401022 | n.s. | n.s. | n.s. | 1.9 | n.s. |
| LOC401074 | 2.6 | n.s. | n.s. | n.s. | n.s. |
| LOC401216 | 3.2 | n.s. | n.s. | n.s. | n.s. |
| LOC401317 | 8.3 | n.s. | 4.3 | n.s. | n.s. |
| LOC401384 | −1.8 | n.s. | n.s. | n.s. | n.s. |
| LOC401394 | −3.0 | n.s. | n.s. | n.s. | n.s. |
| LOC401504 | −2.1 | −2.1 | n.s. | n.s. | n.s. |
| LOC401537 | n.s. | −2.2 | n.s. | n.s. | n.s. |
| LOC402778 | 2.0 | n.s. | n.s. | n.s. | n.s. |
| LOC439911 | n.s. | −1.9 | n.s. | n.s. | n.s. |
| LOC439962 | n.s. | 1.8 | n.s. | n.s. | n.s. |
| LOC440061 | n.s. | n.s. | n.s. | n.s. | 10.8 |
| LOC440104 | −2.1 | n.s. | n.s. | n.s. | n.s. |
| LOC440135 | −2.2 | n.s. | n.s. | n.s. | n.s. |
| LOC440248 | −2.4 | n.s. | n.s. | n.s. | n.s. |
| LOC440354 | 2.2 | n.s. | n.s. | n.s. | n.s. |
| LOC440426 | −1.9 | n.s. | n.s. | n.s. | n.s. |
| LOC440434 | −2.0 | n.s. | n.s. | n.s. | n.s. |
| LOC440472 | −2.3 | n.s. | n.s. | n.s. | n.s. |
| LOC440731 | 2.0 | n.s. | n.s. | n.s. | n.s. |
| LOC440836 | n.s. | n.s. | n.s. | −2.9 | −2.9 |
| LOC440853 | n.s. | −2.9 | n.s. | n.s. | n.s. |
| LOC440900 | 3.8 | n.s. | n.s. | n.s. | n.s. |
| LOC440993 | −2.8 | n.s. | n.s. | n.s. | n.s. |
| LOC440995 | −2.4 | n.s. | n.s. | n.s. | n.s. |
| LOC441108 | −3.5 | −5.5 | n.s. | n.s. | n.s. |
| LOC441190 | −3.0 | −2.6 | n.s. | n.s. | n.s. |
| LOC441207 | n.s. | −2.9 | n.s. | n.s. | n.s. |
| LOC441208 | n.s. | n.s. | n.s. | 1.9 | n.s. |
| LOC441455 | n.s. | n.s. | −2.0 | n.s. | n.s. |
| LOC441468 | n.s. | −2.7 | n.s. | n.s. | n.s. |
| LOC441778 | −2.3 | −1.8 | n.s. | n.s. | n.s. |
| LOC442013 | n.s. | n.s. | −1.8 | n.s. | n.s. |
| LOC442240 | 2.0 | n.s. | n.s. | n.s. | n.s. |
| LOC442245 | n.s. | n.s. | −1.9 | −1.8 | n.s. |
| LOC442367 | n.s. | −2.1 | n.s. | n.s. | n.s. |
| LOC442370 | 3.1 | n.s. | n.s. | n.s. | n.s. |
| LOC492311 | −2.7 | −2.1 | n.s. | n.s. | n.s. |
| LOC51152 | n.s. | n.s. | n.s. | −2.2 | n.s. |
| LOC51581 | n.s. | n.s. | n.s. | n.s. | 3.9 |
| LOC541471 | n.s. | 2.2 | n.s. | n.s. | n.s. |
| LOC550643 | n.s. | n.s. | 2.4 | n.s. | n.s. |
| LOC554202 | 4.5 | 2.2 | n.s. | n.s. | n.s. |
| LOC554203 | −3.3 | n.s. | n.s. | n.s. | n.s. |
| LOC56757 | −2.8 | n.s. | n.s. | n.s. | n.s. |
| LOC595101 | n.s. | 3.4 | n.s. | n.s. | n.s. |
| LOC63920 | −1.8 | n.s. | n.s. | n.s. | n.s. |
| LOC641298 | 2.2 | 2.9 | n.s. | n.s. | n.s. |
| LOC641999 | n.s. | n.s. | n.s. | −3.1 | −3.7 |
| LOC642398 | n.s. | −3.8 | n.s. | n.s. | n.s. |
| LOC642580 | n.s. | n.s. | 2.7 | n.s. | n.s. |
| LOC642852 | n.s. | −2.8 | n.s. | n.s. | n.s. |
| LOC643072 | n.s. | −1.9 | n.s. | n.s. | n.s. |
| LOC643517 | n.s. | −2.5 | n.s. | n.s. | n.s. |
| LOC643641 | −1.8 | n.s. | n.s. | n.s. | n.s. |
| LOC643650 | 7.6 | n.s. | n.s. | n.s. | n.s. |
| LOC643668 | n.s. | n.s. | −1.9 | n.s. | −1.8 |
| LOC643837 | 2.1 | n.s. | n.s. | n.s. | n.s. |
| LOC644053 | n.s. | n.s. | n.s. | 2.0 | n.s. |
| LOC644192 | n.s. | −4.1 | n.s. | n.s. | n.s. |
| LOC644215 | n.s. | −2.2 | n.s. | n.s. | n.s. |
| LOC644353 | n.s. | n.s. | n.s. | −2.9 | n.s. |
| LOC645233 | 2.2 | n.s. | n.s. | n.s. | n.s. |
| LOC645238 | n.s. | n.s. | 2.1 | 2.1 | n.s. |
| LOC645431 | 2.0 | n.s. | n.s. | n.s. | n.s. |
| LOC645561 | n.s. | −1.8 | 2.0 | 3.9 | 2.6 |
| LOC645634 | −3.6 | n.s. | n.s. | n.s. | n.s. |
| LOC645676 | 2.3 | n.s. | n.s. | n.s. | n.s. |
| LOC646371 | n.s. | n.s. | n.s. | 2.3 | n.s. |
| LOC646450 | 1.8 | n.s. | n.s. | n.s. | n.s. |
| LOC646561 | 2.4 | n.s. | n.s. | n.s. | n.s. |
| LOC646590 | 2.5 | 2.6 | n.s. | n.s. | n.s. |
| LOC646626 | n.s. | 2.7 | n.s. | n.s. | n.s. |
| LOC646762 | n.s. | −2.0 | n.s. | n.s. | n.s. |
| LOC647087 | 2.0 | n.s. | n.s. | n.s. | n.s. |
| LOC647190 | n.s. | 4.4 | n.s. | n.s. | n.s. |
| LOC647305 | 1.9 | 2.0 | n.s. | n.s. | n.s. |
| LOC647859 | n.s. | −2.2 | n.s. | n.s. | n.s. |
| LOC647946 | 1.9 | n.s. | n.s. | n.s. | n.s. |
| LOC647979 | −2.0 | −2.2 | n.s. | n.s. | n.s. |
| LOC648269 | 1.9 | n.s. | n.s. | n.s. | n.s. |
| LOC648498 | n.s. | n.s. | n.s. | −2.1 | n.s. |
| LOC650766 | n.s. | n.s. | n.s. | n.s. | −2.3 |
| LOC650794 | n.s. | −2.5 | n.s. | n.s. | n.s. |
| LOC652968 | −2.3 | −2.6 | n.s. | n.s. | n.s. |
| LOC653256 | 2.1 | n.s. | n.s. | n.s. | n.s. |
| LOC653562 | 5.1 | n.s. | n.s. | n.s. | n.s. |
| LOC653877 | n.s. | 2.1 | n.s. | n.s. | n.s. |
| LOC654779 | 1.9 | 2.0 | n.s. | n.s. | n.s. |
| LOC727773 | −1.9 | n.s. | n.s. | n.s. | n.s. |
| LOC727820 | n.s. | n.s. | 2.7 | 2.1 | n.s. |
| LOC727893 | −3.0 | −2.0 | n.s. | n.s. | n.s. |
| LOC727942 | −1.9 | −2.6 | n.s. | n.s. | n.s. |
| LOC728198 | −2.0 | −2.0 | n.s. | n.s. | n.s. |
| LOC728264 | n.s. | −3.8 | n.s. | n.s. | n.s. |
| LOC728285 | 14.5 | 14.0 | n.s. | n.s. | n.s. |
| LOC728448 | −3.0 | n.s. | n.s. | n.s. | n.s. |
| LOC728499 | n.s. | n.s. | n.s. | 2.7 | n.s. |
| LOC728555 | −2.6 | −2.3 | n.s. | n.s. | n.s. |
| LOC728661 | −2.6 | −1.9 | n.s. | n.s. | n.s. |
| LOC728730 | −2.6 | n.s. | n.s. | n.s. | n.s. |
| LOC729013 | −1.9 | n.s. | n.s. | n.s. | n.s. |
| LOC729082 | −2.7 | n.s. | n.s. | n.s. | n.s. |
| LOC729124 | n.s. | 3.4 | n.s. | n.s. | n.s. |
| LOC729222 | 2.0 | 2.5 | n.s. | n.s. | n.s. |
| LOC729392 | n.s. | n.s. | n.s. | −2.1 | −2.8 |
| LOC729436 | −1.9 | n.s. | n.s. | n.s. | n.s. |
| LOC729446 | −4.7 | −3.2 | n.s. | n.s. | n.s. |
| LOC729570 | n.s. | −2.0 | n.s. | n.s. | n.s. |
| LOC729678 | n.s. | −2.3 | n.s. | n.s. | n.s. |
| LOC729839 | −2.1 | n.s. | n.s. | n.s. | n.s. |
| LOC730057 | n.s. | n.s. | 2.6 | n.s. | n.s. |
| LOC730101 | −2.0 | −3.7 | n.s. | n.s. | n.s. |
| LOC730102 | −1.9 | n.s. | n.s. | n.s. | n.s. |
| LOC730202 | n.s. | 1.8 | n.s. | n.s. | n.s. |
| LOC730259 | 2.9 | n.s. | n.s. | n.s. | n.s. |
| LOC730421 | −2.0 | n.s. | n.s. | n.s. | n.s. |
| LOC731059 | 3.0 | n.s. | n.s. | n.s. | n.s. |
| LOC731484 | −2.7 | n.s. | n.s. | n.s. | n.s. |
| LOC731848 | 2.1 | n.s. | n.s. | n.s. | n.s. |
| LOC731884 | n.s. | n.s. | n.s. | 2.2 | n.s. |
| LOC90586 | n.s. | n.s. | n.s. | 3.5 | n.s. |
| LOC91137 | −2.5 | n.s. | n.s. | n.s. | n.s. |
| LOC91461 | −21.5 | n.s. | n.s. | n.s. | n.s. |
| LOC92017 | −2.9 | n.s. | n.s. | n.s. | n.s. |
| LOC92482 | −1.9 | −1.9 | n.s. | n.s. | n.s. |
| LOC93349 | n.s. | n.s. | n.s. | −2.1 | n.s. |
| LOH11CR2A | −2.1 | −2.1 | n.s. | n.s. | n.s. |
| LOH3CR2A | n.s. | 1.8 | n.s. | n.s. | n.s. |
| LONP2 | −2.8 | n.s. | n.s. | n.s. | n.s. |
| LOXL4 | −11.2 | n.s. | n.s. | n.s. | n.s. |
| LPIN1 | −1.9 | n.s. | n.s. | n.s. | n.s. |
| LPP | −2.9 | n.s. | n.s. | n.s. | n.s. |
| LPPR2 | n.s. | 1.9 | n.s. | n.s. | n.s. |
| LPXN | n.s. | 2.2 | n.s. | n.s. | n.s. |
| LRAP | −2.9 | n.s. | n.s. | n.s. | n.s. |
| LRBA | −2.0 | n.s. | n.s. | n.s. | n.s. |
| LRCH3 | −1.9 | n.s. | n.s. | n.s. | n.s. |
| LRFN4 | 1.9 | n.s. | n.s. | n.s. | n.s. |
| LRIG1 | −2.2 | n.s. | n.s. | n.s. | n.s. |
| LRIG2 | −2.1 | −2.1 | n.s. | n.s. | n.s. |
| LRIG3 | −5.3 | −2.2 | n.s. | n.s. | n.s. |
| LRIT1 | n.s. | n.s. | n.s. | n.s. | −2.5 |
| LRP1 | n.s. | 2.3 | n.s. | n.s. | n.s. |
| LRP11 | −2.0 | n.s. | n.s. | n.s. | n.s. |
| LRP12 | n.s. | 1.9 | n.s. | n.s. | n.s. |
| LRP5L | n.s. | n.s. | 1.9 | n.s. | n.s. |
| LRP6 | −2.8 | −2.2 | n.s. | n.s. | n.s. |
| LRP8 | 3.1 | 3.0 | n.s. | n.s. | n.s. |
| LRPPRC | −2.1 | n.s. | n.s. | n.s. | n.s. |
| LRRC16 | −2.8 | −2.7 | n.s. | n.s. | n.s. |
| LRRC17 | −4.1 | −3.3 | n.s. | n.s. | n.s. |
| LRRC2 | −3.2 | −1.9 | 1.8 | 2.0 | n.s. |
| LRRC23 | n.s. | −2.2 | n.s. | n.s. | n.s. |
| LRRC27 | −2.1 | −1.9 | n.s. | n.s. | n.s. |
| LRRC28 | −1.9 | n.s. | n.s. | n.s. | n.s. |
| LRRC34 | n.s. | −1.8 | n.s. | n.s. | n.s. |
| LRRC37A | −2.1 | n.s. | n.s. | n.s. | n.s. |
| LRRC37A2 | −2.0 | −2.9 | n.s. | n.s. | n.s. |
| LRRC37B2 | n.s. | −2.0 | n.s. | n.s. | n.s. |
| LRRC44 | n.s. | −2.3 | n.s. | n.s. | n.s. |
| LRRC51 | n.s. | −2.5 | n.s. | n.s. | n.s. |
| LRRC58 | n.s. | −2.4 | n.s. | n.s. | n.s. |
| LRRC61 | n.s. | n.s. | n.s. | n.s. | −2.2 |
| LRRC8A | 1.9 | n.s. | n.s. | n.s. | n.s. |
| LRRC8C | n.s. | 2.0 | n.s. | n.s. | n.s. |
| LRRC8E | 1.9 | n.s. | n.s. | n.s. | n.s. |
| LRRFIP1 | −1.8 | 2.8 | n.s. | n.s. | n.s. |
| LRRFIP2 | 3.4 | 2.0 | n.s. | n.s. | n.s. |
| LRRIQ2 | n.s. | n.s. | n.s. | n.s. | 2.0 |
| LRRK1 | −3.1 | n.s. | n.s. | n.s. | n.s. |
| LRRK2 | −14.6 | n.s. | n.s. | n.s. | n.s. |
| LRWD1 | 2.1 | n.s. | n.s. | n.s. | n.s. |
| LSR | 2.7 | n.s. | n.s. | n.s. | n.s. |
| LSS | −2.0 | n.s. | n.s. | n.s. | n.s. |
| LTA4H | −1.9 | n.s. | n.s. | n.s. | n.s. |
| LTB4DH | 2.1 | n.s. | n.s. | n.s. | n.s. |
| LTB4R | n.s. | n.s. | n.s. | −1.8 | n.s. |
| LTB4R2 | n.s. | n.s. | −1.9 | n.s. | −1.8 |
| LTBP4 | −3.2 | n.s. | n.s. | n.s. | n.s. |
| LUM | −2.4 | n.s. | n.s. | n.s. | n.s. |
| LY6E | −1.9 | n.s. | n.s. | n.s. | n.s. |
| LY6G5C | n.s. | −1.9 | n.s. | n.s. | n.s. |
| LY6K | 2.0 | n.s. | n.s. | n.s. | n.s. |
| LYAR | 2.7 | n.s. | n.s. | n.s. | n.s. |
| LYN | 2.0 | n.s. | n.s. | n.s. | n.s. |
| LYPD1 | 4.4 | n.s. | n.s. | n.s. | n.s. |
| LYPD3 | 29.3 | n.s. | n.s. | n.s. | n.s. |
| LYPD6 | −6.6 | n.s. | n.s. | n.s. | n.s. |
| LYPLA2 | n.s. | n.s. | n.s. | −2.2 | n.s. |
| LYPLA3 | 2.3 | n.s. | n.s. | n.s. | n.s. |
| LYPLAL1 | −2.1 | −2.0 | n.s. | n.s. | n.s. |
| LYRM7 | −1.8 | −2.0 | n.s. | n.s. | n.s. |
| LYSMD3 | −1.9 | n.s. | n.s. | n.s. | n.s. |
| LYSMD4 | n.s. | n.s. | 2.0 | n.s. | n.s. |
| LYST | n.s. | −2.1 | n.s. | n.s. | n.s. |
| LZTFL1 | −1.9 | n.s. | n.s. | n.s. | n.s. |
| M74720 | n.s. | n.s. | −1.9 | −1.9 | n.s. |
| MAB21L1 | −2.5 | −2.4 | n.s. | n.s. | n.s. |
| MACF1 | n.s. | 2.4 | n.s. | n.s. | n.s. |
| MAD2L1 | 2.9 | n.s. | n.s. | n.s. | n.s. |
| MADCAM1 | n.s. | n.s. | n.s. | n.s. | 3.1 |
| MAFB | 6.1 | n.s. | n.s. | n.s. | n.s. |
| MAFF | 2.7 | 6.4 | n.s. | n.s. | n.s. |
| MAFG | 4.9 | n.s. | n.s. | n.s. | n.s. |
| MAG1 | 7.3 | n.s. | n.s. | n.s. | n.s. |
| MAGED2 | −1.9 | n.s. | n.s. | n.s. | n.s. |
| MAGED4 | −1.9 | n.s. | n.s. | n.s. | n.s. |
| MAGI1 | −3.5 | n.s. | −2.1 | n.s. | n.s. |
| MAGI2 | n.s. | n.s. | 1.8 | n.s. | n.s. |
| MAGIX | n.s. | n.s. | n.s. | −6.0 | n.s. |
| Magmas | 2.0 | n.s. | n.s. | n.s. | n.s. |
| MAK3 | n.s. | 1.9 | n.s. | n.s. | n.s. |
| MALL | 9.9 | 2.6 | −2.2 | −2.2 | n.s. |
| MALT1 | n.s. | 2.2 | n.s. | n.s. | n.s. |
| MAML2 | −2.6 | n.s. | n.s. | n.s. | n.s. |
| MAML3 | −2.2 | n.s. | n.s. | n.s. | n.s. |
| MAN1A2 | −2.5 | n.s. | n.s. | n.s. | n.s. |
| MAN1B1 | n.s. | n.s. | −1.9 | n.s. | n.s. |
| MAN1C1 | −2.7 | −2.2 | n.s. | −2.0 | n.s. |
| MAN2A1 | −2.3 | n.s. | n.s. | n.s. | n.s. |
| MAP1LC3C | n.s. | n.s. | n.s. | −2.0 | n.s. |
| MAP2K3 | 1.9 | 2.9 | n.s. | n.s. | n.s. |
| MAP2K5 | −2.1 | n.s. | n.s. | n.s. | n.s. |
| MAP2K6 | −13.3 | −5.3 | n.s. | n.s. | n.s. |
| MAP2K7 | n.s. | n.s. | −2.7 | n.s. | n.s. |
| MAP3K1 | −2.0 | n.s. | n.s. | n.s. | n.s. |
| MAP3K2 | n.s. | −2.0 | n.s. | n.s. | n.s. |
| MAP3K4 | 5.4 | 3.4 | n.s. | n.s. | n.s. |
| MAP3K5 | n.s. | 2.2 | n.s. | n.s. | n.s. |
| MAP3K7 | −2.5 | n.s. | n.s. | n.s. | n.s. |
| MAP4K3 | −2.2 | n.s. | n.s. | 1.8 | n.s. |
| MAP4K4 | −2.3 | 4.5 | n.s. | −1.8 | n.s. |
| MAP6 | −2.0 | n.s. | n.s. | n.s. | n.s. |
| MAP7D1 | n.s. | n.s. | −1.8 | n.s. | n.s. |
| MAP7D3 | −3.0 | n.s. | n.s. | n.s. | n.s. |
| MAP9 | −2.5 | −2.7 | n.s. | n.s. | n.s. |
| MAPK12 | n.s. | n.s. | −2.1 | n.s. | n.s. |
| MAPK13 | n.s. | n.s. | −1.8 | n.s. | n.s. |
| MAPK8 | n.s. | 1.9 | n.s. | n.s. | n.s. |
| MAPKBP1 | n.s. | −2.0 | n.s. | n.s. | n.s. |
| MARCKS | −2.9 | n.s. | n.s. | n.s. | n.s. |
| MARCKSL1 | 5.1 | n.s. | n.s. | n.s. | n.s. |
| MARS | −4.5 | n.s. | n.s. | n.s. | n.s. |
| MASP1 | −2.8 | n.s. | n.s. | n.s. | n.s. |
| MASP2 | −2.0 | −2.8 | n.s. | n.s. | n.s. |
| MAST4 | n.s. | −2.2 | n.s. | n.s. | n.s. |
| MASTL | 2.6 | 1.9 | n.s. | n.s. | n.s. |
| MATN2 | −1.9 | n.s. | n.s. | n.s. | n.s. |
| MATR3 | −1.9 | n.s. | n.s. | n.s. | n.s. |
| MBD3 | n.s. | n.s. | −1.9 | n.s. | n.s. |
| MBD5 | −9.5 | −2.4 | n.s. | n.s. | n.s. |
| MBNL1 | −1.9 | 2.4 | n.s. | n.s. | n.s. |
| MBNL2 | 3.3 | n.s. | n.s. | n.s. | n.s. |
| MBOAT2 | −2.6 | n.s. | n.s. | n.s. | n.s. |
| MBP | −1.9 | n.s. | n.s. | n.s. | n.s. |
| MC5R | 5.6 | n.s. | n.s. | n.s. | n.s. |
| MCAM | 4.6 | 2.4 | n.s. | n.s. | n.s. |
| MCART1 | 2.1 | −1.8 | n.s. | n.s. | n.s. |
| MCART6 | −2.7 | −4.0 | n.s. | n.s. | n.s. |
| MCC | −3.4 | n.s. | n.s. | n.s. | n.s. |
| MCM10 | 5.7 | 5.6 | n.s. | n.s. | n.s. |
| MCM2 | 3.2 | 2.5 | n.s. | n.s. | n.s. |
| MCM3 | 1.9 | 1.9 | n.s. | n.s. | n.s. |
| MCM3APAS | −1.8 | n.s. | n.s. | n.s. | n.s. |
| MCM4 | 4.3 | n.s. | n.s. | n.s. | n.s. |
| MCM5 | 3.4 | 1.9 | n.s. | n.s. | n.s. |
| MCM6 | n.s. | 2.7 | n.s. | n.s. | n.s. |
| MCM7 | 3.8 | 2.0 | n.s. | n.s. | n.s. |
| MCM8 | 2.7 | n.s. | n.s. | n.s. | n.s. |
| MCOLN1 | 2.5 | n.s. | n.s. | n.s. | n.s. |
| MCTP2 | n.s. | n.s. | 1.9 | n.s. | n.s. |
| MDFI | n.s. | n.s. | n.s. | n.s. | −1.9 |
| MDFIC | −1.9 | n.s. | n.s. | n.s. | n.s. |
| MDM2 | 5.3 | n.s. | n.s. | n.s. | n.s. |
| MDM4 | −1.8 | n.s. | n.s. | n.s. | n.s. |
| MDN1 | n.s. | 2.4 | n.s. | n.s. | n.s. |
| ME3 | −2.3 | n.s. | n.s. | n.s. | n.s. |
| MECP2 | −2.5 | −2.2 | n.s. | n.s. | n.s. |
| MED13 | −2.2 | −1.9 | n.s. | n.s. | n.s. |
| MED13L | −2.0 | n.s. | n.s. | n.s. | n.s. |
| MED14 | −2.4 | n.s. | n.s. | n.s. | n.s. |
| MED18 | n.s. | −1.8 | n.s. | n.s. | n.s. |
| MED20 | −2.3 | −2.5 | n.s. | n.s. | n.s. |
| MED26 | 3.0 | n.s. | n.s. | n.s. | n.s. |
| MEF2B | 2.3 | n.s. | n.s. | n.s. | n.s. |
| MEG3 | −2.2 | n.s. | n.s. | n.s. | n.s. |
| MEG8 | 2.2 | n.s. | n.s. | n.s. | n.s. |
| MEGF8 | −2.1 | n.s. | n.s. | n.s. | n.s. |
| MEGF9 | n.s. | −1.8 | n.s. | n.s. | n.s. |
| MEI1 | 2.5 | n.s. | n.s. | n.s. | n.s. |
| MEIS1 | −2.0 | −2.4 | n.s. | n.s. | n.s. |
| MEIS2 | −2.1 | −1.8 | n.s. | n.s. | n.s. |
| MEIS3 | −2.2 | n.s. | n.s. | n.s. | n.s. |
| MEIS3P1 | −1.8 | n.s. | n.s. | n.s. | n.s. |
| MELK | 3.8 | n.s. | n.s. | n.s. | n.s. |
| MEOX2 | −7.6 | −5.4 | n.s. | n.s. | n.s. |
| MESDC1 | 2.2 | n.s. | n.s. | n.s. | n.s. |
| MEST | −2.3 | n.s. | n.s. | n.s. | n.s. |
| MET | 1.9 | n.s. | n.s. | n.s. | n.s. |
| METTL1 | 1.9 | 1.9 | n.s. | n.s. | n.s. |
| METTL3 | −8.9 | n.s. | n.s. | n.s. | n.s. |
| METTL6 | −2.5 | n.s. | n.s. | n.s. | n.s. |
| METTL7A | −3.9 | −7.5 | n.s. | n.s. | n.s. |
| MEX3B | n.s. | −2.7 | n.s. | n.s. | n.s. |
| MEX3D | n.s. | 1.8 | n.s. | n.s. | n.s. |
| MFAP3L | 2.2 | n.s. | n.s. | n.s. | n.s. |
| MFAP4 | −2.2 | n.s. | n.s. | n.s. | n.s. |
| MFSD2 | 3.0 | 3.3 | 4.1 | n.s. | n.s. |
| MFSD7 | 4.2 | 2.0 | n.s. | n.s. | n.s. |
| MGA | n.s. | −3.6 | n.s. | n.s. | n.s. |
| MGC102966 | n.s. | n.s. | −1.9 | n.s. | n.s. |
| MGC11102 | 3.1 | n.s. | n.s. | n.s. | n.s. |
| MGC12916 | 3.6 | n.s. | n.s. | n.s. | n.s. |
| MGC12935 | n.s. | 16.9 | n.s. | n.s. | n.s. |
| MGC12965 | n.s. | −2.3 | n.s. | n.s. | n.s. |
| MGC16121 | −4.0 | −4.0 | n.s. | n.s. | n.s. |
| MGC16169 | −3.6 | n.s. | n.s. | n.s. | n.s. |
| MGC17403 | −3.7 | n.s. | n.s. | n.s. | n.s. |
| MGC21874 | 1.8 | n.s. | n.s. | n.s. | n.s. |
| MGC23270 | n.s. | n.s. | n.s. | −2.2 | n.s. |
| MGC23985 | 18.6 | 5.7 | n.s. | n.s. | n.s. |
| MGC24103 | n.s. | n.s. | 2.0 | n.s. | n.s. |
| MGC29891 | −1.9 | n.s. | n.s. | n.s. | n.s. |
| MGC3260 | −3.0 | −2.2 | n.s. | n.s. | n.s. |
| MGC34034 | n.s. | n.s. | n.s. | n.s. | 35.2 |
| MGC34646 | −1.9 | n.s. | n.s. | n.s. | n.s. |
| MGC39584 | n.s. | n.s. | 3.9 | 4.8 | n.s. |
| MGC39900 | n.s. | −2.1 | n.s. | n.s. | n.s. |
| MGC52110 | −2.1 | −1.8 | n.s. | n.s. | n.s. |
| MGC5370 | n.s. | −2.1 | n.s. | n.s. | n.s. |
| MGC5566 | −5.1 | n.s. | n.s. | n.s. | n.s. |
| MGC87042 | 2.6 | 2.9 | n.s. | n.s. | n.s. |
| MGLL | n.s. | 2.3 | n.s. | n.s. | n.s. |
| MIA3 | −1.9 | n.s. | n.s. | −1.9 | n.s. |
| MIAT | −2.9 | n.s. | n.s. | n.s. | n.s. |
| MIB1 | −2.0 | −1.8 | n.s. | n.s. | n.s. |
| MICAL2 | 2.2 | 2.9 | n.s. | n.s. | n.s. |
| MICALL1 | n.s. | 2.4 | n.s. | n.s. | n.s. |
| MICB | 1.9 | n.s. | n.s. | n.s. | n.s. |
| MID1 | −2.0 | −2.2 | n.s. | n.s. | n.s. |
| MIER1 | n.s. | −2.1 | n.s. | n.s. | n.s. |
| MINA | −1.8 | n.s. | n.s. | n.s. | n.s. |
| MIS12 | 2.4 | n.s. | n.s. | n.s. | n.s. |
| MKI67 | 2.7 | n.s. | n.s. | n.s. | n.s. |
| MKKS | 1.9 | n.s. | n.s. | n.s. | n.s. |
| MKL2 | −2.4 | n.s. | n.s. | −2.4 | n.s. |
| MKLN1 | −2.2 | −2.2 | n.s. | n.s. | n.s. |
| MKX | −6.2 | n.s. | n.s. | n.s. | n.s. |
| MLF1 | n.s. | −1.9 | n.s. | n.s. | n.s. |
| MLF1IP | 4.4 | n.s. | n.s. | n.s. | n.s. |
| MLH3 | −2.0 | n.s. | n.s. | n.s. | n.s. |
| MLL | n.s. | −3.2 | n.s. | n.s. | n.s. |
| MLL5 | n.s. | −2.0 | n.s. | n.s. | n.s. |
| MLLT10 | n.s. | 2.6 | n.s. | n.s. | n.s. |
| MLLT11 | 3.3 | n.s. | n.s. | n.s. | n.s. |
| MLLT3 | −1.9 | −1.9 | n.s. | n.s. | n.s. |
| MLLT6 | −2.5 | n.s. | n.s. | n.s. | n.s. |
| MLPH | 2.6 | 3.3 | n.s. | n.s. | n.s. |
| MMD | 3.3 | 2.0 | n.s. | n.s. | n.s. |
| MME | −2.2 | n.s. | n.s. | n.s. | n.s. |
| MMP1 | 3.1 | n.s. | n.s. | n.s. | n.s. |
| MMP10 | 4.6 | n.s. | n.s. | n.s. | n.s. |
| MMP14 | 2.7 | 2.8 | −2.0 | n.s. | n.s. |
| MMP27 | −4.7 | n.s. | n.s. | n.s. | n.s. |
| MN1 | −2.8 | −2.5 | n.s. | n.s. | n.s. |
| MND1 | 2.3 | n.s. | n.s. | n.s. | n.s. |
| MON1A | 2.1 | n.s. | n.s. | n.s. | n.s. |
| MON1B | −2.5 | −2.5 | n.s. | n.s. | n.s. |
| MOSC2 | −2.1 | n.s. | n.s. | n.s. | n.s. |
| MOSPD2 | −2.0 | n.s. | n.s. | n.s. | n.s. |
| MOSPD3 | −1.9 | n.s. | n.s. | n.s. | n.s. |
| MOXD1 | −1.9 | n.s. | n.s. | n.s. | n.s. |
| MPDZ | −2.5 | n.s. | n.s. | n.s. | n.s. |
| MPHOSPH1 | n.s. | −1.8 | n.s. | n.s. | n.s. |
| MPHOSPH9 | n.s. | −1.8 | n.s. | n.s. | n.s. |
| MPP4 | 2.4 | 2.6 | n.s. | n.s. | n.s. |
| MPPED2 | n.s. | n.s. | n.s. | n.s. | 13.9 |
| MPZL1 | 3.2 | n.s. | n.s. | n.s. | n.s. |
| MRAS | 2.9 | 2.0 | n.s. | n.s. | n.s. |
| MRCL3 | 3.8 | 1.8 | n.s. | n.s. | n.s. |
| MREG | n.s. | 2.0 | n.s. | n.s. | n.s. |
| MRPL14 | 2.0 | n.s. | n.s. | n.s. | n.s. |
| MRPL45 | −2.1 | n.s. | n.s. | n.s. | n.s. |
| MRPL52 | 2.1 | n.s. | n.s. | n.s. | n.s. |
| MRPS10 | 1.8 | n.s. | n.s. | n.s. | n.s. |
| MRPS11 | 2.5 | n.s. | n.s. | n.s. | n.s. |
| MRPS12 | 3.2 | n.s. | n.s. | n.s. | n.s. |
| MRPS14 | −2.4 | −1.8 | n.s. | n.s. | n.s. |
| MRPS25 | 2.7 | 2.0 | n.s. | n.s. | n.s. |
| MRPS30 | −1.9 | n.s. | n.s. | n.s. | n.s. |
| MRTO4 | 2.3 | 1.9 | n.s. | n.s. | n.s. |
| MSH4 | n.s. | n.s. | −4.7 | n.s. | n.s. |
| MSI2 | −1.8 | −1.9 | n.s. | n.s. | n.s. |
| MSN | n.s. | 2.2 | n.s. | n.s. | n.s. |
| MSR1 | n.s. | n.s. | n.s. | n.s. | 3.8 |
| MST150 | 2.0 | n.s. | n.s. | n.s. | n.s. |
| MSTO1 | 2.7 | n.s. | n.s. | n.s. | n.s. |
| MSTP9 | −4.0 | n.s. | n.s. | n.s. | n.s. |
| MT1A | 1.9 | n.s. | n.s. | n.s. | n.s. |
| MT1F | 2.2 | n.s. | n.s. | n.s. | n.s. |
| MT1G | 2.6 | n.s. | n.s. | n.s. | n.s. |
| MT1H | 1.8 | n.s. | n.s. | n.s. | n.s. |
| MT1JP | 1.9 | n.s. | n.s. | n.s. | n.s. |
| MT1M | 2.2 | n.s. | n.s. | n.s. | n.s. |
| MT1X | 3.5 | n.s. | n.s. | n.s. | n.s. |
| MTA3 | −2.3 | n.s. | n.s. | n.s. | n.s. |
| MTAP | n.s. | 2.8 | n.s. | n.s. | n.s. |
| MTBP | n.s. | 2.4 | n.s. | n.s. | n.s. |
| MTCH1 | n.s. | n.s. | −1.9 | n.s. | n.s. |
| MTDH | n.s. | −1.9 | n.s. | n.s. | n.s. |
| MTF2 | 2.0 | n.s. | n.s. | n.s. | n.s. |
| MTHFD1 | −2.5 | n.s. | n.s. | n.s. | n.s. |
| MTHFD1L | n.s. | n.s. | n.s. | −6.6 | n.s. |
| MTHFD2L | 3.1 | n.s. | n.s. | n.s. | n.s. |
| MTHFR | n.s. | −2.5 | n.s. | n.s. | n.s. |
| MTL5 | n.s. | n.s. | n.s. | 4.5 | n.s. |
| MTMR11 | −2.0 | n.s. | n.s. | n.s. | n.s. |
| MTMR3 | n.s. | 2.1 | n.s. | n.s. | n.s. |
| MTP18 | 3.3 | 2.2 | n.s. | n.s. | n.s. |
| MTRF1 | 2.1 | n.s. | n.s. | n.s. | n.s. |
| MTRR | n.s. | 2.1 | n.s. | n.s. | n.s. |
| MTSS1 | 2.7 | 2.8 | n.s. | n.s. | n.s. |
| MTX3 | −2.1 | n.s. | n.s. | n.s. | n.s. |
| MUC12 | n.s. | −2.5 | n.s. | n.s. | n.s. |
| MUM1 | −3.2 | −2.7 | n.s. | −1.8 | n.s. |
| MUPCDH | n.s. | n.s. | n.s. | 2.1 | n.s. |
| MVK | n.s. | n.s. | −1.9 | n.s. | n.s. |
| MX2 | −2.7 | n.s. | n.s. | n.s. | n.s. |
| MXD1 | 3.9 | n.s. | n.s. | n.s. | n.s. |
| MXD4 | n.s. | −1.8 | n.s. | n.s. | n.s. |
| MXRA5 | −2.6 | n.s. | n.s. | n.s. | n.s. |
| MYADM | n.s. | 1.9 | n.s. | n.s. | n.s. |
| MYBBP1A | 2.6 | 3.2 | n.s. | n.s. | n.s. |
| MYBL2 | 3.9 | 2.4 | n.s. | n.s. | n.s. |
| MYC | n.s. | 2.0 | n.s. | n.s. | n.s. |
| MYCBP2 | −2.0 | n.s. | n.s. | n.s. | n.s. |
| MYCL1 | n.s. | n.s. | −7.5 | n.s. | n.s. |
| MYEOV | n.s. | n.s. | n.s. | 1.9 | n.s. |
| MYH10 | −1.8 | n.s. | n.s. | n.s. | n.s. |
| MYH11 | −3.8 | −8.3 | n.s. | n.s. | n.s. |
| MYH8 | n.s. | n.s. | n.s. | 2.5 | n.s. |
| MYLIP | 5.4 | n.s. | n.s. | n.s. | n.s. |
| MYLK | −2.1 | n.s. | n.s. | n.s. | n.s. |
| MYNN | 3.9 | 2.9 | n.s. | n.s. | n.s. |
| MYO10 | 15.3 | 3.6 | n.s. | n.s. | n.s. |
| MYO15B | n.s. | n.s. | n.s. | n.s. | −1.9 |
| MYO19 | 2.5 | n.s. | n.s. | n.s. | n.s. |
| MYO1B | −2.0 | n.s. | n.s. | n.s. | n.s. |
| MYO1D | −2.2 | n.s. | n.s. | n.s. | n.s. |
| MYO1E | 1.8 | 2.4 | n.s. | n.s. | n.s. |
| MYO9B | n.s. | 2.0 | n.s. | n.s. | n.s. |
| MYST3 | n.s. | −2.4 | n.s. | n.s. | n.s. |
| MYST4 | −2.1 | −2.0 | n.s. | n.s. | n.s. |
| N4BP2 | −2.2 | n.s. | n.s. | 2.2 | n.s. |
| NAALADL1 | −1.8 | −2.6 | n.s. | n.s. | n.s. |
| NAB2 | n.s. | 1.9 | n.s. | n.s. | n.s. |
| NACA | n.s. | n.s. | 2.3 | 2.0 | 1.9 |
| NADK | 2.3 | n.s. | n.s. | n.s. | n.s. |
| NAG13 | 2.1 | n.s. | n.s. | n.s. | n.s. |
| NAGS | 2.3 | n.s. | n.s. | n.s. | n.s. |
| NAIP | −2.1 | n.s. | n.s. | n.s. | n.s. |
| NANOS1 | n.s. | n.s. | 2.0 | n.s. | n.s. |
| NAP1L4 | n.s. | 2.8 | n.s. | n.s. | n.s. |
| NAPB | n.s. | −2.0 | n.s. | n.s. | n.s. |
| NAPE-PLD | −2.3 | n.s. | n.s. | n.s. | n.s. |
| NAT10 | n.s. | 1.8 | n.s. | n.s. | n.s. |
| NAV1 | −2.3 | n.s. | n.s. | n.s. | n.s. |
| NAV2 | n.s. | 2.4 | n.s. | n.s. | n.s. |
| NAV3 | n.s. | 2.1 | n.s. | n.s. | n.s. |
| NBEA | −4.4 | −2.5 | n.s. | n.s. | n.s. |
| NBL1 | n.s. | n.s. | −1.8 | n.s. | n.s. |
| NBLA00301 | −3.0 | −2.6 | n.s. | n.s. | n.s. |
| NBPF1 | −2.5 | −1.8 | n.s. | n.s. | n.s. |
| NBPF10 | 2.7 | −1.9 | −2.5 | n.s. | n.s. |
| NBPF3 | −1.8 | n.s. | n.s. | n.s. | n.s. |
| NBR2 | −2.1 | n.s. | n.s. | n.s. | n.s. |
| NCALD | −2.1 | n.s. | n.s. | n.s. | n.s. |
| NCAPD3 | 1.9 | 2.1 | n.s. | n.s. | n.s. |
| NCAPG | 4.2 | n.s. | n.s. | n.s. | n.s. |
| NCAPG2 | 2.1 | n.s. | n.s. | n.s. | n.s. |
| NCAPH | 5.3 | n.s. | n.s. | n.s. | n.s. |
| NCAPH2 | 2.4 | n.s. | n.s. | n.s. | n.s. |
| NCF2 | 6.5 | n.s. | n.s. | n.s. | n.s. |
| NCOA1 | n.s. | −2.4 | n.s. | n.s. | n.s. |
| NCOA3 | −2.3 | −1.9 | n.s. | n.s. | n.s. |
| NCR2 | −5.2 | n.s. | n.s. | n.s. | n.s. |
| NDC80 | 3.9 | n.s. | n.s. | n.s. | n.s. |
| NDE1 | n.s. | −1.9 | −2.0 | n.s. | n.s. |
| NDFIP2 | −1.9 | n.s. | n.s. | n.s. | n.s. |
| NDP | −4.7 | n.s. | n.s. | n.s. | n.s. |
| NDUFA5 | −3.1 | n.s. | n.s. | n.s. | n.s. |
| NDUFB2 | n.s. | −2.2 | n.s. | n.s. | n.s. |
| NDUFC1 | n.s. | −4.9 | n.s. | n.s. | n.s. |
| NEDD4L | 3.9 | n.s. | n.s. | n.s. | n.s. |
| NEDD9 | −2.3 | 2.5 | n.s. | n.s. | n.s. |
| NEFM | 9.0 | 2.0 | n.s. | n.s. | n.s. |
| NEGR1 | −1.9 | n.s. | n.s. | n.s. | n.s. |
| NEK1 | n.s. | −2.0 | n.s. | n.s. | n.s. |
| NEK11 | −1.9 | n.s. | n.s. | n.s. | n.s. |
| NEK2 | 2.4 | −2.4 | n.s. | n.s. | n.s. |
| NEK9 | −2.7 | n.s. | n.s. | n.s. | n.s. |
| NELF | n.s. | n.s. | −1.9 | n.s. | n.s. |
| NET1 | 2.5 | 3.3 | n.s. | n.s. | n.s. |
| NETO2 | 5.9 | 2.7 | n.s. | n.s. | n.s. |
| NEU1 | 2.4 | n.s. | n.s. | n.s. | n.s. |
| NEXN | −1.8 | n.s. | n.s. | n.s. | n.s. |
| NF1 | −2.5 | −1.8 | n.s. | n.s. | n.s. |
| NF2 | 1.9 | n.s. | n.s. | n.s. | n.s. |
| NFAT5 | −2.0 | n.s. | n.s. | n.s. | n.s. |
| NFATC2 | n.s. | −1.9 | n.s. | −2.6 | n.s. |
| NFATC2IP | 2.9 | n.s. | −1.8 | −1.9 | n.s. |
| NFATC3 | n.s. | n.s. | −1.9 | n.s. | n.s. |
| NFATC4 | −1.9 | −1.9 | n.s. | n.s. | n.s. |
| NFE2L2 | 4.4 | n.s. | n.s. | n.s. | n.s. |
| NFIA | −2.5 | n.s. | n.s. | n.s. | n.s. |
| NFIB | −2.3 | n.s. | n.s. | n.s. | n.s. |
| NFIL3 | 5.7 | n.s. | n.s. | n.s. | n.s. |
| NFIX | −2.1 | −1.9 | n.s. | n.s. | n.s. |
| NFKBIA | 3.7 | 2.2 | n.s. | n.s. | n.s. |
| NFKBIB | 4.2 | n.s. | −2.1 | n.s. | n.s. |
| NFKBIE | 2.2 | n.s. | n.s. | n.s. | n.s. |
| NFS1 | n.s. | −2.0 | n.s. | n.s. | n.s. |
| NFYA | 2.9 | n.s. | n.s. | n.s. | n.s. |
| NFYB | −1.8 | −1.8 | n.s. | n.s. | n.s. |
| NGLY1 | n.s. | 6.2 | n.s. | n.s. | n.s. |
| NHEDC2 | 2.3 | n.s. | n.s. | n.s. | n.s. |
| NHLRC2 | −2.8 | n.s. | n.s. | n.s. | n.s. |
| NICN1 | −1.9 | n.s. | n.s. | n.s. | n.s. |
| NIN | −1.9 | n.s. | n.s. | n.s. | n.s. |
| NINJ1 | 2.9 | n.s. | n.s. | n.s. | n.s. |
| NIP7 | n.s. | 1.8 | n.s. | n.s. | n.s. |
| NIPA2 | 2.0 | n.s. | n.s. | n.s. | n.s. |
| NIPBL | n.s. | n.s. | n.s. | n.s. | −1.9 |
| NIPSNAP1 | −2.3 | n.s. | −1.8 | n.s. | n.s. |
| NIPSNAP3B | −2.3 | n.s. | n.s. | n.s. | n.s. |
| NISCH | −2.4 | n.s. | n.s. | n.s. | n.s. |
| NIT1 | −3.0 | n.s. | n.s. | n.s. | n.s. |
| NKD2 | 3.4 | n.s. | n.s. | n.s. | n.s. |
| NKPD1 | n.s. | n.s. | −5.5 | n.s. | n.s. |
| NKX3-1 | 6.0 | 5.6 | n.s. | n.s. | n.s. |
| NLGN1 | −2.7 | n.s. | n.s. | n.s. | n.s. |
| NLN | 1.8 | n.s. | n.s. | n.s. | n.s. |
| NLRC5 | n.s. | n.s. | n.s. | n.s. | −1.9 |
| NME1 | 1.9 | n.s. | n.s. | n.s. | n.s. |
| NME5 | −2.7 | n.s. | n.s. | n.s. | n.s. |
| NMNAT2 | 5.4 | 2.2 | n.s. | n.s. | n.s. |
| NMT2 | −2.4 | n.s. | n.s. | n.s. | n.s. |
| NNMT | n.s. | 2.0 | n.s. | n.s. | n.s. |
| NNT | −2.1 | n.s. | n.s. | n.s. | n.s. |
| NOC2L | n.s. | 1.9 | −2.0 | −1.9 | n.s. |
| NOC3L | n.s. | 2.1 | n.s. | n.s. | n.s. |
| NOC4L | 2.0 | n.s. | n.s. | n.s. | n.s. |
| NOG | 3.4 | 3.1 | n.s. | n.s. | n.s. |
| NOL1 | 2.7 | 2.5 | n.s. | n.s. | n.s. |
| NOL12 | 1.9 | −2.3 | n.s. | n.s. | n.s. |
| NOL14 | n.s. | 1.9 | n.s. | n.s. | −1.9 |
| NOL3 | −2.3 | n.s. | n.s. | n.s. | n.s. |
| NOL5A | 2.0 | n.s. | n.s. | n.s. | n.s. |
| NOL6 | n.s. | 2.2 | n.s. | n.s. | n.s. |
| NOLC1 | 2.6 | 2.2 | n.s. | n.s. | n.s. |
| NOPE | −15.5 | −6.7 | n.s. | n.s. | n.s. |
| NOTCH2NL | n.s. | n.s. | n.s. | −2.3 | −2.7 |
| NOV | −2.4 | n.s. | n.s. | n.s. | n.s. |
| NOVA1 | −3.4 | n.s. | n.s. | n.s. | n.s. |
| NOX4 | 2.3 | 2.4 | n.s. | n.s. | n.s. |
| NP | 5.0 | 2.6 | n.s. | n.s. | n.s. |
| N-PAC | −2.3 | n.s. | n.s. | n.s. | n.s. |
| NPAL2 | −2.1 | n.s. | n.s. | n.s. | n.s. |
| NPAL3 | 1.9 | −2.0 | n.s. | n.s. | n.s. |
| NPAS2 | n.s. | 2.7 | n.s. | n.s. | n.s. |
| NPB | n.s. | n.s. | −1.9 | n.s. | −1.9 |
| NPC1 | 2.1 | 2.2 | n.s. | n.s. | n.s. |
| NPEPL1 | −2.5 | n.s. | n.s. | n.s. | n.s. |
| NPEPPS | −1.8 | −2.0 | n.s. | n.s. | n.s. |
| NPFFR1 | n.s. | n.s. | n.s. | n.s. | −2.1 |
| NPHP1 | −1.9 | −2.0 | n.s. | n.s. | n.s. |
| NPHP3 | −1.9 | −1.9 | n.s. | n.s. | n.s. |
| NPL | n.s. | n.s. | 2.3 | n.s. | n.s. |
| NPR3 | 2.8 | 2.7 | n.s. | n.s. | n.s. |
| NPTX2 | 2.5 | n.s. | n.s. | n.s. | n.s. |
| NR1D1 | 5.4 | 13.3 | n.s. | n.s. | n.s. |
| NR1D2 | −2.0 | n.s. | n.s. | n.s. | n.s. |
| NR2C1 | −1.9 | n.s. | n.s. | n.s. | n.s. |
| NR2F2 | −2.1 | −2.0 | n.s. | n.s. | n.s. |
| NR3C1 | −2.7 | −1.9 | n.s. | n.s. | n.s. |
| NR4A1 | 2.8 | 2.9 | n.s. | n.s. | n.s. |
| NR4A2 | 4.5 | 5.6 | n.s. | n.s. | n.s. |
| NR6A1 | n.s. | 4.8 | n.s. | n.s. | n.s. |
| NRBP2 | −2.9 | n.s. | n.s. | n.s. | n.s. |
| NRG1 | 4.3 | n.s. | n.s. | n.s. | n.s. |
| NRIP1 | n.s. | 1.9 | n.s. | n.s. | n.s. |
| NRIP3 | 2.6 | n.s. | n.s. | n.s. | n.s. |
| NRP1 | n.s. | 2.3 | n.s. | n.s. | n.s. |
| NRP2 | 6.0 | 3.2 | n.s. | n.s. | n.s. |
| NSBP1 | n.s. | −3.8 | n.s. | n.s. | n.s. |
| NSMCE4A | −4.5 | n.s. | n.s. | n.s. | n.s. |
| NSUN2 | n.s. | 2.2 | n.s. | n.s. | n.s. |
| NSUN6 | 2.7 | n.s. | n.s. | n.s. | n.s. |
| NT5DC1 | −3.4 | n.s. | n.s. | n.s. | n.s. |
| NT5E | 2.3 | 2.1 | n.s. | n.s. | n.s. |
| NTN4 | −3.4 | n.s. | n.s. | n.s. | n.s. |
| NTRK3 | n.s. | n.s. | n.s. | n.s. | −2.3 |
| NUBPL | −1.9 | n.s. | n.s. | n.s. | n.s. |
| NUDCD1 | n.s. | 2.0 | n.s. | n.s. | 2.1 |
| NUDT10 | n.s. | n.s. | 4.1 | n.s. | n.s. |
| NUDT13 | −1.9 | n.s. | n.s. | n.s. | n.s. |
| NUDT14 | −7.8 | n.s. | n.s. | n.s. | n.s. |
| NUDT15 | 1.8 | n.s. | n.s. | n.s. | n.s. |
| NUDT16 | n.s. | −2.0 | n.s. | n.s. | n.s. |
| NUDT3 | −2.9 | n.s. | n.s. | n.s. | n.s. |
| NUDT6 | −2.3 | −2.1 | n.s. | n.s. | n.s. |
| NUDT7 | −2.2 | n.s. | n.s. | n.s. | n.s. |
| NUF2 | 3.1 | n.s. | n.s. | n.s. | n.s. |
| NUFIP2 | 1.9 | n.s. | n.s. | n.s. | n.s. |
| NUMA1 | −2.4 | n.s. | n.s. | n.s. | n.s. |
| NUMB | n.s. | 1.9 | n.s. | n.s. | n.s. |
| NUP107 | 2.3 | n.s. | n.s. | n.s. | n.s. |
| NUP155 | n.s. | 1.9 | n.s. | n.s. | n.s. |
| NUP35 | 2.0 | n.s. | n.s. | n.s. | n.s. |
| NUP50 | 2.5 | n.s. | n.s. | n.s. | n.s. |
| NUP62 | 2.2 | n.s. | n.s. | n.s. | n.s. |
| NUP85 | 1.9 | n.s. | n.s. | n.s. | n.s. |
| NUP93 | 2.1 | n.s. | n.s. | n.s. | n.s. |
| NUP98 | 1.8 | 1.9 | n.s. | n.s. | n.s. |
| NUPL1 | 2.7 | 2.9 | n.s. | n.s. | n.s. |
| NUSAP1 | 3.2 | −1.9 | n.s. | n.s. | n.s. |
| NXF1 | 2.3 | n.s. | n.s. | n.s. | n.s. |
| NXT1 | 2.4 | 1.9 | n.s. | n.s. | n.s. |
| NY-SAR-48 | 4.8 | n.s. | n.s. | n.s. | n.s. |
| OAZ2 | n.s. | −2.1 | n.s. | n.s. | n.s. |
| OAZ3 | 2.0 | n.s. | n.s. | n.s. | n.s. |
| OBFC2A | n.s. | 2.0 | n.s. | n.s. | n.s. |
| OBSCN | n.s. | n.s. | 2.2 | n.s. | n.s. |
| OBSL1 | −3.0 | n.s. | n.s. | n.s. | n.s. |
| ODC1 | 3.2 | 1.9 | n.s. | n.s. | n.s. |
| ODZ3 | 1.9 | n.s. | n.s. | n.s. | n.s. |
| OGDH | n.s. | 2.5 | n.s. | n.s. | n.s. |
| OGFOD2 | 2.2 | n.s. | n.s. | n.s. | n.s. |
| OGN | −3.6 | n.s. | n.s. | n.s. | n.s. |
| OGT | −2.0 | n.s. | n.s. | n.s. | n.s. |
| OIP5 | 3.2 | −1.9 | n.s. | n.s. | n.s. |
| OLFML1 | −6.7 | n.s. | n.s. | n.s. | n.s. |
| OLFML2A | −8.7 | n.s. | n.s. | n.s. | n.s. |
| OLFML2B | n.s. | −2.3 | n.s. | n.s. | n.s. |
| OMA1 | −2.7 | −2.1 | n.s. | n.s. | n.s. |
| OMD | −18.4 | −2.8 | n.s. | n.s. | n.s. |
| OPA1 | −2.2 | n.s. | n.s. | n.s. | n.s. |
| OPA3 | n.s. | −2.0 | n.s. | n.s. | n.s. |
| OPCML | 3.7 | 1.8 | n.s. | 1.8 | n.s. |
| OPLAH | −2.1 | n.s. | n.s. | n.s. | n.s. |
| OPRD1 | n.s. | n.s. | n.s. | −3.1 | n.s. |
| OPRL1 | n.s. | n.s. | −2.1 | n.s. | n.s. |
| OPTN | −2.6 | n.s. | n.s. | n.s. | n.s. |
| OR10A5 | n.s. | n.s. | n.s. | n.s. | 2.2 |
| ORAI1 | 2.1 | 2.0 | n.s. | n.s. | n.s. |
| ORAOV1 | 2.0 | n.s. | n.s. | n.s. | n.s. |
| ORC1L | n.s. | 4.4 | −2.0 | n.s. | n.s. |
| ORC6L | 3.9 | n.s. | n.s. | n.s. | n.s. |
| ORMDL1 | −1.9 | n.s. | n.s. | n.s. | n.s. |
| OSAP | −7.3 | −2.9 | n.s. | n.s. | n.s. |
| OSBPL10 | n.s. | n.s. | 1.8 | n.s. | n.s. |
| OSBPL1A | −2.1 | −1.8 | n.s. | n.s. | n.s. |
| OSBPL2 | −1.9 | −1.8 | n.s. | n.s. | n.s. |
| OSBPL3 | −3.3 | n.s. | n.s. | n.s. | n.s. |
| OSBPL6 | n.s. | 1.9 | n.s. | n.s. | n.s. |
| OSBPL7 | −1.9 | n.s. | n.s. | n.s. | n.s. |
| OSGIN1 | 9.8 | n.s. | n.s. | n.s. | n.s. |
| OSGIN2 | 2.3 | n.s. | n.s. | n.s. | n.s. |
| OSMR | 2.6 | 2.5 | n.s. | n.s. | n.s. |
| OSR1 | −3.8 | n.s. | n.s. | n.s. | n.s. |
| OSR2 | −2.8 | −3.2 | n.s. | n.s. | n.s. |
| OSTM1 | 2.3 | n.s. | n.s. | n.s. | n.s. |
| OTUD6A | n.s. | n.s. | n.s. | n.s. | 3.2 |
| OTUD7A | n.s. | n.s. | 4.3 | n.s. | n.s. |
| OTUD7B | n.s. | n.s. | n.s. | n.s. | −1.9 |
| P18SRP | −2.2 | −2.0 | n.s. | n.s. | n.s. |
| P2RX5 | 2.3 | 2.2 | −2.0 | n.s. | n.s. |
| P2RY4 | n.s. | n.s. | 1.9 | n.s. | n.s. |
| P2RY5 | −2.5 | −3.4 | n.s. | n.s. | n.s. |
| P4HA3 | 2.5 | n.s. | n.s. | n.s. | n.s. |
| PA2G4 | 2.2 | n.s. | n.s. | n.s. | n.s. |
| PABPC1 | 3.7 | 2.1 | n.s. | n.s. | n.s. |
| PABPC5 | −2.3 | n.s. | n.s. | n.s. | n.s. |
| PABPN1 | −2.0 | n.s. | n.s. | n.s. | n.s. |
| PACS2 | −2.0 | n.s. | n.s. | n.s. | n.s. |
| PACSIN1 | n.s. | n.s. | 2.1 | n.s. | n.s. |
| PACSIN3 | n.s. | n.s. | −2.1 | n.s. | n.s. |
| PAF1 | n.s. | n.s. | −2.2 | n.s. | n.s. |
| PAFAH1B1 | 1.9 | n.s. | n.s. | n.s. | n.s. |
| PAFAH1B3 | 2.1 | n.s. | n.s. | n.s. | n.s. |
| PAFAH2 | n.s. | n.s. | −2.0 | n.s. | n.s. |
| PAG1 | −2.3 | n.s. | n.s. | n.s. | n.s. |
| PAK1 | n.s. | n.s. | −2.0 | n.s. | n.s. |
| PALB2 | n.s. | 1.9 | n.s. | n.s. | n.s. |
| PALLD | −1.8 | n.s. | n.s. | n.s. | n.s. |
| PAN3 | −1.9 | n.s. | n.s. | n.s. | n.s. |
| PANK2 | 2.3 | n.s. | n.s. | n.s. | n.s. |
| PAPD1 | n.s. | −2.0 | n.s. | n.s. | n.s. |
| PAPOLA | −1.8 | n.s. | n.s. | n.s. | n.s. |
| PAPOLG | −1.9 | n.s. | n.s. | n.s. | n.s. |
| PAPPA | 2.1 | 5.4 | n.s. | n.s. | n.s. |
| PAPPA2 | −3.0 | −2.1 | n.s. | n.s. | n.s. |
| PAQR3 | 2.0 | n.s. | n.s. | n.s. | n.s. |
| PAQR4 | n.s. | 3.5 | n.s. | n.s. | n.s. |
| PARD3 | −2.2 | n.s. | n.s. | n.s. | n.s. |
| PARD3B | −6.3 | −1.9 | n.s. | n.s. | n.s. |
| PARP1 | n.s. | 1.8 | n.s. | n.s. | n.s. |
| PARP14 | −3.4 | n.s. | n.s. | n.s. | n.s. |
| PARP2 | 2.1 | n.s. | n.s. | n.s. | n.s. |
| PARP4 | 2.7 | n.s. | n.s. | n.s. | n.s. |
| PARP9 | −3.0 | n.s. | n.s. | n.s. | n.s. |
| PATZ1 | −2.1 | n.s. | n.s. | −1.9 | n.s. |
| PAWR | −2.1 | n.s. | n.s. | n.s. | n.s. |
| PAXIP1 | −2.7 | −3.8 | n.s. | n.s. | n.s. |
| PBEF1 | 2.1 | 2.6 | n.s. | n.s. | n.s. |
| PBK | 2.7 | −1.9 | n.s. | n.s. | n.s. |
| PBLD | −1.9 | n.s. | n.s. | n.s. | n.s. |
| PBX1 | −2.8 | −2.1 | n.s. | n.s. | n.s. |
| PBXIP1 | −1.9 | n.s. | n.s. | n.s. | n.s. |
| PCAF | −2.2 | n.s. | n.s. | n.s. | n.s. |
| PCBD1 | −2.0 | n.s. | n.s. | n.s. | n.s. |
| PCDH18 | −2.8 | −1.9 | n.s. | n.s. | n.s. |
| PCDH7 | −3.8 | n.s. | 1.9 | n.s. | n.s. |
| PCDH9 | 8.4 | 3.8 | n.s. | n.s. | n.s. |
| PCDHB16 | n.s. | n.s. | 1.8 | n.s. | n.s. |
| PCF11 | n.s. | −1.8 | n.s. | n.s. | n.s. |
| PCGF5 | −2.1 | n.s. | n.s. | n.s. | n.s. |
| PCM1 | −4.0 | −2.2 | n.s. | n.s. | n.s. |
| PCMTD1 | −2.2 | −2.4 | n.s. | n.s. | n.s. |
| PCMTD2 | −3.5 | n.s. | n.s. | n.s. | n.s. |
| PCNA | 1.8 | n.s. | n.s. | n.s. | n.s. |
| PCNX | −4.4 | n.s. | n.s. | n.s. | n.s. |
| PCNXL2 | −5.5 | −3.7 | n.s. | n.s. | n.s. |
| PCOTH | 2.4 | n.s. | n.s. | n.s. | n.s. |
| PCSK1 | n.s. | n.s. | n.s. | 3.8 | n.s. |
| PCSK9 | n.s. | n.s. | n.s. | n.s. | 2.6 |
| PCTK2 | n.s. | 2.0 | n.s. | n.s. | n.s. |
| PCYOX1 | −2.4 | n.s. | n.s. | n.s. | n.s. |
| PDCD11 | n.s. | 3.6 | n.s. | n.s. | n.s. |
| PDCD1LG2 | 6.1 | 5.5 | n.s. | n.s. | n.s. |
| PDCD4 | −1.8 | −1.9 | n.s. | n.s. | n.s. |
| PDCD6IP | −2.6 | n.s. | n.s. | n.s. | n.s. |
| PDE11A | n.s. | n.s. | 2.1 | 2.1 | n.s. |
| PDE1A | −2.6 | n.s. | n.s. | n.s. | n.s. |
| PDE4A | −2.3 | n.s. | n.s. | n.s. | n.s. |
| PDE4B | 2.1 | 2.0 | n.s. | n.s. | n.s. |
| PDE4DIP | −1.8 | −1.9 | 1.9 | n.s. | n.s. |
| PDE5A | −3.3 | −2.7 | n.s. | −1.8 | n.s. |
| PDE6D | 2.2 | n.s. | n.s. | n.s. | n.s. |
| PDE7B | −2.2 | −2.2 | n.s. | n.s. | n.s. |
| PDGFD | −10.6 | −2.7 | n.s. | n.s. | n.s. |
| PDGFRA | −3.3 | n.s. | n.s. | n.s. | n.s. |
| PDGFRB | −2.0 | n.s. | n.s. | n.s. | n.s. |
| PDGFRL | −51.3 | n.s. | n.s. | n.s. | n.s. |
| PDK2 | −2.2 | −1.9 | n.s. | n.s. | n.s. |
| PDK4 | n.s. | −14.4 | n.s. | n.s. | n.s. |
| PDLIM4 | 2.3 | 2.6 | n.s. | n.s. | n.s. |
| PDLIM5 | n.s. | 4.1 | n.s. | n.s. | n.s. |
| PDLIM7 | 2.7 | 2.0 | n.s. | n.s. | n.s. |
| PDPR | −3.2 | −2.5 | n.s. | n.s. | n.s. |
| PDRG1 | 2.5 | n.s. | n.s. | n.s. | n.s. |
| PDS5B | −2.3 | −2.1 | n.s. | n.s. | n.s. |
| PDSS1 | 2.6 | n.s. | n.s. | n.s. | n.s. |
| PDXK | 2.4 | n.s. | n.s. | n.s. | n.s. |
| PDXP | 2.9 | n.s. | n.s. | n.s. | n.s. |
| PDZRN3 | −2.3 | n.s. | n.s. | n.s. | n.s. |
| PEAR1 | −3.1 | n.s. | n.s. | n.s. | n.s. |
| PECR | −3.6 | n.s. | n.s. | n.s. | n.s. |
| PELI1 | 2.0 | n.s. | n.s. | n.s. | n.s. |
| PELI2 | −2.7 | −3.6 | n.s. | n.s. | n.s. |
| PEO1 | 3.2 | 2.1 | n.s. | n.s. | n.s. |
| PER1 | 4.1 | n.s. | n.s. | n.s. | n.s. |
| PER2 | n.s. | −2.3 | n.s. | n.s. | n.s. |
| PER3 | n.s. | −2.7 | n.s. | n.s. | n.s. |
| PERLD1 | n.s. | n.s. | n.s. | −3.8 | n.s. |
| PEX13 | 1.8 | −5.2 | n.s. | n.s. | n.s. |
| PF4 | 4.1 | n.s. | n.s. | n.s. | n.s. |
| PF4V1 | 2.6 | n.s. | n.s. | n.s. | n.s. |
| PFAAP5 | n.s. | 1.8 | n.s. | n.s. | n.s. |
| PFDN2 | 1.9 | n.s. | n.s. | n.s. | n.s. |
| PFKFB2 | −2.0 | −2.0 | n.s. | n.s. | n.s. |
| PFKFB3 | n.s. | 2.0 | n.s. | n.s. | n.s. |
| PFKP | 2.9 | n.s. | n.s. | n.s. | n.s. |
| PFN2 | −1.9 | n.s. | n.s. | n.s. | n.s. |
| PGA3 | −7.3 | −2.8 | n.s. | n.s. | n.s. |
| PGAM5 | 2.8 | 1.9 | n.s. | n.s. | n.s. |
| PGAP1 | −2.2 | −2.0 | n.s. | 1.9 | n.s. |
| PGBD3 | −2.1 | n.s. | n.s. | n.s. | n.s. |
| PGCP | −2.0 | n.s. | n.s. | n.s. | n.s. |
| PGF | 4.2 | 4.3 | n.s. | n.s. | n.s. |
| PGGT1B | n.s. | n.s. | n.s. | n.s. | 2.1 |
| PGM2L1 | 2.1 | n.s. | n.s. | n.s. | n.s. |
| PGPEP1 | −2.0 | −2.4 | n.s. | n.s. | n.s. |
| PGRMC1 | n.s. | −1.8 | n.s. | n.s. | n.s. |
| PGRMC2 | −2.9 | n.s. | n.s. | n.s. | n.s. |
| PGS1 | n.s. | n.s. | −1.8 | n.s. | n.s. |
| PHACS | −8.5 | n.s. | n.s. | n.s. | n.s. |
| PHACTR2 | −2.1 | −2.4 | n.s. | n.s. | n.s. |
| PHACTR3 | −3.8 | n.s. | n.s. | n.s. | n.s. |
| PHC1 | −2.1 | n.s. | n.s. | n.s. | n.s. |
| PHC2 | n.s. | 1.8 | n.s. | n.s. | n.s. |
| PHC3 | −1.9 | −2.2 | n.s. | n.s. | n.s. |
| PHEX | −4.0 | −1.9 | n.s. | n.s. | n.s. |
| PHF10 | −2.4 | n.s. | n.s. | n.s. | n.s. |
| PHF17 | −2.7 | n.s. | n.s. | n.s. | n.s. |
| PHF19 | 2.0 | −2.2 | n.s. | n.s. | n.s. |
| PHF2 | −1.9 | n.s. | n.s. | n.s. | n.s. |
| PHF20L1 | −2.0 | n.s. | n.s. | n.s. | n.s. |
| PHGDH | −1.9 | n.s. | n.s. | n.s. | n.s. |
| PHIP | n.s. | −1.9 | n.s. | n.s. | n.s. |
| PHKB | −1.9 | n.s. | n.s. | n.s. | n.s. |
| PHKG1 | n.s. | n.s. | −2.1 | n.s. | −1.9 |
| PHLDA1 | n.s. | 1.8 | n.s. | n.s. | n.s. |
| PHLDA2 | 2.2 | 2.5 | n.s. | n.s. | n.s. |
| PHTF2 | n.s. | n.s. | n.s. | 1.8 | n.s. |
| PHYHD1 | −4.2 | 1.9 | n.s. | n.s. | n.s. |
| PIAS2 | −1.9 | n.s. | n.s. | n.s. | n.s. |
| PIAS3 | n.s. | −2.2 | −2.0 | n.s. | n.s. |
| PICALM | n.s. | 4.3 | n.s. | n.s. | n.s. |
| PICK1 | n.s. | n.s. | n.s. | −1.8 | n.s. |
| PID1 | −1.8 | n.s. | n.s. | n.s. | n.s. |
| PIF1 | n.s. | n.s. | n.s. | n.s. | −2.3 |
| PIGB | −1.9 | n.s. | n.s. | n.s. | n.s. |
| PIGG | n.s. | n.s. | −1.8 | n.s. | n.s. |
| PIGH | 2.2 | n.s. | n.s. | n.s. | n.s. |
| PIGK | −2.0 | n.s. | n.s. | n.s. | n.s. |
| PIGL | n.s. | −2.5 | n.s. | n.s. | 1.9 |
| PIGN | −2.1 | n.s. | n.s. | n.s. | n.s. |
| PIGV | −1.9 | n.s. | n.s. | n.s. | n.s. |
| PIGW | 2.1 | n.s. | n.s. | n.s. | n.s. |
| PIH1D2 | n.s. | −1.9 | n.s. | n.s. | n.s. |
| PIK3C2A | −1.9 | −1.9 | n.s. | n.s. | n.s. |
| PIK3CA | −2.0 | n.s. | n.s. | n.s. | n.s. |
| PIK3CD | n.s. | 2.2 | n.s. | n.s. | n.s. |
| PIK3IP1 | n.s. | n.s. | −2.0 | n.s. | n.s. |
| PIK3R1 | −2.9 | −2.1 | n.s. | n.s. | n.s. |
| PIK3R3 | n.s. | −1.9 | n.s. | −2.1 | −2.7 |
| PINX1 | 2.2 | n.s. | n.s. | n.s. | n.s. |
| PIP5K3 | n.s. | −1.9 | n.s. | n.s. | n.s. |
| PIR | n.s. | −3.6 | n.s. | n.s. | n.s. |
| PITPNC1 | 2.4 | 2.3 | n.s. | n.s. | n.s. |
| PITX2 | n.s. | −1.9 | n.s. | n.s. | n.s. |
| PKD2 | n.s. | n.s. | n.s. | n.s. | −2.0 |
| PKIA | 3.9 | 3.1 | n.s. | n.s. | n.s. |
| PKMYT1 | 2.7 | n.s. | n.s. | n.s. | n.s. |
| PKNOX1 | 2.2 | n.s. | n.s. | n.s. | n.s. |
| PKP4 | −2.0 | 4.0 | n.s. | n.s. | n.s. |
| PLA2G12A | n.s. | −2.1 | n.s. | n.s. | n.s. |
| PLA2G3 | n.s. | n.s. | n.s. | n.s. | 3.1 |
| PLA2R1 | −3.8 | n.s. | n.s. | n.s. | n.s. |
| PLAC1 | 3.0 | n.s. | n.s. | n.s. | n.s. |
| PLAC2 | 2.7 | n.s. | n.s. | n.s. | n.s. |
| PLAC7 | 2.4 | n.s. | n.s. | n.s. | n.s. |
| PLAG1 | n.s. | −2.7 | n.s. | n.s. | n.s. |
| PLAT | n.s. | 3.7 | n.s. | n.s. | n.s. |
| PLAU | 3.3 | 7.5 | n.s. | n.s. | n.s. |
| PLAUR | 4.7 | 3.2 | n.s. | n.s. | n.s. |
| PLCB1 | −7.3 | n.s. | n.s. | n.s. | n.s. |
| PLCD3 | −3.9 | n.s. | n.s. | n.s. | n.s. |
| PLCL2 | −3.3 | n.s. | n.s. | n.s. | n.s. |
| PLCXD1 | 3.2 | 2.5 | n.s. | n.s. | n.s. |
| PLD1 | −5.5 | −5.1 | n.s. | n.s. | n.s. |
| PLEC1 | n.s. | 1.8 | n.s. | n.s. | n.s. |
| PLEK2 | 6.3 | 2.3 | n.s. | n.s. | n.s. |
| PLEKHA2 | n.s. | n.s. | n.s. | −1.9 | −1.9 |
| PLEKHA5 | −2.2 | −1.8 | n.s. | n.s. | n.s. |
| PLEKHA8 | −2.2 | n.s. | n.s. | n.s. | n.s. |
| PLEKHA9 | 2.4 | n.s. | n.s. | n.s. | n.s. |
| PLEKHG5 | −5.7 | −3.0 | n.s. | n.s. | n.s. |
| PLEKHH2 | −12.6 | n.s. | n.s. | n.s. | n.s. |
| PLEKHK1 | n.s. | n.s. | n.s. | 2.8 | n.s. |
| PLEKHO1 | 2.3 | n.s. | n.s. | n.s. | n.s. |
| PLGLB2 | −2.0 | n.s. | n.s. | n.s. | n.s. |
| PLK1 | 3.4 | −1.9 | n.s. | n.s. | n.s. |
| PLK3 | n.s. | 2.4 | n.s. | n.s. | n.s. |
| PLK4 | 3.3 | n.s. | n.s. | n.s. | n.s. |
| PLOD2 | 2.8 | 2.2 | n.s. | n.s. | n.s. |
| PLSCR4 | −4.3 | −2.1 | n.s. | n.s. | n.s. |
| PLXNA1 | −2.4 | n.s. | n.s. | n.s. | n.s. |
| PLXNA2 | n.s. | 2.4 | n.s. | n.s. | n.s. |
| PMAIP1 | 4.8 | 3.4 | n.s. | n.s. | n.s. |
| PML | −2.5 | n.s. | n.s. | n.s. | n.s. |
| PMVK | n.s. | n.s. | −1.8 | n.s. | n.s. |
| PNO1 | 2.1 | 2.0 | n.s. | n.s. | n.s. |
| PNPLA4 | n.s. | n.s. | n.s. | n.s. | 1.8 |
| PODXL | 5.5 | 6.6 | n.s. | n.s. | n.s. |
| POGZ | −2.1 | −2.4 | n.s. | n.s. | n.s. |
| POLA1 | n.s. | 1.8 | n.s. | n.s. | n.s. |
| POLA2 | 3.5 | 2.1 | n.s. | n.s. | n.s. |
| POLD1 | 4.5 | 2.9 | n.s. | n.s. | n.s. |
| POLE | n.s. | n.s. | −2.3 | 2.0 | n.s. |
| POLE2 | 3.6 | n.s. | n.s. | n.s. | n.s. |
| POLE3 | 2.5 | n.s. | n.s. | n.s. | n.s. |
| POLH | n.s. | 2.9 | n.s. | n.s. | n.s. |
| POLI | −4.2 | −2.4 | n.s. | n.s. | n.s. |
| POLK | −2.1 | 4.6 | n.s. | n.s. | n.s. |
| POLQ | 2.3 | n.s. | n.s. | n.s. | n.s. |
| POLR2D | 2.3 | n.s. | n.s. | n.s. | n.s. |
| POLR2H | 1.9 | n.s. | n.s. | n.s. | n.s. |
| POLR3H | 1.9 | n.s. | n.s. | n.s. | n.s. |
| POLR3K | 3.1 | n.s. | n.s. | n.s. | n.s. |
| POLRMT | n.s. | 2.1 | n.s. | n.s. | n.s. |
| POP5 | 1.8 | n.s. | n.s. | n.s. | n.s. |
| POP7 | 2.0 | n.s. | n.s. | n.s. | n.s. |
| POPDC3 | 2.3 | n.s. | n.s. | n.s. | n.s. |
| PPA2 | 2.2 | n.s. | n.s. | n.s. | n.s. |
| PPAN | 1.9 | 1.9 | n.s. | n.s. | n.s. |
| PPAP2A | −2.1 | −1.8 | n.s. | n.s. | n.s. |
| PPAP2B | −2.1 | n.s. | n.s. | n.s. | n.s. |
| PPAPDC1A | 7.3 | 2.0 | n.s. | n.s. | n.s. |
| PPAPDC3 | n.s. | −2.1 | n.s. | n.s. | n.s. |
| PPARA | −3.0 | −2.5 | n.s. | n.s. | n.s. |
| PPAT | 2.3 | 2.0 | n.s. | n.s. | n.s. |
| PPCDC | 2.5 | n.s. | n.s. | n.s. | n.s. |
| PPFIBP1 | n.s. | n.s. | n.s. | −1.9 | −2.1 |
| PPFIBP2 | −4.2 | n.s. | n.s. | n.s. | n.s. |
| PPHLN1 | 2.9 | n.s. | n.s. | n.s. | n.s. |
| PPID | −3.2 | n.s. | n.s. | n.s. | n.s. |
| PPIF | 10.2 | 2.8 | n.s. | n.s. | n.s. |
| PPIG | −2.8 | n.s. | n.s. | n.s. | n.s. |
| PPIH | 2.2 | n.s. | n.s. | n.s. | n.s. |
| PPIL1 | 2.3 | n.s. | n.s. | n.s. | n.s. |
| PPIL2 | n.s. | n.s. | −1.9 | n.s. | n.s. |
| PPIL5 | 2.3 | n.s. | n.s. | n.s. | n.s. |
| PPL | −32.1 | −4.6 | n.s. | n.s. | n.s. |
| PPM1A | −2.0 | −1.8 | n.s. | n.s. | n.s. |
| PPM1D | 2.7 | n.s. | n.s. | n.s. | n.s. |
| PPM1G | n.s. | 2.0 | −1.9 | n.s. | n.s. |
| PPM1M | −2.1 | n.s. | n.s. | n.s. | n.s. |
| PPME1 | 1.8 | n.s. | −3.1 | n.s. | n.s. |
| PPOX | n.s. | n.s. | −2.0 | n.s. | n.s. |
| PPP1CB | −1.8 | −2.1 | n.s. | n.s. | n.s. |
| PPP1R10 | 2.7 | n.s. | n.s. | n.s. | n.s. |
| PPP1R13L | n.s. | 3.9 | n.s. | n.s. | n.s. |
| PPP1R14A | 3.1 | n.s. | n.s. | n.s. | n.s. |
| PPP1R14C | 4.9 | 4.4 | n.s. | n.s. | n.s. |
| PPP1R15A | 5.2 | 1.9 | n.s. | n.s. | n.s. |
| PPP1R3C | n.s. | −2.2 | n.s. | n.s. | n.s. |
| PPP2R2D | 2.2 | n.s. | n.s. | n.s. | n.s. |
| PPP2R3A | −2.0 | n.s. | n.s. | n.s. | n.s. |
| PPP2R3B | n.s. | 1.9 | n.s. | n.s. | n.s. |
| PPP2R5E | −2.7 | −1.9 | n.s. | n.s. | n.s. |
| PPP3CA | 2.8 | 2.1 | n.s. | n.s. | n.s. |
| PPP3R1 | 1.8 | 1.9 | n.s. | n.s. | n.s. |
| PPP4R2 | −1.8 | n.s. | n.s. | n.s. | n.s. |
| PPRC1 | n.s. | 2.0 | n.s. | n.s. | n.s. |
| PPTC7 | n.s. | −1.9 | n.s. | n.s. | n.s. |
| PQLC2 | 2.2 | n.s. | −2.1 | n.s. | n.s. |
| PQLC3 | −2.2 | n.s. | n.s. | n.s. | n.s. |
| PRAGMIN | 2.0 | n.s. | n.s. | n.s. | n.s. |
| PRC1 | 2.8 | n.s. | n.s. | n.s. | n.s. |
| PRDM1 | n.s. | −2.7 | n.s. | n.s. | n.s. |
| PRDM2 | −2.0 | n.s. | n.s. | n.s. | n.s. |
| PRDX3 | n.s. | n.s. | 2.0 | 1.9 | n.s. |
| PRELID1 | 2.2 | n.s. | n.s. | n.s. | n.s. |
| PREPL | −2.1 | n.s. | n.s. | n.s. | n.s. |
| PRICKLE1 | −2.5 | n.s. | n.s. | n.s. | n.s. |
| PRIM1 | 2.1 | n.s. | n.s. | n.s. | n.s. |
| PRKAA1 | −2.7 | n.s. | n.s. | n.s. | n.s. |
| PRKAR1A | −1.8 | n.s. | n.s. | n.s. | n.s. |
| PRKCA | −2.1 | n.s. | n.s. | n.s. | n.s. |
| PRKCE | −2.1 | −2.2 | n.s. | n.s. | n.s. |
| PRKCSH | −1.9 | n.s. | n.s. | n.s. | n.s. |
| PRKD1 | n.s. | 1.8 | n.s. | n.s. | n.s. |
| PRKD3 | −2.0 | n.s. | n.s. | n.s. | n.s. |
| PRKG1 | −3.4 | n.s. | n.s. | n.s. | n.s. |
| PRKRA | −2.7 | −2.3 | n.s. | n.s. | n.s. |
| PRLR | n.s. | 3.2 | n.s. | n.s. | n.s. |
| PRMT2 | −2.2 | n.s. | n.s. | n.s. | n.s. |
| PRO0149 | 1.8 | n.s. | n.s. | n.s. | n.s. |
| PRO1051 | n.s. | n.s. | n.s. | −1.8 | n.s. |
| PRO1146 | 3.1 | n.s. | n.s. | n.s. | n.s. |
| PRO1843 | −2.1 | −2.1 | n.s. | n.s. | n.s. |
| PRO2158 | n.s. | 2.4 | n.s. | n.s. | n.s. |
| PRO2221 | 2.7 | 2.0 | n.s. | n.s. | n.s. |
| PRO2550 | −3.4 | n.s. | n.s. | n.s. | n.s. |
| PRO3098 | n.s. | −2.2 | n.s. | n.s. | n.s. |
| PRO3121 | −2.4 | n.s. | n.s. | n.s. | n.s. |
| PROS1 | −2.5 | n.s. | n.s. | n.s. | n.s. |
| PRPF4 | 2.3 | n.s. | n.s. | n.s. | n.s. |
| PRPH | n.s. | n.s. | n.s. | n.s. | −3.1 |
| PRPS1 | 4.6 | 1.9 | n.s. | n.s. | n.s. |
| PRR11 | n.s. | −2.5 | n.s. | n.s. | n.s. |
| PRR14 | n.s. | −1.9 | n.s. | n.s. | n.s. |
| PRR15 | n.s. | n.s. | 2.4 | 1.8 | n.s. |
| PRR16 | −1.8 | n.s. | n.s. | n.s. | n.s. |
| PRR3 | −2.2 | n.s. | n.s. | n.s. | n.s. |
| PRR6 | 3.3 | 2.2 | n.s. | n.s. | n.s. |
| PRR7 | n.s. | 2.0 | n.s. | n.s. | n.s. |
| PRRT1 | −3.0 | −2.6 | n.s. | n.s. | n.s. |
| PRRT2 | −4.1 | n.s. | n.s. | n.s. | n.s. |
| PRRX1 | −1.9 | n.s. | n.s. | n.s. | n.s. |
| PRRX2 | 1.9 | n.s. | n.s. | n.s. | n.s. |
| PRSS1 | 6.4 | 2.2 | n.s. | n.s. | n.s. |
| PRSS12 | −3.4 | n.s. | n.s. | n.s. | n.s. |
| PRSS2 | 3.2 | n.s. | n.s. | n.s. | n.s. |
| PRSS23 | −2.3 | n.s. | n.s. | n.s. | n.s. |
| PRSS3 | 7.1 | 2.0 | n.s. | n.s. | n.s. |
| PRTFDC1 | −1.9 | n.s. | n.s. | n.s. | n.s. |
| PRUNE2 | −1.8 | n.s. | n.s. | n.s. | n.s. |
| PS1TP4 | −1.9 | n.s. | n.s. | n.s. | n.s. |
| PSAP | n.s. | n.s. | −2.2 | −1.9 | −1.8 |
| PSD3 | −2.0 | −2.6 | n.s. | n.s. | n.s. |
| PSG1 | 2.1 | n.s. | n.s. | n.s. | n.s. |
| PSG2 | 2.5 | 2.1 | n.s. | n.s. | n.s. |
| PSG4 | 2.4 | n.s. | n.s. | n.s. | n.s. |
| PSG9 | n.s. | n.s. | n.s. | n.s. | 21.7 |
| PSMB7 | −1.8 | n.s. | n.s. | n.s. | n.s. |
| PSMB9 | −3.0 | −1.9 | n.s. | n.s. | n.s. |
| PSMC3 | n.s. | n.s. | −2.0 | n.s. | n.s. |
| PSMC3IP | 5.1 | n.s. | n.s. | n.s. | n.s. |
| PSMD10 | n.s. | −1.8 | n.s. | n.s. | n.s. |
| PSMD12 | 1.9 | n.s. | n.s. | n.s. | n.s. |
| PSMD2 | 2.3 | n.s. | −2.1 | n.s. | n.s. |
| PSMD3 | 2.0 | n.s. | n.s. | n.s. | n.s. |
| PSME3 | 4.4 | 1.8 | n.s. | n.s. | n.s. |
| PSME4 | 3.4 | 3.9 | n.s. | n.s. | n.s. |
| PSPH | −2.5 | n.s. | n.s. | n.s. | n.s. |
| PSRC1 | 2.1 | n.s. | n.s. | n.s. | n.s. |
| PTAR1 | n.s. | −2.3 | n.s. | n.s. | n.s. |
| PTBP1 | n.s. | 2.1 | n.s. | n.s. | n.s. |
| PTCD1 | n.s. | 2.1 | n.s. | n.s. | n.s. |
| PTCD3 | −1.9 | n.s. | n.s. | n.s. | n.s. |
| PTDSS2 | n.s. | 2.0 | n.s. | n.s. | n.s. |
| PTEN | −1.9 | n.s. | n.s. | n.s. | n.s. |
| PTGER2 | n.s. | 2.6 | n.s. | n.s. | n.s. |
| PTGER3 | 3.3 | n.s. | n.s. | n.s. | n.s. |
| PTGES | 4.6 | 2.3 | n.s. | n.s. | n.s. |
| PTGFR | −2.1 | −1.8 | n.s. | n.s. | n.s. |
| PTGFRN | −1.9 | n.s. | n.s. | n.s. | n.s. |
| PTGIR | 2.0 | 3.4 | n.s. | n.s. | n.s. |
| PTGIS | −2.7 | −2.2 | n.s. | n.s. | n.s. |
| PTGS2 | n.s. | 7.3 | n.s. | n.s. | n.s. |
| PTHLH | 17.7 | 11.7 | n.s. | n.s. | n.s. |
| PTP4A1 | 2.3 | n.s. | n.s. | n.s. | n.s. |
| PTPLAD2 | −3.0 | n.s. | n.s. | n.s. | n.s. |
| PTPN11 | n.s. | n.s. | n.s. | n.s. | 2.2 |
| PTPN13 | −2.8 | n.s. | n.s. | n.s. | n.s. |
| PTPN21 | −2.5 | n.s. | n.s. | n.s. | n.s. |
| PTPN22 | 4.0 | 4.7 | n.s. | n.s. | n.s. |
| PTPRB | n.s. | 2.6 | n.s. | n.s. | n.s. |
| PTPRE | −2.0 | n.s. | n.s. | n.s. | n.s. |
| PTPRF | −1.8 | 2.1 | n.s. | n.s. | n.s. |
| PTPRM | −1.9 | n.s. | n.s. | n.s. | n.s. |
| PTPRO | 5.0 | 3.7 | n.s. | n.s. | n.s. |
| PTPRR | 5.7 | 3.5 | n.s. | n.s. | n.s. |
| PTPRS | −2.3 | n.s. | n.s. | n.s. | n.s. |
| PTPRU | n.s. | n.s. | −1.9 | n.s. | n.s. |
| PTRH2 | 1.8 | n.s. | n.s. | n.s. | n.s. |
| PTTG1 | 2.4 | −1.9 | n.s. | n.s. | n.s. |
| PTTG3 | 2.4 | n.s. | n.s. | n.s. | n.s. |
| PTX3 | −15.3 | n.s. | n.s. | n.s. | n.s. |
| PUS1 | 1.8 | 3.1 | n.s. | n.s. | n.s. |
| PUS7 | n.s. | 2.3 | n.s. | n.s. | n.s. |
| PUSL1 | 2.0 | n.s. | n.s. | n.s. | n.s. |
| PVR | 3.8 | 5.0 | −2.0 | n.s. | n.s. |
| PVRL2 | 1.9 | n.s. | n.s. | n.s. | n.s. |
| PWP2 | n.s. | 2.1 | n.s. | n.s. | n.s. |
| PXMP3 | −2.0 | −1.8 | n.s. | n.s. | n.s. |
| PXMP4 | n.s. | −1.9 | n.s. | n.s. | n.s. |
| PXN | n.s. | 1.8 | n.s. | n.s. | n.s. |
| PYCARD | −4.5 | 3.6 | n.s. | n.s. | n.s. |
| PYGL | −3.2 | n.s. | n.s. | n.s. | n.s. |
| PYGM | 3.0 | n.s. | n.s. | n.s. | n.s. |
| PYROXD1 | n.s. | n.s. | 1.9 | 2.0 | n.s. |
| QKI | −2.0 | n.s. | n.s. | n.s. | n.s. |
| QPRT | −2.5 | n.s. | n.s. | n.s. | n.s. |
| QSOX1 | −4.1 | n.s. | n.s. | n.s. | n.s. |
| QSOX2 | n.s. | 2.8 | n.s. | n.s. | n.s. |
| R3HDM2 | −1.9 | n.s. | n.s. | n.s. | n.s. |
| RAB11A | −2.0 | n.s. | n.s. | n.s. | n.s. |
| RAB18 | n.s. | 2.1 | n.s. | n.s. | n.s. |
| RAB23 | −2.4 | n.s. | n.s. | n.s. | n.s. |
| RAB26 | −2.2 | n.s. | n.s. | n.s. | n.s. |
| RAB27A | 2.0 | n.s. | n.s. | n.s. | n.s. |
| RAB27B | 2.5 | n.s. | n.s. | n.s. | n.s. |
| RAB30 | n.s. | −2.4 | n.s. | n.s. | n.s. |
| RAB35 | 2.1 | n.s. | n.s. | n.s. | n.s. |
| RAB38 | −15.4 | −4.0 | n.s. | n.s. | n.s. |
| RAB3D | −2.5 | n.s. | n.s. | n.s. | n.s. |
| RAB3GAP2 | −1.9 | −1.8 | n.s. | n.s. | n.s. |
| RAB42 | −2.3 | −2.1 | n.s. | n.s. | n.s. |
| RAB7 | 3.5 | n.s. | n.s. | n.s. | n.s. |
| RAB7A | 1.9 | n.s. | n.s. | n.s. | n.s. |
| RAB7B | −3.4 | −2.8 | n.s. | n.s. | n.s. |
| RAB8B | −2.1 | n.s. | n.s. | n.s. | n.s. |
| RAB9B | n.s. | n.s. | n.s. | 2.8 | n.s. |
| RABGAP1 | −2.5 | −2.0 | n.s. | n.s. | n.s. |
| RABGEF1 | n.s. | −3.7 | n.s. | n.s. | n.s. |
| RABIF | 2.0 | n.s. | n.s. | n.s. | n.s. |
| RACGAP1 | 2.4 | n.s. | n.s. | n.s. | n.s. |
| RAD18 | 1.9 | 2.1 | n.s. | n.s. | n.s. |
| RAD21 | n.s. | −2.7 | n.s. | n.s. | n.s. |
| RAD23B | −1.9 | n.s. | n.s. | n.s. | n.s. |
| RAD51 | 3.5 | n.s. | n.s. | n.s. | n.s. |
| RAD51AP1 | 3.5 | n.s. | n.s. | n.s. | n.s. |
| RAD51L3 | 1.9 | n.s. | n.s. | n.s. | n.s. |
| RAD52 | −1.9 | n.s. | n.s. | n.s. | n.s. |
| RAD54B | 2.9 | n.s. | n.s. | n.s. | n.s. |
| RAD54L | 4.3 | 2.0 | n.s. | n.s. | n.s. |
| RAD9A | −3.5 | n.s. | n.s. | n.s. | n.s. |
| RAGE | 3.9 | n.s. | n.s. | n.s. | n.s. |
| RAI1 | n.s. | n.s. | n.s. | −1.9 | n.s. |
| RALA | 2.4 | n.s. | n.s. | n.s. | n.s. |
| RALBP1 | n.s. | n.s. | n.s. | −1.9 | −2.3 |
| RALGPS2 | −2.1 | n.s. | n.s. | n.s. | n.s. |
| RANBP1 | 1.9 | n.s. | n.s. | n.s. | n.s. |
| RANBP9 | n.s. | 4.4 | n.s. | n.s. | n.s. |
| RANGAP1 | 3.6 | n.s. | −1.9 | n.s. | n.s. |
| RAP2B | −2.1 | −2.3 | n.s. | n.s. | n.s. |
| RAP2C | n.s. | −1.9 | n.s. | n.s. | n.s. |
| RAPGEF1 | n.s. | n.s. | −2.1 | n.s. | n.s. |
| RAPGEF6 | −2.0 | −2.2 | n.s. | n.s. | n.s. |
| RAPH1 | −1.8 | n.s. | n.s. | n.s. | n.s. |
| RARA | −1.8 | n.s. | n.s. | n.s. | n.s. |
| RASA3 | n.s. | 2.9 | n.s. | n.s. | n.s. |
| RASAL2 | n.s. | 3.3 | n.s. | n.s. | n.s. |
| RASD1 | 15.3 | n.s. | n.s. | n.s. | n.s. |
| RASD2 | 7.3 | 4.0 | n.s. | n.s. | n.s. |
| RASEF | 2.3 | n.s. | n.s. | n.s. | n.s. |
| RASGEF1A | −3.9 | n.s. | n.s. | n.s. | n.s. |
| RASL10B | n.s. | n.s. | n.s. | −2.2 | −1.9 |
| RASL11B | 3.1 | n.s. | n.s. | n.s. | n.s. |
| RASSF2 | −14.1 | −6.2 | n.s. | n.s. | n.s. |
| RASSF4 | −2.7 | n.s. | n.s. | n.s. | n.s. |
| RAVER2 | −4.2 | n.s. | n.s. | n.s. | n.s. |
| RB1CC1 | n.s. | −1.9 | n.s. | n.s. | n.s. |
| RBBP6 | 2.2 | n.s. | n.s. | n.s. | n.s. |
| RBCK1 | −2.3 | n.s. | n.s. | n.s. | n.s. |
| RBJ | n.s. | −2.1 | n.s. | n.s. | n.s. |
| RBL2 | −2.1 | n.s. | n.s. | n.s. | n.s. |
| RBM12 | −1.8 | n.s. | n.s. | n.s. | n.s. |
| RBM14 | 2.6 | n.s. | n.s. | n.s. | n.s. |
| RBM17 | −2.0 | n.s. | n.s. | n.s. | n.s. |
| RBM23 | n.s. | n.s. | −2.0 | n.s. | n.s. |
| RBM24 | 2.5 | n.s. | n.s. | n.s. | n.s. |
| RBM30 | −1.9 | −2.0 | n.s. | n.s. | n.s. |
| RBM33 | n.s. | −2.2 | n.s. | n.s. | n.s. |
| RBM35B | n.s. | −1.8 | n.s. | n.s. | n.s. |
| RBM41 | −1.9 | n.s. | n.s. | n.s. | n.s. |
| RBM43 | n.s. | −3.0 | n.s. | n.s. | n.s. |
| RBM4B | n.s. | −2.2 | n.s. | n.s. | n.s. |
| RBM6 | −5.7 | −3.5 | n.s. | n.s. | n.s. |
| RBM7 | −1.8 | n.s. | n.s. | n.s. | n.s. |
| RBM9 | −1.9 | −1.9 | n.s. | n.s. | n.s. |
| RBMS3 | −2.6 | n.s. | n.s. | n.s. | n.s. |
| RBPMS | −1.9 | n.s. | n.s. | n.s. | n.s. |
| RC3H1 | 2.4 | n.s. | n.s. | n.s. | n.s. |
| RC3H2 | 1.8 | n.s. | n.s. | n.s. | n.s. |
| RCAN1 | 3.0 | 3.7 | n.s. | n.s. | n.s. |
| RCAN2 | −44.0 | −4.2 | n.s. | n.s. | n.s. |
| RCL1 | 3.6 | n.s. | n.s. | n.s. | n.s. |
| RCN3 | n.s. | n.s. | −1.8 | n.s. | n.s. |
| RCP9 | n.s. | n.s. | n.s. | n.s. | −2.0 |
| RECK | −3.0 | n.s. | n.s. | n.s. | n.s. |
| RECQL4 | 2.0 | 2.3 | n.s. | n.s. | n.s. |
| REEP3 | −1.8 | n.s. | n.s. | n.s. | n.s. |
| REEP4 | 2.2 | n.s. | n.s. | n.s. | n.s. |
| RELB | n.s. | 2.4 | n.s. | n.s. | n.s. |
| RELT | 24.1 | n.s. | n.s. | n.s. | n.s. |
| RETN | n.s. | n.s. | n.s. | n.s. | −2.8 |
| REV1 | −1.9 | −2.3 | n.s. | n.s. | n.s. |
| REV3L | −4.6 | −3.0 | n.s. | n.s. | n.s. |
| RFC2 | 3.0 | n.s. | n.s. | n.s. | n.s. |
| RFC3 | 5.1 | n.s. | −2.1 | n.s. | n.s. |
| RFC4 | 2.4 | n.s. | n.s. | n.s. | n.s. |
| RFC5 | 2.5 | n.s. | n.s. | n.s. | n.s. |
| RFK | 1.9 | n.s. | n.s. | n.s. | n.s. |
| RFTN1 | −4.0 | n.s. | n.s. | n.s. | n.s. |
| RFTN2 | −2.0 | n.s. | n.s. | n.s. | n.s. |
| RFWD3 | 2.5 | n.s. | n.s. | n.s. | n.s. |
| RFX3 | −2.9 | n.s. | n.s. | n.s. | n.s. |
| RGN | −2.9 | −2.0 | n.s. | n.s. | n.s. |
| RGS12 | n.s. | −2.2 | −1.8 | n.s. | n.s. |
| RGS16 | 3.2 | n.s. | n.s. | n.s. | n.s. |
| RGS17 | −2.2 | n.s. | n.s. | n.s. | n.s. |
| RGS2 | 2.1 | 3.5 | n.s. | n.s. | n.s. |
| RGS20 | 3.9 | 1.9 | n.s. | n.s. | n.s. |
| RGS3 | 2.7 | 3.5 | n.s. | n.s. | n.s. |
| RHBDF2 | n.s. | 2.8 | n.s. | n.s. | n.s. |
| RHEB | n.s. | −2.1 | n.s. | n.s. | n.s. |
| RHEBL1 | 21.9 | n.s. | n.s. | n.s. | n.s. |
| RHOB | n.s. | 2.1 | n.s. | n.s. | n.s. |
| RHOBTB1 | −3.2 | n.s. | n.s. | n.s. | n.s. |
| RHOBTB3 | −1.9 | n.s. | n.s. | n.s. | n.s. |
| RHOJ | −3.1 | n.s. | n.s. | n.s. | n.s. |
| RHOQ | −2.1 | n.s. | n.s. | n.s. | n.s. |
| RICS | −2.1 | n.s. | n.s. | n.s. | n.s. |
| RICTOR | n.s. | −2.7 | n.s. | n.s. | n.s. |
| RIF1 | −1.8 | n.s. | n.s. | −1.8 | n.s. |
| RIG | n.s. | 2.9 | n.s. | n.s. | n.s. |
| RIN2 | −5.1 | n.s. | n.s. | n.s. | n.s. |
| RIN3 | −2.0 | n.s. | n.s. | n.s. | n.s. |
| RIOK3 | 1.9 | 1.9 | n.s. | n.s. | n.s. |
| RIPK3 | −2.0 | n.s. | n.s. | n.s. | n.s. |
| RIPK4 | −2.2 | n.s. | n.s. | n.s. | n.s. |
| RIPK5 | n.s. | −2.0 | n.s. | n.s. | n.s. |
| RIT1 | 1.9 | n.s. | n.s. | n.s. | n.s. |
| RLTPR | 3.7 | n.s. | n.s. | n.s. | n.s. |
| RNASEH2A | 3.1 | n.s. | n.s. | n.s. | n.s. |
| RNASEL | −2.1 | −1.9 | n.s. | n.s. | n.s. |
| RND1 | 7.8 | n.s. | n.s. | n.s. | n.s. |
| RND3 | 2.1 | n.s. | n.s. | n.s. | n.s. |
| RNF111 | n.s. | n.s. | n.s. | n.s. | 1.9 |
| RNF12 | 2.7 | 2.7 | n.s. | n.s. | n.s. |
| RNF122 | 1.9 | n.s. | n.s. | n.s. | n.s. |
| RNF126 | 2.3 | 1.8 | n.s. | n.s. | n.s. |
| RNF128 | 2.6 | n.s. | n.s. | n.s. | n.s. |
| RNF144B | −3.2 | n.s. | n.s. | n.s. | n.s. |
| RNF149 | 2.0 | n.s. | n.s. | n.s. | n.s. |
| RNF150 | −6.6 | −3.1 | n.s. | n.s. | n.s. |
| RNF157 | −6.1 | n.s. | n.s. | n.s. | n.s. |
| RNF170 | −1.8 | n.s. | n.s. | n.s. | n.s. |
| RNF207 | −2.2 | n.s. | n.s. | n.s. | n.s. |
| RNF34 | n.s. | 2.1 | n.s. | n.s. | n.s. |
| RNF5 | 2.2 | n.s. | n.s. | n.s. | n.s. |
| RNMTL1 | 2.0 | n.s. | n.s. | n.s. | n.s. |
| ROBO1 | −2.0 | n.s. | n.s. | n.s. | n.s. |
| ROBO2 | −11.7 | n.s. | n.s. | n.s. | n.s. |
| ROCK2 | −2.6 | −2.1 | n.s. | n.s. | n.s. |
| ROGDI | n.s. | −1.8 | n.s. | n.s. | n.s. |
| ROPN1L | n.s. | n.s. | n.s. | 2.1 | n.s. |
| ROR1 | −5.0 | n.s. | n.s. | n.s. | n.s. |
| ROR2 | n.s. | −7.3 | n.s. | n.s. | n.s. |
| RORA | 2.5 | n.s. | n.s. | n.s. | n.s. |
| RP11- | −2.1 | n.s. | n.s. | n.s. | n.s. |
| 125A7.3 | | | | | |
| RP11-298P3.3 | −1.9 | n.s. | n.s. | n.s. | n.s. |
| RP1-21O18.1 | −2.6 | n.s. | n.s. | n.s. | n.s. |
| RP13- | 3.9 | 3.2 | n.s. | n.s. | n.s. |
| 15M17.2 | | | | | |
| RP3-473B4.1 | n.s. | −3.3 | n.s. | n.s. | n.s. |
| RP5-1022P6.2 | 2.1 | n.s. | n.s. | n.s. | n.s. |
| RP5-886K2.1 | n.s. | 2.4 | n.s. | n.s. | n.s. |
| RPA2 | 2.1 | n.s. | n.s. | n.s. | n.s. |
| RPL13 | 3.8 | 4.1 | n.s. | n.s. | n.s. |
| RPL21 | 1.9 | n.s. | n.s. | n.s. | n.s. |
| RPL31 | −2.2 | n.s. | n.s. | n.s. | n.s. |
| RPL35 | −2.3 | n.s. | n.s. | n.s. | n.s. |
| RPL37A | −2.6 | −4.7 | n.s. | n.s. | n.s. |
| RPL39L | 1.9 | n.s. | n.s. | n.s. | n.s. |
| RPP25 | 2.7 | n.s. | n.s. | n.s. | n.s. |
| RPPH1 | n.s. | 2.0 | n.s. | n.s. | n.s. |
| RPS15A | −2.0 | n.s. | n.s. | n.s. | n.s. |
| RPS24 | −2.0 | n.s. | n.s. | n.s. | n.s. |
| RPS6KA2 | −3.8 | −1.9 | n.s. | n.s. | n.s. |
| RQCD1 | 3.9 | n.s. | n.s. | n.s. | n.s. |
| RRAD | 26.4 | 8.4 | n.s. | n.s. | n.s. |
| RRAGB | −2.1 | n.s. | n.s. | n.s. | n.s. |
| RRAGD | 2.6 | n.s. | n.s. | n.s. | n.s. |
| RRBP1 | n.s. | n.s. | −1.9 | n.s. | n.s. |
| RREB1 | n.s. | −2.3 | n.s. | n.s. | n.s. |
| RRM2 | 5.2 | 2.7 | n.s. | n.s. | n.s. |
| RRN3 | 1.9 | n.s. | n.s. | n.s. | n.s. |
| RRP1 | 2.7 | n.s. | n.s. | n.s. | n.s. |
| RRP12 | 3.2 | 3.1 | n.s. | n.s. | n.s. |
| RRP15 | −2.1 | n.s. | n.s. | n.s. | n.s. |
| RRP9 | 3.2 | 2.0 | n.s. | n.s. | n.s. |
| RRS1 | n.s. | 1.9 | n.s. | n.s. | n.s. |
| RSHL2 | n.s. | −2.6 | n.s. | n.s. | n.s. |
| RSL1D1 | 2.8 | n.s. | n.s. | n.s. | n.s. |
| RSRC1 | −1.9 | n.s. | n.s. | n.s. | n.s. |
| RTN2 | −2.0 | n.s. | n.s. | n.s. | n.s. |
| RTP4 | −5.7 | −2.8 | n.s. | n.s. | n.s. |
| RUNX1 | −2.5 | 3.0 | n.s. | n.s. | n.s. |
| RUNX1T1 | −3.1 | −2.2 | n.s. | n.s. | n.s. |
| RUTBC1 | −2.2 | n.s. | n.s. | n.s. | n.s. |
| RWDD2B | n.s. | −1.9 | n.s. | n.s. | n.s. |
| RXFP3 | n.s. | n.s. | n.s. | 1.8 | n.s. |
| RXRA | 2.0 | n.s. | n.s. | n.s. | n.s. |
| RYBP | 1.9 | n.s. | n.s. | n.s. | n.s. |
| S100A10 | −3.7 | −5.4 | n.s. | n.s. | n.s. |
| S100A2 | 2.2 | n.s. | n.s. | n.s. | n.s. |
| S100A6 | −4.5 | −6.2 | n.s. | n.s. | n.s. |
| S100A7 | 9.4 | n.s. | n.s. | n.s. | n.s. |
| S100P | 3.7 | n.s. | n.s. | n.s. | n.s. |
| SAC3D1 | 2.2 | n.s. | n.s. | n.s. | n.s. |
| SACS | −1.9 | n.s. | n.s. | n.s. | n.s. |
| SAFB2 | n.s. | n.s. | −1.8 | −1.9 | −1.9 |
| SALL1 | 1.9 | n.s. | n.s. | n.s. | n.s. |
| SALL2 | −2.9 | n.s. | n.s. | n.s. | n.s. |
| SAMD1 | 2.4 | 3.1 | n.s. | n.s. | n.s. |
| SAMD11 | 3.1 | 2.7 | n.s. | n.s. | n.s. |
| SAMD4A | −2.0 | n.s. | n.s. | n.s. | n.s. |
| SAMD9L | −1.8 | −1.8 | n.s. | n.s. | n.s. |
| SAMHD1 | −2.2 | n.s. | n.s. | n.s. | n.s. |
| SAP30 | 4.6 | −1.9 | n.s. | n.s. | n.s. |
| SAPS2 | n.s. | 2.0 | n.s. | n.s. | n.s. |
| SARM1 | 2.0 | n.s. | n.s. | n.s. | n.s. |
| SASH1 | −2.0 | −2.4 | n.s. | n.s. | n.s. |
| SAT | n.s. | −2.0 | n.s. | n.s. | n.s. |
| SAT1 | 2.2 | n.s. | n.s. | n.s. | n.s. |
| SATB1 | −2.1 | −2.4 | n.s. | n.s. | n.s. |
| SATB2 | n.s. | 2.2 | n.s. | n.s. | n.s. |
| SATL1 | −2.7 | −2.3 | n.s. | n.s. | n.s. |
| SAV1 | −2.4 | −2.4 | n.s. | n.s. | n.s. |
| SBF2 | −3.1 | n.s. | n.s. | n.s. | n.s. |
| SC4MOL | n.s. | 3.5 | n.s. | n.s. | 2.3 |
| SC5DL | −2.9 | n.s. | n.s. | n.s. | n.s. |
| SCCPDH | −2.0 | n.s. | n.s. | n.s. | n.s. |
| SCD | −2.3 | n.s. | n.s. | n.s. | n.s. |
| SCFV | n.s. | n.s. | n.s. | n.s. | 2.3 |
| SCG5 | 5.9 | 2.8 | n.s. | n.s. | n.s. |
| SCLT1 | 2.8 | n.s. | n.s. | n.s. | n.s. |
| SCLY | 2.1 | n.s. | n.s. | n.s. | n.s. |
| SCRG1 | n.s. | n.s. | 2.0 | n.s. | n.s. |
| SCRN3 | −2.0 | −1.8 | n.s. | n.s. | n.s. |
| SCUBE3 | 3.9 | n.s. | n.s. | n.s. | n.s. |
| SCYE1 | −3.0 | n.s. | n.s. | n.s. | n.s. |
| SDC2 | −1.8 | n.s. | n.s. | n.s. | n.s. |
| SDC3 | −2.3 | n.s. | n.s. | n.s. | n.s. |
| SDCBP2 | 1.9 | n.s. | n.s. | n.s. | n.s. |
| SDCCAG3 | n.s. | 1.9 | n.s. | n.s. | n.s. |
| SDCCAG8 | 2.2 | 4.1 | n.s. | n.s. | n.s. |
| SDF2L1 | 3.6 | n.s. | n.s. | n.s. | n.s. |
| SDHAL2 | n.s. | n.s. | −1.9 | n.s. | n.s. |
| SDHALP2 | −2.0 | −2.1 | n.s. | n.s. | n.s. |
| SDHC | n.s. | n.s. | n.s. | n.s. | −1.8 |
| SDPR | −2.6 | −6.1 | n.s. | n.s. | n.s. |
| SEC11C | 2.2 | n.s. | n.s. | n.s. | n.s. |
| SEC14L1 | 1.9 | n.s. | n.s. | −1.8 | n.s. |
| SEC14L2 | 3.9 | 2.8 | n.s. | n.s. | n.s. |
| SEC22C | 2.1 | n.s. | n.s. | n.s. | n.s. |
| SEC23B | 1.9 | 2.0 | n.s. | n.s. | n.s. |
| SEC24A | n.s. | n.s. | n.s. | 3.1 | 2.0 |
| SEC24C | n.s. | n.s. | −2.1 | n.s. | n.s. |
| SEC31L1 | n.s. | −1.9 | n.s. | n.s. | n.s. |
| SECTM1 | −4.3 | −2.7 | n.s. | n.s. | n.s. |
| SEH1L | n.s. | 1.9 | n.s. | n.s. | n.s. |
| SEL1L | n.s. | −2.4 | n.s. | n.s. | n.s. |
| SELENBP1 | −6.7 | −2.7 | −1.9 | −1.9 | n.s. |
| SELI | 2.5 | n.s. | n.s. | n.s. | n.s. |
| SELO | n.s. | n.s. | −2.1 | n.s. | n.s. |
| SELPLG | 4.8 | 4.2 | n.s. | n.s. | n.s. |
| SEMA3A | −2.2 | n.s. | n.s. | n.s. | n.s. |
| SEMA3B | −4.9 | n.s. | n.s. | n.s. | n.s. |
| SEMA3C | 2.3 | 2.5 | n.s. | n.s. | n.s. |
| SEMA3D | n.s. | −3.1 | n.s. | n.s. | n.s. |
| SEMA4A | n.s. | 5.3 | n.s. | n.s. | n.s. |
| SEMA4C | n.s. | n.s. | −1.9 | n.s. | n.s. |
| SEMA5A | −2.2 | −1.9 | n.s. | n.s. | n.s. |
| SEMA6D | n.s. | n.s. | 2.1 | n.s. | n.s. |
| SENP5 | 2.9 | n.s. | n.s. | n.s. | n.s. |
| SENP6 | −2.1 | n.s. | n.s. | n.s. | n.s. |
| SENP7 | −2.6 | n.s. | n.s. | n.s. | n.s. |
| SENP8 | n.s. | −2.8 | n.s. | n.s. | n.s. |
| SEPHS1 | −2.0 | −1.9 | n.s. | n.s. | n.s. |
| SEPP1 | −2.6 | n.s. | n.s. | n.s. | n.s. |
| SEPSECS | n.s. | n.s. | n.s. | 2.1 | n.s. |
| SERAC1 | −2.7 | −2.2 | n.s. | n.s. | n.s. |
| SERGEF | −2.0 | n.s. | n.s. | n.s. | n.s. |
| SERHL | −1.9 | n.s. | n.s. | n.s. | n.s. |
| SERHL2 | −2.2 | n.s. | n.s. | n.s. | n.s. |
| SERPINB1 | −3.1 | n.s. | n.s. | n.s. | n.s. |
| SERPINB2 | 24.8 | 9.2 | n.s. | n.s. | n.s. |
| SERPINB6 | n.s. | n.s. | −1.8 | n.s. | n.s. |
| SERPINB7 | 2.3 | n.s. | n.s. | n.s. | n.s. |
| SERPINB8 | 3.0 | 1.9 | n.s. | n.s. | n.s. |
| SERPINE1 | 4.9 | 3.4 | n.s. | n.s. | n.s. |
| SERPINE2 | −2.0 | n.s. | n.s. | n.s. | n.s. |
| SERPINF1 | −4.0 | −2.1 | n.s. | n.s. | n.s. |
| SERTAD1 | 3.8 | 2.1 | n.s. | n.s. | n.s. |
| SERTAD4 | −3.6 | −2.3 | n.s. | n.s. | n.s. |
| SESN3 | −2.5 | −3.2 | n.s. | n.s. | n.s. |
| SETBP1 | −2.4 | n.s. | n.s. | n.s. | n.s. |
| SETDB2 | 3.1 | 4.2 | n.s. | n.s. | n.s. |
| SF3B1 | −1.9 | n.s. | n.s. | n.s. | n.s. |
| SF3B2 | n.s. | n.s. | −1.8 | n.s. | n.s. |
| SFN | 3.6 | n.s. | n.s. | n.s. | n.s. |
| SFPQ | −2.3 | n.s. | n.s. | n.s. | n.s. |
| SFRP2 | −3.2 | n.s. | n.s. | n.s. | n.s. |
| SFRP4 | n.s. | n.s. | 1.8 | n.s. | n.s. |
| SFRS1 | −1.8 | n.s. | n.s. | n.s. | n.s. |
| SFRS11 | −2.7 | −1.8 | n.s. | n.s. | n.s. |
| SFRS12 | −2.2 | −2.5 | n.s. | n.s. | n.s. |
| SFRS15 | 2.4 | 1.9 | n.s. | n.s. | n.s. |
| SFRS18 | −2.1 | n.s. | n.s. | n.s. | n.s. |
| SFRS2IP | −2.1 | n.s. | n.s. | n.s. | n.s. |
| SFRS3 | n.s. | 1.9 | n.s. | n.s. | n.s. |
| SFRS5 | 2.4 | 3.5 | n.s. | n.s. | n.s. |
| SFRS6 | n.s. | 2.0 | n.s. | n.s. | n.s. |
| SFRS7 | 1.8 | 1.9 | n.s. | n.s. | n.s. |
| SFXN3 | 2.2 | n.s. | n.s. | n.s. | n.s. |
| SFXN5 | n.s. | n.s. | −2.4 | n.s. | n.s. |
| SGCD | −2.1 | n.s. | n.s. | n.s. | n.s. |
| SGCE | −2.2 | n.s. | n.s. | n.s. | n.s. |
| SGCG | −2.7 | −2.0 | n.s. | n.s. | n.s. |
| SGK | 2.7 | 4.1 | n.s. | n.s. | n.s. |
| SGK269 | −3.0 | n.s. | n.s. | n.s. | n.s. |
| SGMS2 | −4.6 | n.s. | n.s. | n.s. | n.s. |
| SGOL2 | 2.7 | n.s. | n.s. | n.s. | n.s. |
| SGSH | n.s. | 2.2 | n.s. | n.s. | n.s. |
| SGSM2 | −2.0 | n.s. | n.s. | n.s. | n.s. |
| SGTA | 2.1 | n.s. | n.s. | n.s. | n.s. |
| SH2B3 | n.s. | 2.1 | n.s. | n.s. | n.s. |
| SH2D2A | 10.4 | 7.6 | n.s. | n.s. | n.s. |
| SH3BGRL | −2.5 | −2.2 | n.s. | n.s. | n.s. |
| SH3BP2 | 2.4 | n.s. | n.s. | n.s. | n.s. |
| SH3BP4 | 3.9 | n.s. | n.s. | n.s. | n.s. |
| SH3BP5 | n.s. | −3.1 | n.s. | n.s. | n.s. |
| SH3D19 | −1.8 | n.s. | n.s. | n.s. | n.s. |
| SH3GLB1 | −1.9 | n.s. | n.s. | n.s. | n.s. |
| SH3GLB2 | −1.9 | n.s. | n.s. | n.s. | n.s. |
| SH3GLP3 | n.s. | 4.7 | n.s. | n.s. | n.s. |
| SH3MD4 | −4.2 | n.s. | n.s. | n.s. | n.s. |
| SH3RF1 | −1.9 | n.s. | n.s. | n.s. | n.s. |
| SHC2 | n.s. | n.s. | n.s. | n.s. | −3.9 |
| SHC4 | n.s. | n.s. | n.s. | 1.8 | n.s. |
| SHCBP1 | 4.2 | n.s. | n.s. | n.s. | n.s. |
| SHOX2 | −1.8 | −1.8 | n.s. | n.s. | n.s. |
| SHPRH | −4.1 | −3.1 | n.s. | n.s. | n.s. |
| SHROOM3 | −4.5 | −2.3 | n.s. | n.s. | n.s. |
| SIAE | −2.4 | n.s. | n.s. | −1.8 | n.s. |
| SIGIRR | n.s. | −1.9 | n.s. | n.s. | n.s. |
| SIKE | 2.6 | n.s. | n.s. | n.s. | n.s. |
| SIM1 | −3.2 | −2.1 | n.s. | n.s. | n.s. |
| SIMP | 4.4 | n.s. | n.s. | n.s. | n.s. |
| SIPA1L1 | −3.5 | n.s. | n.s. | n.s. | n.s. |
| SIPA1L2 | −3.4 | −3.6 | n.s. | n.s. | n.s. |
| SIPA1L3 | n.s. | 3.8 | n.s. | n.s. | n.s. |
| SIRPA | 2.4 | 2.0 | n.s. | n.s. | n.s. |
| SIRT4 | −2.1 | −3.8 | n.s. | n.s. | n.s. |
| SIRT7 | 2.0 | n.s. | n.s. | n.s. | n.s. |
| SIX4 | −2.2 | n.s. | n.s. | n.s. | n.s. |
| SIX5 | −2.1 | n.s. | n.s. | n.s. | n.s. |
| SKAP2 | −1.8 | −1.8 | n.s. | n.s. | n.s. |
| SKI | n.s. | n.s. | n.s. | −1.9 | −2.2 |
| SKP2 | −2.0 | n.s. | n.s. | n.s. | n.s. |
| SLA/LP | −3.4 | −3.3 | n.s. | n.s. | n.s. |
| SLAIN2 | −2.4 | n.s. | n.s. | n.s. | n.s. |
| SLC12A2 | −2.6 | n.s. | n.s. | n.s. | n.s. |
| SLC12A4 | n.s. | 2.0 | n.s. | n.s. | n.s. |
| SLC14A1 | −6.5 | n.s. | n.s. | n.s. | n.s. |
| SLC15A3 | −3.0 | n.s. | n.s. | n.s. | n.s. |
| SLC15A4 | 1.9 | n.s. | n.s. | n.s. | n.s. |
| SLC16A1 | 3.2 | 2.0 | n.s. | n.s. | n.s. |
| SLC16A12 | n.s. | n.s. | n.s. | 3.9 | n.s. |
| SLC16A14 | n.s. | n.s. | n.s. | n.s. | 2.2 |
| SLC16A3 | 2.2 | 1.8 | n.s. | n.s. | n.s. |
| SLC16A6 | 4.9 | 4.7 | n.s. | n.s. | n.s. |
| SLC16A7 | −3.0 | n.s. | n.s. | n.s. | n.s. |
| SLC19A1 | 3.0 | 3.8 | n.s. | n.s. | n.s. |
| SLC19A2 | 2.9 | 3.1 | n.s. | n.s. | n.s. |
| SLC1A2 | −2.6 | −2.1 | n.s. | n.s. | n.s. |
| SLC1A3 | −2.3 | n.s. | n.s. | n.s. | n.s. |
| SLC1A4 | n.s. | 2.9 | n.s. | n.s. | n.s. |
| SLC20A1 | 2.2 | 4.4 | n.s. | n.s. | n.s. |
| SLC20A2 | 2.0 | 3.3 | n.s. | n.s. | n.s. |
| SLC22A4 | 2.2 | n.s. | n.s. | n.s. | n.s. |
| SLC24A1 | −2.1 | −2.2 | n.s. | n.s. | n.s. |
| SLC25A13 | 3.8 | 2.7 | n.s. | n.s. | n.s. |
| SLC25A22 | 2.2 | 2.1 | n.s. | n.s. | n.s. |
| SLC25A23 | −3.2 | n.s. | n.s. | n.s. | n.s. |
| SLC25A27 | −3.6 | n.s. | n.s. | n.s. | n.s. |
| SLC25A28 | 2.6 | n.s. | n.s. | n.s. | n.s. |
| SLC25A33 | 2.5 | 1.8 | n.s. | n.s. | n.s. |
| SLC25A36 | n.s. | −1.8 | n.s. | n.s. | n.s. |
| SLC25A37 | −2.1 | n.s. | n.s. | n.s. | n.s. |
| SLC25A44 | 1.9 | n.s. | n.s. | n.s. | n.s. |
| SLC27A3 | −2.6 | n.s. | n.s. | n.s. | n.s. |
| SLC27A4 | 7.0 | 4.3 | n.s. | n.s. | n.s. |
| SLC29A1 | 2.0 | n.s. | n.s. | n.s. | n.s. |
| SLC2A1 | 2.4 | n.s. | n.s. | n.s. | n.s. |
| SLC2A10 | −2.3 | n.s. | n.s. | n.s. | n.s. |
| SLC2A14 | n.s. | 2.9 | n.s. | n.s. | n.s. |
| SLC2A3 | n.s. | 3.2 | n.s. | n.s. | n.s. |
| SLC2A6 | n.s. | 2.9 | n.s. | n.s. | n.s. |
| SLC30A1 | 4.6 | 2.1 | n.s. | n.s. | n.s. |
| SLC30A9 | 1.9 | n.s. | n.s. | n.s. | n.s. |
| SLC31A2 | 2.2 | n.s. | n.s. | n.s. | n.s. |
| SLC35A2 | 1.9 | n.s. | n.s. | n.s. | n.s. |
| SLC35A3 | −2.1 | −1.9 | n.s. | n.s. | n.s. |
| SLC35B1 | 1.9 | n.s. | n.s. | n.s. | n.s. |
| SLC35C2 | −2.3 | n.s. | −1.9 | n.s. | n.s. |
| SLC35E2 | −2.5 | n.s. | n.s. | n.s. | n.s. |
| SLC35E4 | 2.0 | n.s. | n.s. | n.s. | n.s. |
| SLC35F2 | 1.8 | 2.3 | n.s. | n.s. | n.s. |
| SLC36A1 | 2.3 | 2.0 | n.s. | n.s. | n.s. |
| SLC38A1 | −1.8 | n.s. | n.s. | n.s. | n.s. |
| SLC38A4 | −3.3 | n.s. | n.s. | n.s. | n.s. |
| SLC38A5 | 2.2 | n.s. | n.s. | n.s. | n.s. |
| SLC39A11 | 2.5 | n.s. | n.s. | n.s. | n.s. |
| SLC3A2 | 3.5 | n.s. | n.s. | n.s. | n.s. |
| SLC43A2 | 2.1 | n.s. | n.s. | n.s. | n.s. |
| SLC44A1 | −2.0 | −2.1 | n.s. | n.s. | n.s. |
| SLC46A3 | −3.5 | n.s. | n.s. | n.s. | n.s. |
| SLC4A2 | n.s. | 1.8 | n.s. | n.s. | n.s. |
| SLC4A4 | 2.3 | 2.5 | n.s. | n.s. | n.s. |
| SLC4A7 | n.s. | 1.8 | n.s. | n.s. | n.s. |
| SLC4A8 | n.s. | −2.3 | n.s. | n.s. | n.s. |
| SLC5A3 | −2.3 | n.s. | n.s. | n.s. | n.s. |
| SLC5A6 | 3.6 | 2.3 | n.s. | n.s. | n.s. |
| SLC6A19 | 8.1 | n.s. | n.s. | n.s. | n.s. |
| SLC6A6 | 17.2 | n.s. | n.s. | n.s. | n.s. |
| SLC6A8 | 3.1 | 2.0 | n.s. | n.s. | n.s. |
| SLC7A1 | n.s. | 3.8 | n.s. | n.s. | n.s. |
| SLC7A11 | 3.4 | −2.4 | n.s. | n.s. | n.s. |
| SLC7A5 | 2.9 | n.s. | n.s. | n.s. | n.s. |
| SLC7A6 | 2.0 | 1.8 | n.s. | n.s. | n.s. |
| SLC8A1 | n.s. | 2.8 | n.s. | n.s. | n.s. |
| SLC9A3 | −2.3 | −2.3 | n.s. | n.s. | n.s. |
| SLC9A5 | −2.9 | n.s. | n.s. | n.s. | n.s. |
| SLC9A9 | −2.9 | n.s. | n.s. | n.s. | n.s. |
| SLFN11 | −2.1 | n.s. | n.s. | n.s. | n.s. |
| SLIT2 | −1.9 | n.s. | n.s. | n.s. | n.s. |
| SLIT3 | −2.1 | n.s. | n.s. | n.s. | n.s. |
| SLK | −2.2 | n.s. | n.s. | n.s. | n.s. |
| SLN | 4.5 | 2.4 | n.s. | n.s. | n.s. |
| SLTM | n.s. | −1.9 | n.s. | n.s. | n.s. |
| SMAD2 | −1.9 | n.s. | n.s. | n.s. | n.s. |
| SMAD3 | −2.3 | −1.8 | n.s. | n.s. | n.s. |
| SMAD4 | −2.3 | n.s. | n.s. | n.s. | n.s. |
| SMAD5 | −1.9 | n.s. | n.s. | n.s. | n.s. |
| SMAD7 | 2.1 | 2.4 | n.s. | n.s. | n.s. |
| SMARCA1 | −2.0 | n.s. | n.s. | n.s. | n.s. |
| SMARCA2 | −1.8 | n.s. | n.s. | n.s. | n.s. |
| SMARCA4 | 2.2 | 2.2 | −2.1 | n.s. | n.s. |
| SMARCB1 | −3.3 | n.s. | n.s. | n.s. | n.s. |
| SMARCC2 | −2.0 | n.s. | n.s. | n.s. | n.s. |
| SMARCD2 | n.s. | n.s. | −1.9 | n.s. | n.s. |
| SMC2 | 2.3 | n.s. | n.s. | n.s. | n.s. |
| SMC4 | 1.8 | −2.0 | −4.5 | n.s. | n.s. |
| SMCHD1 | n.s. | n.s. | 2.4 | n.s. | n.s. |
| SMCR7L | 1.8 | n.s. | n.s. | n.s. | n.s. |
| SMG6 | n.s. | 2.2 | n.s. | n.s. | n.s. |
| SMOX | 2.6 | 2.1 | n.s. | n.s. | n.s. |
| SMPD4 | 2.1 | n.s. | n.s. | n.s. | n.s. |
| SMTN | 2.6 | n.s. | n.s. | n.s. | n.s. |
| SMURF2 | −2.3 | 2.6 | n.s. | n.s. | n.s. |
| SMYD3 | 3.4 | 2.7 | n.s. | n.s. | n.s. |
| SMYD4 | n.s. | n.s. | −1.8 | n.s. | −2.6 |
| SNAG1 | −2.4 | n.s. | n.s. | n.s. | n.s. |
| SNAI1 | 3.5 | n.s. | n.s. | n.s. | n.s. |
| SNAP23 | −2.3 | n.s. | n.s. | n.s. | n.s. |
| SND1 | n.s. | 2.7 | n.s. | n.s. | n.s. |
| SNED1 | −2.6 | n.s. | n.s. | n.s. | n.s. |
| SNF1LK2 | n.s. | −1.8 | n.s. | n.s. | n.s. |
| SNHG10 | −2.3 | −2.1 | n.s. | n.s. | n.s. |
| SNHG5 | 2.5 | 3.1 | n.s. | n.s. | n.s. |
| SNHG7 | 1.9 | 2.0 | n.s. | n.s. | n.s. |
| SNHG9 | 2.8 | n.s. | n.s. | n.s. | n.s. |
| SNORA28 | 3.7 | 3.8 | n.s. | n.s. | n.s. |
| SNORD114-3 | 4.2 | 3.8 | n.s. | n.s. | n.s. |
| SNRP70 | n.s. | n.s. | −2.1 | n.s. | n.s. |
| SNRPA1 | 2.7 | n.s. | n.s. | n.s. | n.s. |
| SNRPG | −2.7 | n.s. | n.s. | n.s. | n.s. |
| SNRPN | n.s. | −2.5 | n.s. | n.s. | n.s. |
| SNTB2 | −2.8 | −2.2 | n.s. | n.s. | n.s. |
| SNX1 | −3.9 | −2.8 | n.s. | n.s. | n.s. |
| SNX11 | 2.6 | n.s. | n.s. | n.s. | n.s. |
| SNX12 | n.s. | n.s. | −1.8 | n.s. | n.s. |
| SNX13 | −2.4 | −2.3 | n.s. | n.s. | n.s. |
| SNX17 | n.s. | n.s. | −2.1 | n.s. | n.s. |
| SNX5 | 3.5 | n.s. | n.s. | n.s. | n.s. |
| SNX7 | −2.0 | n.s. | n.s. | n.s. | n.s. |
| SNX8 | 2.5 | −2.0 | n.s. | n.s. | n.s. |
| SOAT1 | −2.2 | n.s. | n.s. | n.s. | n.s. |
| SOCS1 | n.s. | −2.6 | n.s. | n.s. | n.s. |
| SOCS3 | 2.6 | 5.0 | n.s. | n.s. | n.s. |
| SOCS5 | −2.1 | n.s. | n.s. | n.s. | n.s. |
| SOCS7 | n.s. | −2.6 | n.s. | n.s. | n.s. |
| SOD2 | 5.1 | 2.1 | n.s. | n.s. | n.s. |
| SOLH | n.s. | n.s. | n.s. | n.s. | 3.1 |
| SORBS2 | 2.9 | n.s. | n.s. | n.s. | n.s. |
| SORD | n.s. | n.s. | n.s. | n.s. | −1.8 |
| SOS1 | −1.9 | n.s. | n.s. | n.s. | n.s. |
| SOST | 3.7 | n.s. | n.s. | n.s. | n.s. |
| SOX4 | 2.6 | n.s. | n.s. | n.s. | n.s. |
| SOX9 | 3.9 | 3.7 | n.s. | n.s. | n.s. |
| SP110 | −2.6 | n.s. | n.s. | n.s. | n.s. |
| SPA17 | n.s. | −2.1 | n.s. | n.s. | n.s. |
| SPAG16 | −2.5 | n.s. | n.s. | n.s. | n.s. |
| SPAG5 | 3.6 | n.s. | n.s. | n.s. | n.s. |
| SPANXA2 | 3.1 | n.s. | n.s. | n.s. | n.s. |
| SPAST | −1.8 | n.s. | n.s. | n.s. | n.s. |
| SPATA13 | n.s. | n.s. | n.s. | −2.4 | −1.8 |
| SPATA17 | −2.7 | n.s. | n.s. | n.s. | n.s. |
| SPATA18 | −4.0 | n.s. | n.s. | n.s. | n.s. |
| SPATA20 | −2.5 | n.s. | n.s. | n.s. | n.s. |
| SPATA6 | −3.8 | n.s. | n.s. | n.s. | n.s. |
| SPATA7 | −2.5 | −2.0 | n.s. | n.s. | n.s. |
| SPC24 | 2.7 | n.s. | n.s. | n.s. | n.s. |
| SPC25 | 4.0 | n.s. | n.s. | n.s. | n.s. |
| SPCS3 | 2.1 | n.s. | n.s. | n.s. | n.s. |
| SPEF2 | −2.6 | −1.9 | n.s. | n.s. | n.s. |
| SPG11 | −2.4 | n.s. | n.s. | n.s. | n.s. |
| SPG3A | n.s. | −1.8 | n.s. | n.s. | n.s. |
| SPG7 | −2.0 | n.s. | n.s. | n.s. | n.s. |
| SPHK1 | 3.3 | 4.0 | n.s. | n.s. | n.s. |
| SPIN2B | −2.4 | n.s. | n.s. | n.s. | n.s. |
| SPIN3 | −2.0 | −2.3 | n.s. | n.s. | n.s. |
| SPINK1 | 7.4 | n.s. | n.s. | n.s. | n.s. |
| SPNS1 | 1.8 | n.s. | n.s. | n.s. | n.s. |
| SPOCD1 | 2.4 | n.s. | n.s. | n.s. | n.s. |
| SPP1 | 3.8 | n.s. | n.s. | n.s. | n.s. |
| SPRED1 | −2.1 | n.s. | n.s. | n.s. | n.s. |
| SPRY1 | −2.3 | n.s. | n.s. | n.s. | n.s. |
| SPRY2 | 4.6 | 7.1 | n.s. | n.s. | n.s. |
| SPRY4 | 1.8 | 8.7 | n.s. | n.s. | n.s. |
| SPSB1 | 5.8 | n.s. | n.s. | n.s. | n.s. |
| SPTBN1 | −2.1 | n.s. | n.s. | n.s. | n.s. |
| SPTLC2 | 3.0 | 2.0 | n.s. | n.s. | n.s. |
| SPTY2D1 | 2.1 | n.s. | n.s. | n.s. | n.s. |
| SQLE | −2.9 | n.s. | n.s. | n.s. | n.s. |
| SQSTM1 | 6.1 | n.s. | n.s. | n.s. | n.s. |
| SR140 | −2.2 | n.s. | n.s. | n.s. | n.s. |
| SRGAP2P1 | n.s. | −1.8 | n.s. | n.s. | n.s. |
| SRGN | 2.2 | n.s. | n.s. | n.s. | n.s. |
| SRM | 2.0 | n.s. | n.s. | n.s. | n.s. |
| SRPK2 | 2.2 | 2.6 | n.s. | n.s. | n.s. |
| SRPRB | 2.0 | n.s. | n.s. | n.s. | n.s. |
| SRXN1 | 3.7 | n.s. | n.s. | n.s. | n.s. |
| SS18 | n.s. | −2.2 | n.s. | n.s. | n.s. |
| SSBP2 | −1.8 | −1.9 | n.s. | n.s. | n.s. |
| SSH1 | 3.1 | n.s. | n.s. | n.s. | n.s. |
| SSPN | −3.1 | −2.1 | n.s. | n.s. | n.s. |
| SSR3 | 2.0 | n.s. | n.s. | n.s. | n.s. |
| SSSCA1 | 2.7 | n.s. | n.s. | n.s. | n.s. |
| ST3GAL1 | 1.9 | 2.0 | n.s. | n.s. | n.s. |
| ST6GALNAC2 | n.s. | n.s. | −2.2 | n.s. | n.s. |
| ST6GALNAC5 | −3.6 | −2.1 | n.s. | n.s. | n.s. |
| ST6GALNAC6 | n.s. | n.s. | −2.1 | n.s. | n.s. |
| ST7L | −2.3 | n.s. | n.s. | n.s. | n.s. |
| ST8SIA1 | −15.4 | n.s. | n.s. | n.s. | n.s. |
| STAC | −4.3 | −2.0 | n.s. | n.s. | n.s. |
| STAM2 | −4.0 | −2.2 | n.s. | n.s. | n.s. |
| STAMBPL1 | −2.0 | −2.2 | n.s. | n.s. | n.s. |
| STARD13 | −4.3 | −2.1 | n.s. | n.s. | n.s. |
| STARD3 | n.s. | n.s. | −1.8 | n.s. | −1.9 |
| STARD4 | −2.1 | n.s. | n.s. | n.s. | n.s. |
| STARD5 | n.s. | −2.3 | n.s. | n.s. | n.s. |
| STARD8 | 2.3 | n.s. | n.s. | n.s. | n.s. |
| STAT2 | −2.7 | n.s. | n.s. | n.s. | n.s. |
| STAT3 | n.s. | 1.8 | −1.8 | n.s. | n.s. |
| STAT4 | −4.1 | n.s. | n.s. | n.s. | n.s. |
| STATIP1 | −1.9 | n.s. | n.s. | n.s. | n.s. |
| STC1 | 6.5 | 6.2 | n.s. | n.s. | n.s. |
| STC2 | 3.1 | n.s. | n.s. | n.s. | n.s. |
| STEAP1 | 2.0 | n.s. | n.s. | n.s. | n.s. |
| STIL | 2.1 | n.s. | n.s. | n.s. | n.s. |
| STIP1 | n.s. | n.s. | −1.9 | n.s. | n.s. |
| STK10 | −2.1 | n.s. | n.s. | n.s. | n.s. |
| STK11IP | −2.4 | n.s. | n.s. | n.s. | n.s. |
| STK17A | 2.1 | 1.9 | n.s. | n.s. | n.s. |
| STK17B | 3.0 | n.s. | n.s. | n.s. | n.s. |
| STK32B | −12.9 | n.s. | n.s. | n.s. | n.s. |
| STK32C | n.s. | −2.0 | n.s. | n.s. | n.s. |
| STK36 | −2.1 | n.s. | n.s. | n.s. | n.s. |
| STK38 | −1.9 | n.s. | n.s. | n.s. | n.s. |
| STK4 | n.s. | n.s. | n.s. | n.s. | 2.3 |
| STMN1 | 2.1 | n.s. | n.s. | n.s. | n.s. |
| STOML1 | n.s. | n.s. | −1.9 | n.s. | n.s. |
| STON1 | −3.2 | −2.8 | n.s. | n.s. | n.s. |
| STRA13 | 3.7 | n.s. | n.s. | n.s. | n.s. |
| STRA6 | 3.1 | 2.0 | n.s. | n.s. | n.s. |
| STS-1 | −3.3 | 2.0 | n.s. | n.s. | n.s. |
| STX17 | −1.8 | n.s. | n.s. | n.s. | n.s. |
| STX1A | 2.3 | 2.7 | n.s. | n.s. | n.s. |
| STX3 | 3.9 | n.s. | n.s. | n.s. | n.s. |
| STX6 | 2.6 | n.s. | n.s. | n.s. | n.s. |
| STXBP5 | −2.3 | n.s. | n.s. | n.s. | n.s. |
| STXBP6 | −2.2 | n.s. | n.s. | n.s. | n.s. |
| STYX | n.s. | n.s. | n.s. | 2.8 | n.s. |
| SUDS3 | −2.4 | n.s. | n.s. | n.s. | n.s. |
| SUGT1L1 | −5.6 | n.s. | n.s. | n.s. | n.s. |
| SUOX | n.s. | −1.9 | n.s. | n.s. | n.s. |
| SUPT4H1 | −2.2 | n.s. | n.s. | n.s. | n.s. |
| SURF-4 | 2.1 | n.s. | n.s. | n.s. | n.s. |
| SUV39H1 | 2.9 | n.s. | −2.2 | n.s. | n.s. |
| SUV420H1 | −3.3 | −3.2 | n.s. | n.s. | n.s. |
| SUZ12P | n.s. | n.s. | 2.7 | n.s. | n.s. |
| SVEP1 | n.s. | 2.0 | n.s. | n.s. | n.s. |
| SWAP70 | −2.6 | n.s. | n.s. | n.s. | n.s. |
| SYDE2 | −2.6 | −2.1 | n.s. | n.s. | n.s. |
| SYNCRIP | −2.0 | n.s. | n.s. | n.s. | n.s. |
| SYNE1 | n.s. | 2.8 | n.s. | n.s. | n.s. |
| SYNGR1 | −2.0 | n.s. | n.s. | n.s. | n.s. |
| SYNGR2 | n.s. | n.s. | −1.9 | n.s. | n.s. |
| SYNJ2 | 21.0 | 2.2 | n.s. | n.s. | n.s. |
| SYNJ2BP | −2.0 | −2.9 | n.s. | n.s. | n.s. |
| SYNPO2 | −3.1 | −3.0 | n.s. | n.s. | n.s. |
| SYT11 | −2.2 | −2.5 | n.s. | n.s. | n.s. |
| SYT15 | 3.2 | n.s. | n.s. | n.s. | n.s. |
| SYTL2 | −2.4 | n.s. | n.s. | n.s. | n.s. |
| SYTL3 | 6.9 | 3.6 | n.s. | n.s. | n.s. |
| SYTL4 | −1.9 | n.s. | n.s. | n.s. | n.s. |
| T62549 | n.s. | n.s. | 2.5 | 2.5 | n.s. |
| T70285 | n.s. | n.s. | n.s. | 3.3 | n.s. |
| TAC1 | 2.7 | n.s. | n.s. | n.s. | n.s. |
| TACC3 | 4.9 | n.s. | n.s. | n.s. | n.s. |
| TAF13 | 3.2 | n.s. | n.s. | n.s. | n.s. |
| TAF1B | −2.0 | n.s. | n.s. | n.s. | n.s. |
| TAF3 | n.s. | −1.9 | n.s. | n.s. | n.s. |
| TAF4 | n.s. | n.s. | 1.8 | n.s. | n.s. |
| TAF4B | n.s. | n.s. | n.s. | −2.1 | n.s. |
| TAF6L | −2.9 | n.s. | n.s. | n.s. | n.s. |
| TAGLN | 2.7 | 1.9 | n.s. | n.s. | n.s. |
| TAGLN3 | 2.3 | n.s. | n.s. | n.s. | n.s. |
| TAP1 | n.s. | −2.9 | n.s. | n.s. | n.s. |
| TAP2 | n.s. | 1.8 | n.s. | n.s. | n.s. |
| TAPBPL | −2.1 | n.s. | n.s. | n.s. | n.s. |
| TAS2R44 | n.s. | n.s. | n.s. | n.s. | 1.9 |
| TAT | −2.3 | n.s. | n.s. | n.s. | n.s. |
| TATDN2 | 2.7 | 1.8 | n.s. | n.s. | n.s. |
| TAX1BP1 | −2.1 | −1.8 | n.s. | n.s. | n.s. |
| TBC1D12 | −1.8 | n.s. | n.s. | n.s. | n.s. |
| TBC1D16 | −3.3 | n.s. | n.s. | n.s. | n.s. |
| TBC1D17 | −2.0 | n.s. | n.s. | n.s. | n.s. |
| TBC1D2 | 2.3 | 2.0 | n.s. | n.s. | n.s. |
| TBC1D24 | −2.0 | n.s. | n.s. | n.s. | n.s. |
| TBC1D2B | −2.6 | n.s. | n.s. | n.s. | n.s. |
| TBC1D3F | n.s. | −2.5 | n.s. | n.s. | n.s. |
| TBC1D5 | −2.7 | n.s. | n.s. | n.s. | n.s. |
| TBC1D8 | −2.5 | 2.7 | n.s. | n.s. | n.s. |
| TBC1D8B | −2.3 | n.s. | 2.2 | 2.3 | n.s. |
| TBCA | 2.1 | n.s. | n.s. | n.s. | n.s. |
| TBCD | n.s. | n.s. | −1.8 | n.s. | n.s. |
| TBL1XR1 | −2.1 | −2.2 | n.s. | n.s. | n.s. |
| TBRG1 | 2.1 | n.s. | n.s. | n.s. | n.s. |
| TBX15 | −2.1 | −2.1 | n.s. | n.s. | n.s. |
| TBX18 | n.s. | −1.8 | n.s. | n.s. | n.s. |
| TBX2 | 1.9 | 5.9 | n.s. | n.s. | n.s. |
| TBX3 | 2.5 | n.s. | n.s. | n.s. | n.s. |
| TBX5 | −2.5 | −2.2 | n.s. | n.s. | n.s. |
| TCEA1 | −2.0 | n.s. | n.s. | n.s. | n.s. |
| TCEA2 | −2.3 | n.s. | n.s. | n.s. | n.s. |
| TCEA3 | −2.6 | n.s. | n.s. | n.s. | n.s. |
| TCEB3 | 1.9 | n.s. | n.s. | n.s. | n.s. |
| TCF12 | −2.1 | −1.9 | n.s. | n.s. | n.s. |
| TCF15 | n.s. | n.s. | n.s. | n.s. | −2.2 |
| TCF19 | 3.0 | n.s. | n.s. | n.s. | n.s. |
| TCF25 | n.s. | n.s. | −2.1 | n.s. | n.s. |
| TCF3 | −1.9 | n.s. | n.s. | n.s. | n.s. |
| TCF4 | −2.2 | −2.0 | n.s. | n.s. | n.s. |
| TCF7L1 | −2.2 | n.s. | n.s. | n.s. | n.s. |
| TCF7L2 | −2.4 | −2.1 | n.s. | n.s. | n.s. |
| TCF8 | −2.6 | n.s. | n.s. | n.s. | n.s. |
| TCIRG1 | 2.2 | 1.9 | n.s. | n.s. | n.s. |
| TCTEX1D1 | −5.0 | n.s. | n.s. | n.s. | n.s. |
| TDG | 2.6 | n.s. | n.s. | n.s. | n.s. |
| TDO2 | n.s. | n.s. | n.s. | 3.7 | n.s. |
| TDP1 | 2.5 | n.s. | n.s. | n.s. | n.s. |
| TEAD1 | −2.7 | n.s. | n.s. | n.s. | n.s. |
| TEAD4 | n.s. | 3.1 | n.s. | n.s. | n.s. |
| TEF | n.s. | n.s. | −1.8 | n.s. | n.s. |
| TELO2 | n.s. | 2.0 | n.s. | n.s. | n.s. |
| TENC1 | −2.0 | n.s. | n.s. | n.s. | n.s. |
| TEP1 | −1.9 | n.s. | n.s. | n.s. | n.s. |
| TERF2 | 2.2 | n.s. | n.s. | n.s. | n.s. |
| TES | 1.8 | n.s. | n.s. | n.s. | n.s. |
| TESK1 | 1.9 | n.s. | n.s. | n.s. | n.s. |
| TEX10 | 1.9 | 1.9 | n.s. | n.s. | n.s. |
| TFDP1 | 2.4 | n.s. | n.s. | n.s. | n.s. |
| TFDP2 | −2.0 | −2.5 | n.s. | n.s. | n.s. |
| TFPI2 | 5.6 | n.s. | n.s. | n.s. | n.s. |
| TGFA | 4.7 | 4.5 | n.s. | n.s. | n.s. |
| TGFB1 | 2.0 | n.s. | n.s. | n.s. | n.s. |
| TGFBR3 | −2.7 | n.s. | n.s. | n.s. | n.s. |
| TGM2 | 7.2 | 3.0 | −1.8 | n.s. | n.s. |
| TH | n.s. | n.s. | n.s. | −3.0 | n.s. |
| THADA | −1.8 | 2.2 | n.s. | n.s. | n.s. |
| THAP2 | 2.7 | n.s. | n.s. | n.s. | n.s. |
| THAP5 | n.s. | n.s. | 3.2 | n.s. | 3.3 |
| THBD | 9.7 | 3.1 | n.s. | n.s. | n.s. |
| THBS1 | −1.9 | 2.0 | n.s. | n.s. | n.s. |
| THBS2 | −1.9 | n.s. | n.s. | n.s. | n.s. |
| THC2235542 | n.s. | n.s. | 1.8 | n.s. | 2.3 |
| THC2266906 | n.s. | n.s. | n.s. | 2.0 | 1.8 |
| THC2274697 | n.s. | n.s. | 1.8 | n.s. | n.s. |
| THC2278725 | n.s. | n.s. | n.s. | n.s. | −4.0 |
| THC2279825 | n.s. | n.s. | n.s. | 1.9 | n.s. |
| THC2279910 | n.s. | n.s. | 2.2 | n.s. | 2.1 |
| THC2280343 | n.s. | n.s. | 2.0 | n.s. | n.s. |
| THC2280741 | n.s. | n.s. | n.s. | n.s. | 2.3 |
| THC2281350 | n.s. | n.s. | 2.4 | n.s. | n.s. |
| THC2282972 | n.s. | n.s. | n.s. | −2.2 | n.s. |
| THC2284350 | n.s. | n.s. | 2.1 | n.s. | n.s. |
| THC2290002 | n.s. | n.s. | n.s. | −2.1 | −1.9 |
| THC2308340 | n.s. | n.s. | 1.8 | n.s. | n.s. |
| THC2311764 | n.s. | n.s. | −1.9 | n.s. | n.s. |
| THC2312756 | n.s. | n.s. | 2.8 | n.s. | 4.0 |
| THC2312785 | n.s. | n.s. | 1.9 | n.s. | n.s. |
| THC2312955 | n.s. | n.s. | −1.9 | n.s. | n.s. |
| THC2314215 | n.s. | n.s. | 2.0 | n.s. | n.s. |
| THC2315330 | n.s. | n.s. | 1.9 | n.s. | n.s. |
| THC2316649 | n.s. | n.s. | 3.0 | n.s. | n.s. |
| THC2316768 | n.s. | n.s. | 3.0 | n.s. | 2.2 |
| THC2316936 | n.s. | n.s. | 3.8 | n.s. | n.s. |
| THC2317182 | n.s. | n.s. | n.s. | −4.6 | n.s. |
| THC2319152 | n.s. | n.s. | 11.4 | 7.4 | 10.6 |
| THC2320257 | n.s. | n.s. | 1.8 | n.s. | n.s. |
| THC2322443 | n.s. | n.s. | n.s. | n.s. | 2.5 |
| THC2324430 | n.s. | n.s. | n.s. | n.s. | 2.1 |
| THC2337372 | n.s. | n.s. | 2.5 | n.s. | n.s. |
| THC2337493 | n.s. | n.s. | n.s. | 2.4 | n.s. |
| THC2338537 | n.s. | n.s. | 2.9 | n.s. | n.s. |
| THC2339241 | n.s. | n.s. | 2.3 | n.s. | 1.9 |
| THC2339455 | n.s. | n.s. | 2.5 | n.s. | 3.7 |
| THC2340757 | n.s. | n.s. | n.s. | −1.8 | n.s. |
| THC2342473 | n.s. | n.s. | 3.9 | n.s. | 2.5 |
| THC2343350 | n.s. | n.s. | 1.8 | 1.9 | 2.6 |
| THC2345075 | n.s. | n.s. | 1.9 | n.s. | n.s. |
| THC2356023 | n.s. | n.s. | n.s. | −1.9 | n.s. |
| THC2358845 | n.s. | n.s. | 2.0 | n.s. | n.s. |
| THC2360810 | n.s. | n.s. | n.s. | n.s. | 1.8 |
| THC2360912 | n.s. | n.s. | −1.8 | −1.9 | −1.9 |
| THC2361491 | n.s. | n.s. | n.s. | −2.7 | n.s. |
| THC2361914 | n.s. | n.s. | n.s. | −2.0 | n.s. |
| THC2368209 | n.s. | n.s. | 2.5 | n.s. | 2.5 |
| THC2369020 | n.s. | n.s. | n.s. | n.s. | 4.4 |
| THC2374442 | n.s. | n.s. | −6.8 | n.s. | n.s. |
| THC2375512 | n.s. | n.s. | n.s. | 3.1 | n.s. |
| THC2375853 | n.s. | n.s. | n.s. | n.s. | 2.5 |
| THC2376418 | n.s. | n.s. | 1.8 | n.s. | n.s. |
| THC2376586 | n.s. | n.s. | 2.0 | n.s. | n.s. |
| THC2378378 | n.s. | n.s. | n.s. | n.s. | 3.0 |
| THC2378839 | n.s. | n.s. | 4.5 | n.s. | 4.4 |
| THC2378865 | n.s. | n.s. | 2.0 | n.s. | n.s. |
| THC2378994 | n.s. | n.s. | 4.5 | n.s. | 5.1 |
| THC2381061 | n.s. | n.s. | 1.9 | n.s. | n.s. |
| THC2381707 | n.s. | n.s. | 2.1 | n.s. | n.s. |
| THC2382717 | n.s. | n.s. | n.s. | n.s. | 2.7 |
| THC2397757 | n.s. | n.s. | 2.3 | 2.5 | 2.8 |
| THC2400593 | n.s. | n.s. | n.s. | n.s. | 2.2 |
| THC2404671 | n.s. | n.s. | n.s. | 1.8 | n.s. |
| THC2405319 | n.s. | n.s. | 1.9 | n.s. | n.s. |
| THC2405710 | n.s. | n.s. | 1.8 | n.s. | n.s. |
| THC2405842 | n.s. | n.s. | 1.9 | 2.0 | n.s. |
| THC2405936 | n.s. | n.s. | 2.1 | n.s. | n.s. |
| THC2406017 | n.s. | n.s. | 4.9 | n.s. | n.s. |
| THC2406779 | n.s. | n.s. | n.s. | 3.1 | n.s. |
| THC2406786 | n.s. | n.s. | n.s. | −6.1 | n.s. |
| THC2406944 | n.s. | n.s. | 1.8 | n.s. | n.s. |
| THC2407334 | n.s. | n.s. | 4.7 | 3.2 | n.s. |
| THC2407737 | n.s. | n.s. | n.s. | n.s. | 3.6 |
| THC2408033 | n.s. | n.s. | n.s. | 3.4 | n.s. |
| THC2408757 | n.s. | n.s. | n.s. | −3.9 | n.s. |
| THC2408828 | n.s. | n.s. | n.s. | 2.0 | 2.3 |
| THC2409451 | n.s. | n.s. | 1.9 | n.s. | n.s. |
| THC2411515 | n.s. | n.s. | n.s. | n.s. | 1.9 |
| THC2419011 | n.s. | n.s. | 2.3 | n.s. | 1.9 |
| THC2437177 | n.s. | n.s. | 2.5 | 3.3 | 2.2 |
| THC2439773 | n.s. | n.s. | 1.9 | n.s. | n.s. |
| THC2440027 | n.s. | n.s. | n.s. | 2.4 | 3.2 |
| THC2441367 | n.s. | n.s. | 1.8 | n.s. | n.s. |
| THC2448178 | n.s. | n.s. | n.s. | n.s. | 2.7 |
| THC2449905 | n.s. | n.s. | 2.1 | n.s. | n.s. |
| THC2453866 | n.s. | n.s. | 2.1 | n.s. | n.s. |
| THC2455353 | n.s. | n.s. | n.s. | 2.2 | n.s. |
| THEM4 | 1.9 | n.s. | n.s. | n.s. | n.s. |
| THOC4 | 2.7 | n.s. | n.s. | n.s. | n.s. |
| THOC6 | n.s. | −2.6 | n.s. | n.s. | n.s. |
| THOP1 | 2.0 | 1.8 | n.s. | n.s. | n.s. |
| THRAP2 | n.s. | 2.2 | n.s. | n.s. | n.s. |
| THRAP3 | 2.0 | n.s. | n.s. | n.s. | n.s. |
| THRB | −13.3 | −4.7 | n.s. | n.s. | n.s. |
| THSD1 | 2.1 | n.s. | n.s. | n.s. | n.s. |
| THSD4 | −3.2 | n.s. | n.s. | n.s. | n.s. |
| THYN1 | −1.8 | n.s. | n.s. | n.s. | n.s. |
| TIA1 | −2.0 | n.s. | n.s. | n.s. | n.s. |
| TIAM2 | −3.1 | −2.5 | n.s. | n.s. | n.s. |
| TIGD1L | n.s. | 2.0 | n.s. | n.s. | n.s. |
| TIGD3 | n.s. | n.s. | n.s. | n.s. | 2.3 |
| TIMELESS | 2.4 | n.s. | n.s. | n.s. | n.s. |
| TIMM10 | 2.2 | n.s. | n.s. | n.s. | n.s. |
| TIMM13 | 2.0 | n.s. | n.s. | n.s. | n.s. |
| TIMM50 | 2.3 | n.s. | n.s. | n.s. | n.s. |
| TIMM8A | 1.9 | n.s. | n.s. | n.s. | n.s. |
| TIMP3 | 5.1 | 8.4 | n.s. | n.s. | n.s. |
| TIMP4 | −2.6 | n.s. | n.s. | n.s. | n.s. |
| TIPIN | 2.0 | n.s. | n.s. | n.s. | n.s. |
| TJP2 | n.s. | n.s. | −1.9 | n.s. | −2.2 |
| TK2 | n.s. | −3.8 | n.s. | n.s. | n.s. |
| TLCD1 | 1.8 | n.s. | n.s. | n.s. | n.s. |
| TLE3 | 1.9 | 2.1 | n.s. | n.s. | n.s. |
| TLE4 | 2.2 | n.s. | n.s. | n.s. | n.s. |
| TLK1 | n.s. | n.s. | n.s. | n.s. | 1.9 |
| TLN1 | n.s. | 2.0 | n.s. | n.s. | n.s. |
| TLN2 | n.s. | 2.6 | n.s. | n.s. | n.s. |
| TLOC1 | −2.2 | n.s. | n.s. | n.s. | n.s. |
| TM2D1 | −3.6 | −2.4 | n.s. | n.s. | n.s. |
| TM4SF1 | 7.7 | 2.3 | n.s. | n.s. | n.s. |
| TM4SF4 | n.s. | n.s. | n.s. | 1.8 | n.s. |
| TM7SF3 | −2.4 | n.s. | n.s. | n.s. | n.s. |
| TMBIM1 | n.s. | n.s. | −1.9 | n.s. | n.s. |
| TMC8 | n.s. | n.s. | n.s. | n.s. | −3.2 |
| TMCC1 | −2.5 | −1.9 | n.s. | n.s. | n.s. |
| TMCO3 | −1.9 | n.s. | n.s. | n.s. | n.s. |
| TMCO7 | n.s. | 1.8 | n.s. | n.s. | n.s. |
| TMED4 | −1.9 | n.s. | n.s. | n.s. | n.s. |
| TMEM100 | −2.1 | n.s. | n.s. | n.s. | n.s. |
| TMEM103 | 3.1 | 1.9 | n.s. | n.s. | n.s. |
| TMEM106B | −2.3 | n.s. | n.s. | n.s. | n.s. |
| TMEM107 | n.s. | −1.8 | n.s. | n.s. | n.s. |
| TMEM109 | n.s. | n.s. | −2.2 | −1.9 | −2.0 |
| TMEM110 | 2.0 | n.s. | n.s. | n.s. | n.s. |
| TMEM112 | −2.1 | n.s. | n.s. | n.s. | n.s. |
| TMEM117 | 2.2 | 2.6 | n.s. | n.s. | n.s. |
| TMEM119 | −2.1 | n.s. | n.s. | n.s. | n.s. |
| TMEM132D | −2.3 | n.s. | n.s. | n.s. | n.s. |
| TMEM140 | n.s. | −4.4 | n.s. | n.s. | n.s. |
| TMEM150 | n.s. | 2.7 | n.s. | n.s. | n.s. |
| TMEM154 | n.s. | n.s. | n.s. | 1.9 | 1.9 |
| TMEM158 | 1.9 | 3.0 | n.s. | n.s. | n.s. |
| TMEM162 | n.s. | n.s. | 4.7 | n.s. | 3.4 |
| TMEM166 | n.s. | 2.1 | n.s. | n.s. | n.s. |
| TMEM168 | −2.0 | n.s. | n.s. | n.s. | n.s. |
| TMEM170 | 2.2 | n.s. | n.s. | n.s. | n.s. |
| TMEM171 | −2.4 | −1.9 | n.s. | n.s. | n.s. |
| TMEM19 | −2.0 | −2.2 | n.s. | n.s. | n.s. |
| TMEM22 | 2.2 | 2.0 | n.s. | n.s. | n.s. |
| TMEM29 | −1.9 | n.s. | n.s. | n.s. | n.s. |
| TMEM30A | −1.8 | n.s. | n.s. | n.s. | n.s. |
| TMEM30B | −2.9 | −1.9 | n.s. | n.s. | n.s. |
| TMEM33 | 1.9 | n.s. | n.s. | n.s. | n.s. |
| TMEM35 | −3.2 | −2.1 | n.s. | n.s. | n.s. |
| TMEM37 | −5.7 | −7.9 | n.s. | n.s. | n.s. |
| TMEM38B | 3.7 | n.s. | n.s. | n.s. | n.s. |
| TMEM39A | −2.1 | n.s. | n.s. | n.s. | n.s. |
| TMEM42 | −2.0 | −1.8 | n.s. | n.s. | n.s. |
| TMEM46 | 5.0 | n.s. | n.s. | n.s. | n.s. |
| TMEM47 | −2.0 | n.s. | n.s. | n.s. | n.s. |
| TMEM48 | 2.4 | n.s. | n.s. | n.s. | n.s. |
| TMEM57 | 1.9 | n.s. | n.s. | n.s. | n.s. |
| TMEM63A | −5.7 | n.s. | n.s. | n.s. | n.s. |
| TMEM67 | −3.1 | n.s. | n.s. | n.s. | n.s. |
| TMEM81 | n.s. | n.s. | n.s. | −1.8 | n.s. |
| TMEPAI | 5.9 | 4.0 | n.s. | n.s. | n.s. |
| TMF1 | −1.9 | −2.1 | n.s. | n.s. | n.s. |
| TMLHE | −3.5 | −2.2 | n.s. | n.s. | n.s. |
| TMPO | 2.2 | n.s. | n.s. | n.s. | n.s. |
| TMTC1 | −2.1 | n.s. | n.s. | n.s. | n.s. |
| TMTC2 | −2.8 | n.s. | n.s. | n.s. | n.s. |
| TMTC3 | −1.9 | n.s. | n.s. | n.s. | n.s. |
| TMTC4 | −2.4 | n.s. | n.s. | n.s. | n.s. |
| TNC | −2.5 | n.s. | n.s. | n.s. | n.s. |
| TncRNA | 3.1 | −2.3 | n.s. | n.s. | n.s. |
| TNFAIP3 | 7.7 | 8.3 | n.s. | n.s. | n.s. |
| TNFAIP6 | 7.8 | n.s. | n.s. | n.s. | n.s. |
| TNFAIP8L1 | n.s. | −3.4 | n.s. | n.s. | n.s. |
| TNFRSF10A | 3.7 | 2.3 | n.s. | n.s. | n.s. |
| TNFRSF10B | 2.1 | n.s. | n.s. | n.s. | n.s. |
| TNFRSF10D | 4.5 | 2.0 | n.s. | n.s. | n.s. |
| TNFRSF11B | −3.2 | n.s. | n.s. | n.s. | n.s. |
| TNFRSF12A | 2.4 | 1.9 | n.s. | n.s. | n.s. |
| TNFRSF19 | −2.7 | −2.1 | n.s. | n.s. | n.s. |
| TNFRSF1B | n.s. | 2.7 | n.s. | n.s. | n.s. |
| TNFRSF21 | 2.4 | n.s. | n.s. | −2.2 | n.s. |
| TNFRSF25 | n.s. | −3.1 | n.s. | n.s. | n.s. |
| TNFRSF6B | 2.0 | n.s. | n.s. | n.s. | n.s. |
| TNFSF12 | −2.4 | n.s. | n.s. | n.s. | n.s. |
| TNFSF4 | −3.2 | n.s. | n.s. | n.s. | n.s. |
| TNFSF9 | 3.6 | n.s. | n.s. | n.s. | n.s. |
| TNIP2 | n.s. | n.s. | −2.1 | n.s. | n.s. |
| TNKS | 2.2 | n.s. | n.s. | n.s. | n.s. |
| TNKS1BP1 | −2.5 | n.s. | n.s. | n.s. | n.s. |
| TNNC1 | 3.2 | 5.7 | n.s. | n.s. | n.s. |
| TNNC2 | −4.4 | −5.9 | n.s. | n.s. | n.s. |
| TNRC15 | −2.0 | n.s. | n.s. | n.s. | n.s. |
| TNRC4 | n.s. | n.s. | n.s. | n.s. | −2.4 |
| TNRC6A | −2.1 | −2.0 | n.s. | n.s. | n.s. |
| TNRC6B | −2.1 | −2.1 | n.s. | n.s. | n.s. |
| TNRC6C | −1.8 | −3.1 | n.s. | n.s. | n.s. |
| TNRC8 | −2.1 | n.s. | n.s. | n.s. | n.s. |
| TNS3 | −1.9 | n.s. | n.s. | n.s. | n.s. |
| TNXB | −9.5 | 9.5 | n.s. | n.s. | n.s. |
| TOB1 | n.s. | −2.5 | n.s. | n.s. | n.s. |
| TOE1 | 2.2 | n.s. | n.s. | n.s. | n.s. |
| TOLLIP | 22.8 | n.s. | n.s. | n.s. | n.s. |
| TOM1 | 2.2 | n.s. | −2.3 | −1.9 | n.s. |
| TOMM34 | 1.9 | n.s. | −1.8 | n.s. | n.s. |
| TOMM40 | 2.4 | n.s. | n.s. | n.s. | n.s. |
| TOP1 | 1.9 | n.s. | n.s. | n.s. | n.s. |
| TOP2A | 3.1 | n.s. | n.s. | n.s. | n.s. |
| TOX | −3.7 | −2.4 | n.s. | n.s. | n.s. |
| TOX2 | n.s. | 2.0 | n.s. | n.s. | n.s. |
| TP53 | −2.1 | n.s. | n.s. | n.s. | n.s. |
| TP53AP1 | −1.8 | n.s. | n.s. | n.s. | n.s. |
| TP53INP1 | n.s. | −2.6 | n.s. | n.s. | n.s. |
| TP53INP2 | n.s. | n.s. | −1.8 | n.s. | n.s. |
| TPARL | n.s. | 2.2 | n.s. | n.s. | n.s. |
| TPCN1 | −2.9 | n.s. | −1.8 | n.s. | n.s. |
| TPCN2 | n.s. | n.s. | −1.8 | n.s. | n.s. |
| TPD52L1 | −1.9 | n.s. | n.s. | n.s. | n.s. |
| TPI1 | 3.1 | n.s. | n.s. | n.s. | n.s. |
| TPM1 | 2.1 | 4.1 | n.s. | n.s. | n.s. |
| TPM2 | 2.5 | n.s. | n.s. | n.s. | n.s. |
| TPM4 | n.s. | 2.3 | n.s. | n.s. | n.s. |
| TPR | −1.8 | −2.5 | n.s. | n.s. | n.s. |
| TPX2 | 3.6 | n.s. | n.s. | n.s. | n.s. |
| TRA16 | 4.4 | 4.0 | n.s. | n.s. | n.s. |
| TRA2A | n.s. | 3.7 | n.s. | n.s. | n.s. |
| TRABD | 2.4 | n.s. | n.s. | n.s. | n.s. |
| TRAF3 | 2.2 | n.s. | n.s. | n.s. | n.s. |
| TRAF3IP1 | −1.9 | −2.8 | n.s. | n.s. | n.s. |
| TRAF3IP2 | −1.8 | n.s. | n.s. | n.s. | n.s. |
| TRAF5 | −1.9 | n.s. | n.s. | n.s. | n.s. |
| TRAFD1 | n.s. | n.s. | −2.0 | n.s. | n.s. |
| TRAIP | 4.2 | n.s. | n.s. | n.s. | n.s. |
| TRAK1 | −4.3 | n.s. | n.s. | n.s. | n.s. |
| TRAM2 | −1.9 | n.s. | n.s. | n.s. | n.s. |
| TRAPPC2 | −2.3 | n.s. | n.s. | n.s. | n.s. |
| TRAPPC6A | −3.0 | n.s. | n.s. | n.s. | n.s. |
| TRERF1 | −3.7 | n.s. | n.s. | n.s. | n.s. |
| TRIB1 | 7.1 | 5.8 | n.s. | n.s. | n.s. |
| TRIB2 | −2.7 | 2.0 | n.s. | −1.8 | n.s. |
| TRIM13 | −2.7 | n.s. | n.s. | n.s. | n.s. |
| TRIM16 | n.s. | −2.7 | n.s. | n.s. | n.s. |
| TRIM2 | −2.4 | n.s. | n.s. | n.s. | n.s. |
| TRIM21 | −1.8 | n.s. | n.s. | n.s. | n.s. |
| TRIM22 | −3.3 | −2.2 | n.s. | n.s. | n.s. |
| TRIM23 | n.s. | n.s. | 1.9 | n.s. | 1.9 |
| TRIM33 | −2.8 | −2.3 | n.s. | n.s. | n.s. |
| TRIM4 | 2.1 | n.s. | n.s. | n.s. | n.s. |
| TRIM44 | −2.1 | n.s. | n.s. | n.s. | n.s. |
| TRIM45 | n.s. | −2.2 | n.s. | n.s. | n.s. |
| TRIM5 | −2.3 | n.s. | n.s. | n.s. | n.s. |
| TRIM56 | −2.2 | −1.9 | n.s. | n.s. | n.s. |
| TRIM59 | n.s. | −1.9 | n.s. | 2.4 | n.s. |
| TRIM69 | n.s. | −2.4 | n.s. | n.s. | n.s. |
| TRIM73 | n.s. | 2.3 | n.s. | n.s. | n.s. |
| TRIO | n.s. | 3.9 | n.s. | n.s. | n.s. |
| TRIOBP | −1.9 | n.s. | n.s. | n.s. | n.s. |
| TRIP11 | −1.9 | n.s. | n.s. | n.s. | n.s. |
| TRIP12 | −2.4 | n.s. | n.s. | n.s. | n.s. |
| TRIP13 | 3.9 | n.s. | n.s. | n.s. | n.s. |
| TRMT1 | 2.1 | 2.3 | n.s. | n.s. | n.s. |
| TRMT6 | 2.5 | n.s. | n.s. | n.s. | n.s. |
| TRO | −1.9 | n.s. | n.s. | n.s. | n.s. |
| TROAP | 3.0 | −2.0 | n.s. | n.s. | n.s. |
| TROVE2 | −2.6 | −2.1 | n.s. | n.s. | n.s. |
| TRPC1 | −1.9 | n.s. | n.s. | n.s. | n.s. |
| TRPM7 | 9.8 | n.s. | n.s. | n.s. | n.s. |
| TRPS1 | −1.9 | −2.7 | n.s. | n.s. | n.s. |
| TRPV2 | n.s. | 2.9 | n.s. | n.s. | n.s. |
| TRY6 | 3.8 | 1.9 | n.s. | n.s. | n.s. |
| TSC22D3 | 2.2 | n.s. | n.s. | n.s. | n.s. |
| TSC22D4 | −3.6 | n.s. | n.s. | n.s. | n.s. |
| TSEN54 | 1.8 | n.s. | n.s. | n.s. | n.s. |
| TSGA10 | n.s. | n.s. | 3.9 | n.s. | n.s. |
| TSHZ1 | n.s. | −2.9 | n.s. | n.s. | n.s. |
| TSHZ2 | −3.4 | −1.8 | n.s. | n.s. | n.s. |
| TSHZ3 | n.s. | 3.3 | n.s. | n.s. | n.s. |
| TSPAN13 | 5.0 | n.s. | n.s. | n.s. | n.s. |
| TSPAN14 | −1.9 | n.s. | n.s. | n.s. | n.s. |
| TSPAN18 | −3.1 | n.s. | n.s. | −1.9 | n.s. |
| TSPAN2 | 3.7 | 2.8 | n.s. | n.s. | n.s. |
| TSPAN9 | −2.2 | n.s. | n.s. | n.s. | n.s. |
| TSR1 | 2.5 | 2.6 | n.s. | n.s. | n.s. |
| TSR2 | n.s. | n.s. | −2.4 | n.s. | n.s. |
| TSTA3 | 2.0 | n.s. | n.s. | n.s. | n.s. |
| TTBK2 | −1.8 | −2.8 | n.s. | n.s. | n.s. |
| TTC12 | −5.3 | n.s. | n.s. | n.s. | n.s. |
| TTC28 | −3.1 | n.s. | n.s. | n.s. | n.s. |
| TTC3 | −2.1 | n.s. | n.s. | n.s. | n.s. |
| TTC30B | −2.1 | −1.9 | n.s. | n.s. | n.s. |
| TTC32 | n.s. | −2.4 | n.s. | n.s. | n.s. |
| TTK | 2.9 | n.s. | n.s. | n.s. | n.s. |
| TTL | n.s. | 1.9 | n.s. | n.s. | n.s. |
| TTLL12 | 2.0 | 2.0 | n.s. | n.s. | n.s. |
| TTLL3 | −2.6 | n.s. | n.s. | n.s. | n.s. |
| TTRAP | 1.9 | n.s. | n.s. | n.s. | n.s. |
| TTTY14 | n.s. | n.s. | −2.8 | n.s. | −3.1 |
| TUB | −1.9 | n.s. | −2.6 | −3.0 | n.s. |
| TUBA4A | 2.7 | n.s. | n.s. | n.s. | n.s. |
| TUBB2A | 2.5 | n.s. | n.s. | n.s. | n.s. |
| TUBB3 | 2.3 | n.s. | n.s. | n.s. | n.s. |
| TUBB4 | n.s. | n.s. | n.s. | n.s. | 3.9 |
| TUBG1 | 2.4 | n.s. | n.s. | n.s. | n.s. |
| TUG1 | −1.9 | n.s. | n.s. | n.s. | n.s. |
| TUSC3 | −1.8 | n.s. | n.s. | n.s. | n.s. |
| TWIST2 | −2.2 | −2.0 | n.s. | n.s. | n.s. |
| TXLNA | −1.9 | −2.2 | n.s. | n.s. | n.s. |
| TXN2 | n.s. | −2.0 | n.s. | n.s. | n.s. |
| TXNDC4 | n.s. | n.s. | n.s. | 2.0 | 2.7 |
| TXNIP | −2.0 | n.s. | n.s. | n.s. | n.s. |
| TXNL4B | 2.6 | n.s. | n.s. | n.s. | n.s. |
| TXNRD1 | 2.6 | n.s. | n.s. | −1.8 | n.s. |
| TYRO3 | n.s. | 1.9 | n.s. | n.s. | n.s. |
| U87972 | n.s. | n.s. | −2.1 | n.s. | n.s. |
| UACA | −2.2 | n.s. | n.s. | n.s. | n.s. |
| UAP1 | n.s. | 1.9 | n.s. | n.s. | n.s. |
| UAP1L1 | 2.3 | n.s. | n.s. | n.s. | n.s. |
| UBE1L | −2.5 | n.s. | n.s. | n.s. | n.s. |
| UBE1L2 | n.s. | 1.8 | n.s. | n.s. | n.s. |
| UBE2C | 4.7 | n.s. | n.s. | n.s. | n.s. |
| UBE2E1 | n.s. | n.s. | n.s. | n.s. | −2.0 |
| UBE2H | 2.3 | n.s. | n.s. | n.s. | n.s. |
| UBE2M | 1.8 | n.s. | n.s. | n.s. | n.s. |
| UBE2NL | n.s. | 1.9 | n.s. | n.s. | n.s. |
| UBE2S | 2.9 | n.s. | n.s. | n.s. | n.s. |
| UBE2T | 7.2 | n.s. | n.s. | n.s. | n.s. |
| UBE2V2 | −2.2 | −2.0 | n.s. | n.s. | n.s. |
| UBE4B | −1.8 | n.s. | n.s. | n.s. | n.s. |
| UBIAD1 | 2.3 | n.s. | n.s. | n.s. | n.s. |
| UBL3 | −2.8 | n.s. | n.s. | n.s. | n.s. |
| UBN1 | n.s. | −1.9 | n.s. | n.s. | n.s. |
| UBQLN1 | 2.0 | n.s. | n.s. | n.s. | n.s. |
| UBQLN4 | 2.0 | n.s. | n.s. | n.s. | n.s. |
| UBQLNL | n.s. | n.s. | 2.6 | n.s. | n.s. |
| UBR2 | −2.6 | n.s. | n.s. | n.s. | n.s. |
| UBR4 | 1.9 | n.s. | n.s. | n.s. | n.s. |
| UBXD1 | −1.8 | −2.4 | n.s. | n.s. | n.s. |
| UBXD5 | n.s. | −3.3 | n.s. | n.s. | n.s. |
| UBXD7 | n.s. | −1.9 | n.s. | n.s. | n.s. |
| UCHL5 | 1.9 | n.s. | n.s. | n.s. | n.s. |
| UCK2 | 3.4 | 2.3 | n.s. | n.s. | n.s. |
| UEV3 | −3.5 | −2.0 | n.s. | n.s. | n.s. |
| UGCG | −2.3 | n.s. | n.s. | n.s. | n.s. |
| UHRF1 | 2.2 | 2.6 | n.s. | n.s. | n.s. |
| ULBP2 | 2.8 | n.s. | n.s. | n.s. | n.s. |
| ULK2 | −1.9 | n.s. | n.s. | n.s. | n.s. |
| UNC119 | n.s. | −2.8 | n.s. | n.s. | n.s. |
| UNC84A | −2.1 | n.s. | n.s. | n.s. | n.s. |
| UNC84B | n.s. | n.s. | −2.0 | n.s. | n.s. |
| UNG | n.s. | 1.9 | n.s. | n.s. | n.s. |
| UNKL | n.s. | −1.9 | n.s. | n.s. | n.s. |
| unknown | n.s. | −2.4 | n.s. | n.s. | n.s. |
| UNQ338 | n.s. | −2.0 | n.s. | n.s. | n.s. |
| UPP1 | 3.1 | 3.3 | n.s. | n.s. | n.s. |
| URLC9 | 3.5 | n.s. | n.s. | n.s. | n.s. |
| USF2 | n.s. | n.s. | n.s. | 1.9 | n.s. |
| USP10 | n.s. | 1.8 | n.s. | n.s. | n.s. |
| USP21 | −2.4 | −2.2 | n.s. | n.s. | n.s. |
| USP25 | −2.1 | n.s. | n.s. | n.s. | n.s. |
| USP3 | −2.6 | n.s. | n.s. | n.s. | n.s. |
| USP30 | −1.8 | n.s. | n.s. | n.s. | n.s. |
| USP32 | −2.0 | n.s. | n.s. | n.s. | n.s. |
| USP34 | −2.3 | n.s. | 1.8 | n.s. | n.s. |
| USP36 | 2.0 | n.s. | n.s. | n.s. | n.s. |
| USP45 | n.s. | −2.0 | n.s. | n.s. | n.s. |
| USP47 | −1.9 | n.s. | n.s. | n.s. | n.s. |
| USP52 | −1.8 | n.s. | n.s. | n.s. | n.s. |
| USP53 | −2.4 | −2.3 | n.s. | n.s. | n.s. |
| USP54 | −1.9 | n.s. | n.s. | n.s. | n.s. |
| USP6NL | n.s. | n.s. | 1.9 | 1.8 | n.s. |
| USP7 | n.s. | 1.9 | n.s. | n.s. | n.s. |
| USP9X | n.s. | −2.9 | n.s. | n.s. | n.s. |
| UST | 3.4 | n.s. | n.s. | n.s. | n.s. |
| UTP15 | n.s. | 1.8 | n.s. | n.s. | n.s. |
| UTS2D | n.s. | n.s. | 2.1 | n.s. | 1.8 |
| VAC14 | n.s. | −2.2 | n.s. | n.s. | n.s. |
| VAMP4 | −2.1 | n.s. | n.s. | n.s. | n.s. |
| VAPA | n.s. | −1.9 | n.s. | n.s. | n.s. |
| VAPB | n.s. | 2.1 | n.s. | n.s. | n.s. |
| VARS | 1.8 | 2.1 | n.s. | n.s. | n.s. |
| VASH1 | −2.5 | n.s. | n.s. | n.s. | n.s. |
| VASP | n.s. | 2.2 | n.s. | n.s. | n.s. |
| VCAN | −1.9 | 2.0 | n.s. | n.s. | n.s. |
| VCP | n.s. | n.s. | −2.2 | −1.8 | −1.9 |
| VCPIP1 | n.s. | −2.4 | n.s. | n.s. | n.s. |
| VDP | −2.2 | −1.8 | n.s. | n.s. | n.s. |
| VDR | −2.2 | n.s. | n.s. | n.s. | n.s. |
| VEGFA | 1.9 | n.s. | n.s. | n.s. | n.s. |
| VEGFC | n.s. | 2.3 | n.s. | n.s. | n.s. |
| VEPH1 | 2.5 | n.s. | n.s. | n.s. | n.s. |
| VGLL3 | −4.4 | −1.8 | n.s. | n.s. | n.s. |
| VIL2 | 2.8 | 2.2 | n.s. | n.s. | n.s. |
| VIM | −2.1 | n.s. | n.s. | n.s. | n.s. |
| VISA | −2.9 | −2.3 | n.s. | n.s. | n.s. |
| VIT | −2.3 | n.s. | n.s. | n.s. | n.s. |
| VMD2 | −2.7 | n.s. | n.s. | n.s. | n.s. |
| VPRBP | n.s. | n.s. | −2.3 | n.s. | n.s. |
| VPS13A | −2.0 | n.s. | n.s. | n.s. | n.s. |
| VPS13B | −2.8 | n.s. | n.s. | n.s. | n.s. |
| VPS13C | −3.1 | n.s. | n.s. | n.s. | n.s. |
| VPS13D | −2.7 | n.s. | n.s. | n.s. | n.s. |
| VPS24 | −1.9 | n.s. | n.s. | n.s. | n.s. |
| VPS36 | −2.1 | n.s. | n.s. | n.s. | n.s. |
| VPS41 | −2.2 | n.s. | n.s. | n.s. | n.s. |
| VPS4A | −2.1 | −2.2 | n.s. | n.s. | n.s. |
| VPS53 | n.s. | −1.9 | n.s. | n.s. | n.s. |
| VRK1 | 3.3 | n.s. | n.s. | n.s. | n.s. |
| VSIG8 | −2.1 | n.s. | n.s. | n.s. | n.s. |
| VTI1A | 2.1 | n.s. | n.s. | n.s. | n.s. |
| W05707 | n.s. | n.s. | n.s. | n.s. | 1.9 |
| WAPAL | 1.9 | n.s. | n.s. | n.s. | n.s. |
| WASF2 | n.s. | −1.8 | n.s. | n.s. | n.s. |
| WBP1 | −2.0 | n.s. | n.s. | n.s. | n.s. |
| WDFY2 | n.s. | 3.9 | n.s. | n.s. | n.s. |
| WDFY3 | −2.0 | n.s. | n.s. | n.s. | n.s. |
| WDHD1 | 2.7 | 1.8 | n.s. | n.s. | n.s. |
| WDR13 | −1.9 | n.s. | n.s. | n.s. | n.s. |
| WDR19 | −2.3 | n.s. | n.s. | n.s. | n.s. |
| WDR31 | n.s. | n.s. | −2.0 | n.s. | −2.2 |
| WDR32 | −2.2 | −2.0 | n.s. | n.s. | n.s. |
| WDR4 | 2.1 | 2.3 | n.s. | n.s. | n.s. |
| WDR45 | 2.0 | 1.8 | n.s. | n.s. | n.s. |
| WDR5 | 2.9 | n.s. | n.s. | n.s. | n.s. |
| WDR51A | 3.8 | n.s. | n.s. | n.s. | n.s. |
| WDR60 | −2.8 | −2.1 | n.s. | n.s. | n.s. |
| WDR68 | 3.4 | n.s. | n.s. | n.s. | n.s. |
| WDR76 | n.s. | 3.1 | n.s. | n.s. | n.s. |
| WDR77 | 2.0 | n.s. | n.s. | n.s. | n.s. |
| WDR79 | 7.5 | n.s. | n.s. | n.s. | n.s. |
| WDR82 | n.s. | n.s. | −1.9 | n.s. | n.s. |
| WFDC1 | 3.6 | 2.3 | n.s. | n.s. | n.s. |
| WFDC3 | 2.6 | n.s. | n.s. | n.s. | n.s. |
| WFS1 | n.s. | 1.8 | n.s. | n.s. | n.s. |
| WHSC1L1 | −2.5 | −2.0 | n.s. | n.s. | n.s. |
| WIPF1 | −2.0 | n.s. | n.s. | n.s. | n.s. |
| WIPF2 | −2.1 | −2.0 | n.s. | n.s. | n.s. |
| WISP1 | −2.3 | n.s. | n.s. | n.s. | n.s. |
| WISP2 | −2.1 | n.s. | n.s. | n.s. | n.s. |
| WNK1 | n.s. | n.s. | n.s. | 4.4 | n.s. |
| WNK4 | n.s. | 3.0 | n.s. | n.s. | n.s. |
| WNT10A | n.s. | n.s. | n.s. | −1.9 | −1.8 |
| WNT11 | −7.5 | −7.7 | n.s. | n.s. | n.s. |
| WNT16 | n.s. | n.s. | n.s. | 5.0 | 2.4 |
| WNT2 | −2.3 | n.s. | n.s. | n.s. | n.s. |
| WNT5A | −2.8 | n.s. | n.s. | n.s. | n.s. |
| WNT5B | 2.1 | 2.4 | n.s. | n.s. | n.s. |
| WRNIP1 | n.s. | n.s. | n.s. | n.s. | 3.1 |
| WSB1 | −3.5 | n.s. | n.s. | n.s. | n.s. |
| WSB2 | 2.3 | n.s. | n.s. | n.s. | n.s. |
| WTAP | n.s. | 2.2 | n.s. | n.s. | n.s. |
| WWC1 | n.s. | 2.9 | n.s. | n.s. | n.s. |
| WWC2 | −1.8 | n.s. | 3.6 | n.s. | n.s. |
| WWOX | −4.0 | n.s. | n.s. | n.s. | n.s. |
| WWTR1 | 2.8 | −1.8 | n.s. | n.s. | n.s. |
| XAF1 | −2.1 | n.s. | n.s. | n.s. | n.s. |
| XDH | n.s. | 2.4 | n.s. | n.s. | n.s. |
| XG | −2.7 | n.s. | n.s. | n.s. | n.s. |
| XIST | −2.0 | n.s. | n.s. | n.s. | n.s. |
| XPA | −2.0 | n.s. | n.s. | n.s. | n.s. |
| XPNPEP3 | −1.9 | −2.7 | n.s. | n.s. | n.s. |
| XPO1 | −2.6 | −2.3 | n.s. | n.s. | n.s. |
| XPO5 | n.s. | 3.0 | n.s. | n.s. | n.s. |
| XPOT | −1.9 | n.s. | n.s. | n.s. | n.s. |
| XRCC4 | n.s. | −1.9 | n.s. | n.s. | n.s. |
| XRCC6BP1 | n.s. | −2.8 | n.s. | n.s. | n.s. |
| XRN1 | −2.2 | n.s. | n.s. | n.s. | n.s. |
| XRRA1 | n.s. | −2.9 | n.s. | n.s. | n.s. |
| XYLT1 | 2.0 | 3.1 | n.s. | n.s. | n.s. |
| YAP1 | 2.1 | −2.1 | n.s. | n.s. | n.s. |
| YIPF6 | −1.9 | n.s. | n.s. | n.s. | n.s. |
| YKT6 | 3.5 | n.s. | −2.0 | n.s. | n.s. |
| YOD1 | 2.1 | n.s. | n.s. | n.s. | n.s. |
| YPEL1 | n.s. | −3.3 | n.s. | n.s. | n.s. |
| YPEL2 | n.s. | −2.8 | n.s. | n.s. | n.s. |
| YPEL3 | n.s. | −2.3 | n.s. | n.s. | n.s. |
| YPEL4 | −3.3 | −3.8 | n.s. | n.s. | n.s. |
| YRDC | 4.3 | 2.1 | n.s. | n.s. | n.s. |
| YTHDC2 | −1.9 | n.s. | n.s. | n.s. | n.s. |
| YTHDF3 | 1.9 | n.s. | n.s. | n.s. | n.s. |
| Z28739 | n.s. | n.s. | n.s. | 1.9 | n.s. |
| ZAK | 1.8 | −1.9 | n.s. | n.s. | n.s. |
| ZBED1 | n.s. | n.s. | n.s. | −2.3 | −2.4 |
| ZBED3 | −2.2 | −1.9 | n.s. | n.s. | n.s. |
| ZBED5 | −1.9 | n.s. | n.s. | n.s. | n.s. |
| ZBTB10 | −1.9 | n.s. | n.s. | n.s. | n.s. |
| ZBTB20 | −1.9 | n.s. | n.s. | n.s. | n.s. |
| ZBTB24 | 2.6 | n.s. | n.s. | n.s. | n.s. |
| ZBTB26 | −2.2 | −2.0 | n.s. | n.s. | n.s. |
| ZBTB3 | n.s. | −2.0 | n.s. | n.s. | n.s. |
| ZBTB34 | n.s. | −1.8 | n.s. | n.s. | n.s. |
| ZBTB41 | n.s. | −2.1 | n.s. | n.s. | n.s. |
| ZBTB43 | 3.7 | n.s. | n.s. | n.s. | n.s. |
| ZBTB44 | −2.3 | −2.0 | n.s. | n.s. | n.s. |
| ZC3H13 | −1.9 | n.s. | n.s. | −1.9 | n.s. |
| ZC3H6 | −5.7 | n.s. | n.s. | n.s. | n.s. |
| ZC3HAV1L | −2.9 | n.s. | n.s. | n.s. | n.s. |
| ZCCHC10 | 2.3 | n.s. | n.s. | n.s. | n.s. |
| ZCCHC11 | −2.1 | n.s. | n.s. | n.s. | n.s. |
| ZCCHC3 | n.s. | n.s. | −1.9 | n.s. | n.s. |
| ZDBF2 | n.s. | n.s. | n.s. | 1.9 | n.s. |
| ZDHHC14 | −3.1 | n.s. | n.s. | n.s. | n.s. |
| ZDHHC17 | n.s. | n.s. | n.s. | −1.9 | n.s. |
| ZDHHC2 | −2.5 | n.s. | n.s. | n.s. | n.s. |
| ZDHHC21 | −2.5 | −2.2 | n.s. | n.s. | n.s. |
| ZDHHC22 | n.s. | n.s. | n.s. | −2.8 | −2.5 |
| ZDHHC3 | −1.9 | n.s. | n.s. | n.s. | n.s. |
| ZDHHC5 | n.s. | 2.2 | n.s. | n.s. | n.s. |
| ZEB1 | 8.7 | 6.0 | n.s. | n.s. | n.s. |
| ZEB2 | −1.9 | n.s. | n.s. | n.s. | n.s. |
| ZFAND2A | 2.4 | n.s. | n.s. | n.s. | n.s. |
| ZFAND2B | 1.9 | n.s. | n.s. | n.s. | n.s. |
| ZFAND5 | 2.2 | n.s. | n.s. | n.s. | n.s. |
| ZFAND6 | n.s. | 2.7 | n.s. | n.s. | n.s. |
| ZFHX3 | −2.0 | n.s. | n.s. | n.s. | n.s. |
| ZFHX4 | −2.1 | −2.1 | n.s. | n.s. | n.s. |
| ZFP1 | −2.1 | n.s. | n.s. | n.s. | n.s. |
| ZFP106 | −2.1 | −1.9 | n.s. | n.s. | n.s. |
| ZFP3 | −3.4 | n.s. | n.s. | n.s. | n.s. |
| ZFP36L2 | −3.1 | −2.1 | n.s. | n.s. | n.s. |
| ZFP90 | −2.0 | n.s. | n.s. | n.s. | n.s. |
| ZFPL1 | n.s. | −2.0 | n.s. | n.s. | n.s. |
| ZFPM2 | n.s. | n.s. | 2.0 | n.s. | n.s. |
| ZFX | n.s. | −1.9 | n.s. | n.s. | n.s. |
| ZFY | 2.1 | n.s. | n.s. | n.s. | n.s. |
| ZFYVE16 | −5.5 | n.s. | n.s. | n.s. | n.s. |
| ZGPAT | 1.9 | n.s. | n.s. | n.s. | n.s. |
| ZHX1 | −1.8 | n.s. | n.s. | n.s. | n.s. |
| ZHX2 | −2.7 | n.s. | n.s. | n.s. | n.s. |
| ZHX3 | −3.0 | n.s. | n.s. | n.s. | n.s. |
| ZIK1 | 2.3 | n.s. | n.s. | n.s. | n.s. |
| ZKSCAN1 | −2.0 | −1.8 | n.s. | n.s. | n.s. |
| ZMAT3 | −2.1 | −1.8 | n.s. | n.s. | n.s. |
| ZMIZ1 | n.s. | 3.7 | n.s. | 2.1 | n.s. |
| ZMYM2 | −2.6 | n.s. | n.s. | n.s. | n.s. |
| ZMYM4 | −2.0 | n.s. | n.s. | n.s. | n.s. |
| ZMYM5 | n.s. | −2.0 | n.s. | n.s. | n.s. |
| ZMYND10 | −2.1 | n.s. | n.s. | n.s. | n.s. |
| ZMYND11 | −2.1 | n.s. | n.s. | n.s. | n.s. |
| ZNF101 | 2.0 | n.s. | n.s. | n.s. | n.s. |
| ZNF107 | n.s. | −2.4 | n.s. | n.s. | n.s. |
| ZNF117 | −2.1 | n.s. | n.s. | n.s. | n.s. |
| ZNF12 | n.s. | −2.3 | n.s. | n.s. | n.s. |
| ZNF131 | n.s. | 2.2 | n.s. | n.s. | n.s. |
| ZNF14 | n.s. | −3.0 | n.s. | n.s. | n.s. |
| ZNF148 | −1.9 | −1.9 | n.s. | n.s. | n.s. |
| ZNF160 | −2.1 | n.s. | n.s. | n.s. | n.s. |
| ZNF165 | n.s. | −3.7 | n.s. | n.s. | n.s. |
| ZNF167 | −2.0 | n.s. | n.s. | n.s. | n.s. |
| ZNF174 | n.s. | −1.9 | n.s. | n.s. | n.s. |
| ZNF182 | n.s. | −1.8 | n.s. | n.s. | n.s. |
| ZNF189 | −2.2 | −2.2 | n.s. | n.s. | n.s. |
| ZNF19 | n.s. | −2.1 | n.s. | n.s. | n.s. |
| ZNF192 | n.s. | −1.8 | n.s. | n.s. | n.s. |
| ZNF20 | n.s. | −2.3 | n.s. | n.s. | n.s. |
| ZNF207 | −3.3 | −4.7 | n.s. | n.s. | n.s. |
| ZNF217 | −2.6 | −1.8 | n.s. | n.s. | n.s. |
| ZNF223 | n.s. | −2.4 | n.s. | n.s. | n.s. |
| ZNF224 | n.s. | n.s. | 7.6 | 12.2 | 6.2 |
| ZNF225 | −2.6 | −1.8 | n.s. | n.s. | n.s. |
| ZNF226 | −2.1 | −1.8 | n.s. | n.s. | n.s. |
| ZNF228 | −1.9 | n.s. | n.s. | n.s. | n.s. |
| ZNF230 | n.s. | −2.6 | n.s. | n.s. | n.s. |
| ZNF232 | 2.1 | n.s. | n.s. | n.s. | n.s. |
| ZNF236 | n.s. | n.s. | n.s. | 1.9 | n.s. |
| ZNF24 | 2.2 | n.s. | n.s. | n.s. | n.s. |
| ZNF248 | −2.3 | n.s. | n.s. | n.s. | n.s. |
| ZNF252 | −2.4 | n.s. | n.s. | n.s. | n.s. |
| ZNF253 | n.s. | −2.0 | n.s. | n.s. | n.s. |
| ZNF259 | 1.9 | n.s. | n.s. | n.s. | n.s. |
| ZNF264 | n.s. | −2.5 | n.s. | n.s. | n.s. |
| ZNF267 | n.s. | −2.3 | n.s. | n.s. | n.s. |
| ZNF273 | n.s. | −2.1 | n.s. | n.s. | n.s. |
| ZNF277 | 3.7 | −1.8 | n.s. | n.s. | n.s. |
| ZNF280D | −2.5 | n.s. | n.s. | n.s. | n.s. |
| ZNF282 | n.s. | n.s. | −1.8 | n.s. | n.s. |
| ZNF286A | n.s. | −2.4 | n.s. | n.s. | n.s. |
| ZNF289 | n.s. | n.s. | −2.5 | n.s. | −1.8 |
| ZNF292 | −2.3 | −3.0 | n.s. | n.s. | n.s. |
| ZNF294 | n.s. | 1.9 | n.s. | n.s. | n.s. |
| ZNF297B | 3.4 | n.s. | n.s. | n.s. | n.s. |
| ZNF302 | −1.9 | n.s. | n.s. | n.s. | n.s. |
| ZNF313 | 2.0 | n.s. | n.s. | n.s. | n.s. |
| ZNF317 | 2.4 | n.s. | n.s. | n.s. | n.s. |
| ZNF323 | n.s. | −3.4 | n.s. | n.s. | n.s. |
| ZNF326 | n.s. | −2.4 | n.s. | n.s. | n.s. |
| ZNF331 | n.s. | −2.1 | n.s. | n.s. | n.s. |
| ZNF333 | −2.1 | −2.4 | n.s. | n.s. | n.s. |
| ZNF334 | −3.9 | n.s. | n.s. | n.s. | n.s. |
| ZNF33A | −2.3 | −2.2 | n.s. | n.s. | n.s. |
| ZNF345 | −2.8 | n.s. | n.s. | n.s. | n.s. |
| ZNF347 | n.s. | n.s. | n.s. | 2.1 | 2.1 |
| ZNF350 | n.s. | −1.8 | n.s. | n.s. | n.s. |
| ZNF354C | n.s. | −2.0 | n.s. | n.s. | n.s. |
| ZNF367 | 2.0 | 2.0 | n.s. | n.s. | n.s. |
| ZNF37B | −2.2 | n.s. | n.s. | n.s. | n.s. |
| ZNF395 | −3.0 | −2.3 | n.s. | n.s. | n.s. |
| ZNF397 | −2.1 | −3.8 | n.s. | n.s. | n.s. |
| ZNF404 | −2.1 | −1.8 | n.s. | n.s. | n.s. |
| ZNF408 | 2.1 | n.s. | n.s. | n.s. | n.s. |
| ZNF415 | n.s. | −1.9 | n.s. | n.s. | n.s. |
| ZNF420 | n.s. | −2.3 | n.s. | n.s. | n.s. |
| ZNF423 | −2.1 | n.s. | n.s. | n.s. | n.s. |
| ZNF43 | n.s. | −1.8 | n.s. | n.s. | n.s. |
| ZNF430 | n.s. | −2.7 | n.s. | n.s. | n.s. |
| ZNF432 | n.s. | −1.9 | n.s. | n.s. | n.s. |
| ZNF438 | −2.3 | n.s. | n.s. | n.s. | n.s. |
| ZNF439 | n.s. | −3.3 | n.s. | n.s. | n.s. |
| ZNF440 | n.s. | 2.3 | n.s. | n.s. | n.s. |
| ZNF441 | n.s. | −2.2 | n.s. | n.s. | n.s. |
| ZNF446 | −2.2 | −3.1 | n.s. | n.s. | n.s. |
| ZNF449 | −2.0 | n.s. | n.s. | n.s. | n.s. |
| ZNF462 | −2.7 | n.s. | n.s. | n.s. | n.s. |
| ZNF468 | 1.9 | n.s. | n.s. | n.s. | n.s. |
| ZNF473 | 2.5 | n.s. | n.s. | n.s. | n.s. |
| ZNF512 | −2.7 | n.s. | n.s. | n.s. | n.s. |
| ZNF512B | −2.0 | n.s. | n.s. | n.s. | n.s. |
| ZNF516 | n.s. | n.s. | 2.2 | 3.4 | 1.9 |
| ZNF517 | n.s. | n.s. | n.s. | n.s. | −5.8 |
| ZNF521 | −2.3 | −2.4 | n.s. | n.s. | n.s. |
| ZNF529 | n.s. | −1.9 | n.s. | n.s. | n.s. |
| ZNF532 | −1.8- | 2.1 | n.s. | n.s. | n.s. |
| ZNF551 | 2.2 | n.s. | n.s. | n.s. | n.s. |
| ZNF555 | n.s. | −2.2 | n.s. | n.s. | n.s. |
| ZNF557 | n.s. | −2.4 | n.s. | n.s. | n.s. |
| ZNF560 | n.s. | n.s. | n.s. | n.s. | −5.7 |
| ZNF564 | n.s. | −2.0 | n.s. | n.s. | n.s. |
| ZNF567 | n.s. | −2.0 | n.s. | n.s. | n.s. |
| ZNF569 | 2.2 | n.s. | n.s. | n.s. | n.s. |
| ZNF57 | 4.8 | n.s. | n.s. | n.s. | n.s. |
| ZNF573 | −2.6 | n.s. | n.s. | n.s. | n.s. |
| ZNF585A | n.s. | −2.0 | 3.0 | 2.0 | 2.1 |
| ZNF587 | n.s. | n.s. | 3.3 | n.s. | n.s. |
| ZNF597 | n.s. | n.s. | n.s. | n.s. | 3.4 |
| ZNF599 | −2.7 | −1.9 | n.s. | n.s. | n.s. |
| ZNF605 | −2.1 | −1.9 | n.s. | n.s. | n.s. |
| ZNF606 | −2.1 | n.s. | n.s. | n.s. | n.s. |
| ZNF615 | n.s. | −1.9 | n.s. | n.s. | n.s. |
| ZNF618 | −1.9 | −3.0 | n.s. | n.s. | n.s. |
| ZNF621 | n.s. | −2.0 | n.s. | n.s. | n.s. |
| ZNF622 | 2.2 | n.s. | n.s. | n.s. | n.s. |
| ZNF623 | −5.1 | −2.4 | n.s. | n.s. | n.s. |
| ZNF627 | n.s. | −2.0 | n.s. | n.s. | n.s. |
| ZNF630 | −2.5 | n.s. | n.s. | n.s. | n.s. |
| ZNF652 | −2.0 | −2.5 | n.s. | n.s. | n.s. |
| ZNF655 | −1.8 | n.s. | n.s. | n.s. | n.s. |
| ZNF658 | −6.8 | −3.1 | n.s. | n.s. | n.s. |
| ZNF662 | −2.9 | −2.5 | n.s. | n.s. | n.s. |
| ZNF672 | 1.9 | 2.3 | n.s. | n.s. | n.s. |
| ZNF675 | n.s. | −1.9 | n.s. | n.s. | n.s. |
| ZNF677 | n.s. | −2.3 | n.s. | n.s. | n.s. |
| ZNF680 | n.s. | −2.4 | n.s. | n.s. | n.s. |
| ZNF681 | n.s. | n.s. | n.s. | 2.6 | n.s. |
| ZNF684 | n.s. | −2.6 | n.s. | n.s. | n.s. |
| ZNF688 | n.s. | −1.9 | n.s. | n.s. | n.s. |
| ZNF691 | n.s. | −2.0 | n.s. | n.s. | n.s. |
| ZNF697 | 2.5 | n.s. | n.s. | n.s. | n.s. |
| ZNF70 | −1.8 | −2.0 | n.s. | n.s. | n.s. |
| ZNF702 | n.s. | n.s. | n.s. | 1.9 | n.s. |
| ZNF708 | n.s. | −4.4 | n.s. | n.s. | n.s. |
| ZNF709 | −1.9 | −2.8 | n.s. | n.s. | n.s. |
| ZNF710 | −1.9 | −1.8 | n.s. | n.s. | n.s. |
| ZNF713 | −3.5 | n.s. | n.s. | n.s. | n.s. |
| ZNF717 | −1.9 | n.s. | n.s. | n.s. | n.s. |
| ZNF75 | −1.8 | n.s. | n.s. | n.s. | n.s. |
| ZNF768 | n.s. | n.s. | −2.1 | n.s. | n.s. |
| ZNF770 | n.s. | −2.1 | n.s. | n.s. | n.s. |
| ZNF783 | −2.6 | n.s. | n.s. | n.s. | n.s. |
| ZNF785 | −2.0 | n.s. | n.s. | n.s. | n.s. |
| ZNF792 | n.s. | n.s. | n.s. | n.s. | −1.9 |
| ZNF805 | n.s. | −2.2 | n.s. | n.s. | n.s. |
| ZNF81 | n.s. | −2.2 | n.s. | n.s. | n.s. |
| ZNF814 | −2.0 | −2.7 | n.s. | −3.1 | n.s. |
| ZNF818 | −1.9 | −2.2 | n.s. | n.s. | n.s. |
| ZNF84 | −1.9 | n.s. | n.s. | n.s. | n.s. |
| ZNF85 | 2.7 | n.s. | n.s. | n.s. | n.s. |
| ZNF91 | n.s. | −4.0 | n.s. | n.s. | n.s. |
| ZNF92 | n.s. | −1.8 | n.s. | n.s. | n.s. |
| ZNF93 | 5.0 | n.s. | n.s. | n.s. | n.s. |
| ZRF1 | −2.4 | n.s. | n.s. | n.s. | n.s. |
| ZSCAN29 | n.s. | −1.8 | n.s. | n.s. | n.s. |
| ZSWIM6 | n.s. | −1.8 | n.s. | n.s. | n.s. |
| ZWILCH | 2.5 | n.s. | n.s. | n.s. | n.s. |
| ZWINT | 3.3 | n.s. | n.s. | n.s. | n.s. |
| ZXDB | −1.8 | −1.8 | n.s. | n.s. | n.s. |
| ZYG11B | −2.0 | −2.0 | n.s. | n.s. | n.s. |
| ZYX | n.s. | 2.2 | n.s. | n.s. | n.s. |

I claim:

1. A method for modulating the lifespan of a cell, tissue, organ or organism, or of increasing or decreasing cellular respiration and/or capacity and/or biogenesis of mitochondria in a cell, tissue, organ or organism, comprising contacting the cell, tissue, organ or organism with at least one lifespan modulating agent selected from the group consisting of:
idebenone, or an analog or derivative thereof;
a cocoa extract;
a coffee cherry extract;
quinic acid, or an analog or derivative thereof;
ferulic acid, or an analog or derivative thereof;
a proanthocyanidin, anthocyanidin, procyanidin, or cyanidin;
chlorogenic acid, or an analog or derivative thereof;
a tea extract; or
resveratrol or a composition derived from or chemically related to resveratrol.

2. The method of claim 1, wherein modulating comprises modulating the level and/or activity of:
(a) ten or more of the genes listed as part of Array 2;
(b) the genes listed as part of Array 1;
(c) VEGFA, HMOX1, CCL4L1, DDC, NOS2A, SIRT1, TERT, PTGS2, or IFI44;
(d) four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70;
(e) BCL2, SOD1, TP53, and SOD2;
(f) BCL2, SOD1, TP53, SOD2, BCL2L1, TIMM22, TOMM40, IMMP1L, CDKN2A, GADPH, ACTB, HRP1, and HGDC;
(g) PARP1, PARP2, TERT, TEP1, TPS3, JUN, PARP3, PARP4, TERF2, TINF2, and CDKN2A;
(h) PARP1, PARP2, TERT, TEP1, and TP53;
(i) TERF2, POT1, TERT, and TPP1;
(j) PAPR1, PARP2, PARP3, and PARP4;
(k) PARP2, CYP19A1, TEP1, BCL2, HSPA1A, ACE, TP53, and NFKB1;
(l) IGF1, IGF2, PPARG, IL10, APOE, TERT, TNF, HLA-DRA, DDC, CCL4L1, NOS2A, and GH1;
(m) PARP1, IL6, SIRTT1, KRAS, and HSPA1L;
(n) IGF1, IL6, PPARG, IL10, TERT, TNF, TEP1, HSPA1A, SIRT1, TP53, GH1, NOS2A, and PPC;
(o) another list of genes described herein; or
(p) a combination of two or more of (a) through (o).

3. The method of claim 1, wherein modulating the lifespan comprises modulating the activity or level of at least one of the telomere length maintenance genes or modulating the activity or level of telomerase.

4. A method for modulating response or resistance to stress of a cell, tissue, organ or organism, comprising modulating the level and/or activity of at least one gene selected from the group consisting of those listed in Data Table 7 and those listed as part of Array 2.

5. The method of claim 4, wherein modulating comprises modulating the level and/or activity of:
(a) ten or more of the genes listed as part of Array 2;
(b) the genes listed as part of Array 1;
(c) VEGFA, HMOX1, CCL4L1, DDC, NOS2A, SIRT1, TERT, PTGS2, or IFI44;
(d) four or more of TERT, TERC, NRF2, POT1, TRF1, TRF2, TIN2, TPP1, RAPT, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70;
(e) BCL2, SOD1, TP53, and SOD2;
(f) BCL2, SOD1, TP53, SOD2, BCL2L1, TIMM22, TOMM40, IMMP1L, CDKN2A, GADPH, ACTB, HRP1, and HGDC;
(g) PARP1, PARP2, TERT, TEP1, TPS3, JUN, PARP3, PARP4, TERF2, TINF2, and CDKN2A;
(h) PARP1, PARP2, TERT, TEP1, and TP53;
(i) TERF2, POT1, TERT, and TPP1;
(j) PAPR1, PARP2, PARP3, and PARP4;
(k) PARP2, CYP19A1, TEP1, BCL2, HSPA1A, ACE, TP53, and NFKB1;
(l) IGF1, IGF2, PPARG, IL10, APOE, TERT, TNF, HLA-DRA, DDC, CCL4L1, NOS2A, and GH1;
(m) PARP1, IL6, SIRTT1, KRAS, and HSPA1L;
(n) IGF1, IL6, PPARG, IL10, TERT, TNF, TEP1, HSPA1A, SIRT1, TP53, GH1, NOS2A, and PPC;
(o) another list of genes described herein; or
(p) a combination of two or more of (a) through (o).

6. The method of claim 3, wherein modulating comprises increasing the level of activity of the at least one listed gene.

7. The method of claim 3, wherein modulating comprises decreasing the level of activity of the at least one listed gene.

8. The method of claim 1, wherein the method comprises increasing the lifespan of a cell through modulating biogenesis of, or respiratory efficiency of mitochondria, lengthening telomeres, and/or modulating at least one gene affecting the same.

9. The method of claim 1, comprising increasing or decreasing proliferation or biogenesis of mitochondria through modulation of at least one of PGC 1α, SIRT1, SIRT3, SIRT4, SIRT5, NRF1 and/or Tfam.

10. The method of claim 1, further comprising inducing mitochondrial regeneration, or new mitochondrial biosynthesis in at least one cell.

11. A method for modulating, preventing, delaying, or reversing acute cell death or apoptosis, or prolonging the survival of a cell, tissue, organ or organism comprising modulating the level and/or activity of at least one gene selected from the group consisting of those listed in Data Table 7 and those listed as part of Array 2.

12. The method of claim 11, wherein modulating acute cell death or apoptosis comprises increasing or upregulating acute cell death or apoptosis.

13. A collection of lifespan-influencing nucleic acid molecules, which collection comprises a plurality of nucleic acid molecules selected from those listed in Data Table 7 or Array 2, or fragments of those listed in Data Table 7 or Array 2.

14. The microarray collection of claim 13, which comprises nucleic acid molecules having at least the sequence as set for in:
(a) the genes listed as part of Array 1;
(b) the genes listed as part of Array 2;
(c) VEGFA, HMOX1, CCL4L1, DDC, NOS2A, SIRT1, TERT, PTGS2, or IFI44;
(d) four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70;
(e) BCL2, SOD1, TP53, and SOD2;
(f) BCL2, SOD1, TP53, SOD2, BCL2L1, TIMM22, TOMM40, IMMP1L, CDKN2A, GADPH, ACTB, HRP1, and HGDC;
(g) PARP1, PARP2, TERT, TEP1, TPS3, JUN, PARP3, PARP4, TERF2, TINF2, and CDKN2A;
(h) PARP1, PARP2, TERT, TEP1, and TP53;
(i) TERF2, POT1, TERT, and TPP1;
(j) PAPR1, PARP2, PARP3, and PARP4;
(k) PARP2, CYP19A1, TEP1, BCL2, HSPA1A, ACE, TP53, and NFKB1;
(l) IGF1, IGF2, PPARG, IL10, APOE, TERT, TNF, HLA-DRA, DDC, CCL4L1, NOS2A, and GH1;
(m) PARP1, IL6, SIRTT1, KRAS, and HSPA1L;
(n) IGF1, IL6, PPARG, IL10, TERT, TNF, TEP1, HSPA1A, SIRT1, TP53, GH1, NOS2A, and PPC;
(o) another list of genes described herein; or
(p) a combination of two or more of (a) through (o).

15. A method of screening compounds useful for modulating lifespan, comprising:
contacting a test compound with a host cell expresses a lifespan-influencing protein encoded by an isolated nucleic acid molecule listed in Data Table 7 or listed as part of Array 2 and
detecting a change in the expression of the nucleotide sequence or a change in activity of encoded protein, wherein such a change indicates the test compound is useful for modulating lifespan.

16. The method of claim 15, which is a high throughput method, comprising:
contacting in parallel a test compound with a collection of host cells each of which expresses a different lifespan-influencing protein encoded by an isolated nucleic acid molecule in listed in Data Table 7 or listed as part of Array 2; and
detecting a change in the expression of at least one of the nucleotide sequences or a change in activity of at least one of the encoding proteins, wherein such a change indicates the test compound(s) are useful for modulating lifespan.

17. A method for identifying an agent with potential to influence mitochondrial damage, comprising:
contacting an cell with an agent; and
detecting the level of a nucleic acid molecule corresponding to:
(1) ACTB, BCL2, BCL2L1, CDKN2A, COX10, COX18, CPT1B, CPT2, DNAJC19, EGF, EGR2, FIST, GAPDH, GRPEL1, HSP90AA1, LRPPRC, MFN1, MFN2, NOS3, OPA1, PARP3, PARP4, PPARGC1A, SIRT2, SIRT4, SLC25A1, SLC25A1, SLC24A2, SLC25A3, SLC25A4, SCL25A5, SLC25A10, SLC25A12, SLC25A13, SLC25A14, SLC25A15, SLC25A16, SLC25A17, SLC25A19, SLC25A2, SLC25A20, SLC25A21, SLC25A22, SLC25A_{—}23, SLC25A24, SLC25A25, SLC25A27, SLC25A3, SLC25A30, SLC25A31, SLC25A37, SLC25A4, SLC25A5, TIMM10, TIMM17A, TIMM17B, TIMM22, TIMM23, TIMM44, TIMM50, TIMM8A, TIMM8B, TIMM9, TOMM20, TOMM22, TOMM34, TOMM40, TOMM40L, TOMM70A, UCP1, UCP2, UCP3 or another gene indicated herein as beneficial for mitochondrial health or maintenance when increased, or the level or activity of a protein encoded thereby, in the presence and absence of the agent, wherein an increase in the level or activity in the presence of the agent as compared to in the absence of the agent indicates that the agent has potential to reverse or inhibit mitochondrial damage; or
(2) AIFM2, AIP, BAK1, BBC3, BID, BNIP3, CLK1, HSPA1A, HSPA1B, HSPA1L, IMMP1L, IMMP2L, MIPEP, PARP1, PARP2, PMAIP1, RPL13A, SOD1, SOD2, SFN, SH3GLB1, UXT or another gene indicated herein as beneficial for mitochondrial health or maintenance when decreased, or the level or activity of a protein encoded thereby, in the presence and absence of the agent, wherein a decrease in the level or activity in the presence of the agent as compared to in the absence of the agent indicates that the agent has potential to reverse or inhibit mitochondrial damage; or
(3) ACTB, BCL2, BCL2L1, CDKN2A, COX10, COX18, CPT1B, CPT2, DNAJC19, EGF, EGR2, FIS1, GAPDH, GRPEL1, HSP90AA1, LRPPRC, MFN1, MFN2, NOS3, OPA1, PARP3, PARP4, PPARGC1A, SIRT2, SIRT4, SLC25A1, SLC25A1, SLC24A2, SLC25A3, SLC25A4, SCL25A5, SLC25A10, SLC25A12, SLC25A13, SLC25A14, SLC25A15, SLC25A16, SLC25A17, SLC25A19, SLC25A2, SLC25A20, SLC25A21, SLC25A22, SLC25A_{—}23, SLC25A24, SLC25A25, SLC25A27, SLC25A3, SLC25A30, SLC25A31, SLC25A37, SLC25A4, SLC25A5, TIMM10, TIMM17A, TIMM17B, TIMM22, TIMM23, TIMM44, TIMM50, TIMM8A, TIMM8B, TIMM9, TOMM20, TOMM22, TOMM34, TOMM40, TOMM40L, TOMM70A, UCP1, UCP2, UCP3 or another gene indicated herein as beneficial for mitochondrial health or maintenance when increased, or the level or activity of a protein encoded thereby, in the presence and absence of the agent, wherein a decrease in the level or activity in the presence of the agent as compared to in the absence of the agent indicates that the agent has potential to increase or accelerate mitochondrial damage; or
(4) AIFM2, AIP, BAK1, BBC3, BID, BNIP3, CLK1, HSPA1A, HSPA1B, HSPA1L, IMMP1L, IMMP2L, MIPEP, PARP1, PARP2, PMAIP1, RPL13A, SOD1, SOD2, SFN, SH3GLB1, UXT or another gene indicated herein as beneficial for mitochondrial health or maintenance when decreased, or the level or activity of a protein encoded thereby, in the presence and absence of the agent, wherein an increase in the level or activity in the presence of the agent as compared to in the absence of the agent indicates that the agent has potential to increase or accelerate mitochondrial damage.

18. The method of claim 17, wherein mitochondrial damage comprises mtDNA depletion or reduced mitochondrial respiratory activity.

19. A method for identifying an agent with potential to influence DNA damage or telomere shortening, comprising:
contacting an cell with an agent; and
detecting the level of a nucleic acid molecule corresponding to:
(1) AK3, APEX1, APEX2, ATF2, ATM, ATR, ATRX, BARD1, BLM, BRIP1, CCNH, CDK7, CDKN2A, CHEK1, CHEK2, CSF2, CTPS, DDB1, DDB2, DHFR, DMC1, ERCC1, ERCC2, ERCC3, ERCC4, ERCC5, ERCC6, ERCC8, EXO1, FANCA, FANCC, FANCF, FANCG, FEN1, GADD45A, GADD45G, GTF2H1, GTF2H2, GTF2H3, GTF2H4, JUN, LIG1, LIG3, LIG4, MAP2K6, MAPKAPK2, MLH1, MLH3, MRE11A, MSH2, MSH3, MSH4, MSH5, MSH6, NBN, NEIL1, NEIL2, NEIL3, NFKB1, NFKBIA, HK1, NUDT1, NUDT2, ODC1, PAPSS1, PAPSS2, PARP1, PARP3, PCNA, PMS1, PMS2, PNKP, POLB, POLD3, POLE, POLI, POLL, PRKDC, RAD1, RAD18, RAD21, RAD23A, RAD50, RAD51C, RAD51L1, RAD51L3, RAD52, RAD54B, RAD54L, RBBP8, SESN1, SLC23A2, TDG, TYMS, UBE2V2, UNG2, WRN, XAB2, XPA, XPC, XRCC1, XRCC2, XRCC3, XRCC4, XRCC5, XRCC6, ZNRD1 or another gene indicated herein as beneficial for DNA or telomere maintenance when increased, or the level or activity of a protein encoded thereby, in the presence and absence of the agent, wherein an increase in the level or activity in the presence of the agent as compared to in the absence of the agent indicates that the agent has potential to reverse or inhibit DNA damage or telomere shortening; or
(2) B2M, BRCA1, BRCA2, BTG2, CIDEA, CIDEB, DDIT3, DKCl, GTSE1, MDM2, PCBP4, PDCD8, PINX1, PPP1R15A, RAD17, RELA, TELO2, TEPlor another gene indicated herein as beneficial for DNA or telomere maintenance when decreased, or the level or activity of a protein encoded thereby, in the presence and absence of the agent, wherein a decrease in the level or activity in the presence of the agent as compared to in the absence of the agent indicates that the agent has potential to reverse or inhibit DNA damage or telomere shortening; or
(3) AK3, APEX1, APEX2, ATF2, ATM, ATR, ATRX, BARD1, BLM, BRIP1, CCNH, CDK7, CDKN2A, CHEK1, CHEK2, CSF2, CTPS, DDB1, DDB2, DHFR, DMC1, ERCC1, ERCC2, ERCC3, ERCC4, ERCC5, ERCC6, ERCC8, EXO1, FANCA, FANCC, FANCF, FANCG, FEN1, GADD45A, GADD45G, GTF2H1, GTF2H2, GTF2H3, GTF2H4, JUN, LIG1, LIG3, LIG4, MAP2K6, MAPKAPK2, MLH1, MLH3, MRE11A, MSH2, MSH3, MSH4, MSH5, MSH6, NBN, NEIL1, NEIL2, NEIL3, NFKB1, NFKBIA, HK1, NUDT1, NUDT2, ODC1, PAPSS1, PAPSS2, PARP1, PARP3, PCNA, PMS1, PMS2, PNKP, POLB, POLD3, POLE, POLI, POLL, PRKDC, RAD1, RAD18, RAD21, RAD23A, RAD50, RAD51C, RAD51L1, RAD51L3, RAD52, RAD54B, RAD54L, RBBP8, SESN1, SLC23A2, TDG, TYMS, UBE2V2, UNG2, WRN, XAB2, XPA, XPC, XRCC1, XRCC2, XRCC3, XRCC4, XRCC5, XRCC6, ZNRD1 or another gene indicated herein as beneficial for DNA or telomere maintenance when increased, or the level or activity of a protein encoded thereby, in the presence and absence of the agent, wherein a decrease in the level or activity in the presence of the agent as compared to in the absence of the agent indicates that the agent has potential to accelerate or cause or enhance DNA damage or telomere shortening; or
(4) B2M, BRCA1, BRCA2, BTG2, CIDEA, CIDEB, DDIT3, DKCl, GTSE1, MDM2, PCBP4, PDCD8, PINX1, PPP1R15A, RAD17, RELA, TELO2, TEPlor another gene indicated herein as beneficial for DNA or telomere maintenance when decreased, or the level or activity of a protein encoded thereby, in the presence and absence of the agent, wherein an increase in the level or activity in the presence of the agent as compared to in the absence of the agent indicates that the agent has potential to accelerate or cause or enhance DNA damage or telomere shortening.
